(19) United States
(12) Patent Application Publication
(10) Pub. No.: US 2025/0011368 A1
(43) Pub. Date: Jan. 9, 2025

(54) DLL3 TARGETING PEPTIDES AND CONSTRUCTS THEREOF

(71) Applicant: MARIANA ONCOLOGY INC., Cambridge, MA (US)

(72) Inventors: Chester A. METCALF, III, Needham, MA (US); Chunhui Huang, Arlington, MA (US); Murray Wan, Medford, MA (US); Thomas C. Bruton, Newmarket, NH (US); Zhong Ma, Bedford, MA (US); Lihua Wu, Belmont, MA (US); Roberto Costante, Pomezia (RM) (IT); Danila Branca, Pomezia (RM) (IT); Alonso Ricardo, Winchester, MA (US)

(21) Appl. No.: 18/743,021

(22) Filed: Jun. 13, 2024

Related U.S. Application Data
(60) Provisional application No. 63/557,140, filed on Feb. 23, 2024.
(60) Provisional application No. 63/508,202, filed on Jun. 14, 2023.

(51) Int. Cl. C07K 7/64; A61K 38/00

(52) U.S. Cl. CPC C07K 7/64; A61K 38/00

(57) ABSTRACT

The present disclosure relates to targeting moieties such as peptides and antibodies that can bind to DLL3. The disclosure also provides targeting constructs, which may include a targeting moiety attached, via an optional linker, to a chelating agent for association of a cargo. Methods of making the constructs and formulations thereof are also provided. Methods of using the constructs and/or formulations thereof to treat subjects, for example, to treat or prevent cancer, are also described.

### RELATED APPLICATIONS

This application is related to U.S. Provisional Application No. 63/508,202 filed Jun. 14, 2023, and U.S. Provisional Application No. 63/557,140 filed Feb. 23, 2024, the entire content of which is incorporated by reference in its entirety. The contents of each application are hereby incorporated by reference in their entireties.

### REFERENCE TO SEQUENCE LISTING SUBMITTED ELECTRONICALLY

This application contains a computer readable Sequence Listing, which has been submitted in XML file format with this application, the entire content of which is incorporated by reference herein in its entirety. The Sequence Listing XML file submitted with this application is entitled “755424 CTT-010PC.xml”, was created on Jul. 19, 2024, and is 870,044 bytes in size.

### BACKGROUND

Radiation therapy or radiotherapy is a cancer treatment that uses high doses of radiation to kill cancer cells and shrink tumors. In recent years, targeted radionuclide therapies for cancer utilizing radiolabeled peptides have been developed as an alternative to external radiation therapies. These peptides typically bind to receptors overexpressed by cancer cells. Despite these advancements, there remains a need for new targeted radionuclide therapies.

Delta-like ligand 3 (DLL3), a member of the Notch signaling system, is a potential target for radionuclide therapies. This evolutionarily conserved system regulates cell fate via cell-cell interactions. During embryonic development, DLL3 is highly expressed and transported to the cell membrane. Once development is complete, DLL3 expression is downregulated and confined to the inside of the cell, typically the Golgi apparatus. DLL3 expression, however, has been found to be highly expressed and localized to the cell membrane in many forms of cancer (Xiu et al., Onco. Targets Ther. (2020), 13:3881-3901).

In addition to being a biomarker, DLL3 plays a role in the regulation of cancer behavior. A study of small cell lung cancer (SCLC) showed that upregulation of DLL3 expression reduced the tumor's sensitivity to chemotherapy. Additionally, by blocking DLL3, the proliferation and migration of SCLC cells was inhibited and the epithelial to mesenchymal transition (EMT) was reversed (Huang et al., Biochem. Biophys. Res. Commun. (2019) 514(3):853-860). The oncogenic behavior of DLL3 has also been documented in pancreatic cancer, melanoma, and gastric cancer (Mullendore, et al., Clin. Cancer. Res. (2009) 15(7):2291-301; Ding, et al., Life Sci. (2019) 226:149-155; Hu et al., Nan Fang Yi Ke Da Xue Xue Bao. (2018) 38(1):14-19).

Taken together, these findings suggest that DLL3 plays a critical role in the regulation of oncogenic pathways and is specifically upregulated in cancer cells; development of treatments targeting DLL3 are therefore useful in the clinical treatment of cancer.

### SUMMARY

The present disclosure relates to targeting moieties such as peptides, proteins and antibodies that can bind to delta like canonical Notch ligand 3 (DLL3). The disclosure also provides targeting constructs, which may include a targeting moiety attached, via an optional linker, to a chelating agent for association of a cargo. The chelator can be associated with a payload such as, e.g., a radionuclide or cytotoxic agent. In a particular aspect, the targeting construct comprises a targeting moiety that is a cyclic peptide that targets DLL3, which is attached, via an optional linker, to a chelating agent for association of a radioisotope or radionuclide.

Accordingly, provided herein are cyclic peptides that target DLL3. As such, these peptides are useful in the treatment of a variety of indications, including cancer.

In an aspect, provided herein is a cyclic peptide comprising the amino acid sequence of Formula A:

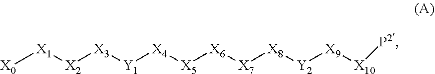

or a pharmaceutically acceptable salt thereof, wherein the cyclic peptide is cyclized via a linker between Y₁ and Y₂, wherein the remaining variables are defined herein, wherein the chelator is optionally linked to the cyclic peptide via a linking group; and wherein the cyclic peptide binds to DLL3. The chelator can be labeled with a radionuclide.

In another aspect, provided herein is a cyclic peptide of Formula I:

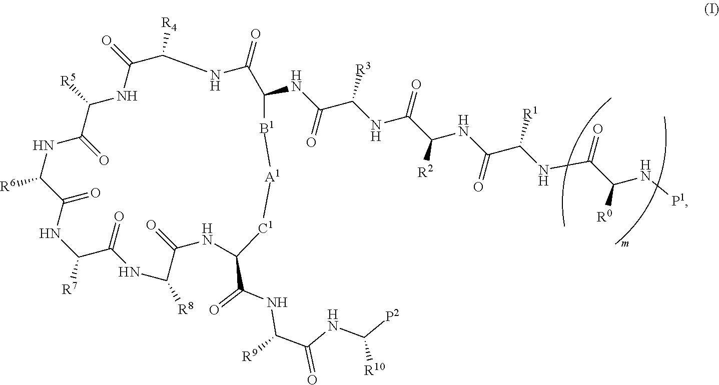

or a pharmaceutically acceptable salt thereof, where the variables are defined herein.

In an embodiment, the cyclic peptide of Formula I is attached, via an optional linker, to a chelating agent for association of a radionuclide.

In another embodiment, the cyclic peptide of Formula I is selected from a cyclic peptide in Table A. In yet another embodiment, the cyclic peptide of Formula I is selected from a cyclic peptide in Table B.

In another embodiment, the cyclic peptide of Formula I is selected from a cyclic peptide in Table A, or a pharmaceutically acceptable salt and/or solvate thereof. In yet another embodiment, the cyclic peptide of Formula I is selected from a cyclic peptide in Table B, or a pharmaceutically acceptable salt and/or solvate thereof.

In yet another aspect, provided herein is a cyclic peptide of Formula B:

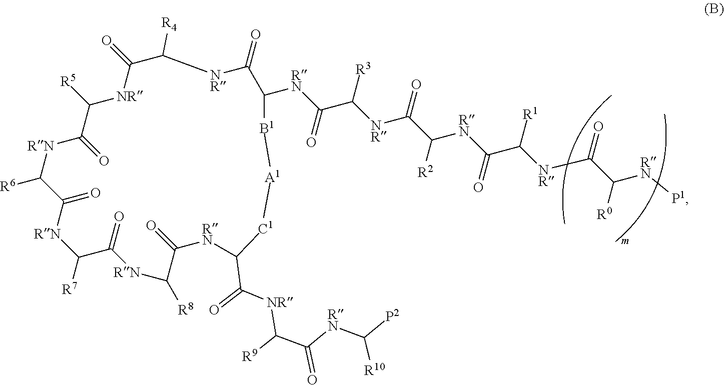

or a pharmaceutically acceptable salt thereof, where the variables are defined herein.

In an embodiment, the cyclic peptide of Formula B is attached, via an optional linker, to a chelating agent for association of a radioisotope.

In another embodiment, the cyclic peptide of Formula B is selected from a cyclic peptide in Table A. In yet another embodiment, the cyclic peptide of Formula B is selected from a cyclic peptide in Table B.

In another embodiment, the cyclic peptide of Formula B is selected from a cyclic peptide in Table A, or a pharmaceutically acceptable salt and/or solvate thereof. In yet another embodiment, the cyclic peptide of Formula B is selected from a cyclic peptide in Table B, or a pharmaceutically acceptable salt and/or solvate thereof.

In still another embodiment, the chelating agent is selected from a chelating agent in Table C. In some embodiments, the chelating agent is labeled with a radionuclide. In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y ⁸⁹Zr, ⁵¹Cr, ⁵²Mn, ⁵¹Mn, ¹⁷⁷Lu ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th.

In an embodiment, the radioisotope is selected from a radioisotope in Table 3.

In another aspect, provided herein is a pharmaceutical composition comprising a cyclic peptide described herein, or a pharmaceutically acceptable salt thereof, and a pharmaceutically acceptable carrier.

In yet another aspect, provided herein is a method of targeting DLL3 in a subject in need thereof comprising administering to the subject a therapeutically effective amount of a compound described herein.

In still another aspect, provided herein is a method of treating cancer in a subject in need thereof comprising administering to the subject a therapeutically effective amount of a compound described herein. The cancer may include at least one cell comprising DLL3. The cancer may be urothelial cancer, melanoma or squamous cell carcinoma. Additionally, the cancer can be a neuroendocrine neoplasm, melanoma, or primary brain cancer. In some embodiments, the neuroendocrine neoplasm is selected from small cell lung cancer (SCLC), medullary thyroid carcinoma (MTC), large cell neuroendocrine cancer (LCNEC), gastroenteropancreatic neuroendocrine carcinoma (GEP NEC), neuroendocrine prostate cancer (NEPC), small cell prostate cancer (SCPC), Merkel cell carcinoma (MCC), neuroendocrine cervical carcinoma, Grade 3 neuroendocrine tumors (NETs), and extrapulmonary neuroendocrine carcinoma (NEC) of the cervix. In some embodiments, the cancer can be a solid tumor having DLL3 positivity as measured by immunohistochemistry (IHC) (e.g., ≥1% DLL3 positive cells).

In an aspect, provided herein is a peptide having binding specificity for DLL3, wherein the peptide binds to one or more amino acids of A81, L83, G106, A85, and R61 of a DLL3 amino acid sequence of SEQ ID NO: 1. In an embodiment, the peptide binds to amino acids A81, L83, G106, A85, and R61 of a DLL3 amino acid sequence of SEQ ID NO: 1. In another embodiment, the peptide binds to main chain atoms of amino acids A81, L83, G106, and A85 of a DLL3 amino acid sequence of SEQ ID NO: 1. In yet another embodiment, the peptide binds to side chain atoms of amino acid R61 of a DLL3 amino acid sequence of SEQ ID NO: 1. In still another embodiment, the peptide binds to main chain atoms of amino acids A81, L83, G106, and A85 of a DLL3 amino acid sequence of SEQ ID NO: 1, and binds to side chain atoms of amino acid R61 of a DLL3 amino acid sequence of SEQ ID NO: 1. In an embodiment, the peptide comprises an amino acid sequence of WTACANAKDCWP, or a derivative thereof comprising one or more unnatural amino acids. In another embodiment, amino acids W1, A3, A7, and W11 bind to DLL3. In yet another embodiment, the peptide is cyclic. In some embodiments, the present disclosure provides a construct comprising a targeting moiety attached, via an optional linker, to at least one chelating agent for association of a cargo, or a pharmaceutically acceptable salt thereof, wherein the targeting moiety binds to a cell antigen, wherein the cell antigen comprises DLL3. The cargo can be a payload such as, e.g., a radionuclide or cytotoxic agent.

In some embodiments, the present disclosure provides a construct including a targeting moiety attached, via an optional linker, to at least one chelating agent for association of a cargo, or a pharmaceutically acceptable salt thereof, wherein the targeting moiety binds to DLL3. The chelating agent may include a polyaminocarboxylate agent. The chelating agent may include ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), DOTAGA, or a derivative thereof. The chelating agent can comprise EDTA, DTPA, DOTA, DOTAGA, or a derivative thereof. The chelating agent may include a macrocyclic agent. The chelating agent may include 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA), or a derivative thereof. The chelating agent can also include deferoxamine (DFO), 5,11,16,22-tetraazahexacosanediamide (DFO*) or N,N′-1,4-Butanediylbis[N-[3-[[(1,6-dihydro-1-hydroxy-6-oxo-2-pyridinyl)carbonyl]amino]propyl]-1,6-dihydro-1-hydroxy-6-oxo-2-pyridinecarboxamide] (HOPO), or a derivative thereof.

The cargo may include a radioactive agent. The radioactive agent may include a radioisotope. Accordingly, in some embodiments, the constructs or compounds disclosed herein optionally comprise a radioisotope. In some embodiments, the constructs or compounds disclosed herein comprise a radioisotope. The radioisotope can be a radionuclide. The radioisotope may be any of those listed in Table 3. In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th. The optional linker may include a cleavable linker. The optional linker may include a non-cleavable linker. The optional linker may comprise at least one amino acid. In some embodiments, the present disclosure provides a pharmaceutical composition including a construct and a pharmaceutically acceptable excipient.

In some embodiments, the present disclosure provides a method of delivering a cargo to a cell that includes contacting the cell or a subject comprising the cell with a construct or the pharmaceutical composition thereof. In an embodiment, the cargo can be a radioactive agent, such as a radionuclide and/or radioisotope. In other embodiments, the cargo is a cytotoxic agent.

In some embodiments, the present disclosure provides a method of treating a disease or disorder in a subject comprising administering a construct or the pharmaceutical composition thereof. In some embodiments, the present disclosure provides a method of treating a subject that includes administering a construct or the pharmaceutical composition thereof. In some embodiments, the disease or disorder is cancer (i.e., the subject has cancer). The cancer may include at least one cell comprising DLL3. In an embodiment, the cancer expresses DLL3. The cancer may be urothelial cancer, melanoma or squamous cell carcinoma. Additionally, the cancer can be a neuroendocrine neoplasm, melanoma, or primary brain cancer. In some embodiments, the neuroendocrine neoplasm is selected from small cell lung cancer (SCLC), medullary thyroid carcinoma (MTC), large cell neuroendocrine cancer (LCNEC), gastroenteropancreatic neuroendocrine carcinoma (GEP NEC), neuroendocrine prostate cancer (NEPC), small cell prostate cancer (SCPC), Merkel cell carcinoma (MCC), neuroendocrine cervical carcinoma, Grade 3 neuroendocrine tumors (NETs), and extrapulmonary neuroendocrine carcinoma (NEC) of the cervix. In some embodiments, the cancer can be a solid tumor having DLL3 positivity by immunohistochemistry (IHC) (e.g., ≥1% DLL3 positive cells).

### DETAILED DESCRIPTION

DLL3 binds members of the highly conserved notch receptor family to regulate embryonic development. In contrast to the canonical notch ligands, DLL3 suppresses Notch signaling through interactions with the Golgi. Reflecting this function, DLL3 is normally confined to the cytoplasm (Geffers, I., et al. J. Cell Biol. (2007) 178(3), 465-76; Zhou, B., et al. Signal Transduct. Target Ther. (2022) 7(1), 95). DLL3 is detected in the cytoplasm of healthy fetal tissues and its absence leads to severe vertebral defects, in the form of autosomal recessive spondylocostal dysostosis (Serth, K., et al. PLoS One, (2015) 10(4), e0123776; Dunwoodie, S. L., et al. Development, (2002) 129(7), 1795-806). Low levels of DLL3 are detectable as an RNA transcript in adult brain, pituitary and testis (Sharma, S. K., et al. Cancer Res. (2017) 77(14), 3931-41). DLL3 mRNA is also detectable in the cytoplasm of adult brain, pituitary, basophils, and pancreas (Giffin, M. J., et al. Clin. Cancer Res, (2021) 27(5), 1526-37). Significant overexpression of DLL3, however, results in its aberrant localization to the cell surface (Zhou, B., et al. Signal Transduct. Target Ther. (2022) 7(1), 95; Geffers, I., et al. (2007)). Upregulated DLL3 is also observed aberrantly localized to the cell surface in cancer (Giffin, M. J., et al. (2021); Saunders, L. R., et al. Sci. Transl. Med. (2015) 7(302), 302ra136; Sharma, S. K., et al. (2017)).

Abnormal DLL3 expression including cell surface expression is observed in a variety of human tumors (Saunders, L. R., et al. (2015)). Neuroendocrine neoplasms (NENs), including well differentiated neuroendocrine tumors (NETs) and poorly differentiated neuroendocrine carcinomas (NECs), frequently express DLL3 on the cell surface and share common histologic and transcriptomic markers of neuroendocrine lineage and transformation (Puca, L., et al. Sci. Transl. Med. (2019) 11(484); Yao, J., et al. Oncologist (2022) 27(11), 940-51). The pathophysiological role of DLL3 mis-localization on tumor cell function is poorly understood, although gain and loss of function experiments suggest that DLL3 may impact cell proliferation, migration, and tumor growth in vitro and in vivo (Furuta, M., et al. Cancer Sci. (2019) 110(5), 1599-608; Huang, J., et al. (2019). DLL3 is detectable on tumor cell surface by immunohistochemistry (IHC), flow cytometry, and is accessible in vivo to exogenous DLL3-targeted antibodies (Dylla, S. J. Mol. Cell Oncol. (2016) 3(2), e1101515; Saunders, L. R., et al. (2015)).

DLL3 has been quantified in the low single-digit thousands of copies per cell in representative small cell lung cancer (SCLC) and neuroendocrine prostate cancer (NEPC) cell lines (Giffin, M. J., et al. (2021); Zhang, Y., et al. Clin. Cancer Res. (2023) 29(5), 971-85). Despite its low copy number levels on a per cell basis, the feasibility of targeting DLL3 has nonetheless been demonstrated preclinically in SCLC as well as in neuroendocrine cancer xenograft models, using DLL3 targeted 89Zr/177Lu radio-conjugates, DLL3 targeted bispecifics, and DLL3 targeted antibody drug conjugates (Chou, J., et al. Cancer Res. (2023) 83(2), 301-15; Giffin, M. J., et al. (2021); Korsen, J. A., et al. Proc. Natl. Acad. Sci. USA, (2022) 119(27), e2203820119; Saunders, L. R., et al. (2015)). Furthermore, despite the low abundance of the tumor-associated antigen, visualization of DLL3-expressing tumors through 89Zr immunoPET imaging was demonstrated in several mouse models of SCLC (Sharma, S. K., et al. (2017)).

Its clinical validation as a target has been confirmed with DLL3 targeting therapies including a DLL3 targeted antibody drug conjugate RovaT (Morgensztern, D., et al. Clin Cancer Res, (2019) 25(23), 6958-66; Rudin, C. M., et al. Nat Rev Dis Primers (2021) 7(1), 3), which was limited in its potential due to payload-related toxicity. More recently, a DLL3 targeted bispecific tarlatamab (AMG757) has yielded durable responses up to 12-months in a quarter of treated patients (Paz-Ares, L., et al. J. Clin. Oncol. (2023) 41(16), 2893-903). Given its well-studied nature and its selective expression at the surface of cancer cells in both primary and metastatic solid tumors, DLL3 is a compelling target for novel therapies in NENs and other solid tumors expressing DLL3.

Provided herein are compounds that target DLL3. In particular, provided herein are targeting constructs (also referred to herein as “compounds”) comprising a targeting moiety that is a cyclic peptide that targets DLL3, which is attached, via an optional linker, to a chelating agent for association of a radioisotope. As such, these compounds, as well as pharmaceutical compositions that comprise these compounds, are useful in the treatment of a variety of indications, including cancer.

### 1. Compounds and Compositions

In some embodiments, the present disclosure relates to targeting moieties such as peptides, proteins and antibodies that can bind to targets. In some embodiments, the present disclosure provides constructs capable of localizing to and/or associating with targets. Such constructs that include any combination of a targeting moiety and a cargo are referred to herein as “targeting constructs.” As used herein, the term “targeting moiety” refers to a component of a targeting construct or combination of components involved in targeting construct localization to or association with a target. Cargo components of targeting constructs may include any one of a variety of compounds, including, but not limited to, chemical compounds, biomolecules, metals, polymeric molecules, therapeutic agents, cytotoxic agents, and radioactive agents. The chelator can be associated with a payload such as, e.g., a radionuclide or cytotoxic agent.

In particular, provided herein are targeting constructs (also referred to herein as “compounds”) comprising a targeting moiety that is a cyclic peptide that targets DLL3, which is attached, via an optional linker, to a chelating agent for association of a radioisotope.

### Targets

Targeting constructs may be directed to a variety of targets. In a particular embodiment, the targeting construct comprises a peptide directed to DLL3 and also comprises a radioisotope. In some embodiments, targeting constructs may target cells. Such targeting constructs may include targeting moieties that may target cell antigens, including those associated with target cell surfaces. In this case, the cell antigen is the target of the targeting moieties and the targeting constructs. An “antigen,” as referred to herein, is any entity that induces an immune response in an organism or may simply refer to an antibody binding partner. Immune responses are reactions of cells, tissues and/or organs of an organism to a foreign entity. Immune responses typically lead to the production of one or more antibodies against a foreign entity by an organism. As used herein, the term “target antigen” refers to an entity, protein, or epitope to which an antibody binds or for which an antibody is desired, designed, or developed to have affinity for. Such target antigens may include cancer cell antigens, for example, those expressed on cancer cell surfaces.

In some embodiments, target antigens of the present disclosure include DLL3 or portions thereof. DLL3 antigens may include DLL3 extracellular domains. DLL3 antigens may include fusion proteins of DLL3 or other entities comprising DLL3 portions.

### DLL3

Delta Like Canonical Notch Ligand 3 (also known as Delta-like protein 3, Drosophila, or DLL3) is a member of the delta protein ligand family. It is encoded by the DLL3 gene. DLL3 inhibits primary neurogenesis and is involved in diverting neurons along a specific differentiation pathway. It also plays a role in the formation of somite boundaries during segmentation of the paraxial mesoderm. Mutations in the DLL3 gene cause the autosomal recessive genetic disorder Jarcho-Levin syndrome. Expression of the DLL3 gene occurs in neuroendocrine tumors. DLL3 can be a potential target for treating tumors such as lung cancer.

In some embodiments, targeting constructs include targeting moieties specific for one or more DLL3 domains. In some embodiments, targeting constructs include targeting moieties specific for one or more DLL3 target amino acid sequence listed in Table 1 or a fragment or variant thereof.

TABLE 1
DLL3 protein target sequences

| Sequence | | SEQ |
|---|---|---|
| descrip- | | ID |
| tion | Sequence | NO |
| DLL3 | MVSPRMSGLLSQTVILALIFLPQTRPAGVFELQIHSFGPGPGPGAPRS | 1 |
| | PCSARLPCRLFFRVCLKPGLSEEAAESPCALGAALSARGPVYTEQPG | |
| | APAPDLPLPDGLLQVPFRDAWPGTFSFIIETWREELGDQIGGPAWSLL | |
| | ARVAGRRRLAAGGPWARDIQRAGAWELRFSYRARCEPPAVGTACTR | |
| | LCRPRSAPSRCGPGLRPCAPLEDECEAPLVCRAGCSPEHGFCEQPG | |
| | ECRCLEGWTGPLCTVPVSTSSCLSPRGPSSATTGCLVPGPGPCDGN | |
| | PCANGGSCSETPRSFECTCPRGFYGLRCEVSGVTCADGPCFNGGLC | |
| | VGGADPDSAYICHCPPGFQGSNCEKRVDRCSLQPCRNGGLCLDLGH | |
| | ALRCRCRAGFAGPRCEHDLDDCAGRACANGGTCVEGGGAHRCSCA | |
| | LGFGGRDCRERADPCAARPCAHGGRCYAHFSGLVCACAPGYMGAR | |
| | CEFPVHPDGASALPAAPPGLRPGDPQRYLLPPALGLLVAAGVAGAAL | |
| | LLVHVRRRGHSQDAGSRLLAGTPEPSVHALPDALNNLRTQEGSGDG | |
| | PSSSVDWNRPEDVDPQGIYVISAPSIYAREVATPLFPPLHTGRAGQRQ | |
| | HLLFPYPSSILSVK | |
| DLL3 | AGVFELQIHSFGPGPGPGAPRSPCSARLPCRLFFRVCLKPGLSEEAA | 2 |
| (amino | ESPCALGAALSARGPVYTEQPGAPAPDLPLPDGLLQVPFRDAWPGTF | |
| acids 27- | SFIIETWREELGDQIGGPAWSLLARVAGRRRLAAGGPWARDIQRAGA | |
| 492) | WELRFSYRARCEPPAVGTACTRLCRPRSAPSRCGPGLRPCAPLEDE | |
| | CEAPLVCRAGCSPEHGFCEQPGECRCLEGWTGPLCTVPVSTSSCLS | |
| | PRGPSSATTGCLVPGPGPCDGNPCANGGSCSETPRSFECTCPRGFY | |
| | GLRCEVSGVTCADGPCFNGGLCVGGADPDSAYICHCPPGFQGSNCE | |
| | KRVDRCSLQPCRNGGLCLDLGHALRCRCRAGFAGPRCEHDLDDCAG | |
| | RACANGGTCVEGGGAHRCSCALGFGGRDCRERADPCAARPCAHGG | |
| | RCYAHFSGLVCACAPGYMGARCEFPVHPDGASALPAAPPGLRPGDP | |
| | QRYL | |

### Targeting Moieties

In some embodiments, targeting moieties localize targeting constructs to targets by binding such targets or associated components. Targeting moieties may bind to cells or biomolecules or other structures associated with cells. For example, in some embodiments, targeting moieties bind to cell antigens. Such cell antigens may be specifically expressed by, expressed on, or otherwise associated with specific cell types. Specific cell types may be characterized by one or more of cell size, age, shape, location, tissue of origin, organ of origin, function, activity, genotype, phenotype, or association with disfunction or disease. Targeting moieties may bind to cancer cell antigens. In some embodiments, targeting moieties bind to DLL3. In some embodiments, targeting moieties bind to human DLL3.

Targeting moieties may include or consist of proteins, peptides, antibodies, nucleic acids, nucleic acid analogs, aptamers, lipids, carbohydrates, glycoproteins, or small molecules. In some embodiments, the targeting moieties include or consist of polypeptides or peptides, antibodies or fragments or variants thereof. In a particular embodiment, the targeting moiety is a cyclic peptide.

In some embodiments, targeting moieties of the disclosure, such as peptides, and antibodies have an affinity for human DLL3. In some embodiments, targeting moieties of the disclosure have an affinity for human DLL3 within identified ranges as measured in conventional assays. “Affinity” or “binding affinity” means the strength of the sum total of noncovalent interactions between a single binding site of a molecule (e.g., an antibody or peptide binding compound) and its binding partner (e.g., an antigen). Unless indicated otherwise, as used herein, “binding affinity” refers to intrinsic binding affinity which reflects all interaction between members of a binding pair (e.g., antibody or peptide binding compound and antigen). The affinity of a molecule X for its partner Y can generally be represented by the dissociation constant (K_{D}). The equilibrium dissociation constant (K_{D}) is calculated as the ratio k_{off}/kₒₙ.

Low-affinity targeting moieties generally bind antigen slowly and tend to dissociate readily, whereas high-affinity targeting moieties generally bind antigen faster and tend to remain bound longer. A variety of methods of measuring binding affinity are known in the art, any of which can be used for purposes of the present disclosure.

In some embodiments, the targeting moieties disclosed herein, may bind to a target protein with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM or at least 1,000 nM. In some embodiments, the target protein is DLL3.

In some embodiments, the targeting moieties disclosed herein, can bind to a target protein with an equilibrium dissociation constant (K_{D}) of about 1 nM or less, about 1 nM to about 10 nM, about 10 nM to about 100 nM, or about 100 nM to about 300 nM. In some embodiments, the target protein is DLL3.

In some embodiments, the K_{D} is determined by Surface Plasmon Resonance (SPR). An exemplary SPR protocol is provided in Example 3.

### Polypeptide and Peptides

In some embodiments, targeting moieties of the present disclosure are polypeptides. According to the present disclosure, any amino acid-based molecule (natural or unnatural) may be termed a “polypeptide” and this term embraces “peptides,” “peptidomimetics,” and “proteins.” “Peptides” are traditionally considered to range in size from about 4 to about 50 amino acids. Peptides larger than about 50 amino acids are generally termed “proteins.”

Peptides of the present disclosure may be linear or cyclic. In particular, provided herein are cyclic peptides that target DLL3. Cyclic peptides include any peptides that have as part of their structure one or more cyclic features such as a loop and/or an internal linkage. In some embodiments, cyclic peptides are formed when a molecule acts as a bridging moiety to link two or more regions of the peptide.

As used herein, the term “bridging moiety” refers to one or more components of a bridge formed between two adjacent or non-adjacent amino acids, unnatural amino acids or non-amino acids in a peptide. Bridging moieties may be of any size or composition. In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof. Bridging moieties may include one or more of an amide bond (lactam), disulfide bond, thioether bond, aromatic ring, triazole ring, and hydrocarbon chain. In some embodiments, bridging moieties include an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue.

In some embodiments, the present disclosure provides peptides that bind to human DLL3. Antibodies of the present disclosure may bind human DLL3 extracellular domains. Antibodies of the present disclosure may bind DLL3 associated with cells (e.g., cell surfaces). Such cells may include cancer cells, such as but not limited to lung cancer cells, breast cancer cells, bladder cancer cells, colon cancer cells, urothelial cancer cells, melanoma cells, or squamous cell carcinoma cells.

### DLL3 Epitope

The peptides of the disclosure that bind to human DLL3 bind to specific amino acids in DLL3, i.e., a DLL3 epitope. As used herein, the term “epitope” refers to the specific portion(s) of a target (e.g., DLL3) which interact (e.g., bind) with a binding entity (e.g., the peptides of the disclosure).

In some embodiments, the peptides having binding specificity for DLL3, wherein the peptide binds to one or more amino acids of A81, L83, G106, A85, and R61 of a DLL3 amino acid sequence of SEQ ID NO: 1.

In some embodiments, the peptides bind to amino acids A81, L83, G106, A85, and R61 of a DLL3 amino acid sequence of SEQ ID NO: 1.

In some embodiments, the peptides bind to main chain atoms of amino acids A81, L83, G106, and A85 of a DLL3 amino acid sequence of SEQ ID NO: 1. As used herein, the term “main chain atoms” refers to the atoms in the peptide backbone, which is composed of a central carbon atom (the alpha carbon) bonded to a hydrogen atom, an amino group (NH₂), and a carboxyl group (COOH).

In some embodiments, the peptides bind to side chain atoms of amino acid R61 of a DLL3 amino acid sequence of SEQ ID NO: 1. As used herein, the term “side chain atoms” refers to the atoms in the peptide side chain, also commonly referred to as the R group,

In some embodiments, the peptides bind to main chain atoms of amino acids A81, L83, G106, and A85 of a DLL3 amino acid sequence of SEQ ID NO: 1, and binding to side chain atoms of amino acid R61 of a DLL3 amino acid sequence of SEQ ID NO: 1.

In another embodiment, the peptides are capable of binding DLL3 with an EC₅₀ value of about 1×10⁻⁸ M to about 1×10⁻¹² M. In another embodiment, the peptides are capable of binding DLL3 with an EC₅₀ value of about 1×10⁻⁸ M to about 1×10⁻¹⁰ M.

In some embodiments, the EC₅₀ value is determined in an enzyme-linked immunosorbent assay (ELISA). In some embodiments, a DLL3 protein concentration of about 1 μg/mL to about 5 μg/mL is used in the ELISA.

In some embodiments, the peptides comprise an amino acid sequence of WTACANAKDCWP, or a derivative thereof comprising one or more unnatural amino acids. In another embodiment, amino acids W1, A3, A7, and W11 bind to DLL3. In yet another embodiment, the peptide is cyclic. In still another embodiment, the peptide is any of the formulae or species described herein.

### Cyclic Peptides

In some embodiments, peptides of the present disclosure can comprise cyclic peptides having one or more bridging moieties (e.g., cyclic structure, staple, bridge, etc.). Peptide stapling/bridging is a macrocyclization approach in which peptides are covalently modified through the formation of a chemical linkage (e.g., staple, bridge moiety, etc.) between the side chains of two amino acids. More specifically, peptides are rendered macrocyclic by formation of covalent bonds between atoms present within the linear peptide and atoms of a bridging moiety. Stapling/bridging can be used to constrain peptides into preferred bioactive conformations (reducing conformational flexibility and degrees of rotational freedom), thereby improving affinity for specific receptor targets and improving overall pharmacokinetics. The residues being linked are generally located on the same face of the peptide helix and separated by one, two, or three helical turns (e.g., a first amino acid at position (z) is linked to a second amino acid at position z+4, z+7, or z+11). In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof.

Accordingly, in an aspect, provided herein is a cyclic peptide comprising the amino acid sequence of Formula A:

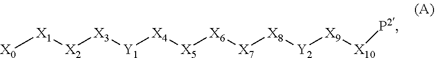

or a pharmaceutically acceptable salt thereof,
wherein:
X₀ is any natural or unnatural amino acid or X₀ is absent;
X₁ is selected from Trp, 7Aza-Trp, 1Me-Trp, 5OH-Trp, 5OMe-Trp, 7OMe-Trp, 7Me-Trp, 5F-Trp, 7Cl-Trp, alpha-Me-Trp, and NMe-Trp;
X₂ and X₃ are each independently any natural or unnatural amino acids;
Y₁ is Cys;
X₄, X₅, X₆, X₇, and X₈ are each independently any natural or unnatural amino acids;
Y₂ is Cys;
X₉ is selected from Trp, 7Aza-Trp, 1Me-Trp, 5OH-Trp, 5OMe-Trp, 7OMe-Trp, 7Me-Trp, 5F-Trp, 7Cl-Trp, alpha-Me-Trp, and NMe-Trp;
X₁₀ is selected from Pro, alpha-Me-Pro, trans4Fluoro-Pro, cis4Fluoro-Pro, trans40H-Pro, cis4OH-Pro, 5,5-diMe-Pro, trans4NH2-Pro, and cis4NH2-Pro;
P^{2′} is selected from -L²-Chelator or

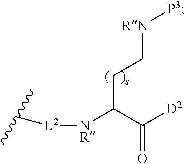

D² is OH or NH₂;
L² is absent or selected from:

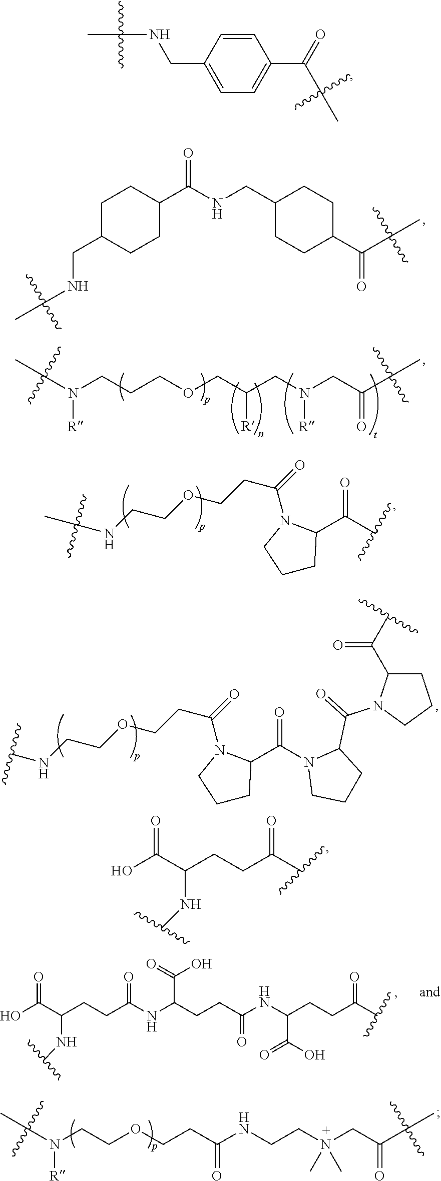

wherein the amino group of L² connects to the carbonyl group of P² or Chelator to form an amide bond;
P³ is selected from -L³-Chelator,

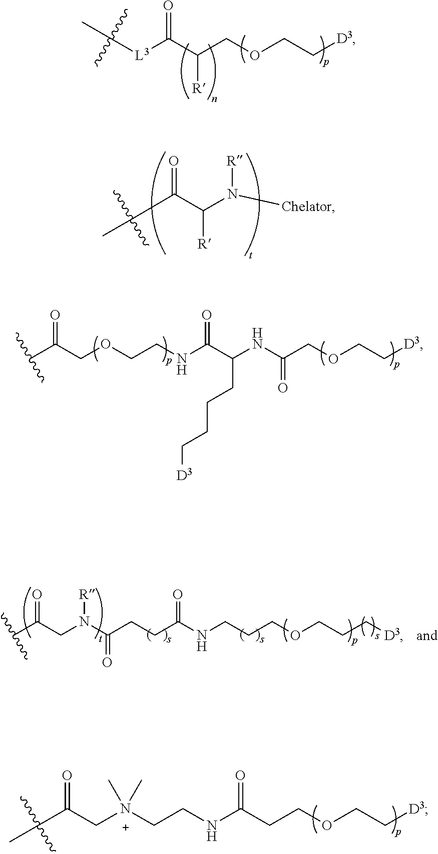

L³ is absent or independently selected from

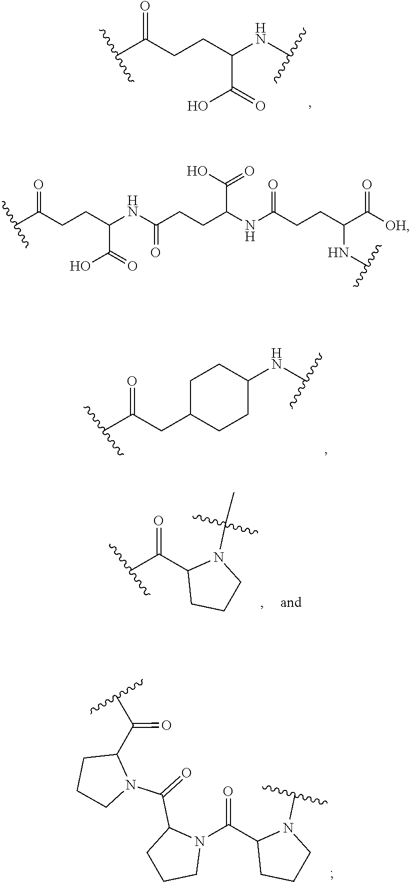 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the carbonyl group of L³ connects to an amine group of P² to form an amide bond;
D³ is independently —NR″-Chelator or

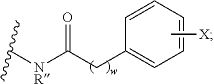

each R′ is independently selected from H, C(O)OH, (CH₂)OH, and NHAc;
each R″ is independently selected from H and CH₃;
X is H or halogen;
each n is independently an integer from 0 to 16;
each p is independently an integer from 0 to 24;
each s is independently an integer from 0 to 16;
each t is independently 1, 2, 3, 4, 5, or 6; and
w is selected from 1, 2, or 3;
wherein the cyclic peptide is cyclized via a linker between Y₁ and Y₂; and
wherein the cyclic peptide binds to DLL3.

In an embodiment, X₂ is Thr and X₈ is Asp.

In another embodiment, the cyclic peptide of Formula A is a cyclic peptide comprising the amino acid sequence of Formula Ai:

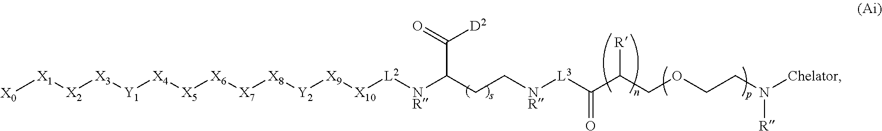

or a pharmaceutically acceptable salt thereof.

In yet another embodiment, the cyclic peptide of Formula A is a cyclic peptide comprising the amino acid sequence of Formula Aii:

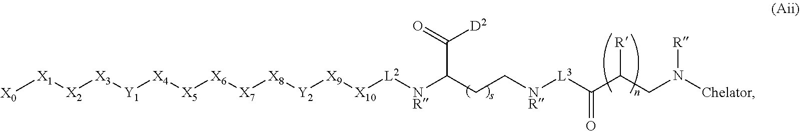

or a pharmaceutically acceptable salt thereof.

In yet another embodiment, the cyclic peptide of Formula A is a cyclic peptide comprising the amino acid sequence of Formula Aiii:

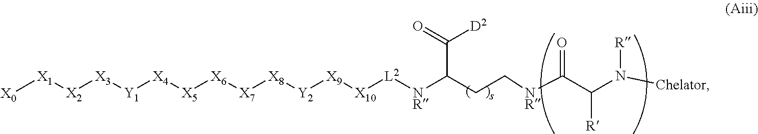

or a pharmaceutically acceptable salt thereof.

In another embodiment, the N-terminus of the peptide is capped. In still another embodiment, the N-terminus of the peptide that is adjacent to X₀ is capped with P¹, wherein P¹ is selected from H,

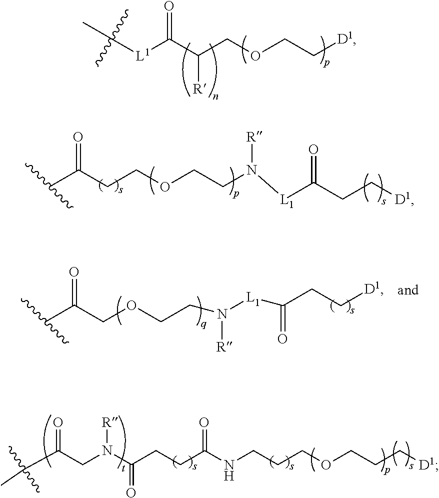

D¹ is selected from H, CH₃, and C(O)OH;
L¹ is absent or selected from

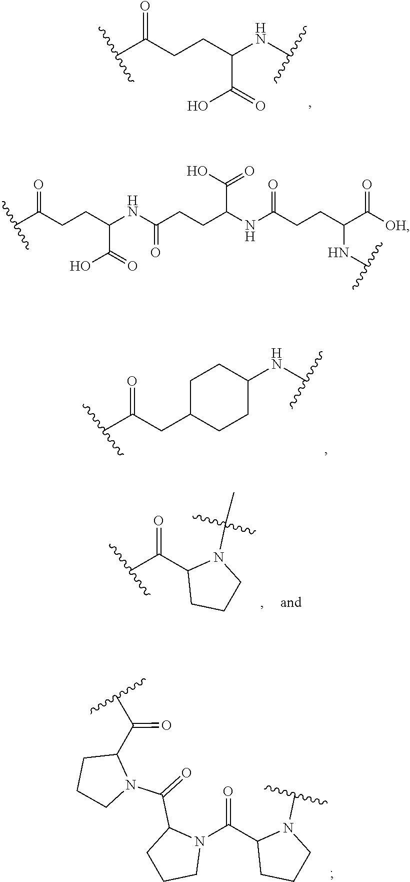 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the amino group of L¹ connects to the carbonyl group of P¹ to form an amide bond;
each n and q are independently an integer from 0 to 16;
each p is independently an integer from 0 to 24;
each s is independently an integer from 0 to 16;
each t is independently 0, 1, 2, 3, 4, 5, or 6;
each R′ is independently selected from H, C(O)OH, (CH₂)OH, and NHAc; and
each R″ is independently selected from H and CH₃.

In another embodiment of the above aspects, X₀ is absent or X₀ is selected from Gly, Met, D-Ala, Ala, Nle, and Nva. In still another embodiment, X₀ is absent.

In an embodiment of the above aspects, X₁ is selected from Trp or 7Me-Trp. In another embodiment, X₁ is Trp. If X₀ is absent, then X₁ can be substituted with an N-terminus group selected from P¹.

In an embodiment of the above aspects, X₂ is selected from Thr, D-Ala, Ala, alpha-Me-Thr, Lys, and NMe-Thr. In another embodiment, X₂ is selected from the group consisting of Thr, alpha-Me-Thr, and NMe-Thr. In yet another embodiment, X₂ is Thr.

In still another embodiment of the above aspects, X₃ is selected from Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, AIlo-Ile, Lys(C12), Lys(C14), Lys(C16), alpha-Me-Ile, and NMe-tBuAla. In an embodiment, X₃ is selected from the group consisting of Ile, AIlo-Ile, alpha-Me-Ile, and NMe-tBuAla. In another embodiment, X₃ is selected from the group consisting of D-Ala, Ala, t-Bu-Ala, and NMe-tBuAla. In yet another embodiment, X₃ is Ile or NMe-tBuAla. In still another embodiment, X₃ is lie. In an embodiment, X₃ is NMe-tBuAla.

In another embodiment of the above aspects, the linker between Y₁ and Y₂ is selected from a bond, C₁₋₆ alkylene, and

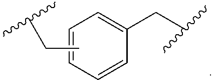

In yet another embodiment of the above aspects, X₄ is selected from Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, and NMe-Asn. In still another embodiment of the above aspects, X₄ is selected from Asn, 3-(4-piperidinyl)-Ala, and Pip(CH₂CO₂H)Ala. In an embodiment, X₄ is 3-(4-piperidinyl)-Ala or Pip(CH₂CO₂H)Ala. In another embodiment, X₄ is Pip(CH₂CO₂X)Ala, wherein X is a pharmaceutically acceptable cation (e.g., to form a pharmaceutically acceptable salt).

In another embodiment of the above aspects, X₅ is selected from Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser. In another embodiment of the above aspects, X₅ is Asn or NMe-Asn. In yet another embodiment, X₅ is Asn. In still another embodiment, X₅ is NMe-Asn.

In an embodiment of the above aspects, X₆ is selected from Trp, 4CF₃-Phe, 1Me-Trp, 7Aza-Trp, BIP, 2Nal, 1Nal, aMe-Trp, D-Ala, Ala, 4F-Phe, 5F-Trp, 5Ome-Trp, Asn, 5OH-Trp, 7Me-Trp, 7MeO-Trp, 7Cl-Trp, and NMe-Trp. In another embodiment, X₆ is selected from the group consisting of Trp, 1Me-Trp, 7Aza-Trp, 2Nal, 1Nal, alpha-Me-Trp, 5F-Trp, 5MeO-Trp, 5OH-Trp, 7Me-Trp, 7MeO-Trp, 7Cl-Trp, and NMe-Trp. In yet another embodiment of the above aspects, X₆ is selected from Trp, 2Nal, and 1 Nal. In still another embodiment, X₆ is Trp. In an embodiment, X₆ is 2Nal.

In another embodiment of the above aspects, X₇ is selected from 3Pya, 4Pya, Lys(Me)3, His, Ala, D-Ala, Gln, Lys, Glu, Arg, Orn, NMe-His, and Ser. In yet another embodiment, X₇ is His or Lys. In still another embodiment, X₇ is His. In an embodiment, X₇ is Lys.

In another embodiment of the above aspects, X₈ is selected from Asp, Asn, NMe-Asp, and alpha-Me-Asp. In yet another embodiment, X₈ is Asp.

In still another embodiment of the above aspects, X₉ is Trp.

In an embodiment of the above aspects, X₁₀ is Pro.

In another embodiment, P² is -L²-Chelator.

In yet another embodiment, P² is

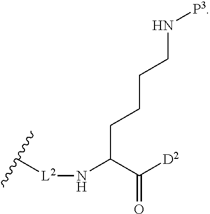

In still another embodiment, P³ is -L³-Chelator.

In an embodiment P³ is

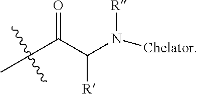

In another embodiment, Chelator is selected from a Chelator in Table C.

In another aspect, provided herein is a cyclic peptide of Formula I:

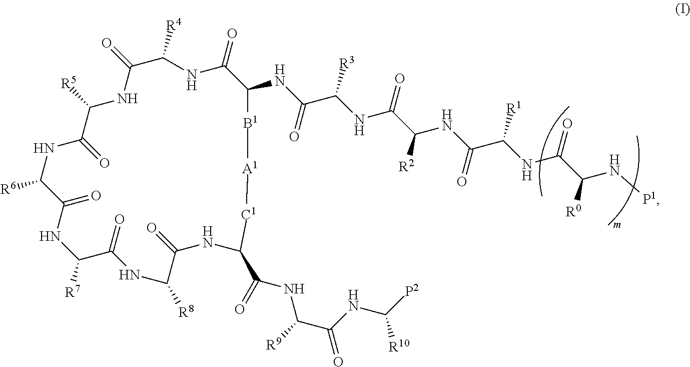

or a pharmaceutically acceptable salt thereof,
wherein:
P¹ is selected from: H,

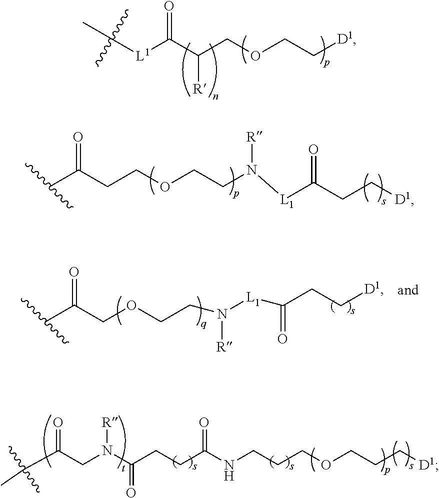

D¹ is selected from H, CH₃, and C(O)OH;
L¹ is absent or selected from

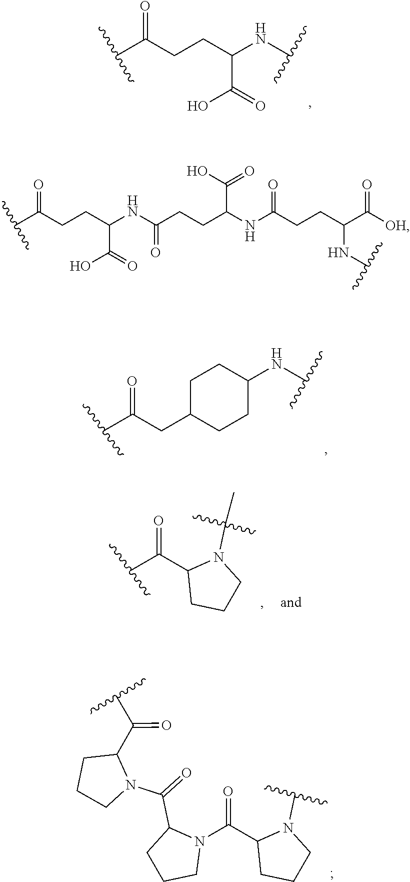 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the amino group of L¹ connects to the carbonyl group of P¹ to form an amide bond;
P² is -L²-Chelator or

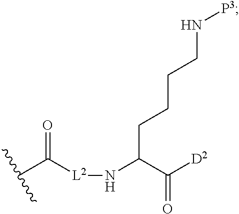

D² is OH or NH₂;
L² is absent or selected from:

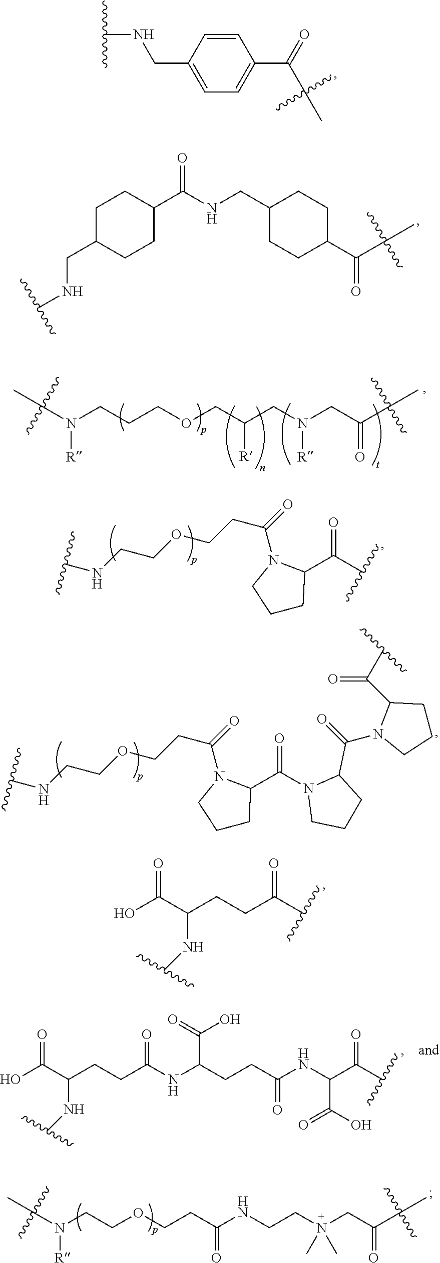

wherein the amino group of L² connects to the carbonyl group of P² or Chelator to form an amide bond;
P³ is selected from -L³-Chelator,

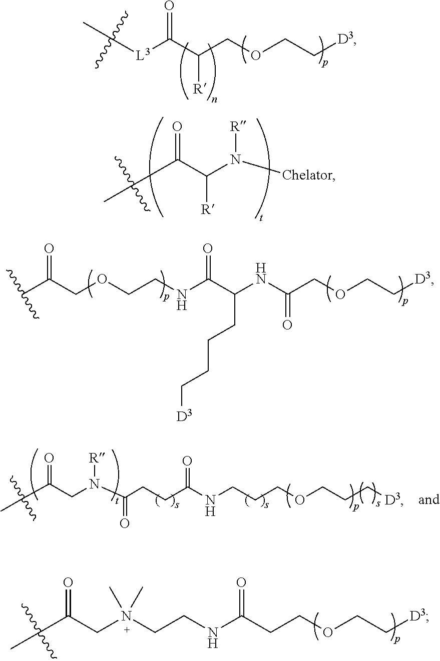

L³ is absent or independently selected from

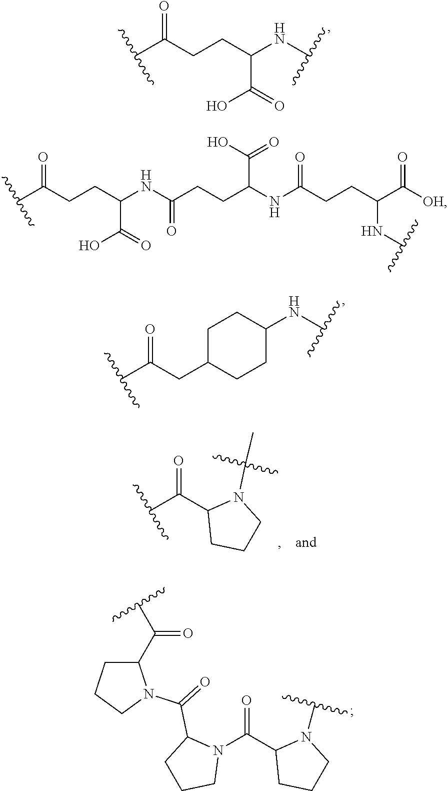

wherein the carbonyl group of L³ connects to an amine group of P² to form an amide bond;
D³ is —NR″-Chelator;
R⁰ is an amino acid side chain of a natural amino acid or an amino acid side chain of an unnatural amino acid;
R¹ is an amino acid side chain of a natural amino acid or an amino acid side chain of an unnatural amino acid;
R² is an amino acid side chain of a natural amino acid or an amino acid side chain of an unnatural amino acid;
R³ is an amino acid side chain of a natural amino acid or an amino acid side chain of an unnatural amino acid;
B¹ is C₁₋₆ alkylene;
C¹ is C₁₋₆ alkylene;
A¹ is selected from:

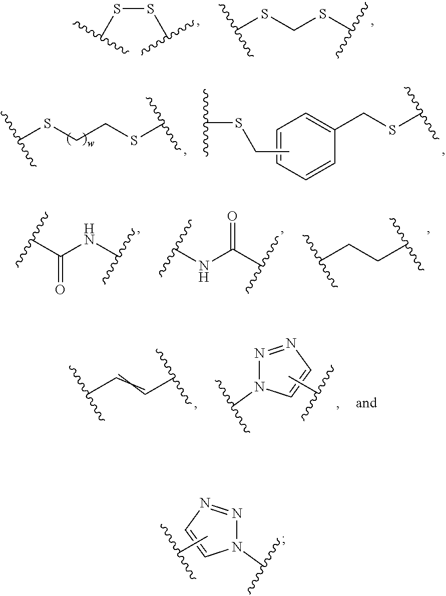

wherein w is selected from 1, 2, or 3;
R⁴ is an amino acid side chain of a natural amino acid or an amino acid side chain of an unnatural amino acid, both of which are optionally substituted with CH₂C(O)OH or C(O)(CH₂CH₂O)ₚ(CH₂)₂N(CH₃)₃⁺;
R⁵ is an amino acid side chain of a natural amino acid or an amino acid side chain of an unnatural amino acid;
R⁶ is an amino acid side chain of a natural amino acid or an amino acid side chain of an unnatural amino acid;
R⁷ is selected from:
(i) an amino acid side chain of a natural amino acid,
(ii) an amino acid side chain of an unnatural amino acid, or
(iii) selected from the group consisting of:

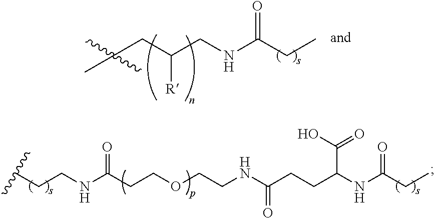

R⁸ is an amino acid side chain of a natural amino acid or an amino acid side chain of an unnatural amino acid;
R⁹ is an amino acid side chain of a natural amino acid or an amino acid side chain of an unnatural amino acid;
R¹⁰ is an amino acid side chain of a natural amino acid or an amino acid side chain of an unnatural amino acid;
m is 0 or 1;
each n, q, and u are independently an integer from 0 to 16;
each p is independently an integer from 0 to 24;
each s is independently an integer from 0 to 16;
each t is independently 1, 2, 3, 4, 5, or 6;
each R′ is independently selected from H, C(O)OH, (CH₂)OH, and NHAc; and
each R″ is independently selected from H and CH₃.

In an embodiment, each nitrogen atom and alpha-carbon atom on the peptide backbone is optionally substituted with methyl;
wherein when a variable group R⁰, R¹, R², R³, R⁴, R⁵, R⁶, R⁷, R⁸, R⁹, or R¹⁰ is defined as the amino acid side chain of a cyclic amino acid, the corresponding amino acid nitrogen of the peptide backbone of Formula I forms part of the cyclic group; and
and wherein the cyclic peptide optionally comprises a radionuclide.

In an embodiment, the cyclic peptide of Formula I is a cyclic peptide of Formula I′:

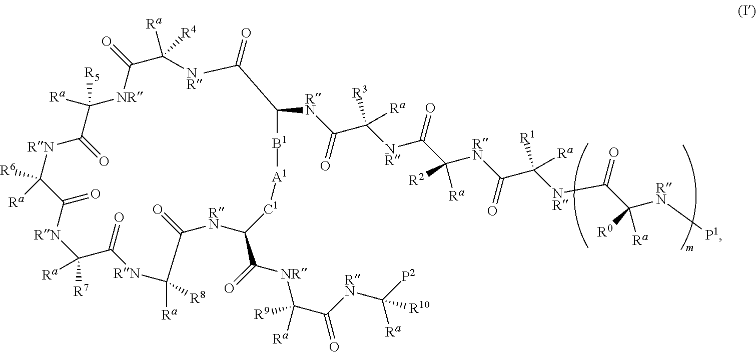

or a pharmaceutically acceptable salt thereof.

In yet another aspect, provided herein is a cyclic peptide of Formula B:

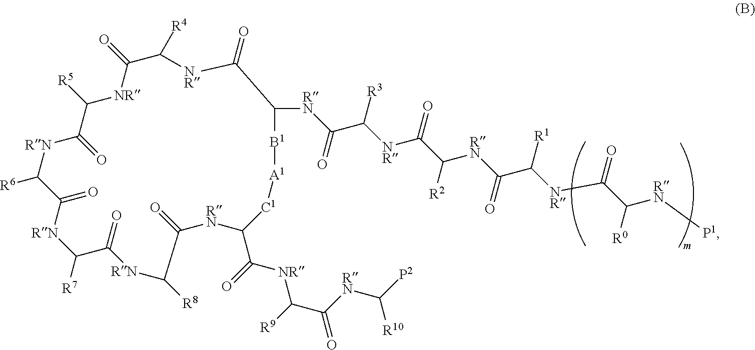

or a pharmaceutically acceptable salt thereof,
wherein
P¹ is selected from: H,

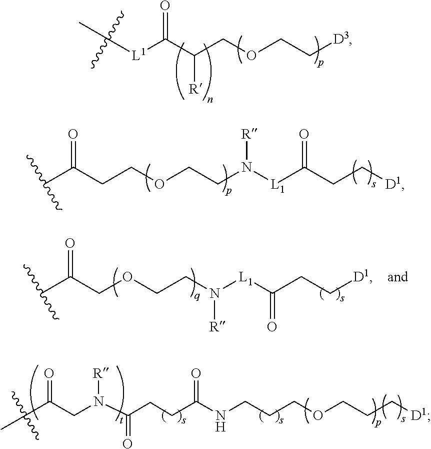

D¹ is selected from H, CH₃, and C(O)OH;
L¹ is absent or selected from

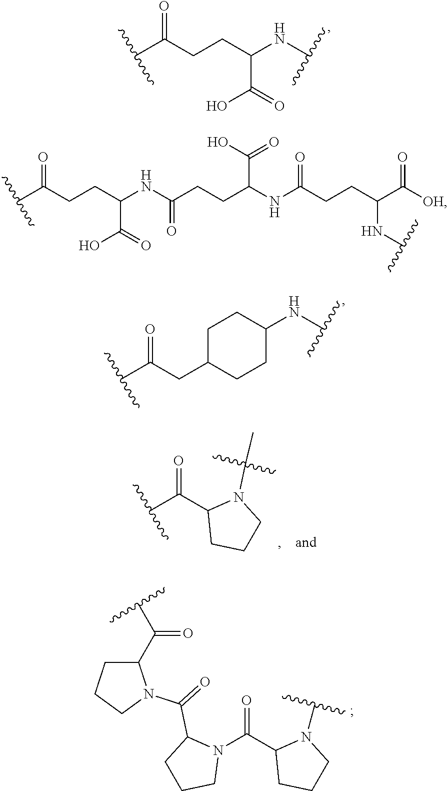

wherein the amino group of L¹ connects to the carbonyl group of P¹ to form an amide bond;
P² is -L²-Chelator or

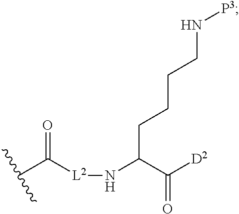

D² is OH or NH₂;
L² is absent or selected from:

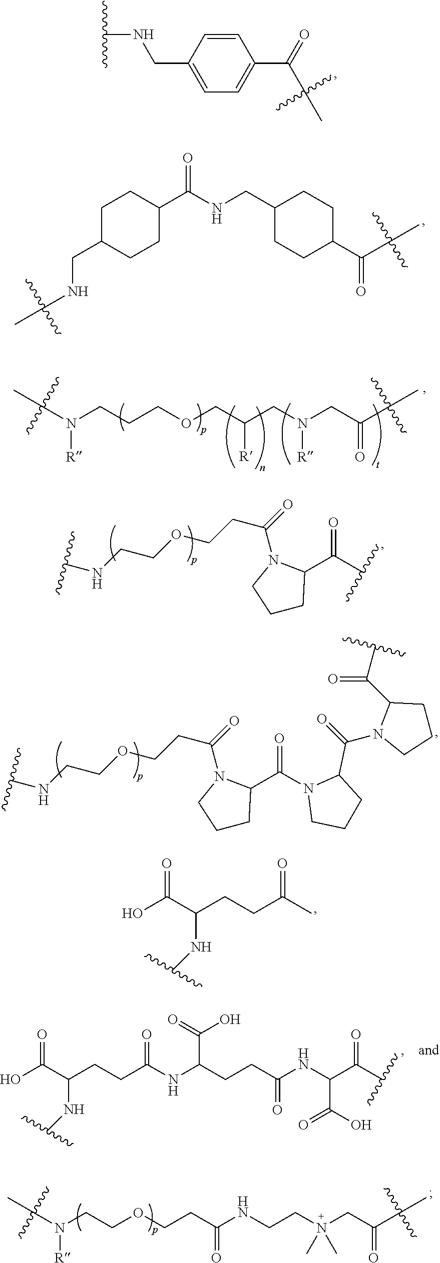

wherein the amino group of L² connects to the carbonyl group of P² or Chelator to form an amide bond;
P³ is selected from -L³-Chelator,

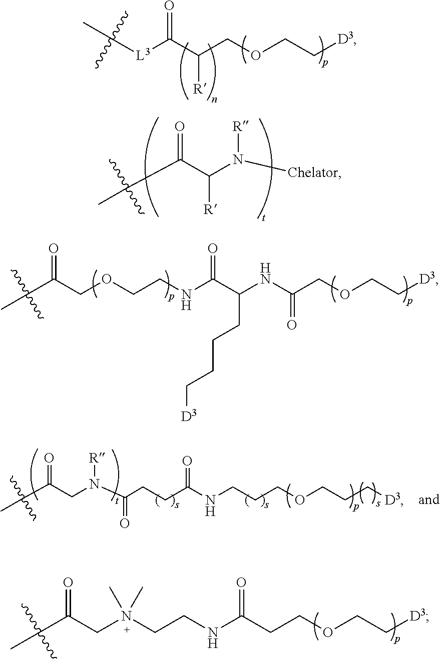

L³ is absent or independently selected from

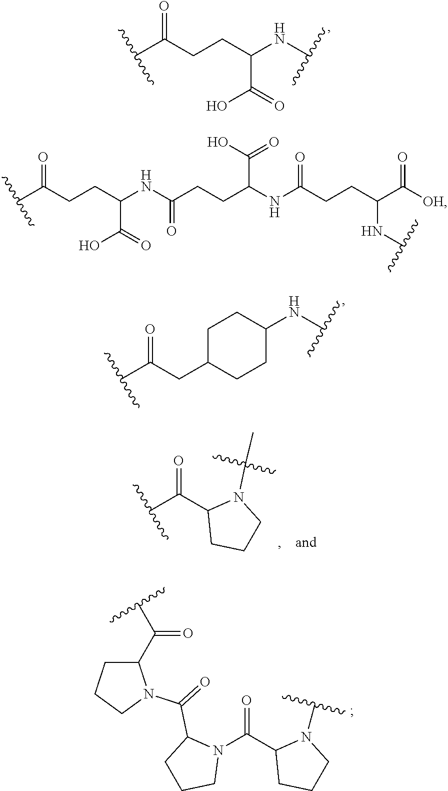

wherein the carbonyl group of L³ connects to an amine group of P² to form an amide bond;
D³ is —NR″-Chelator;
R⁰ is an amino acid side chain of a natural amino acid or an amino acid side chain of an unnatural amino acid;
R¹ is an amino acid side chain of a natural amino acid or an amino acid side chain of an unnatural amino acid;
R² is an amino acid side chain of a natural amino acid or an amino acid side chain of an unnatural amino acid;
R³ is an amino acid side chain of a natural amino acid or an amino acid side chain of an unnatural amino acid;
B¹ is C₁₋₆ alkylene;
CI is C₁₋₆ alkylene;
A¹ is selected from:

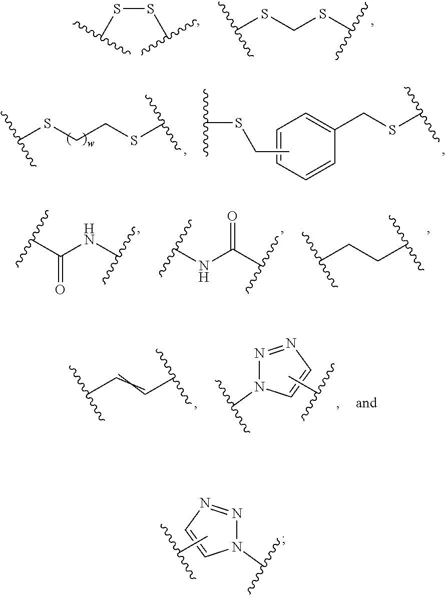

wherein w is selected from 1, 2, or 3;
R⁴ is an amino acid side chain of a natural amino acid or an amino acid side chain of an unnatural amino acid, both of which are optionally substituted with CH₂C(O)OH or C(O)(CH₂CH₂O)ₚ(CH₂)₂N(CH₃)₃⁺;
R⁵ is an amino acid side chain of a natural amino acid or an amino acid side chain of an unnatural amino acid;
R⁶ is an amino acid side chain of a natural amino acid or an amino acid side chain of an unnatural amino acid;
R⁷ is selected from:
(i) an amino acid side chain of a natural amino acid,
(ii) an amino acid side chain of an unnatural amino acid, or
(iii) selected from the group consisting of:

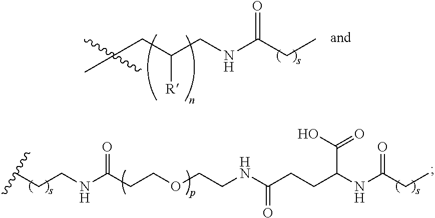

R⁸ is an amino acid side chain of a natural amino acid or an amino acid side chain of an unnatural amino acid;
R⁹ is an amino acid side chain of a natural amino acid or an amino acid side chain of an unnatural amino acid;
R¹⁰ is an amino acid side chain of a natural amino acid or an amino acid side chain of an unnatural amino acid;
m is 0 or 1;
each n, q, and u are independently an integer from 0 to 16;
each p is independently an integer from 0 to 24;
each s is independently an integer from 0 to 16;
each t is independently 1, 2, 3, 4, 5, or 6;
each R′ is independently selected from H, C(O)OH, (CH₂)OH, and NHAc; and
each R″ is independently selected from H and CH₃;
wherein when a variable group R⁰, R¹, R², R³, R⁴, R⁵, R⁶, R⁷, R⁸, R⁹, or R¹⁰ is defined as the amino acid side chain of a cyclic amino acid, the corresponding amino acid nitrogen of the peptide backbone of Formula B forms part of the cyclic group; and
and wherein the cyclic peptide optionally comprises a radionuclide.

In an embodiment, the cyclic peptide of Formula B is a cyclic peptide of Formula Bi:

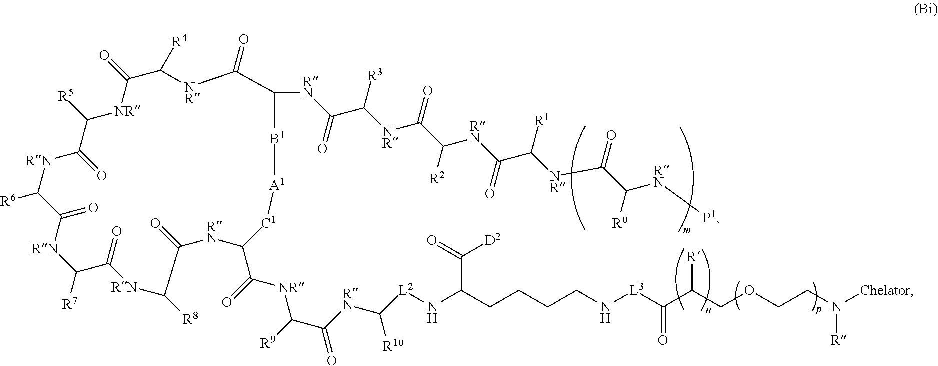

or a pharmaceutically acceptable salt thereof.

In another embodiment, the cyclic peptide of Formula B is a cyclic peptide of Formula Bii:

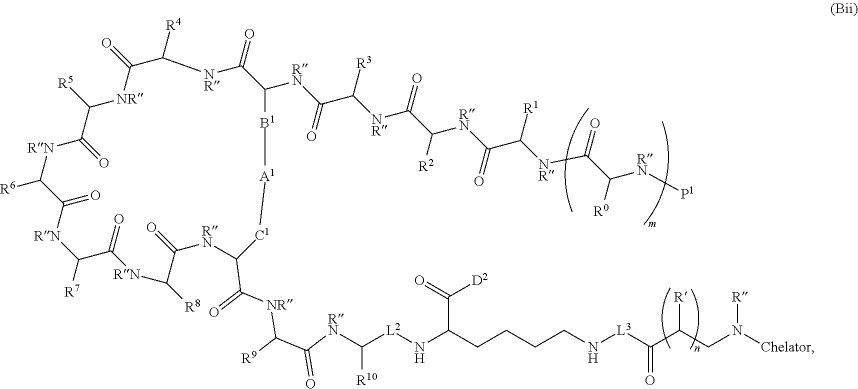

or a pharmaceutically acceptable salt thereof.

In another embodiment, the cyclic peptide of Formula B is a cyclic peptide of Formula Biii:

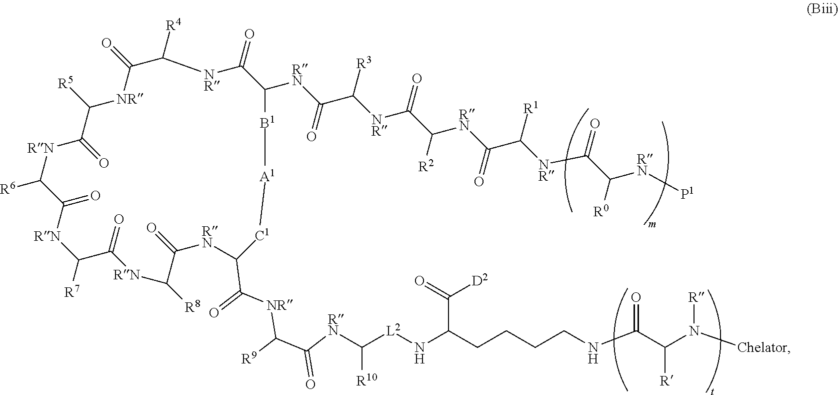

or a pharmaceutically acceptable salt thereof.

In an embodiment, the cyclic peptide of Formula B is a cyclic peptide of Formula Ia:

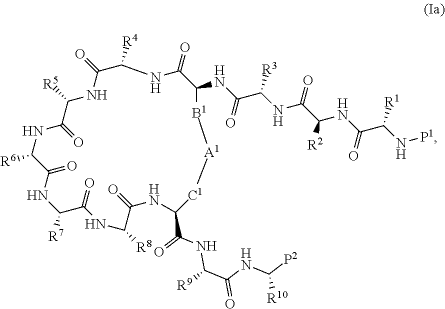

or a pharmaceutically acceptable salt thereof.

In another embodiment, the cyclic peptide of Formula B is a cyclic peptide of Formula Ib:

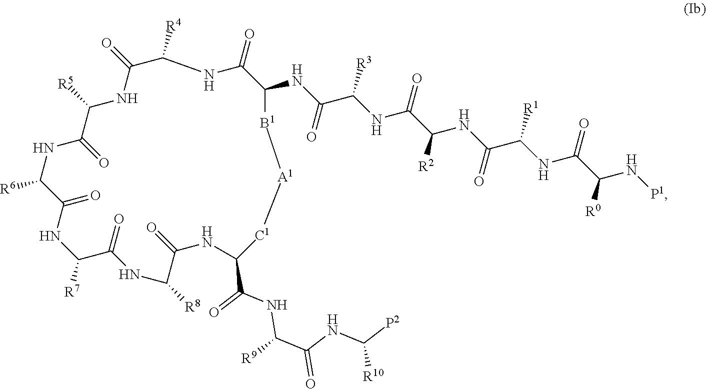

or a pharmaceutically acceptable salt thereof.

In yet another embodiment, P¹ is selected from Ac,

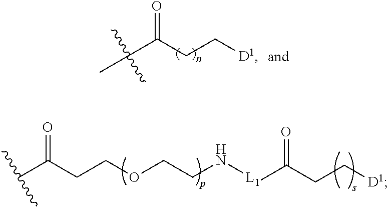

wherein:
D¹ is CH₃ or C(O)OH;
L¹ is absent or

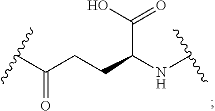 [SMILES: *N[C@@H](CCC(*)=O)C(=O)O]

n and s are independently an integer from 2 to 15; and
p is 8, 9, 10, 11, or 12.

In still another embodiment, P¹ is Ac or CH₃(OCH₂CH₂)₈₋₁₆C(O).

In an embodiment, P¹ is selected from H, Ac,

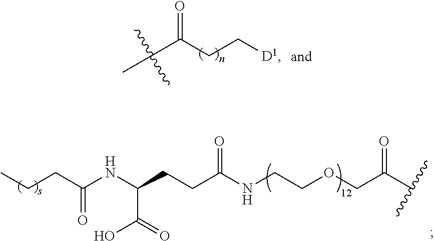

wherein:
n and s are independently 9, 10, 11, 12, or 13; and
D¹ is CH₃ or C(O)OH.

In another embodiment, P² is -L²-Chelator. In yet another embodiment, P² is

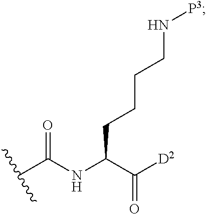

P³ is selected from -L³-Chelator,

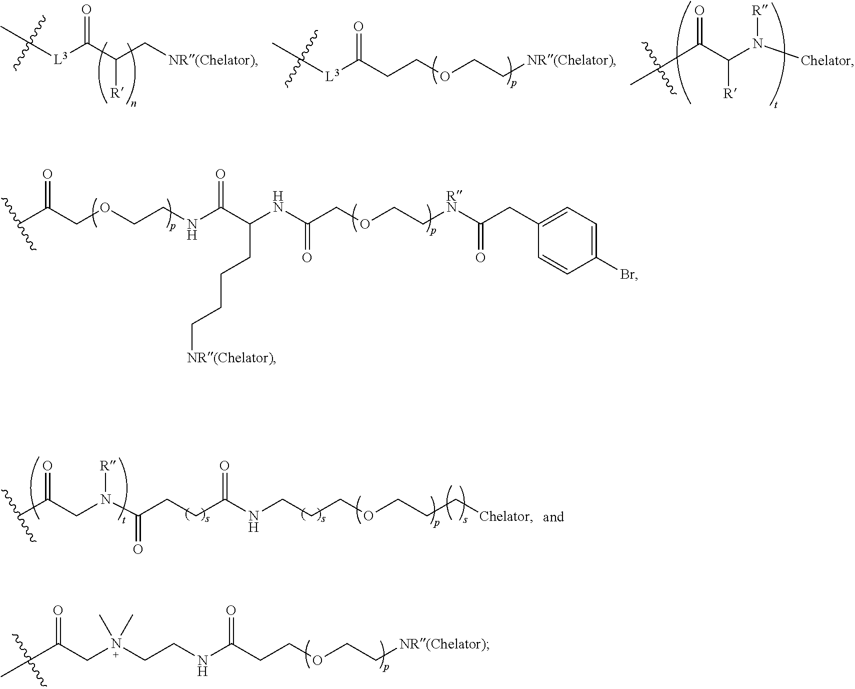

L³ is absent or independently selected from

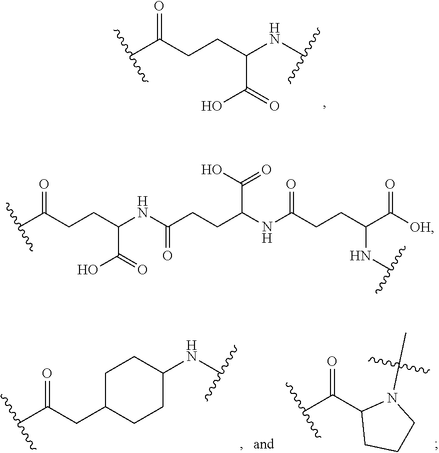 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1*]

In yet another embodiment, P² is
P³ is selected from Chelator,

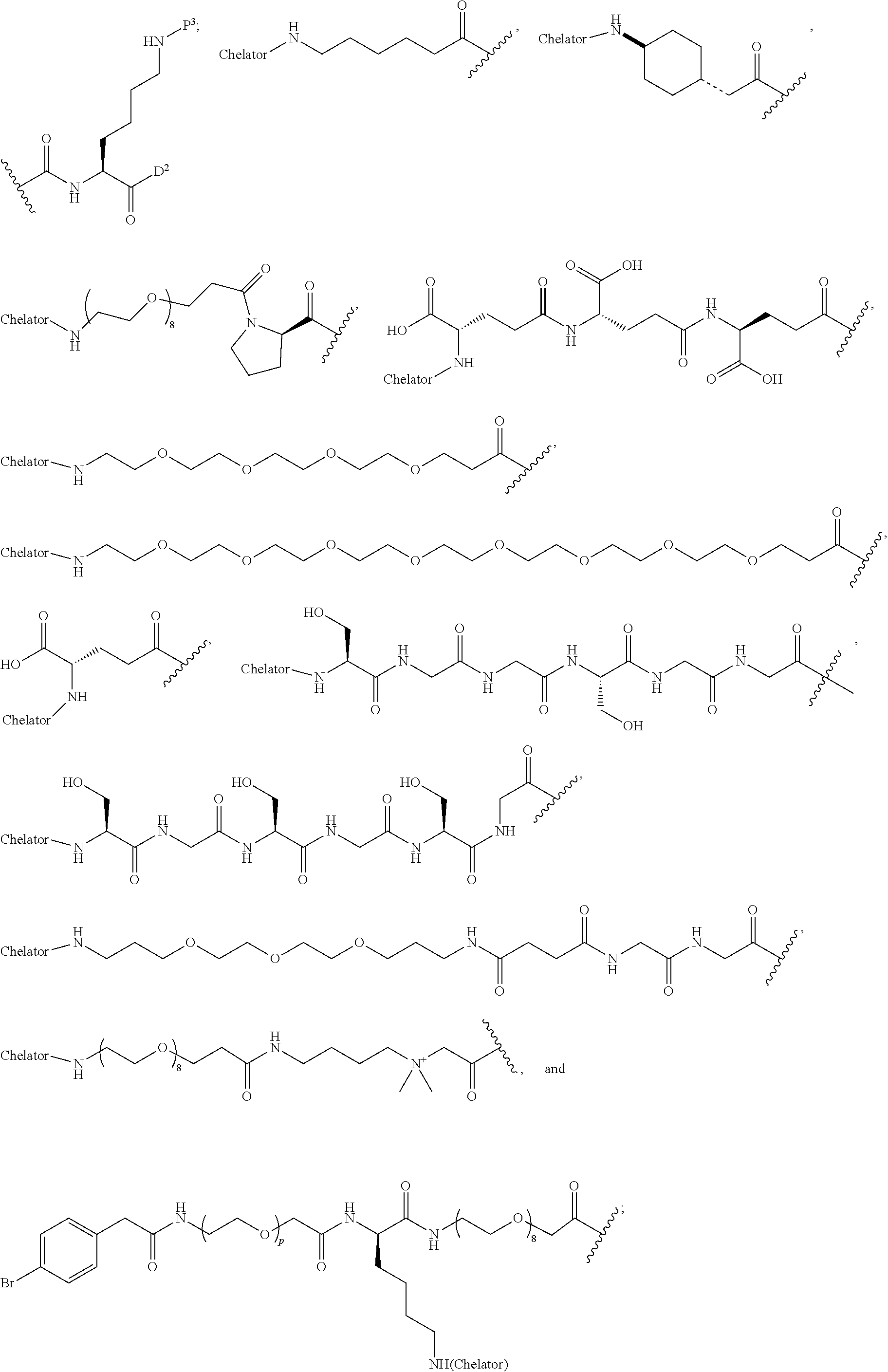

and
p is 8, 12, or 24.

In yet another embodiment, P² is
P³ is selected from DOTA,

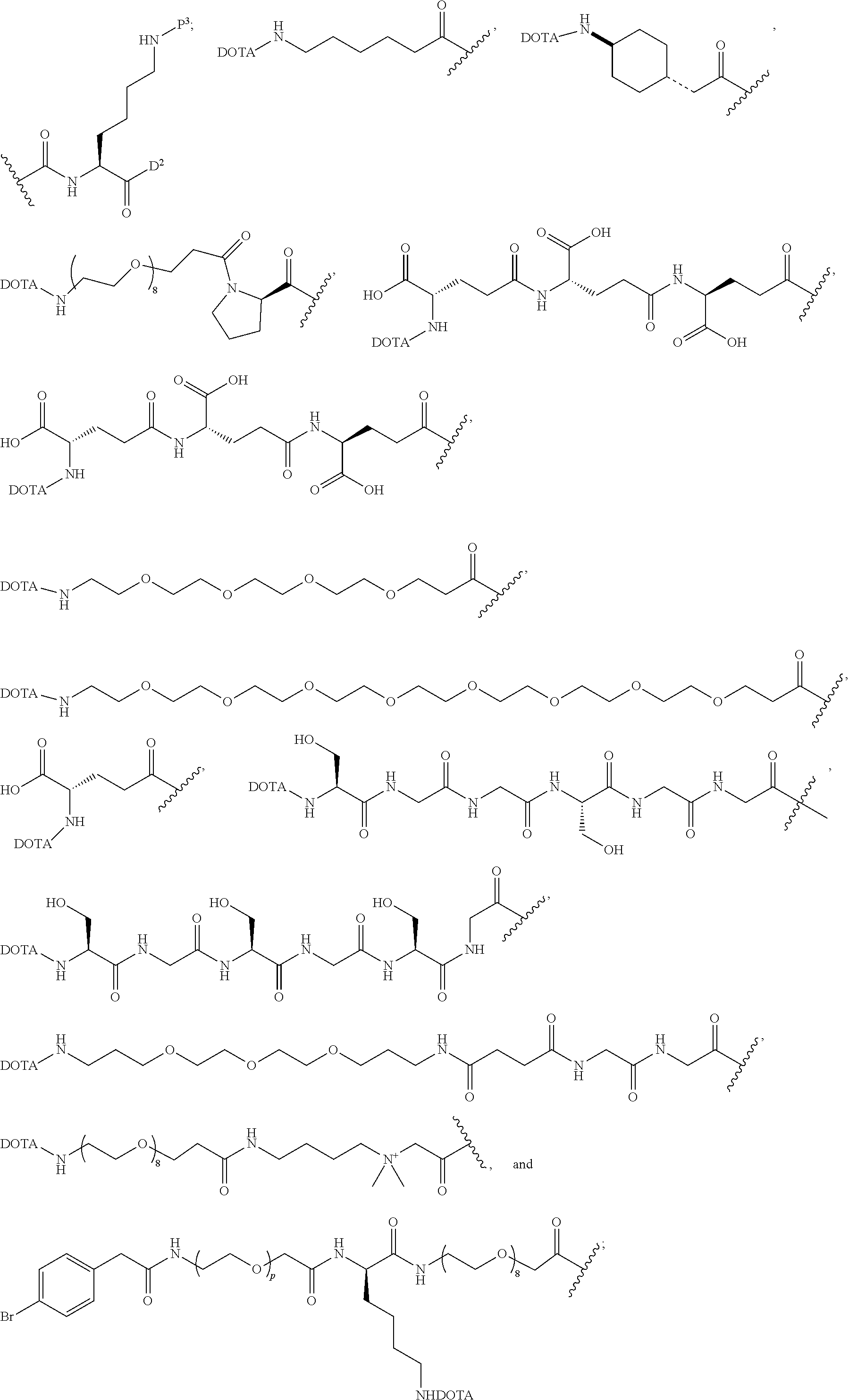

and
p is 8, 12, or 24.

In still another embodiment, P³ is -L³-Chelator.

In an embodiment P³ is

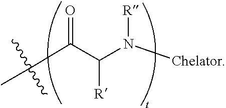

In another embodiment, D³ is —NR″-Chelator.

In another aspect, provided herein is a cyclic peptide of Formula I, wherein D³ is

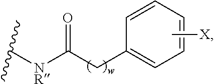

wherein w is selected from 1, 2, or 3; and the other variables are defined herein.

In an embodiment, P³ is Ac or

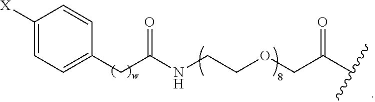

In still another embodiment, the cyclic peptide of Formula B is a cyclic peptide of Formula II:

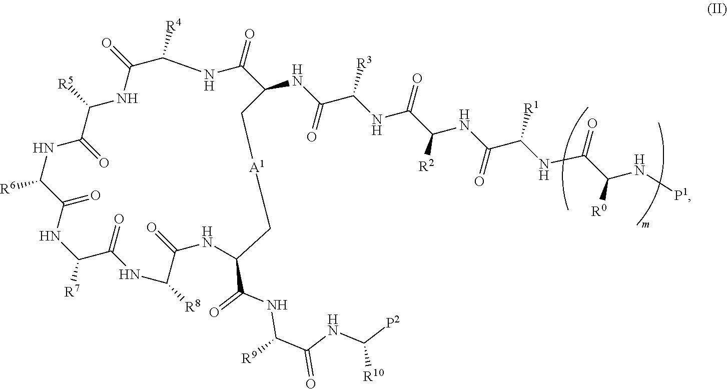

or a pharmaceutically acceptable salt thereof.

In an embodiment, the cyclic peptide of Formula B is a cyclic peptide of Formula III:

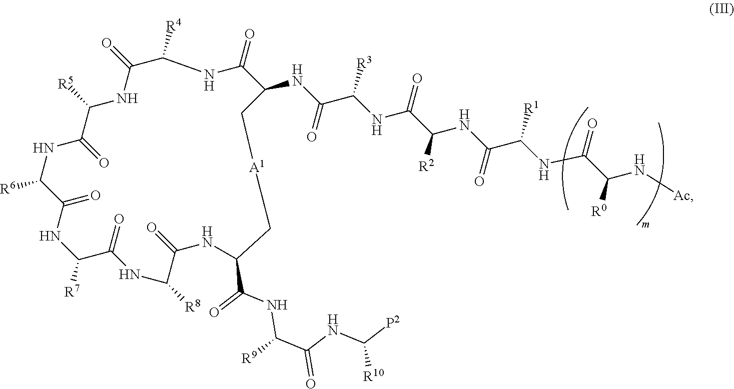

or a pharmaceutically acceptable salt thereof.

In yet another embodiment,
m is 0;
P¹ is selected from H, Ac,

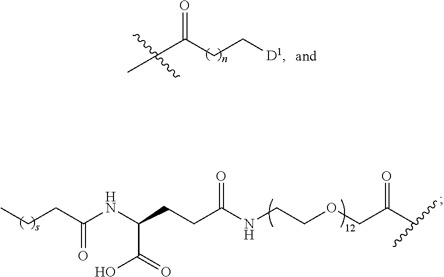

wherein:
n and s are each independently 9, 10, 11, 12, or 13;
P² is

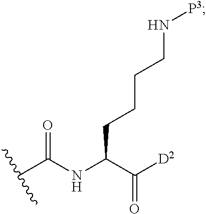

P³ is selected from DOTA,

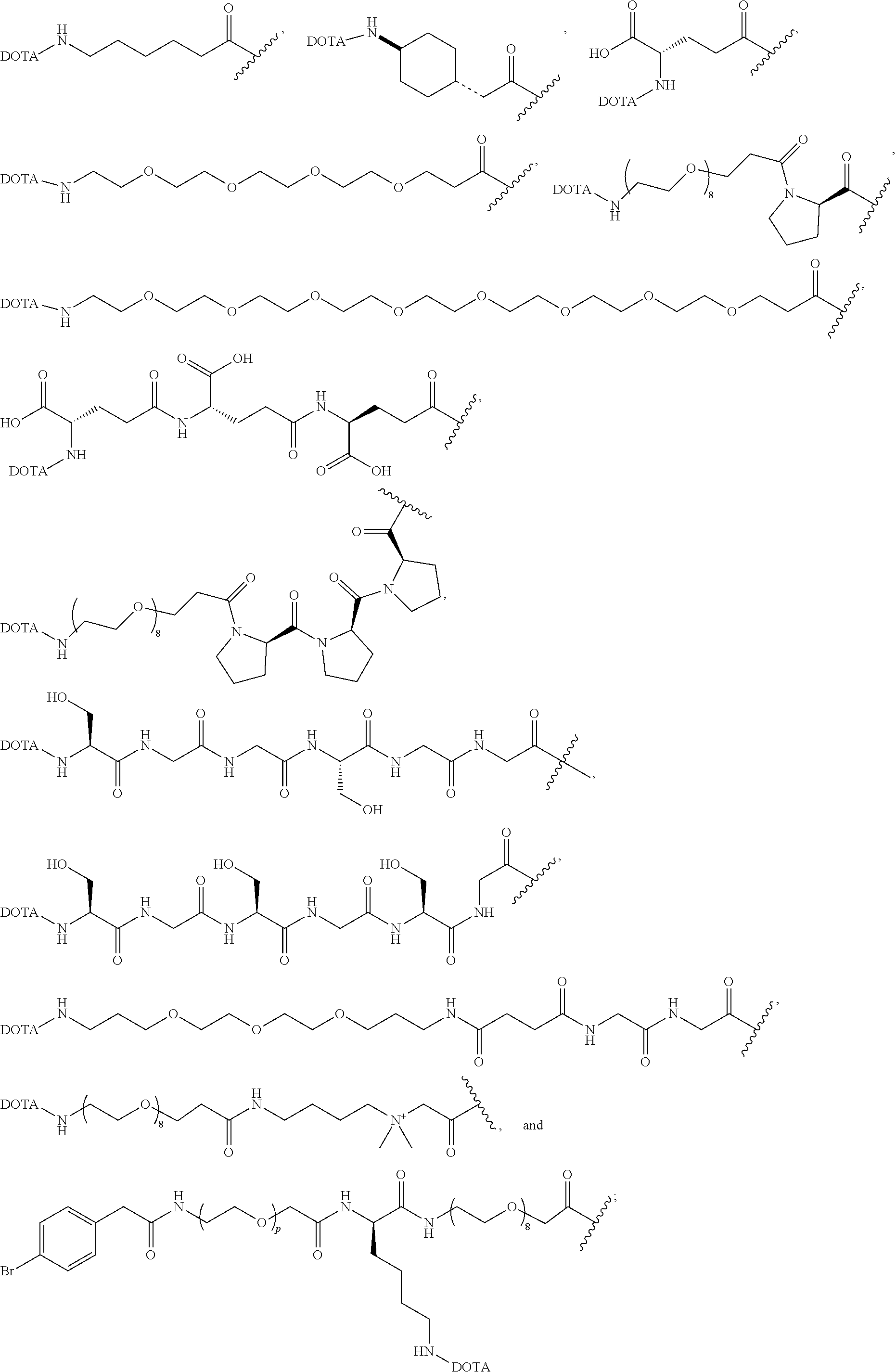

p is 8, 12, or 24;
D¹ is CH₃ or C(O)OH;
R¹ is selected from the group consisting of an amino acid side chain of Trp, 2Nal, 1Nal, 4CF₃-Phe, 7Aza-Trp, 1Me-Trp, 5OH-Trp, BIP, 5Ome-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is selected from the group consisting of an amino acid side chain of Thr, D-Ala, Ala, alpha-Me-Thr, Lys, and NMe-Thr;
R³ is selected from the group consisting of an amino acid side chain of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, AIlo-Ile, Lys(C12), Lys(C14), Lys(C16), alpha-Me-Ile, and NMe-tBuAla;
A¹ is selected from the group consisting of:

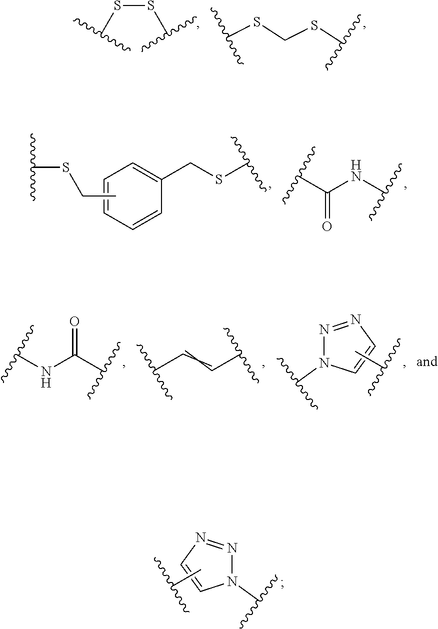

R⁴ is selected from the group consisting of an amino acid side chain of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, NMe-Asn, Pip(CH₂CO₂H)Ala, and Pip(PegNMe₃)Ala;
R⁵ is selected from the group consisting of an amino acid side chain of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is selected from the group consisting of an amino acid side chain of Trp, 4CF₃-Phe, 1Me-Trp, 7Aza-Trp, BIP, 2Nal, 1Nal, alpha-Me-Trp, D-Ala, Ala, 4F-Phe, 5F-Trp, 5Ome-Trp, Asn, 5OH-Trp, 7Me-Trp, 7MeO-Trp, 7Cl-Trp, and NMe-Trp;
R⁷ is:
(i) selected from the group consisting of an amino acid side chain of 3Pya, 4Pya, Lys(Me)3, His, Ala, D-Ala, Gln, Lys, Glu, Arg, Orn, NMe-His, and Ser; or
(ii) selected from the group consisting of:

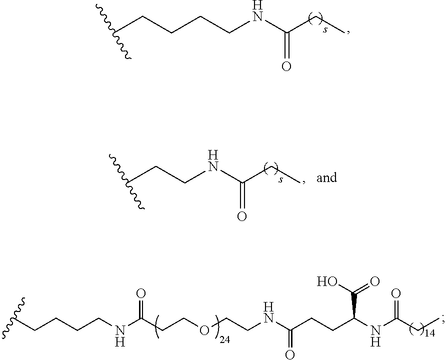

each s is independently 3, 5, 10, 12, or 14;
R⁸ is selected from the group consisting of an amino acid side chain of Asp, D-Ala, Ala, Asn, Thr, NMe-Asp, and alpha-Me-Asp;
R⁹ is selected from the group consisting of an amino acid side chain of Trp, 7Aza-Trp, 1Me-Trp, D-Ala, Ala, 4F-Phe, 1Nal, 2Nal, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, D-Ala, Ala, alpha-Me-Pro, trans4Fluoro-Pro, cis4Fluoro-Pro, trans40H-Pro, cis4OH-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACI, and 3Me2-Aze.

In another embodiment,
m is 1;
P¹ is selected from Ac,

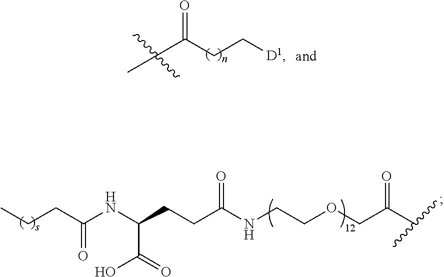

wherein:
n and s are each independently 9, 10, 11, 12, or 13;
P² is

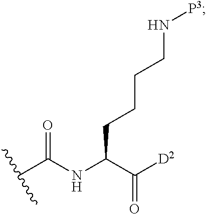

P³ is selected from DOTA,

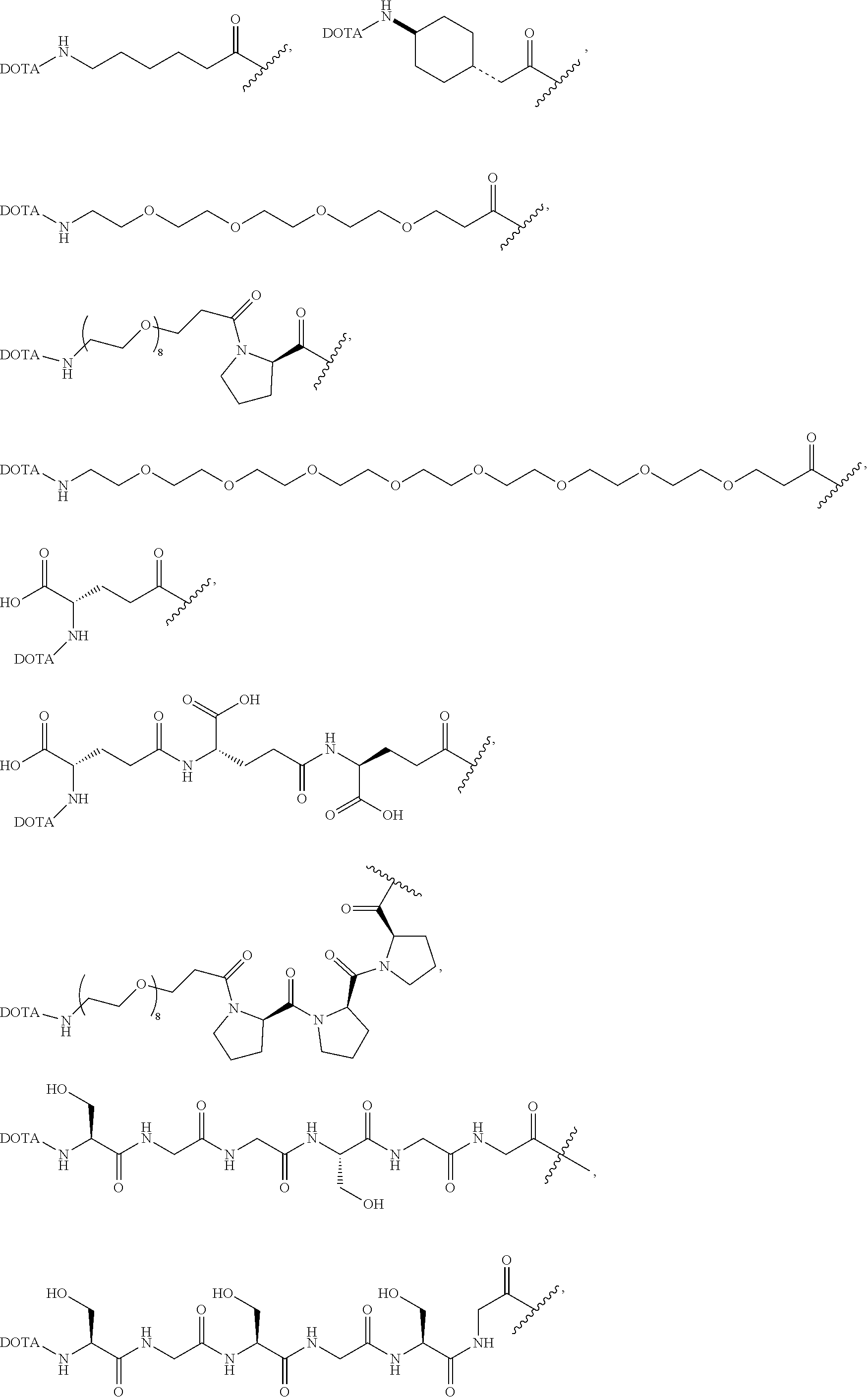

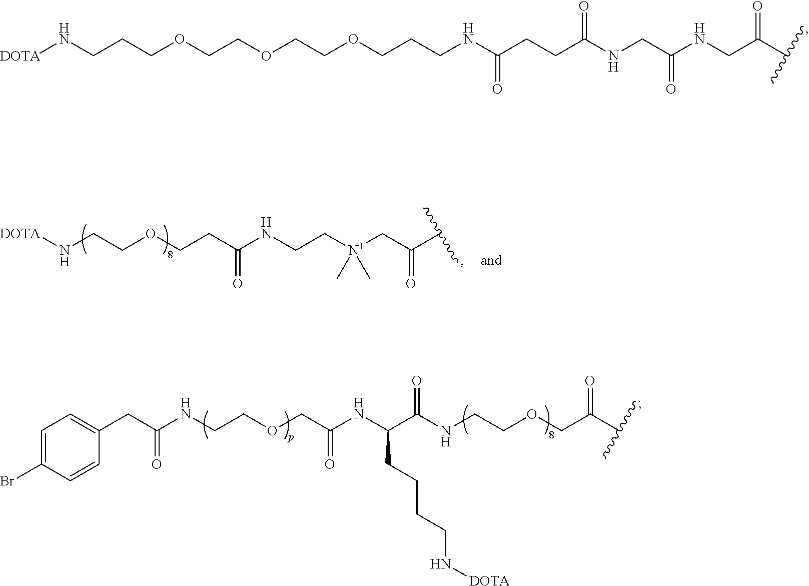

wherein
p is 8, 12, or 24;
D¹ is CH₃ or C(O)OH;
R⁰ is selected from the group consisting of an amino acid side chain of Gly, Met, D-Ala, Ala, Nle, and Nva;
R¹ is selected from the group consisting of an amino acid side chain of Trp, 2Nal, 1Nal, 4CF₃-Phe, 7Aza-Trp, 1Me-Trp, 5OH-Trp, BIP, 5Ome-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is selected from the group consisting of an amino acid side chain of Thr, D-Ala, Ala, alpha-Me-Thr, Lys, and NMe-Thr;
R³ is selected from the group consisting of an amino acid side chain of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, AIlo-Ile, Lys(C12), Lys(C14), Lys(C16), alpha-Me-Ile, and NMe-tBuAla;
A¹ is selected from the group consisting of

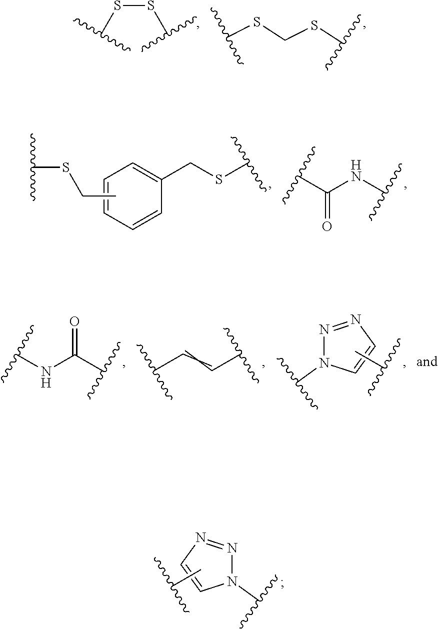

R⁴ is selected from the group consisting of an amino acid side chain of Asn, D-Ala, Ala, DAB-4-NHCOC₅H₁₁, DAB-4-NHCOC₇H₁₅, Asp, SerLys, 3-(4-piperidinyl)-Ala, 3-(1-morpholinyl)-Ala, 3Pya, 4Pya, Glu, and NMe-Asn;
R⁵ is selected from the group consisting of an amino acid side chain Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is selected from the group consisting of an amino acid side chain of Trp, 4CF₃-Phe, 1Me-Trp, 7Aza-Trp, BIP, 2Nal, 1Nal, alpha-Me-Trp, D-Ala, Ala, 4F-Phe, 5F-Trp, 5Ome-Trp, Asn, 5OH-Trp, 7Me-Trp, 7MeO-Trp, 7Cl-Trp, and NMe-Trp;
R⁷ is selected from the group consisting of an amino acid side chain of 3Pya, 4Pya, Lys(Me)3, His, Ala, D-Ala, Gln, Lys, Glu, Arg, Orn, NMe-His, and Ser; or
R⁷ is selected from the group consisting of

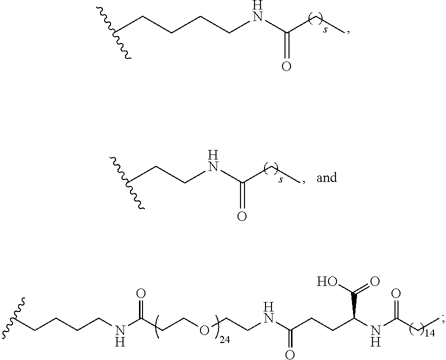

each s is independently 3, 5, 10, 12, or 14;
R⁸ is selected from the group consisting of an amino acid side chain of Asp, D-Ala, Ala, Asn, Thr, NMe-Asp, and alpha-Me-Asp;
R⁹ is selected from the group consisting of an amino acid side chain of Trp, 7Aza-Trp, 1Me-Trp, D-Ala, Ala, 4F-Phe, 1Nal, 2Nal, 5F-Trp, 5Ome-Trp, alpha-Me-Trp, 7Cl-Trp, 5OH-Trp, 7Me-Trp, 7MeO-Trp, and NMe-Trp; and
R¹⁰ is from the group consisting of an amino acid side chain of Pro, D-Ala, Ala, alpha-Me-Pro, trans4Fluoro-Pro, cis4Fluoro-Pro, trans40H-Pro, cis4OH-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACI, and 3Me2-Aze.

In an embodiment, m is 0 or 1;
P¹ is selected from the group consisting of H, Ac,

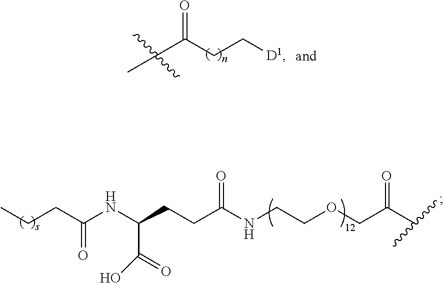

n and s are each independently 9, 10, 11, 12, or 13;
D¹ is CH₃ or C(O)OH;
P² is

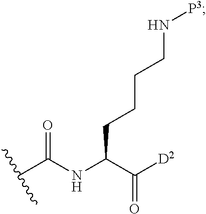

D² is OH or NH₂;
P³ is selected from the group consisting of Chelator,

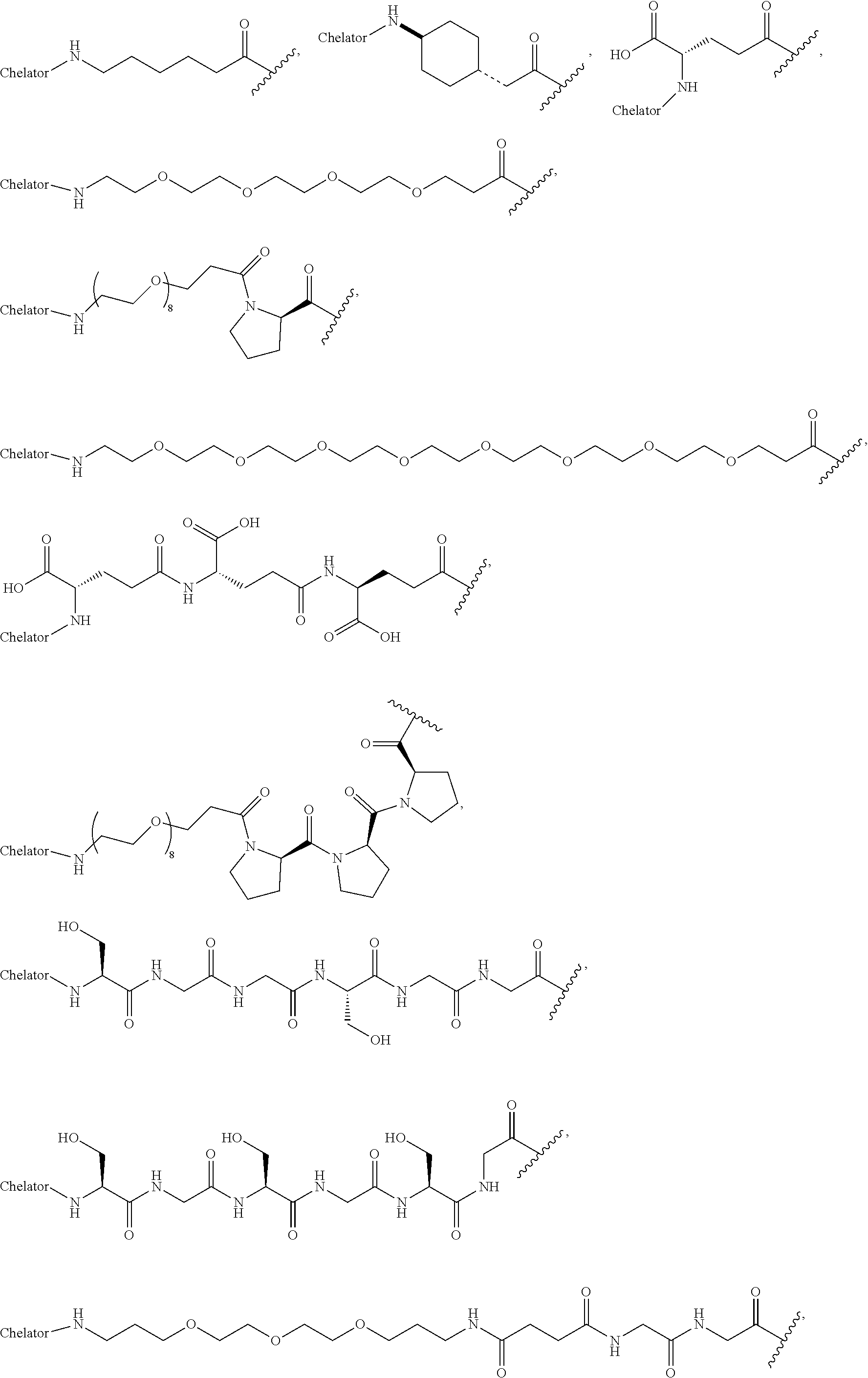

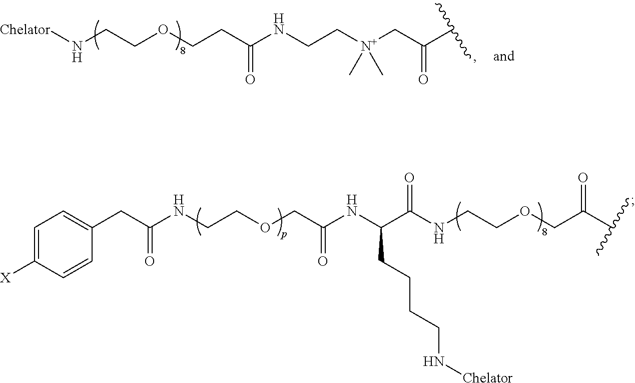

p is an integer selected from 6-24;
X is halogen;
R⁰ is selected from the group consisting of an amino acid side chain of Gly, Met, D-Ala, Ala, Nle, and Nva;
R¹ is selected from the group consisting of an amino acid side chain of Trp, 2Nal, 1Nal, 4CF₃-Phe, 7Aza-Trp, 1Me-Trp, 5OH-Trp, BIP, 5Ome-Trp, 4F-Phe, 3Pya, 4Pya, PAF, MAF, OAF, 5Qui, 7Ome-Trp, 7Me-Trp, 5F-Trp, 7Cl-Trp, D-Ala, Ala, alpha-Me-Trp, and NMe-Trp;
R² is selected from the group consisting of an amino acid side chain of Thr, D-Ala, Ala, alpha-Me-Thr, Lys, and NMe-Thr;
R³ is selected from the group consisting of an amino acid side chain of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, AIlo-Ile, Lys(C12), Lys(C14), Lys(C16), alpha-Me-Ile, and NMe-tBuAla;
A¹ is selected from the group consisting of:

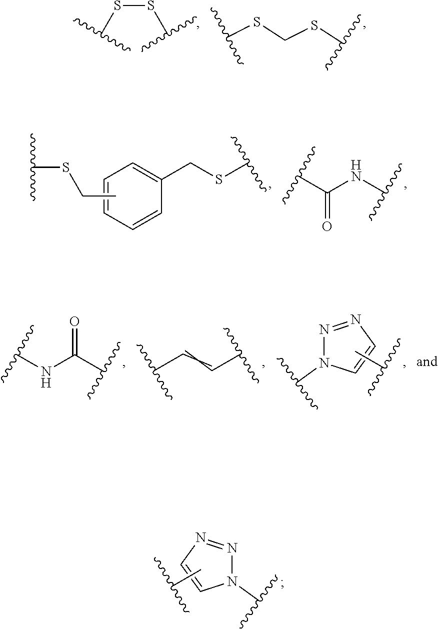

R⁴ is selected from the group consisting of an amino acid side chain of Asn, D-Ala, Ala, DAB-4-NHCOC₅H₁₁, DAB-4-NHCOC₇H₁₅, Asp, Ser, Lys(DOTA), Lys, 3-(4-piperidinyl)-Ala, 3-(1-morpholinyl)-Ala, 3Pya, 4Pya, Glu, NMe-Asn, Pip(CH₂CO₂H)Ala, Pip(PegNMe₃)Ala, and Pip(GAE-DOTA)Ala;
R⁵ is selected from the group consisting of an amino acid side chain of Asn, Ala, D-Ala, Trp, Asp, Lys, Lys(DOTA), 3Pya, 4Pya, 3-(4-piperidinyl)-Ala, 3-(1-morpholinyl)-Ala, Glu, NMe-Asn, and Ser;
R⁶ is selected from the group consisting of an amino acid side chain of Trp, 4CF₃-Phe, 1Me-Trp, 7Aza-Trp, BIP, 2Nal, 1Nal, aMe-Trp, D-Ala, Ala, 4F-Phe, 5F-Trp, 5Ome-Trp, Asn, 5OH-Trp, 7Me-Trp, 7MeO-Trp, 7Cl-Trp, and NMe-Trp;
R⁷ is
(i) selected from the group consisting of an amino acid side chain of 3Pya, 4Pya, Lys(Me)₃, His, Ala, Phe, D-Ala, Gln, Lys, Glu, Arg, Orn, NMe-His, Trp, and Ser; or
(ii) selected from the group consisting of:

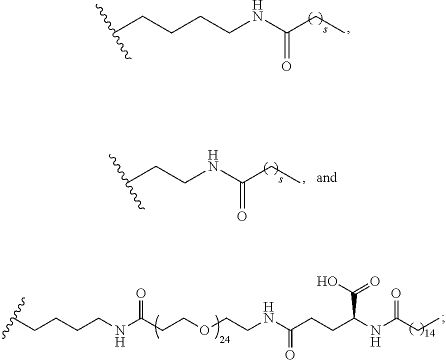

s is 3, 5, 10, 12, or 14;
R⁸ is selected from the group consisting of an amino acid side chain of Asp, D-Ala, Ala, Asn, Thr, NMe-Asp, and alpha-Me-Asp;
R⁹ is selected from the group consisting of an amino acid side chain of Trp, 7Aza-Trp, 1Me-Trp, D-Ala, Ala, 4F-Phe, 1Nal, 2Nal, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, D-Ala, Ala, alpha-Me-Pro, trans4Fluoro-Pro, cis4Fluoro-Pro, trans40H-Pro, cis4OH-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACI, and 3Me2-Aze.

In another embodiment, A¹ is selected from the group consisting of:

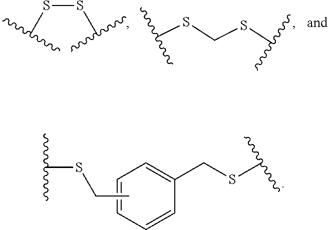

In yet another embodiment, A¹ is:

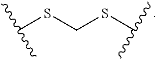 [SMILES: *SCS*]

In still another embodiment, m is 0.

In an embodiment, m is 0 or 1;
P² is

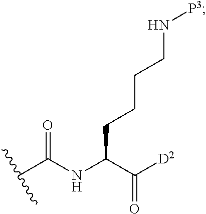

D² is OH or NH₂;
P³ is selected from the group consisting of DOTA,

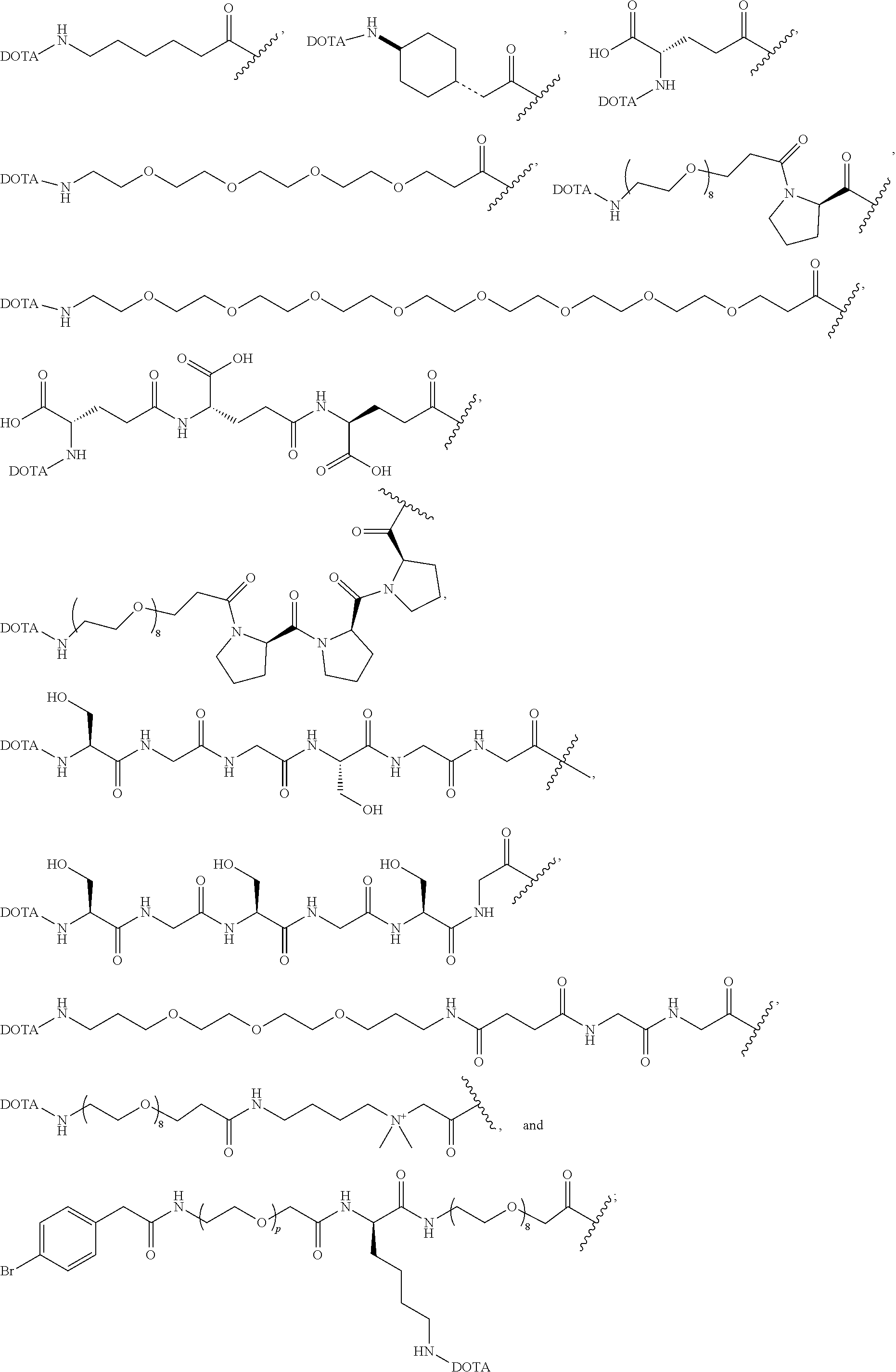

and
p is 8, 12, or 24.

In an embodiment, m is 0 or 1;
P¹ is selected from the group consisting of Ac,

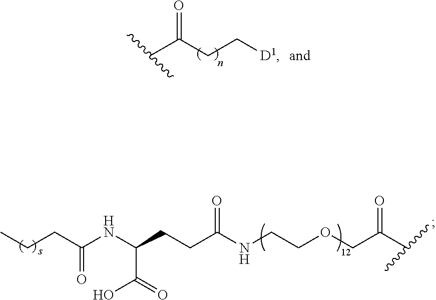

n and s are each independently 9, 10, 11, 12, or 13;
D¹ is CH₃ or C(O)OH;
P² is

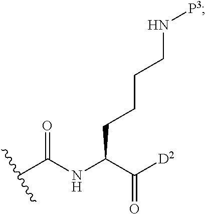

D² is OH or NH₂;
P³ is selected from the group consisting of Chelator,

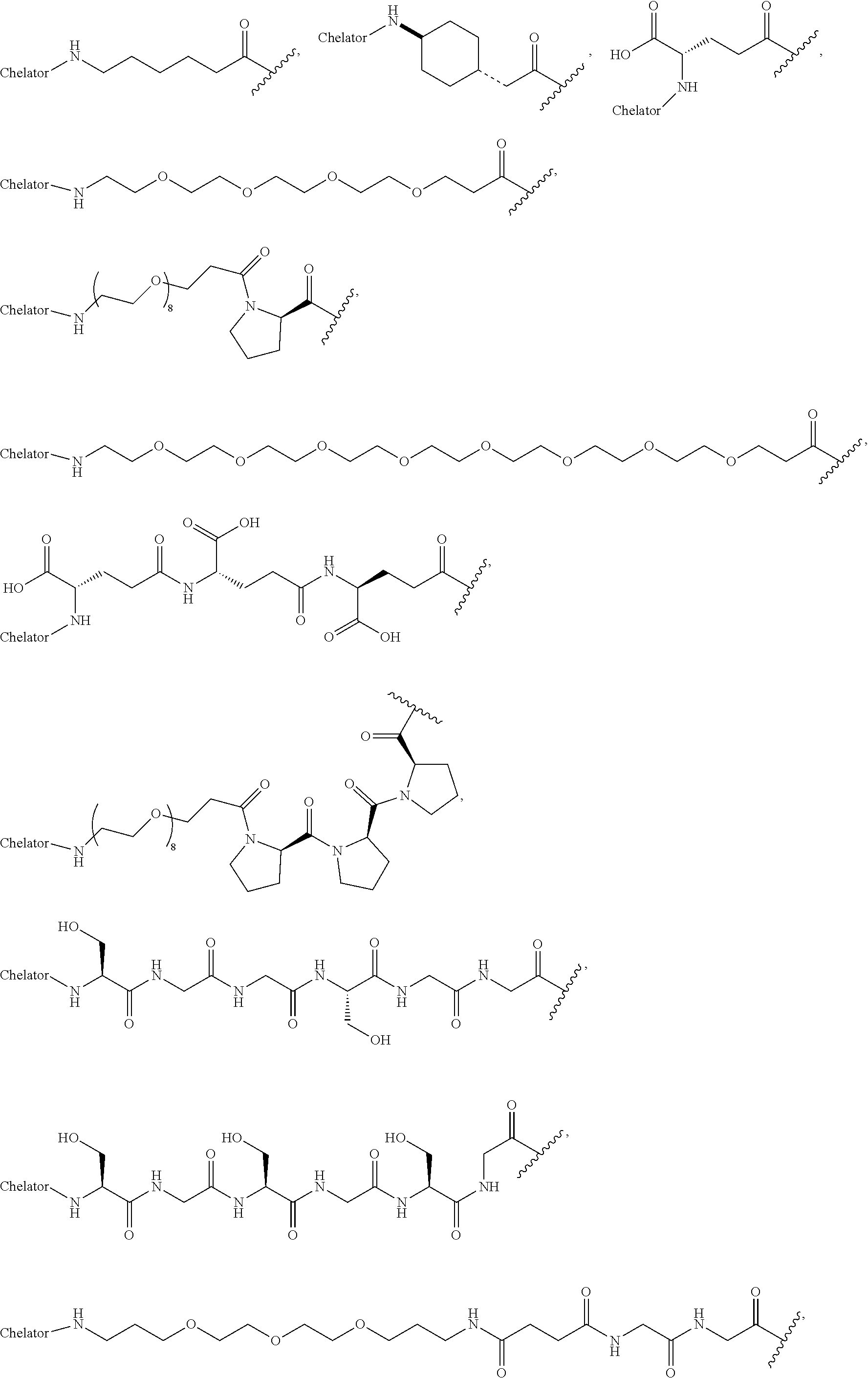

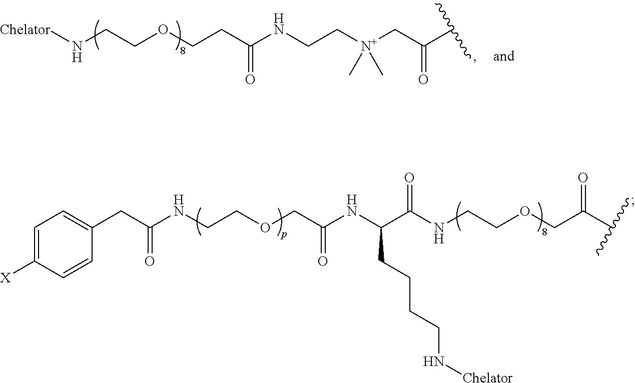

p is an integer selected from 8-24;
R⁰ is selected from the group consisting of an amino acid side chain of Gly, Met, D-Ala, Ala, Nle, and Nva;
R¹ is selected from the group consisting of an amino acid side chain of Trp, 2Nal, 1Nal, 7Aza-Trp, 1Me-Trp, 5OH-Trp, BIP, 5Ome-Trp, 3Pya, 4Pya, 5Qui, 7Ome-Trp, 7Me-Trp, 5F-Trp, 7Cl-Trp, alpha-Me-Trp, and NMe-Trp;
R² is selected from the group consisting of an amino acid side chain of Thr, D-Ala, Ala, alpha-Me-Thr, Lys, and NMe-Thr;
R³ is selected from the group consisting of an amino acid side chain of Ile, Env, Nle, D-Ala, Ala, Leu, t-Bu-Ala, NMe-Nle, α-tert-amylGly, AIlo-Ile, Lys(C12), Lys(C14), Lys(C16), alpha-Me-Ile, and NMe-tBuAla;
A¹ is selected from the group consisting of:

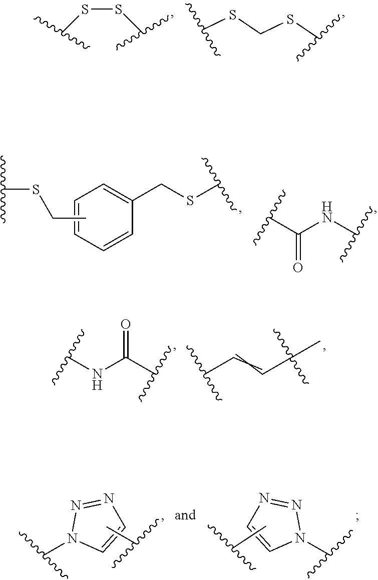

R⁴ is selected from the group consisting of an amino acid side chain of Asn, D-Ala, Ala, DAB-4-NHCOC₅H₁₁, DAB-4-NHCOC₇H₁₅, Asp, Ser, Lys(DOTA), Lys, 3-(4-piperidinyl)-Ala, PipA(acetic), 3-(1-morpholinyl)-Ala, 3Pya, Glu, and NMe-Asn;
R⁵ is selected from the group consisting of an amino acid side chain of Asn, Ala, D-Ala, Asp, Lys, Glu, NMe-Asn, and Ser;
R⁶ is selected from the group consisting of an amino acid side chain of Trp, 4CF₃-Phe, 1Me-Trp, 7Aza-Trp, BIP, 2Nal, 1Nal, aMe-Trp, 4F-Phe, 5F-Trp, 5Ome-Trp, 5OH-Trp, 7Me-Trp, 7MeO-Trp, 7Cl-Trp, and NMe-Trp;
R⁷ is selected from the group consisting of an amino acid side chain of 3Pya, 4Pya, Lys(Me)₃, His, Ala, Phe, D-Ala, Gln, Lys, Glu, Arg, Orn, NMe-His, Trp, and Ser; or
R⁸ is selected from the group consisting of an amino acid side chain of Asp, D-Ala, Ala, Asn, Thr, NMe-Asp, and alpha-Me-Asp;
R⁹ is selected from the group consisting of an amino acid side chain of Trp, 7Aza-Trp, 1Me-Trp, 1Nal, 2Nal, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, alpha-Me-Pro, trans4Fluoro-Pro, cis4Fluoro-Pro, trans40H-Pro, cis4OH-Pro, 5,5-diMe-Pro, trans4NH2-Pro, cis4NH2-Pro, Aze, ACI, and 3Me2-Aze.

In an embodiment,
P¹ is:

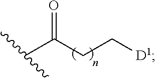

n is an integer selected from 0 to 16;
D¹ is selected from the group consisting of H, CH₃, and C(O)OH;
P² is

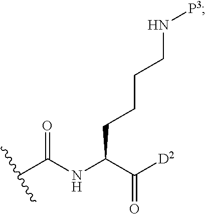

D² is OH or NH₂;
P³ is selected from the group consisting of Chelator,

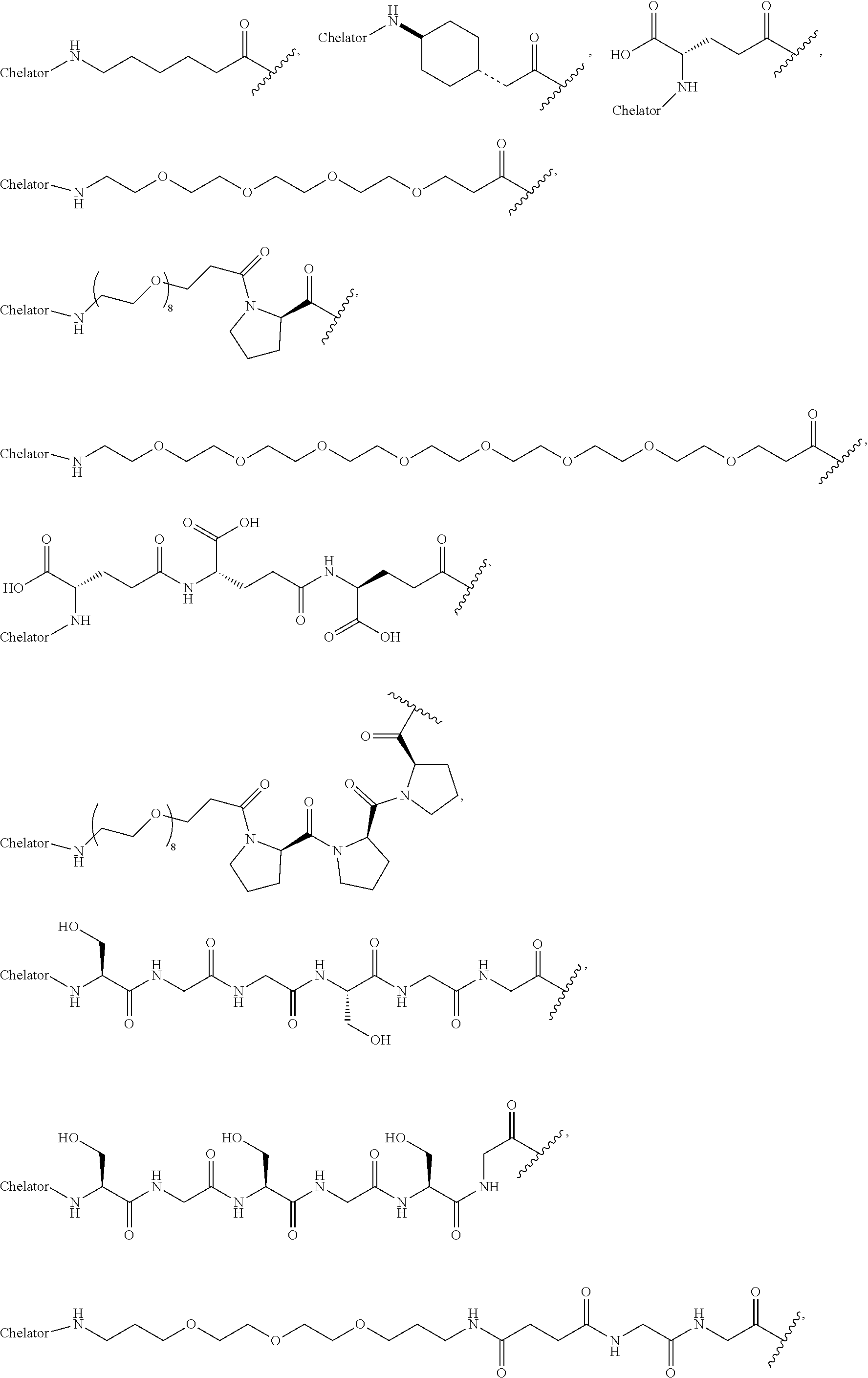

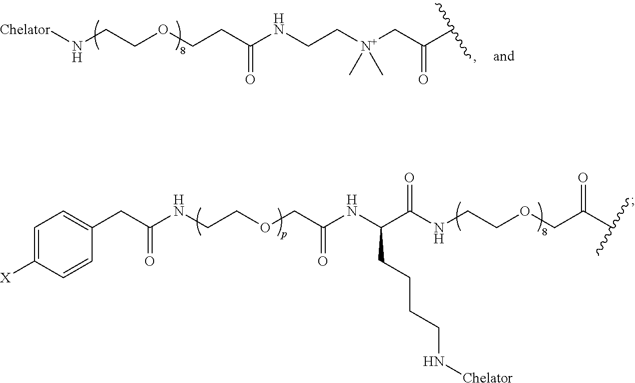

p is 8, 12, or 24;
R⁰ is selected from the group consisting of an amino acid side chain of Gly, Met, 0-Ala, Ala, Nle, and Nva;
R¹ is selected from the group consisting of an amino acid side chain of Trp, 7Aza-Trp, 1Me-Trp, 5OH-Trp, 5Ome-Trp, 7Ome-Trp, 7Me-Trp, 5F-Trp, 7Cl-Trp, alpha-Me-Trp, and NMe-Trp;
R² is selected from the group consisting of an amino acid side chain of Thr, alpha-Me-Thr, Lys, and NMe-Thr;
R³ is selected from the group consisting of an amino acid side chain of D-Ala, Ala, t-Bu-Ala, and NMe-tBuAla;
A¹ is selected from the group consisting of:

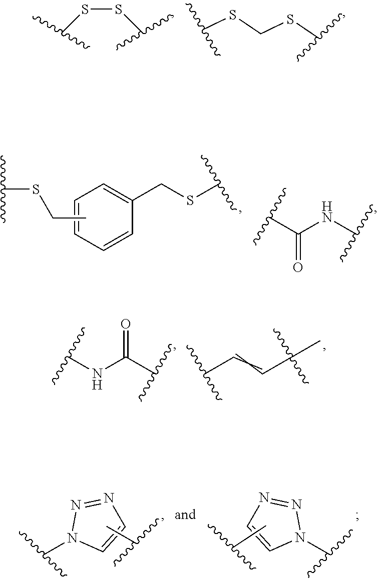

R⁴ is selected from the group consisting of an amino acid side chain of 3-(4-piperidinyl)-Ala, PipA(acetic), and 3-(1-morpholinyl)-Ala;
R⁵ is selected from the group consisting of an amino acid side chain of Asn, Asp, and NMe-Asn;
R⁶ is an amino acid side chain of 2Nal or 1Nal;
R⁷ is selected from the group consisting of an amino acid side chain of 3Pya, 4Pya, Lys(Me)₃, His, Ala, Phe, D-Ala, Gln, Lys, Glu, Arg, Orn, NMe-His, Trp, and Ser;
R⁸ is selected from the group consisting of an amino acid side chain of Asp, Asn, NMe-Asp, and alpha-Me-Asp;
R⁹ is selected from the group consisting of an amino acid side chain of Trp, 7Aza-Trp, 1Me-Trp, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, alpha-Me-Pro, trans4Fluoro-Pro, cis4Fluoro-Pro, trans40H-Pro, cis4OH-Pro, 5,5-diMe-Pro, trans4NH2-Pro, and cis4NH2-Pro.

In another embodiment,
R¹ is an amino side chain of Trp;
R² is an amino side chain of Thr;
R³ is an amino side chain of Ile or NMe-tBuAla;
R⁴ is selected from an amino side chain of Lys, 3-(4-piperidinyl)-Ala, and PipA(acetic);
R⁵ is an amino side chain of Asn or NMe-Asn;
R⁶ is an amino side chain of Trp or 2Nal;
R⁷ is selected from an amino side chain of His or Lys;
R⁸ is an amino side chain of Asp;
R⁹ is an amino side chain of Trp; and
R¹⁰ is an amino side chain of Pro.

In another embodiment,
R¹ is an amino side chain of Trp;
R² is an amino side chain of Thr;
R³ is an amino side chain of Ile;
R⁴ is selected from an amino side chain of Lys, 3-(4-piperidinyl)-Ala, and PipA(acetic);
R⁵ is an amino side chain of Asn or NMe-Asn;
R⁶ is an amino side chain of 2Nal or Trp;
R⁷ is selected from an amino side chain of His;
R⁸ is an amino side chain of Asp;
R⁹ is an amino side chain of Trp; and
R¹⁰ is an amino side chain of Pro.

In another embodiment,
R¹ is an amino acid side chain of Trp;
R² is an amino acid side chain of Thr;
R³ is an amino acid side chain of NMe-tBuAla;
R⁴ is an amino acid side chain of 3-(4-piperidinyl)-Ala or PipA(acetic);
R⁵ is an amino acid side chain of Asn;
R⁶ is an amino acid side chain of 2Nal;
R⁷ is an amino acid side chain of Lys
R⁸ is an amino acid side chain of Asp;
R⁹ is an amino acid side chain of Trp; and
R¹⁰ is an amino acid side chain of Pro.

In an embodiment,
P¹ is:

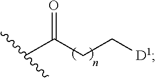

n is an integer from 0 to 8;
P² is

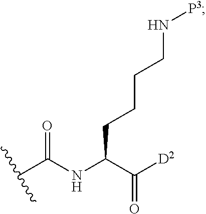

D² is OH or NH₂;
P³ is selected from the group consisting of Chelator,

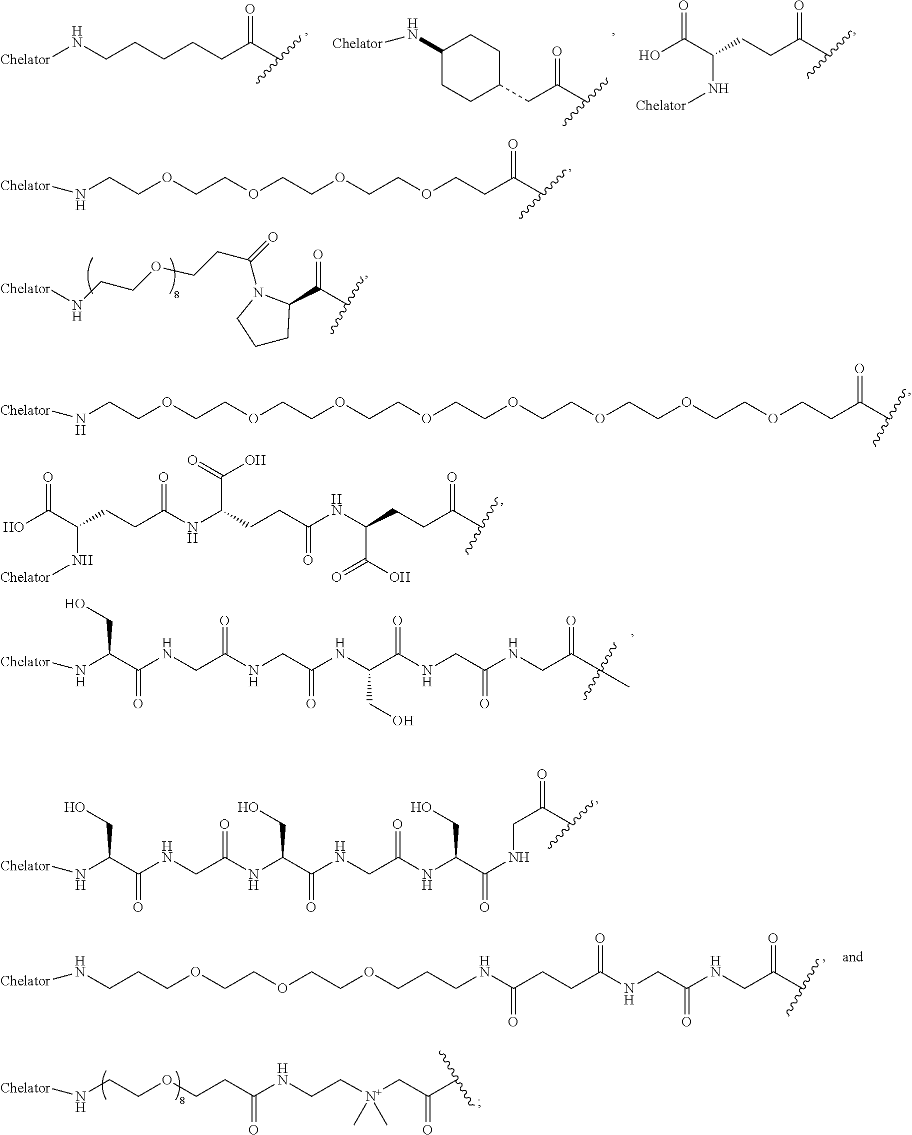

R¹ is selected from the group consisting of an amino acid side chain of Trp, 7Aza-Trp, 1Me-Trp, 5OH-Trp, 5Ome-Trp, 7Ome-Trp, 7Me-Trp, 5F-Trp, 7Cl-Trp, alpha-Me-Trp, and NMe-Trp;
R² is an amino acid side chain of Thr;
R³ is selected from the group consisting of an amino acid side chain of D-Ala, Ala, t-Bu-Ala, and NMe-tBuAla;
A¹ is

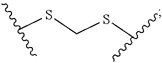 [SMILES: *SCS*]

B¹ is CH₂;
C¹ is CH₂;
R⁴ is selected from the group consisting of an amino acid side chain of 3-(4-piperidinyl)-Ala, PipA(acetic), and 3-(1-morpholinyl)-Ala;
R⁵ is selected from the group consisting of an amino acid side chain of Asn, Asp, and NMe-Asn;
R⁶ is an amino acid side chain of 2Nal or 1Nal;
R⁷ is selected from the group consisting of an amino acid side chain of His, Lys, and Trp;
R⁸ is selected from the group consisting of an amino acid side chain of Asp, NMe-Asp, and alpha-Me-Asp;
R⁹ is selected from the group consisting of an amino acid side chain of Trp, 7Aza-Trp, 1Me-Trp, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, alpha-Me-Pro, trans4Fluoro-Pro, cis4Fluoro-Pro, trans40H-Pro, cis4OH-Pro, 5,5-diMe-Pro, trans4NH2-Pro, and cis4NH2-Pro.

In an embodiment, P¹ is:

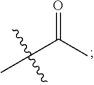 [SMILES: *C(C)=O]

P² is

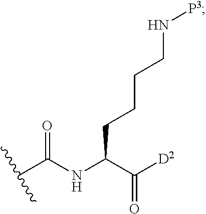

P³ is selected from the group consisting of Chelator,

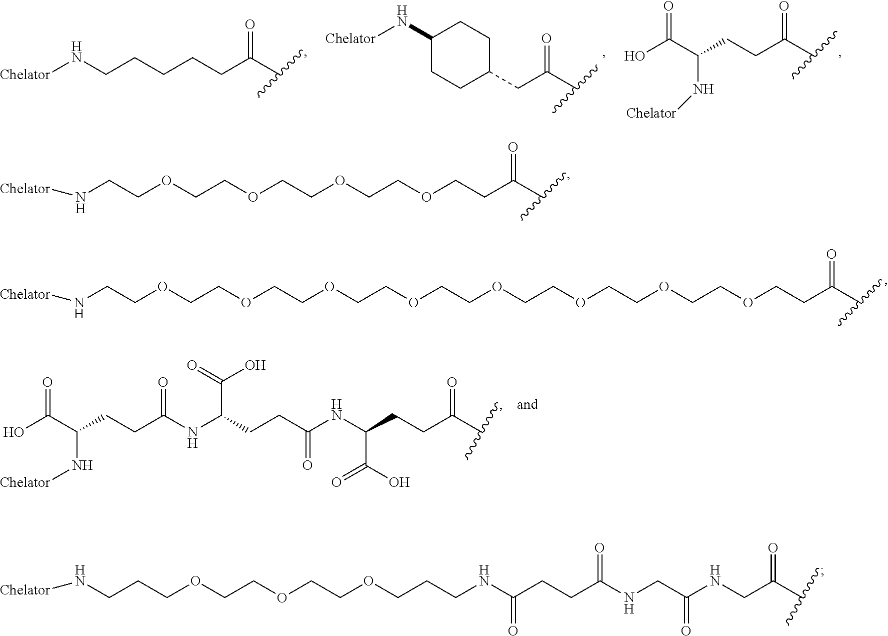

R¹ is selected from the group consisting of an amino acid side chain of Trp, 7Aza-Trp, 1Me-Trp, 5OH-Trp, 5Ome-Trp, 7Ome-Trp, 7Me-Trp, 5F-Trp, 7Cl-Trp, alpha-Me-Trp, and NMe-Trp;
R² is selected from the group consisting of an amino acid side chain of Thr, alpha-Me-Thr, and NMe-Thr;
R³ is selected from the group consisting of an amino acid side chain of D-Ala, Ala, t-Bu-Ala, and NMe-tBuAla;
A¹ is

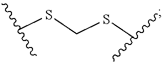 [SMILES: *SCS*]

B¹ is CH₂;
C¹ is CH₂;
R⁴ is selected from the group consisting of an amino acid side chain of 3-(4-piperidinyl)-Ala, PipA(acetic), and 3-(1-morpholinyl)-Ala;
R⁵ is an amino acid side chain of Asn or NMe-Asn;
R⁶ is an amino acid side chain of 2Nal or 1Nal;
R⁷ is selected from the group consisting of an amino acid side chain of His, Lys, and Trp;
R⁸ is selected from the group consisting of an amino acid side chain of Asp, NMe-Asp, and alpha-Me-Asp;
R⁹ is selected from the group consisting of an amino acid side chain of Trp, 7Aza-Trp, 1Me-Trp, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, alpha-Me-Pro, trans4Fluoro-Pro, cis4Fluoro-Pro, trans40H-Pro, cis4OH-Pro, 5,5-diMe-Pro, trans4NH2-Pro, and cis4NH2-Pro.

In an embodiment,
P¹ is:

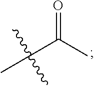 [SMILES: *C(C)=O]

P² is

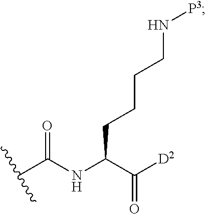

P³ is selected from the group consisting of Chelator,

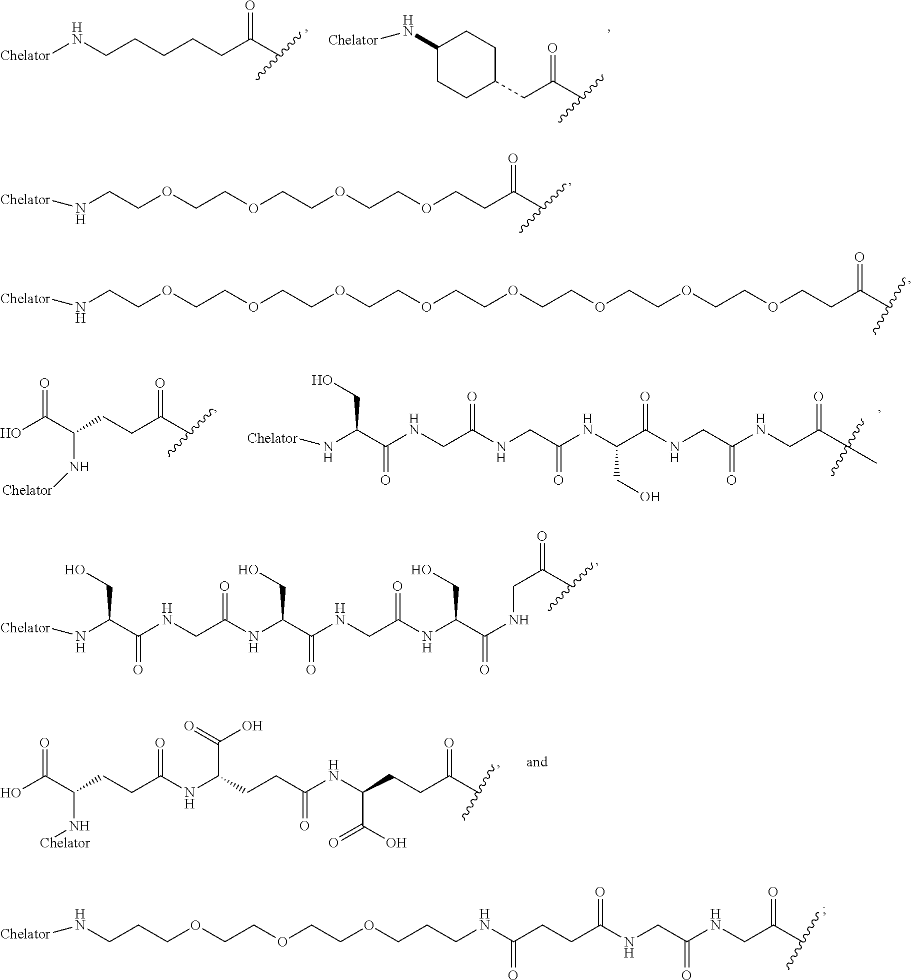

R¹ is an amino acid side chain of Trp;
R² is an amino acid side chain of Thr;
R³ is an amino acid side chain of Ile or NMe-tBuAla;
A¹ is

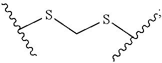 [SMILES: *SCS*]

B¹ is CH₂;
C¹ is CH₂;
R⁴ is an amino acid side chain of Lys, 3-(4-piperidinyl)-Ala or PipA(acetic);
R⁵ is an amino acid side chain of Asn or NMe-Asn;
R⁶ is an amino acid side chain of Trp or 2Nal;
R⁷ is an amino acid side chain of His or Lys;
R⁸ is an amino acid side chain of Asp;
R⁹ is an amino acid side chain of Trp; and
R¹⁰ is an amino acid side chain of Pro.

In an embodiment,
P¹ is:

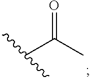 [SMILES: *C(C)=O]

P² is

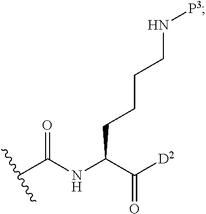

P³ is selected from the group consisting of Chelator,

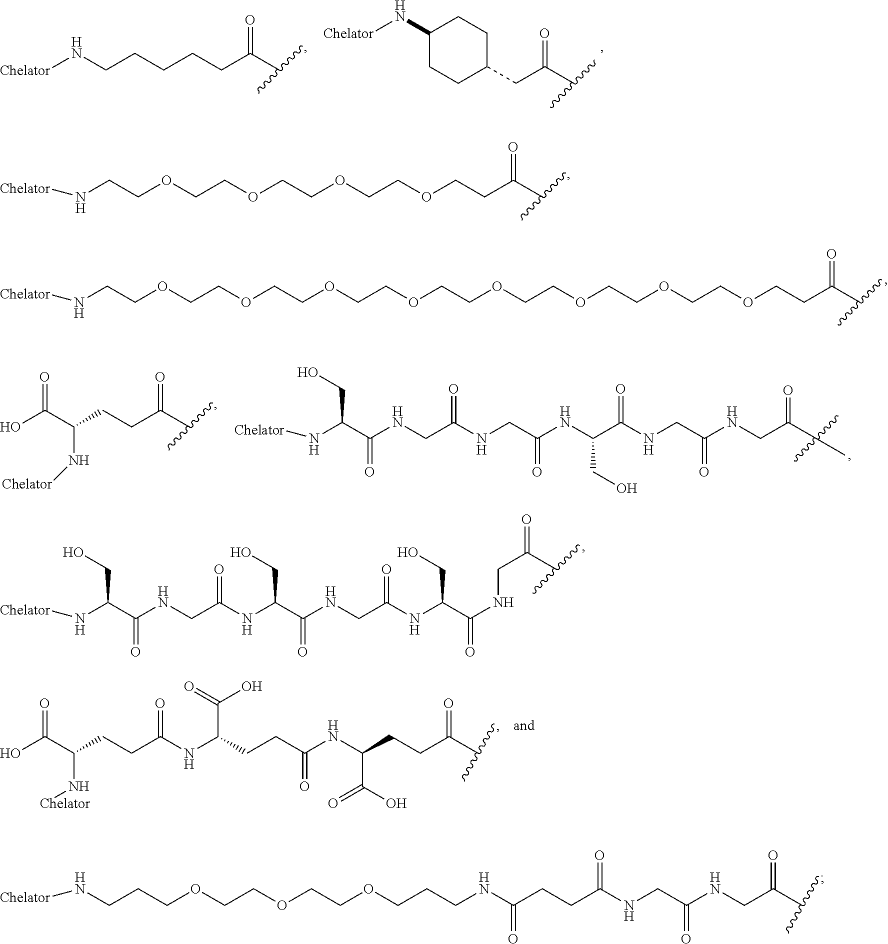

R¹ is an amino acid side chain of Trp;
R² is an amino acid side chain of Thr;
R³ is an amino acid side chain of Ile;
A¹ is

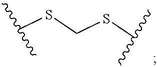 [SMILES: *SCS*]

B¹ is CH₂;
C¹ is CH₂;
R⁴ is an amino acid side chain of 3-(4-piperidinyl)-Ala or PipA(acetic);
R⁵ is an amino acid side chain of NMe-Asn;
R⁶ is an amino acid side chain of 2Nal;
R⁷ is an amino acid side chain of His;
R⁸ is an amino acid side chain of Asp;
R⁹ is an amino acid side chain of Trp; and
R¹⁰ is an amino acid side chain of Pro.

In an embodiment,
P¹ is:

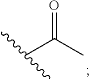 [SMILES: *C(C)=O]

P² is

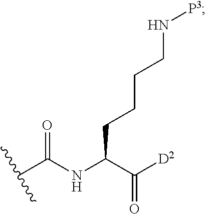

P³ is selected from the group consisting of DOTA,

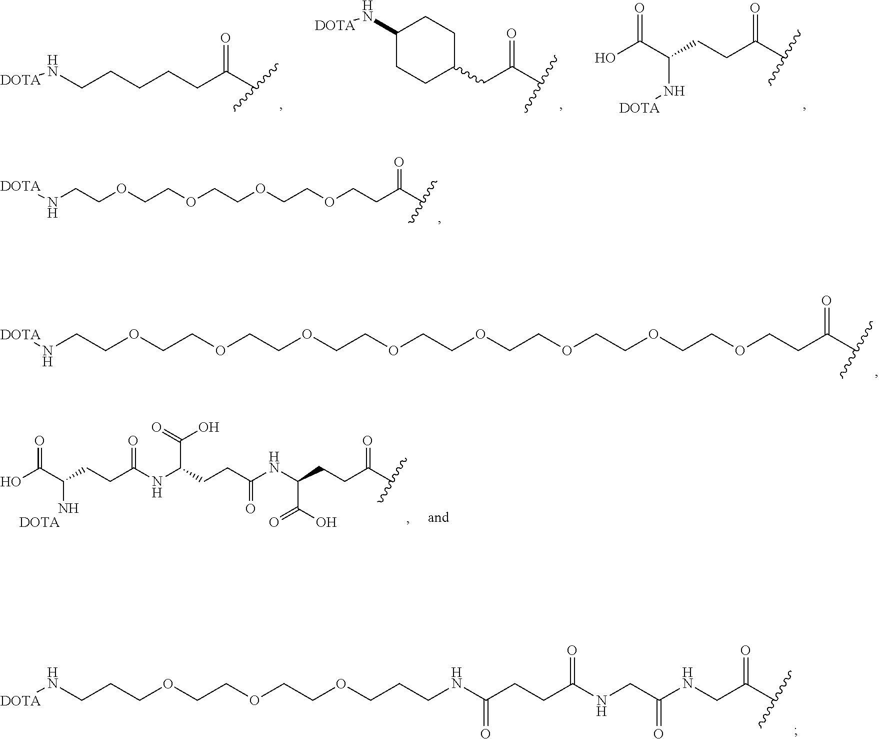

R¹ is an amino acid side chain of Trp;
R² is an amino acid side chain of Thr;
R³ is an amino acid side chain of NMe-tBuAla;
A¹ is

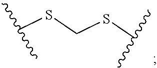 [SMILES: *SCS*]

B¹ is CH₂;
C¹ is CH₂;
R⁴ is an amino acid side chain of 3-(4-piperidinyl)-Ala or PipA(acetic);
R⁵ is an amino acid side chain of Asn;
R⁶ is an amino acid side chain of 2Nal;
R⁷ is an amino acid side chain of Lys;
R^{a} is an amino acid side chain of Asp;
R⁹ is an amino acid side chain of Trp; and
R¹⁰ is an amino acid side chain of Pro.

In another embodiment, R⁰ is selected from the group consisting of an amino acid side chain of Gly, Met, D-Ala, Ala, Nle, and Nva.

In yet another embodiment, R¹ is selected from the group consisting of an amino acid side chain of Trp, 2Nal, 1Nal, 4CF₃-Phe, 7Aza-Trp, 1Me-Trp, 5OH-Trp, BIP, 5Ome-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp. In still another embodiment, R¹ is selected from the group consisting of an amino acid side chain of Trp, 7Aza-Trp, 1Me-Trp, 5OH-Trp, 5Ome-Trp, 7Ome-Trp, 7Me-Trp, 5F-Trp, 7Cl-Trp, alpha-Me-Trp, and NMe-Trp. In an embodiment, R¹ is an amino side chain of Trp or 7Me-Trp. In another embodiment, R¹ is an amino side chain of Trp.

In yet another embodiment, R² is selected from the group consisting of an amino acid side chain of Thr, D-Ala, Ala, alpha-Me-Thr, Lys, and NMe-Thr. In still another embodiment, R² is selected from the group consisting of an amino acid side chain of Thr, alpha-Me-Thr, and NMe-Thr. In an embodiment, R² is an amino side chain of Thr.

In an embodiment, R³ is selected from the group consisting of an amino acid side chain of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, AIlo-Ile, Lys(C12), Lys(C14), Lys(C16), alpha-Me-Ile, and NMe-tBuAla. In another embodiment, R³ is selected from the group consisting of an amino acid side chain of Ile, AIlo-Ile, alpha-Me-Ile, and NMe-tBuAla. In yet another embodiment, R³ is selected from the group consisting of an amino acid side chain of D-Ala, Ala, t-Bu-Ala, and NMe-tBuAla. In still another embodiment, R³ is an amino side chain of Ile or NMe-tBuAla. In an embodiment, R³ is an amino side chain of Ile. In another embodiment, R³ is an amino side chain of NMe-tBuAla.

In yet another embodiment, R⁴ is selected from the group consisting of an amino acid side chain of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, NMe-Asn, Pip(CH₂CO₂H)Ala, and Pip(PegNMe₃)Ala. In still another embodiment, R⁴ is selected from the group consisting of an amino acid side chain of 3-(4-piperidinyl)-Ala, PipA(acetic), and 3-(1-morpholinyl)-Ala. In another embodiment, R⁴ is an amino side chain of 3-(4-piperidinyl)-Ala or PipA(acetic).

In yet another embodiment, R⁵ is selected from the group consisting of an amino acid side chain of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser. In still another embodiment, R⁵ is selected from the group consisting of an amino acid side chain of Asn and NMe-Asn. In an embodiment, R⁵ is an amino side chain of NMe-Asn. In another embodiment, R⁵ is an amino side chain of Asn.

In yet another embodiment, R⁶ is selected from the group consisting of an amino acid side chain of Trp, 4CF₃-Phe, 1Me-Trp, 7Aza-Trp, BIP, 2Nal, 1Nal, alpha-Me-Trp, D-Ala, Ala, 4F-Phe, 5F-Trp, 5MeO-Trp, Asn, 5OH-Trp, 7Me-Trp, 7MeO-Trp, 7Cl-Trp, and NMe-Trp. In still another embodiment, R⁶ is selected from the group consisting of an amino acid side chain of Trp, 1Me-Trp, 7Aza-Trp, 2Nal, 1Nal, alpha-Me-Trp, 5F-Trp, 5MeO-Trp, 5OH-Trp, 7Me-Trp, 7MeO-Trp, 7Cl-Trp, and NMe-Trp. In an embodiment, R⁶ is selected from the group consisting of an amino acid side chain of Trp, 2Nal and 1Nal. In another embodiment, R⁶ is an amino acid side chain of Trp. In yet another embodiment, R⁶ is an amino side chain of 2Nal.

In still another embodiment, R⁷ is selected from the group consisting of an amino acid side chain of 3Pya, 4Pya, Lys(Me)3, His, Ala, D-Ala, Gln, Lys, Glu, Arg, Orn, NMe-His, and Ser. In an embodiment, R⁷ is an amino acid side chain of His or Lys. In an embodiment, R⁷ is an amino acid side chain of His. In an embodiment, R⁷ is an amino acid side chain of Lys.

In still another embodiment, R⁸ is selected from the group consisting of an amino acid side chain of Asp, D-Ala, Ala, Asn, Thr, NMe-Asp, and alpha-Me-Asp. In an embodiment, R⁸ is selected from the group consisting of an amino acid side chain of Asp, Asn, NMe-Asp, and alpha-Me-Asp. In another embodiment, R⁸ is selected from the group consisting of an amino acid side chain of Asp, NMe-Asp, and alpha-Me-Asp. In yet another embodiment, R⁸ is an amino side chain of Asp.

In an embodiment, R⁹ is selected from the group consisting of an amino acid side chain of Trp, 7Aza-Trp, 1Me-Trp, D-Ala, Ala, 4F-Phe, 1Nal, 2Nal, 5F-Trp, 5MeO-Trp, alpha-Me-Trp, 7Cl-Trp, 5OH-Trp, 7Me-Trp, 7MeO-Trp, and NMe-Trp. In an embodiment, R⁹ is selected from the group consisting of an amino acid side chain of Trp, 7Aza-Trp, 1Me-Trp, 5F-Trp, 5Ome-Trp, alpha-Me-Trp, 7Cl-Trp, 5OH-Trp, 7Me-Trp, 7MeO-Trp, and NMe-Trp. In an embodiment, R⁹ is an amino side chain of Trp.

In an embodiment, R¹⁰ is selected from the group consisting of an amino acid side chain of Pro, D-Ala, Ala, alpha-Me-Pro, trans4Fluoro-Pro, cis4Fluoro-Pro, trans40H-Pro, cis4OH-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACI, and 3Me2-Aze. In an embodiment, R¹⁰ is selected from the group consisting of an amino acid side chain of R¹⁰ is selected from the group consisting of an amino acid side chain of Pro, alpha-Me-Pro, trans4Fluoro-Pro, cis4Fluoro-Pro, trans40H-Pro, cis4OH-Pro, 5,5-diMe-Pro, trans4NH2-Pro, and cis4NH2-Pro.

In an embodiment, R¹⁰ is an amino side chain of Pro.

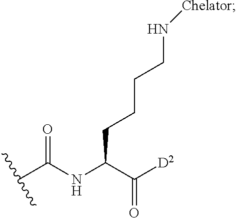

and
D² is OH or NH₂.

In another embodiment, P² is

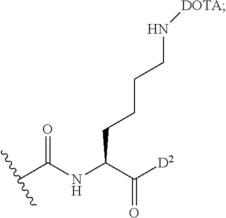

and
D² is OH or NH₂.

In yet another embodiment,
B¹ is CH₂ or C(CH₃)₂; and
C¹ is CH₂ or C(CH₃)₂.

In still another embodiment,
B¹ is CH₂; and
C¹ is CH₂.

In yet another embodiment, Chelator is selected from a Chelator in Table C.

In an embodiment, Chelator is independently selected from a group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N,N′,N″,N′″,N″″,N′″″-hexaacetic acid (HEHA). In another embodiment, Chelator is selected from Deferoxamine (DFO), 5,11,16,22-Tetraazahexacosanediamide (DFO*), and N,N′-1,4-Butanediylbis[N-[3-[[(1,6-dihydro-1-hydroxy-6-oxo-2-pyridinyl)carbonyl]amino]propyl]-1,6-dihydro-1-hydroxy-6-oxo-2-pyridinecarboxamide] (HOPO).

In another embodiment, Chelator is DOTA. In yet another embodiment, Chelator is DOTAGA. In still another embodiment, Chelator is Macrodipa. In an embodiment, Chelator is macropa.

In another embodiment, Formula B is substituted by at least one chelator. In yet another embodiment, Formula B is substituted by one chelator. In still another embodiment, Formula B is substituted by at two chelators.

In the formulae provided herein, a variable, e.g., an R⁰, R¹, R², R³, R⁴, R⁵, R⁶, R⁷, R⁸, R⁹, or R¹⁰ group, can be defined as the side chain of a cyclic amino acid, e.g., proline. In that instance, the corresponding amino acid nitrogen of the peptide backbone of the generic formulae provided herein forms part of the cyclic group. For example, “R¹⁰ is selected from the group consisting of an amino acid side chain of Pro, alpha-Me-Pro, trans4Fluoro-Pro, cis4Fluoro-Pro,” etc., is defined as follows:

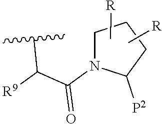

wherein, in this depiction, each R is independently hydrogen or a substituent.

In another embodiment, the compound of Formula B is selected from the group consisting of a compound from Table A.

TABLE A

| | | SEQ |
|---|---|---|
| | Cpd | ID |
| Structure | Nos.# | Nos. |
| 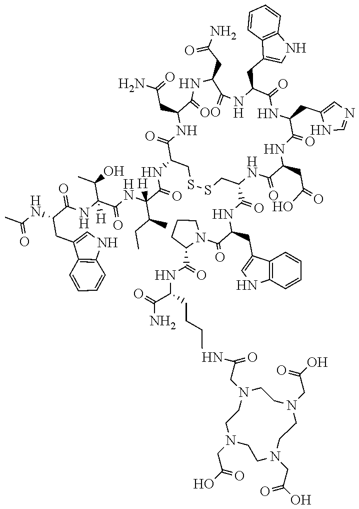 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 23 | 91 |
| C₉₆H₁₃₀N₂₆O₂₆S₂ | | |
| 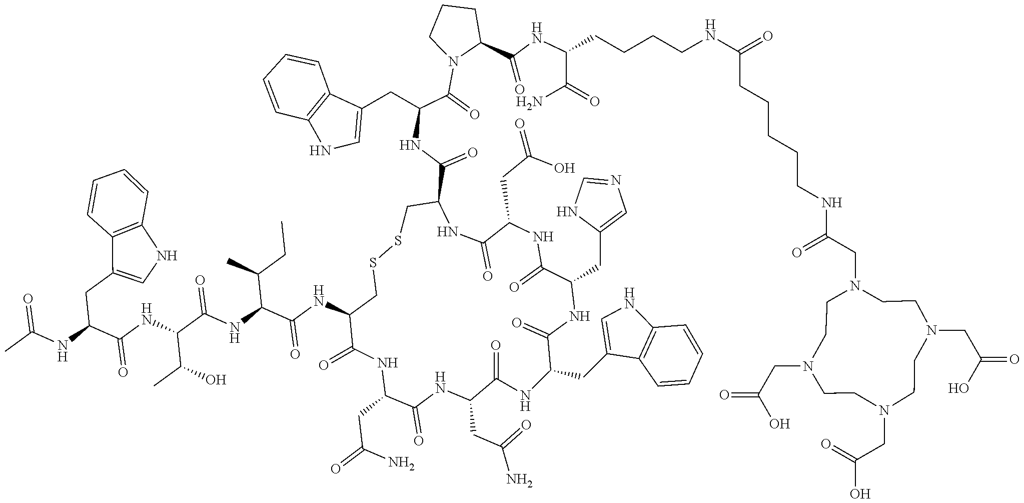 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CCCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 47 | 92 |
| C₁₀₂H₁₄₁N₂₇O₂₇S₂ | | |
| 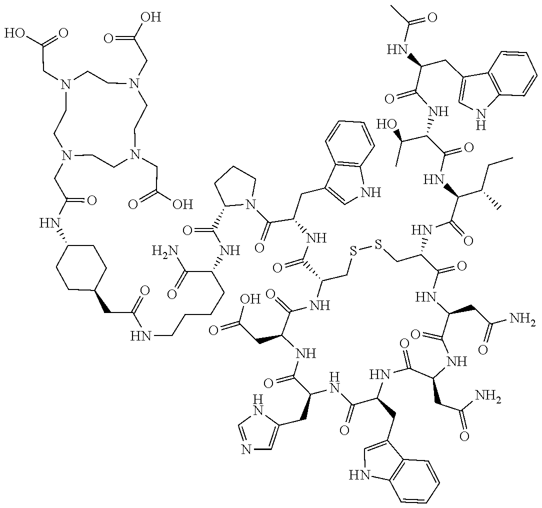 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)C[C@H]2CC[C@H](NC(=O)CN3CCN(CC(=O)O)CCN(CC(=O)O)CCN(CC(=O)O)CC3)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 48 | 93 |
| C₁₀₄H₁₄₃N₂₇O₂₇S₂ | | |
| 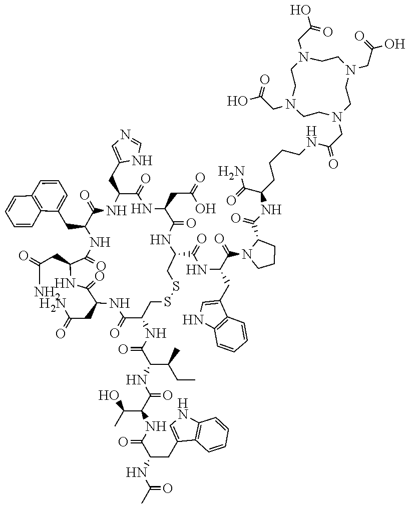 | 74 | 94 |
| C₉₈H₁₃₁N₂₅O₂₆S₂ | | |
| 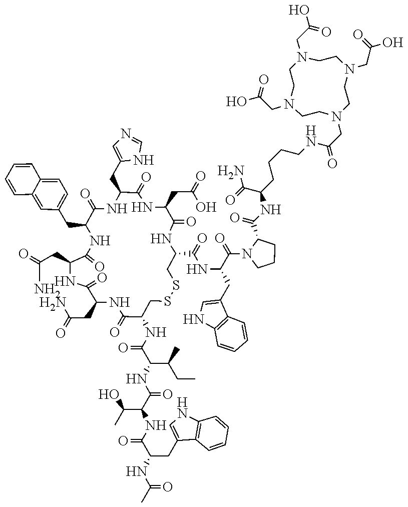 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 75 | 95 |
| C₉₈H₁₃₁N₂₅O₂₆ | | |
| 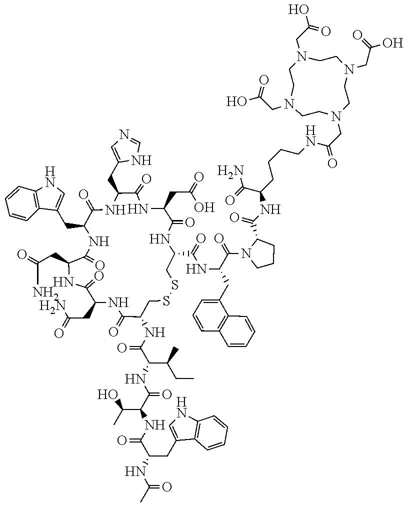 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2cccc3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 76 | 96 |
| C₉₈H₁₃₁N₂₅O₂₆S₂ | | |
| 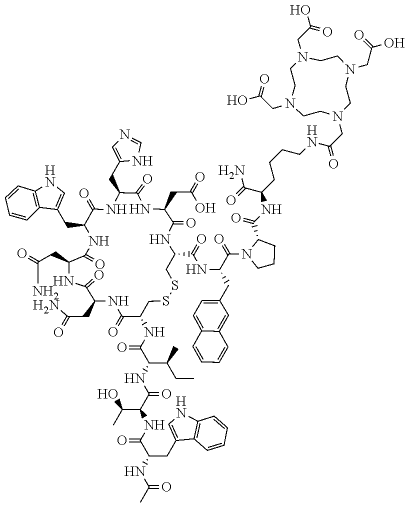 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2ccc3ccccc3c2)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 77 | 97 |
| C₉₈H₁₃₁N₂₅O₂₆S₂ | | |
| 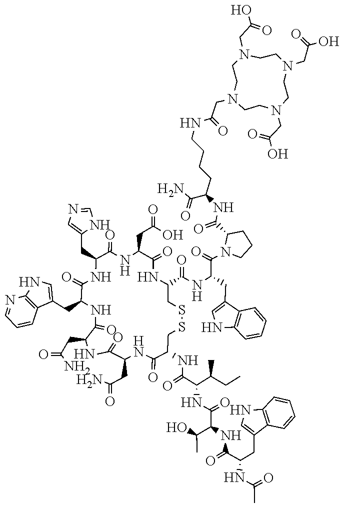 | 85 | 98 |
| C₉₅H₁₂₉N₂₇O₂₆S₂ | | |
| 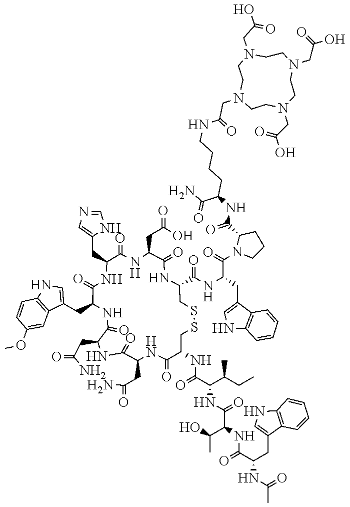 | 86 | 99 |
| C₉₇H₁₃₂N₂₆O₂₇S₂ | | |
| 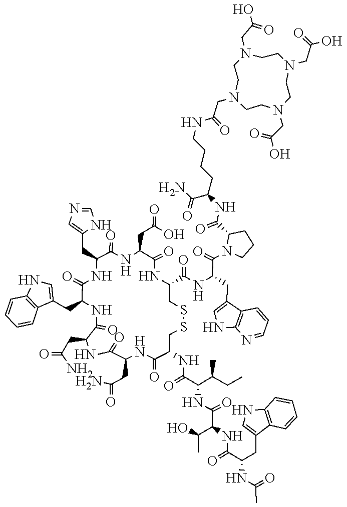 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 87 | 100 |
| C₉₅H₁₂₉N₂₇O₂₆S₂ | | |
| 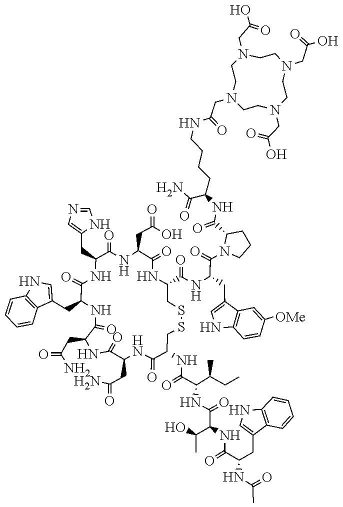 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccc(OC)cc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 88 | 101 |
| C₉₇H₁₃₂N₂₆O₂₇S₂ | | |
| 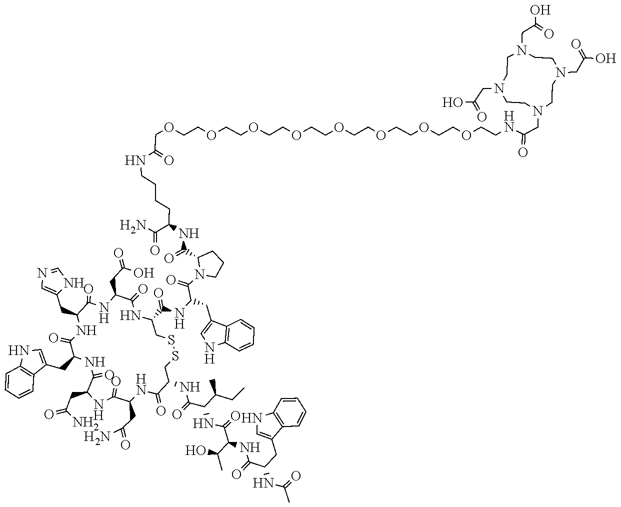 | 89 | 102 |
| C₁₁₄H₁₆₅N₂₇O₃₅S₂ | | |
| 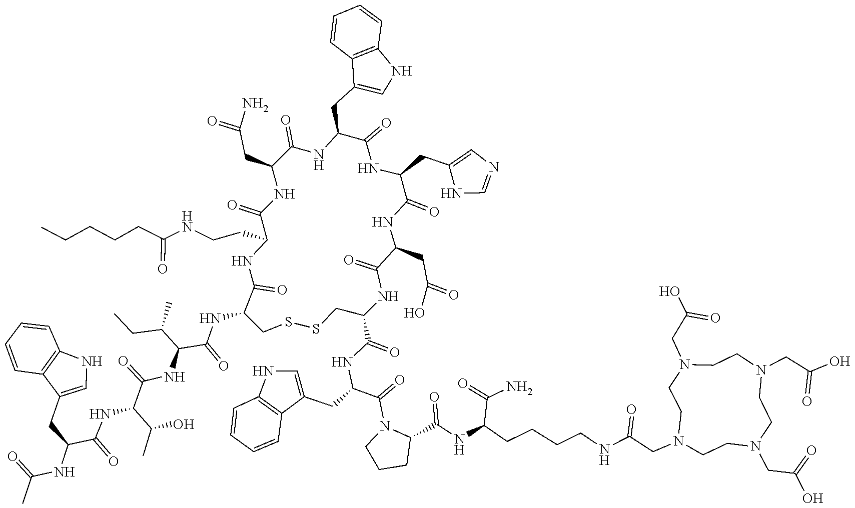 [SMILES: CCCCCC(=O)NCC[C@@H]1NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC1=O] | 90 | 103 |
| C₁₀₂H₁₄₂N₂₆O₂₆S₂ | | |
| 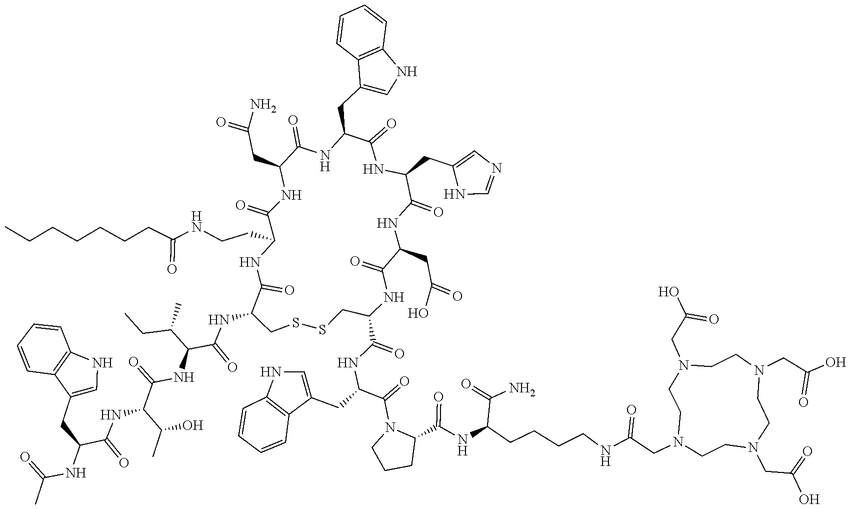 [SMILES: CCCCCCCC(=O)NCC[C@@H]1NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC1=O] | 91 | 104 |
| C₁₀₄H₁₄₆N₂₆O₂₆S₂ | | |
| 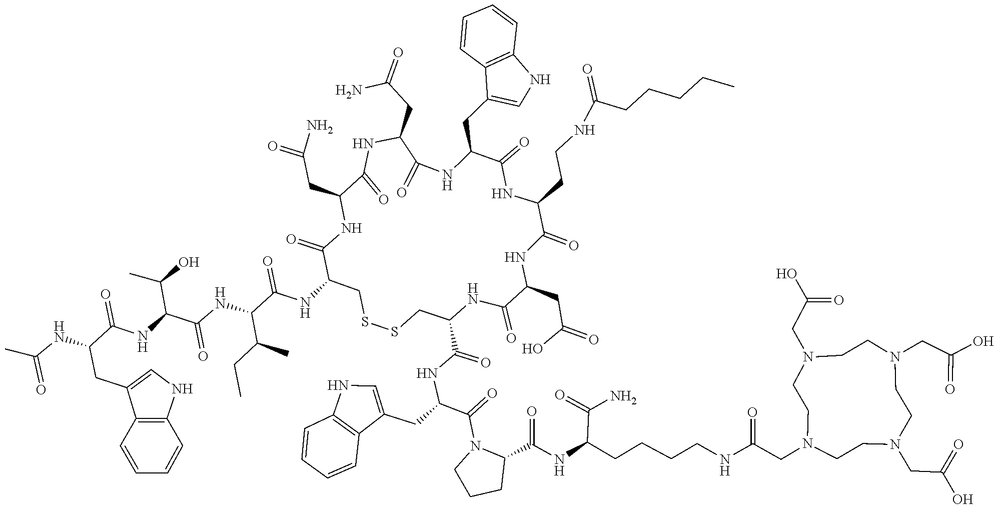 [SMILES: CCCCCC(=O)NCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O] | 92 | 105 |
| C₁₀₀H₁₄₁N₂₅O₂₇S₂ | | |
| 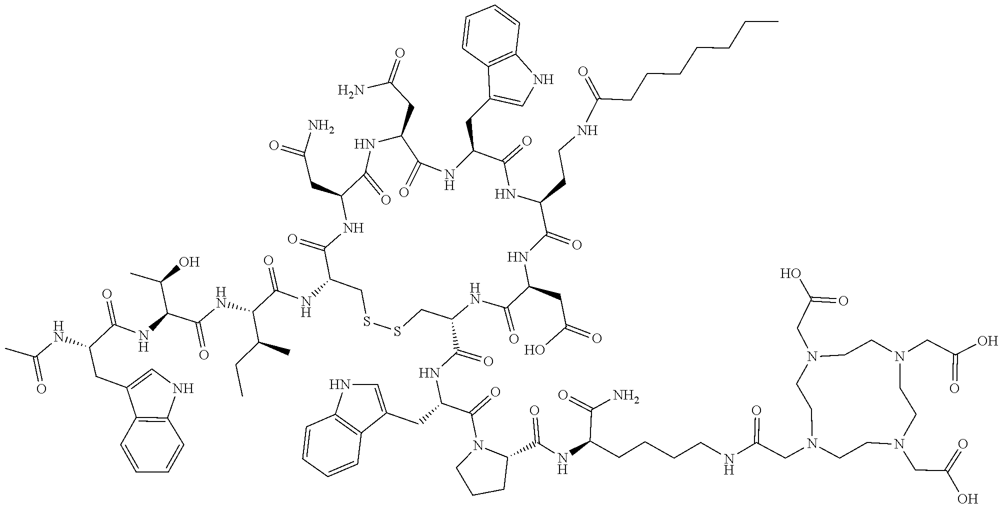 [SMILES: CCCCCCCC(=O)NCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O] | 93 | 106 |
| C₁₀₂H₁₄₅N₂₅O₂₇S₂ | | |
| 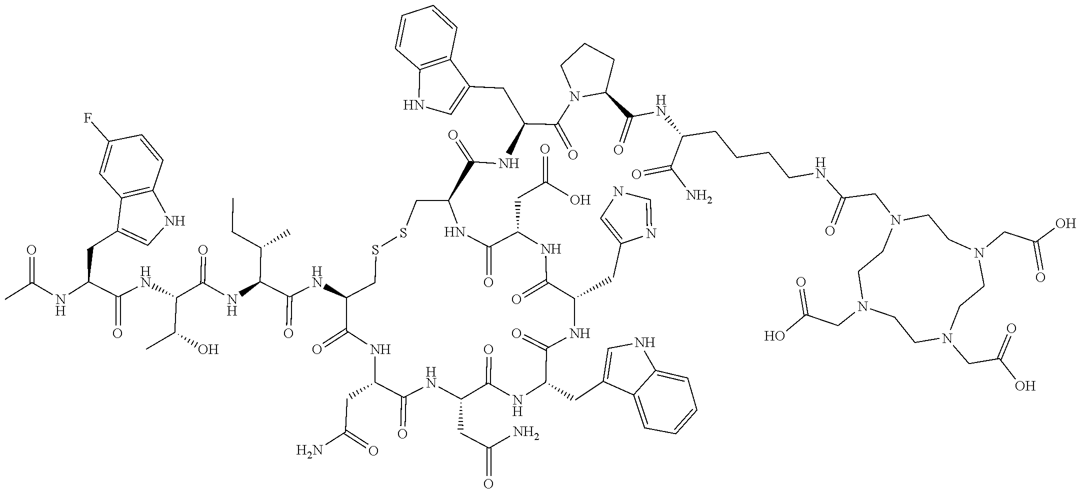 | 100 | 107 |
| C₉₆H₁₂₉FN₂₆O₂₆S₂ | | |
| 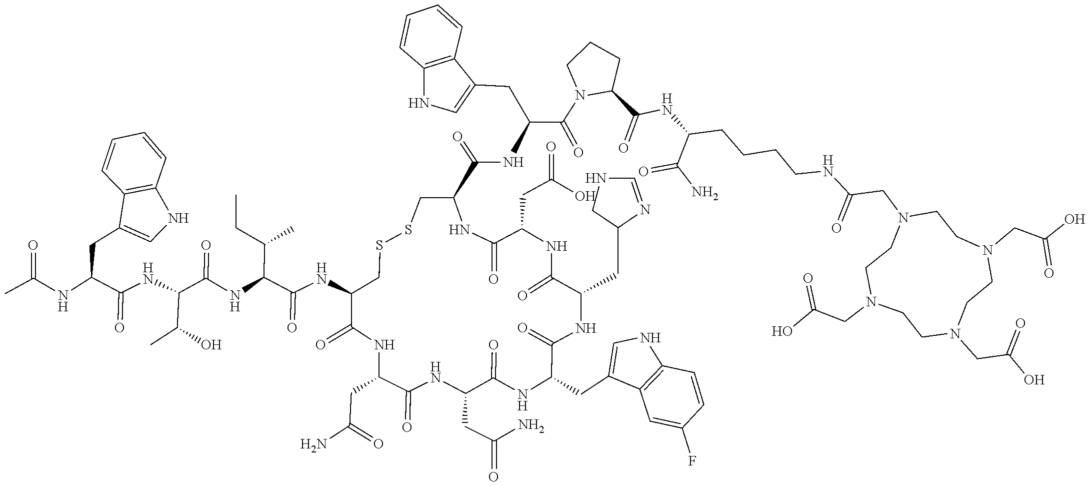 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CC2CNC=N2)NC(=O)[C@H](Cc2c[nH]c3ccc(F)cc23)NC(=O)[C@H](CC(N)=O)NC(=O)[C@H](CC(N)=O)NC1=O] | 101 | 108 |
| C₉₆H₁₂₉FN₂₆O₂₆S₂ | | |
| 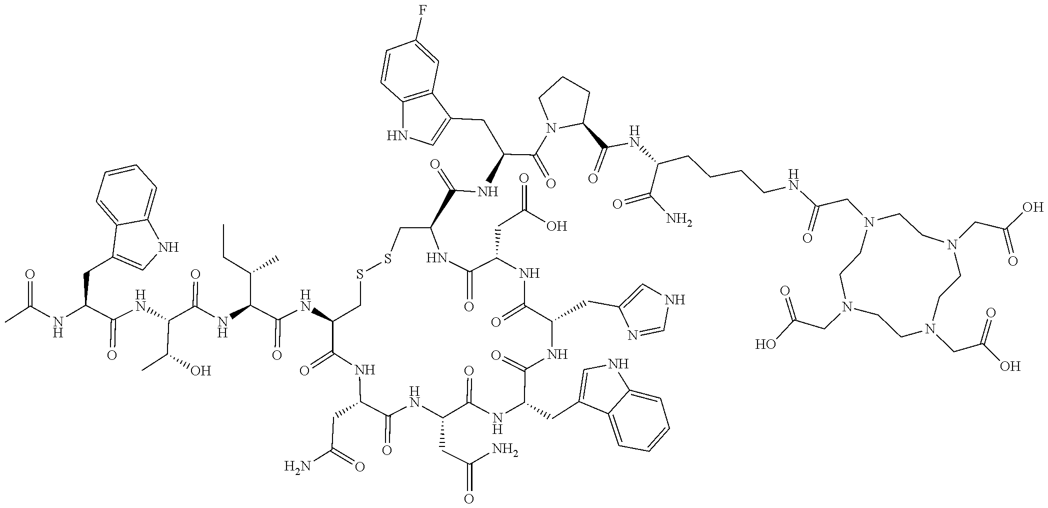 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccc(F)cc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O] | 102 | |
| C₉₆H₁₂₉FN₂₆O₂₆S₂ | | |
| 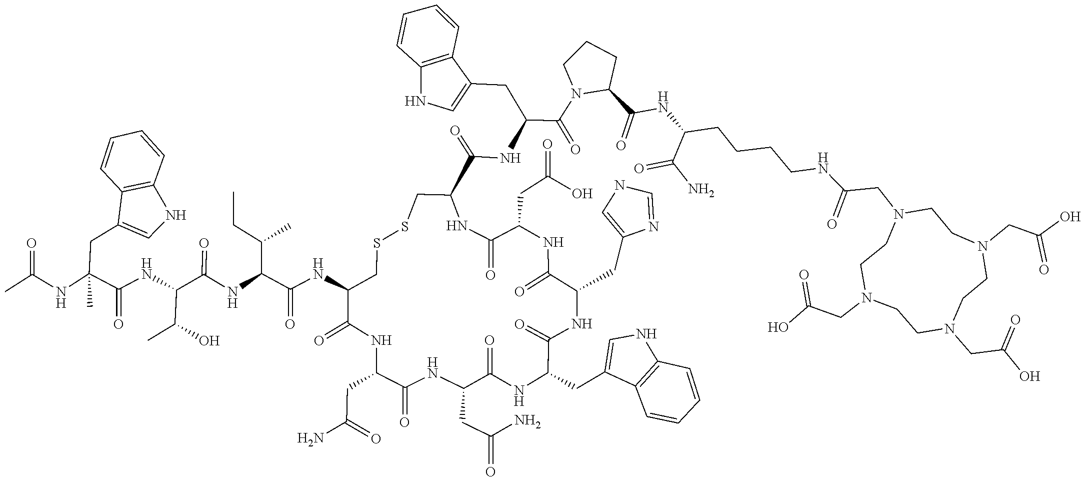 | 103 | |
| C₉₇H₁₃₂N₂₆O₂₆S₂ | | |
| 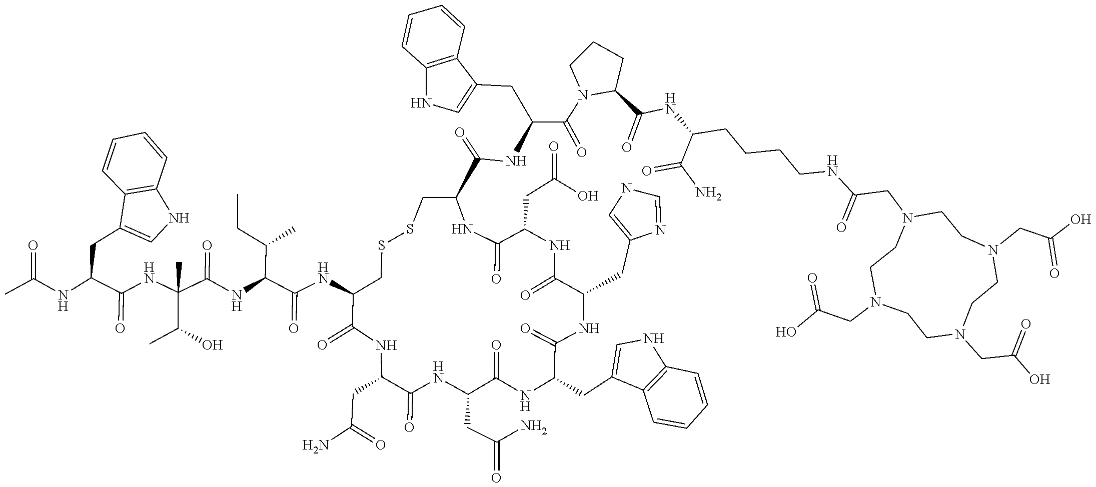 | 104 | |
| C₉₇H₁₃₂N₂₆O₂₆S₂ | | |
| 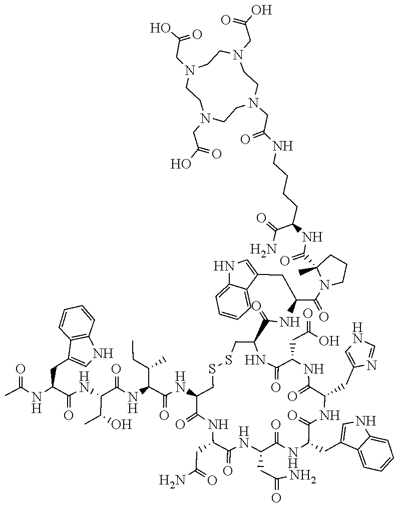 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@@]2(C)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O] | 105 | 109 |
| C₉₇H₁₃₂N₂₆O₂₆S₂ | | |
| 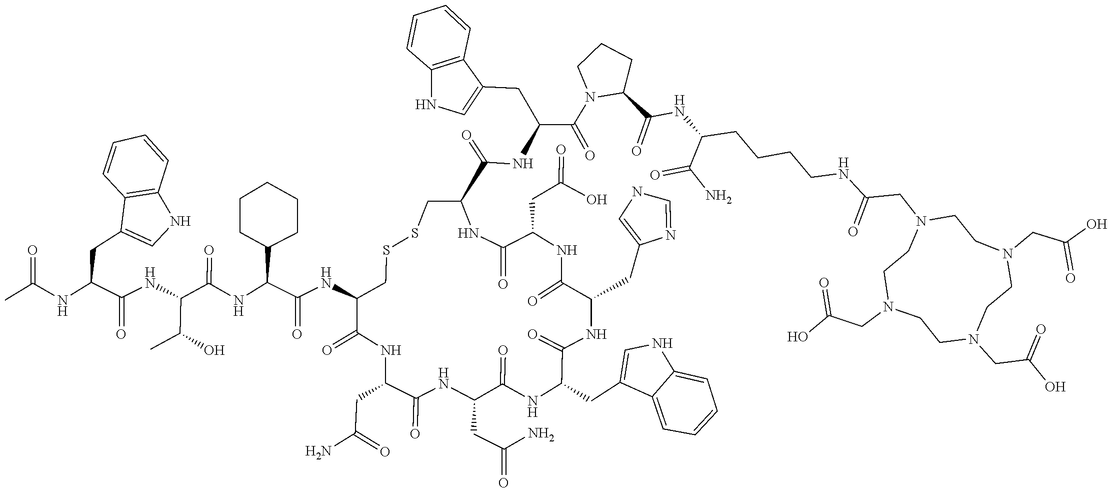 | 106 | 110 |
| C₉₈H₁₃₂N₂₆O₂₆S₂ | | |
| 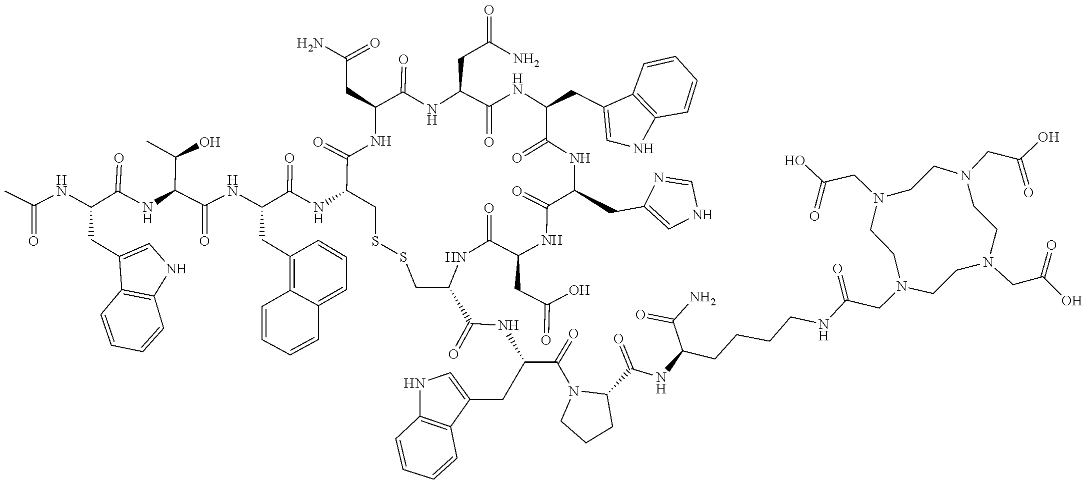 [SMILES: CC(=O)N[C@@H](Cc1c[nH]c2ccccc12)C(=O)N[C@H](C(=O)N[C@@H](Cc1cccc2ccccc12)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O)[C@@H](C)O] | 107 | 111 |
| C₁₀₃H₁₃₀N₂₆O₂₆S₂ | | |
| 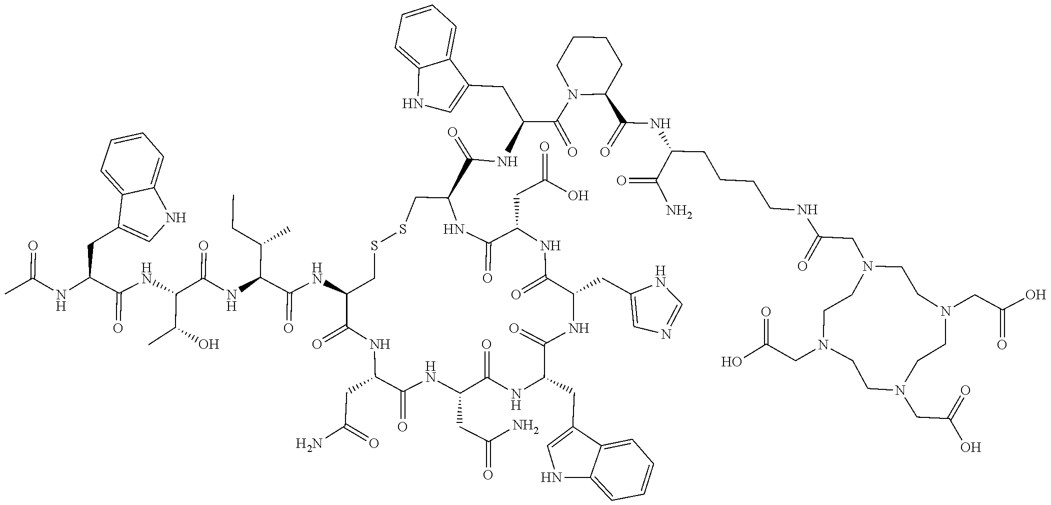 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 108 | 112 |
| C₉₇H₁₃₂N₂₆O₂₆S₂ | | |
| 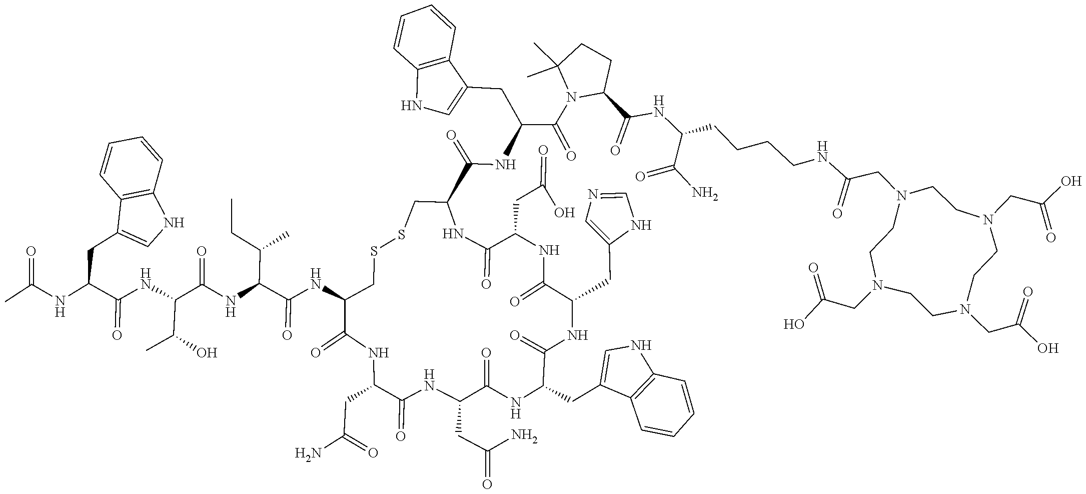 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2[C@H](C(=O)N[C@H](CCCCNC(=O)CN3CCN(CC(=O)O)CCN(CC(=O)O)CCN(CC(=O)O)CC3)C(N)=O)CCC2(C)C)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 109 | 113 |
| C₉₈H₁₃₄N₂₆O₂₆S₂ | | |
| 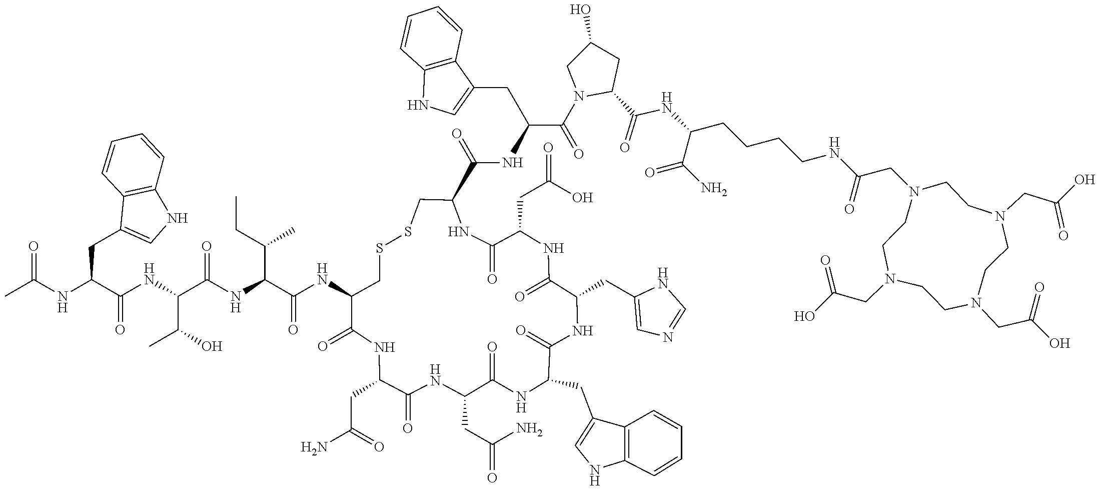 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@H](O)C[C@@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 110 | 114 |
| C₉₆H₁₃₀N₂₆O₂₇S₂ | | |
| 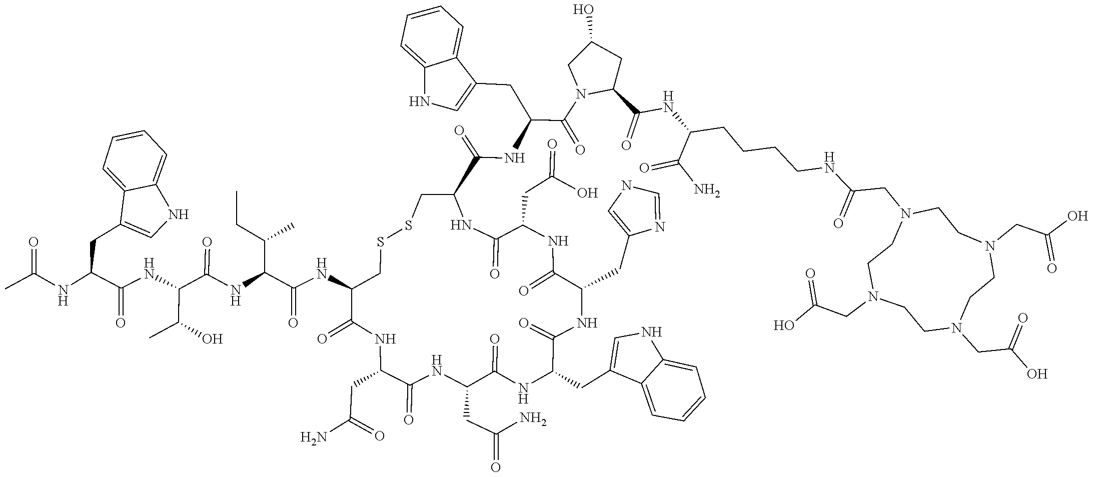 | 111 | 115 |
| C₉₆H₁₃₀N₂₆O₂₇S₂ | | |
| 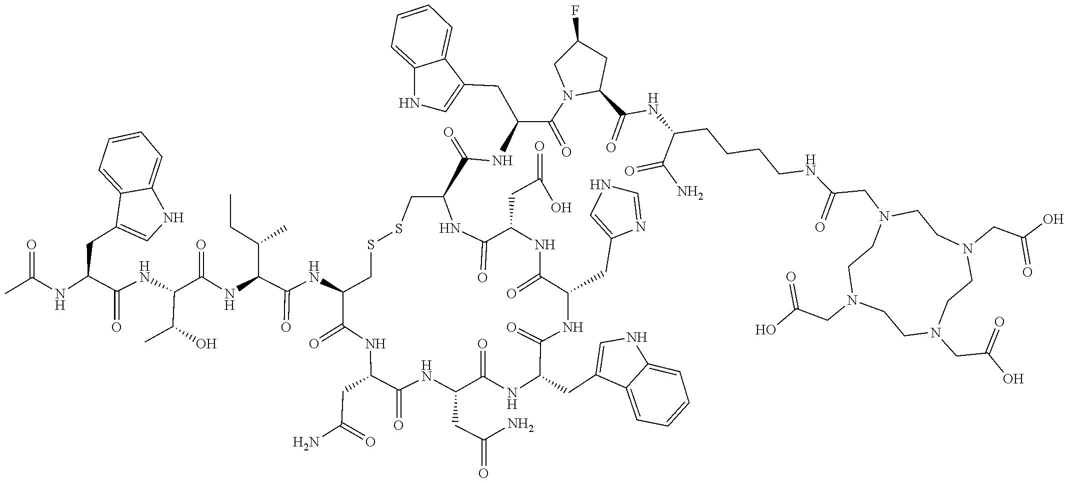 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O] | 112 | 116 |
| C₉₆H₁₂₉FN₂₆O₂₆S₂ | | |
| 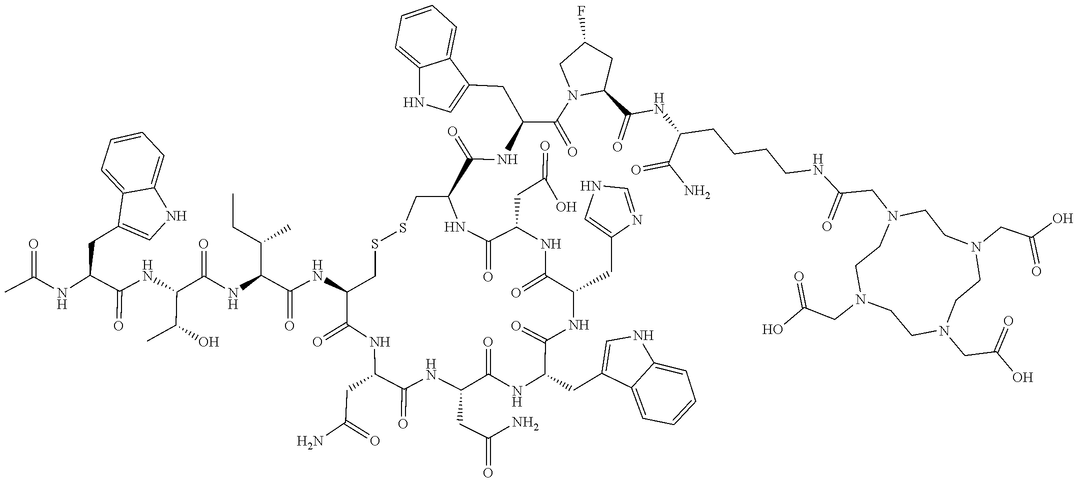 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O] | 113 | 117 |
| C₉₆H₁₂₉FN₂₆O₂₆S₂ | | |
| 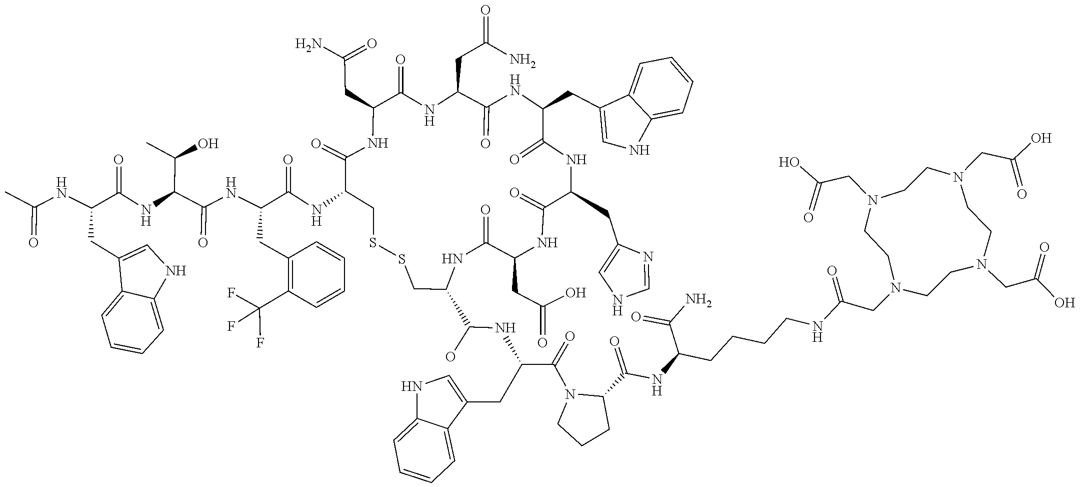 | 114 | 118 |
| C₁₀₀H₁₂₇F₃N₂₆O₂₆S₂ | | |
| 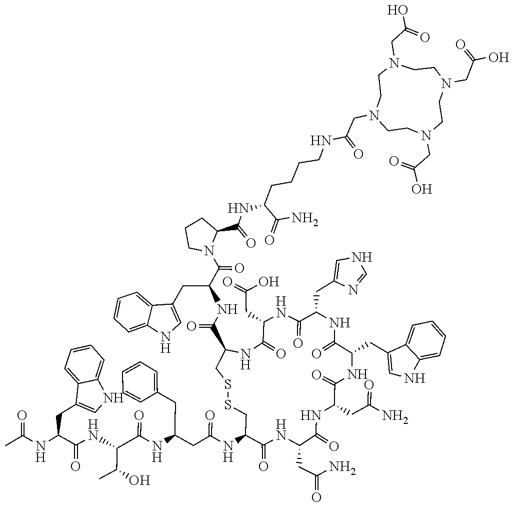 [SMILES: CC(=O)N[C@@H](Cc1c[nH]c2ccccc12)C(=O)N[C@H](C(=O)N[C@H](CC(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O)Cc1ccccc1)[C@@H](C)O] | 115 | 119 |
| C₁₀₀H₁₃₀N₂₆O₂₆S₂ | | |
| 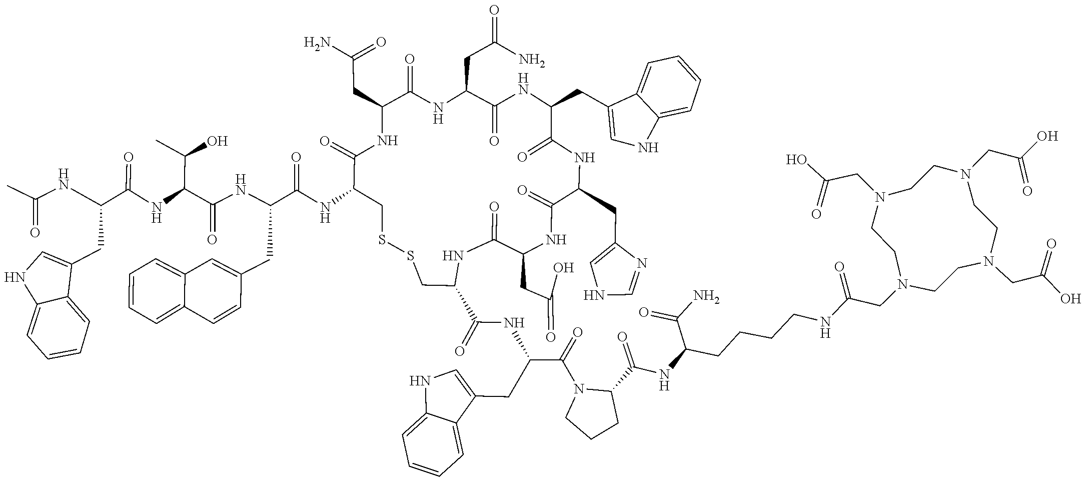 [SMILES: CC(=O)N[C@@H](Cc1c[nH]c2ccccc12)C(=O)N[C@H](C(=O)N[C@@H](Cc1ccc2ccccc2c1)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O)[C@@H](C)O] | 116 | 120 |
| C₁₀₃H₁₃₀N₂₆O₂₆S₂ | | |
| 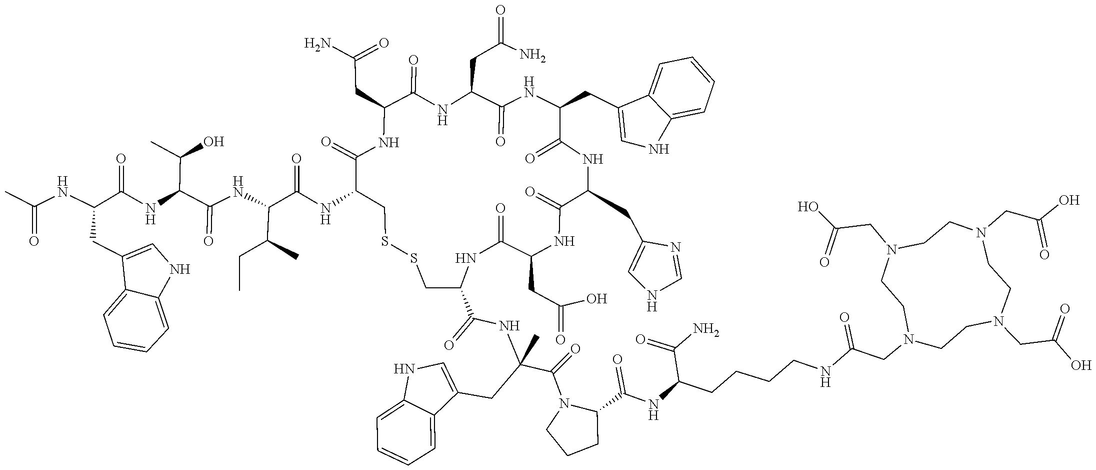 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@](C)(Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O] | 117 | 121 |
| C₉₇H₁₃₂N₂₆O₂₆S₂ | | |
| 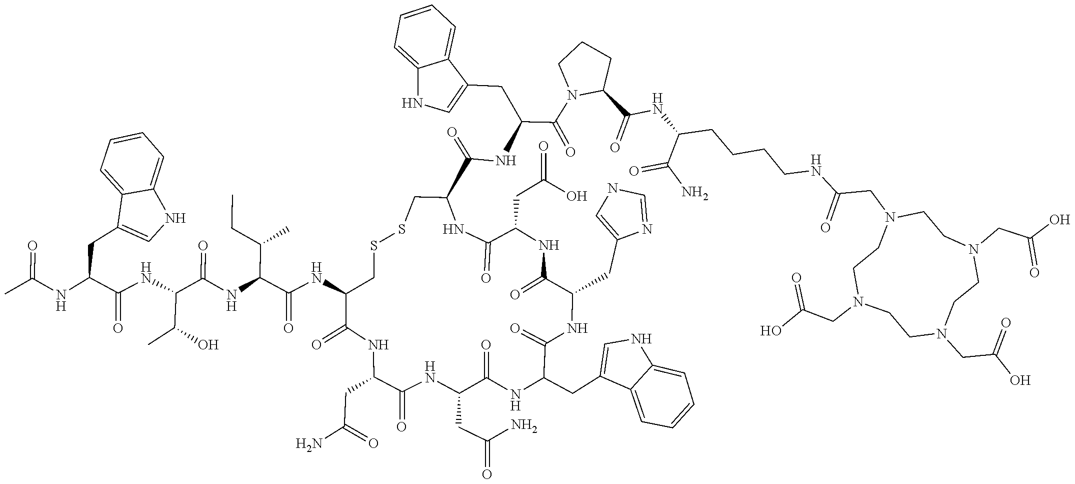 | 118 | 122 |
| C₉₇H₁₃₂N₂₆O₂₆S₂ | | |
| 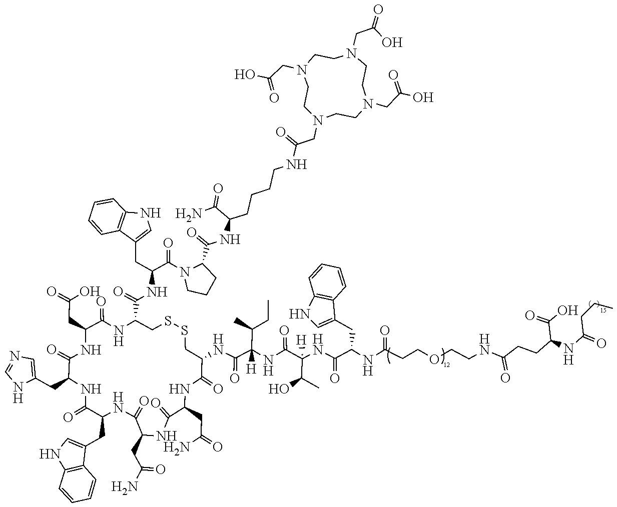 | 119 | 123 |
| C₁₄₂H₂₁₈N₂₈O₄₂S | | |
| 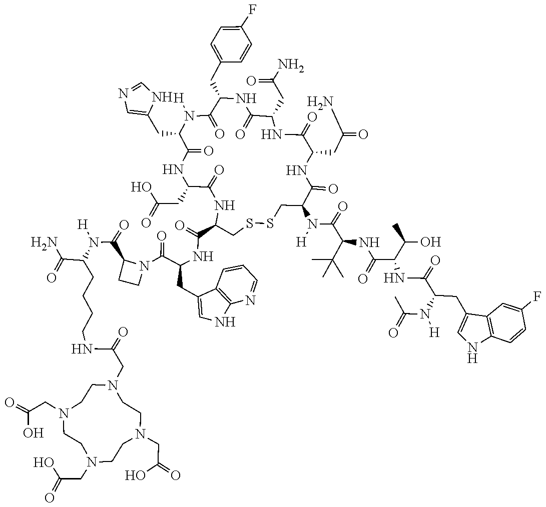 | 121 | 124 |
| C₉₂H₁₂₄F₂N₂₆O₂₆S₂ | | |
| 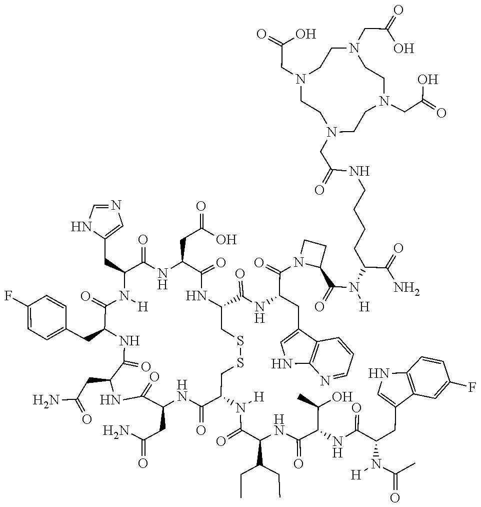 | 122 | 125 |
| C₉₃H₁₂₆F₂N₂₆O₂₆S₂ | | |
| 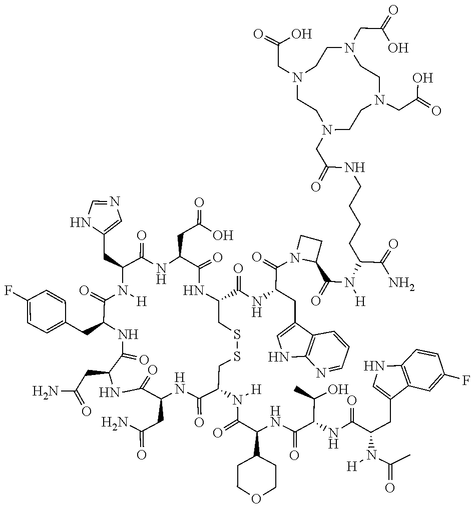 | 123 | 126 |
| C₉₃H₁₂₄F₂N₂₆O₂₇S₂ | | |
| 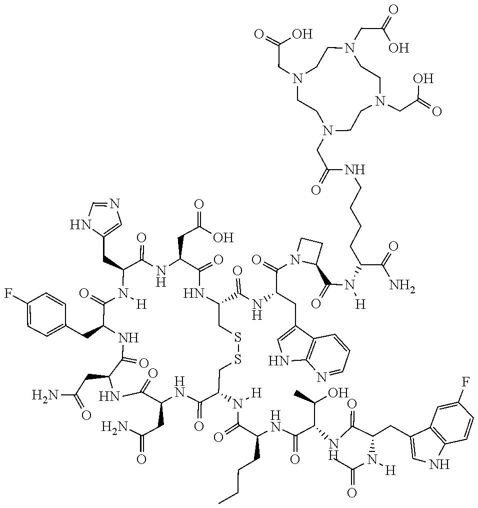 | 124 | 127 |
| C₉₂H₁₂₄F₂N₂₆O₂₆S₂ | | |
| 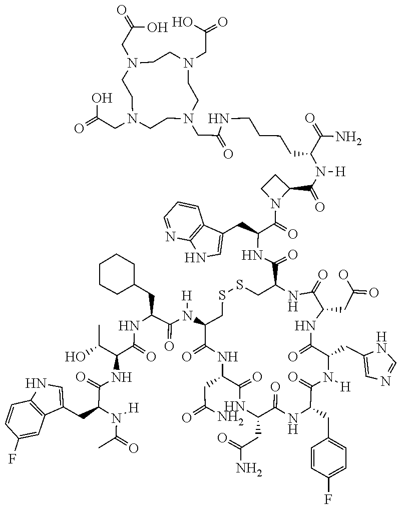 | 125 | 128 |
| C₉₅H₁₂₈F₂N₂₆O₂₆S₂ | | |
| 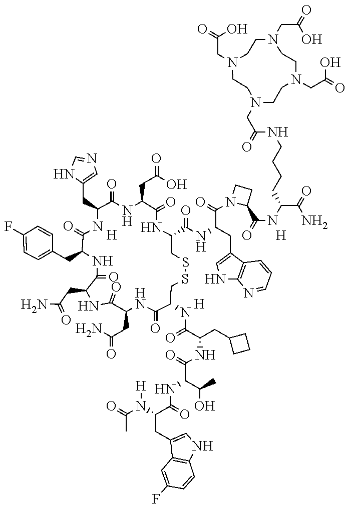 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O] | 126 | 129 |
| C₉₃H₁₂₄F₂N₂₆O₂₆S₂ | | |
| 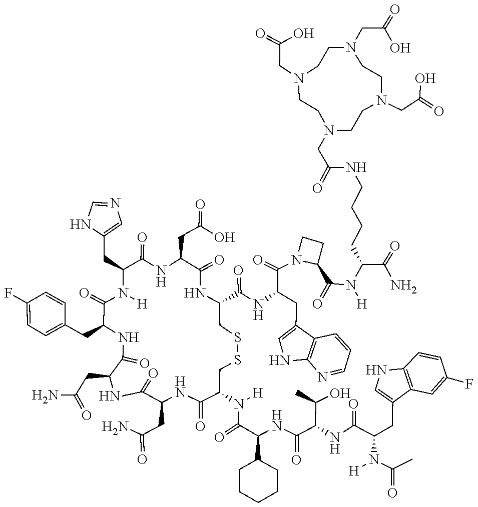 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)C1CCCCC1)[C@@H](C)O] | 127 | 130 |
| C₉₄H₁₂₆F₂N₂₆O₂₆S₂ | | |
| 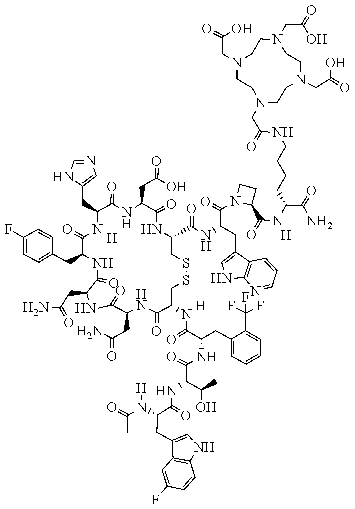 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O] | 128 | 131 |
| C₉₆H₁₂₁F₅N₂₆O₂₆S₂ | | |
| 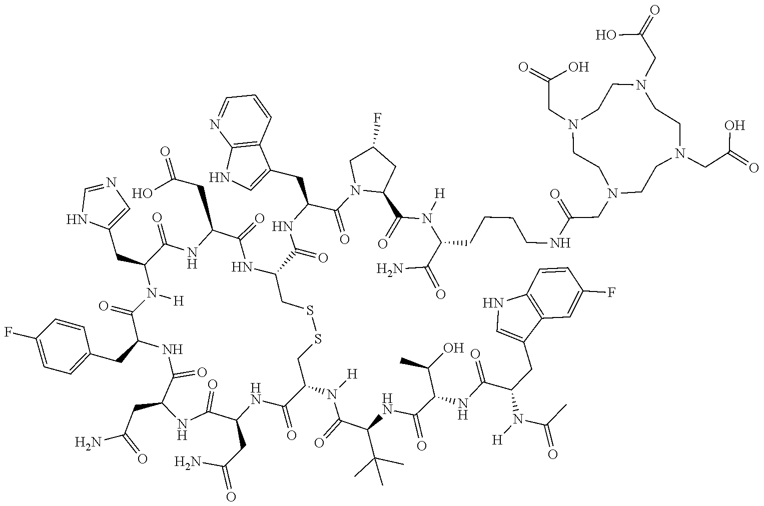 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)C(C)(C)C)[C@@H](C)O] | 129 | 132 |
| C₉₃H₁₂₅F₃N₂₆O₂₆S₂ | | |
| 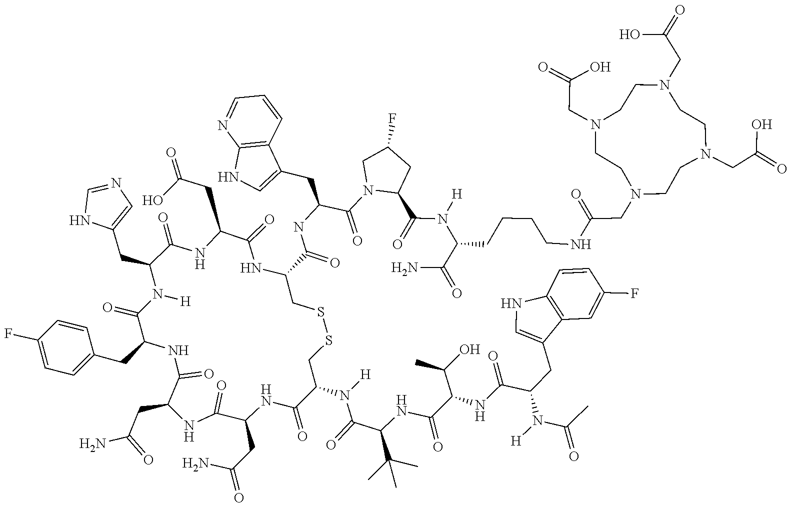 | 130 | 133 |
| C₉₄H₁₂₇F₃N₂₆O₂₆S₂ | | |
| 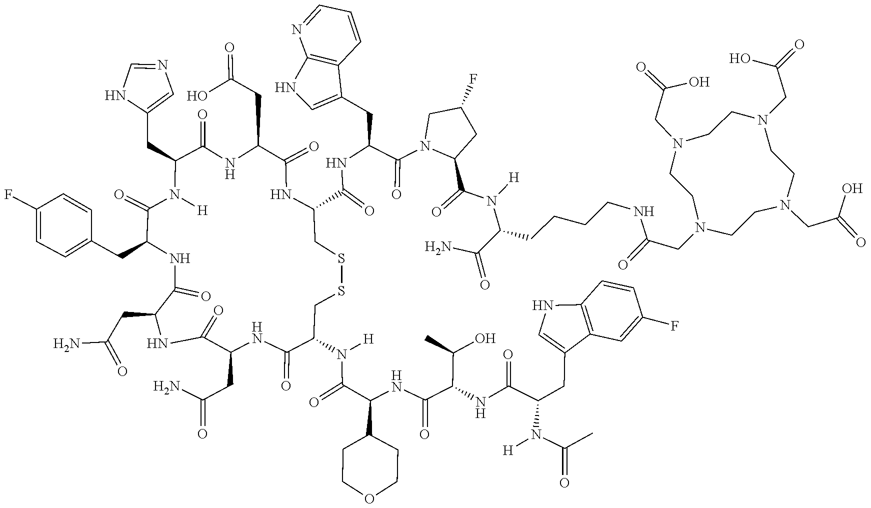 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)C1CCOCC1)[C@@H](C)O] | 131 | 134 |
| C₉₄H₁₂₅F₃N₂₆O₂₇S₂ | | |
| 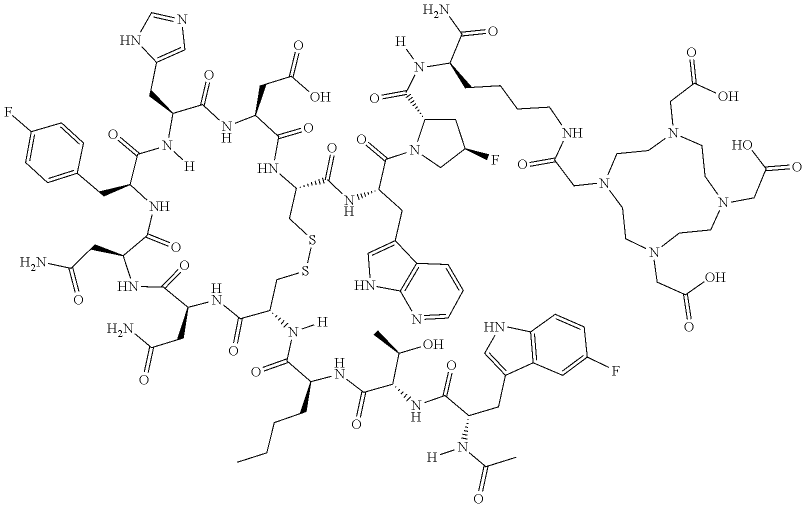 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O] | 132 | 135 |
| C₉₃H₁₂₅F₃N₂₆O₂₆S₂ | | |
| 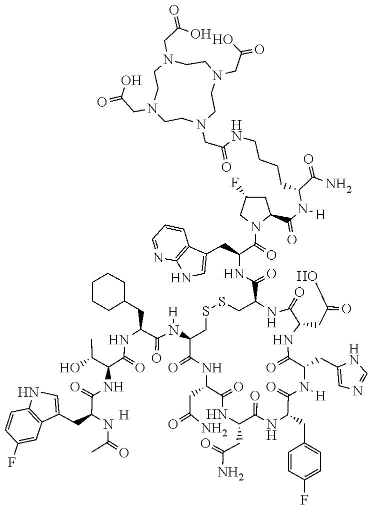 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O] | 133 | 136 |
| C₉₆H₁₂₉F₃N₂₆O₂₆S₂ | | |
| 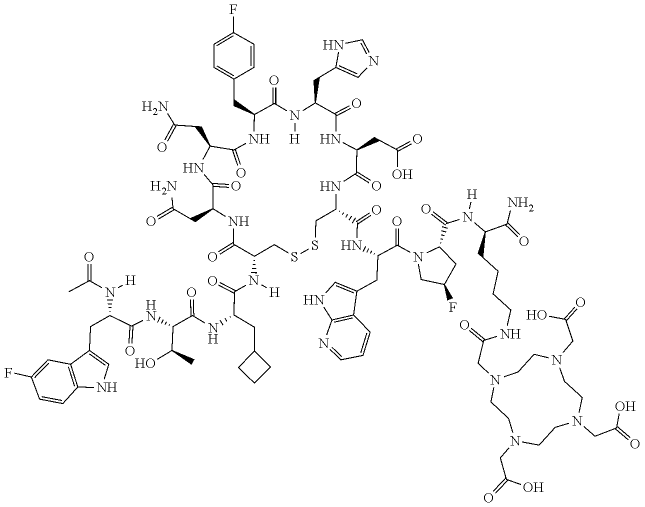 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O] | 134 | 137 |
| C₉₄H₁₂₅F₃N₂₆O₂₆S₂ | | |
| 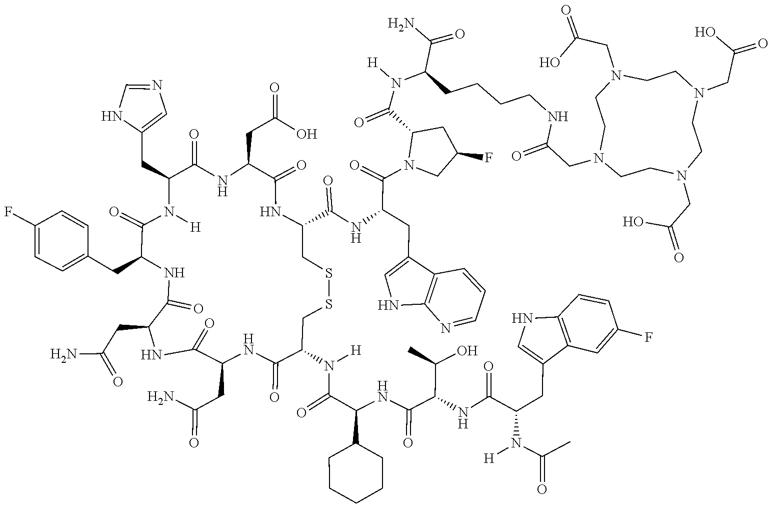 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)C1CCCCC1)[C@@H](C)O] | 135 | 138 |
| C₉₅H₁₂₇F₃N₂₆O₂₆S₂ | | |
| 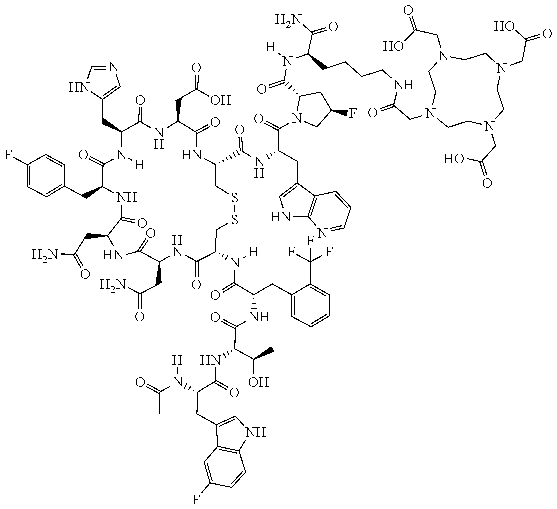 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O] | 136 | 139 |
| C₉₇H₁₂₂F₆N₂₆O₂₆S₂ | | |
| 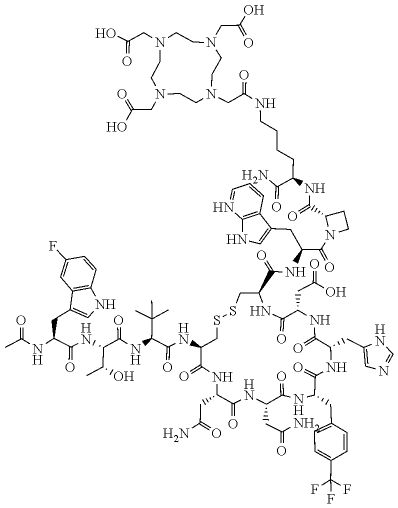 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C(C)(C)C)[C@@H](C)O] | 137 | 140 |
| C₉₃H₁₂₄F₄N₂₆O₂₆S₂ | | |
| 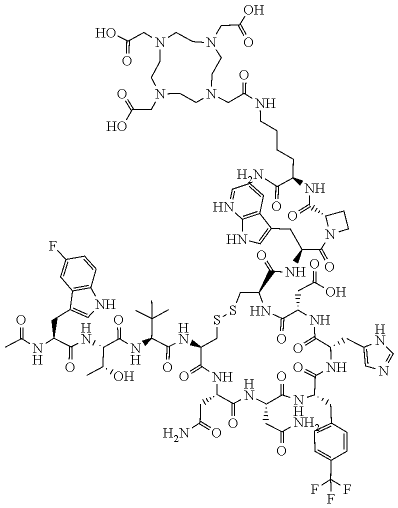 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C(C)(C)C)[C@@H](C)O] | 138 | 141 |
| C₉₄H₁₂₆F₄N₂₆O₂₆S₂ | | |
| 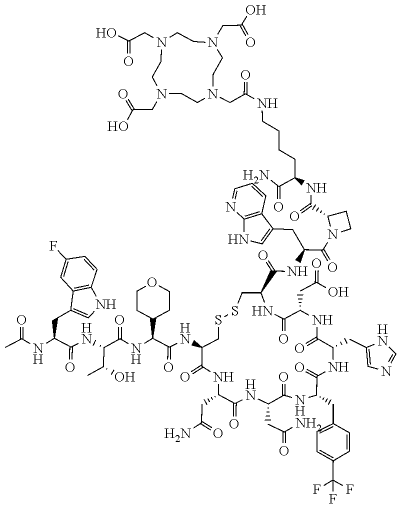 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C1CCOCC1)[C@@H](C)O] | 139 | 142 |
| C₉₄H₁₂₄F₄N₂₆O₂₇S₂ | | |
| 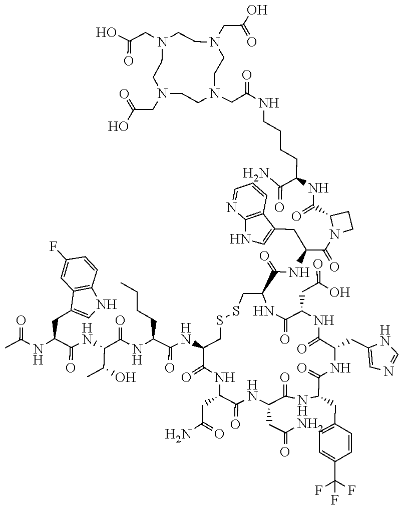 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O] | 140 | 143 |
| C₉₃H₁₂₄F₄N₂₆O₂₆S₂ | | |
| 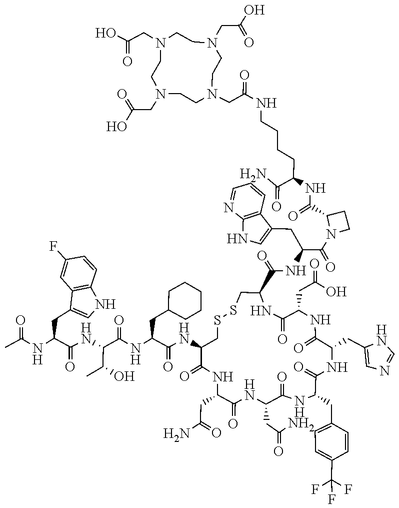 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O] | 141 | 144 |
| C₉₆H₁₂₈F₄N₂₆O₂₆S₂ | | |
| 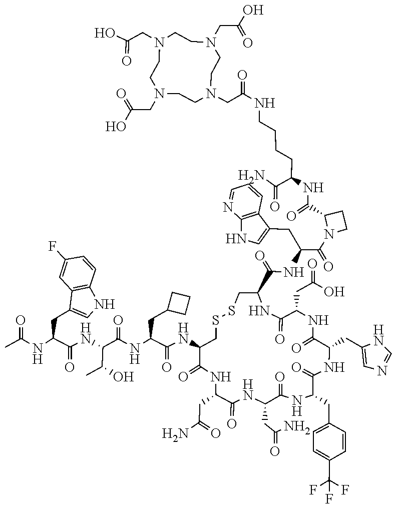 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O] | 142 | 145 |
| C₉₄H₁₂₄F₄N₂₆O₂₆S₂ | | |
| 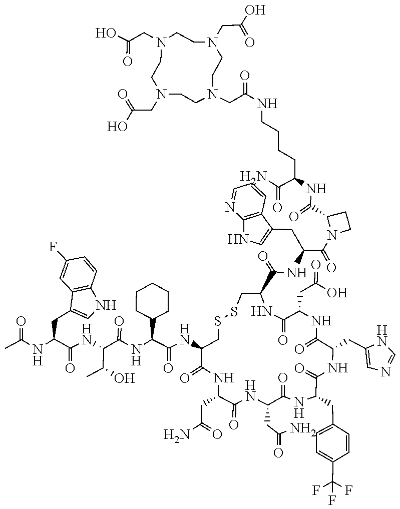 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C1CCCCC1)[C@@H](C)O] | 143 | 146 |
| C₉₅H₁₂₆F₄N₂₆O₂₆S₂ | | |
| 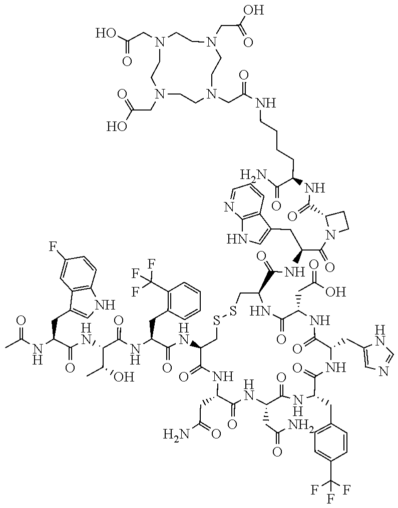 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O] | 144 | 147 |
| C₉₇H₁₂₁F₇N₂₆O₂₆S₂ | | |
| 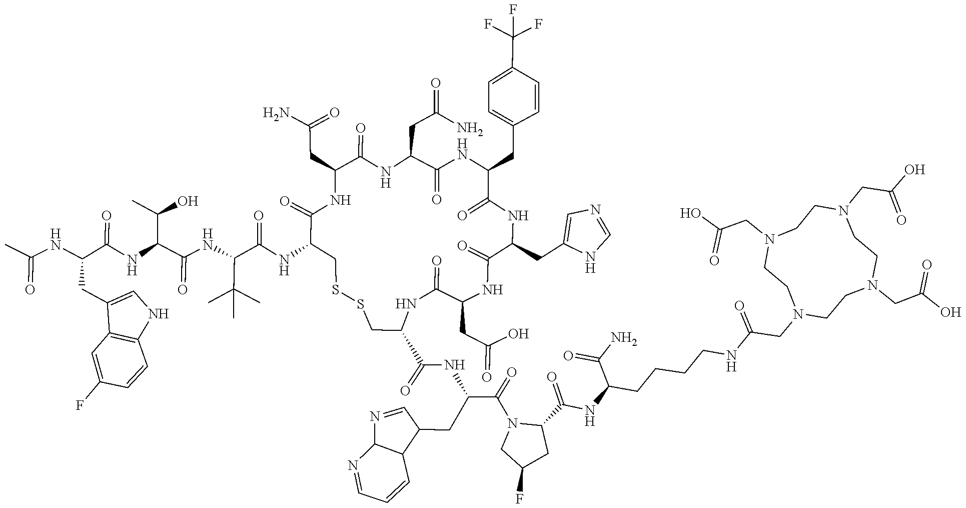 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](CC2C=NC3N=CC=CC23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C(C)(C)C)[C@@H](C)O] | 145 | 148 |
| C₉₄H₁₂₇F₅N₂₆O₂₆S₂ | | |
| 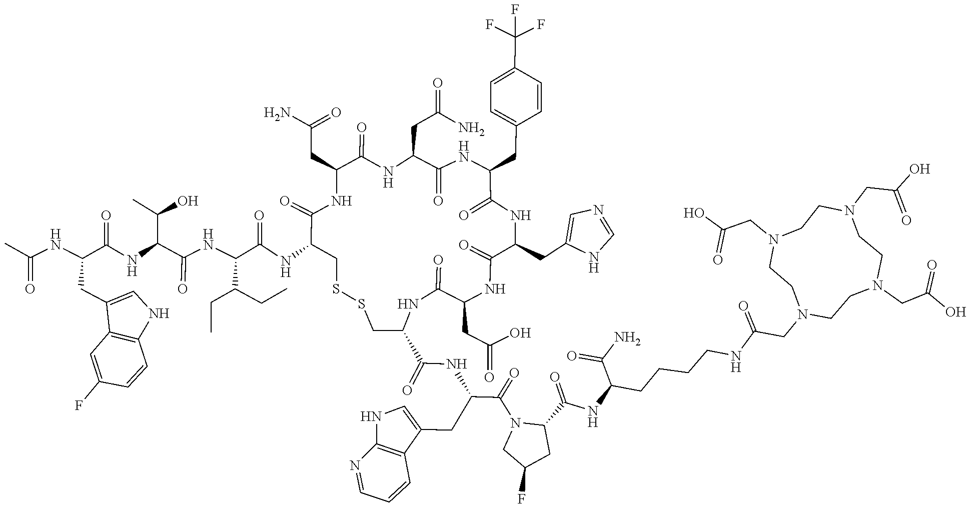 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O] | 146 | 149 |
| C₉₅H₁₂₇F₅N₂₆O₂₆S₂ | | |
| 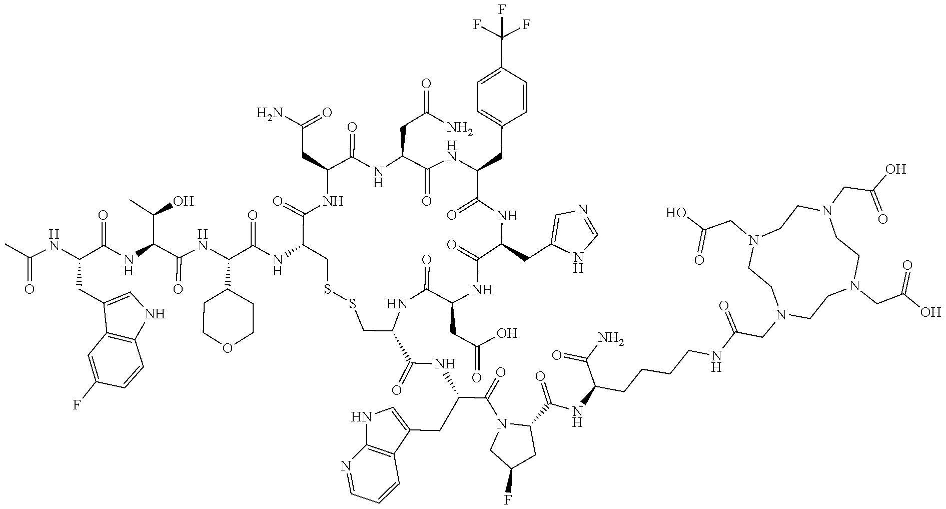 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C1CCOCC1)[C@@H](C)O] | 147 | 150 |
| C₉₅H₁₂₅F₅N₂₆O₂₇S₂ | | |
| 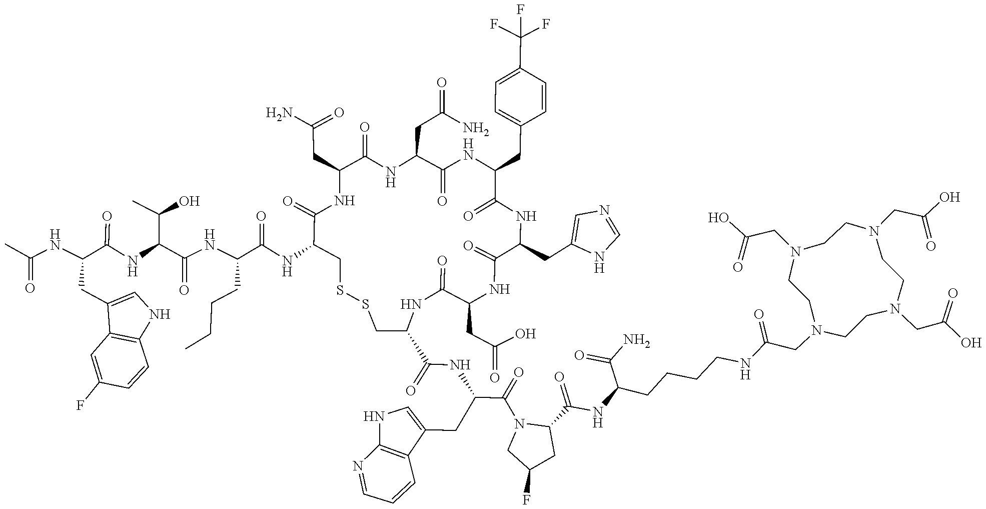 | 148 | 151 |
| C₉₄H₁₂₅F₅N₂₆O₂₆S₂ | | |
| 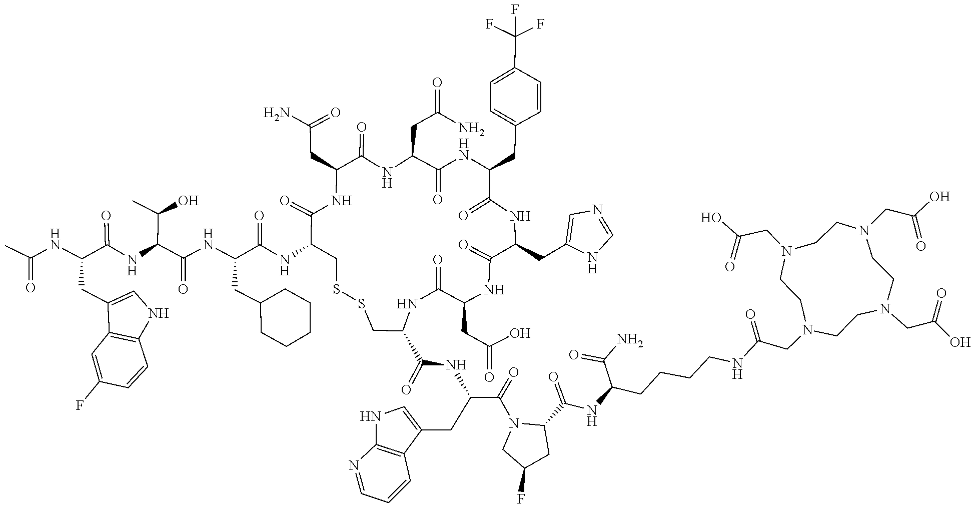 | 149 | 152 |
| C₉₇H₁₂₉F₅N₂₆O₂₆S₂ | | |
| 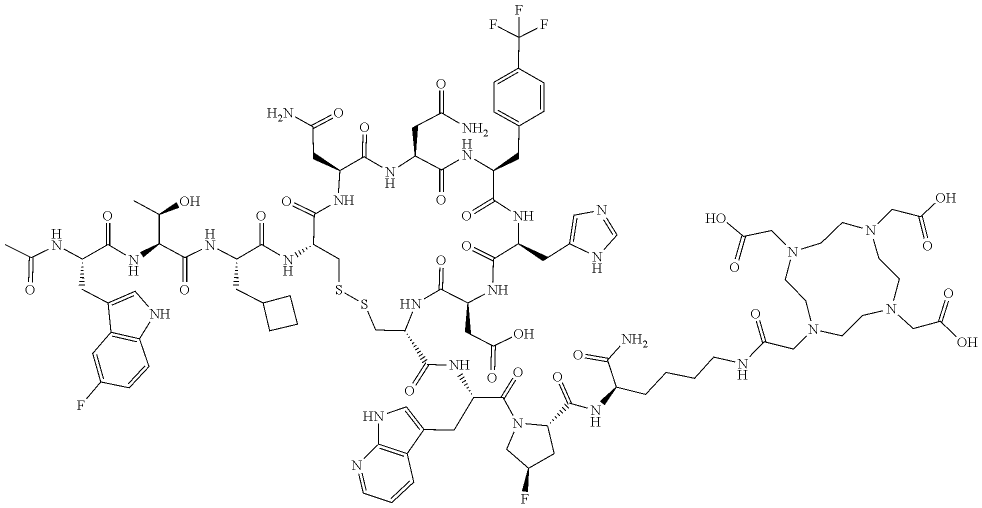 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O] | 150 | 153 |
| C₉₆H₁₂₆F₅N₂₆O₂₆S₂ | | |
| 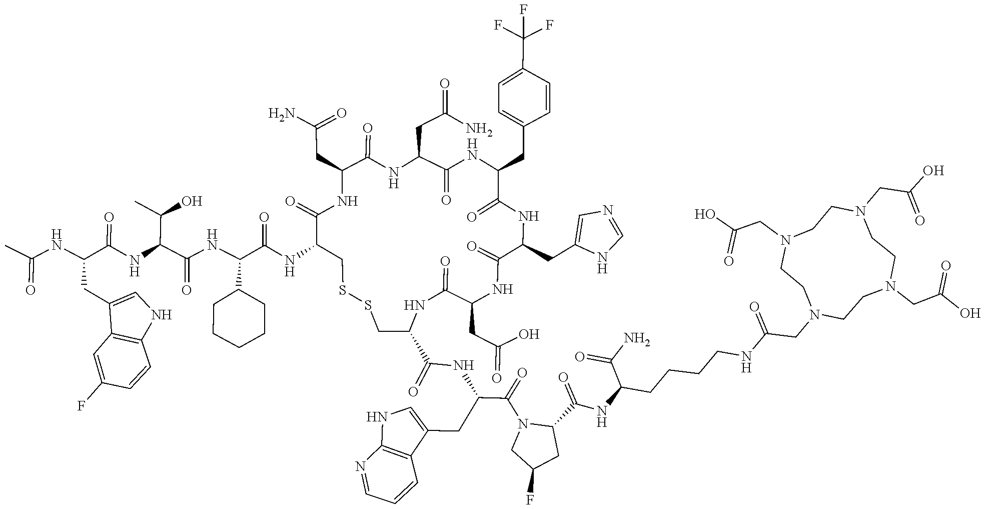 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C1CCCCC1)[C@@H](C)O] | 151 | 154 |
| C₉₆H₁₂₇F₅N₂₆O₂₆S₂ | | |
| 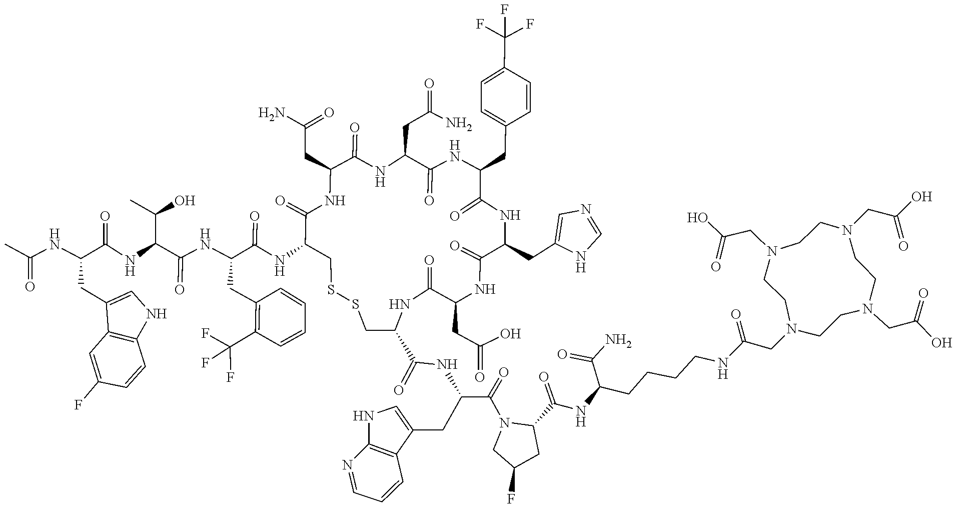 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O] | 152 | 155 |
| C₉₈H₁₂₂F₈N₂₆O₂₆S₂ | | |
| 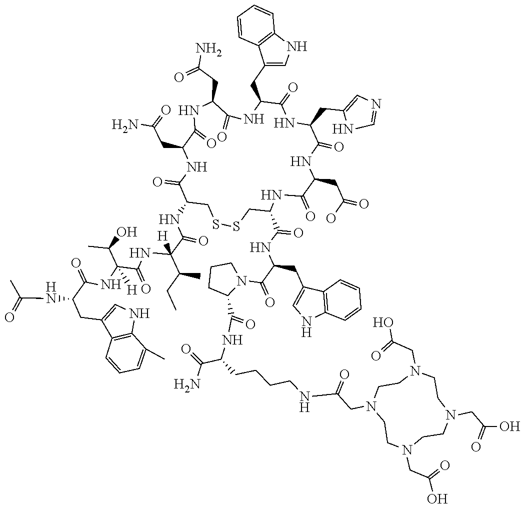 | 156 | 156 |
| C₉₇H₁₃₂N₂₆O₂₆S₂ | | |
| 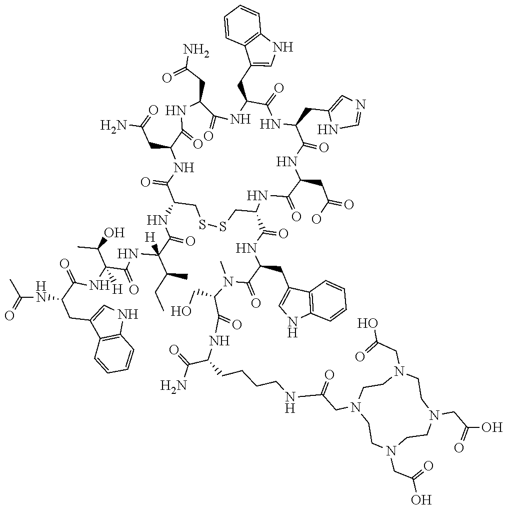 | 157 | 157 |
| C₉₅H₁₃₀N₂₆O₂₇S₂ | | |
| 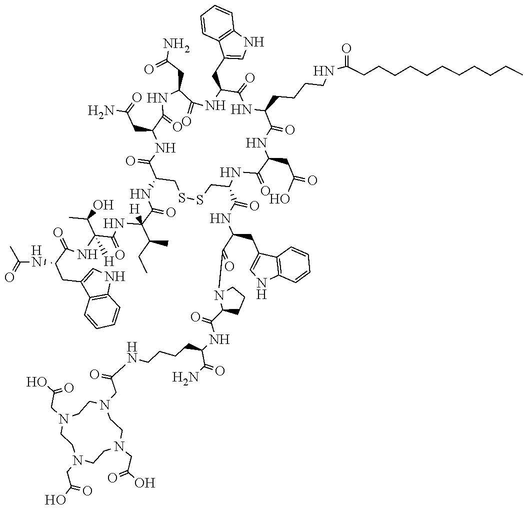 [SMILES: CCCCCCCCCCCC(=O)NCCCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O] | 158 | 158 |
| C₁₀₈H₁₅₇N₂₅O₂₇S₂ | | |
| 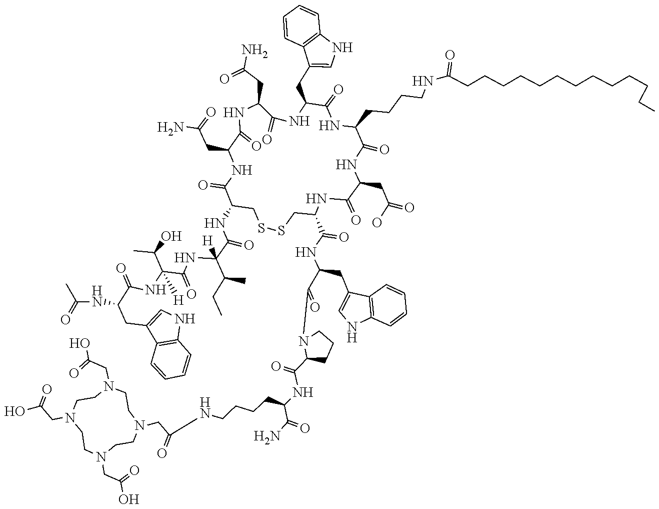 | 159 | 159 |
| C₁₁₀H₁₆₁N₂₅O₂₇S₂ | | |
| 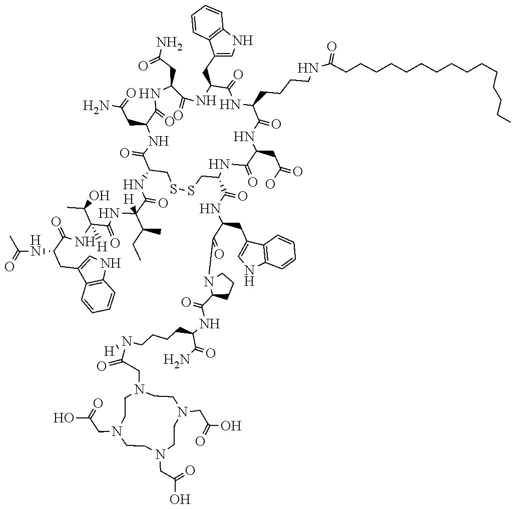 | 160 | 160 |
| C₁₁₂H₁₆₅N₂₅O₂₇S₂ | | |
| 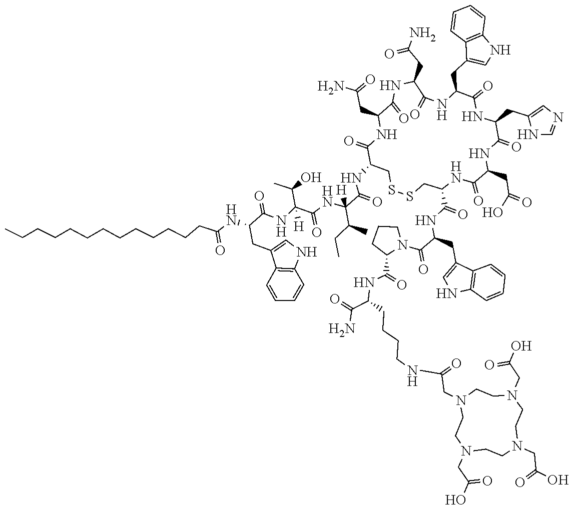 [SMILES: CCCCCCCCCCCCCC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O)[C@@H](C)CC)[C@@H](C)O] | 161 | 161 |
| C₁₀₈H₁₄₃N₂₆O₂₆S₂ | | |
| 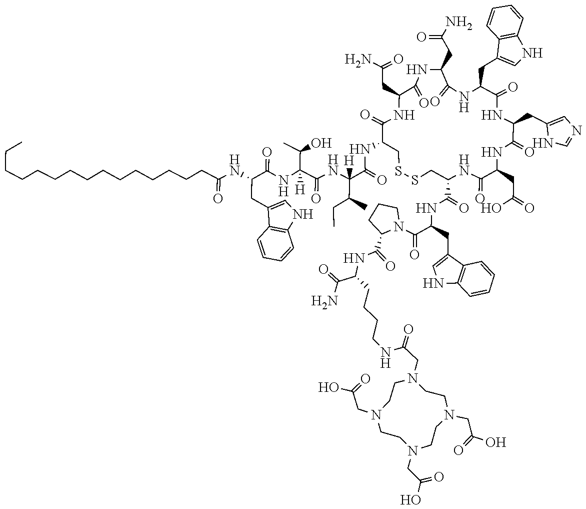 [SMILES: CCCCCCCCCCCCCCCC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O)[C@@H](C)CC)[C@@H](C)O] | 162 | 162 |
| 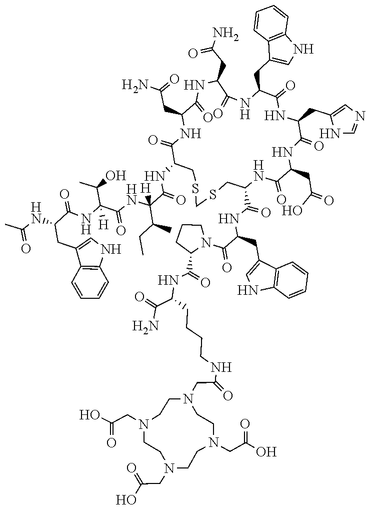 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 164 | 163 |
| C₉₇H₁₃₂N₂₆O₂₆S₂ | | |
| 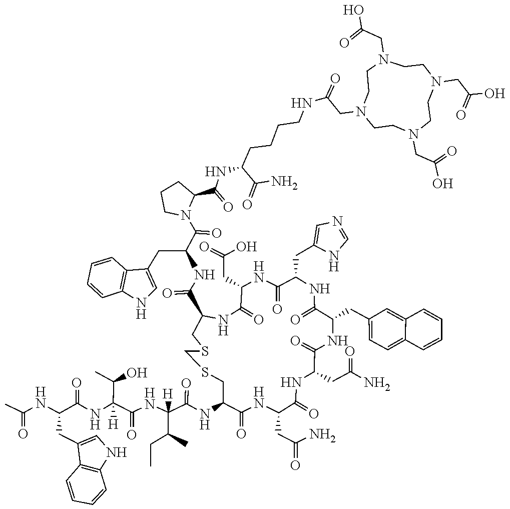 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 165 | 164 |
| C₉₉H₁₃₃N₂₅O₂₆S₂ | | |
| 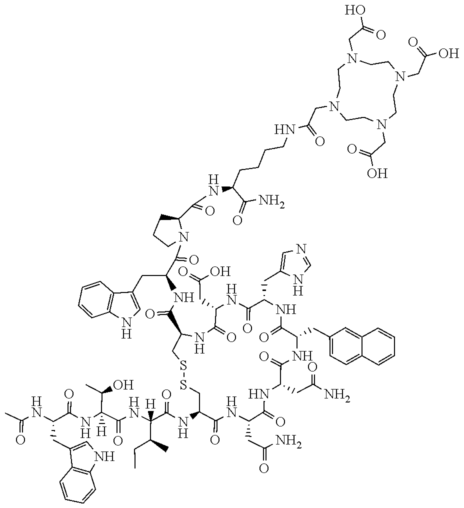 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 166 | 165 |
| C₉₈H₁₃₁N₂₅O₂₆S₂ | | |
| 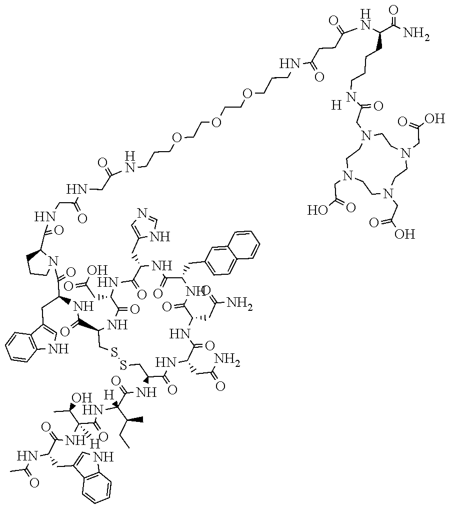 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCC(=O)NCC(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 167 | 166 |
| C₁₁₆H₁₆₃N₂₉O₃₃S₂ | | |
| 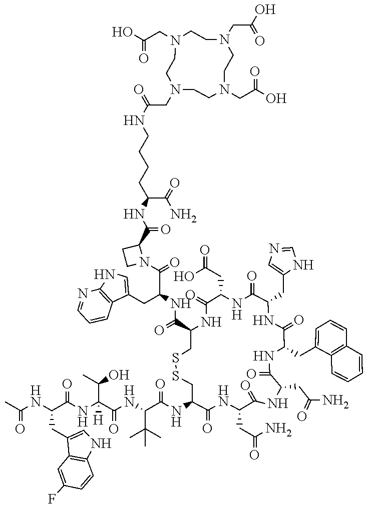 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C(C)(C)C)[C@@H](C)O] | 168 | 167 |
| C₉₆H₁₂₇FN₂₆O₂₆S₂ | | |
| 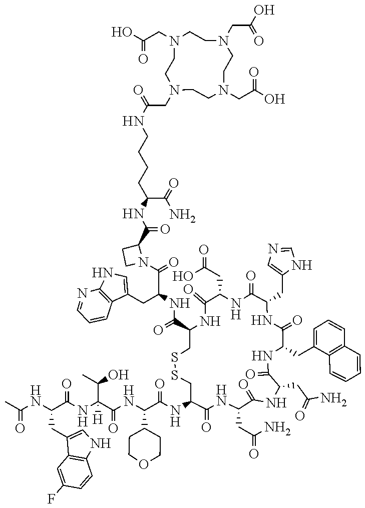 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C1CCOCC1)[C@@H](C)O] | 169 | 168 |
| C₉₇H₁₂₇FN₂₆O₂₇S₂ | | |
| 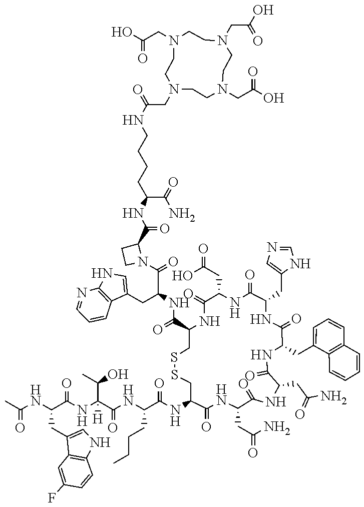 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O] | 170 | 169 |
| C₉₆H₁₂₇FN₂₆O₂₆S₂ | | |
| 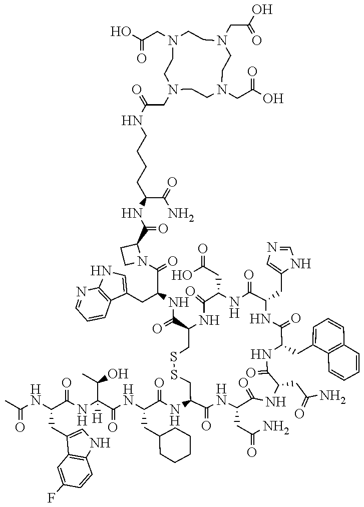 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O] | 171 | 170 |
| C₉₉H₁₃₁FN₂₆O₂₆S₂ | | |
| 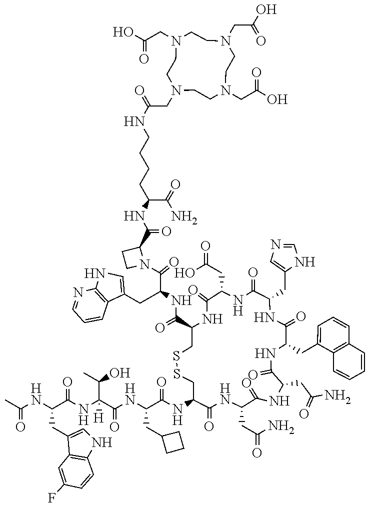 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O] | 172 | 171 |
| C₉₇H₁₂₇FN₂₆O₂₆S₂ | | |
| 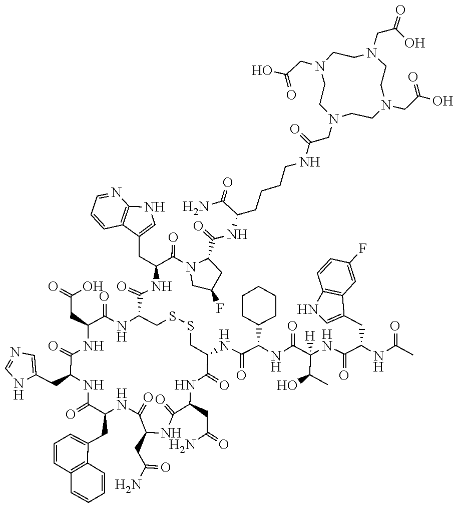 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C1CCCCC1)[C@@H](C)O] | 173 | 172 |
| C₉₉H₁₃₀F₂N₂₆O₂₆S₂ | | |
| 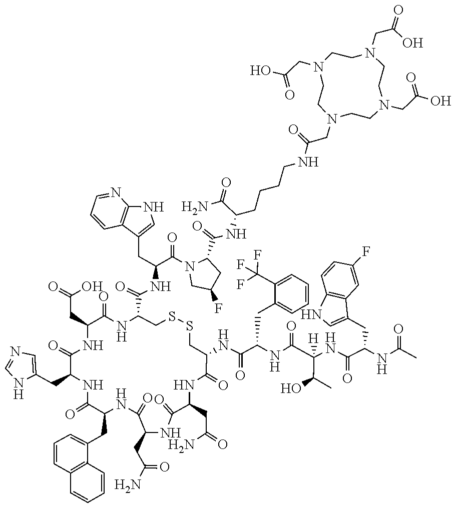 [SMILES: CC(=O)N[C@@H](Cc1c[nH]c2ccc(F)cc12)C(=O)N[C@H](C(=O)N[C@@H](CC1=CCC=CC=C1C(F)(F)F)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O] | 174 | 173 |
| C₁₀₁H₁₂₅F₅N₂₆O₂₆S₂ | | |
| 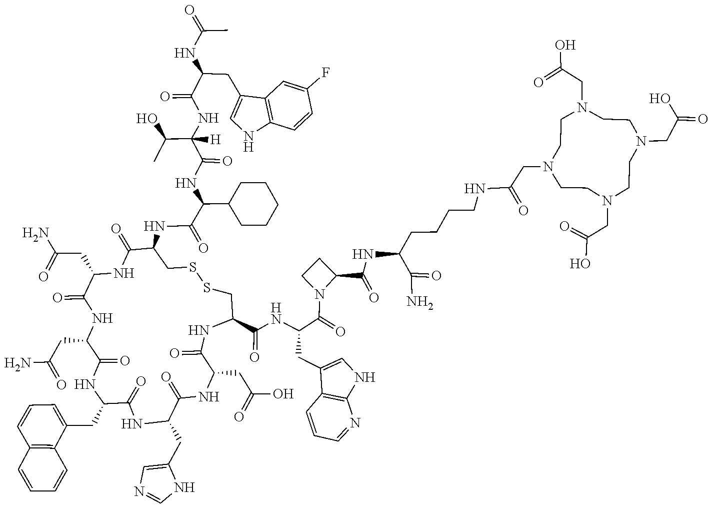 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C1CCCCC1)[C@@H](C)O] | 175 | 174 |
| C₉₈H₁₂₉FN₂₆O₂₆S₂ | | |
| 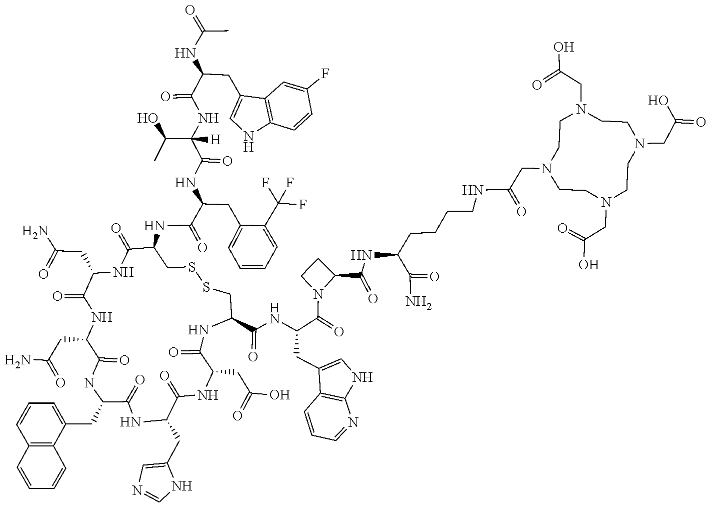 | 176 | 175 |
| C₁₀₀H₁₂₄F₄N₂₆O₂₆S₂ | | |
| 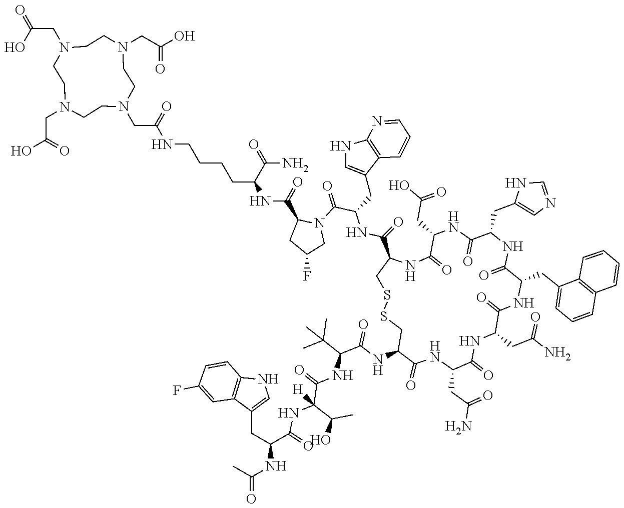 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C(C)(C)C)[C@@H](C)O] | 177 | 176 |
| C₉₇H₁₂₈F₂N₂₆O₂₆S₂ | | |
| 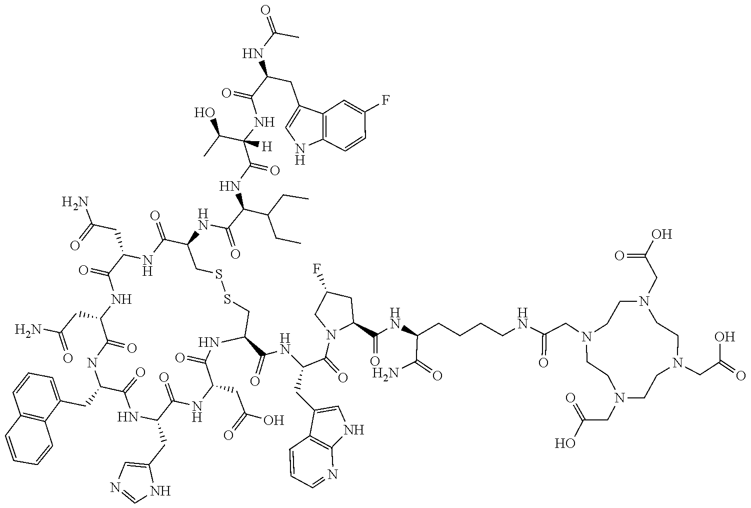 | 178 | 177 |
| C₉₈H₁₃₀F₂N₂₆O₂₆S₂ | | |
| 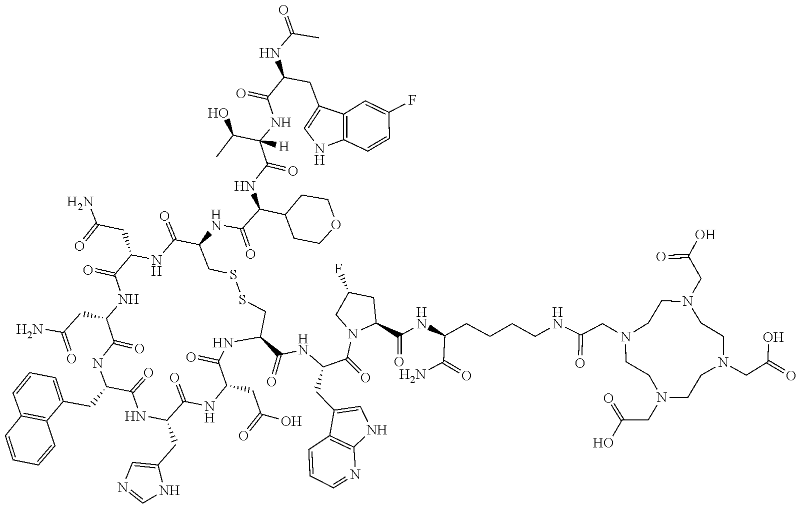 | 179 | 178 |
| C₉₈H₁₂₈F₂N₂₆O₂₇S₂ | | |
| 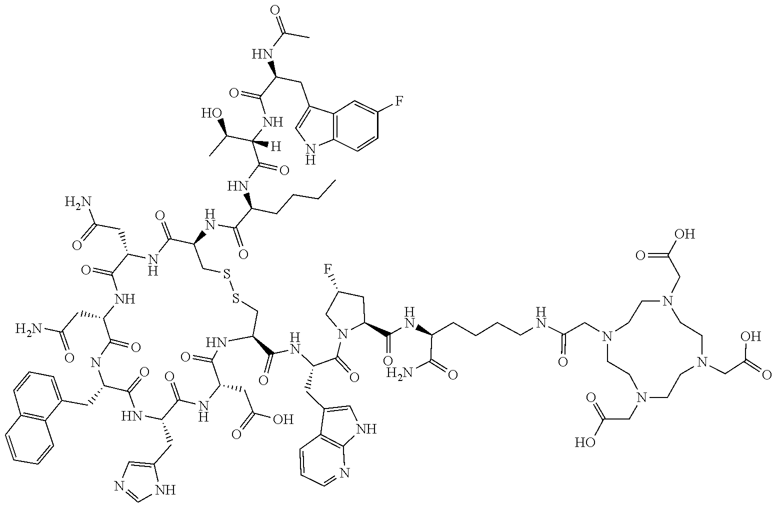 | 180 | 179 |
| C₉₇H₁₂₈F₂N₂₆O₂₆S₂ | | |
| 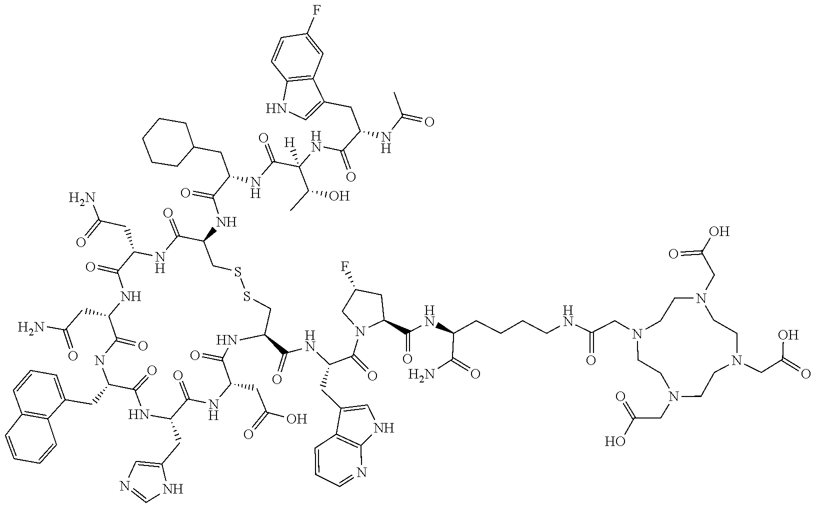 | 181 | 180 |
| C₁₀₀H₁₃₂F₂N₂₆O₂₆S₂ | | |
| 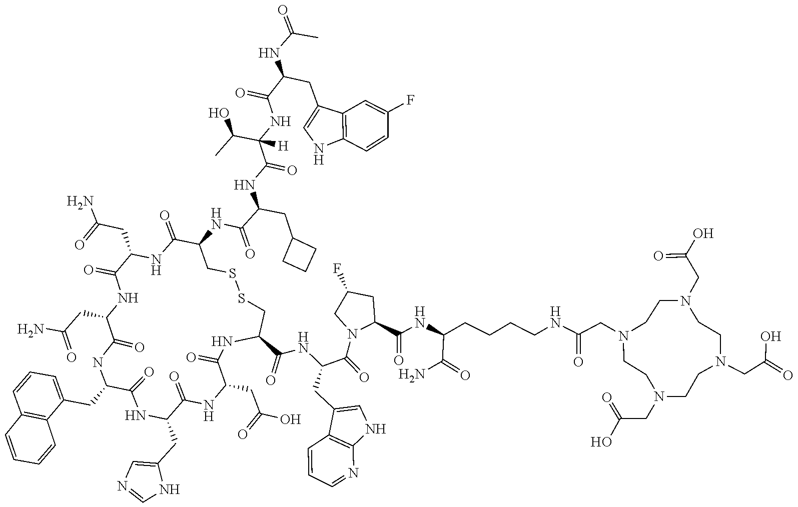 | 182 | 181 |
| C₉₈H₁₂₈F₂N₂₆O₂₆S₂ | | |
| 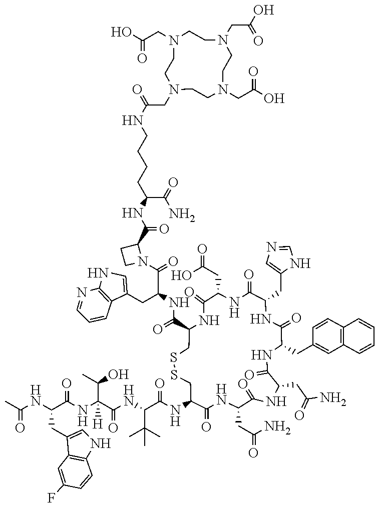 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C(C)(C)C)[C@@H](C)O] | 183 | 182 |
| C₉₆H₁₂₇FN₂₆O₂₆S₂ | | |
| 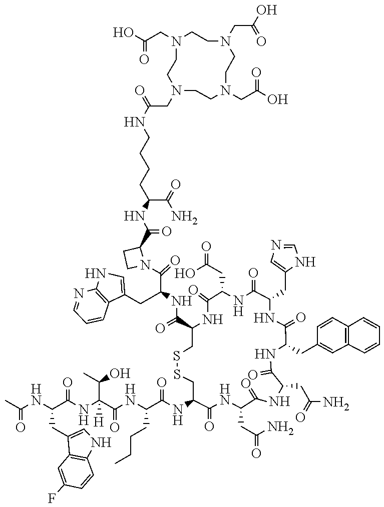 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 184 | 183 |
| C₉₆H₁₂₇FN₂₆O₂₆S₂ | | |
| 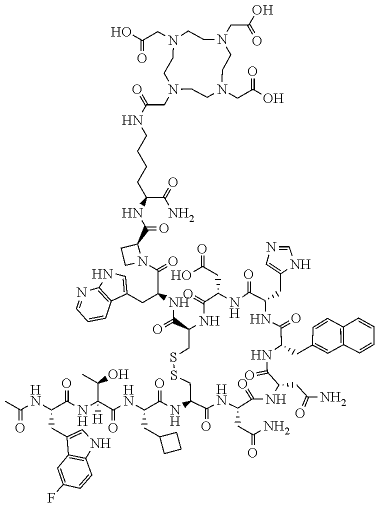 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O] | 185 | 184 |
| C₉₇H₁₂₇FN₂₆O₂₆S₂ | | |
| 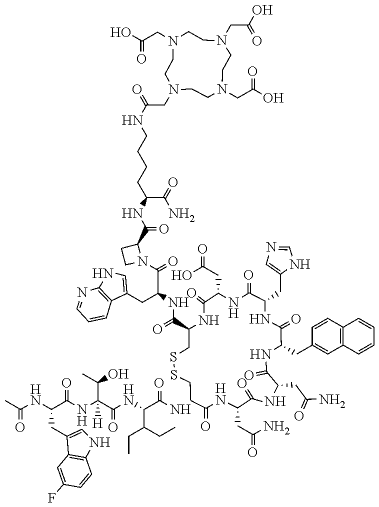 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 186 | 185 |
| C₉₇H₁₂₉FN₂₆O₂₆S₂ | | |
| 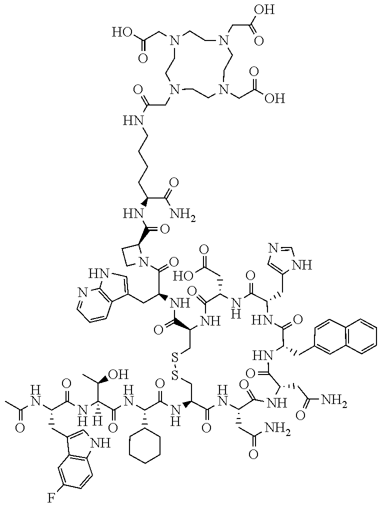 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCCCC1)[C@@H](C)O] | 187 | 186 |
| C₉₈H₁₂₉FN₂₆O₂₆S₂ | | |
| 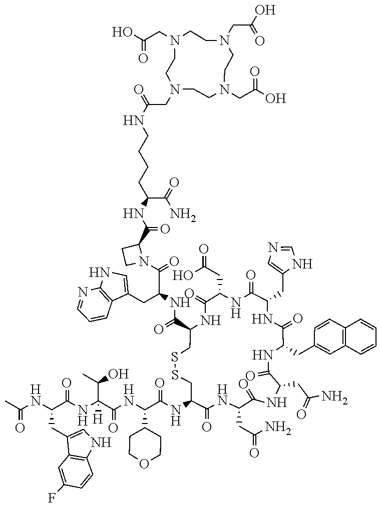 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCOCC1)[C@@H](C)O] | 188 | 187 |
| C₉₇H₁₂₇FN₂₆O₂₇S₂ | | |
| 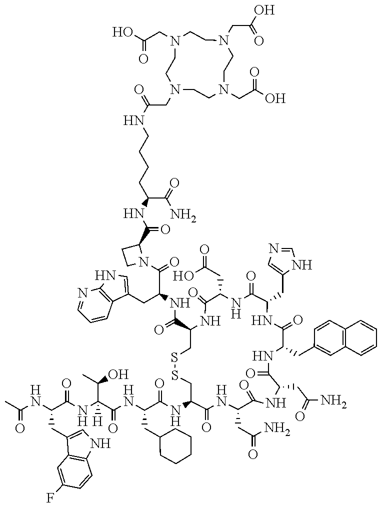 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O] | 189 | 188 |
| C₉₉H₁₃₁FN₂₆O₂₆S₂ | | |
| 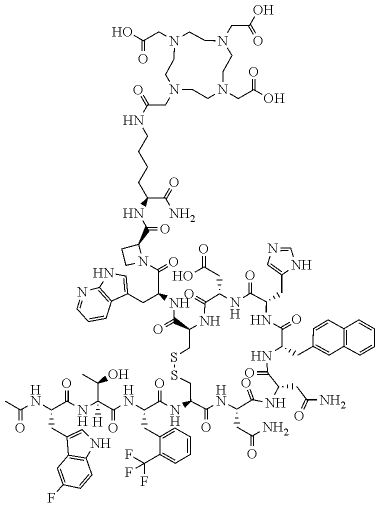 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O] | 190 | 189 |
| C₁₀₀H₁₂₄F₄N₂₆O₂₆S₂ | | |
| 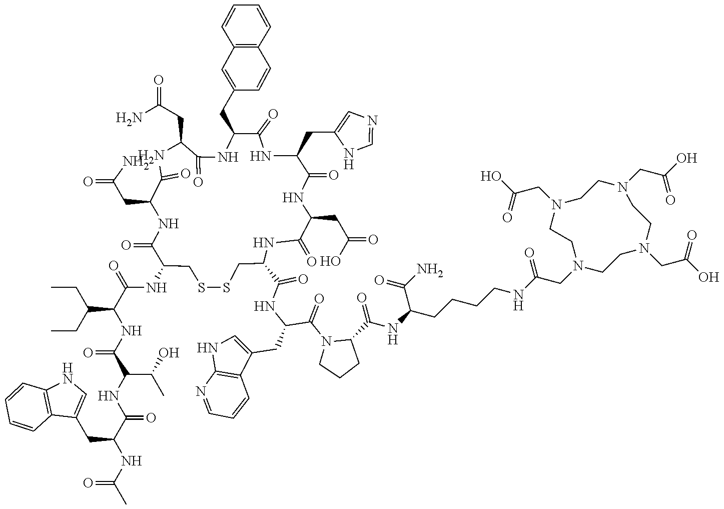 | 193 | 190 |
| C₉₈H₁₃₂N₂₆O₂₆S₂ | | |
| 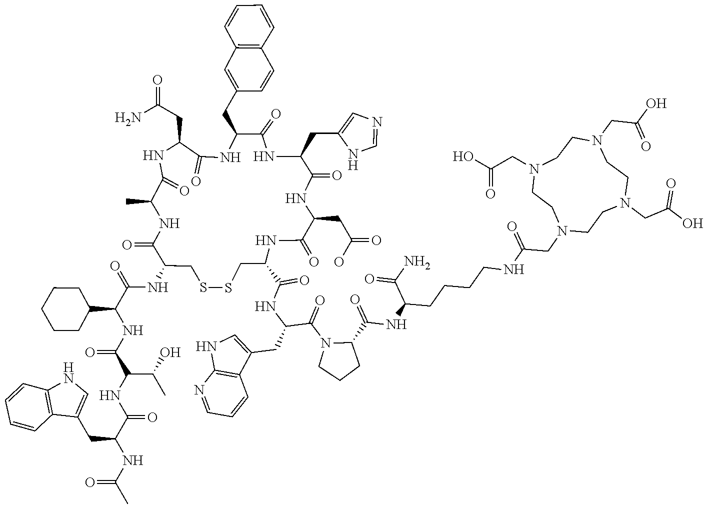 | 194 | 191 |
| C₉₈H₁₃₁N₂₅O₂₅S₂ | | |
| 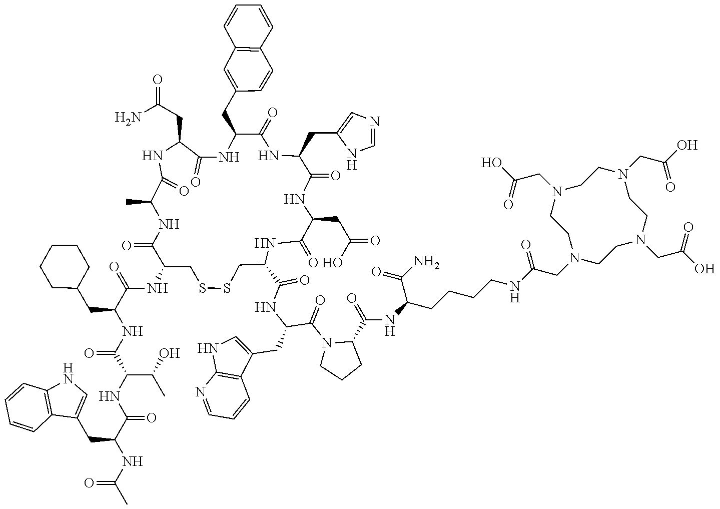 [SMILES: CC(=O)N[C@@H](Cc1c[nH]c2ccccc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](C)NC1=O)[C@@H](C)O] | 195 | 192 |
| C₉₉H₁₃₃N₂₅O₂₅S₂ | | |
| 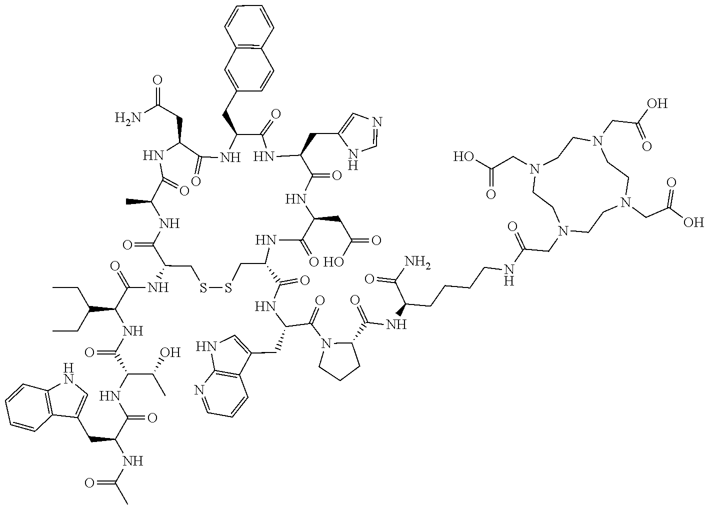 [SMILES: CCC(CC)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](C)NC1=O] | 196 | 193 |
| C₉₇H₁₃₁N₂₅O₂₅S₂ | | |
| 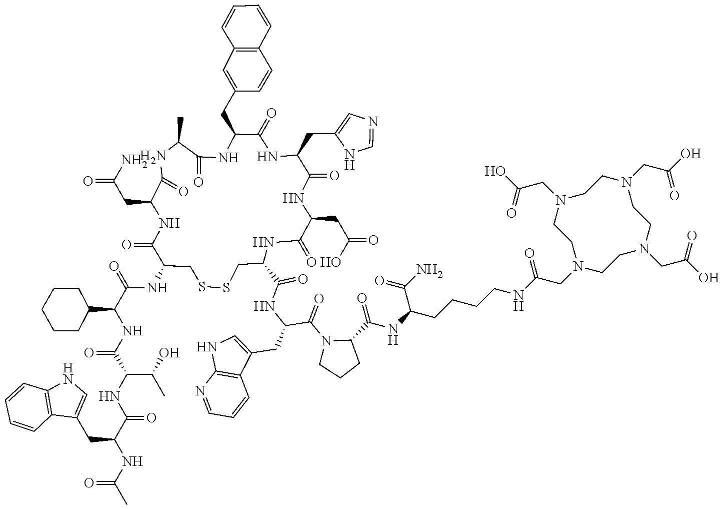 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](C)NC(=O)[C@H](CC(N)=O)NC1=O)C1CCCCC1)[C@@H](C)O] | 197 | 94 |
| C₉₈H₁₃₁N₂₅O₂₅S₂ | | |
| 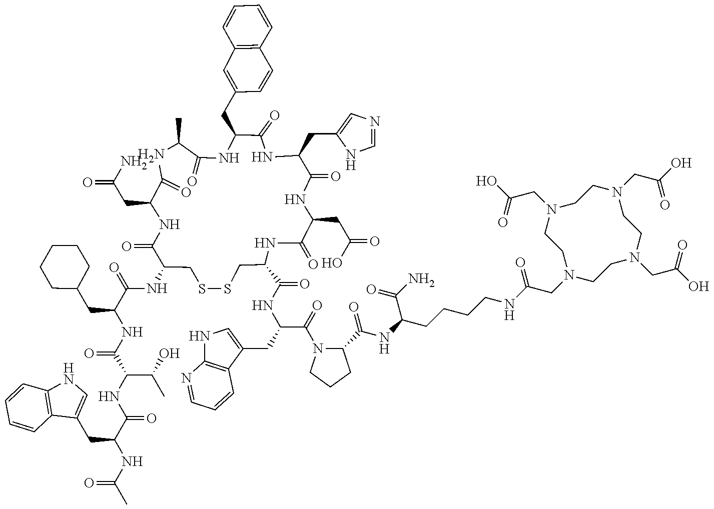 [SMILES: CC(=O)N[C@@H](Cc1c[nH]c2ccccc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](C)NC(=O)[C@H](CC(N)=O)NC1=O)[C@@H](C)O] | 198 | 195 |
| C₉₉H₁₃₃N₂₅O₂₅S₂ | | |
| 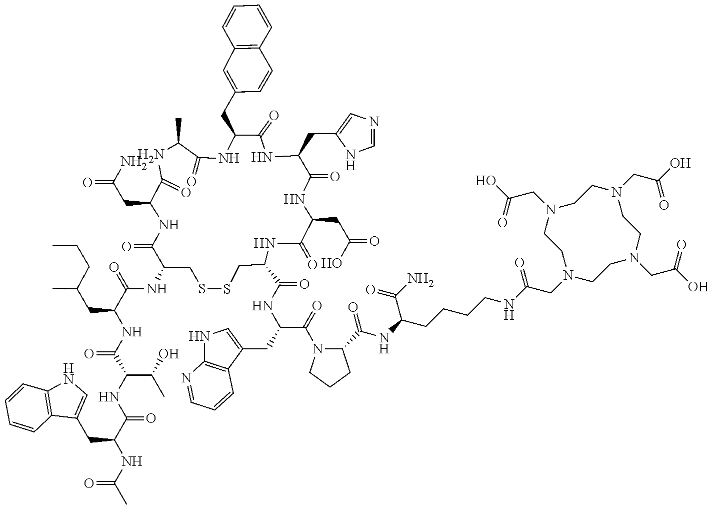 [SMILES: CCCC(C)C[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](C)NC(=O)[C@H](CC(N)=O)NC1=O] | 199 | 196 |
| C₉₇H₁₃₁N₂₅O₂₅S₂ | | |
| 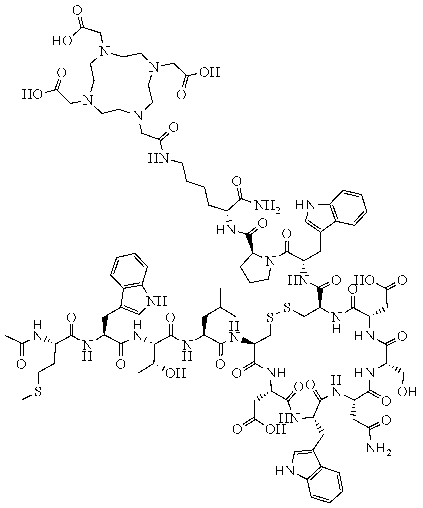 [SMILES: CSCC[C@H](NC(C)=O)C(=O)N[C@@H](Cc1c[nH]c2ccccc12)C(=O)N[C@H](C(=O)N[C@@H](CC(C)C)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](CC(N)=O)NC(=O)[C@H](Cc2c[nH]c3ccccc23)NC(=O)[C@H](CC(=O)O)NC1=O)[C@@H](C)O] | 201 | 197 |
| C₉₈H₁₃₆N₂₄O₂₉S₃ | | |
| 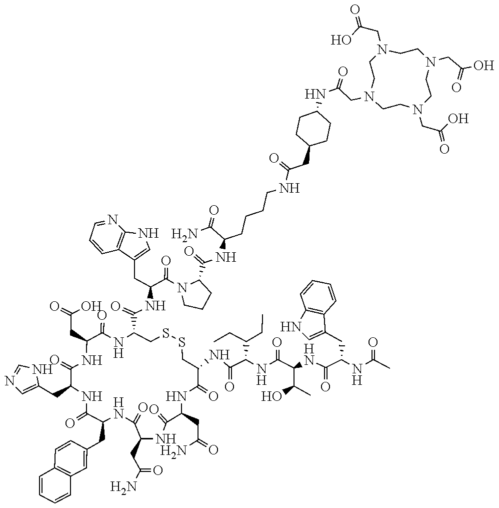 [SMILES: CCC(CC)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)C[C@H]2CC[C@H](NC(=O)CN3CCN(CC(=O)O)CCN(CC(=O)O)CCN(CC(=O)O)CC3)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 202 | 198 |
| C₁₀₆H₁₄₅N₂₇O₂₇S₂ | | |
| 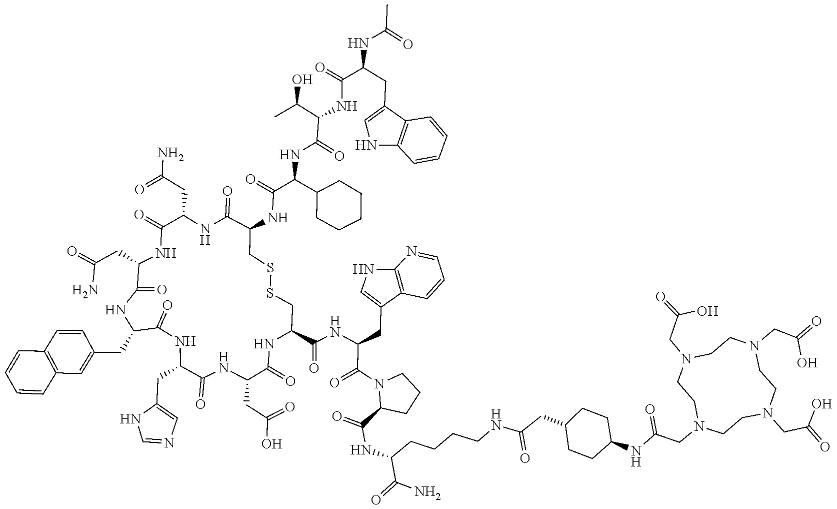 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ncccc23)C(=O)N2CCC[C@H]2C(=O)N[C@H](CCCCNC(=O)C[C@H]2CC[C@H](NC(=O)CN3CCN(CC(=O)O)CCN(CC(=O)O)CCN(CC(=O)O)CC3)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCCCC1)[C@@H](C)O] | 203 | 199 |
| C₁₀₇H₁₄₅N₂₇O₂₇S₂ | | |
| 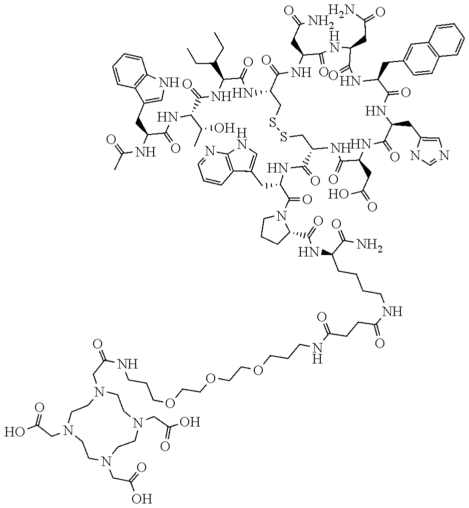 | 204 | 200 |
| C₁₁₂H₁₅₈N₂₈O₃₁S₂ | | |
| 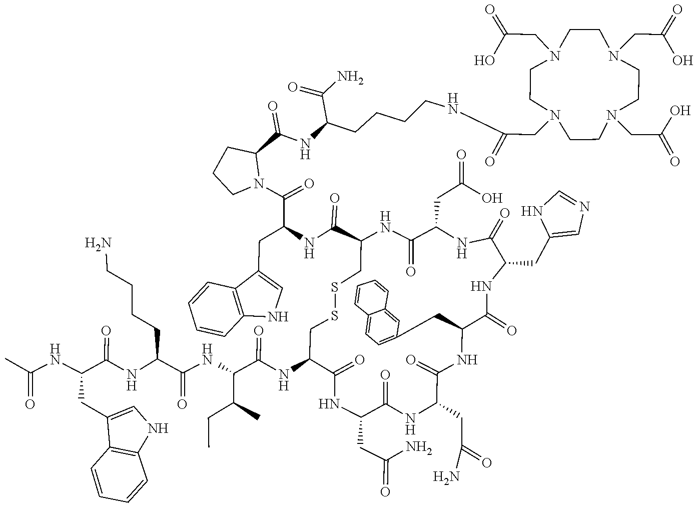 | 205 | 201 |
| C₁₀₀H₁₃₆N₂₆O₂₅S₂ | | |
| 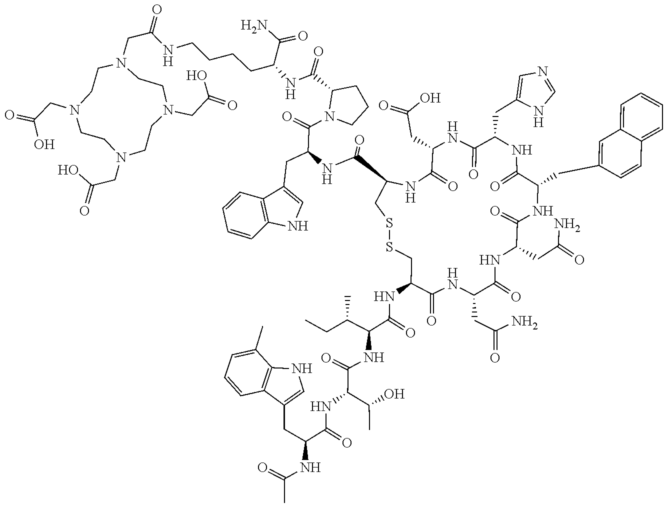 [SMILES: CC[C@H](C)[C@H](NC(=O)[C@@H](NC(=O)[C@H](Cc1c[nH]c2c(C)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 206 | 202 |
| C₉₉H₁₃₃N₂₅O₂₆S₂ | | |
| 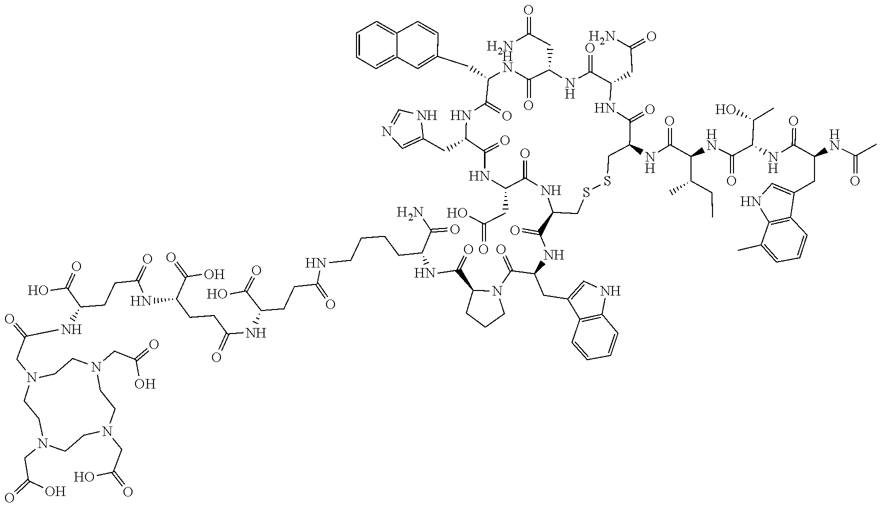 [SMILES: CC[C@H](C)[C@H](NC(=O)[C@@H](NC(=O)[C@H](Cc1c[nH]c2c(C)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 207 | 203 |
| C₁₁₄H₁₅₄N₂₈O₃₅S₂ | | |
| 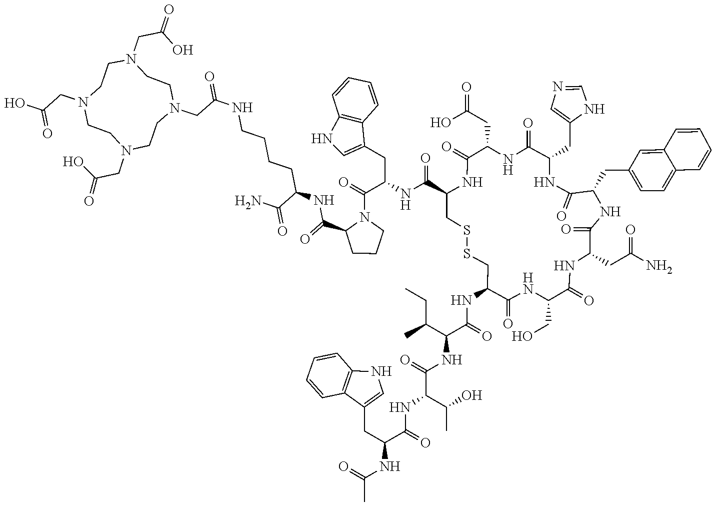 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CO)NC1=O] | 208 | 204 |
| C₉₇H₁₃₀N₂₄O₂₈S₂ | | |
| 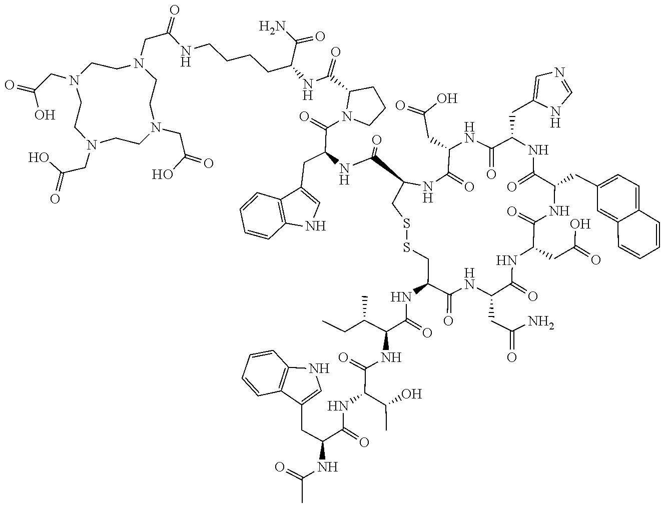 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(=O)O)NC(=O)[C@H](CC(N)=O)NC1=O] | 209 | 205 |
| C₉₈H₁₃₀N₂₄O₂₇S₂ | | |
| 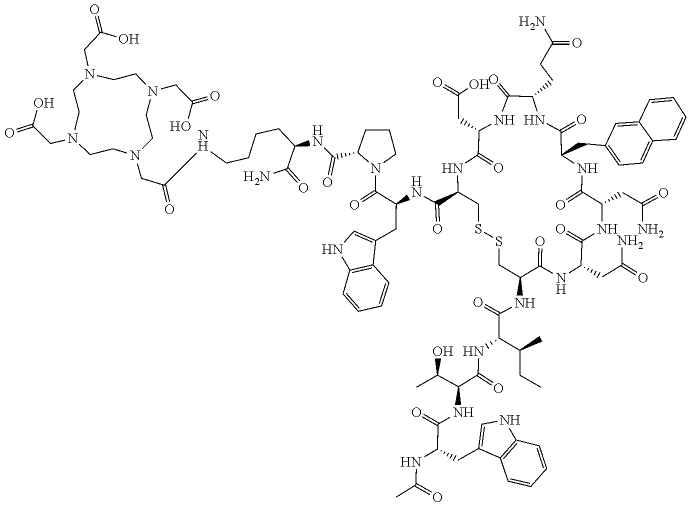 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(N)=O)NC(=O)[C@H](Cc2ccc3ccccc3c2)NC(=O)[C@H](CC(N)=O)NC(=O)[C@H](CC(N)=O)NC1=O] | 210 | 206 |
| C₉₇H₁₃₂N₂₄O₂₇S₂ | | |
| 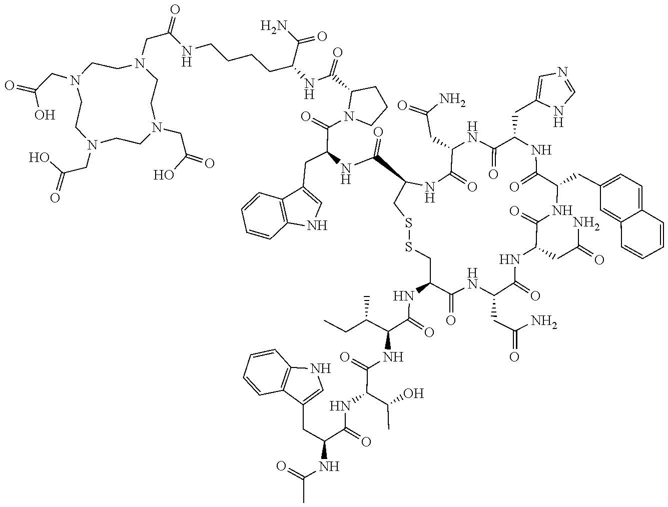 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(N)=O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 211 | 207 |
| C₉₈H₁₃₂N₂₆O₂₅S₂ | | |
| 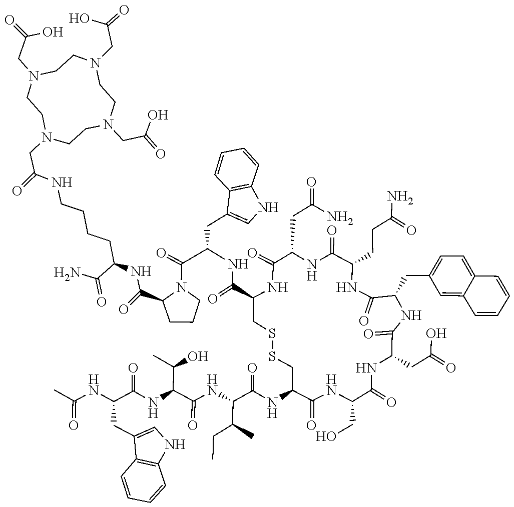 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(N)=O)NC(=O)[C@H](CCC(N)=O)NC(=O)[C@H](Cc2ccc3ccccc3c2)NC(=O)[C@H](CC(=O)O)NC(=O)[C@H](CO)NC1=O] | 212 | 208 |
| C₉₆H₁₃₁N₂₃O₂₇S₂ | | |
| 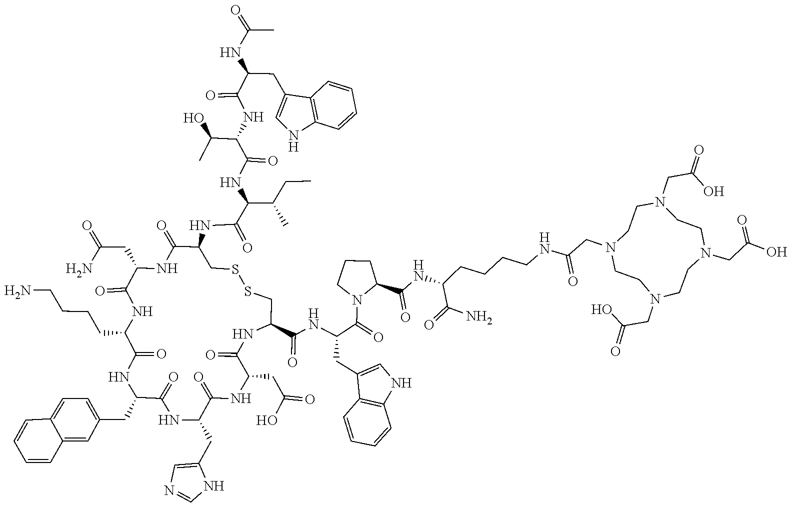 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CCCCN)NC(=O)[C@H](CC(N)=O)NC1=O] | 213 | 209 |
| C₁₀₀H₁₃₇N₂₅O₂₅S₂ | | |
| 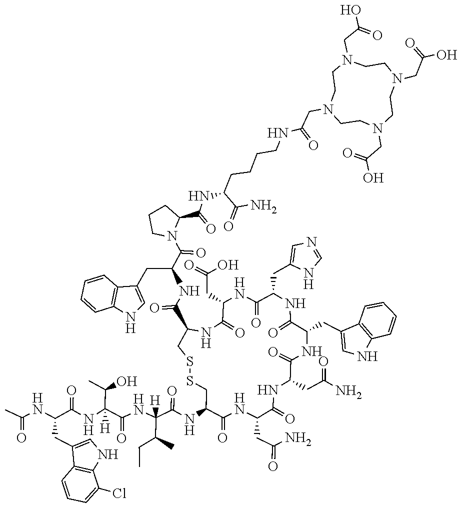 [SMILES: CC[C@H](C)[C@H](NC(=O)[C@@H](NC(=O)[C@H](Cc1c[nH]c2c(Cl)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 226 | 210 |
| C₉₆H₁₂₉ClN₂₆O₂₆S₂ | | |
| 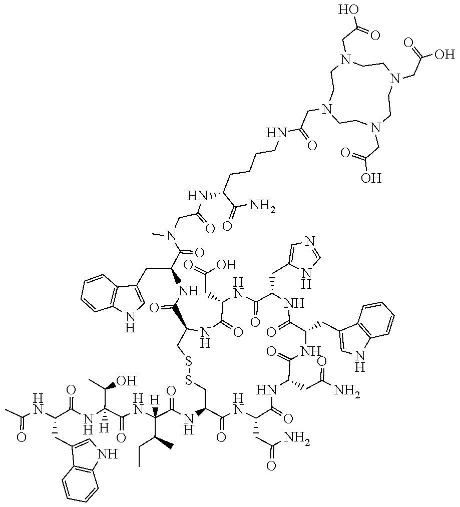 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)CC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 227 | 211 |
| C₉₄H₁₂₈N₂₆O₂₆S₂ | | |
| 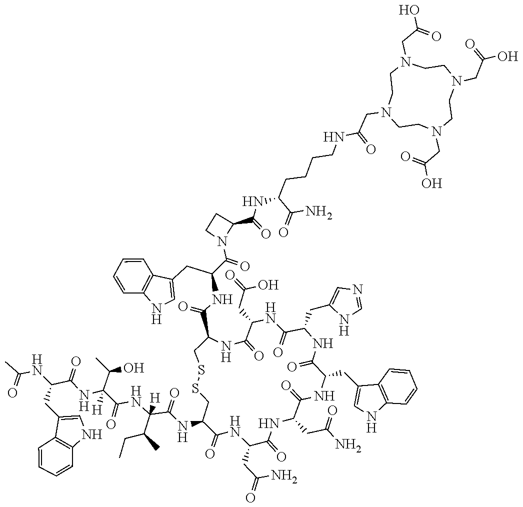 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 228 | 212 |
| C₉₅H₁₂₈N₂₆O₂₆S₂ | | |
| 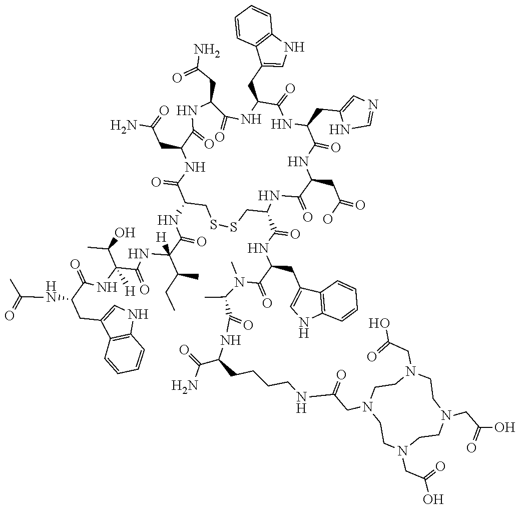 | 229 | 213 |
| C₉₅H₁₃₀N₂₆O₂₆S₂ | | |
| 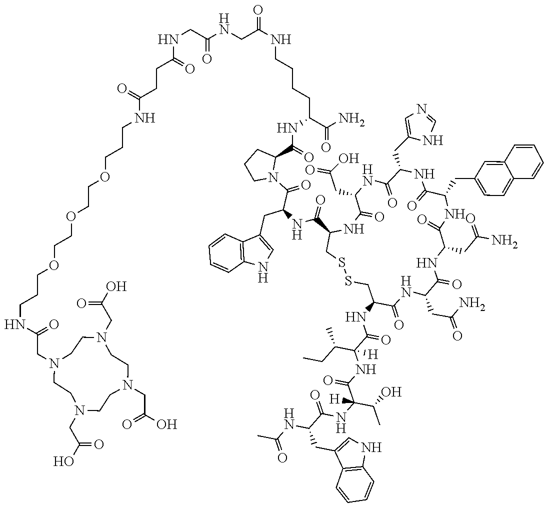 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CNC(=O)CNC(=O)CCC(=O)NCCCOCCOCCOCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 230 | 214 |
| C₁₁₆H₁₆₃N₂₉O₃₃S₂ | | |
| 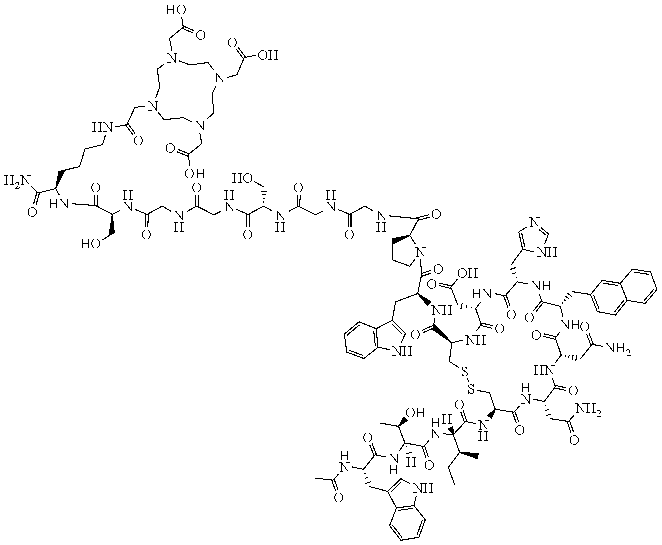 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCC(=O)NCC(=O)N[C@@H](CO)C(=O)NCC(=O)NCC(=O)N[C@@H](CO)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 231 | 215 |
| C₁₁₂H₁₅₃N₃₁O₃₄S₂ | | |
| 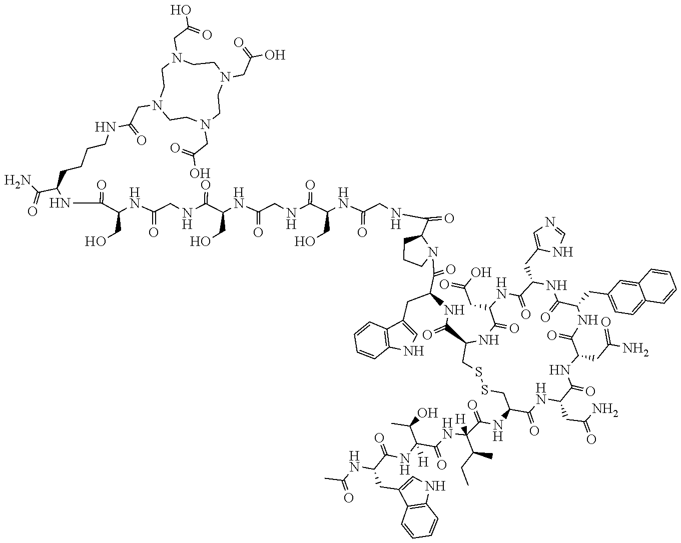 | 232 | 216 |
| C₁₁₃H₁₅₅N₃₁O₃₅S₂ | | |
| 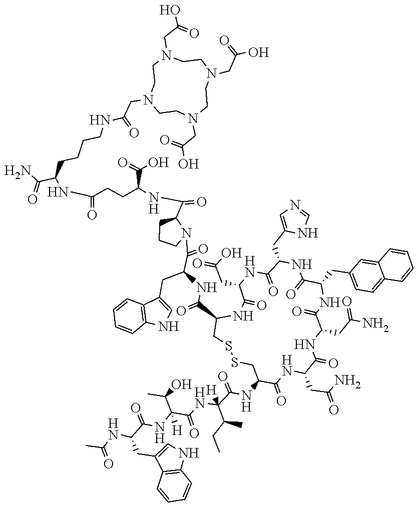 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@@H](CCC(=O)N[C@H](CCCCNC(=O)CN2CCN(CC(=O)O)CCN(CC(=O)O)CCN(CC(=O)O)CC2)C(N)=O)C(=O)O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 233 | 217 |
| C₁₀₃H₁₃₈N₂₆O₂₉S₂ | | |
| 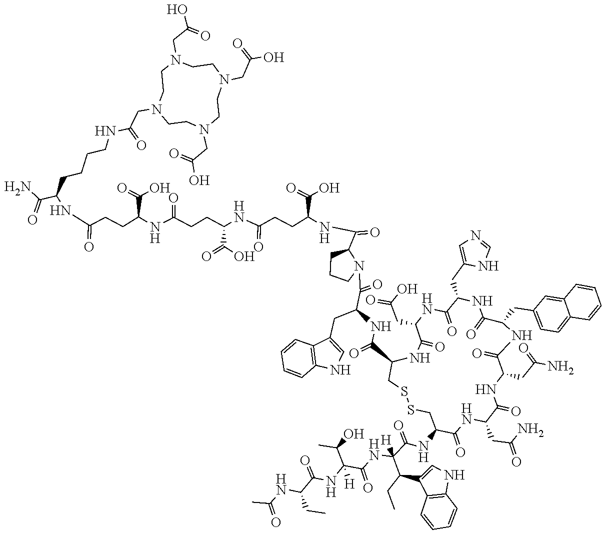 [SMILES: CC[C@H](NC(C)=O)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@@H](CCC(=O)N[C@@H](CCC(=O)N[C@@H](CCC(=O)N[C@H](CCCCNC(=O)CN2CCN(CC(=O)O)CCN(CC(=O)O)CCN(CC(=O)O)CC2)C(N)=O)C(=O)O)C(=O)O)C(=O)O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@H](CC)c1c[nH]c2ccccc12)[C@@H](C)O] | 234 | 218 |
| C₁₁₃H₁₅₂N₂₆O₃₅S₂ | | |
| 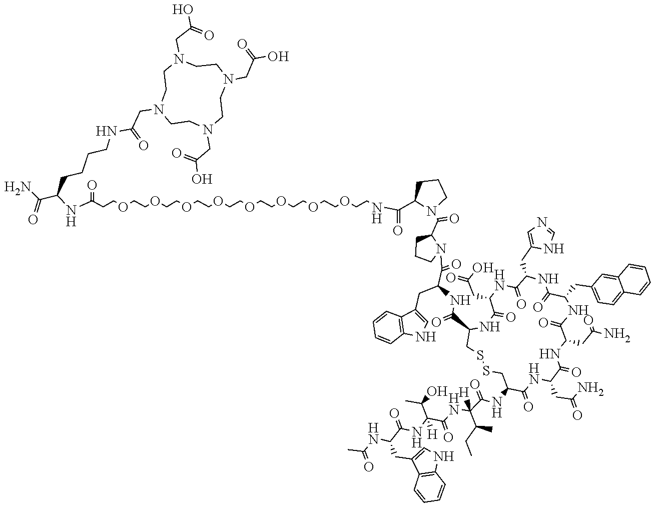 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N2CCC[C@@H]2C(=O)NCCOCCOCCOCCOCCOCCOCCOCCOCCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 235 | 219 |
| C₁₂₂H₁₇₅N₂₇O₃₆S₂ | | |
| 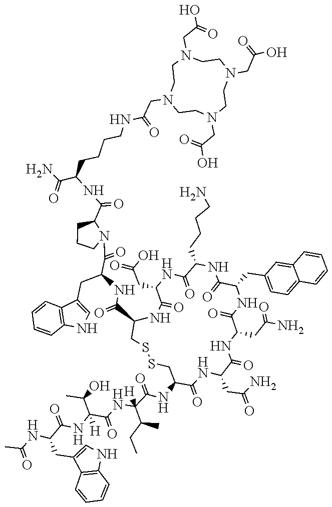 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCCCN)NC(=O)[C@H](Cc2ccc3ccccc3c2)NC(=O)[C@H](CC(N)=O)NC(=O)[C@H](CC(N)=O)NC1=O] | 237 | 220 |
| C₉₈H₁₃₆N₂₄O₂₆S₂ | | |
| 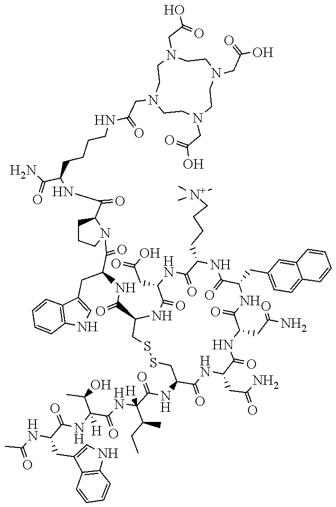 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCCC[N+](C)(C)C)NC(=O)[C@H](Cc2ccc3ccccc3c2)NC(=O)[C@H](CC(N)=O)NC(=O)[C@H](CC(N)=O)NC1=O] | 238 | 221 |
| C₁₀₁H₁₄₃N₂₄O₂₆S₂⁺ | | |
| 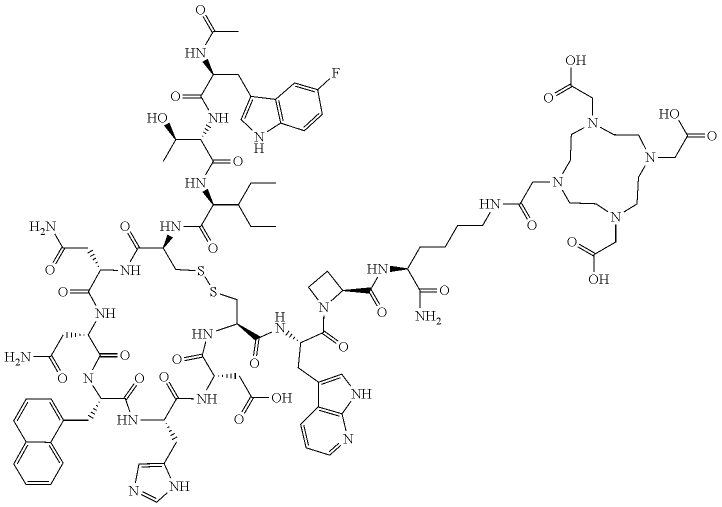 | 241 | 222 |
| C₉₇H₁₂₉FN₂₆O₂₆S₂ | | |
| 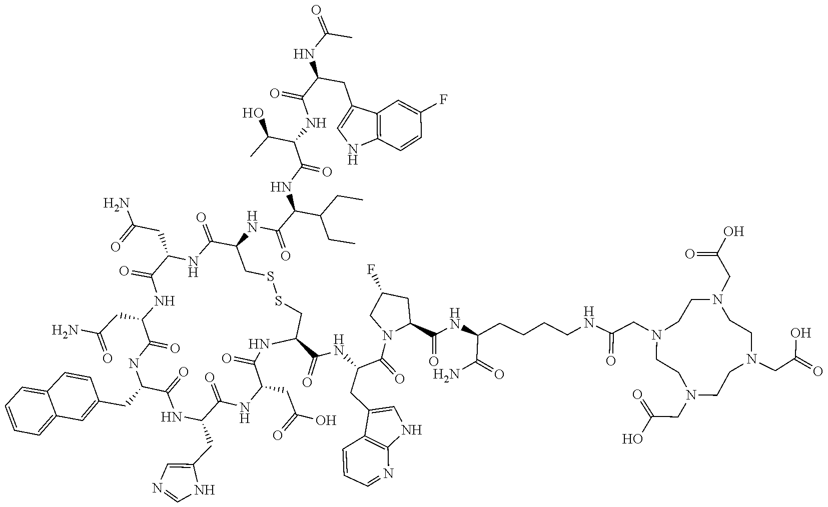 | 242 | 223 |
| C₉₈H₁₃₀F₂N₂₆O₂₆S₂ | | |
| 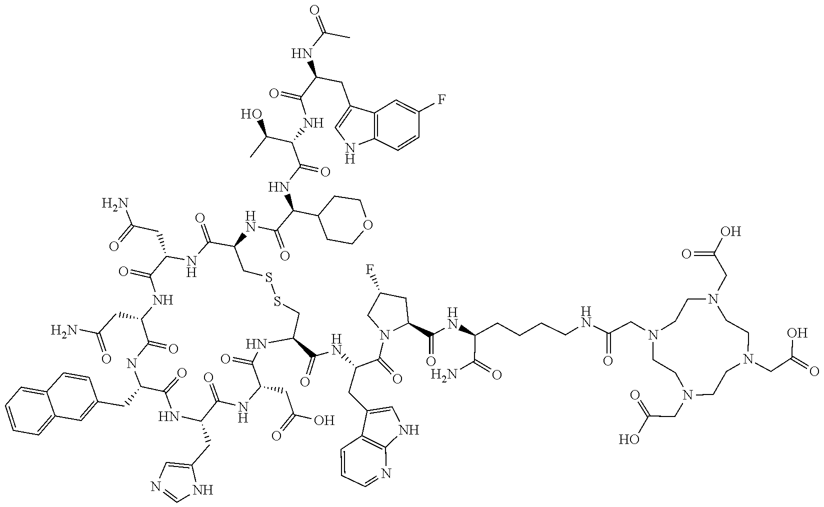 | 243 | 224 |
| C₉₈H₁₂₈F₂N₂₆O₂₇S₂ | | |
| 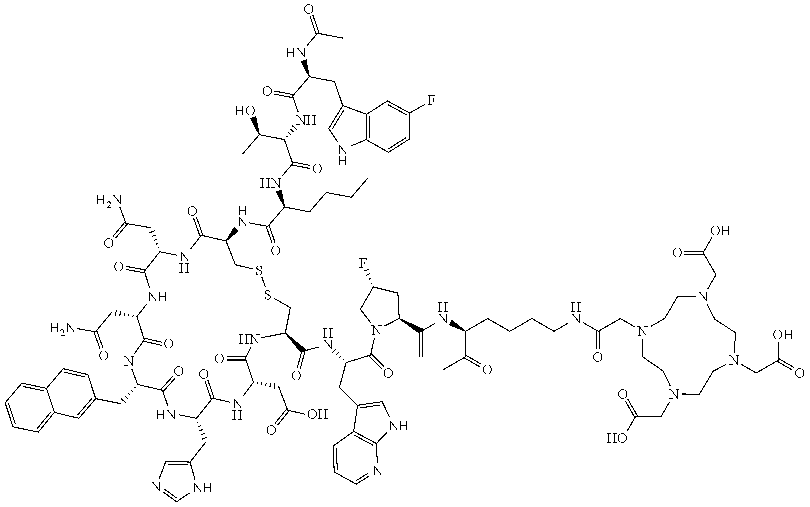 | 244 | 225 |
| C₉₇H₁₂₈F₂N₂₆O₂₀S₂ | | |
| 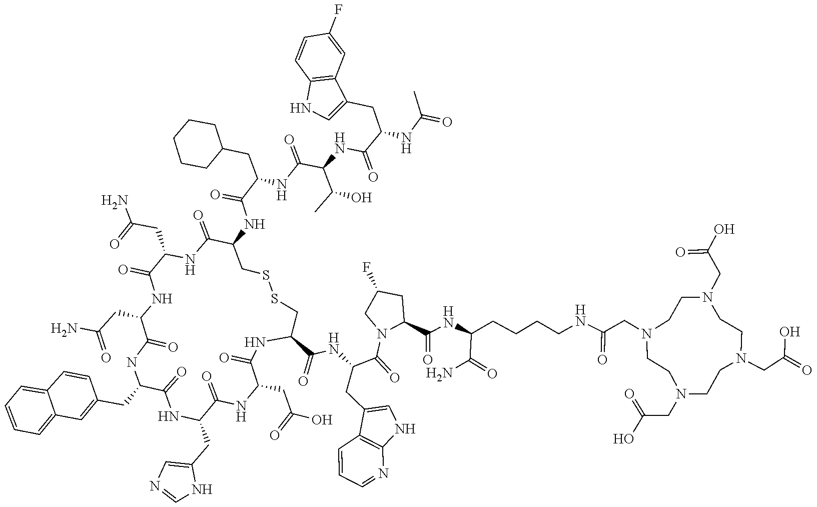 | 245 | 226 |
| C₁₀₀H₁₃₂F₂N₂₆O₂₆S₂ | | |
| 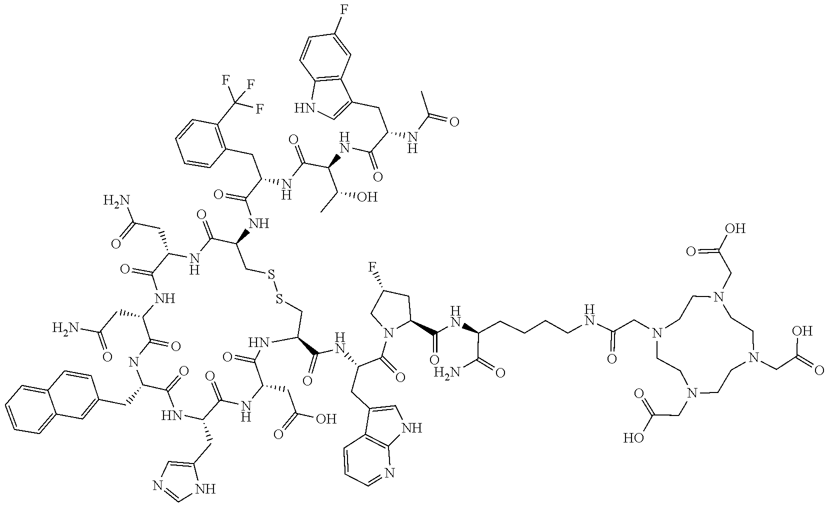 | 246 | 227 |
| C₁₀₁H₁₂₅F₅N₂₆O₂₆S₂ | | |
| 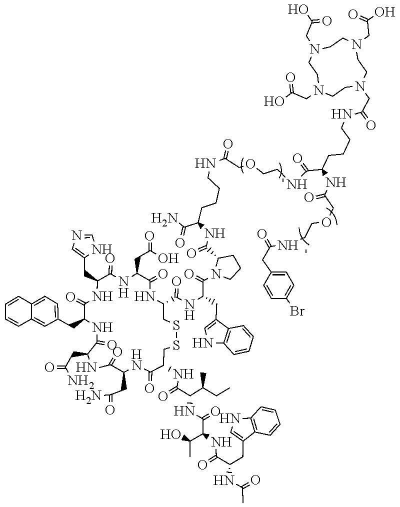 | 251 | 228 |
| C₁₄₈H₂₁₈BrN₂₉O₄₆S₂ | | |
| 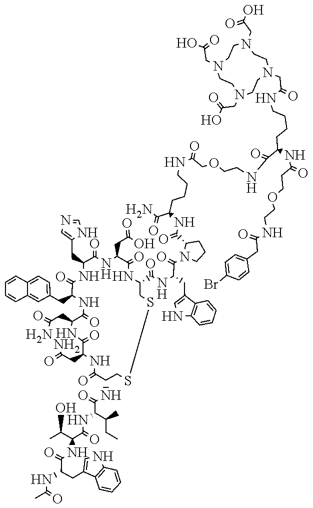 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)COCCNC(=O)[C@@H](CCCCNC(=O)CN2CCN(CC(=O)O)CCN(CC(=O)O)CCN(CC(=O)O)CC2)NC(=O)CCOCCNC(=O)Cc2ccc(Br)cc2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 252 | 229 |
| C₁₅₇H₂₃₆BrN₂₉O₅₀S₂ | | |
| 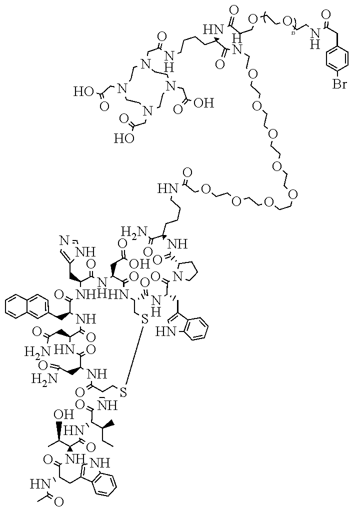 | 255 | 230 |
| C₁₈₁H₂₈₄BrN₂₉O₆₂S₂ | | |
| 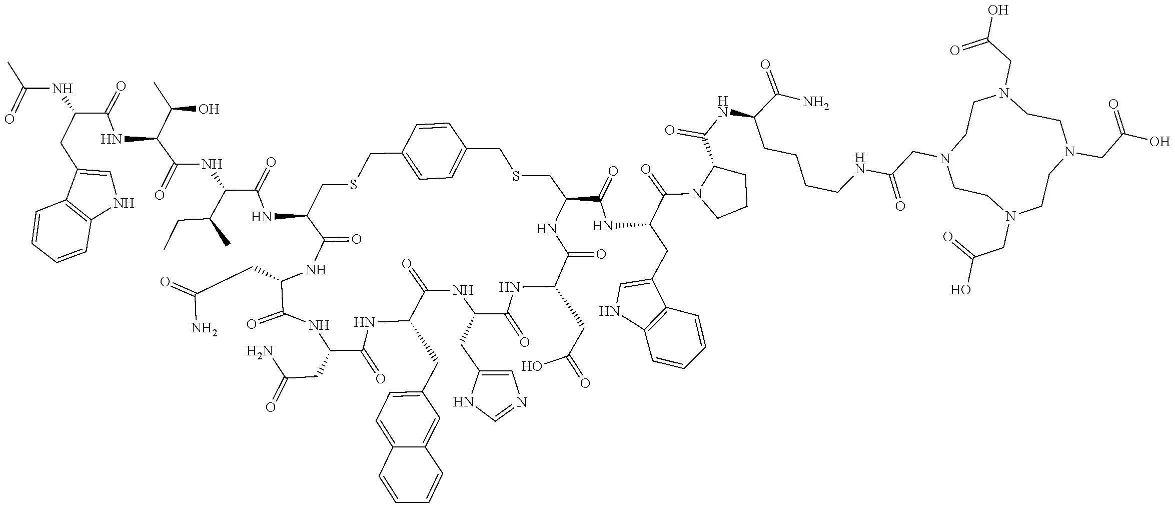 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCc2ccc(cc2)CSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](C(C)C(N)=O)NC(=O)[C@H](CC(N)=O)NC1=O] | 256 | 231 |
| C₁₀₆H₁₃₉N₂₅O₂₆S₂ | | |
| 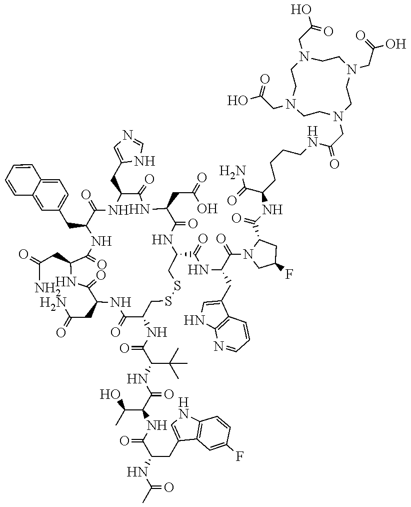 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C(C)(C)C)[C@@H](C)O] | 257 | 232 |
| C₉₇H₁₂₈F₂N₂₆O₂₆S₂ | | |
| 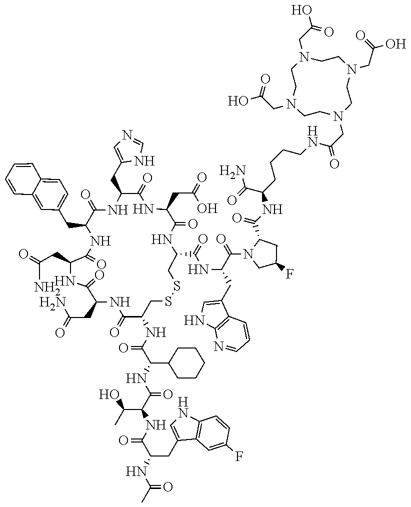 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCCCC1)[C@@H](C)O] | 258 | 233 |
| C₉₉H₁₃₀F₂N₂₆O₂₆S₂ | | |
| 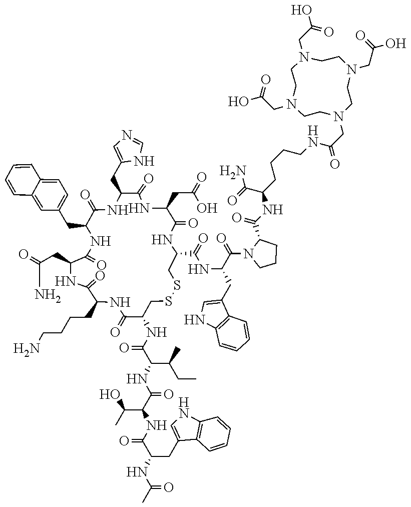 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CCCCN)NC1=O] | 259 | |
| C₁₀₀H₁₃₇N₂₅O₂₅S₂ | | |
| 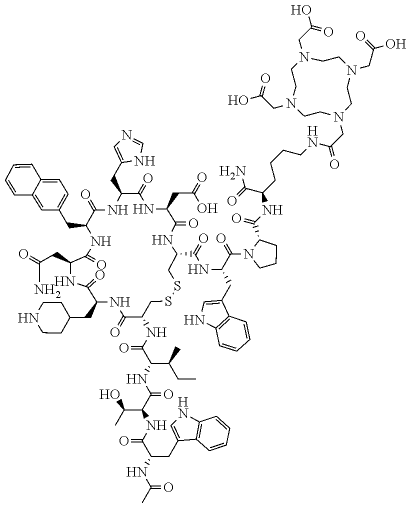 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O] | 260 | 235 |
| C₁₀₂H₁₃₉N₂₅O₂₅S₂ | | |
| 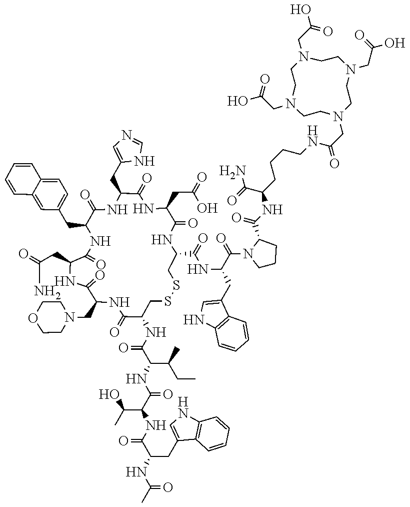 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CN2CCOCC2)NC1=O] | 261 | 235 |
| C₁₀₁H₁₃₇N₂₅O₂₆S₂ | | |
| 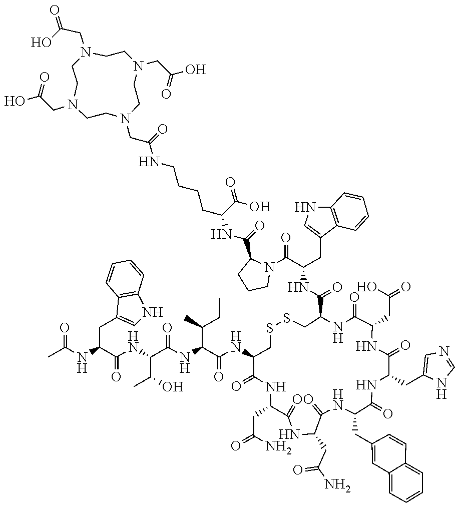 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(=O)O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 262 | 236 |
| C₉₈H₁₃₀N₂₄O₂₇S₂ | | |
| 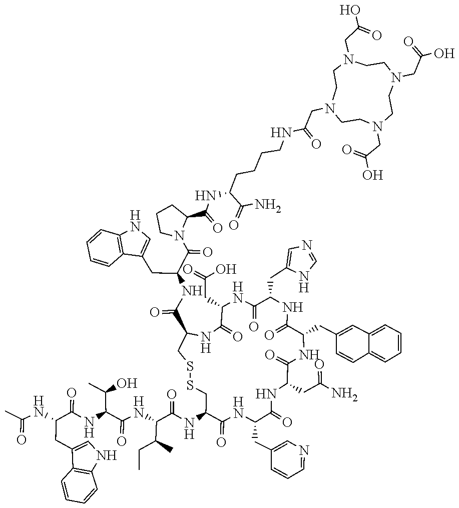 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O] | 263 | 237 |
| C₁₀₂H₁₃₃N₂₅O₂₅S₂ | | |
| 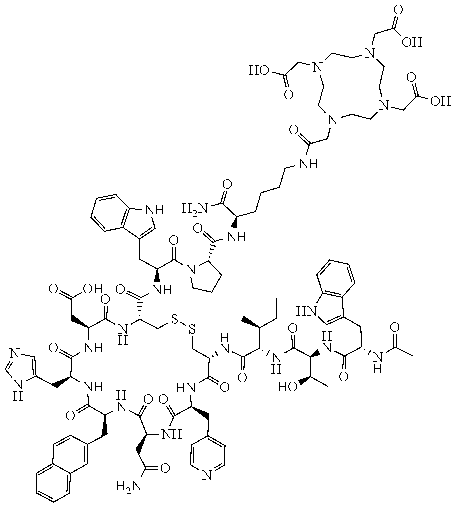 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2ccncc2)NC1=O] | 264 | 238 |
| C₁₀₂H₁₃₃N₂₅O₂₅S₂ | | |
| 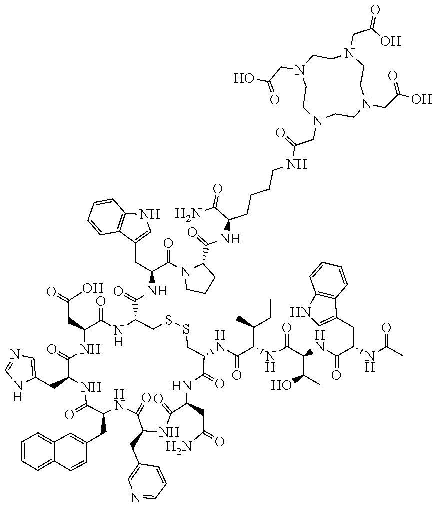 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](Cc2cccnc2)NC(=O)[C@H](CC(N)=O)NC1=O] | 265 | 239 |
| C₁₀₂H₁₃₃N₂₅O₂₅S₂ | | |
| 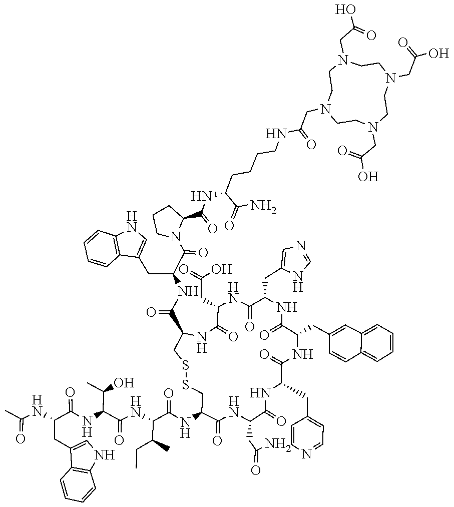 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](Cc2ccncc2)NC(=O)[C@H](CC(N)=O)NC1=O] | 266 | 240 |
| C₁₀₂H₁₃₃N₂₅O₂₅S₂ | | |
| 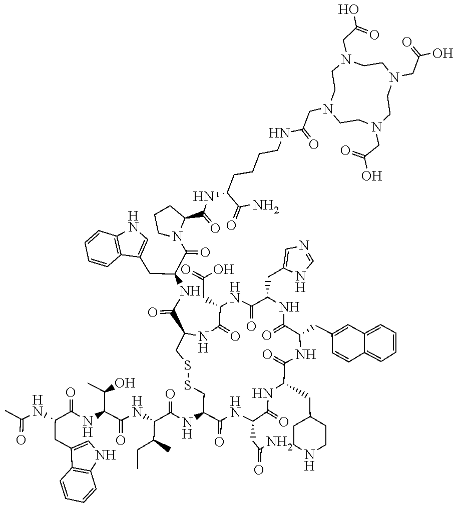 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC2CCNCC2)NC(=O)[C@H](CC(N)=O)NC1=O] | 267 | 241 |
| C₁₀₂H₁₃₉N₂₅O₂₅S₂ | | |
| 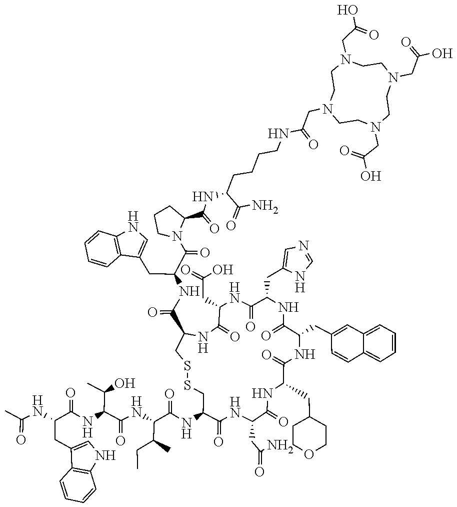 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC2CCOCC2)NC(=O)[C@H](CC(N)=O)NC1=O] | 268 | 242 |
| C₁₀₁H₁₃₇N₂₅O₂₆S₂ | | |
| 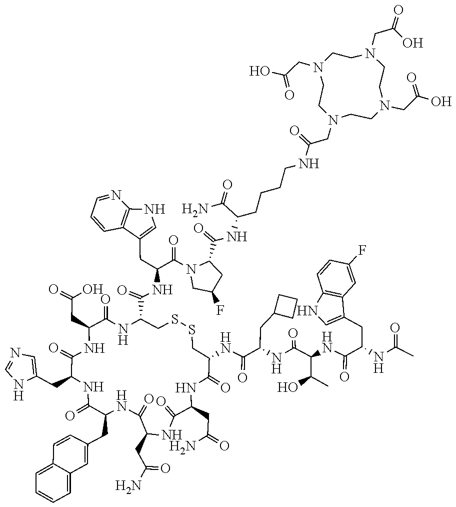 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O] | 269 | 243 |
| C₉₈H₁₂₈F₂N₂₆O₂₆S₂ | | |
| 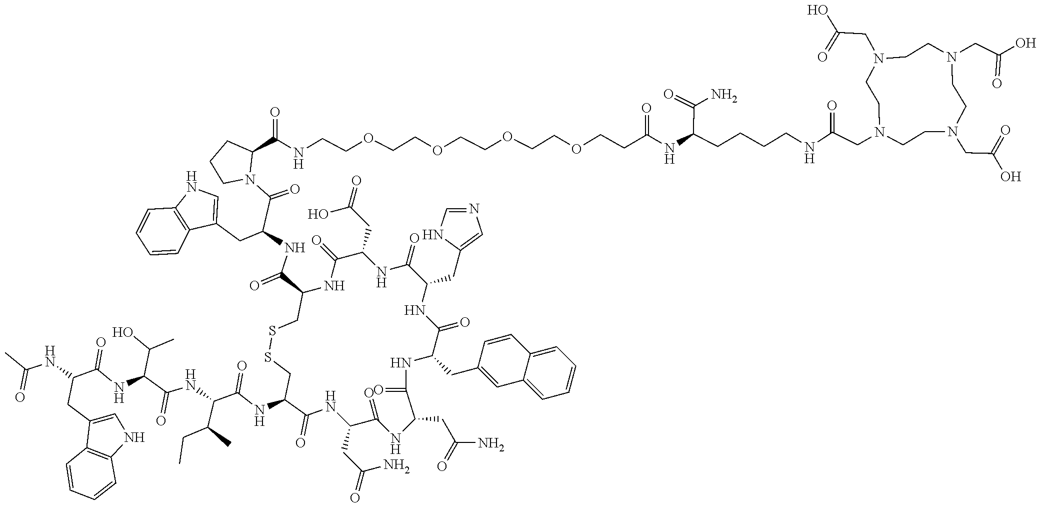 | 270 | 244 |
| C₁₀₉H₁₅₂₃N₂₆O₃₁S₂ | | |
| 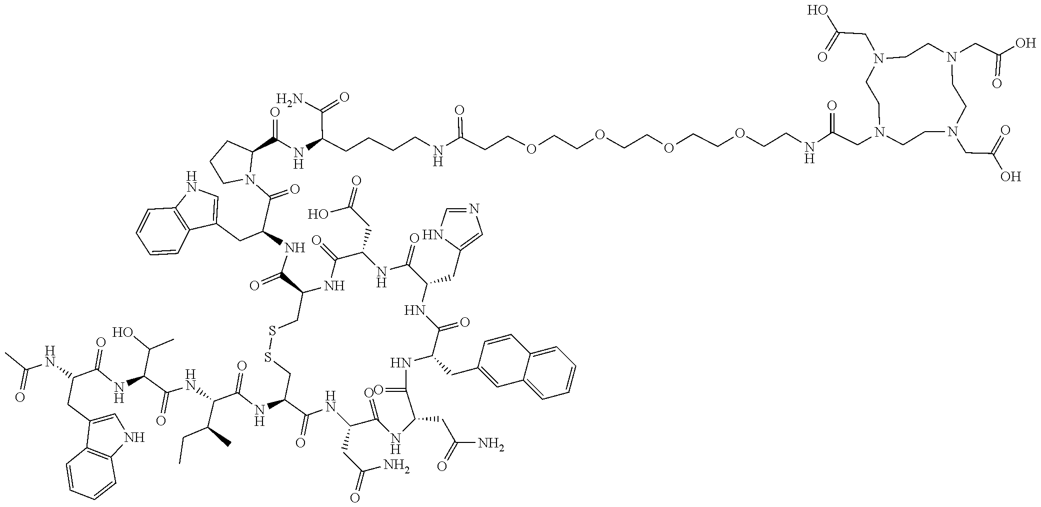 | 271 | 245 |
| C₁₀₀H₁₅₂N₂₆O₃₁S₂ | | |
| 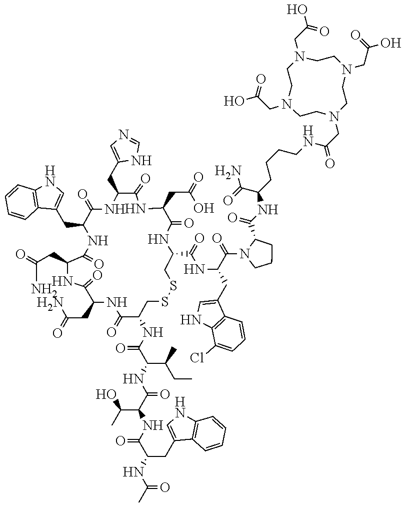 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3c(Cl)cccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 272 | 246 |
| C₉₆H₁₂₉ClN₂₆O₂₆S₂ | | |
| 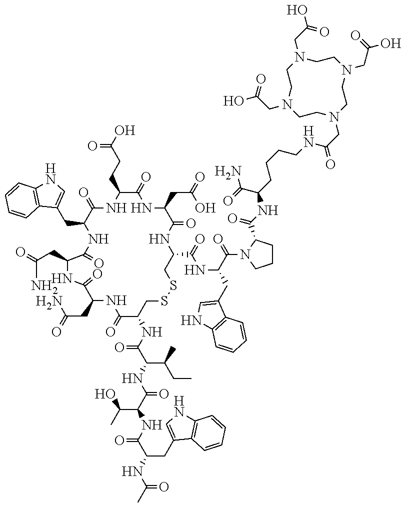 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CC(N)=O)NC(=O)[C@H](CC(N)=O)NC1=O] | 273 | 247 |
| C₉₆H₁₃₁N₂₃O₂₉S₂ | | |
| 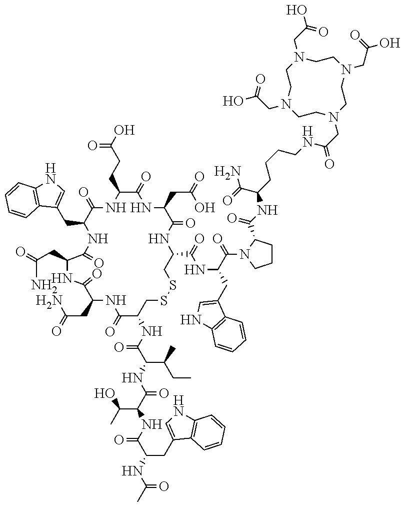 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CC(N)=O)NC(=O)[C@H](CC(N)=O)NC1=O] | 274 | 248 |
| C₉₅H₁₃₀N₂₄O₂₈S₂ | | |
| 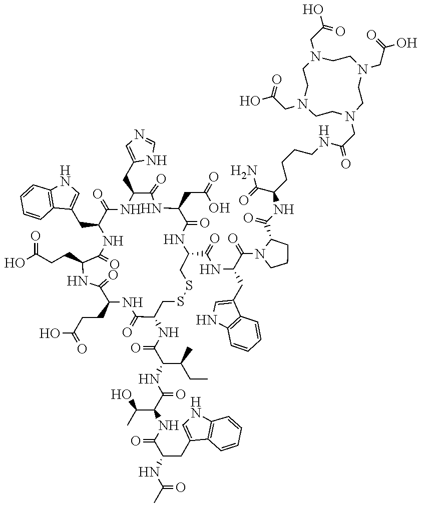 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CCC(=O)O)NC(=O)[C@H](CCC(=O)O)NC1=O] | 275 | 249 |
| C₉₇H₁₃₂N₂₂O₃₀S₂ | | |
| 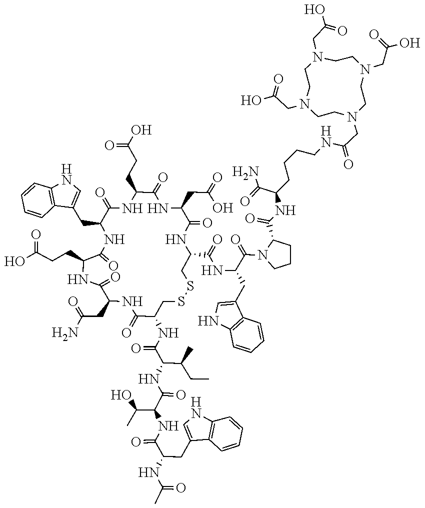 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CCC(=O)O)NC(=O)[C@H](CC(N)=O)NC1=O] | 276 | 250 |
| C₉₆H₁₃₁N₂₃O₂₉S₂ | | |
| 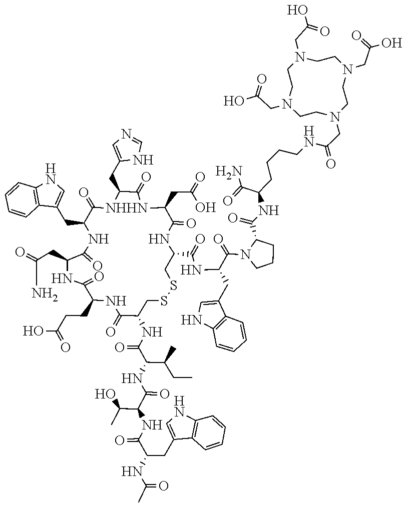 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CCC(=O)O)NC1=O] | 277 | 251 |
| C₉₇H₁₃₁N₂₅O₂₇S₂ | | |
| 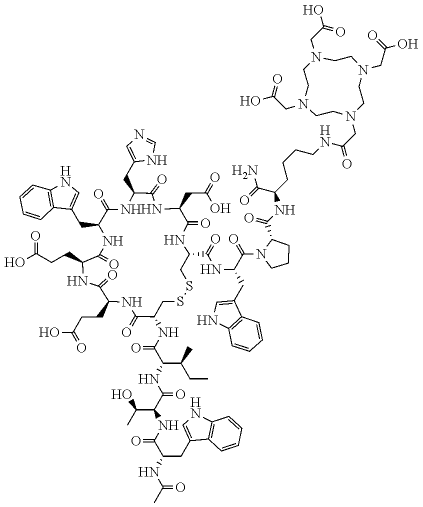 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CCC(=O)O)NC(=O)[C@H](CCC(=O)O)NC1=O] | 278 | 252 |
| C₉₈H₁₃₂N₂₄O₂₈S₂ | | |
| 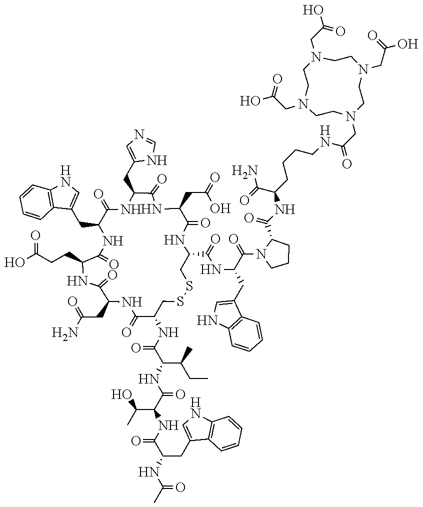 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CCC(=O)O)NC(=O)[C@H](CC(N)=O)NC1=O] | 279 | 253 |
| C₉₇H₁₃₁N₂₅O₂₇S₂ | | |
| 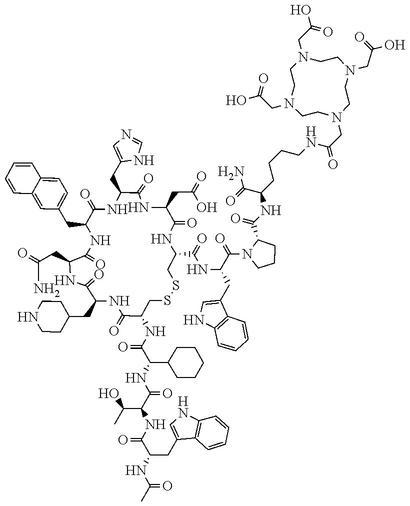 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O] | 280 | 254 |
| C₁₀₄H₁₄₁N₂₅O₂₅S₂ | | |
| 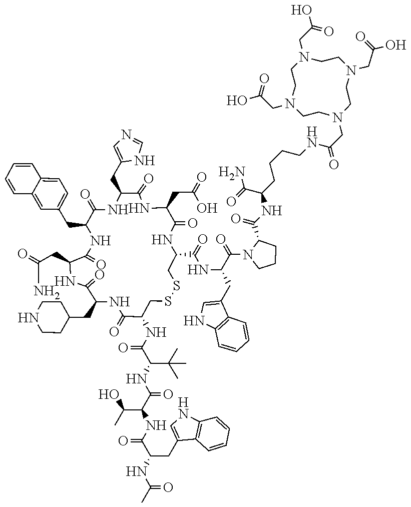 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C(C)(C)C)[C@@H](C)O] | 281 | 255 |
| C₁₀₂H₁₃₉N₂₅O₂₅S₂ | | |
| 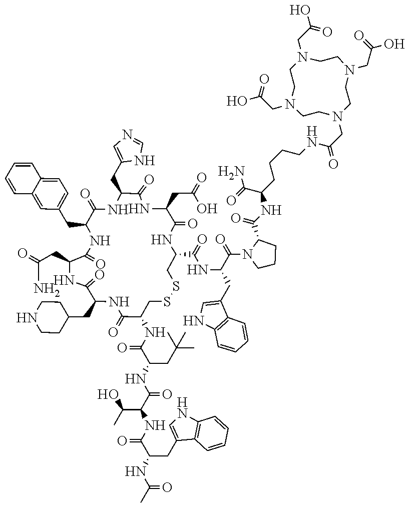 [SMILES: CC(=O)N[C@@H](Cc1c[nH]c2ccccc12)C(=O)N[C@H](C(=O)N[C@@H](CC(C)(C)C)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)[C@@H](C)O] | 282 | 256 |
| C₁₀₃H₁₄₁N₂₅O₂₅S₂ | | |
| 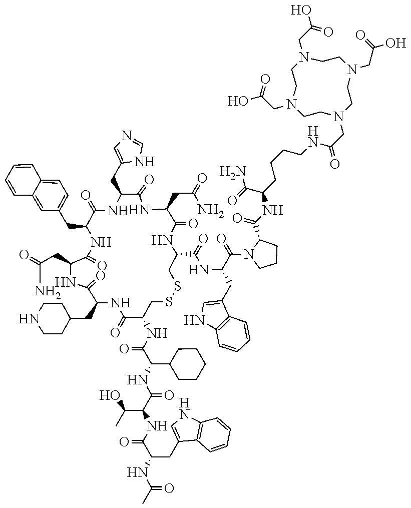 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(N)=O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O] | 283 | 257 |
| C₁₀₄H₁₄₂N₂₆O₂₄S₂ | | |
| 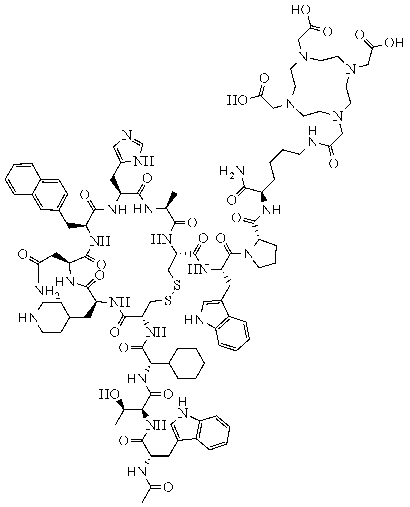 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](C)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O] | 284 | 258 |
| C₁₀₃H₁₄₁N₂₅O₂₃S₂ | | |
| 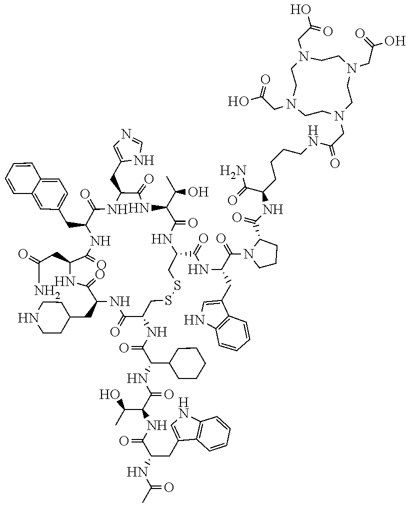 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H]([C@@H](C)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O] | 285 | 259 |
| C₁₀₄H₁₄₃N₂₅O₂₄S₂ | | |
| 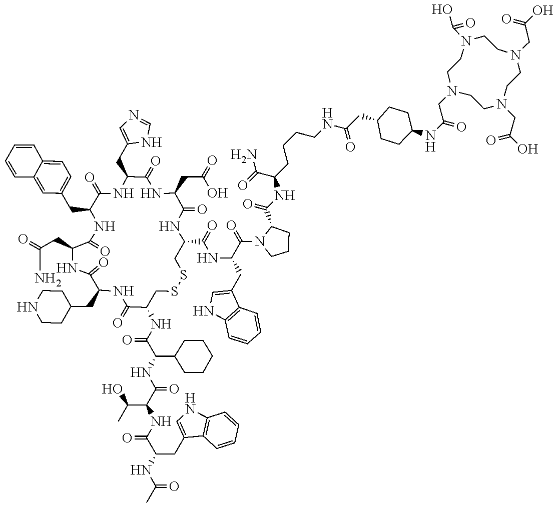 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)C[C@H]2CC[C@H](NC(=O)CN3CCN(CC(=O)O)CCN(CC(=O)O)CCN(C(=O)O)CC3)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O] | 286 | 260 |
| C₁₁₂H₁₅₄N₂₆O₂₆S₂ | | |
| 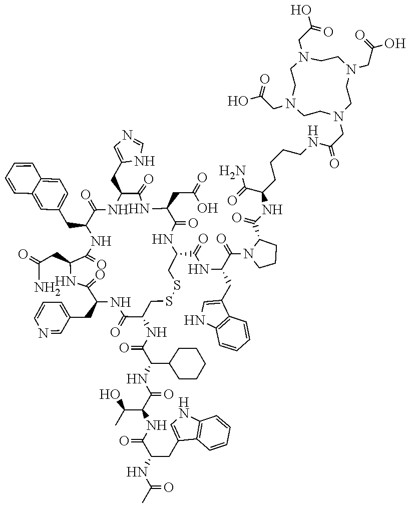 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O)C1CCCCC1)[C@@H](C)O] | 287 | 261 |
| C₁₀₄H₁₃₅N₂₅O₂₅S₂ | | |
| 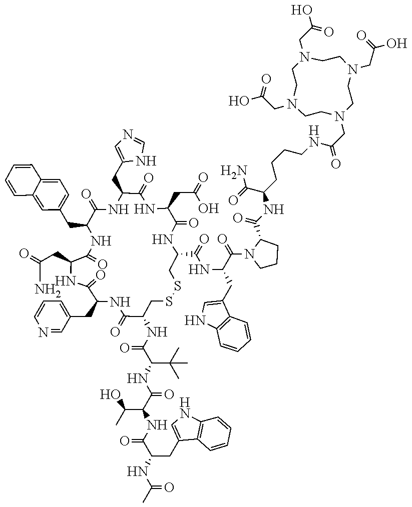 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O)C(C)(C)C)[C@@H](C)O] | 288 | 262 |
| C₁₀₂H₁₃₃N₂₅O₂₅S₂ | | |
| 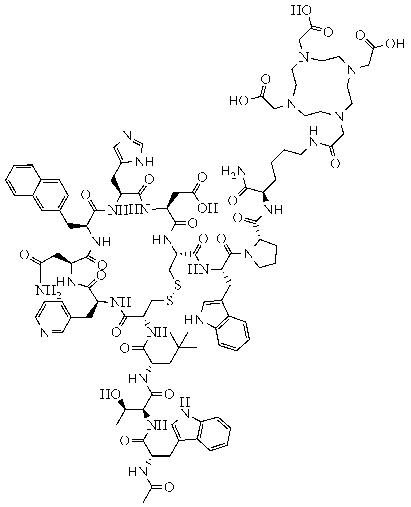 [SMILES: CC(=O)N[C@@H](Cc1c[nH]c2ccccc12)C(=O)N[C@H](C(=O)N[C@@H](CC(C)(C)C)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O)[C@@H](C)O] | 289 | 263 |
| C₁₀₃H₁₃₅N₂₅O₂₅S₂ | | |
| 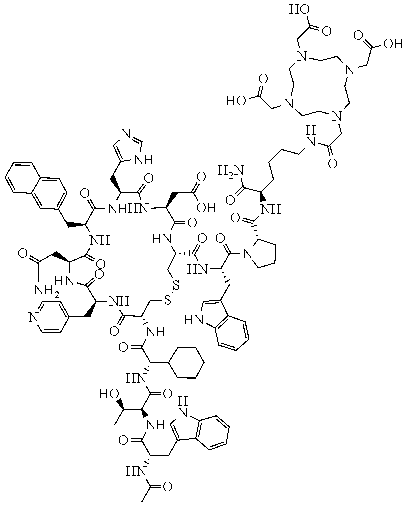 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2ccncc2)NC1=O)C1CCCCC1)[C@@H](C)O] | 290 | 264 |
| C₁₀₄H₁₃₅N₂₅O₂₅S₂ | | |
| 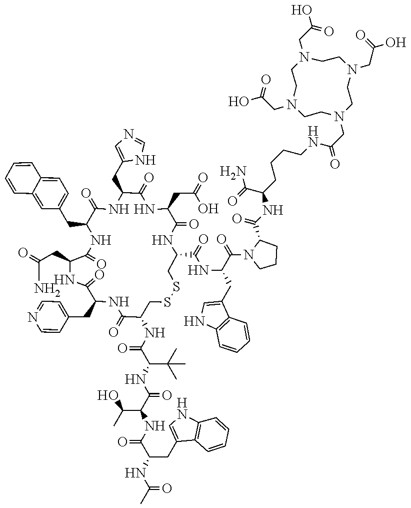 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2ccncc2)NC1=O)C(C)(C)C)[C@@H](C)O] | 291 | 265 |
| C₁₀₂H₁₃₃N₂₅O₂₅S₂ | | |
| 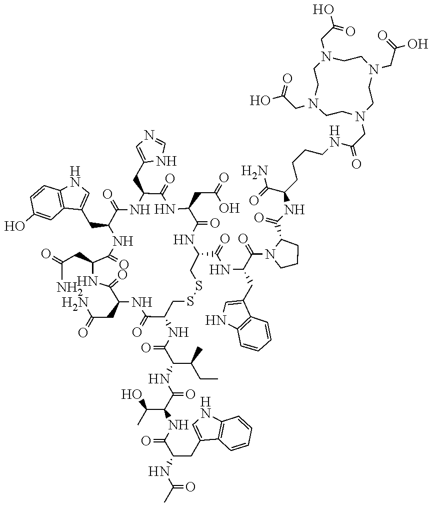 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccc(O)cc23)NC(=O)[C@H](CC(N)=O)NC(=O)[C@H](CC(N)=O)NC1=O] | 292 | 266 |
| C₉₆H₁₃₀N₂₆O₂₇S₂ | | |
| 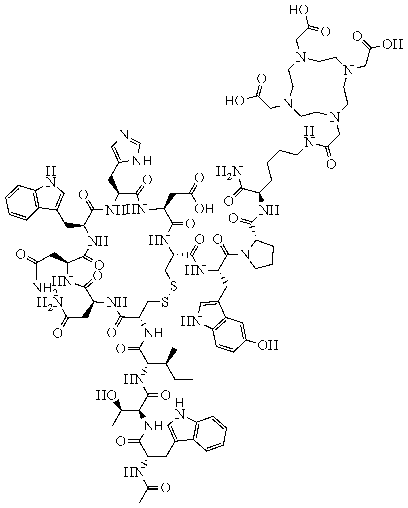 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccc(O)cc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 293 | 267 |
| C₉₆H₁₃₀N₂₆O₂₇S₂ | | |
| 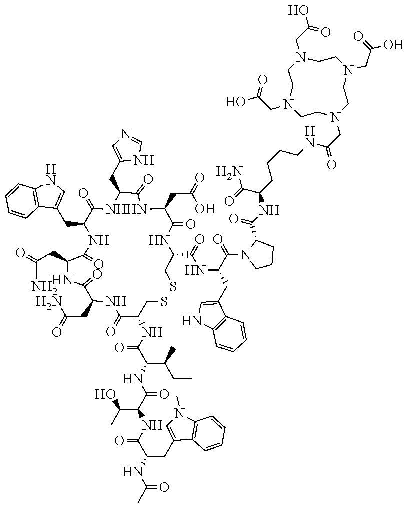 [SMILES: CC[C@H](C)[C@H](NC(=O)[C@@H](NC(=O)[C@H](Cc1cn(C)c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 294 | 268 |
| C₉₇H₁₃₂N₂₆O₂₆S₂ | | |
| 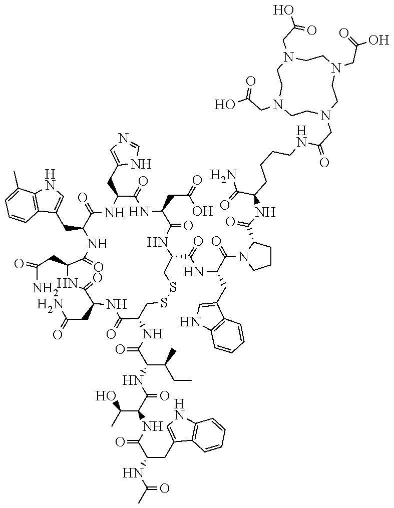 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3c(C)cccc23)NC(=O)[C@H](CC(N)=O)NC(=O)[C@H](CC(N)=O)NC1=O] | 295 | 269 |
| C₉₇H₁₃₂N₂₆O₂₆S₂ | | |
| 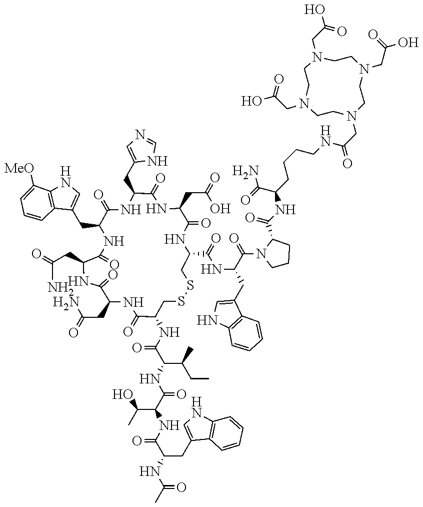 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3c(OC)cccc23)NC(=O)[C@H](CC(N)=O)NC(=O)[C@H](CC(N)=O)NC1=O] | 296 | 270 |
| C₉₇H₁₃₂N₂₆O₂₇S₂ | | |
| 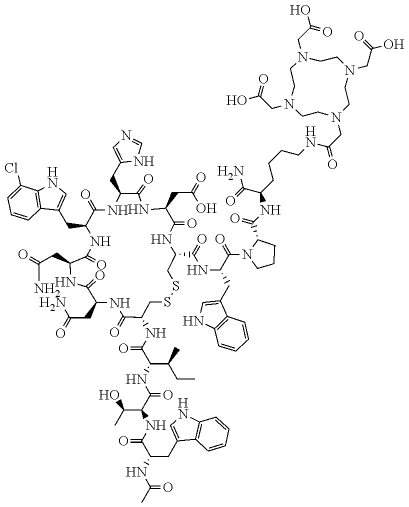 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3c(Cl)cccc23)NC(=O)[C@H](CC(N)=O)NC(=O)[C@H](CC(N)=O)NC1=O] | 297 | 271 |
| C₉₆H₁₂₉ClN₂₆O₂₆S₂ | | |
| 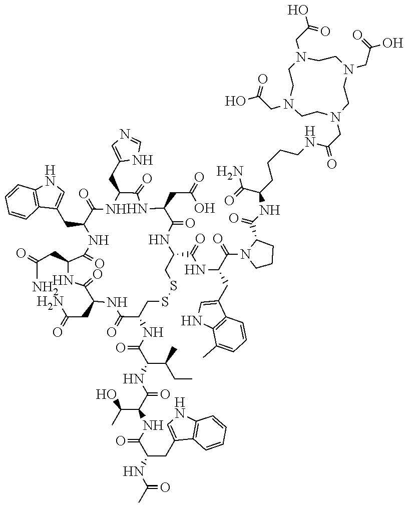 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3c(C)cccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 298 | 272 |
| C₉₇H₁₃₂N₂₆O₂₆S₂ | | |
| 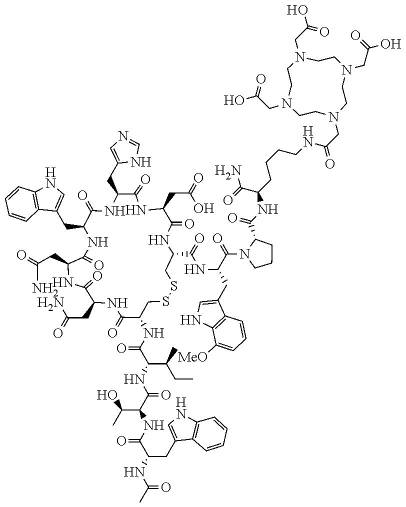 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3c(OC)cccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 299 | 273 |
| C₉₇H₁₃₂N₂₆O₂₇S₂ | | |
| 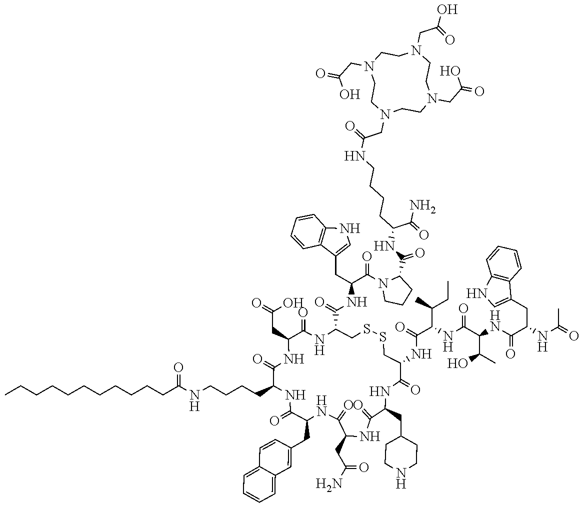 | 326 | 274 |
| C₁₁₄H₁₆₆N₂₄O₂₆S₂ | | |
| 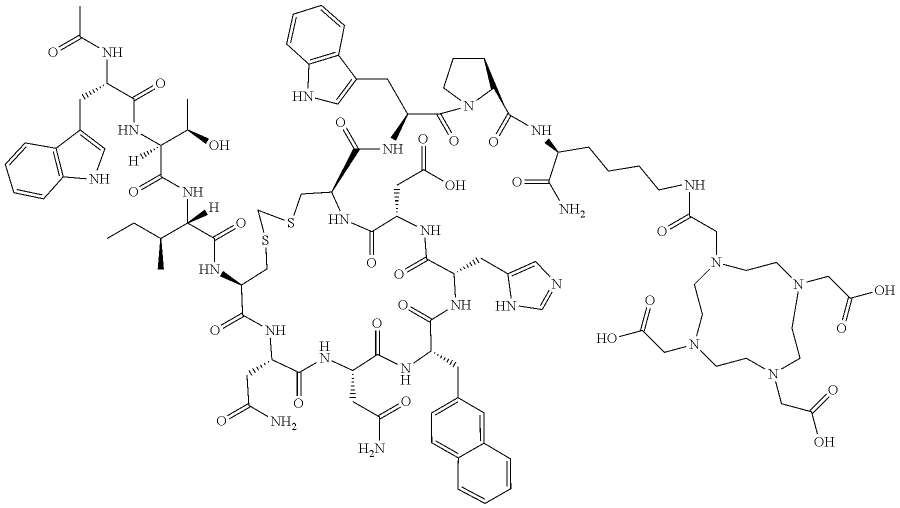 | 329 | 275 |
| C₉₉H₁₃₃N₂₅O₂₆S₂ | | |
| 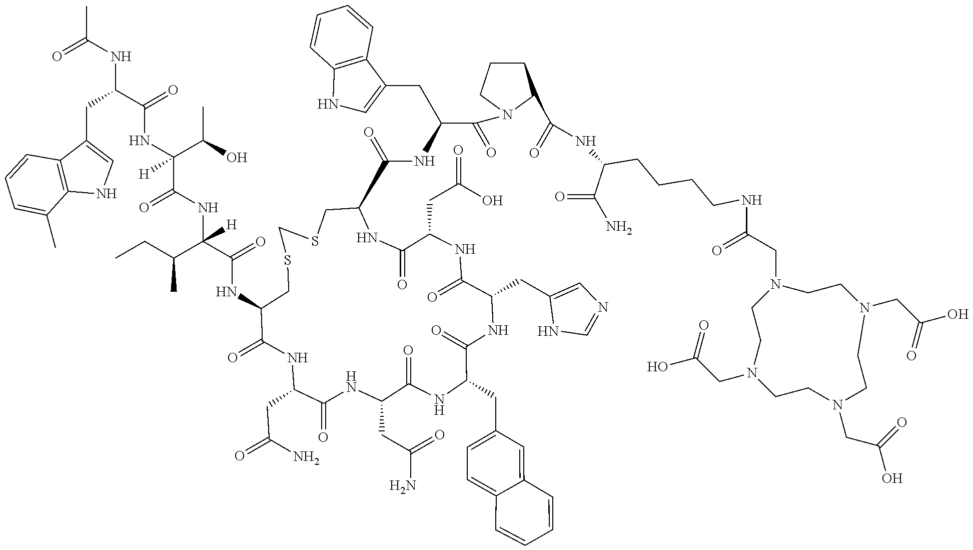 | 330 | 276 |
| C₁₀₀H₁₃₅N₂₅O₂₆S₂ | | |
| 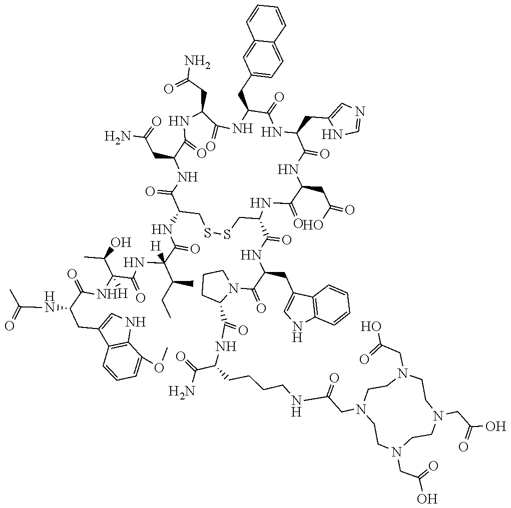 [SMILES: CC[C@H](C)[C@H](NC(=O)[C@@H](NC(=O)[C@H](Cc1c[nH]c2c(OC)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 344 | 277 |
| C₉₉H₁₃₃N₂₅O₂₇S₂ | | |
| 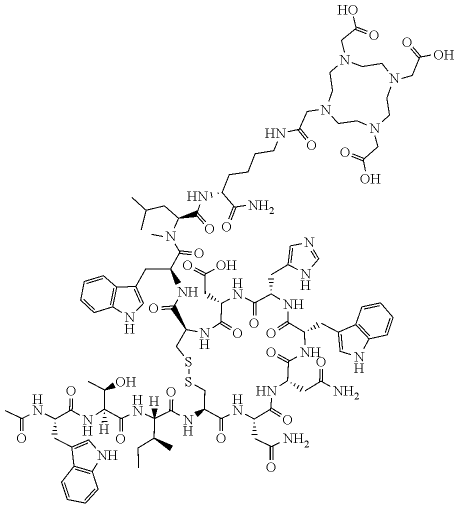 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)[C@@H](CC(C)C)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 345 | 278 |
| C₉₈H₁₃₆N₂₆O₂₆S₂ | | |
| 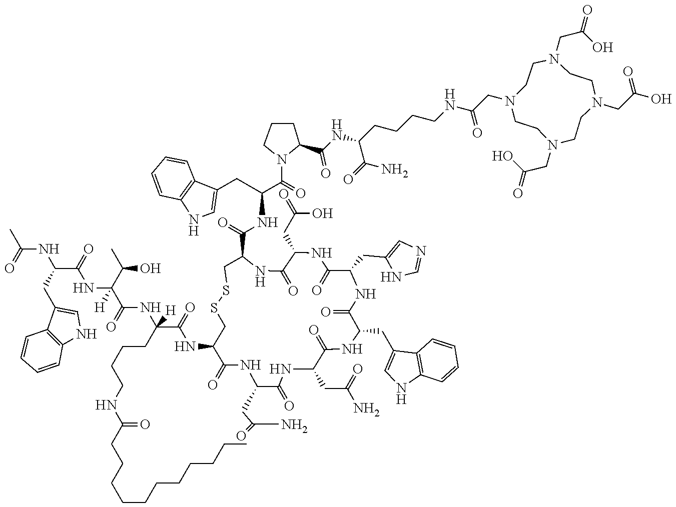 [SMILES: CCCCCCCCCCCC(=O)NCCCC[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 346 | 279 |
| C₁₀₈H₁₅₃N₂₇O₂₇S₂ | | |
| 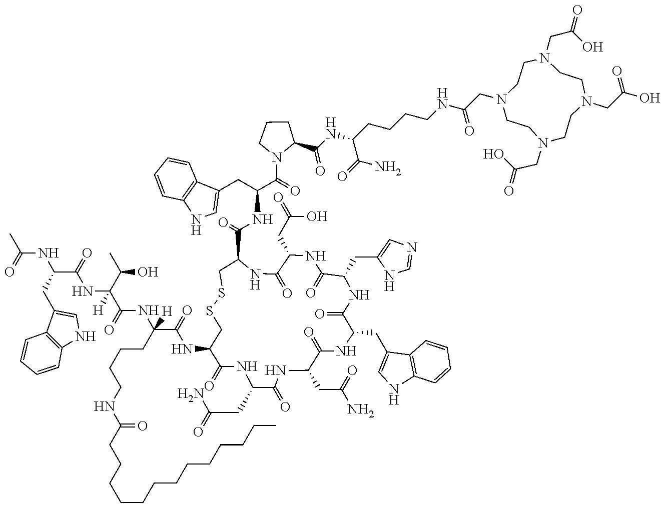 [SMILES: CCCCCCCCCCCCCC(=O)NCCCC[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 347 | 280 |
| C₁₁₀H₁₅₇N₂₇O₂₇S₂ | | |
| 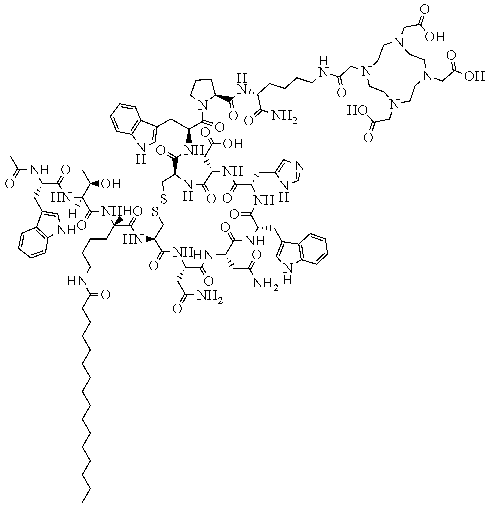 [SMILES: CCCCCCCCCCCCCCCC(=O)NCCCC[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 348 | 281 |
| C₁₁₂H₁₆₁N₂₇O₂₇S₂ | | |
| 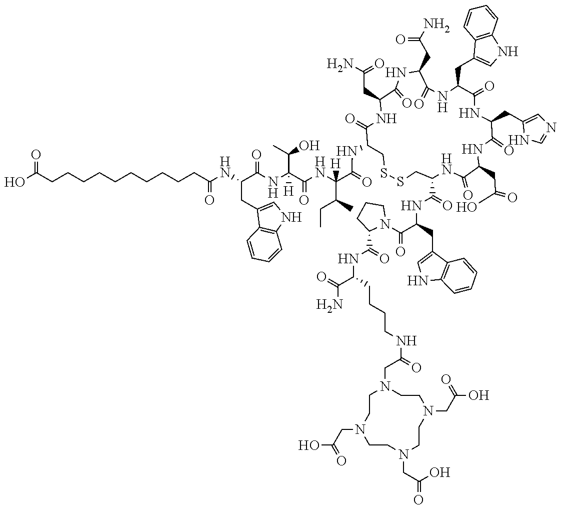 [SMILES: CC[C@H](C)[C@H](NC(=O)[C@@H](NC(=O)[C@H](Cc1c[nH]c2ccccc12)NC(=O)CCCCCCCCCCC(=O)O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 349 | 282 |
| C₁₀₆H₁₄₈N₂₆O₂₈S₂ | | |
| 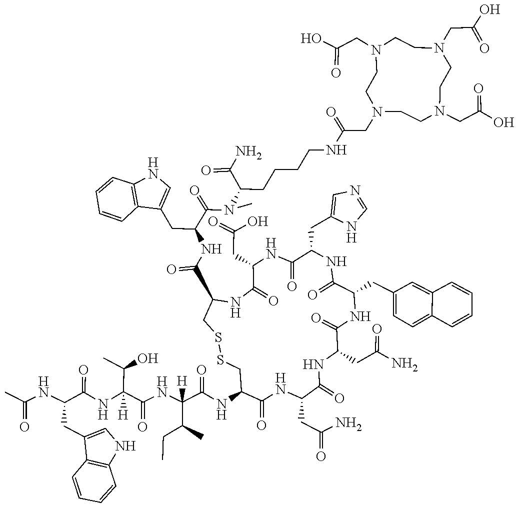 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 352 | 283 |
| C₉₄H₁₂₆N₂₄O₂₅S₂ | | |
| 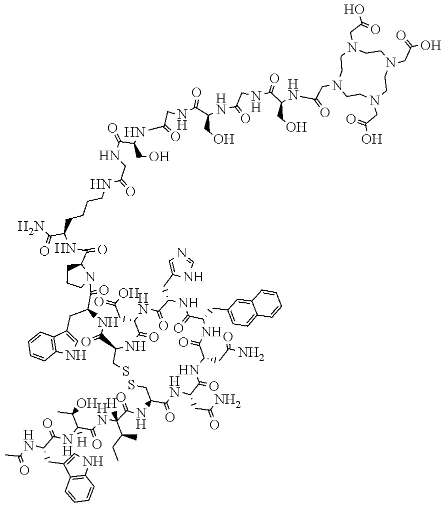 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CNC(=O)[C@H](CO)NC(=O)CNC(=O)[C@H](CO)NC(=O)CNC(=O)[C@H](CO)NC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 353 | 284 |
| C₁₁₃H₁₅₅N₃₁O₃₅S₂ | | |
| 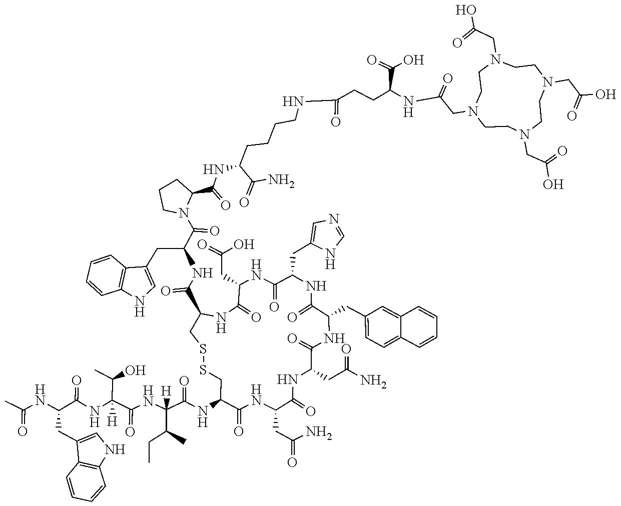 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CC[C@H](NC(=O)CN2CCN(CC(=O)O)CCN(CC(=O)O)CCN(CC(=O)O)CC2)C(=O)O)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 354 | 285 |
| C₁₀₃H₁₃₈N₂₆O₂₉S₂ | | |
| 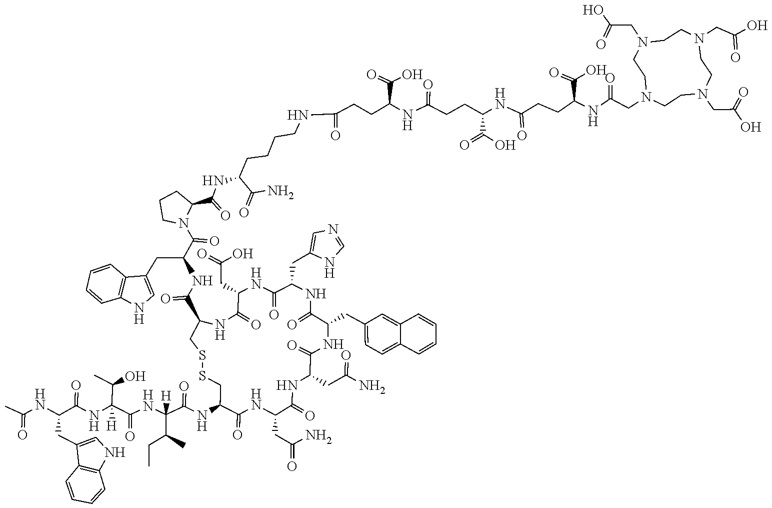 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 355 | 286 |
| C₁₁₃H₁₅₂N₂₈O₃₅S₂ | | |
| 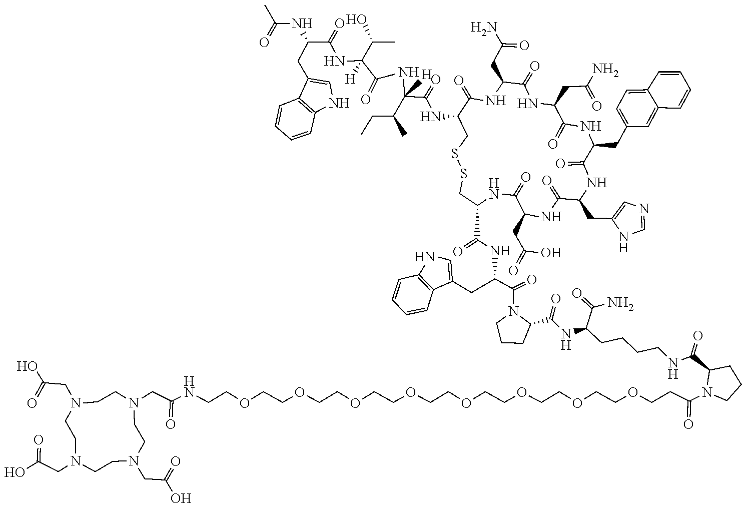 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)[C@H]2CCCN2C(=O)CCOCCOCCOCCOCCOCCOCCOCCOCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 356 | 287 |
| C₁₂₂H₁₇₅N₂₇O₃₆S₂ | | |
| 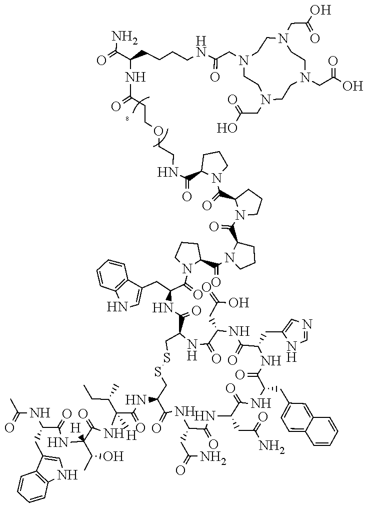 | 357 | 288 |
| C₁₃₂H₁₉₀N₂₉O₃₈S₂ | | |
| 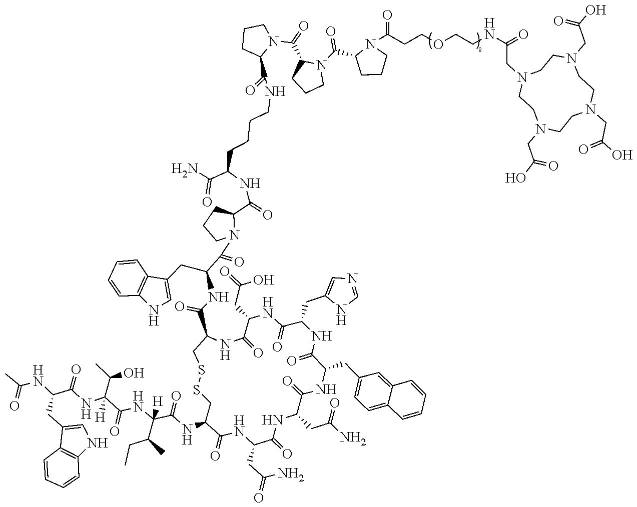 | 358 | 289 |
| C₁₃₂H₁₈₉N₂₉O₃₈S₂ | | |
| 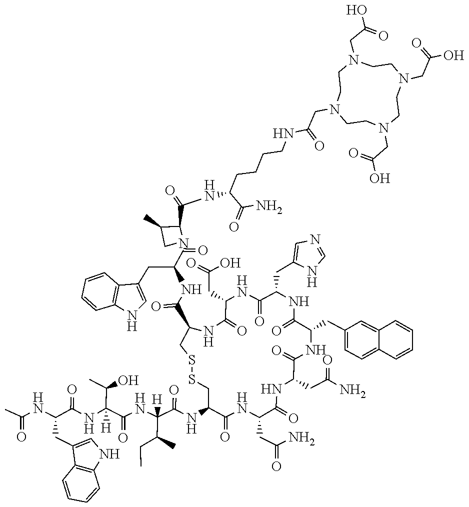 | 359 | 290 |
| C₉₈H₁₃₁N₂₅O₂₆S₂ | | |
| 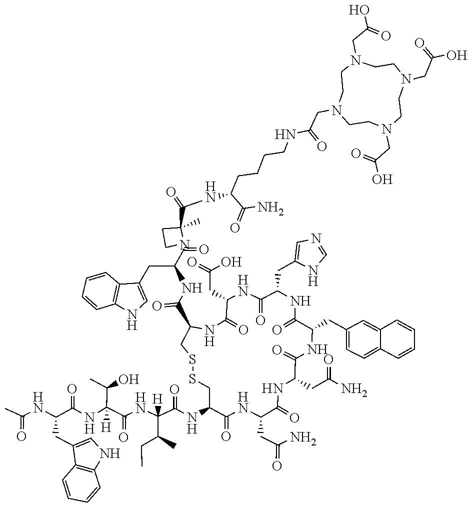 | 360 | 291 |
| C₉₈H₁₃₁N₂₅O₂₆S₂ | | |
| 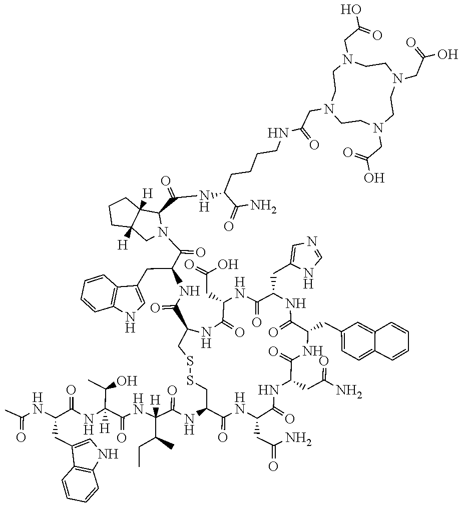 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@@H]3CCC[C@@H]3[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 361 | 292 |
| C₁₀₁H₁₃₅N₂₅O₂₆S₂ | | |
| 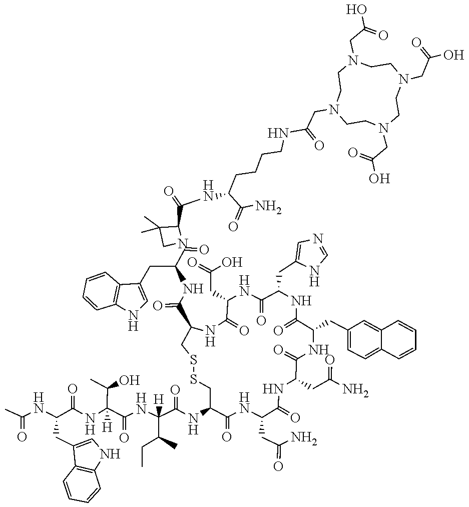 | 362 | 293 |
| C₉₉H₁₃₃N₂₅O₂₆S₂ | | |
| 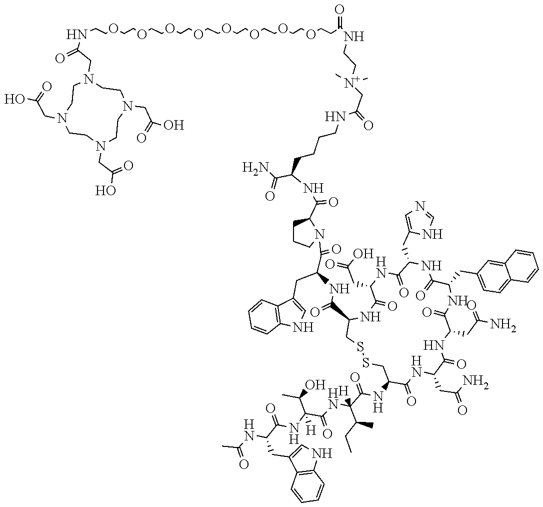 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@H](CC(=O)N2CCC[C@H]2C(=O)N[C@H](CCCCNC(=O)C[N+](C)(C)CCNC(=O)CCCOCCOCCOCCOCCOCCOCCOCCOCCNC(=O)CN2CCN(CC(=O)O)CCN(CC(=O)O)CCN(CC(=O)O)CC2)C(N)=O)Cc2c[nH]c3ccccc23)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 363 | 294 |
| C₁₂₃H₁₈₁N₂₈O₃₆S₂⁺ | | |
| 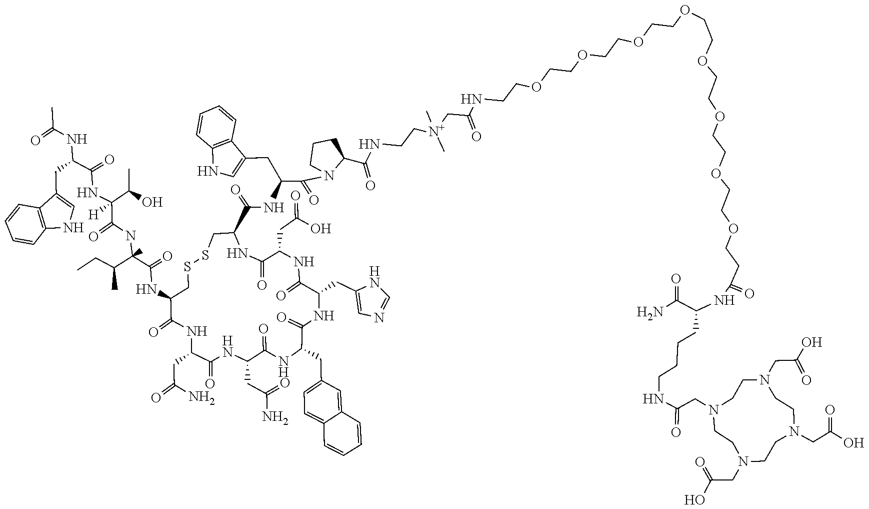 | 364 | 295 |
| C₁₂₃H₁₈₁N₂₈O₃₆S₂⁺ | | |
| 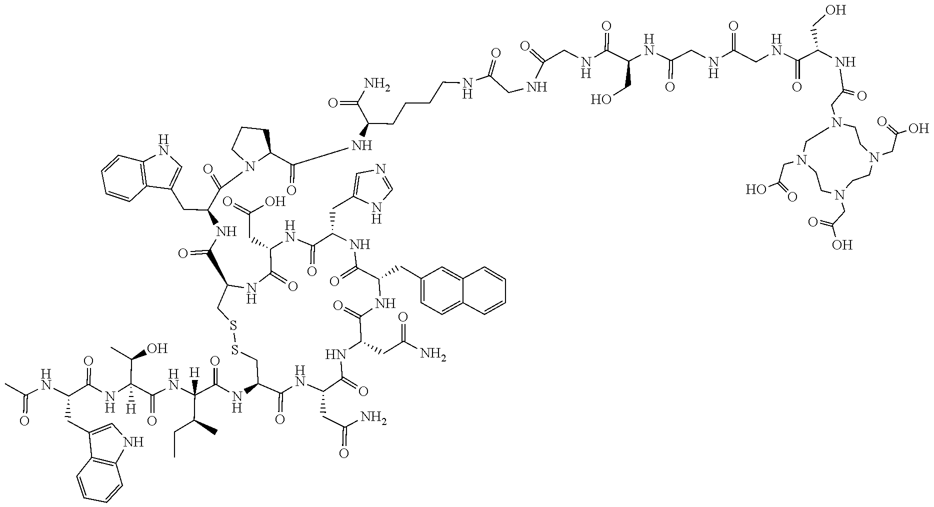 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CNC(=O)CNC(=O)[C@H](CO)NC(=O)CNC(=O)CNC(=O)[C@H](CO)NC(=O)CN2CCN(CC(=O)O)CCN(CC(=O)O)CCN(CC(=O)O)C2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 365 | 296 |
| C₁₁₂H₁₅₃N₃₁O₃₄S₂ | | |
| 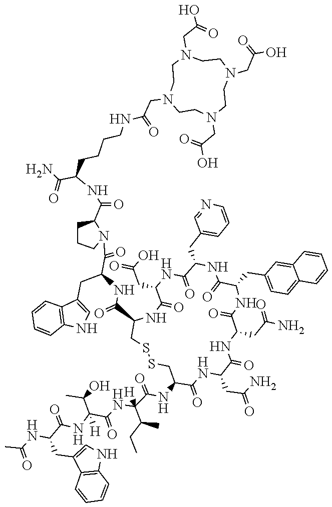 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cccnc2)NC(=O)[C@H](Cc2ccc3ccccc3c2)NC(=O)[C@H](CC(N)=O)NC(=O)[C@H](CC(N)=O)NC1=O] | 366 | 297 |
| C₁₀₀H₁₃₂N₂₄O₂₆S₂ | | |
| 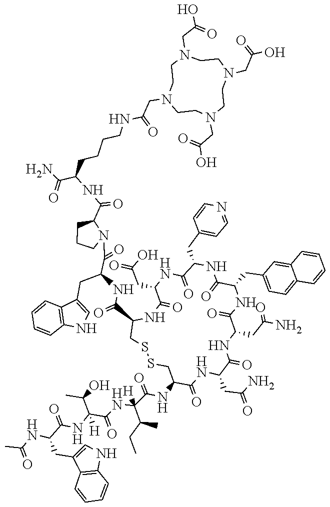 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)CN2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2ccncc2)NC(=O)[C@H](Cc2ccc3ccccc3c2)NC(=O)[C@H](CC(N)=O)NC(=O)[C@H](CC(N)=O)NC1=O] | 367 | 298 |
| C₁₀₀H₁₃₂N₂₄O₂₆S₂ | | |
| 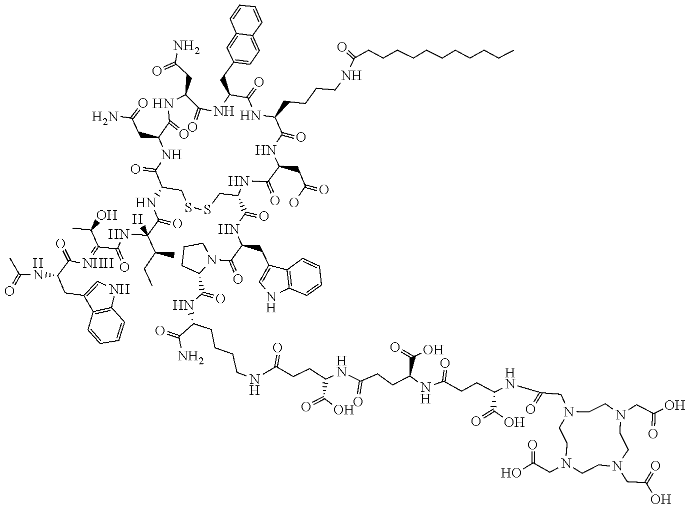 | 368 | 299 |
| C₁₂₅H₁₇₉N₂₇O₃₆S₂ | | |
| 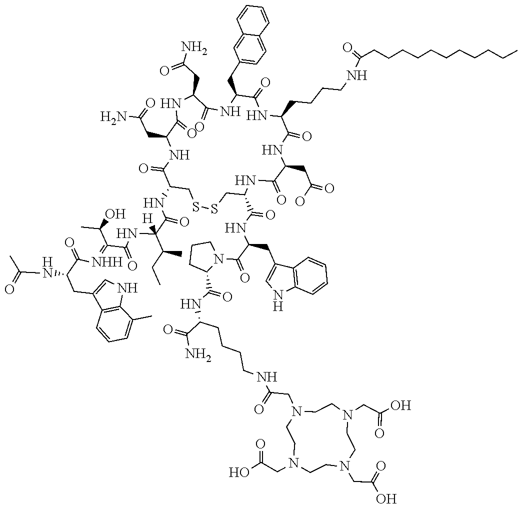 | 369 | 300 |
| C₁₁₁H₁₆₂N₂₄O₂₇S₂ | | |
| 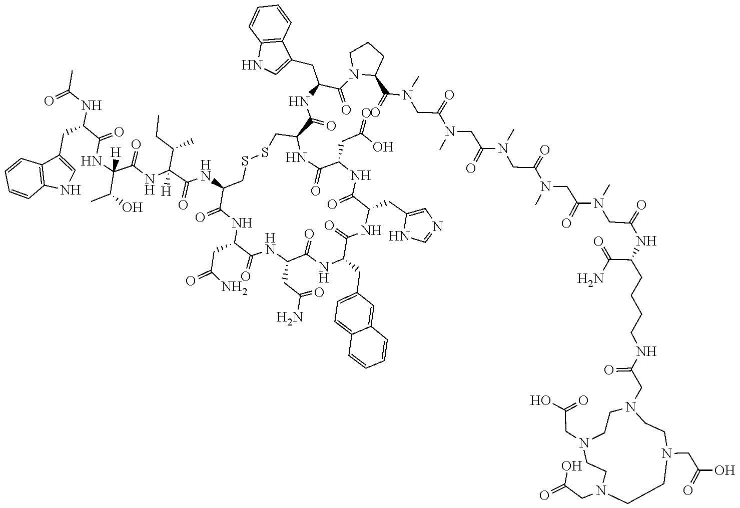 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N(C)CC(=O)N(C)CC(=O)N(C)CC(=O)N(C)CC(=O)N(C)CC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 371 | 301 |
| C₁₁₃H₁₅₆N₃₀O₃₁S₂ | | |
| 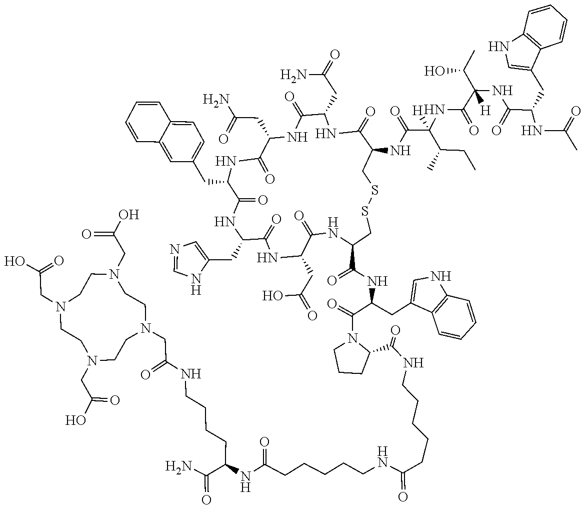 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCCCCCC(=O)NCCCCCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 372 | 302 |
| C₁₁₀H₁₅₅N₂₇O₂₈S₂ | | |
| 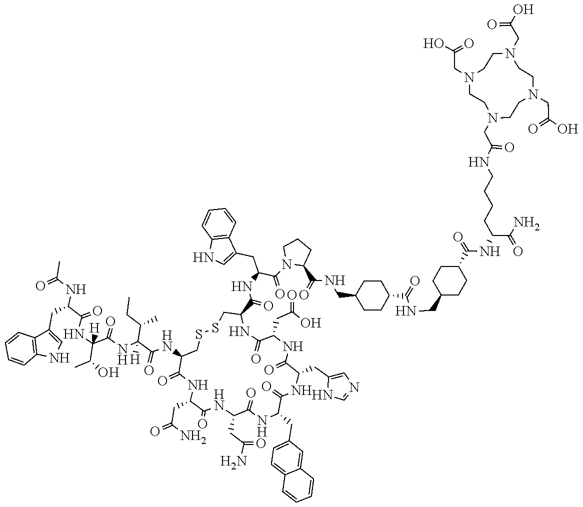 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NC[C@H]2CC[C@H](C(=O)NC[C@H]3CC[C@H](C(=O)N[C@H](CCCCNC(=O)CN4CCN(CC(=O)O)CCN(CC(=O)O)CCN(CC(=O)O)CC4)C(N)=O)CC3)CC2)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O] | 373 | 303 |
| C₁₁₄H₁₅₇N₂₇O₂₈S₂ | | |
| 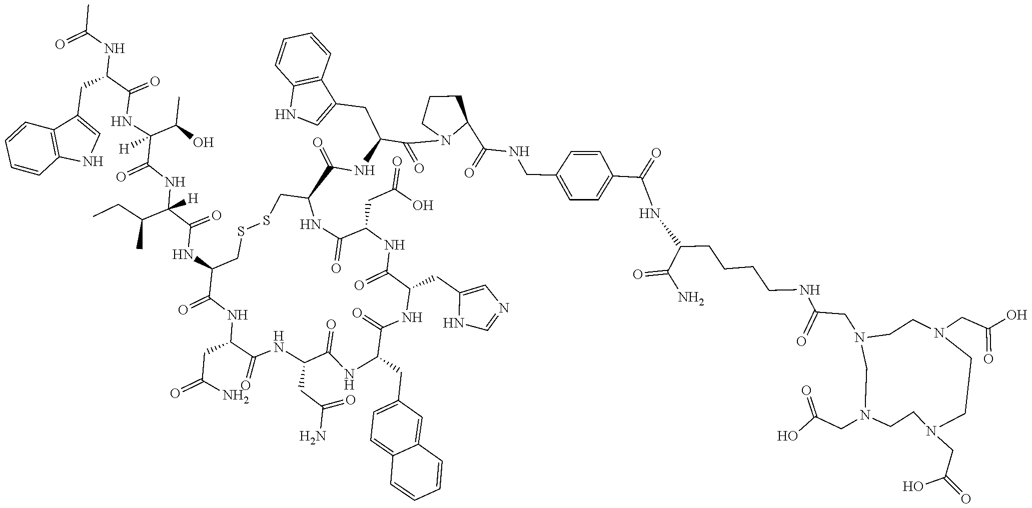 | 374 | 304 |
| C₁₀₆H₁₃₈N₂₆O₂₇S₂ | | |
| 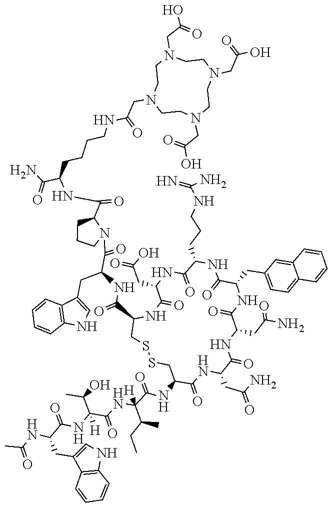 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCCNC(=N)N)NC(=O)[C@H](Cc2ccc3ccccc3c2)NC(=O)[C@H](CC(N)=O)NC(=O)[C@H](CC(N)=O)NC1=O] | 375 | 305 |
| C₉₈H₁₃₆N₂₆O₂₆S₂ | | |
| 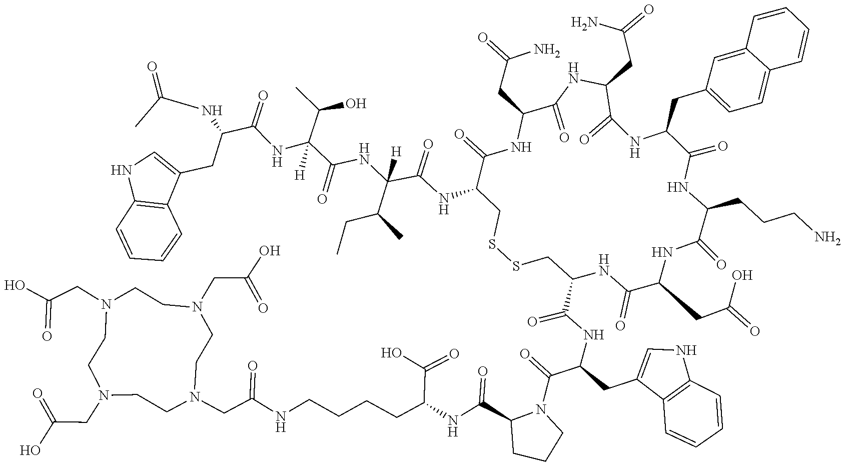 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(=O)O)NC(=O)[C@H](CC(=O)O)NC(=O)[C@H](CCCN)NC(=O)[C@H](Cc2ccc3ccccc3c2)NC(=O)[C@H](CC(N)=O)NC(=O)[C@H](CC(N)=O)NC1=O] | 376 | 306 |
| C₉₇H₁₃₃N₂₃O₂₇S₂ | | |
| 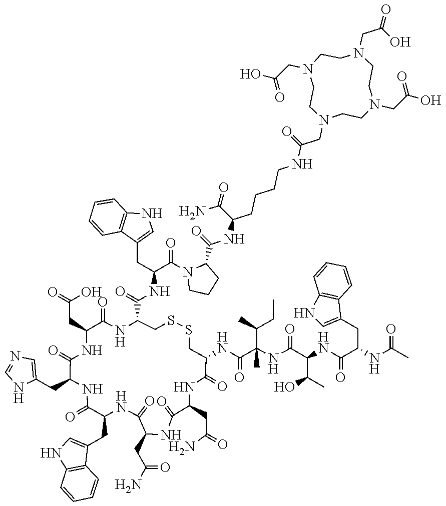 [SMILES: CC[C@H](C)[C@](C)(NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 409 | 307 |
| C₉₇H₁₃₂N₂₆O₂₆S₂ | | |
| 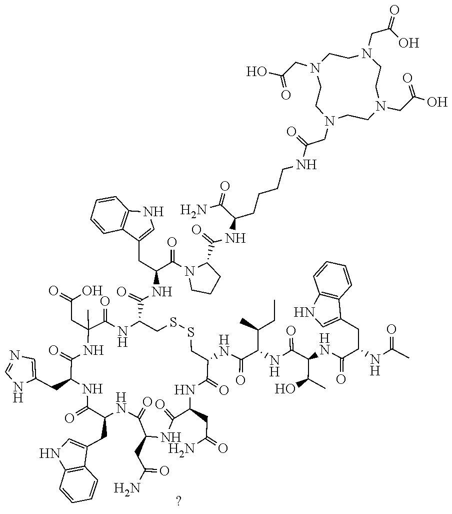 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)C(C)(CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O] | 410 | 308 |
| C₉₇H₁₃₂N₂₆O₂₆S₂ | | |
| 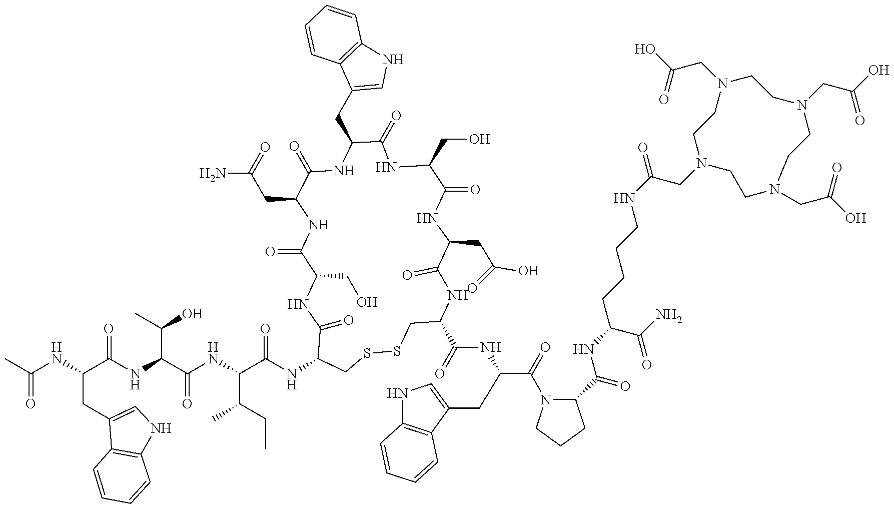 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](Cc2c[nH]c3ccccc23)NC(=O)[C@H](CC(N)=O)NC(=O)[C@H](CO)NC1=O] | 411 | 309 |
| C₉₂H₁₂₇N₂₃O₂₇S₂ | | |
| 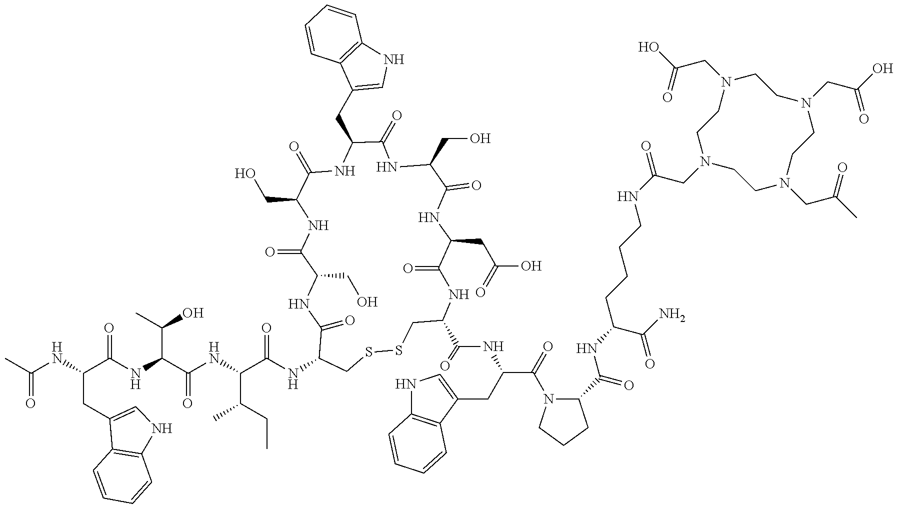 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(C)=O)CCN(CC(=O)O)CCN(CC(=O)O)CC2)C(N)=O)NC(=O)[C@H](CC(=O)O)NC(=O)[C@H](CO)NC(=O)[C@H](Cc2c[nH]c3ccccc23)NC(=O)[C@H](CO)NC(=O)[C@H](CO)NC1=O] | 412 | 310 |
| C₉₁H₁₂₆N₂₂O₂₇S₂ | | |
| 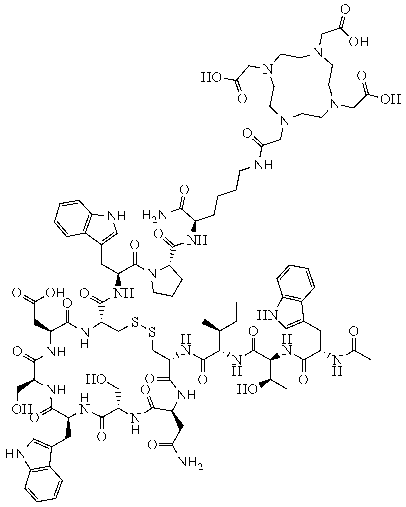 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](Cc2c[nH]c3ccccc23)NC(=O)[C@H](CO)NC(=O)[C@H](CC(N)=O)NC1=O] | 413 | 311 |
| C₉₂H₁₂₇N₂₃O₂₇S₂ | | |
| 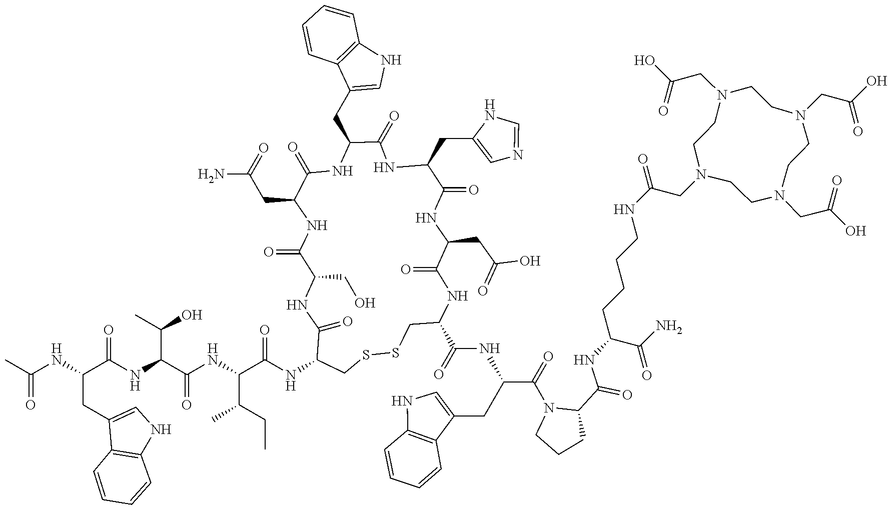 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CO)NC1=O] | 414 | 312 |
| C₉₅H₁₂₉N₂₅O₂₆S₂ | | |
| 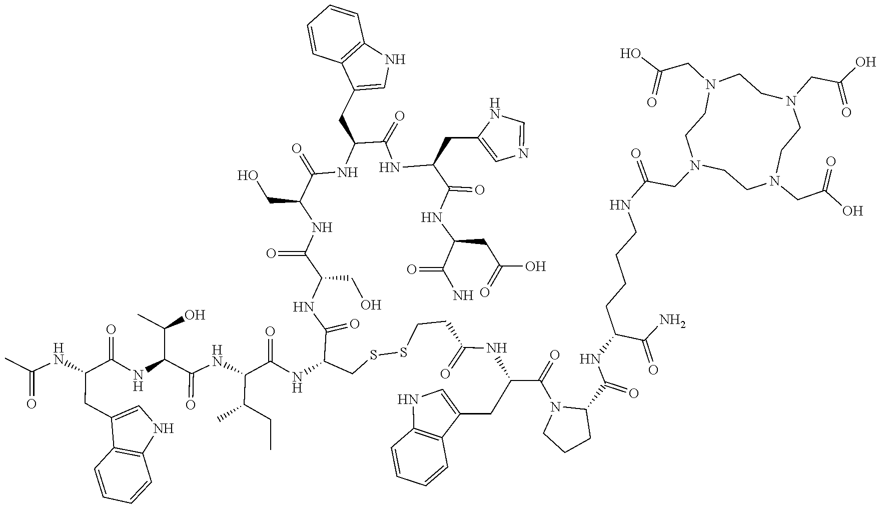 | 415 | 313 |
| C₉₄H₁₂₈N₂₄O₂₆S₂ | | |
| 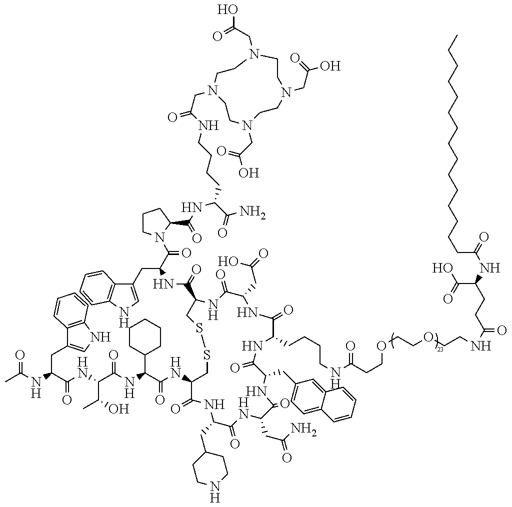 | 426 | 314 |
| C₁₇₆H₂₈₄N₂₆O₅₄S₂ | | |
| 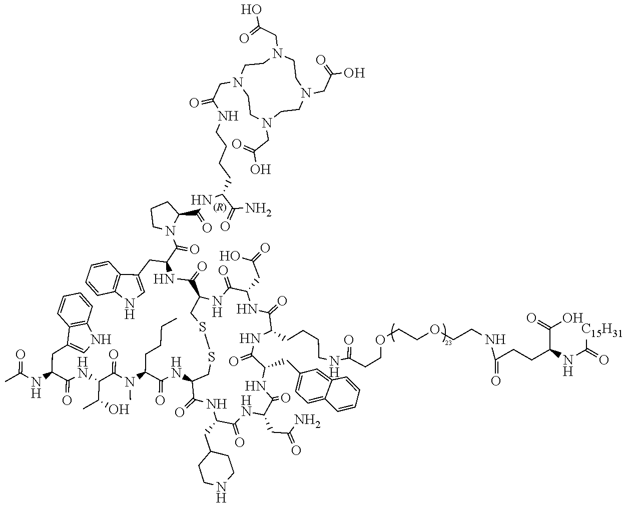 | 427 | 315 |
| C₁₇₅H₂₈₄N₂₆O₅₄S₂ | | |
| 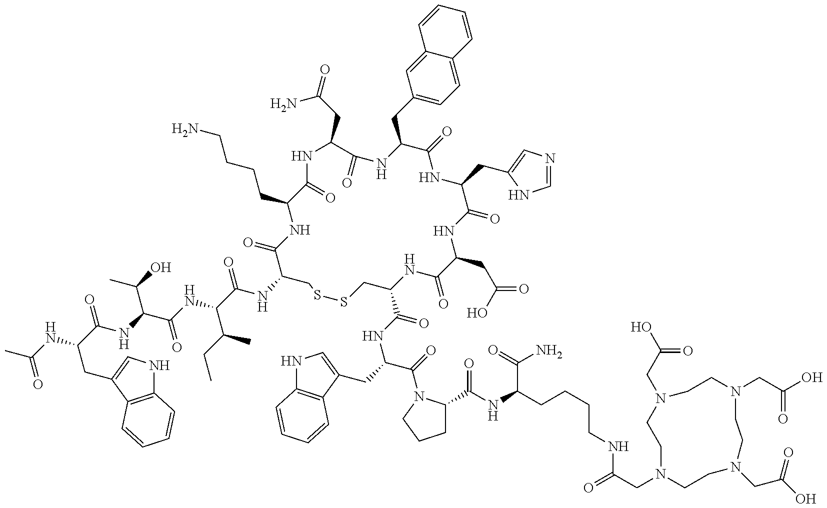 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CCCCN)NC1=O] | 451 | 316 |
| C₁₀₀H₁₃₇N₂₅O₂₅S₂ | | |

In yet another embodiment, the cyclic peptide of Formula B is selected from a peptide in Table B.

TABLE B

| | | SEQ |
|---|---|---|
| Cpd | | ID |
| Nos. | Sequence | Nos. |
| 23 | Ac-W-T-I-C(3)-N-N-W-H-D-C(3)-W-P-[D-Lys(DOTA)]-NH2 | 91 |
| 47 | Ac-W-T-I-C(3)-N-N-W-H-D-C(3)-W-P-(D-Lys)-Ahx-DOTA-NH2 | 92 |
| 48 | Ac-W-T-I-C(3)-N-N-W-H-D-C(3)-W-P-(D-Lys)-CyHex-DOTA-NH2 | 93 |
| 74 | Ac-W-T-I-C(3)-N-N-(1Nal)-H-D-C(3)-W-P-[D-Lys(DOTA)]-NH2 | 94 |
| 75 | Ac-W-T-I-C(3)-N-N-(2Nal)-H-D-C(3)-W-P-[D-Lys(DOTA)]-NH2 | 95 |
| 76 | Ac-W-T-I-C(3)-N-N-W-H-D-C(3)-(1Nal)-P-[D-Lys(DOTA)]-NH2 | 96 |
| 77 | Ac-W-T-I-C(3)-N-N-W-H-D-C(3)-(2Nal)-P-[D-Lys(DOTA)]-NH2 | 97 |
| 85 | Ac-W-T-I-C(3)-N-N-(7Aza-W)-H-D-C(3)-W-P-[D-Lys(DOTA)]-NH2 | 98 |
| 86 | Ac-W-T-I-C(3)-N-N-(5Ome-W)-H-D-C(3)-W-P-[D-Lys(DOTA)]-NH2 | 99 |
| 87 | Ac-W-T-I-C(3)-N-N-W-H-D-C(3)-(7Aza-W)-P-[D-Lys(DOTA)]-NH2 | 100 |
| 88 | Ac-W-T-I-C(3)-N-N-W-H-D-C(3)-(5Ome-W)-P-[D-Lys(DOTA)]-NH2 | 101 |
| 89 | Ac-W-T-I-C(3)-N-N-W-H-D-C(3)-W-P-(D-Lys)-PEG8-DOTA-NH2 | 102 |
| 90 | Ac-W-T-I-C(3)-(DAB-4-NHCOC5H11)-N-W-H-D-C(3)-W-P-[D-Lys(DOTA)]- | 103 |
| | NH2 | |
| 91 | Ac-W-T-I-C(3)-(DAB-4-NHCOC7H15)-N-W-H-D-C(3)-W-P-[D-Lys(DOTA)]- | 104 |
| | NH2 | |
| 92 | Ac-W-T-I-C(3)-N-N-W-(DAB-4-NHCOC5H11)-D-C(3)-W-P-[D-Lys(DOTA)]- | 105 |
| | NH2 | |
| 93 | Ac-W-T-I-C(3)-N-N-W-(DAB-4-NHCOC7H15)-D-C(3)-W-P-[D-Lys(DOTA)]- | 106 |
| | NH2 | |
| 100 | Ac-(5Fluoro-W)-T-I-C(3)-N-N-W-H-D-C(3)-W-P-[D-Lys(DOTA)]-NH2 | 107 |
| 101 | Ac-W-T-I-C(3)-N-N-(5Fluoro-W)-H-D-C(3)-W-P-[D-Lys(DOTA)]-NH2 | 108 |
| 105 | Ac-W-T-I-C(3)-N-N-W-H-D-C(3)-W-(aMe-P)-[D-Lys(DOTA)]-NH2 | 109 |
| 106 | Ac-W-T-(Chg)-C(3)-N-N-W-H-D-C(3)-W-P-[D-Lys(DOTA)]-NH2 | 110 |
| 107 | Ac-W-T-(1Nal)-C(3)-N-N-W-H-D-C(3)-W-P-[D-Lys(DOTA)]-NH2 | 111 |
| 108 | Ac-W-T-I-C(3)-N-N-W-H-D-C(3)-W-(Pip)-[D-Lys(DOTA)]-NH2 | 112 |
| 109 | Ac-W-T-I-C(3)-N-N-W-H-D-C(3)-W-(5,5-diMe-P)-[D-Lys(DOTA)]-NH2 | 113 |
| 110 | Ac-W-T-I-C(3)-N-N-W-H-D-C(3)-W-(cis4OH-P)-[D-Lys(DOTA)]-NH2 | 114 |
| 111 | Ac-W-T-I-C(3)-N-N-W-H-D-C(3)-W-(trans4OH-P)-[D-Lys(DOTA)]-NH2 | 115 |
| 112 | Ac-W-T-I-C(3)-N-N-W-H-D-C(3)-W-(Cis4Fluoro-P)-[D-Lys(DOTA)]-NH2 | 116 |
| 113 | Ac-W-T-I-C(3)-N-N-W-H-D-C(3)-W-(trans4Fluoro-P)-[D-Lys(DOTA)]-NH2 | 117 |
| 114 | Ac-W-T-(2CF3-F)-C(3)-N-N-W-H-D-C(3)-W-P-[D-Lys(DOTA)]-NH2 | 118 |
| 115 | Ac-W-T-(2PhEt-A)-C(3)-N-N-W-H-D-C(3)-W-P-[D-Lys(DOTA)]-NH2 | 119 |
| 116 | Ac-W-T-(2Nal)-C(3)-N-N-W-H-D-C(3)-W-P-[D-Lys(DOTA)]-NH2 | 120 |
| 117 | Ac-W-T-I-C(3)-N-N-W-H-D-C(3)-(aMe-W)-P-[D-Lys(DOTA)]-NH2 | 121 |
| 118 | Ac-W-T-I-C(3)-N-N-(aMe-W)-H-D-C(3)-W-P-[D-Lys(DOTA)]-NH2 | 122 |
| 119 | C15-(L-gE)-PEG12-W-T-I-C(3)-N-N-W-H-D-C(3)-W-P-[D-Lys(DOTA)]-NH2 | 123 |
| 121 | Ac-(5Fluoro-W)-T-(Tbg)-C(3)-N-N-(4Fluoro-F)-H-D-C(3)-(7Aza-W)-(Aze)- | 124 |
| | [D-Lys(DOTA)]-NH2 | |
| 122 | Ac-(5Fluoro-W)-T-(Env)-C(3)-N-N-(4Fluoro-F)-H-D-C(3)-(7Aza-W)-(Aze)- | 125 |
| | [D-Lys(DOTA)]-NH2 | |
| 123 | Ac-(5Fluoro-W)-T-(THPG)-C(3)-N-N-(4Fluoro-F)-H-D-C(3)-(7Aza-W)-(Aze)- | 126 |
| | [D-Lys(DOTA)]-NH2 | |
| 124 | Ac-(5Fluoro-W)-T-(Nle)-C(3)-N-N-(4Fluoro-F)-H-D-C(3)-(7Aza-W)-(Aze)- | 127 |
| | [D-Lys(DOTA)]-NH2 | |
| 125 | Ac-(5Fluoro-W)-T-(CHA)-C(3)-N-N-(4Fluoro-F)-H-D-C(3)-(7Aza-W)-(Aze)- | 128 |
| | [D-Lys(DOTA)]-NH2 | |
| 126 | Ac-(5Fluoro-W)-T-(CBA)-C(3)-N-N-(4Fluoro-F)-H-D-C(3)-(7Aza-W)-(Aze)- | 129 |
| | [D-Lys(DOTA)]-NH2 | |
| 127 | Ac-(5Fluoro-W)-T-(Chg)-C(3)-N-N-(4Fluoro-F)-H-D-C(3)-(7Aza-W)-(Aze)- | 130 |
| | [D-Lys(DOTA)]-NH2 | |
| 128 | Ac-(5Fluoro-W)-T-(2CF3-F)-C(3)-N-N-(4Fluoro-F)-H-D-C(3)-(7Aza-W)- | 131 |
| | (Aze)-[D-Lys(DOTA)]-NH2 | |
| 129 | Ac-(5Fluoro-W)-T-(Tbg)-C(3)-N-N-(4Fluoro-F)-H-D-C(3)-(7Aza-W)- | 132 |
| | (trans4Fluoro-P)-[D-Lys(DOTA)]-NH2 | |
| 130 | Ac-(5Fluoro-W)-T-(Env)-C(3)-N-N-(4Fluoro-F)-H-D-C(3)-(7Aza-W)- | 133 |
| | (trans4Fluoro-P)-[D-Lys(DOTA)]-NH2 | |
| 131 | Ac-(5Fluoro-W)-T-(THPG)-C(3)-N-N-(4Fluoro-F)-H-D-C(3)-(7Aza-W)- | 134 |
| | (trans4Fluoro-P)-[D-Lys(DOTA)]-NH2 | |
| 132 | Ac-(5Fluoro-W)-T-(Nle)-C(3)-N-N-(4Fluoro-F)-H-D-C(3)-(7Aza-W)- | 135 |
| | (trans4Fluoro-P)-[D-Lys(DOTA)]-NH2 | |
| 133 | Ac-(5Fluoro-W)-T-(CHA)-C(3)-N-N-(4Fluoro-F)-H-D-C(3)-(7Aza-W)- | 136 |
| | (trans4Fluoro-P)-[D-Lys(DOTA)]-NH2 | |
| 134 | Ac-(5Fluoro-W)-T-(CBA)-C(3)-N-N-(4Fluoro-F)-H-D-C(3)-(7Aza-W)- | 137 |
| | (trans4Fluoro-P)-[D-Lys(DOTA)]-NH2 | |
| 135 | Ac-(5Fluoro-W)-T-(Chg)-C(3)-N-N-(4Fluoro-F)-H-D-C(3)-(7Aza-W)- | 138 |
| | (trans4Fluoro-P)-[D-Lys(DOTA)]-NH2 | |
| 136 | Ac-(5Fluoro-W)-T-(2CF3-F)-C(3)-N-N-(4Fluoro-F)-H-D-C(3)-(7Aza-W)- | 139 |
| | (trans4Fluoro-P)-[D-Lys(DOTA)]-NH2 | |
| 137 | Ac-(5Fluoro-W)-T-(Tbg)-C(3)-N-N-(4CF3-F)-H-D-C(3)-(7Aza-W)-(Aze)-[D- | 140 |
| | Lys(DOTA)]-NH2 | |
| 138 | Ac-(5Fluoro-W)-T-(Env)-C(3)-N-N-(4CF3-F)-H-D-C(3)-(7Aza-W)-(Aze)-[D- | 141 |
| | Lys(DOTA)]-NH2 | |
| 139 | Ac-(5Fluoro-W)-T-(THPG)-C(3)-N-N-(4CF3-F)-H-D-C(3)-(7Aza-W)-(Aze)- | 142 |
| | [D-Lys(DOTA)]-NH2 | |
| 140 | Ac-(5Fluoro-W)-T-(Nle)-C(3)-N-N-(4CF3-F)-H-D-C(3)-(7Aza-W)-(Aze)-[D- | 143 |
| | Lys(DOTA)]-NH2 | |
| 141 | Ac-(5Fluoro-W)-T-(CHA)-C(3)-N-N-(4CF3-F)-H-D-C(3)-(7Aza-W)-(Aze)-[D- | 144 |
| | Lys(DOTA)]-NH2 | |
| 142 | Ac-(5Fluoro-W)-T-(CBA)-C(3)-N-N-(4CF3-F)-H-D-C(3)-(7Aza-W)-(Aze)-[D- | 145 |
| | Lys(DOTA)]-NH2 | |
| 143 | Ac-(5Fluoro-W)-T-(Chg)-C(3)-N-N-(4CF3-F)-H-D-C(3)-(7Aza-W)-(Aze)-[D- | 146 |
| | Lys(DOTA)]-NH2 | |
| 144 | Ac-(5Fluoro-W)-T-(2CF3-F)-C(3)-N-N-(4CF3-F)-H-D-C(3)-(7Aza-W)-(Aze)- | 147 |
| | [D-Lys(DOTA)]-NH2 | |
| 145 | Ac-(5Fluoro-W)-T-(Tbg)-C(3)-N-N-(4CF3-F)-H-D-C(3)-(7Aza-W)- | 148 |
| | (trans4Fluoro-P)-[D-Lys(DOTA)]-NH2 | |
| 146 | Ac-(5Fluoro-W)-T-(Env)-C(3)-N-N-(4CF3-F)-H-D-C(3)-(7Aza-W)- | 149 |
| | (trans4Fluoro-P)-[D-Lys(DOTA)]-NH2 | |
| 147 | Ac-(5Fluoro-W)-T-(THPG)-C(3)-N-N-(4CF3-F)-H-D-C(3)-(7Aza-W)- | 150 |
| | (trans4Fluoro-P)-[D-Lys(DOTA)]-NH2 | |
| 148 | Ac-(5Fluoro-W)-T-(Nle)-C(3)-N-N-(4CF3-F)-H-D-C(3)-(7Aza-W)- | 151 |
| | (trans4Fluoro-P)-[D-Lys(DOTA)]-NH2 | |
| 149 | Ac-(5Fluoro-W)-T-(CHA)-C(3)-N-N-(4CF3-F)-H-D-C(3)-(7Aza-W)- | 152 |
| | (trans4Fluoro-P)-[D-Lys(DOTA)]-NH2 | |
| 150 | Ac-(5Fluoro-W)-T-(CBA)-C(3)-N-N-(4CF3-F)-H-D-C(3)-(7Aza-W)- | 153 |
| | (trans4Fluoro-P)-[D-Lys(DOTA)]-NH2 | |
| 151 | Ac-(5Fluoro-W)-T-(Chg)-C(3)-N-N-(4CF3-F)-H-D-C(3)-(7Aza-W)- | 154 |
| | (trans4Fluoro-P)-[D-Lys(DOTA)]-NH2 | |
| 152 | Ac-(5Fluoro-W)-T-(2CF3-F)-C(3)-N-N-(4CF3-F)-H-D-C(3)-(7Aza-W)- | 155 |
| | (trans4Fluoro-P)-[D-Lys(DOTA)]-NH2 | |
| 156 | Ac-(7-Me-W)-T-I-C(3)-N-N-W-H-D-C(3)-W-P-[D-Lys(DOTA)]-NH2 | 156 |
| 157 | Ac-W-T-I-C(3)-N-N-W-H-D-C(3)-W-NmeS-[D-Lys(DOTA)]-NH2 | 157 |
| 158 | Ac-W-T-I-C(3)-N-N-W-K(C12)-D-C(3)-W-P-[D-Lys(DOTA)]-NH2 | 158 |
| 159 | Ac-W-T-I-C(3)-N-N-W-K(C14)-D-C(3)-W-P-[D-Lys(DOTA)]-NH2 | 159 |
| 160 | Ac-W-T-I-C(3)-N-N-W-K(C16)-D-C(3)-W-P-[D-Lys(DOTA)]-NH2 | 160 |
| 161 | C14-W-T-I-C(3)-N-N-W-H-D-C(3)-W-P-[D-Lys(DOTA)]-NH2 | 161 |
| 162 | C16-W-T-I-C(3)-N-N-W-H-D-C(3)-W-P-[D-Lys(DOTA)]-NH2 | 162 |
| 164 | Ac-WTI-C(1)-NNWHD-C(1)-WP-[D-Lys(DOTA)]-NH2 | 163 |
| 165 | Ac-WTI-C(1)-NN(2Nal)HD-C(1)-WP-[D-Lys(DOTA)]-NH2 | 164 |
| 166 | Ac-W-T-I-C(3)-N-N-(2Nal)-H-D-C(3)-W-P-[L-Lys(DOTA)]-NH2 | 165 |
| 167 | Ac-W-T-I-C(3)-N-N-(2Nal)-H-D-C(3)-W-P-G-G-TTDS-[D-Lys(DOTA)]-NH2 | 166 |
| 168 | Ac-(5Fluoro-W)-T-(Tbg)-C(3)-N-N-(1Nal)-H-D-C(3)-(7Aza-W)-(Aze)-[D- | 167 |
| | Lys(DOTA)]-NH2 | |
| 169 | Ac-(5Fluoro-W)-T-(THPG)-C(3)-N-N-(1Nal)-H-D-C(3)-(7Aza-W)-(Aze)-[D- | 168 |
| | Lys(DOTA)]-NH2 | |
| 170 | Ac-(5Fluoro-W)-T-(Nle)-C(3)-N-N-(1Nal)-H-D-C(3)-(7Aza-W)-(Aze)-[D- | 169 |
| | Lys(DOTA)]-NH2 | |
| 171 | Ac-(5Fluoro-W)-T-(CHA)-C(3)-N-N-(1Nal)-H-D-C(3)-(7Aza-W)-(Aze)-[D- | 170 |
| | Lys(DOTA)]-NH2 | |
| 172 | Ac-(5Fluoro-W)-T-(CBA)-C(3)-N-N-(1Nal)-H-D-C(3)-(7Aza-W)-(Aze)-[D- | 171 |
| | Lys(DOTA)]-NH2 | |
| 173 | Ac-(5Fluoro-W)-T-(Chg)-C(3)-N-N-(1Nal)-H-D-C(3)-(7Aza-W)- | 172 |
| | (trans4Fluoro-P)-[D-Lys(DOTA)]-NH2 | |
| 174 | Ac-(5Fluoro-W)-T-(2CF3-F)-C(3)-N-N-(1Nal)-H-D-C(3)-(7Aza-W)- | 173 |
| | (trans4Fluoro-P)-[D-Lys(DOTA)]-NH2 | |
| 175 | Ac-(5Fluoro-W)-T-(Chg)-C(3)-N-N-(1Nal)-H-D-C(3)-(7Aza-W)-(Aze)-[D- | 174 |
| | Lys(DOTA)]-NH2 | |
| 176 | Ac-(5Fluoro-W)-T-(2CF3-F)-C(3)-N-N-(1Nal)-H-D-C(3)-(7Aza-W)-(Aze)-[D- | 175 |
| | Lys(DOTA)]-NH2 | |
| 177 | Ac-(5Fluoro-W)-T-(Tbg)-C(3)-N-N-(1Nal)-H-D-C(3)-(7Aza-W)- | 176 |
| | (trans4Fluoro-P)-[D-Lys(DOTA)]-NH2 | |
| 178 | Ac-(5Fluoro-W)-T-(Env)-C(3)-N-N-(1Nal)-H-D-C(3)-(7Aza-W)- | 177 |
| | (trans4Fluoro-P)-[D-Lys(DOTA)]-NH2 | |
| 179 | Ac-(5Fluoro-W)-T-(THPG)-C(3)-N-N-(1Nal)-H-D-C(3)-(7Aza-W)- | 178 |
| | (trans4Fluoro-P)-[D-Lys(DOTA)]-NH2 | |
| 180 | Ac-(5Fluoro-W)-T-(Nle)-C(3)-N-N-(1Nal)-H-D-C(3)-(7Aza-W)- | 179 |
| | (trans4Fluoro-P)-[D-Lys(DOTA)]-NH2 | |
| 181 | Ac-(5Fluoro-W)-T-(CHA)-C(3)-N-N-(1Nal)-H-D-C(3)-(7Aza-W)- | 180 |
| | (trans4Fluoro-P)-[D-Lys(DOTA)]-NH2 | |
| 182 | Ac-(5Fluoro-W)-T-(CBA)-C(3)-N-N-(1Nal)-H-D-C(3)-(7Aza-W)- | 181 |
| | (trans4Fluoro-P)-[D-Lys(DOTA)]-NH2 | |
| 183 | Ac-(5Fluoro-W)-T-(Tbg)-C(3)-N-N-(2Nal)-H-D-C(3)-(7Aza-W)-(Aze)-[D- | 182 |
| | Lys(DOTA)]-NH2 | |
| 184 | Ac-(5Fluoro-W)-T-(Nle)-C(3)-N-N-(2Nal)-H-D-C(3)-(7Aza-W)-(Aze)-[D- | 183 |
| | Lys(DOTA)]-NH2 | |
| 185 | Ac-(5Fluoro-W)-T-(CBA)-C(3)-N-N-(2Nal)-H-D-C(3)-(7Aza-W)-(Aze)-[D- | 184 |
| | Lys(DOTA)]-NH2 | |
| 186 | Ac-(5Fluoro-W)-T-(Env)-C(3)-N-N-(2Nal)-H-D-C(3)-(7Aza-W)-(Aze)-[D- | 185 |
| | Lys(DOTA)]-NH2 | |
| 187 | Ac-(5Fluoro-W)-T-(Chg)-C(3)-N-N-(2Nal)-H-D-C(3)-(7Aza-W)-(Aze)-[D- | 186 |
| | Lys(DOTA)]-NH2 | |
| 188 | Ac-(5Fluoro-W)-T-(THPG)-C(3)-N-N-(2Nal)-H-D-C(3)-(7Aza-W)-(Aze)-[D- | 187 |
| | Lys(DOTA)]-NH2 | |
| 189 | Ac-(5Fluoro-W)-T-(CHA)-C(3)-N-N-(2Nal)-H-D-C(3)-(7Aza-W)-(Aze)-[D- | 188 |
| | Lys(DOTA)]-NH2 | |
| 190 | Ac-(5Fluoro-W)-T-(2CF3-F)-C(3)-N-N-(2Nal)-H-D-C(3)-(7Aza-W)-(Aze)-[D- | 189 |
| | Lys(DOTA)]-NH2 | |
| 193 | Ac-W-T-(Env)-C(3)-N-N-(2Nal)-H-D-C(3)-(7-Aza-W)-P-[D-Lys(DOTA)]-NH2 | 190 |
| 194 | Ac-W-T-(Chg)-C(3)-A-N-(2Nal)-H-D-C(3)-(7-Aza-W)-P-[D-Lys(DOTA)]-NH2 | 191 |
| 195 | Ac-W-T-(CHA)-C(3)-A-N-(2Nal)-H-D-C(3)-(7-Aza-W)-P-[D-Lys(DOTA)]- | 192 |
| | NH2 | |
| 196 | Ac-W-T-(Env)-C(3)-A-N-(2Nal)-H-D-C(3)-(7-Aza-W)-P-[D-Lys(DOTA)]-NH2 | 193 |
| 197 | Ac-W-T-(Chg)-C(3)-N-A-(2Nal)-H-D-C(3)-(7-Aza-W)-P-[D-Lys(DOTA)]-NH2 | 194 |
| 198 | Ac-W-T-(CHA)-C(3)-N-A-(2Nal)-H-D-C(3)-(7-Aza-W)-P-[D-Lys(DOTA)]- | 195 |
| | NH2 | |
| 199 | Ac-W-T-(Env)-C(3)-N-A-(2Nal)-H-D-C(3)-(7-Aza-W)-P-[D-Lys(DOTA)]-NH2 | 196 |
| 201 | Ac-M-W-T-L-C(3)-D-W-N-S-D-C(3)-W-P-[D-Lys(DOTA)]-NH2 | 197 |
| 202 | Ac-W-T-(Env)-C(3)-N-N-(2Nal)-H-D-C(3)-(7-Aza-W)-P-[D-Lys(Chx-DOTA)]- | 198 |
| | NH2 | |
| 203 | Ac-W-T-(Chg)-C(3)-N-N-(2Nal)-H-D-C(3)-(7-Aza-W)-P-[D-Lys(Chx-DOTA)]- | 199 |
| | NH2 | |
| 204 | Ac-W-T-(Env)-C(3)-N-N-(2Nal)-H-D-C(3)-(7-Aza-W)-P-[D-Lys(G-G-TTDS- | 200 |
| | DOTA)]-NH2 | |
| 205 | Ac-W-(Lys)-I-C(3)-N-N-(2Nal)-H-D-C(3)-W-P-[D-Lys(DOTA)]-NH2 | 201 |
| 206 | Ac-(7-Me-W)-T-I-C(3)-N-N-(2Nal)-H-D-C(3)-W-P-[D-Lys(DOTA)]-NH2 | 202 |
| 207 | Ac-(7-Me-W)-T-I-C(3)-N-N-(2Nal)-H-D-C(3)-W-P-[D-Lys(gE-gE-gE-DOTA)]- | 203 |
| | NH2 | |
| 208 | Ac-W-T-I-C(3)-(Ser)-N-(2Nal)-H-D-C(3)-W-P-[D-Lys(DOTA)]-NH2 | 204 |
| 209 | Ac-W-T-I-C(3)-N-(Asp)-(2Nal)-H-D-C(3)-W-P-[D-Lys(DOTA)]-NH2 | 205 |
| 210 | Ac-W-T-I-C(3)-N-N-(2Nal)-(Gln)-D-C(3)-W-P-[D-Lys(DOTA)]-NH2 | 206 |
| 211 | Ac-W-T-I-C(3)-N-N-(2Nal)-H-(Asn)-C(3)-W-P-[D-Lys(DOTA)]-NH2 | 207 |
| 212 | Ac-W-T-I-C(3)-(Ser)-(Asp)-(2Nal)-(Gln)-(Asn)-C(3)-W-P-[D-Lys(DOTA)]- | 208 |
| | NH2 | |
| 213 | Ac-W-T-I-C(3)-N-(Lys)-(2Nal)-H-D-C(3)-W-P-[D-Lys(DOTA)]-NH2 | 209 |
| 226 | Ac-(7-CI-W)-T-I-C(3)-N-N-W-H-D-C(3)-W-P-[D-Lys(DOTA)]-NH2 | 210 |
| 227 | Ac-W-T-I-C(3)-N-N-W-H-D-C(3)-W-Sar-[D-Lys(DOTA)]-NH2 | 211 |
| 228 | Ac-W-T-I-C(3)-N-N-W-H-D-C(3)-W-Aze-[D-Lys(DOTA)]-NH2 | 212 |
| 229 | Ac-W-T-I-C(3)-N-N-W-H-D-C(3)-W-NmeA-[D-Lys(DOTA)]-NH2 | 213 |
| 230 | Ac-W-T-I-C(3)-N-N-(2Nal)-H-D-C(3)-W-P-[D-Lys(G-G-TTDS-DOTA)]-NH2 | 214 |
| 231 | Ac-W-T-I-C(3)-N-N-(2Nal)-H-D-C(3)-W-P-G-G-S-G-G-S-[D-Lys(DOTA)]- | 215 |
| | NH2 | |
| 232 | Ac-W-T-I-C(3)-N-N-(2Nal)-H-D-C(3)-W-P-G-S-G-S-G-S-[D-Lys(DOTA)]- | 216 |
| | NH2 | |
| 233 | Ac-W-T-I-C(3)-N-N-(2Nal)-H-D-C(3)-W-P-gE-[D-Lys(DOTA)]-NH2 | 217 |
| 234 | Ac-W-T-I-C(3)-N-N-(2Nal)-H-D-C(3)-W-P-gE-gE-gE-[D-Lys(DOTA)]-NH2 | 218 |
| 235 | Ac-W-T-I-C(3)-N-N-(2Nal)-H-D-C(3)-W-P-(D-Pro)-PEG8-[D-Lys(DOTA)]- | 219 |
| | NH2 | |
| 237 | Ac-W-T-I-C(3)-N-N-(2Nal)-K-D-C(3)-W-P-[D-Lys(DOTA)]-NH2 | 220 |
| 238 | Ac-W-T-I-C(3)-N-N-(2Nal)-(K(Me)3)-D-C(3)-W-P-[D-Lys(DOTA)]-NH2 | 221 |
| 241 | Ac-(5Fluoro-W)-T-(Env)-C(3)-N-N-(1Nal)-H-D-C(3)-(7Aza-W)-(Aze)-[D- | 222 |
| | Lys(DOTA)]-NH2 | |
| 242 | Ac-(5Fluoro-W)-T-(Env)-C(3)-N-N-(2Nal)-H-D-C(3)-(7Aza-W)- | 223 |
| | (trans4Fluoro-P)-[D-Lys(DOTA)]-NH2 | |
| 243 | Ac-(5Fluoro-W)-T-(THPG)-C(3)-N-N-(2Nal)-H-D-C(3)-(7Aza-W)- | 224 |
| | (trans4Fluoro-P)-[D-Lys(DOTA)]-NH2 | |
| 244 | Ac-(5Fluoro-W)-T-(Nle)-C(3)-N-N-(2Nal)-H-D-C(3)-(7Aza-W)- | 225 |
| | (trans4Fluoro-P)-[D-Lys(DOTA)]-NH2 | |
| 245 | Ac-(5Fluoro-W)-T-(CHA)-C(3)-N-N-(2Nal)-H-D-C(3)-(7Aza-W)- | 226 |
| | (trans4Fluoro-P)-[D-Lys(DOTA)]-NH2 | |
| 246 | Ac-(5Fluoro-W)-T-(2CF3-F)-C(3)-N-N-(2Nal)-H-D-C(3)-(7Aza-W)- | 227 |
| | (trans4Fluoro-P)-[D-Lys(DOTA)]-NH2 | |
| 251 | Ac-W-T-I-C(3)-N-N-(2Nal)-H-D-C(3)-W-P-{D-Lys[PEG8-D-Lys(DOTA)- | 228 |
| | PEG8-COCH2Ph-4Br]}-NH2 | |
| 252 | Ac-W-T-I-C(3)-N-N-(2Nal)-H-D-C(3)-W-P-{D-Lys[PEG8-D-Lys(DOTA)- | 229 |
| | PEG12-COCH2Ph-4Br]}-NH2 | |
| 255 | Ac-W-T-I-C(3)-N-N-(2Nal)-H-D-C(3)-W-P-{D-Lys[PEG8-D-Lys(DOTA)- | 230 |
| | PEG24-COCH2Ph-4Br]}-NH2 | |
| 256 | Ac-W-T-I-C(3)(pDBX)-N-N-(2Nal)-H-D-C(2)-W-P-[D-Lys(DOTA)]-NH2 | 231 |
| 257 | Ac-(5Fluoro-W)-T-(Tbg)-C(3)-N-N-(2Nal)-H-D-C(3)-(7Aza-W)- | 232 |
| | (trans4Fluoro-P)-[D-Lys(DOTA)]-NH2 | |
| 258 | Ac-(5Fluoro-W)-T-(Chg)-C(3)-N-N-(2Nal)-H-D-C(3)-(7Aza-W)- | 233 |
| | (trans4Fluoro-P)-[D-Lys(DOTA)]-NH2 | |
| 260 | Ac-W-T-I-C(3)-[3-(4-piperidinyl)-Ala]-N-(2Nal)-H-D-C(3)-W-P-[D- | 234 |
| | Lys(DOTA)]-NH2 | |
| 261 | Ac-W-T-I-C(3)-[3-(1-morpholinyl)-Ala]-N-(2Nal)-H-D-C(3)-W-P-[D- | 235 |
| | Lys(DOTA)]-NH2 | |
| 262 | Ac-W-T-I-C(3)-N-N-(2Nal)-H-D-C(3)-W-P-[D-Lys(DOTA)]-OH | 236 |
| 263 | Ac-W-T-I-C(3)-(3Pya)-N-(2Nal)-H-D-C(3)-W-P-[D-Lys(DOTA)]-NH2 | 237 |
| 264 | Ac-W-T-I-C(3)-(4Pya)-N-(2Nal)-H-D-C(3)-W-P-[D-Lys(DOTA)]-NH2 | 238 |
| 265 | Ac-W-T-I-C(3)-N-(3Pya)-(2Nal)-H-D-C(3)-W-P-[D-Lys(DOTA)]-NH2 | 239 |
| 266 | Ac-W-T-I-C(3)-N-(4Pya)-(2Nal)-H-D-C(3)-W-P-[D-Lys(DOTA)]-NH2 | 240 |
| 267 | Ac-W-T-I-C(3)-N-[3-(4-piperidinyl)-Ala]-(2Nal)-H-D-C(3)-W-P-[D- | 241 |
| | Lys(DOTA)]-NH2 | |
| 268 | Ac-W-T-I-C(3)-N-[3-(1-morpholinyl)-Ala]-(2Nal)-H-D-C(3)-W-P-[D- | 242 |
| | Lys(DOTA)]-NH2 | |
| 269 | Ac-(5Fluoro-W)-T-(CBA)-C(3)-N-N-(2Nal)-H-D-C(3)-(7Aza-W)- | 243 |
| | (trans4Fluoro-P)-[D-Lys(DOTA)]-NH2 | |
| 270 | Ac-W-T-I-C(3)-N-N-(2Nal)-H-D-C(3)-W-P-PEG4-[D-Lys(DOTA)]-NH2 | 244 |
| 271 | Ac-W-T-I-C(3)-N-N-(2Nal)-H-D-C(3)-W-P-[D-Lys(PEG4-DOTA)]-NH2 | 245 |
| 272 | Ac-W-T-I-C(3)-N-N-W-H-D-C(3)-(7-CI-W)-P-[D-Lys(DOTA)]-NH2 | 246 |
| 273 | Ac-W-T-I-C(3)-Glu-N-W-Glu-D-C(3)-W-P-[D-Lys(DOTA)]-NH2 | 247 |
| 274 | Ac-W-T-I-C(3)-N-N-W-Glu-D-C(3)-W-P-[D-Lys(DOTA)]-NH2 | 248 |
| 275 | Ac-W-T-I-C(3)-Glu-Glu-W-Glu-D-C(3)-W-P-[D-Lys(DOTA)]-NH2 | 249 |
| 276 | Ac-W-T-I-C(3)-N-Glu-W-Glu-D-C(3)-W-P-[D-Lys(DOTA)]-NH2 | 250 |
| 277 | Ac-W-T-I-C(3)-Glu-N-W-H-D-C(3)-W-P-[D-Lys(DOTA)]-NH2 | 251 |
| 278 | Ac-W-T-I-C(3)-Glu-Glu-W-H-D-C(3)-W-P-[D-Lys(DOTA)]-NH2 | 252 |
| 279 | Ac-W-T-I-C(3)-N-Glu-W-H-D-C(3)-W-P-[D-Lys(DOTA)]-NH2 | 253 |
| 280 | Ac-W-T-(Chg)-C(3)-[3-(4-piperidinyl)-Ala]-N-(2Nal)-H-D-C(3)-W-P-[D- | 254 |
| | Lys(DOTA)]-NH2 | |
| 281 | Ac-W-T-(Tbg)-C(3)-[3-(4-piperidinyl)-Ala]-N-(2Nal)-H-D-C(3)-W-P-[D- | 255 |
| | Lys(DOTA)]-NH2 | |
| 282 | Ac-W-T-(t-Bu-Ala)-C(3)-[3-(4-piperidinyl)-Ala]-N-(2Nal)-H-D-C(3)-W-P- | 256 |
| | [D-Lys(DOTA)]-NH2 | |
| 283 | Ac-W-T-(Chg)-C(3)-[3-(4-piperidinyl)-Ala]-N-(2Nal)-H-(Asn)-C(3)-W-P- | 257 |
| | [D-Lys(DOTA)]-NH2 | |
| 284 | Ac-W-T-(Chg)-C(3)-[3-(4-piperidinyl)-Ala]-N-(2Nal)-H-(Ala)-C(3)-W-P- | 258 |
| | [D-Lys(DOTA)]-NH2 | |
| 285 | Ac-W-T-(Chg)-C(3)-[3-(4-piperidinyl)-Ala]-N-(2Nal)-H-(Thr)-C(3)-W-P- | 259 |
| | [D-Lys(DOTA)]-NH2 | |
| 286 | Ac-W-T-(Chg)-C(3)-[3-(4-piperidinyl)-Ala]-N-(2Nal)-H-D-C(3)-W-P-[D- | 260 |
| | Lys(Chx-DOTA)]-NH2 | |
| 287 | Ac-W-T-(Chg)-C(3)-(3Pya)-N-(2Nal)-H-D-C(3)-W-P-[D-Lys(DOTA)]-NH2 | 261 |
| 288 | Ac-W-T-(Tbg)-C(3)-(3Pya)-N-(2Nal)-H-D-C(3)-W-P-[D-Lys(DOTA)]-NH2 | 262 |
| 289 | Ac-W-T-(t-Bu-Ala)-C(3)-(3Pya)-N-(2Nal)-H-D-C(3)-W-P-[D-Lys(DOTA)]- | 263 |
| | NH2 | |
| 290 | Ac-W-T-(Chg)-C(3)-(4Pya)-N-(2Nal)-H-D-C(3)-W-P-[D-Lys(DOTA)]-NH2 | 264 |
| 291 | Ac-W-T-(Tbg)-C(3)-(4Pya)-N-(2Nal)-H-D-C(3)-W-P-[D-Lys(DOTA)]-NH2 | 265 |
| 292 | Ac-W-T-I-C(3)-N-N-(5OH-W)-H-D-C(3)-W-P-[D-Lys(DOTA)]-NH2 | 266 |
| 293 | Ac-W-T-I-C(3)-N-N-W-H-D-C(3)-(5OH-W)-P-[D-Lys(DOTA)]-NH2 | 267 |
| 294 | Ac-(1Me-W)-T-I-C(3)-N-N-W-H-D-C(3)-W-P-[D-Lys(DOTA)]-NH2 | 268 |
| 295 | Ac-W-T-I-C(3)-N-N-(7-Me-W)-H-D-C(3)-W-P-[D-Lys(DOTA)]-NH2 | 269 |
| 296 | Ac-W-T-I-C(3)-N-N-(7-MeO-W)-H-D-C(3)-W-P-[D-Lys(DOTA)]-NH2 | 270 |
| 297 | Ac-W-T-I-C(3)-N-N-(7-CI-W)-H-D-C(3)-W-P-[D-Lys(DOTA)]-NH2 | 271 |
| 298 | Ac-W-T-I-C(3)-N-N-W-H-D-C(3)-(7-Me-W)-P-[D-Lys(DOTA)]-NH2 | 272 |
| 299 | Ac-W-T-I-C(3)-N-N-W-H-D-C(3)-(7-MeO-W)-P-[D-Lys(DOTA)]-NH2 | 273 |
| 326 | Ac-W-T-I-C(3)-[3-(4-piperidinyl)-Ala]-N-(2Nal)-K(C12)-D-C(3)-W-P-[D- | 274 |
| | Lys(DOTA)]-NH2 | |
| 329 | Ac-W-T-I-C(1)-N-N-(2Nal)-H-D-C(1)-W-P-[L-Lys(DOTA)]-NH2 | 275 |
| 330 | Ac-7MeW-T-I-C(1)-N-N-(2Nal)-H-D-C(1)-W-P-[D-Lys(DOTA)]-NH2 | 276 |
| 344 | Ac-(7MeO-Trp)-T-I-C(3)-N-N-W-H-D-C(3)-W-P-[D-Lys(DOTA)]-CONH2 | 277 |
| 345 | Ac-W-T-I-C(3)-N-N-W-H-D-C(3)-W-NmeL-[D-Lys(DOTA)]-NH2 | 278 |
| 346 | Ac-W-T-K(C12)-C(3)-N-N-W-H-D-C(3)-W-P-[D-Lys(DOTA)]-NH2 | 279 |
| 347 | Ac-W-T-K(C14)-C(3)-N-N-W-H-D-C(3)-W-P-[D-Lys(DOTA)]-NH2 | 280 |
| 348 | Ac-W-T-K(C16)-C(3)-N-N-W-H-D-C(3)-W-P-[D-Lys(DOTA)]-NH2 | 281 |
| 349 | C12OH-W-T-I-C(3)-N-N-W-H-D-C(3)-W-P-[D-Lys(DOTA)]-NH2 | 282 |
| 352 | Ac-W-T-I-C(3)-N-N-(2Nal)-H-D-C(3)-W-[NmeK(DOTA)]-NH2 | 283 |
| 353 | Ac-W-T-I-C(3)-N-N-(2Nal)-H-D-C(3)-W-P-[D-Lys(G-S-G-S-G-S-DOTA)]- | 284 |
| | NH2 | |
| 354 | Ac-W-T-I-C(3)-N-N-(2Nal)-H-D-C(3)-W-P-[D-Lys(gE-DOTA)]-NH2 | 285 |
| 355 | Ac-W-T-I-C(3)-N-N-(2Nal)-H-D-C(3)-W-P-[D-Lys(gE-gE-gE-DOTA)]-NH2 | 286 |
| 356 | Ac-W-T-I-C(3)-N-N-(2Nal)-H-D-C(3)-W-P-[D-Lys((D-Pro)-PEG8-DOTA)]- | 287 |
| | NH2 | |
| 357 | Ac-W-T-I-C(3)-N-N-(2Nal)-H-D-C(3)-W-P-(D-Pro)3-PEG8-[D-Lys(DOTA)]- | 288 |
| | NH2 | |
| 358 | Ac-W-T-I-C(3)-N-N-(2Nal)-H-D-C(3)-W-P-[D-Lys((D-Pro)3-PEG8-DOTA)]- | 289 |
| | NH2 | |
| 359 | Ac-W-T-I-C(3)-N-N-(2Nal)-H-D-C(3)-W-R-3Me-Aze-[D-Lys(DOTA)]-NH2 | 290 |
| 360 | Ac-W-T-I-C(3)-N-N-(2Nal)-H-D-C(3)-W-aMe-Aze-[D-Lys(DOTA)]-NH2 | 291 |
| 361 | Ac-W-T-I-C(3)-N-N-(2Nal)-H-D-C(3)-W-ACI-[D-Lys(DOTA)]-NH2 | 292 |
| 362 | Ac-W-T-I-C(3)-N-N-(2Nal)-H-D-C(3)-W-3Me2-Aze-[D-Lys(DOTA)]-NH2 | 293 |
| 363 | Ac-W-T-I-C(3)-N-N-(2Nal)-H-D-C(3)-W-P-[D-Lys(SP6-PEG8-DOTA)]-NH2 | 294 |
| 364 | Ac-W-T-I-C(3)-N-N-(2Nal)-H-D-C(3)-W-P-SP6-PEG8-[D-Lys(DOTA)]-NH2 | 295 |
| 365 | Ac-W-T-I-C(3)-N-N-(2Nal)-H-D-C(3)-W-P-[D-Lys(G-G-S-G-G-S-DOTA)]- | 296 |
| | NH2 | |
| 366 | Ac-W-T-I-C(3)-N-N-(2Nal)-(3Pya)-D-C(3)-W-P-[D-Lys(DOTA)]-NH2 | 297 |
| 367 | Ac-W-T-I-C(3)-N-N-(2Nal)-(4Pya)-D-C(3)-W-P-[D-Lys(DOTA)]-NH2 | 298 |
| 368 | Ac-W-T-I-C(3)-N-N-(2Nal)-K(C12)-D-C(3)-W-P-[D-Lys(gE-gE-gE-DOTA)]- | 299 |
| | NH2 | |
| 369 | Ac-(7Me-W)-T-I-C(3)-N-N-(2Nal)-H-D-C(3)-W-P-[D-Lys(DOTA)]-NH2 | 300 |
| 371 | Ac-W-T-I-C(3)-N-N-(2Nal)-H-D-C(3)-W-P-(Sar)5-[D-Lys(DOTA)]-NH2 | 301 |
| 372 | Ac-W-T-I-C(3)-N-N-(2Nal)-H-D-C(3)-W-P-(Ahx)2-[D-Lys(DOTA)]-NH2 | 302 |
| 373 | Ac-W-T-I-C(3)-N-N-(2Nal)-H-D-C(3)-W-P-(Cyx)2-[D-Lys(DOTA)]-NH2 | 303 |
| 374 | Ac-W-T-I-C(3)-N-N-(2Nal)-H-D-C(3)-W-P-(AMBX)-[D-Lys(DOTA)]-NH2 | 304 |
| 375 | Ac-W-T-I-C(3)-N-N-(2Nal)-Arg-D-C(3)-W-P-[D-Lys(DOTA)]-NH2 | 305 |
| 376 | Ac-W-T-I-C(3)-N-N-(2Nal)-Orn-D-C(3)-W-P-[D-Lys(DOTA)]-NH2 | 306 |
| 409 | Ac-W-T-(aMe-I)-C(3)-N-N-W-H-D-C(3)-W-P-[D-Lys(DOTA)]-NH2 | 307 |
| 410 | Ac-W-T-I-C(3)-N-N-W-H-(aMe-D)-C(3)-W-P-[D-Lys(DOTA)]-NH2 | 308 |
| 411 | Ac-W-T-I-C(3)-Ser-N-W-Ser-D-C(3)-W-P-[D-Lys(DOTA)]-NH2 | 309 |
| 412 | Ac-W-T-I-C(3)-Ser-Ser-W-Ser-D-C(3)-W-P-[D-Lys(DOTA)]-NH2 | 310 |
| 413 | Ac-W-T-I-C(3)-N-Ser-W-Ser-D-C(3)-W-P-[D-Lys(DOTA)]-NH2 | 311 |
| 414 | Ac-W-T-I-C(3)-Ser-N-W-H-D-C(3)-W-P-[D-Lys(DOTA)]-NH2 | 312 |
| 415 | Ac-W-T-I-C(3)-Ser-Ser-W-H-D-C(3)-W-P-[D-Lys(DOTA)]-NH2 | 313 |
| 426 | Ac-WT-(Chg)-C(3)-[3-(4-piperidinyl)-Ala]-N-(2Nal)-Lys(PEG24- | 314 |
| | [gE(C16)]-OH)-D-C(3)-W-P-[D-Lys(DOTA)]-NH2 | |
| 427 | Ac-W-T-(NMe-Nle)-C(3)-[3-(4-piperidinyl)-Ala]-N-(2Nal)-Lys(PEG24- | 315 |
| | [gE(C16)]-OH)-D-C(3)-W-P-[D-Lys(DOTA)]-NH2 | |
| 451 | Ac-W-T-I-C(3)-K-N-(2Nal)-H-D-C(3)-W-P-[D-Lys(DOTA)]-CONH2 | 316 |

or a pharmaceutically acceptable salt thereof.

Note: C(3) and/or Pen(3) in the above sequences in Table B and below indicates the two Cysteine residues involved in cyclic bond formation with disulfide.

Note: C(1) and/or D-Cys(1) in the above sequences in Table B and below indicates the two Cysteine residues involved in cyclic bond formation with a thioacetal bridge (S—CH₂—S or methylene cross-linker).

In another aspect, provided herein is a pharmaceutical composition comprising a peptide described herein, or a pharmaceutically acceptable salt thereof, and a pharmaceutically acceptable carrier.

The compounds disclosed herein may exist as tautomers and optical isomers (e.g., enantiomers, diastereomers, diastereomeric mixtures, racemic mixtures, and the like). The absolute stereochemistry is specified according to the Cahn-Ingold-Prelog R—S system. Chiral centers, of which the absolute configurations are known, are labelled by prefixes R and S, assigned by the standard sequence-rule procedure, and preceded when necessary, by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either I or (S), and a compound designated as (S*) refers to a compound that is a pure single isomer at that stereocenter with an absolute configuration of either(R) or (S).

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Examples of bridging moieties/peptide staples for use with compounds of the present disclosure include, but are not limited to: amide-based (e.g., lactam) bridges; aromatic-ring-based bridges; hydrocarbon chains; alkene-based hydrocarbon bridges (e.g., using Fmoc-′-2-(2′-pentenyl)alanine); triazole-based Click bridges, such as copper(I)-catalyzed Huisgen 1,3-dipolar cycloaddition reactions between side chain azido and alkynyl moieties (e.g., Fmoc-L-Nle(εN3) and Fmoc-D-Pra) (see S. Kawamoto, et al., J. Med. Chem. 2012, 55(3), 1137-1146); dialkynyl staples (e.g., 1,4-diethynylbenzene, diethynylpentane, diethynylamines) for stapling linear diazido-peptides; sulfide-bonded disulfide, thioether, and bis-thioether bridges; perfluorobenzene bridges; or combinations thereof.

In some embodiments, bridging moieties comprise an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue. In some embodiments, the bridging moiety comprises an amide bond produced by the reaction of the side chains of the following pairs of amino acids: lysine and glutamate; lysine and aspartate; ornithine and glutamate; ornithine and aspartate; homolysine and glutamic acid; homolysine and aspartic acid; and other combinations of amino acids, unnatural amino acids or non-amino acid residues comprising a primary amine and a carboxylic acid. In some embodiments, bridging moieties are formed through cyclization reactions using olefin metathesis.

In some embodiments, the bridging moiety comprises a disulfide bond formed between two thiol containing residues. In some embodiments, the bridging moiety comprises one or more thioether bonds. Such thioether bonds may include those found in cyclo-thioalkyl compounds. These bonds can be formed during a chemical cyclization reaction between chloro acetic acid N-terminal modified groups and cysteine residues. In some embodiments, bridging moieties comprise one or more triazole ring.

In some embodiments, bridging moieties comprise one or more hydrocarbon chains (linear or branched), and/or hydrocarbon rings (cyclic, heterocyclic, aromatic, heteroaromatic). In some embodiments, hydrocarbon bridging moieties may be introduced by reaction with reagents containing multiple reactive halides, including, but not limited to poly(bromomethyl)benzenes, poly(bromomethyl)pyridines, poly(bromomethyl)alkyl benzenes alor (E)-1,4-dibromobut-2-ene. Examples of Poly(bromomethyl)benzene molecules of the present disclosure can include 1,2-bis(bromomethyl)benzene; 1,3-bis(bromomethyl)benzene; and 1,4-bis(bromomethyl)benzene.

In some embodiments, the thiol group of a cysteine residue is cross-linked with another cysteine residue to form a disulfide bond. In some embodiments, thiol groups of cysteine residues react with bromomethyl groups of poly(bromomethyl)benzene molecules to form stable linkages (see, e.g., Timmerman et al., Chem Bio Chem (2005) 6:821-824, the contents of which are incorporated herein by reference in their entirety).

In some embodiments, Bis-, tris- and tetrakis(bromomethyl)benzene molecules can be used to generate bridging moieties to produce peptides with one, two or three loops, respectively. Bromomethyl groups of a poly(bromomethyl)benzene molecule may be arranged on the benzene ring on adjacent ring carbons (ortho- or o-), with a ring carbon separating the two groups (meta- or m-) or on opposite ring carbons (para- or p-). In some embodiments, m-bis(bromomethyl)benzene (i.e., m-dibromoxylene), o-bis(bromomethyl)benzene (i.e., o-dibromoxylene) and/or p-bis(bromomethyl)benzene (i.e., p-dibromoxylene) are used to form cyclic peptides. In some embodiments, thiol groups of cysteine residues react with other reagents comprising one or more bromo functional groups to form stable linkages. Such reagents may include, but are not limited to poly(bromomethyl) pyridines (e.g., 2,6-bis(bromomethyl) pyridine), poly(bromomethyl)alkyl benzenes (e.g., 1,2-bis(bromomethyl)-4-alkylbenzeneInd/or (E)-1,4-dibromobut-2-ene.

In some embodiments, a side chain amino group and a terminal amino group are cross-linked with disuccinimidyl glutarate (see, e.g., Millward et al., J. Am. Chem. Soc. (2005) 127:14142-14143. In some embodiments, an enzymatic method is used which relies on the reaction between (1) a cysteine and (2) a dehydroalanine or dehydrobutyrine group, catalyzed by a lantibiotic synthetase, to create the thioether bond (see, e.g., Levengood et al., Bioorg. and Med. Chem. Lett. (2008) 18:3025-3028). The dehydro functional group can also be generated chemically by the oxidation of selenium containing amino acid side chains incorporated during translation (see, e.g., Seebeck et al., J. Am. Chem. Soc. 2006).

In some embodiments, bridging moieties comprise an aromatic, 6-membered ring (e.g., benzene). In some embodiments, bridging moieties comprise a heterocyclic, 6-membered ring which includes one nitrogen atoms (e.g., pyridine). In some embodiments, bridging moieties comprise a heterocyclic, 6-membered ring which includes two nitrogen atoms (e.g., pyridazine, pyrimidine, pyrazine). In some embodiments, bridging moieties comprise a heterocyclic, 6-membered ring which includes three nitrogen atoms (e.g., triazanes). In some embodiments, bridging moieties comprise a heterocyclic, 5-membered ring which includes one nitrogen atoms (e.g., pyrrole). In some embodiments, bridging moieties comprise a heterocyclic, 5-membered ring which includes two nitrogen atoms (e.g., imidazole, pyrazole). In some embodiments, bridging moieties comprise a heterocyclic, 5-membered ring which includes three nitrogen atoms (e.g., triazoles).

Peptides of the present disclosure may be cyclized through the carboxy terminus, the amino terminus, or through any other convenient point of attachment, such as, for example, through the sulfur of a cysteine (e.g., through the formation of disulfide bonds between two cysteine residues in a sequence) or any side-chain of an amino acid residue. Further linkages forming cyclic loops may include, but are not limited to, maleimide linkages, amide linkages, ester linkages, ether linkages, thiol ether linkages, hydrazone linkages, or acetamide linkages.

In some embodiments, peptides of the disclosure are formed using a lactam moiety. Such cyclic peptides may be formed, for example, by synthesis on a solid support Wang resin using standard Fmoc chemistry. In some cases, Fmoc-ASP(allyl)-OH and Fmoc-LYS(alloc)-OH are incorporated into peptides to serve as precursor monomers for lactam bridge formation.

In some embodiments, peptides of the present disclosure are linear peptides. In some embodiments, peptides of the present disclosure are cyclic peptides. In some embodiments, the cyclic peptides comprise a disulfide bond. In some embodiments, peptides of the present disclosure are linear peptides prior to the cyclization step. In some embodiments, peptides of the present disclosure are linear peptides prior to the formation of a disulfide bond.

Generally, disulfide bond formation involves a reaction between the sulfhydryl (SH) side chains of two cysteine residues. Proper disulfide bonds provide stability to a protein, decreasing further entropic choices that facilitate folding progression toward the native state by limiting unfolded or improperly folded conformations.

### Terminal Modifications and Conjugations

One method of protecting a peptide from proteolytic degradation involves chemical modification or “capping” of the amino and/or carboxy terminus of the peptides. As used herein, the terms “chemically modified” or “capped” are used interchangeably to refer to the introduction of a blocking group at the end or both ends of the compound by covalent modification. Suitable blocking groups serve to block the ends of the peptides without decreasing the biological activity of the peptides. Any residue located at the amino or carboxy terminus, or both of the described compounds can be chemically modified. In some embodiments, peptides of the present disclosure comprise an N-terminal and/or C-terminal modification.

In one embodiment, the amino end of the compound is chemically modified by acetylation to produce an N-acetylated peptide (which may be represented by “Ac-” in the structure or formula of the present disclosure). In another embodiment, the carboxy terminus of the described peptides is chemically modified by amidation to give the primary carboxamide at the C-terminus (which may be represented as “amide” in the peptide sequence, structure or claims of the present disclosure). In some embodiments, both the amino end and the carboxy end are chemically modified by acetylation and amidation, respectively. However, other capping groups are possible. For example, the amino end can be capped by acylation with groups such as an acetyl group, a benzoyl group, or natural or non-natural amino acids, such as beta-alanine, capped by an acetyl group; or by alkylation with groups such as a benzyl group or a butyl group, or by sulfonylation to produce sulfonamides. Similarly, the carboxy terminus can be esterified or converted to a secondary amide and acylsulfonamide or the like.

In some embodiments, the N-terminal capping function is in a linkage to the terminal amino group and may be selected from the group: formyl; alkanoyl, having from 1 to 10 carbon atoms, such as acetyl, propionyl, butyryl; alkenoyl, having from 1 to 10 carbon atoms, such as hex-3-enoyl; alkynoyl, having from 1 to 10 carbon atoms, such as hex-5-ynoyl; aroyl, such as benzoyl or 1-naphthoyl; heteroaroyl, such as 3-pyrroyl or 4-quinoloyl; alkylsulfonyl, such as methanesulfonyl; arylsulfonyl, such as benzenesulfonyl or sulfanilyl; heteroarylsulfonyl, such as pyridine-4-sulfonyl; substituted alkanoyl, having from 1 to 10 carbon atoms, such as 4-aminobutyryl; substituted alkenoyl, having from 1 to 10 carbon atoms, such as 6-hydroxy-hex-3-enoyl; substituted alkynoyl, having from 1 to 10 carbon atoms, such as 3-hydroxy-hex-5-ynoyl; substituted aroyl, such as 4-chlorobenzoyl or 8-hydroxy-naphth-2-oyl; substituted heteroaroyl, such as 2,4-dioxo-1,2,3,4-tetrahydro-3-methyl-quinazolin-6-oyl; substituted alkylsulfonyl, such as 2-aminoethanesulfonyl; substituted arylsulfonyl, such as 5-dimethylamino-1-naphthalenesulfonyl; substituted heteroarylsulfonyl, such as 1-methoxy-6-isoquinolinesulfonyl; carbamoyl or thiocarbamoyl; substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl; substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined; Lys-(Gly)n, where n=1-8; or Tyr-(Gly)n where n=1-8.

In some embodiments, the C-terminal capping function can either be in an amide bond with the terminal carboxyl or in an ester bond with the terminal carboxyl. Capping functions that provide for an amide bond are designated as NR1R2 wherein each R1 and R2 may be independently selected from the following group: hydrogen; alkyl, having from 1 to 10 carbon atoms, such as methyl, ethyl, isopropyl; alkenyl, preferably having from 1 to 10 carbon atoms, such as prop-2-enyl; alkynyl, preferably having from 1 to 10 carbon atoms, such as prop-2-ynyl; substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl; substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl; substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl; aroylalkyl having up to 10 carbon atoms, such as phenacyl or 2-benzoylethyl; aryl, such as phenyl or 1-naphthyl; heteroaryl, such as 4-quinolyl; alkanoyl having from 1 to 10 carbon atoms, such as acetyl or butyryl; aroyl, such as benzoyl; heteroaroyl, such as 3-quinoloyl; OR′ or NR′R″ where R′ and R″ each are independently hydrogen, alkyl, aryl, heteroaryl, acyl, aroyl, sulfonyl, sulfinyl, SO2-R′″ or SO—R′″ where R′″ is substituted or unsubstituted alkyl, aryl, heteroaryl, alkenyl, or alkynyl.

In some embodiments, capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

In some embodiments, peptides of the present disclosure can comprise modifications to the C-terminus of the peptide sequence with one or more of the following moieties: NH₂, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, or OH.

In some embodiments, peptides of the present disclosure can comprise modifications to the N-terminus of the peptide sequence with one or more peptide-based moieties. In some embodiments, peptides of the present disclosure can comprise modifications to the C-terminus of the peptide sequence with one or more peptide-based moieties. In some embodiments, peptides of the present disclosure can comprise modifications to both the N-terminus of the peptide sequence and the C-terminus of the peptide sequence with one or more peptide-based moieties.

In some embodiments, peptides of the present disclosure can comprise modifications to the N-terminus of the peptide sequence with one or more non-peptide-based moieties. In some embodiments, peptides of the present disclosure can comprise modifications to the C-terminus of the peptide sequence with one or more non-peptide-based moieties. In some embodiments, peptides of the present disclosure can comprise modifications to both the N-terminus of the peptide sequence and the C-terminus of the peptide sequence with one or more non-peptide-based moieties.

In some embodiments, peptides of the present disclosure can comprise modifications to the N-terminus which comprise a string of 5 or 6 Glu amino acids. In some embodiments, peptides of the present disclosure can comprise modifications to the N-terminus which comprise a string of 5 or 6 Lys amino acids. In some embodiments, peptides of the present disclosure can comprise modifications to the N-terminus which comprise a string of 5 or 6 amino acids, each independently selected from Glu or Lys.

In some embodiments, peptides of the present disclosure comprise an N-terminal peptide consisting of a chain of about 15 to about 400 identical amino acids. In some embodiments, the N-terminal peptide comprises about 25 to about 300 identical amino acids, about 50 to about 200 identical amino acids, about 75 to about 150 identical amino acids, about 90 to about 120 identical amino acids, or about 100 or 110 identical amino acids. In some embodiments, the N-terminal peptide comprises: poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof.

In some embodiments, peptides of the present disclosure comprise an N-terminal modification. In a further embodiment, the N-terminal modification comprises an N-terminal acetyl group (represented as Ac). For example, in SEQ ID NO: 24-44 and 67-89 in Table 2, the N-terminal methionine group is capped with acetic anhydride or other appropriate reagents during peptide synthesis leading to a molecule which is N-terminally acetylated. In some embodiments, peptides of the present disclosure comprise a C-terminal modification. In a further embodiment, the C-terminal modification comprises an amide group (represented as amide or CONH2). For example, in SEQ ID NO: 24-44 and 67-89 in Table 2, the C-terminal serine group is synthesized as an amide during peptide synthesis leading to a molecule which is C-terminally amidated.

In some embodiments, targeting moieties include one or more peptide sequences binding to DLL3 listed in Table 2 or a fragment or variant thereof. In some embodiments, targeting constructs include targeting moieties that include one or more peptide sequences binding to DLL3 listed in Table 2 or a fragment or variant thereof.

For example, amino acid sequences with SEQ ID NO: 3-24 are unmodified linear peptides without capping groups prior to formation a disulfide bond. Amino acid sequences with SEQ ID NO: 24-44 are linear peptides with acetyl group at the N-terminus and amide group at the C-terminus prior to formation a disulfide bond. Amino acid sequences with SEQ ID NO: 45-66 are unmodified cyclic peptides without capping groups after formation a disulfide bond. Amino acid sequences with SEQ ID NO: 67-89 are cyclic peptides with acetyl group at the N-terminus and amide group at the C-terminus after formation a disulfide bond.

TABLE 2
Peptide sequences binding to DLL3

| | SEQ |
|---|---|
| | ID |
| Sequence | NO |
| MWTICADWAQCWP | 3 |
| WTICADWAQCWP | 4 |
| MWTICNNWHDCWP | 5 |
| MWKICSDWQNCWP | 6 |
| MWHICQEWATCWP | 7 |
| MWTLCDTWDTCFP | 8 |
| MWTLCDNWQTCWP | 9 |
| MWQLCEWNNCWPV | 10 |
| MWVLCEWHECWPG | 11 |
| MDCNFFDCDAWTW | 12 |
| MWMLCDWDKDCWP | 13 |
| MVWVPCEWDDCFY | 14 |
| MSDCWLQWNCPFD | 15 |
| WTICNNWHDCWP | 16 |
| AWTICNNWHDCWP | 17 |
| MWTICNNWHACWP | 18 |
| MWTICNNWHDCAP | 19 |
| MWTICNNWHDCWA | 20 |
| MWAICNNWHDCWP | 21 |
| MWTACNNWHDCWP | 22 |
| MWTICANWHDCWP | 23 |
| Ac-MWTICADWAQCWP-CONH2 | 24 |
| Ac-WTICADWAQCWP-CONH2 | 25 |
| Ac-MWTICNNWHDCWP-CONH2 | 26 |
| Ac-MWKICSDWQNCWP-CONH2 | 27 |
| Ac-MWHICQEWATCWP-CONH2 | 28 |
| Ac-MWTLCDTWDTCFP-CONH2 | 29 |
| Ac-MWTLCDNWQTCWP-CONH2 | 30 |
| Ac-MWQLCEWNNCWPV-CONH2 | 31 |
| Ac-MWVLCEWHECWPG-CONH2 | 32 |
| Ac-MDCNFFDCDAWTW-CONH2 | 33 |
| Ac-MWMLCDWDKDCWP-CONH2 | 34 |
| Ac-MVWVPCEWDDCFY-CONH2 | 35 |
| Ac-MSDCWLQWNCPFD-CONH2 | 36 |
| Ac-WTICNNWHDCWP-CONH2 | 37 |
| Ac-AWTICNNWHDCWP-CONH2 | 38 |
| Ac-MWTICANWHDCWP-CONH2 | 39 |
| Ac-MWTICNNWHACWP-CONH2 | 40 |
| Ac-MWTICNNWHDCAP-CONH2 | 41 |
| Ac-MWTICNNWHDCWA-CONH2 | 42 |
| Ac-MWAICNNWHDCWP-CONH2 | 43 |
| Ac-MWTACNNWHDCWP-CONH2 | 44 |
| [Cyc(5, 11)]MWTICADWAQCWP | 45 |
| [Cyc(4, 10)]WTICADWAQCWP | 46 |
| [Cyc(5,11)]MWTICNNWHDCWP | 47 |
| [Cyc(5,11)]MWKICSDWQNCWP | 48 |
| [Cyc(5, 11)]MWHICQEWATCWP | 49 |
| [Cyc(5,11)]MWTLCDTWDTCFP | 50 |
| [Cyc(5,11)]MWTLCDNWQTCWP | 51 |
| [Cyc(5,10)]MWQLCEWNNCWPV | 52 |
| [Cyc(5, 10)]MWVLCEWHECWPG | 53 |
| [Cyc(3,8)]MDCNFFDCDAWTW | 54 |
| [Cyc(5,11)]MWMLCDWDKDCWP | 55 |
| [Cyc(6, 11)]MVWVPCEWDDCFY | 56 |
| [Cyc(4, 10)]MSDCWLQWNCPFD | 57 |
| [Cyc(4, 10)]WTICNNWHDCWP | 58 |
| [Cyc(5, 11)]AWTICNNWHDCWP | 59 |
| [Cyc(5, 11)]MWTICANWHDCWP | 60 |
| [Cyc(5,11)]MWTICNNWHACWP | 61 |
| [Cyc(5,11)]MWTICNNWHDCAP | 62 |
| [Cyc(5,11)]MWTICNNWHDCWA | 63 |
| [Cyc(5, 11)]MWAICNNWHDCWP | 65 |
| [Cyc(5,11)]MWTACNNWHDCWP | 66 |
| [Cyc(5, 11)]Ac-MWTICADWAQCWP-CONH2 | 67 |
| [Cyc(4, 10)]Ac-WTICADWAQCWP-CONH2 | 68 |
| [Cyc(5, 11)]Ac-MWTICNNWHDCWP-CONH2 | 69 |
| [Cyc(5, 11)]Ac-MWKICSDWQNCWP-CONH2 | 70 |
| [Cyc(5, 11)]Ac-MWHICQEWATCWP-CONH2 | 71 |
| [Cyc(5, 11)]Ac-MWTLCDTWDTCFP-CONH2 | 72 |
| [Cyc(5, 11)]Ac-MWTLCDNWQTCWP-CONH2 | 73 |
| [Cyc(5, 10)]Ac-MWQLCEWNNCWPV-CONH2 | 74 |
| [Cyc(5, 10)]Ac-MWVLCEWHECWPG-CONH2 | 75 |
| [Cyc(3,8)]Ac-MDCNFFDCDAWTW-CONH2 | 76 |
| [Cyc(5, 11)]Ac-MWMLCDWDKDCWP-CONH2 | 77 |
| [Cyc(6, 11)]Ac-MVWVPCEWDDCFY-CONH2 | 78 |
| [Cyc(4, 10)]Ac-MSDCWLQWNCPFD-CONH2 | 79 |
| [Cyc(4, 10)]DOTA-WTICNNWHDCWP-CONH2 | 80 |
| [Cyc(5, 11)]Ac-(d-Ala)WTICNNWHDCWP-CONH2 | 81 |
| [Cyc(5, 11)]Ac-MWTIC(d-Ala)NWHDCWP-CONH2 | 82 |
| [Cyc(5, 11)]Ac-MWTICNNWH(d-Ala)CWP-CONH2 | 83 |
| [Cyc(5, 11)]Ac-MWTICNNWHDC(d-Ala)P-CONH2 | 84 |
| [Cyc(5, 11)]Ac-MWTICNNWHDCW(d-Ala)-CONH2 | 85 |
| [Cyc(5, 11)]Ac-(L-Ala)WTICNNWHDCWP-CONH2 | 86 |
| [Cyc(5, 11)]Ac-MW(L-Ala)ICNNWHDCWP-CONH2 | 87 |
| [Cyc(5, 11)]Ac-MWT(L-Ala)CNNWHDCWP-CONH2 | 88 |
| [Cyc(5, 11)]Ac-MWTIC(L-Ala)NWHDCWP-CONH2 | 89 |

Polypeptides of the disclosure may be peptidomimetics. A “peptidomimetic” or “polypeptide mimetic” is a polypeptide in which the molecule contains structural elements that are not found in natural polypeptides (i.e., polypeptides comprised of only the 20 proteinogenic amino acids). In some embodiments, peptidomimetics are capable of recapitulating or mimicking the biological action(s) of a natural peptide. A peptidomimetic may differ in many ways from natural polypeptides, for example through changes in backbone structure or through the presence of amino acids that do not occur in nature. In some cases, peptidomimetics may include amino acids with side chains that are not found among the known 20 proteinogenic amino acids; non-polypeptide-based bridging moieties used to effect cyclization between the ends or internal portions of the molecule; substitutions of the amide bond hydrogen moiety by methyl groups (N-methylation) or other alkyl groups; substitutions of the amino acid alpha hydrogen moiety by methyl groups (alpha-methylation) or other alkyl groups; replacement of a peptide bond with a chemical group or bond that is resistant to chemical or enzymatic treatments; N- and C-terminal modifications; and/or conjugation with a non-peptidic extension (such as polyethylene glycol, lipids, carbohydrates, nucleosides, nucleotides, nucleoside bases, various small molecules, or phosphate or sulfate groups).

As used herein, the term “amino acid” includes the residues of the natural amino acids as well as unnatural amino acids. The 20 natural proteinogenic amino acids are identified and referred to herein by either the one-letter or three-letter designations as follows: aspartic acid (Asp:D), isoleucine (Ile:1), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagine (Asn:N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g., acetyl or benzyloxycarbonyl), as well as natural and unnatural amino acids protected at the carboxy terminus (e.g., as a (C1-C6) alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc., and documents cited therein, the contents of each of which are herein incorporated by reference in their entirety). Peptides and/or peptide compositions of the present disclosure may also include modified amino acids.

“Unnatural” amino acids have side chains or other features not present in the 20 naturally-occurring amino acids listed above and include, but are not limited to: N-methyl amino acids, N-alkyl amino acids, alpha, alpha substituted amino acids, beta-amino acids, alpha-hydroxy amino acids, D-amino acids, and other unnatural amino acids known in the art (See, e.g., Josephson et al., (2005) J. Am. Chem. Soc. 127: 11727-11735; Forster, A. C. et al. (2003) Proc. Natl. Acad. Sci. USA 100: 6353-6357; Subtelny et al., (2008) J. Am. Chem. Soc. 130: 6131-6136; Hartman, M. C. T. et al. (2007) PLoS ONE 2:e972; and Hartman et al., (2006) Proc. Natl. Acad. Sci. USA 103:4356-4361). Further unnatural amino acids useful for the optimization of peptides and/or peptide compositions of the present disclosure include, but are not limited to 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-afminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine (also known as tert-leucine), phenylglycine, azatryptophan, 5-azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methylphenylglycine, 4-ethyl-phenylglycine, 4-isopropyl-phenylglycine, (S)-2-amino-5-azidopentanoic acid (also referred to herein as “X02”), (S)-2-aminohept-6-enoic acid (also referred to herein as “X30”), (S)-2-aminopent-4-ynoic acid (also referred to herein as “X31”), (S)-2-aminopent-4-enoic acid (also referred to herein as “X12”), (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-terleucinol, (R)-3-methylbutan-2-amine, (S)-2-methyl-1-phenylpropan-1-amine, and (S)-N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid, (S)-2-amino-3-(oxazol-2-yl)butanoic acid, (S)-2-amino-3-(oxazol-5-yl) butanoic acid, (S)-2-amino-3-(1,3,4-oxadiazol-2-yl) butanoic acid, (S)-2-amino-3-(1,2,4-oxadiazol-3-yl) butanoic acid, (S)-2-amino-3-(5-fluoro-1H-indazol-3-yl) butanoic acid, and (S)-2-amino-3-(1H-indazol-3-yl) butanoic acid, 2-(2′MeOphenyl)-2-amino acetic acid, tetrahydro 3-isoquinolinecarboxylic acid and stereoisomers thereof (including, but not limited, to D and L isomers).

Additional unnatural amino acids that are useful in the optimization of peptides or peptide compositions of the disclosure include but are not limited to halogenated amino acids wherein one or more carbon bound hydrogen atoms are replaced by one or more halogen atoms. The number of halogen atoms included can range from 1 up to and including all of the hydrogen atoms.

In some embodiments, unnatural amino acids that are useful in the optimization of peptides or peptide compositions of the disclosure include but are not limited to fluorinated amino acids wherein one or more carbon bound hydrogen atoms are replaced by one or more fluorine atoms. The number of fluorine atoms included can range from 1 up to and including all of the hydrogen atoms. Examples of such amino acids include but are not limited to 3-fluoroproline, 3,3-difluoroproline, 4-fluoroproline, 4,4-difluoroproline, 3,4-difluroproline, 3,3,4,4-tetrafluoroproline, 4-fluorotryptophan, 5-flurotryptophan, 6-fluorotryptophan, 7-fluorotryptophan, and stereoisomers thereof.

In some embodiments, unnatural amino acids that are useful in the optimization of peptides or peptide compositions of the disclosure include but are not limited to chlorinated amino acids wherein one or more carbon bound hydrogen atoms are replaced by one or more chlorine atoms. The number of chlorine atoms included can range from 1 up to and including all of the hydrogen atoms.

Further unnatural amino acids that are useful in the optimization of peptides of the disclosure include but are not limited to those that are disubstituted at the α-carbon. These include amino acids in which the two substituents on the α-carbon are the same, for example α-amino isobutyric acid, and 2-amino-2-ethyl butanoic acid, as well as those where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidinnne-4-carboxylic acid, and stereoisomers thereof.

Additional unnatural amino acids that are useful in the optimization of peptides or peptide compositions of the disclosure include but are not limited to analogs of tryptophan in which the indole ring system is replaced by another 9 or 10 membered bicyclic ring system comprising 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent may be independently selected from H, F, Cl, Br, CN, COOR, CONRR′, oxo, OR, NRR′. Each R and R′ may be independently selected from H, C1-C20 alkyl, or C1-C20 alkyl-O—C1-20 alkyl.

In some embodiments, analogs of tryptophan (also referred to herein as “tryptophan analogs”) may be useful in the optimization of peptides or peptide compositions of the disclosure. Tryptophan analogs may include, but are not limited to 5-fluorotryptophan [(5-F)W], 5-methyl-O-tryptophan [(5-MeO)W], 1-methyltryptophan [(1-Me-W) or (1-Me)W], D-tryptophan (D-Trp), azatryptophan (including, but not limited to 4-azatryptophan, 7-azatryptophan and 5-azatryptophan) 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

Modified amino acid residues useful for the optimization of peptides and/or peptide compositions of the present disclosure include, but are not limited to those which are chemically blocked (reversibly or irreversibly); chemically modified on their N-terminal amino group or their side chain groups; chemically modified in the amide backbone, as for example, N-methylated, D (unnatural amino acids) and L (natural amino acids) stereoisomers; or residues wherein the side chain functional groups are chemically modified to another functional group. In some embodiments, modified amino acids include without limitation, methionine sulfoxide; methionine sulfone; aspartic acid-(beta-methyl ester), a modified amino acid of aspartic acid; N-ethylglycine, a modified amino acid of glycine; alanine carboxamide; and/or a modified amino acid of alanine. Unnatural amino acids may be purchased from Sigma-Aldrich (St. Louis, MO), Bachem (Torrance, CA) or other suppliers. Unnatural amino acids may further include any of those listed in Table 2 of US patent publication US 2011/0172126, the content of which is incorporated herein by reference in its entirety.

In some embodiments, amino acids for use in the present disclosure are modified using an organic proteinaceous or non-proteinaceous derivatizing agent. In some embodiments, amino acids for use in the present disclosure are modified using post-translational modification. In some embodiments, modifications are introduced by reacting targeted amino acid residues of the peptide with an organic derivatizing agent that is capable of reacting with selected side-chains or terminal residues. In some embodiments, modifications are introduced by harnessing mechanisms of post-translational modifications that function in selected recombinant host cells. Certain post-translational modifications are the result of the action of recombinant host cells on an expressed peptide. As one examples, glutaminyl and asparaginyl residues are frequently post-translationally deamidated to the corresponding glutamyl and aspartyl residues under certain post-translational conditions (e.g., under mildly acidic conditions). Other post-translational modifications include: hydroxylation of proline and lysine; phosphorylation of hydroxyl groups of tyrosinyl, seryl or threonyl residues; and methylation of the alpha-amino groups of lysine, arginine, and histidine side chains (see, e.g., Creighton et al., Proteins: Structure and Molecular Properties, W.H. Freeman & Co., San Francisco, 1983, pp. 79-86).

In some embodiments, amino acid modifications include the bonding of non-proteinaceous polymers to peptides of the present disclosure. Examples of non-proteinaceous polymers include hydrophilic synthetic polymers (i.e., non-natural polymers), such as hydrophilic polyvinyl polymers (e.g., polyvinylalcohol and polyvinylpyrrolidone). The Examples of non-proteinaceous polymers also include polyethylene glycol, polypropylene glycol and polyoxyalkylenes. In some embodiments, amino acid modifications include the bonding of non-proteinaceous polymers to peptides of the present disclosure, as described in U.S. Pat. Nos. 4,640,835, 4,496,689, 4,301,144, 4,670,417, 4,791,192, and 4,179,337; the contents of which are each incorporated herein by reference in their entireties, as related to amino acid modifications for us in the present disclosure.

### Synthesis of Peptides

The present disclosure presents methods of synthesizing peptides and compounds of the present disclosure. In some embodiments, peptides of the present disclosure can be obtained by inducing the formation of a covalent bond between an amino group at the N-terminus of a peptide (if provided), and a carboxyl group of a reactive amino acid side chain moiety (if provided). In some embodiments, peptides and compounds of the present disclosure can be synthesized by any known conventional procedure for the formation of a peptide linkage between amino acids. Such conventional procedures include, for example, any solution phase procedure permitting a condensation between the free alpha amino group of an amino acid or residue thereof (having its carboxyl group or other reactive groups protected) and the free primary carboxyl group of another amino acid or residue thereof (having its amino group or other reactive groups protected). In some embodiments, the peptides of the present disclosure may be synthesized by solid-phase synthesis and purified according to methods known in the art. Any of a number of well-known procedures utilizing a variety of resins and reagents may be used to prepare the peptides of the present disclosure.

In some embodiments, the process for synthesizing peptides may be carried out by a procedure whereby each amino acid in the desired sequence is added one at a time in succession to another amino acid or residue thereof. In some embodiments, the process for synthesizing peptides may be carried out by a procedure whereby multiple peptide fragments with portions of the desired amino acid sequence are first synthesized, and then condensed to provide the desired peptide sequence.

In some embodiments, the process for synthesizing peptides may be carried out using solid phase peptide synthesis, which includes methods well known and practiced in the art (e.g., Symphony Multiplex Peptide Synthesizer (Rainin Instrument Company) automated peptide synthesizer). In some embodiments, the process for synthesizing peptides may be carried out using standard Fmoc methodology on an automated synthesizer (e.g., Advanced Chem Tech 440M05, Louisville, Ky). In some embodiments, the process for synthesizing peptides may be carried using coupling reagents such as 2-(1-H-benzotriazol-1-yl)-1,1,3,3-tetramethyluronium hexafluorophosphate (HBTU) and/or 1-hydroxybenzotriazole (HOBt).

Solid phase peptide synthesis can be carried out by sequentially incorporating the desired amino acid residues one at a time into the growing side chain according to the general principles of solid phase methods. These methods are disclosed in numerous references, including Merrifield, et al., Solid phase synthesis (Nobel lecture), Angew. Chem. (1985) 24:799-810; Barany et al., The Peptides, Analysis, Synthesis and Biology, Vol. 2; Gross et al., Eds. Academic Press 1-284 (1980), the contents of which are each incorporated herein by reference in their entirety, as related to processes and protocols for synthesizing peptides.

Solid phase synthesis of the peptide is generally commenced from the C-terminal end of the peptide by coupling a protected alpha amino acid to a suitable resin. Examples of known methods for preparing substituted amide derivatives on solid-phase have been described in the art (see, e.g., Barn D. R. et al., Tetrahedron Letters (1996), 37:3213-3216; DeGrado et al., J. Org. Chem., (1982) 47:3258-3261; the contents of which are each incorporated herein by reference in their entireties as related to methods and systems for solid-phased peptide synthesis). As an example, starting materials can be prepared by attaching an alpha amino-protected amino acid by an ester linkage to a p-benzyloxybenzyl alcohol (Wang) resin or an oxime resin by well-known means. The peptide chain is grown with the desired sequence of amino acids, and the peptide-resin is then treated with a solution of appropriate amine (such as methylamine, dimethylamine, ethylamine, and so on). Peptides employing a p-benzyloxybenzyl alcohol (Wang) resin may be cleaved from the resin by aluminum chloride in DCM, and peptides employing an oxime resin may be cleaved by DCM.

In some embodiments, reactive side chain groups of the various amino acid residues are protected with suitable protecting groups, which prevent a chemical reaction from occurring at that site until the protecting group is removed. In some embodiments, the alpha amino group of an amino acid residue or fragment is protected while that entity reacts at the carboxyl group, followed by the selective removal of the alpha amino protecting group to allow a subsequent reaction to take place at that site. Examples of protecting groups for use in the present disclosure have been disclosed and are known in solid phase synthesis methods and solution phase synthesis methods.

In some embodiments, alpha amino groups may be protected by a suitable protecting group, including: a urethane-type protecting group, such as benzyloxycarbonyl (Cbz or Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl.

In some embodiments, guanidino amino groups (such as those found in arginine) may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf), and Boc.

As a non-limiting example, solid phase synthesis of a peptide can be commenced from the C-terminal end of the peptide by coupling a protected alpha amino acid to a suitable resin. The starting material can be prepared by attaching an alpha amino-protected amino acid by an ester linkage to a p-benzyloxybenzyl alcohol (Wang) resin, a 2-chlorotrityl chloride resin or an oxime resin, by an amide bond between an Fmoc-Linker, such as p-[(R,S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are then carried through repetitive addition cycles as necessary to add amino acids sequentially. The alpha amino Fmoc protecting groups are then removed under basic conditions (e.g., Piperidine, piperazine, diethylamine, or morpholine (20-40% v/v) in N,N-dimethylformamide (DMF)). Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Similarly, methods for modifying side chains of amino acids are well known to those skilled in the art of peptide synthesis. The choice of modifications made to reactive groups present on the peptide will be determined, in part, by the characteristics that are desired in the peptide.

In some embodiments, the N-terminus group is modified by introduction of an N-acetyl group. As a non-limiting example, the peptide synthesis can include a step wherein, after removal of the protecting group at the N-terminal, a resin-bound peptide is reacted with acetic anhydride in dichloromethane in the presence of an organic base, such as diisopropylethylamine. Other methods of N-terminus acetylation are known in the art, including solution phase acetylation.

In some embodiments, peptides of the present disclosure can comprise cyclic peptides having one or more bridging moieties (e.g., cyclic structure, staple, bridge, etc.).

In some embodiments, the peptide can be synthesized using solid phase peptide synthesis, and then cyclized prior to cleavage from the peptide resin. If the peptide is being cyclized through reactive side chain moieties, the desired side chains are first deprotected under specific deprotection conditions in a suitable solvent, and a cyclic coupling agent is then added. Suitable solvents include, but are not limited to: DMF, dichloromethane (DCM), and 1-methyl-2-pyrrolidone (NMP). Suitable cyclic coupling reagents include, but are not limited to: 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1(2H)-pyridyl)-1,1,3,3-tetramethyluronium tetrafluoroborate (TPTU), and N,N′-dicyclohexylcarbodiimide/1-hydroxybenzotriazole (DCCI/HOBt). In some embodiments, coupling of the cyclic moiety to the peptide chain is initiated by use of a suitable base, such as N,N-diisopropylethylamine (DIPEA), sym-collidine, or N-methylmorpholine (NMM).

The cyclized peptides can then be cleaved from the solid phase using any suitable reagent, such as ethylamine in DCM. The resulting crude peptide is dried and remaining amino acid side chain protecting groups (if any) are cleaved using suitable reagents, such as trifluoroacetic acid (TFA) in the presence of water and 1,2-ethanedithiol (EDT). The final product is precipitated by adding cold ether and collected by filtration. Final purification can be by reverse phase high performance liquid chromatography (RP-HPLC), using a suitable column, such as a C18 column. Other methods of separation or purification, such as methods based on the size or charge of the peptide, can also be employed. Once purified, the peptide can be characterized by any number of methods, such as high-performance liquid chromatography (HPLC), amino acid analysis, mass spectrometry, and the like.

In some embodiments, peptides of the present disclosure can comprise one or more modifications (e.g., substitution, addition, or deletion) to one or more terminus (e.g., N-terminus, C-terminus, or both) of the peptide sequence. In some embodiments, terminus-modified peptides can be synthesized using solid phase peptide synthesis, and then modified prior to cleavage from the peptide resin.

The present disclosure contemplates variants and derivatives of peptides presented herein. These include substitutional, insertional, deletional, and covalent variants and derivatives. As used herein, the term “derivative” is used synonymously with the term “variant” and refers to a molecule that has been modified or changed in any way relative to a reference molecule or starting molecule.

In one embodiment, the peptides described herein comprise replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In some embodiments, peptides of the present disclosure may be in the salt forms. The salts of the peptides can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g., L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g., D-glucuronic), glutamic (e.g., L-glutamic), α-oxoglutaric, glycolic, hippuric, hydrohalic acids (e.g., hydrobromic, hydrochloric, hydriodic), isethionic, lactic (e.g., (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic, and valeric acids, as well as acylated amino acids and cation exchange resins.

In some embodiments, salts of the present disclosure may be salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic, and lactobionic acids. In some embodiments, the salt may be the hydrochloride salt. In some embodiments, the salt may be the acetate salt.

If the compound is anionic or has a functional group which may be anionic (e.g., —COOH may be —COO—), then a salt may be formed with an organic or inorganic base, generating a suitable cation. Examples of suitable inorganic cations include, but are not limited to, alkali metal ions such as Li⁺, Na⁺, and K⁺, alkaline earth metal cations such as Ca²⁺ and Mg²⁺, and other cations. Examples of suitable organic cations include, but are not limited to, ammonium ion (i.e., NH₄⁺) and substituted ammonium ions (e.g., NH₃R⁺, NH₂R²⁺, NHR³⁺, NR⁴⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)⁴⁺.

Where the peptides contain an amine function, these may form quaternary ammonium salts, for example by reaction with an alkylating agent according to methods well known to the skilled person.

The peptides disclosed herein, may bind to a target receptor with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM, or at least 1,000 nM.

In some embodiments, the peptides disclosed herein, may bind to DLL3 with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM, or at least 1,000 nM.

### Chelating Agents

Chelating agents (CAs), also referred to herein as “chelators,” may include metal chelating agents that associate with metal cargo (e.g., metallic nuclide cargo). Chelating agents may include macromolecular compounds. In some embodiments, chelating agents include acyclic or macrocyclic compounds.

Exemplary chelating agents (also referred to as “Chelator”) are shown below in Table C.

TABLE C

| Name | |
|---|---|
| (Designation) | Structure |
| DOTA | 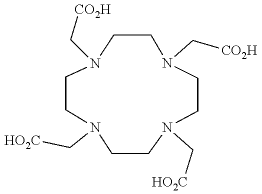 [SMILES: O=C(O)CN1CCN(CC(=O)O)CCN(CC(=O)O)CCN(CC(=O)O)CC1] |
| DO3A | 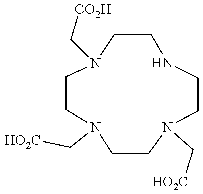 [SMILES: O=C(O)CN1CCNCCN(CC(=O)O)CCN(CC(=O)O)CC1] |
| C-DOTA | 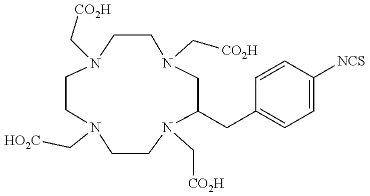 |
| PA-DOTA | 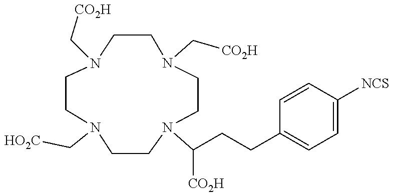 |
| DODASA | 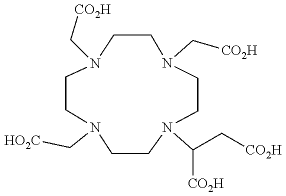 [SMILES: O=C(O)CC(C(=O)O)N1CCN(CC(=O)O)CCN(CC(=O)O)CCN(CC(=O)O)CC1] |
| C4amino- DOTA | 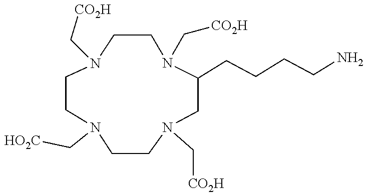 [SMILES: NCCCCC1CN(CC(=O)O)CCN(CC(=O)O)CCN(CC(=O)O)CCN1CC(=O)O] |
| DOTAGA | 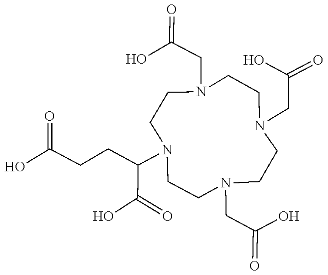 [SMILES: O=C(O)CCC(C(=O)O)N1CCN(CC(=O)O)CCN(CC(=O)O)CCN(CC(=O)O)CC1] |
| DOTASA | 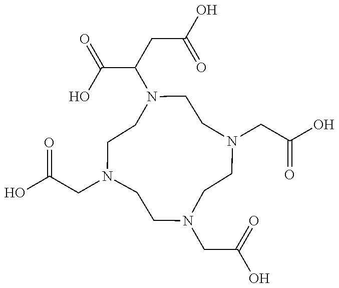 [SMILES: O=C(O)CC(C(=O)O)N1CCN(CC(=O)O)CCN(CC(=O)O)CCN(CC(=O)O)CC1] |
| DTPA | 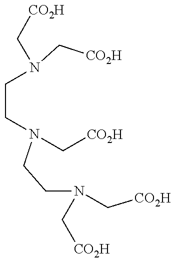 [SMILES: O=C(O)CN(CCN(CC(=O)O)CC(=O)O)CCN(CC(=O)O)CC(=O)O] |
| CHX-A″-DTPA | 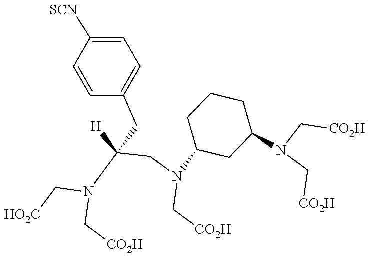 |
| ca-DTPA | 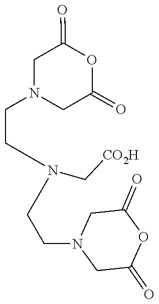 [SMILES: O=C(O)CN(CCN1CC(=O)OC(=O)C1)CCN1CC(=O)OC(=O)C1] |
| ibca-DTPA | 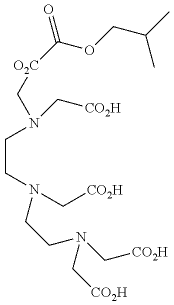 |
| 1B4M-DTPA | 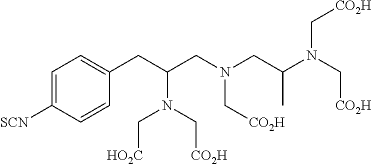 |
| lys-DTPA | 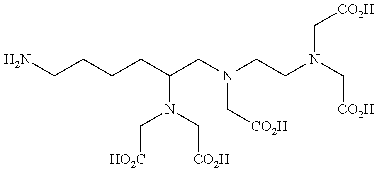 [SMILES: NCCCCC(CN(CCN(CC(=O)O)CC(=O)O)CC(=O)O)N(CC(=O)O)CC(=O)O] |
| vinyl DTPA | 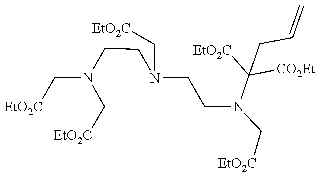 [SMILES: C=CCC(C(=O)OCC)(C(=O)OCC)N(CCN(CCN(CC(=O)OCC)CC(=O)OCC)CC(=O)OCC)CC(=O)OCC] |
| glu-DTPA | 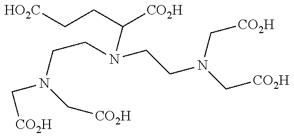 [SMILES: O=C(O)CCC(C(=O)O)N(CCN(CC(=O)O)CC(=O)O)CCN(CC(=O)O)CC(=O)O] |
| NOTA | 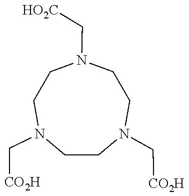 [SMILES: O=C(O)CN1CCN(CC(=O)O)CCN(CC(=O)O)CC1] |
| p-NCS-Bz- NOTA | 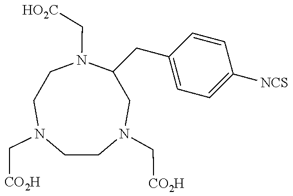 |
| N-NOTA | 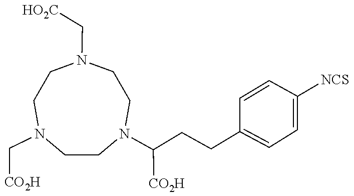 |
| NODAGA | 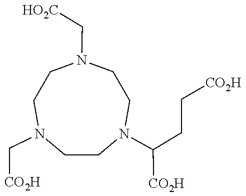 [SMILES: O=C(O)CCC(C(=O)O)N1CCN(CC(=O)O)CCN(CC(=O)O)CC1] |
| NODASA | 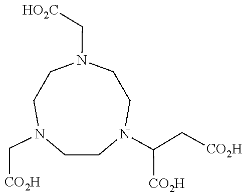 [SMILES: O=C(O)CC(C(=O)O)N1CCN(CC(=O)O)CCN(CC(=O)O)CC1] |
| triaza- cyclononane- TM | 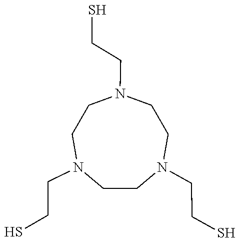 [SMILES: SCCN1CCN(CCS)CCN(CCS)CC1] |
| NOTP | 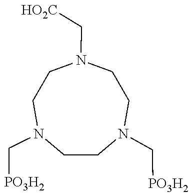 |
| HYNIC | 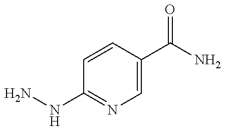 [SMILES: NNc1ccc(C(N)=O)cn1] |
| EDTA | 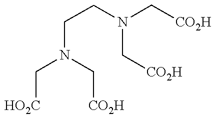 [SMILES: O=C(O)CN(CCN(CC(=O)O)CC(=O)O)CC(=O)O] |
| Deferoxamine (DFO) | 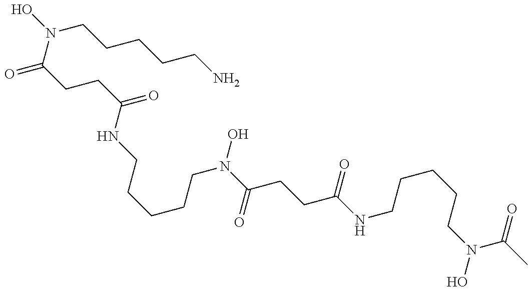 [SMILES: CC(=O)N(O)CCCCCNC(=O)CCC(=O)N(O)CCCCCNC(=O)CCC(=O)N(O)CCCCCN] |
| DFO-pPhe- NCS | 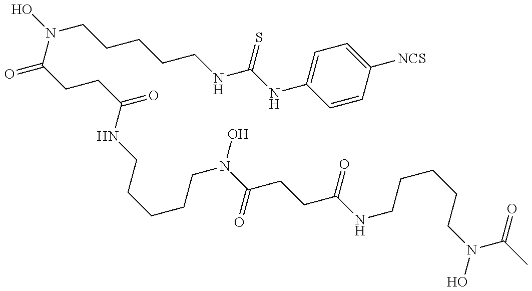 |
| DFOcyclostar | 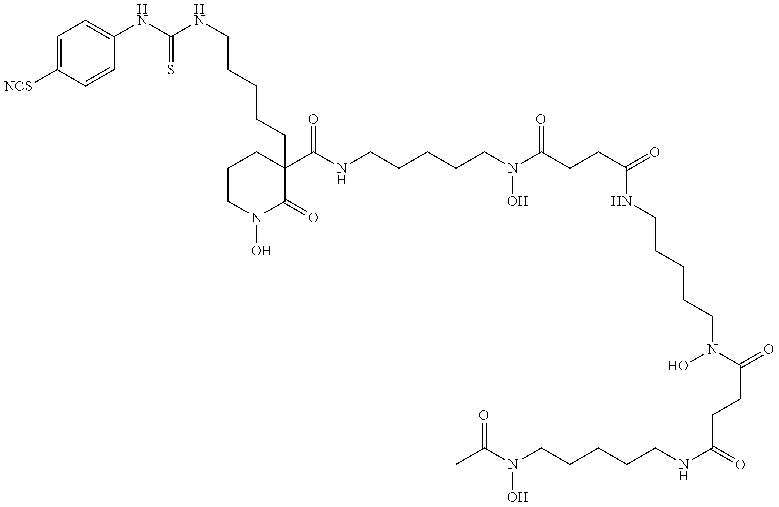 |
| DFO* | 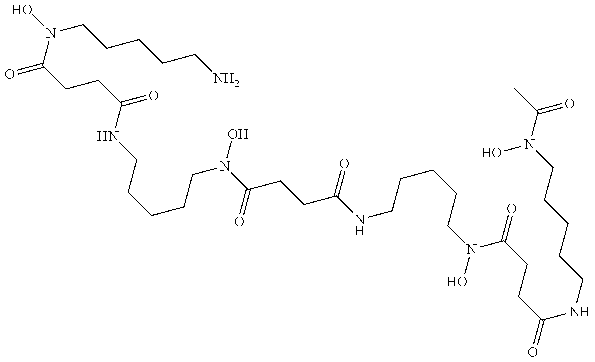 [SMILES: CC(=O)N(O)CCCCCNC(=O)CCC(=O)N(O)CCCCCNC(=O)CCC(=O)N(O)CCCCCNC(=O)CCC(=O)N(O)CCCCCN] |
| TAME | 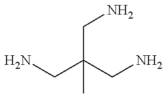 |
| TAME Hex | 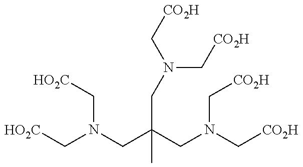 [SMILES: CC(CN(CC(=O)O)CC(=O)O)(CN(CC(=O)O)CC(=O)O)CN(CC(=O)O)CC(=O)O] |
| O- hydroxybenzyl iminodiacetic acid | 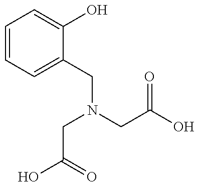 [SMILES: O=C(O)CN(CC(=O)O)Cc1ccccc1O] |
| TACN | 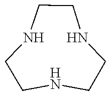 |
| Cyclen | 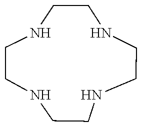 [SMILES: C1CNCCNCCNCCN1] |
| TETA | 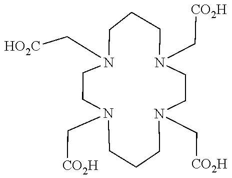 [SMILES: O=C(O)CN1CCCN(CC(=O)O)CCN(CC(=O)O)CCCN(CC(=O)O)CC1] |
| 2C-TETA | |
| 6C-TETA | |
| PEPA | |
| BF-PEPA | 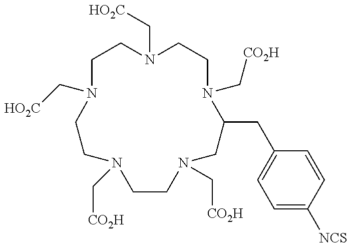 |
| HEHA | 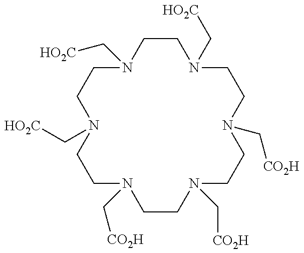 [SMILES: O=C(O)CN1CCN(CC(=O)O)CCN(CC(=O)O)CCN(CC(=O)O)CCN(CC(=O)O)CCN(CC(=O)O)CC1] |
| BF-HEHA | 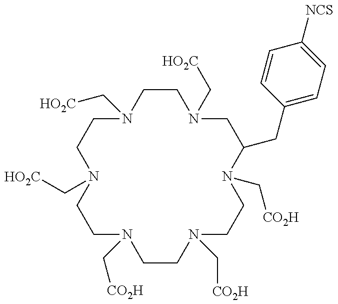 |
| TCMC | 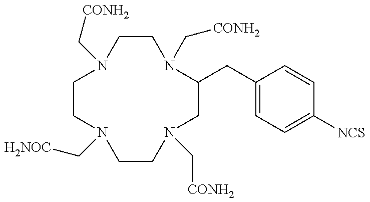 |
| Macropa | 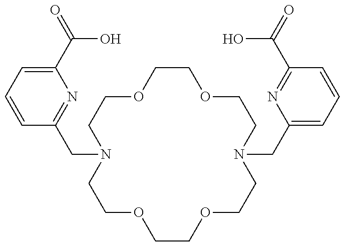 [SMILES: O=C(O)c1cccc(CN2CCOCCOCCN(Cc3cccc(C(=O)O)n3)CCOCCOCC2)n1] |
| Macropa-NCS | 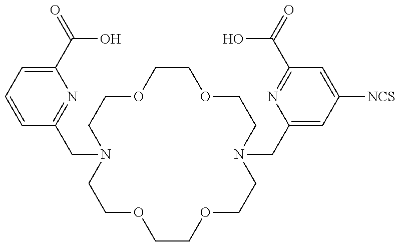 |
| Macrodipa | 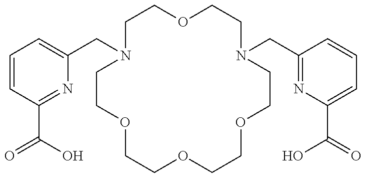 [SMILES: O=C(O)c1cccc(CN2CCOCCOCCOCCN(Cc3cccc(C(=O)O)n3)CCOCC2)n1] |
| Crown | 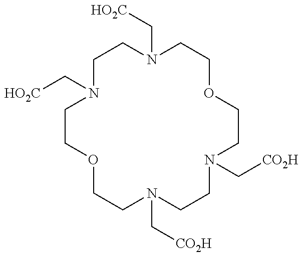 [SMILES: O=C(O)CN1CCOCCN(CC(=O)O)CCN(CC(=O)O)CCOCCN(CC(=O)O)CC1] |
| | 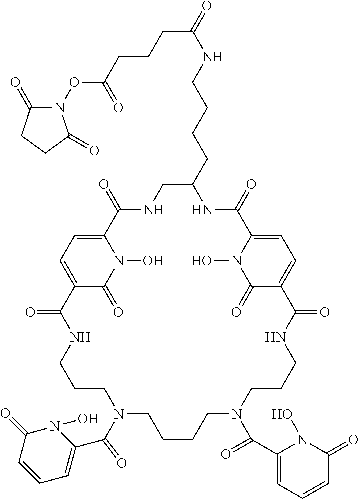 [SMILES: O=C(CCCC(=O)ON1C(=O)CCC1=O)NCCCCC1CNC(=O)c2ccc(c(=O)n2O)C(=O)NCCCN(C(=O)c2cccc(=O)n2O)CCCCN(C(=O)c2cccc(=O)n2O)CCCNC(=O)c2ccc(n(O)c2=O)C(=O)N1] |
| HOPO | 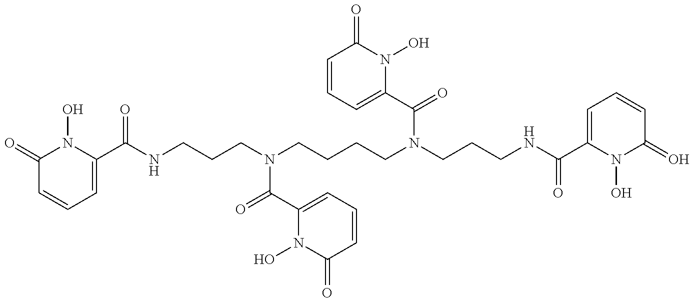 [SMILES: O=C(NCCCN(CCCCN(CCCNC(=O)c1cccc(=O)n1O)C(=O)c1cccc(=O)n1O)C(=O)c1cccc(=O)n1O)c1cccc(=O)n1O] |

In some embodiments, chelating agents include acyclic or macrocyclic compounds. Non-limiting examples of chelating agents include 1,4,7,10-tetraazacyclododecane-1,4,7,10-tetraacetic acid (DOTA); DOTA derivative: DO3A; diethylenetriamine-N,N,N′,N″,N″-pentaacetic acid (DTPA); DTPA derivatives: 2-(p-SCN-Bz)-6-methyl-DTPA, CHX-A″-DTPA, and the cyclic anhydride of DTPA (CA-DTPA); 1,4,7-triazacyclononane-1,4-7-triacetic acid (NOTA); NOTA derivatives (e.g., BCNOTA, p-NCS-Bz-NOTA, BCNOT); 6-hydrazinonicotinamide (HYNIC); ethylenediamine tetraacetic acid (EDTA); N,N′-ethylene-di-L-cysteine; N,N′-bis(2,2-dimethyl-2-mercaptoethyl)ethylenediamine-N,N′-diacetic acid (6SS); 1-(4-carboxymet'oxybenzyl)-N-N′-bis[(2-mercapto-2,2-dimethyl)ethyl]-1,2-ethy'enediamine-N,N′-diacetic acid (B6SS); Deferoxamine (DFO); 1,1,1-tris(aminomethyl)ethane (TAME); tris(aminomethyl)ethane-N,N,N′,N′,N″,N″-hexaacetic acid (TAME Hex); O-hydroxybenzyl iminodiacetic acid; 1,4,7-triazacyclononane (TACN); 1,4,7,10-tretraazacyclododecane (cyclen); 1,4,7-triazacyclononane-1-succinic acid-4,7-diacetic acid (NODASA); 1-(1-carboxy-3-carboxypropyl)-4,7-bis-(carboxymethyl)-1,4,7-triazacyclononane (NODAGA); 1,4,7-tris(2-mercaptoethyl)-1,4,7-triazacylclonane (triazacyclononane-TM); 1,4,7-triazacyclononane-N,N′,N″-tris(methylenephosphonic)acid (NOTP); 1,4,8,11-tetraazacyclotetradecane-N,N″,N″,N″-tetraacetic acid (TETA); 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA); 1,4,7,10-tetrakis(carbamoylmethyl)-1,4,7,10-tetraazacyclododecane (TCMC); and derivatives or analogs thereof.

In an embodiment, Chelator is independently selected from a group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N,N′,N″,N′″,N″″,N′″″-hexaacetic acid (HEHA). In another embodiment, Chelator is selected from Deferoxamine (DFO), 5,11,16,22-Tetraazahexacosanediamide (DFO*), and N,N′-1,4-Butanediylbis[N-[3-[[(1,6-dihydro-1-hydroxy-6-oxo-2-pyridinyl)carbonyl]amino]propyl]-1,6-dihydro-1-hydroxy-6-oxo-2-pyridinecarboxamide] (HOPO).

In some embodiments, chelating agents of the present disclosure include DOTA, DOTAGA, or any derivative/analog thereof. Any chelating agent disclosed in Eisenwiener et al., Bioorg. Med. Chem. Lett., vol. 10(18):2133 (2000), the contents of which are incorporated herein by reference in their entirety, may be used as a chelating agent.

Chelators such as DOTA can be attached to any place of the cyclic peptide without negatively affecting the binding of the cyclic peptide to its targets (i.e., one of skill in the art would be able to discern how placement of the chelator affects the binding by performing the studies described herein). In some embodiments, chelators such as DOTA can be attached directly to the N-terminal amine or to a short linker attached to that same residue. Alternatively, chelators such as DOTA can be attached via a short linker to the C-terminus or to a side chain that can tolerate its presence. In some embodiments, a crosslinker, such as dibromoxilene, that has previously prefunctionlaized with a chelator moiety can be attached to the cyclic peptide.

### Linkers

Targeting construct may include optional linkers connecting chelating agents and targeting moieties. Targeting construct linkers may link one or more chelating agents and one or more targeting moieties. Linkers may include one or more of an ester, disulfide, amide, acylhydrazone, ether, carbamate, carbonate, sulfonamide, alkyl, aryl, heteroaryl, thioether, and urea.

In some embodiments, linkers include cleavable linkers. In some embodiments, linkers include non-cleavable linkers. In some embodiments, optional linkers include amino acids.

As used herein, the term “linker” refers to a chemical moiety that joins a chelator to a peptide of the present disclosure. Any suitable linker known to those skilled in the art in view of the present disclosure can be used herein.

Linkers can act as electrophiles and bond to a nucleophilic portion of a chelator. Alternatively, linkers can act as nucleophiles and bond to an electrophilic portion of a chelator. It is understood that a linker may be attached to a chelator via the carbon backbone of the chelator allowing all “binding arms” of the chelator molecule to interact with the metal. Alternatively, one of the arms may be attached to the linker.

For example, if a chelator is bound, via an amine group of the cyclic peptide or optional linker, to a carbonyl of the chelator, then an amide bond is formed between the chelator and the cyclic peptide or optional linker.

In another example, when a chelator is DOTA and linker is PEG, then the resulting structure can be:

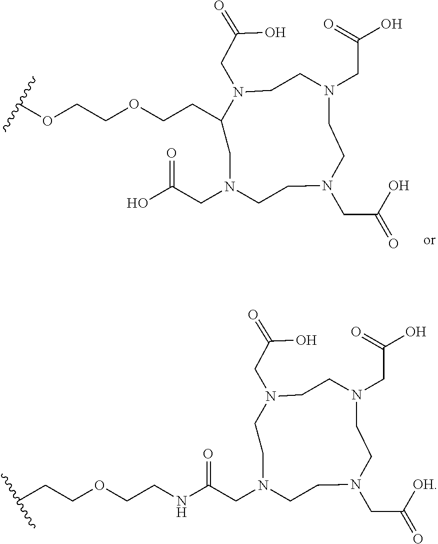 [SMILES: *OCCOCCC1CN(CC(=O)O)CCN(CC(=O)O)CCN(CC(=O)O)CCN1CC(=O)O ; *CCOCCNC(=O)CN1CCN(CC(=O)O)CCN(CC(=O)O)CCN(CC(=O)O)CC1]

In yet another example, when a chelator is DOTA and the amino acid is Lys, then the resulting sidechain of the amino acid can be:

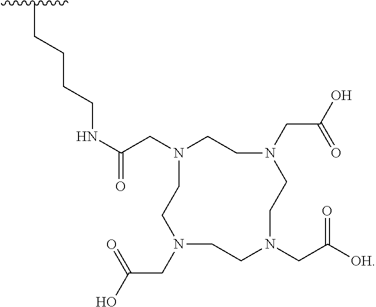 [SMILES: *CCCCNC(=O)CN1CCN(CC(=O)O)CCN(CC(=O)O)CCN(CC(=O)O)CC1]

### Cargos

Targeting constructs may include a variety of cargo. In some embodiments, cargo association with targeting constructs is facilitated by chelating agents. Cargo may include radioactive agents. Radioactive agent cargo associated with targeting constructs via chelating agents may include radionuclides and/or radioisotopes. Chelating agents used for targeting construct association with such cargo may include metal chelating groups. In an embodiment, the chelator of the compounds provided herein further comprises a radiometal ion bound to the chelator via coordinate bonding, thereby forming a radiometal complex.

A variety of radionuclides have emission properties, including α, β, γ, and Auger emissions. These may be used with targeting constructs used for therapeutic and/or diagnostic purposes. For example, active agent Z may include Y-90, Y-86, 1-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and/or Pt-199 radioisotopes.

In some embodiments, targeting constructs used in imaging applications may include radioisotope cargo useful as imaging probes. Non-limiting examples of such radioisotopes include, but are not limited to, 1-124, 1-131, In-111, Re-186, Re-188, Y-90, Bi-212, At-211, Sr-89, Ho-166, Sm-153, Cu-60, Cu-67, Cu-64, Lu-177, Ac-225, Bi-213, Th-227, Pb-212, Ra-223, P-32, Sc-47, Br-76, Br-77, Rh-105, Pd-103, Ag-111, Pr-142, Pm-149, Gd-159, In-111, Ir-194, Pt-199, Tc-99m, Co-57, Ga-66, Ga-67, Ga-68, Kr-81m, Rb-82, Sr-92, TI-201,Y-86, Zr-89, C-11, N-13, 0-15 and F-18.

In some embodiments, targeting construct cargo includes any of the radioisotopes listed in Table 3 including radionuclide parents and daughters thereof.

TABLE 3
Radioisotopes
Radioisotopes

| | |
|---|---|
| C-11 | Rh-105 |
| N-13 | Ag-111 |
| O-15 | In-111 |
| F-18 | I-124 |
| P-32 | I-131 |
| Sc-47 | Pr-142 |
| Co-57 | Pm-149 |
| Cu-60 | Sm-153 |
| Cu-67 | Gd-159 |
| Cu-64 | Ho-166 |
| Ga-66 | Lu-177 |
| Ga-67 | Re-186 |
| Ga-68 | Re-188 |
| Br-76 | Ir-194 |
| Br-77 | Pt-199 |
| Kr-81m | Tl-201 |
| Rb-82 | Pb-203 |
| Y-86 | At-211 |
| Zr-89 | Pb-212 |
| Sr-89 | Bi-212 |
| Y-86 | Bi-213 |
| Y-90 | Ra-223 |
| Sr-92 | Ac-225 |
| Tc-99m | Th-227 |
| Pd-103 | Lu-175 |
| Ac-227 | In-115 |

In some embodiments, the radioisotope is referred to as a radionuclide. In some embodiments, the radionuclide is a therapeutically active radionuclide. Suitable therapeutically active radionuclides include, but are not limited to, ³²P, ⁶⁷Cu, ¹⁸⁶Re, ¹⁸⁸Re, ⁸⁹Sr, ⁹⁰Y, ¹⁴³Ce, ¹⁷⁷Lu, ¹⁶¹Tb, ¹⁶⁶Ho, ¹⁶⁹Er, ¹⁸³Ta, ¹⁵³Sm, ²¹³Bi, ¹³¹I, ¹⁴⁹Tb, ⁴⁷Sc, ²²⁵Ac, ²¹²Pb, ²¹¹At, ²²³Ra, ²²⁷Th, and ²²⁶Th. In some embodiments, the radionuclide is a therapeutically active radionuclide selected from ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, and ²²⁷Th. In particular embodiments, the radionuclide is a therapeutically active radionuclide selected from ⁹⁰Y, ¹⁷⁷Lu, ¹³¹I, ²²⁵Ac, ²¹¹At, and ²²⁷Th. In a particular embodiment, the therapeutically active radionuclide is ¹⁷⁷Lu.

Alternatively, in some embodiments, the radionuclide is a diagnostically active radionuclide. Suitable diagnostically active radionuclides include, but are not limited to, ¹¹¹In, ^{99m}Tc, ^{94m}Tc, ⁶⁷Ga, ⁶⁸Ga, ²⁰³Pb, ⁶⁴Cu, ⁸⁶Y, ⁸⁹Zr, ⁵¹Mn, ⁵²Mn, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, and ²⁰¹Tl. In some embodiments, the radionuclide is a diagnostically active radionuclide selected from ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, and ⁴³Sc.

In particular embodiments, the radionuclide is a diagnostically active radionuclide selected from ¹¹¹In, ^{99m}Tc, ⁶⁸Ga, ⁶⁴Cu, ⁸⁹Zr, ¹²³I, ¹²⁴I, and ¹⁸F. In a particular embodiment, the diagnostically active radionuclide is ⁶⁸Ga. In another particular embodiment, the diagnostically active radionuclide is ¹⁸F.

In another particular embodiment, the diagnostically active radionuclide is ⁶⁴Cu.In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac.

In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th.

In some embodiments, the radionuclide is ⁶⁶Ga, ⁶⁷Ga, ⁶⁸Ga, ⁶⁴Cu, ¹⁷⁷Lu, or ²²⁵Ac. In some embodiments, the radionuclide is ¹¹¹In, ^{99m}Tc, ⁶⁸Ga, ⁶⁴Cu, ⁸⁹Zr, ¹²³I, ¹²⁴I, ¹⁸F, ⁹⁰Y, ¹⁷⁷Lu, ¹³¹I, ²²⁵Ac, ²¹¹At, or ²²⁷Th.

In certain embodiments, the radionuclide is ¹⁷⁷Lu. In certain embodiments, the radionuclide is ²²⁵Ac. In certain embodiments, the radionuclide is ⁶⁸Ga. In certain embodiments, the radionuclide is ¹⁸F.

In some embodiments, the radionuclide is ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th.

In some embodiments, the radionuclide is a radiohalogen, e.g., ¹⁸F, ⁷⁵Br, ⁷⁶Br, ⁷⁷Br, ⁸⁰Br, ^{80m}Br, ⁸²Br, ¹²³I, ¹²⁴I, ¹²⁵I, ¹³¹I, or ²¹¹At. When the radionuclide is a radiohalogen, the term radiohalogen includes complexes that make the radiohalogen suitable for covalent attachment to the linker or the cyclic peptide or for chelation or complex formation with the chelator. Such complexes contemplated under the term radiohalogen include Si—¹⁸F, B—¹⁸F, and Al—¹⁸F.

In some embodiments, the radiohalogen is connected directly to the cyclic peptide or the linker. For example, ¹³¹I and ¹⁸F (or any other radiohalogen) can be substituted at any position of the linker or the cyclic peptide suitable for substitution with a halo group. In some embodiments, the radiohalogen is ¹⁸F. In some embodiments, when a radiohalogen is connected directly to the cyclic peptide or the linker, the chelator is absent.

In some embodiments, targeting constructs of the present disclosure can be radiolabeled with a radionuclide at any site of peptide that targets DLL3. For example, in some embodiments the peptide that targets DLL3 is conjugated directly to a radionuclide. In one embodiment, the radionuclide is covalently attached to the peptide that targets DLL3. In another embodiment, the radionuclide can rely on ionic interactions, thereby forming a peptide radionuclide salt.

In some embodiments, the peptide that targets DLL3 can be conjugated to a chelator. In one embodiment, the peptide that targets DLL3 can be radiolabeled via chelation of the radionuclide to the chelator. Chelation of a radionuclide to a chelator may be depicted using solid single bonds, dashed single bonds, or a combination thereof. For example, the chelation of a radionuclide to DOTA can be depicted below with solid single bonds or dashed single bonds. In some embodiments, the charge may also be indicated. For example, when a radionuclide is chelated to a chelating agent, each of the groups chelating the radionuclide may have a negative charge and the radionuclide being chelated may have an opposing positive charge. Such bonds and charges may be depicted herein as follows in the case of ⁶⁸Ga:

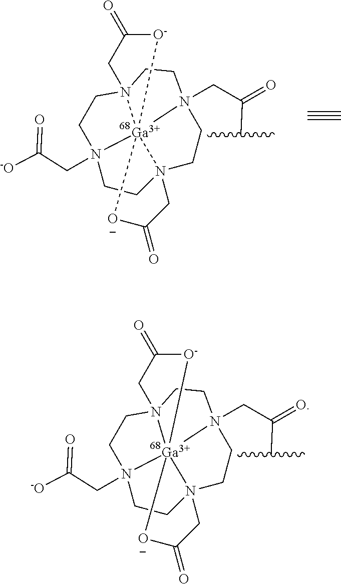

In the case of ²²⁵Ac, such bonds and charges may be illustrated (non-limiting) as follows:

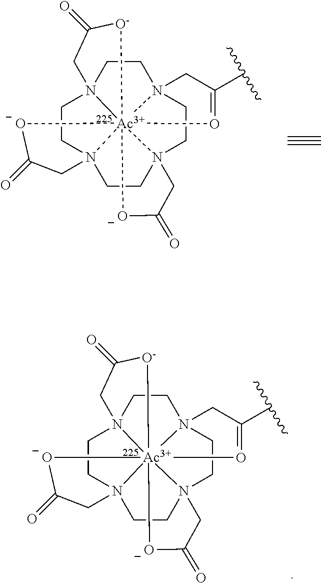

The radionuclide may be a therapeutic radionuclide, diagnostic radionuclide, or both. Suitable radionuclides include, but are not limited to, auger-electron emitting radionuclides, β-emitting (beta-plus or beta-minus-emitting) radionuclides, and α-emitting (alpha-emitting) radionuclides. The selection of the type of radionuclide may depend on the use of the peptide that targets DLL3. As will be appreciated by the skilled artisan, several factors may be considered when selecting a radionuclide for use in a peptide that targets DLL3, such as, for example, the half-life, the linear energy transfer, the imaging capabilities, and the emission range in tissue. For example, β-emitting radionuclides typically have a longer emission range in tissue (e.g., 0.1-10 mm) and emit photons in an energy range that is easily imaged, and as such, they may be selected for use in a DLL3-targeting compound being used for therapeutic, diagnostic, or theragnostic purposes. On the other hand, α-emitting radionuclides have a shorter emission range in tissue (e.g., 50-100 micrometer) and a high potency due to the amount of energy deposited per path length traveled (i.e., linear energy transfer), which is approximately 400 times greater than that of electrons (beta-minus particles) or positrons (beta-plus particles). Thus, α-emitting radionuclides may be selected for therapeutic uses in which high potency of the radionuclide is desired.

Accordingly, in some embodiments, the radionuclide is an α-emitting radionuclide. In other embodiments, the radionuclide is a β-emitting radionuclide. In yet other embodiments, the radionuclide is an auger-electron emitting radionuclide.

### Targeting Constructs

In some embodiments, the present disclosure provides constructs capable of localizing to and/or associating with targets. Such constructs that include any combination of a targeting moiety and a cargo are referred to herein as “targeting constructs.” Provided herein the targeting constructs can be directed to DLL3.

As used herein, the term “targeting moiety” refers to a component of a targeting construct or combination of components involved in targeting construct localization to or association with a target. Cargo components of targeting constructs may include any one of a variety of compounds, including, but not limited to, chemical compounds, biomolecules, metals, polymeric molecules, therapeutic agents, cytotoxic agents, and radioactive agents. In a particular embodiment, the targeting construct comprises a targeting moiety that is a cyclic peptide that targets DLL3, which is attached, via an optional linker, to a chelating agent for association of a radioisotope.

Targeting constructs of the present disclosure may include chelating agents. As used herein, the term “chelating agent” refers to any compound capable of forming two or more bonds with metal atoms. Chelating agents may facilitate targeting construct association with cargo that includes metal atoms. In a particular embodiment, the targeting construct comprises a chelating agent for association of a radioisotope.

The terms “chelated to” and “complexed with” as used herein is meant to indicate that two independent constituents are joined together such as by one or more non-covalent bonds, e.g., coordination bonds.

The term “radiolabeled” or “labeled” as used herein means that a non-radioactive compound is labeled with a radioisotope. Radiolabeling can be achieved, e.g., via chelation or complexation of a chelator with an appropriate radionuclide. Radiolabeling can also refer to chemically substituting one group on a compound for a radionuclide, e.g., by forming a covalent bond, such as, e.g., in the case of ¹⁸F.

Targeting construct components may be associated via one or more linkers. For example, targeting moieties may be associated with chelating agents or cargo via linkers. In some embodiments, linkers include chelating agents (e.g., where targeting construct cargo includes metal atoms).

In some embodiments, targeting constructs of the present disclosure include targeting moieties attached, optionally by a linker, to a cargo or a chelating agent for association of cargo. Targeting constructs may include a single targeting moiety and a single chelating agent, i.e., having the structure TM-L-CA, where “TM” is a targeting moiety, “L” is an optional linker, and “CA” is a chelating agent. Alternatively, targeting constructs may include a single targeting moiety and more than one chelating agent, e.g., a construct having the structure TM-L-(CA)n, wherein n is an integer representing the number of chelating agents. In some embodiments, n is an integer between 1 and 50, such as between 2 and 20, or between 1 and 5. Targeting constructs may have a structure of CA-L-TM-L-CA, wherein each L and each CA may be the same or different.

Targeting constructs optionally associated with radioactive cargo may be referred to herein according to corresponding analogs, i.e., the “radioactive analog” or the “non-radioactive analog” of a given targeting construct.

In some embodiments, targeting constructs may include detectable labels. Detectable labels may be used to detect antibody binding. Examples of detectable labels include, but are not limited to, radioisotopes, fluorophores, chromophores, chemiluminescent compounds, enzymes, enzyme co-factors, dyes, metal ions, ligands, biotin, avidin, streptavidin, haptens, quantum dots, or any other detectable labels known in the art or described herein.

### Formulations

In some embodiments, compositions are administered to humans, human patients, or subjects. For the purposes of the present disclosure, the phrase “active ingredient” generally refers to the constructs as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping, and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The constructs of the present disclosure can be formulated using one or more excipients to: (1) increase stability; (2) permit the sustained or delayed release; (3) alter the biodistribution; (4) alter the release profile of the compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the disclosure may include one or more excipients, each in an amount that together increases the stability of the compounds.

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc., and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, and mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Administration

The constructs of the present disclosure may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

The formulations described herein contain an effective amount of constructs in a pharmaceutical carrier appropriate for administration to an individual in need thereof. The formulations may be administered parenterally (e.g., by injection or infusion). The formulations or variations thereof may be administered in any manner including enterally, topically (e.g., to the eye), or via pulmonary administration. In some embodiments the formulations are administered topically.

### Dosing

The present disclosure provides methods comprising administering constructs as described herein to a subject in need thereof. Constructs as described herein may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

The concentration of the constructs may be between about 0.01 mg/mL to about 50 mg/mL, about 0.1 mg/mL to about 25 mg/mL, about 0.5 mg/mL to about 10 mg/mL, or about 1 mg/mL to about 5 mg/mL in the pharmaceutical composition.

As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In some embodiments, the total dose (over the course of a treatment regimen) of the DLL3 targeting construct comprising a β-emitter such as, e.g., ¹⁷⁷Lu, is from about 1 GBq to about 200 GBq. In some embodiments, the DLL3 targeting construct comprising a β-emitter is administered in a total dose to deliver from 40 to 100 GBq of radiation. In some embodiments, the DLL3 targeting construct comprising the β-emitter is administered in a single dose (once within a 24-hour period) to deliver from about 1 to about 20 GBq of radiation. In some embodiments, DLL3 targeting construct comprising the β-emitter is administered in a single dose (once within a 24-hour period) to deliver from about 3 to about 15 GBq of radiation. In some embodiments, DLL3 targeting construct comprising the β-emitter is administered in a single dose (once within a 24-hour period) to deliver from about 5 to about 10 GBq of radiation.

In some embodiments, the total dose (over the course of a treatment regimen) of the DLL3 targeting construct comprising an α-emitter, e.g., ²²⁵Ac, is from about 1 MBq to about 100 MBq, e.g., about 4 MBq to about 80 MBq, e.g., about 5 MBq to about 77 MBq, e.g., about 5 MBq, about 6 MBq, about 8 MBq, about 10 MBq, about 13 MBq, and about 76 MBq. In some embodiments, the DLL3 targeting construct comprising an α-emitter is administered in a total dose of from about 20 to about 80 MBq of radiation. In some embodiments, the DLL3 targeting construct comprising an α-emitter is administered in a single dose (once within a 24-hour period) to deliver from about 1 to about 40 MBq of radiation. In some embodiments, the DLL3 targeting construct comprising an α-emitter is administered in a single dose (once within a 24-hour period) to deliver from about 5 to about 40 MBq of radiation. In some embodiments, DLL3 targeting construct comprising an α-emitter is administered in a single dose (once within a 24-hour period) to deliver from about 5 to about 25 MBq of radiation.

In some embodiments, the total dose (over the course of a treatment regimen) of the DLL3 targeting construct comprising an α-emitter, e.g., ²²⁵Ac, is administered to the subject once about every 4 to 10 weeks. In another embodiment, the construct is administered to the subject once about every 6 to 8 weeks. In still another embodiment, the construct is administered to the subject once about every 6 weeks. In yet another embodiment, the construct is administered to the subject once about every 6 weeks for 4 to 6 cycles.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous)

### II. Methods

In some embodiments, the present disclosure provides methods related to preparing, using, and evaluating compounds (e.g., targeting constructs) and compositions disclosed herein.

### Therapeutic Applications

In some embodiments, methods of the present disclosure include methods of treating therapeutic indications using compounds and/or compositions disclosed herein. As used herein, the term “therapeutic indication” refers to any symptom, condition, disorder, or disease that may be alleviated, stabilized, improved, cured, or otherwise addressed by some form of treatment or other therapeutic intervention. In some embodiments, methods of the present disclosure include treating therapeutic indications by targeting constructs disclosed herein.

By “lower” or “reduce” in the context of a disease marker or symptom is meant a significant decrease in such a level, often statistically significant. The decrease may be, for example, at least 10%, at least 20%, at least 30%, at least 40% or more, and is preferably down to a level accepted as within the range of normal for an individual without such a disorder.

By “increase” or “raise” in the context of a disease marker or symptom is meant a significant rise in such level, often statistically significant. The increase may be, for example, at least 10%, at least 20%, at least 30%, at least 40% or more, and is preferably up to a level accepted as within the range of normal for an individual without such disorder.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a compound or composition described herein, “effective against” a disease or disorder indicates that administration in a clinically appropriate manner results in a beneficial effect for at least a fraction of patients, such as an improvement of symptoms, a cure, a reduction in disease load, extension of life, improvement in quality of life, or other effect generally recognized as positive by medical doctors familiar with treating the particular type of disease or disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given compound or composition may also be judged using an experimental animal model for the given disease as known in the art. When using an experimental animal model, efficacy of treatment is evidenced when a statistically significant modulation in a marker or symptom is observed.

In some embodiments, methods of the present disclosure include administering targeting constructs described herein to treat hyperproliferative diseases, metabolic diseases, infectious diseases, and/or cancer. Targeting construct formulations may be administered by multiple routes, including, but not limited to, injection, oral administration, or topical administration. In some embodiments, administration is to a mucosal surface (lung, nasal, oral, buccal, sublingual, vaginally, rectally) or to the eye (intraocularly or transocularly).

### Cancer

In an aspect, provided herein is a method of targeting DLL3 in a subject in need thereof, comprising administering to the individual a therapeutically effective amount of a compound disclosed herein.

In another aspect, provided herein is a method of treating cancer in a subject in need thereof, comprising administering to the individual a therapeutically effective amount of a compound disclosed herein.

In an embodiment, the cancer is a DLL3-mediated cancer. In another embodiment, the cancer is a DLL3-expressing cancer. In another embodiment, the cancer is small cell lung cancer, urothelial cancer, melanoma, or squamous cell carcinoma.

In another embodiment neuroendocrine neoplasm is selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma (MCC), breast cancer, Non-Hodgkin lymphoma, Hodgkin lymphoma, Head & Neck tumor, urothelial carcinoma (bladder), Renal Cell Carcinoma, Hepatocellular Carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, esthesioneuroblastoma, functional carcinoid tumor, insulinoma, gastrinoma, vasoactive intestinal peptide (VIP) oma, glucagonoma, serotoninoma, histaminoma, ACTHoma, pheocromocytoma, and somatostatinoma. In another embodiment, the cancer is a neuroendocrine neoplasm, melanoma, or primary brain cancer. In another embodiment, the neuroendocrine neoplasm is selected from small cell lung cancer (SCLC, including, e.g., Extensive-stage (ES)-SCLC or Limited-stage (LS)-SCLC), medullary thyroid carcinoma (MTC), large cell neuroendocrine cancer (LCNEC), gastroenteropancreatic neuroendocrine carcinoma (GEP NEC), neuroendocrine prostate cancer (NEPC), e.g., treatment emergent NEPC, small cell prostate cancer (SCPC), Merkel cell carcinoma (MCC), neuroendocrine cervical carcinoma, and Grade 3 neuroendocrine tumors (NETs). In some embodiments, the extrapulmonary neuroendocrine carcinoma (NEC) of the cervix. In some embodiment, the small cell lung cancer can be extensive-stage (ES)-SCLC or Limited-stage (LS)-SCLC. In some embodiments, the neuroendocrine prostate cancer (NEPC), can be treatment emergent NEPC. In some embodiments, the cancer is a solid tumors having DLL3 positivity as measured by immunohistochemistry (IHC) (e.g., ≥1% DLL3 positive cells).

In yet another embodiment, the cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In another embodiment, the cancer is selected from the group consisting of primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningeal cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharyngeal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancy.

In an embodiment, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In another embodiment, the cancer is selected from the group consisting of myeloma, lymphoma, or a cancer selected from gastric, renal, head and neck, oropharyngeal, non-small cell lung cancer (NSCLC), endometrial, hepatocarcinoma, non-Hodgkin's lymphoma, and pulmonary.

In an embodiment, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In an embodiment, the cancer is selected from the group consisting of tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In another aspect, the disclosure provides a compound disclosed herein, or a pharmaceutically acceptable salt thereof, for use in the manufacture of a medicament for treating a disease in which DLL3 plays a role.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer.

In another aspect, provided herein is the use of one or more compounds of the disclosure in the manufacture of a medicament for the treatment of cancer, including without limitation the various types of cancer disclosed herein.

In some embodiments, therapeutic indications include cancer-related indications. The term “cancer” refers to a collection of diseases characterized by dysfunctional cell growth and division, in some cases spreading between bodily regions. As used herein, the term “cancer-related indication” refers to any disease, disorder, or condition pertaining to cancer, cancer treatment, or pre-cancerous conditions. Cancer-related indications include, but are not limited to, pathological conditions characterized by malignant neoplastic growths, tumors, and/or hematological malignancies. In some embodiments, methods of the present disclosure include treatment of cancer-related indications with targeting constructs of the present disclosure.

In various embodiments, methods for treating cancer are provided, wherein the method comprises administering a therapeutically-effective amount of the constructs, salt forms thereof, as described herein, to a subject having a cancer, suspected of having cancer, or having a predisposition to a cancer. According to the present disclosure, cancer embraces any disease or malady characterized by uncontrolled cell proliferation, e.g., hyperproliferation. Cancers may be characterized by tumors, e.g., solid tumors or any neoplasm.

In some embodiments, the subject may be otherwise free of indications for treatment with the constructs. In some embodiments, methods include use of cancer cells, including but not limited to mammalian cancer cells. In some instances, the mammalian cancer cells are human cancer cells.

In some embodiments, constructs according to the present disclosure inhibit cancer and/or tumor growth. They may also reduce one or more of cell proliferation, invasiveness, and metastasis, thereby making them useful for cancer treatment.

In some embodiments, the constructs of the present teachings may be used to prevent the growth of a tumor or cancer, and/or to prevent the metastasis of a tumor or cancer. In some embodiments, compositions of the present teachings may be used to shrink or destroy a cancer.

In some embodiments, the constructs provided herein are useful for inhibiting proliferation of a cancer cell. In some embodiments, the constructs provided herein are useful for inhibiting cellular proliferation, e.g., inhibiting the rate of cellular proliferation, preventing cellular proliferation, and/or inducing cell death. In general, the constructs as described herein can inhibit cellular proliferation of a cancer cell or both inhibiting proliferation and/or inducing cell death of a cancer cell. In some embodiments, cell proliferation is reduced by at least about 25%, about 50%, about 75%, or about 90% after treatment with constructs of the present disclosure compared with cells with no treatment. In some embodiments, cell cycle arrest marker phospho histone H3 (PH3 or PHH3) is increased by at least about 50%, about 75%, about 100%, about 200%, about 400% or about 600% after treatment with constructs of the present disclosure compared with cells with no treatment. In some embodiments, cell apoptosis marker cleaved caspase-3 (CC3) is increased by at least 50%, about 75%, about 100%, about 200%, about 400% or about 600% after treatment with constructs of the present disclosure compared with cells with no treatment.

Furthermore, in some embodiments, constructs of the present disclosure are effective for inhibiting tumor growth, whether measured as a net value of size (weight, surface area or volume) or as a rate over time, in multiple types of tumors.

In some embodiments the size of a tumor is reduced by about 60% or more after treatment with constructs of the present disclosure. In some embodiments, the size of a tumor is reduced by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, by a measure of weight, and/or area and/or volume.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningeal cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2−amplified breast cancer, nasopharyngeal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancy.

In some embodiments, targeting constructs of the present disclosure are used to target cancer cells expressing DLL3. In some embodiments, targeting constructs of the present disclosure are used to treat lung cancer, breast cancer, bladder cancer, colon cancer, urothelial cancer, melanoma, or squamous cell carcinoma.

### Theranostics

“Theranostics,” a term derived from a combination of therapeutics and diagnostics, is an emerging field of medicine where specific disease-targeting agents, e.g., radiopharmaceuticals, may be used to simultaneously or sequentially diagnose and treat medical conditions. Theranostics has become an important field of research and development in medical physics, where varying the isotope of the radionuclide present in a given disease-targeting agent, e.g., a radioligand therapy, can change the disease-targeting agent from an imaging probe (by, e.g., using β+ or γ emitting isotopes to facilitate positron emission tomography (PET) or single photon emission computed tomography (CT) imaging, respectively), to a therapy probe (by, e.g., using α or β− particle or Auger electron emitting isotopes to facilitate targeted radiotherapy).

Molecular imaging is a well-known and useful technique for in vivo diagnostics. It may be used in a wide variety of methods including the three-dimensional mapping of molecular processes, such as gene expression, blood flow, physiological changes (pH, etc.), immune responses and cell trafficking. It can be used to detect and diagnose disease, select optimal treatments, and to monitor the effects of treatments to obtain an early readout of efficacy.

A number of distinct technologies can in principle be used for molecular imaging, including PET, single photon emission tomography (SPET), optical magnetic resonance imaging (MRI), CT, and Cerenkov luminescence imaging (CLI). Combinations of these modalities are emerging to provide improved clinical applications, e.g., PET/CT and SPET/CT (“multi-modal imaging”).

Radionuclide imaging with PET and SPET has the advantage of extremely high sensitivity and small amounts of administered contrast agents (e.g., picomolar in vivo), which do not perturb the in vivo molecular processes. Moreover, the targeting principles for radionuclide imaging can be applied also in targeted delivery of radionuclide therapy. Typically, the isotope that is used as a radionuclide in molecular imaging or therapy is incorporated into a molecule to produce a radiotracer that is pharmaceutically acceptable to the subject.

As such, the constructs of the present disclose can be used in radiotherapy as well as medical imaging for diagnostics.

### Combination Therapies

In some embodiments, constructs of the present disclosure are combined with at least one additional active agent. The active agent may be any suitable drug. The active agent may be selected from the group consisting of hormonetherapeutic agents, anti-neoplastic agents, chemotherapeutic agents, immunotherapeutic agents, immunomodulators, radiosensitizers, DNA damage repair inhibitors, PARP (poly ADP ribose polymerase) inhibitors, and combinations thereof. The constructs and the at least one additional active agent may be administered simultaneously, sequentially, or at any order. The constructs and the at least one additional active agent may be administered at different dosages, with different dosing frequencies, or via different routes, whichever is suitable.

In some embodiments, the additional active agents affect the biodistribution (i.e., tissue distribution) of the constructs of the current disclosure. For example, radioactive agents may accumulate in kidneys and may pose a potential radiotoxicity problem to kidneys and surrounding organs. The additional active agent may reduce renal accumulation or retention time. Preferably, kidney update of the constructs is reduced, while tumor uptake of the constructs is not affected. Kidney and surrounding organs are protected without reducing the efficacy of the constructs. In one non-limiting example, constructs of the current disclosure may be administered in combination with at least one amino acid or analog(s) thereof. The amino acid or analog(s) thereof may be positively charged basic amino acids such as lysine (L-lysine or D-lysine) or arginine, or a combination thereof.

The additional active agent may also be selected from any active agent described herein such as a drug for treating cancer. It may also be a cancer symptom relief drug. Non-limiting examples of symptom relief drugs include: octreotide or lanreotide; interferon, cypoheptadine or any other antihistamines. In some embodiments, constructs of the present disclosure do not have drug-drug interference with the additional active agent. The additional active agent may be administered concomitantly with constructs of the present disclosure.

In some embodiments, a non-radioactive analog of the construct the present disclosure may be combined with a radioactive analog of this construct. For example, the non-radioactive construct can be administered prior to the radioactive analog. In another example, a subject may receive a mixture of the non-radioactive construct and its radioactive analog. In yet another example, a subject may receive the non-radioactive construct treatment first, followed by a mixture of the non-radioactive construct and its radioactive analog.

In some embodiments, a construct of the present disclosure comprising one radiolabel may be combined with at least one other construct of the present disclosure comprising one or more different radiolabels. For example, constructs comprising an imaging radiolabel may be combined with constructs comprising a non-imaging radiolabel. In one embodiment, constructs associated with lutetium (Lu) may be combined with constructs associated with gallium (Ga).

The constructs as described herein or formulations containing the constructs as described herein can be used for the selective tissue delivery of a therapeutic, prophylactic, or diagnostic agent to an individual or patient in need thereof. For example, constructs of the present disclosure are used to deliver radioactive agents to selective tissues. These tissues may be tumor tissues. Dosage regimens may be adjusted to provide the optimum desired response (e.g., a therapeutic or prophylactic response). For example, a single bolus may be administered, several divided doses may be administered overtime or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic effect.

### Diagnostic Applications

In some embodiments, the present disclosure provides diagnostic methods involving use of targeting moieties and or the targeting constructs. Such methods may include detecting DLL3 using any of the targeting moieties and or the targeting constructs described herein. Such methods may include contacting subjects or subject samples with targeting moieties and or the targeting constructs described herein. The peptides and/or targeting constructs may bind to DLL3. Targeting moieties and/or the targeting constructs used for detection methods may include a detectable label. Detection methods may include the use of detection reagents to detect bound antibodies or peptides. As used herein, the term “detection reagent” refers to any compound or substance used to visualize or otherwise observe an object (e.g., a bound antibody or detectable label) or event. Detection reagents may include secondary antibodies or other high affinity compounds (e.g., biotin or avidin) that bind to antibodies being detected or associated conjugates. Detection reagents may be or include substrates for detection of enzymatic detectable labels (e.g., associated with a primary or secondary antibody).

Diagnostic applications of the present disclosure may include detecting DLL3 in subject samples that include cells. In some embodiment cell-associated DLL3 may be detected. Cell-associated DLL3 may be detected in subject samples by fluorescence-associated cell sorting (FACS) analysis. In some embodiments, DLL3 may be detected in subject samples by immunohistochemistry. Such methods may include the use of colorimetric-based systems or immunofluorescence-based systems for DLL3 detection.

In some embodiments, the present disclosure provides methods of stratifying subjects based on detection of DLL3 in subjects or subject samples. Such methods may include detecting DLL3 in subjects or subject samples according to any of the methods described herein (e.g., using peptides or targeting constructs comprising peptides and classifying subjects according to level of DLL3 detected. In a particular embodiment, the targeting construct comprises a targeting moiety that is a cyclic peptide that targets DLL3.

In some embodiments, subjects may be classified according to the presence or absence of DLL3 and/or level of DLL3 in subjects or subject samples. Subjects may be further classified according to the presence or absence of specific DLL3 extracellular subdomains and/or levels of specific DLL3 extracellular subdomains in subjects or subject samples. Classifications used in subject stratification may include, but are not limited to, classifications by disease type, disease prognosis or severity, suitability for treatment, and type of treatment most likely to be successful or appropriate.

### III. Kits and Devices

The disclosure provides a variety of kits and devices for conveniently and/or effectively carrying out methods of the present disclosure. Typically kits will comprise sufficient amounts and/or numbers of components to allow a user to perform multiple treatments of a subject(s) and/or to perform multiple experiments.

In one embodiment, the present disclosure provides kits for inhibiting cancer cell growth in vitro or in vivo, comprising a construct of the present disclosure or a combination of constructs of the present disclosure, optionally in combination with any other active agents.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the constructs in the buffer solution over a period of time and/or under a variety of conditions.

The present disclosure provides for devices which may incorporate constructs of the present disclosure. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver constructs of the present disclosure according to single, multi- or split-dosing regiments. The devices may be employed to deliver constructs of the present disclosure across biological tissue, intradermal, subcutaneously, or intramuscularly.

### IV. Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, 1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “alkylene,” employed alone or in combination with other terms, refers to a divalent alkyl linking group. An alkylene group formally corresponds to an alkane with two C—H bonds replaced by points of attachment of the alkylene group to the remainder of the compound. The term “COₙ-m alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

As used herein, the terms “associated with,” “conjugated,” “linked,” “attached,” and “tethered,” when used with respect to two or more entities, means that the entities are physically associated or connected with one another, either directly or via one or more moieties that serve as linking agents, to form a structure that is sufficiently stable so that the entities remain physically associated, e.g., under working conditions, e.g., under physiological conditions. An “association” need not be through covalent chemical bonding and may include other forms of association or bonding sufficiently stable such that the “associated” entities remain physically associated, e.g., ionic or hydrogen bonding or a hybridization-based connectivity.

In some embodiments, the present disclosure provides methods of stratifying subjects based on detection of DLL3 in subjects or subject samples. Such methods may include detecting DLL3 in subjects or subject samples according to any of the methods described herein (e.g., using peptides or targeting constructs comprising peptides and classifying subjects according to level of DLL3 detected. In a particular embodiment, the targeting construct comprises a targeting moiety that is a cyclic peptide that targets DLL3.

As used herein, the term “cancer” refers to a disease characterized by abnormal cell growth and division.

As used herein, the term “cancer cell” refers to a cell that grows and divides in an abnormal and uncontrolled manner.

As used herein, the term “compound,” refers to a distinct chemical entity. Constructs, targeting constructs, targeting moieties, cargo, chelators, or other construct components, together with any fragments or variants of the foregoing, may be referred to independently or collectively as compounds.

Compounds may exist in one or more isomeric or isotopic forms (including, but not limited to stereoisomers, geometric isomers, tautomers, and isotopes). Compounds may be provided or utilized in singular form or as a mixture of two or more forms (including, but not limited to racemic mixtures of stereoisomers). Some compounds may exist in different forms, which may exhibit different properties and/or activities (including, but not limited to biological activities). For example, compounds containing asymmetrically substituted carbon atoms may be isolated in optically active or racemic forms. As used herein, the below structure indicates the presence of a double bond wherein substituents can be configured as an E or Z isomer:

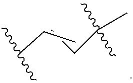

The compounds described herein may be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of compounds of the present disclosure may be isolated as a mixture of isomers or as separated isomeric forms.

Tautomeric compound forms result from the swapping of a single bond with an adjacent double bond and concomitant migration of a proton. Tautomeric forms include prototropic tautomers which are isomeric protonation states having the same empirical formula and total charge. Examples prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein may be provided in forms that include different isotopes of compound atoms. “Isotopes” refers to atoms having the same atomic number but different mass numbers resulting from a different number of neutrons in the nuclei. For example, isotopes of hydrogen include tritium and deuterium.

Compounds described herein may be provided as salts and may be prepared in combination with solvent or water molecules to form solvates and hydrates by routine methods.

As used herein, the term “hydrate” refers to the complex formed by the combining of a compound of Formula I, Formula C, or any Formula disclosed herein, and water.

The term “solvate” refers to a complex formed by the combining of a compound of Formula I, Formula C, or any other Formula as disclosed herein, and a solvent or a crystalline solid containing amounts of a solvent incorporated within the crystal structure. As used herein, the term “solvate” includes hydrates.

As used herein, the term “construct” refers to an artificially manipulated molecule. Some constructs may include nucleic acids and/or peptides, which may be products of recombinant technology and may be artificially synthesized or expressed from a recombinant nucleic acid sequence. Constructs may be combinations of nucleic acids, peptides, and/or other compounds.

As used herein, the term “cyclic” refers to the presence of a continuous loop. Cyclic molecules need not be circular, only joined to form an unbroken chain of subunits. Cyclic peptides may include a “cyclic loop,” formed when two amino acids are connected by a bridging moiety. The cyclic loop comprises the amino acids along the peptide present between the bridged amino acids. Cyclic loops may comprise 2, 3, 4, 5, 6, 7, 8, 9, 10 or more amino acids.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, an “epitope” refers to a surface or region on one or more entities that is capable of interacting with an antibody or other binding biomolecule. For example, a protein epitope may contain one or more amino acids and/or post-translational modifications (e.g., phosphorylated residues) which interact with an antibody. In some embodiments, an epitope may be a “conformational epitope,” which refers to an epitope involving a specific three-dimensional arrangement of the entity(ies) having or forming the epitope. For example, conformational epitopes of proteins may include combinations of amino acids and/or post-translational modifications from folded, non-linear stretches of amino acid chains.

As used herein, the term “equilibrium dissociation constant” or “K_{D}” refers to a value representing the tendency of two or more agents (e.g., two proteins) to reversibly separate. In some cases, K_{D} indicates a concentration of a primary agent at which half of the total levels of a secondary agent are associated with the primary agent.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a peptide or protein; and (4) post-translational modification of a peptide or protein.

As used herein, the term “half-life” or “t_{1/2}” refers to the time it takes for a given process or compound concentration to reach half of a final value. The “terminal half-life” or “terminal t_{1/2}” refers to the time needed for the plasma concentration of a factor to be reduced by half after the concentration of the factor has reached a pseudo-equilibrium.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “identity,” when referring to peptides or nucleic acids, refers to a comparative relationship between sequences. The term is used to describe the degree of sequence relatedness between polymeric sequences, and may include the percentage of matching monomeric components with gap alignments (if any) addressed by a particular mathematical model or computer program (i.e., “algorithms”). Identity of related peptides can be readily calculated by known methods. Such methods include, but are not limited to, those described previously by others (Lesk, A. M., ed., Computational Molecular Biology, Oxford University Press, New York, 1988; Smith, D. W., ed., Biocomputing: Informatics and Genome Projects, Academic Press, New York, 1993; Griffin, A. M. et al., ed., Computer Analysis of Sequence Data, Part 1, Humana Press, New Jersey, 1994; von Heinje, G., Sequence Analysis in Molecular Biology, Academic Press, 1987; Gribskov, M. et al., ed., Sequence Analysis Primer, M. Stockton Press, New York, 1991; and Carillo et al., Applied Math, SIAM J, 1988, 48, 1073).

As used herein, the term “lactam bridge” refers to an amide bond that forms a bridge between chemical groups in a molecule. In some cases, lactam bridges are formed between amino acids in a peptide.

As used herein, a “linker” refers to any chemical structure that connects two or more entities or domains. Linkers may include one or more chemical bonds, atoms, groups of atoms, and/or chemical groups. Examples of chemical groups that can be included in linkers include, but are not limited to, alkyl, alkenyl, alkynyl, amido, amino, ether, thioether, ester, alkylene, heteroalkylene, aryl, or heterocyclyl chemical groups, each of which can be optionally substituted, as described herein. Linkers may include one or more of unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers. Linkers may include amino acids, peptides, peptides, and/or proteins.

Linkers may include carbon chains. Linker carbon chain lengths may be 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more atoms long. Linker carbon chains may contain heteroatoms (e.g., nitrogen, oxygen, sulfur, etc.).

Entities or domains joined by linkers may include, but are not limited to, atoms, chemical groups, nucleosides, nucleotides, nucleobases, sugars, nucleic acids, amino acids, peptides, peptides, proteins, protein complexes, cargo, therapeutic agents, and detectable labels. Linkers may be used for multiple purposes, including, but not limited to, forming multimers or conjugates. For example, compounds contemplated by the present disclosure include those comprising more than one targeting agent and/or more than one cargo. For example, a cyclic peptide disclosed herein may comprise more than one chelator and therefore more than one radionuclide. As another example, a construct disclosed herein may comprise more than one of the targeting cyclic peptides as disclosed herein.

Linkers may include cleavable elements, for example, disulfide (—S—S—) bonds or azo (—N═N—) bonds, which can be cleaved using reducing agents or photolysis. Selectively cleavable bonds may include amido bonds which may be cleaved for example by photolysis or by using tris(2-carboxyethyl)phosphine (TCEP) or other reducing agents. Selectively cleavable bonds may include ester bonds which may be cleaved, for example, by acidic or basic hydrolysis.

Linkers may include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

As used herein, the term “modulation” refers to up regulation (i.e., activation or stimulation) or down regulation (i.e., inhibition or suppression) of a response, or the two in combination or apart. Modulation is generally compared to a baseline or reference that can be internal or external to a treated entity.

As used herein, the term “peptide backbone” consists of repeat units of an amino group, an α-carbon, and a carbonyl group (e.g., —NH₂—CH—C(O—)). As used herein, the term “patient” refers to a subject seeking treatment, in need of treatment, requiring treatment, receiving treatment, expecting treatment, or that are under the care of a trained (e.g., licensed) professional for a particular disease, disorder, or condition. Patients may include any organism. Patient treatments may include, but are not limited to, experimental, diagnostic, prophylactic, and/or therapeutic treatments. Typical patients include, but are not limited to, animals (e.g., mammals such as mice, rats, rabbits, non-human primates, and humans).

As used herein, the term “pharmaceutical composition” refers to a composition comprising at least one active ingredient in a form and amount that permits the active ingredient to be therapeutically effective. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio (e.g., in accordance with the guidelines of government agencies or other regulatory bodies, for example, the U.S. Food and Drug Administration).

The phrase “pharmaceutically acceptable excipient,” as used herein, refers any ingredient other than active agents (e.g., as described herein) present in a pharmaceutical composition and having the properties of being substantially nontoxic and non-inflammatory in a patient.

As used herein, the term “pharmaceutically acceptable salt” refers to derivatives of the disclosed cyclic peptides wherein the parent compound is modified by converting an existing acid or base moiety to its salt form. In some embodiments, the side-chain amino acid groups of the cyclic peptide (e.g., R⁰, R¹, R², R³, R⁴ . . . etc.) can be modified. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “sample” or “biological sample” refers to a subset of its tissues, cells or component parts (e.g., body fluids, including but not limited to blood, serum, plasma, mucus, lymphatic fluid, synovial fluid, cerebrospinal fluid, saliva, amniotic fluid, and urine). Samples may further include a homogenate, lysate, or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, and organs. Samples may further refer to a medium, such as a nutrient broth or gel, which may contain cellular components or other biological materials, such as proteins (e.g., antibodies) or nucleic acid molecules.

As used herein, the term “subject” refers to any entity to which a particular process or activity relates to or is applied. Subjects may include any organism. Typical subjects include, but are not limited to, animals (e.g., mammals such as mice, rats, rabbits, non-human primates, and humans) and/or plants.

As used herein, the term “target” refers to an object or entity to be affected by an action or refers to activity associated with an agent that is directed to the object or entity (e.g., an agent that “targets” an object or entity). In some embodiments, targets refer to antigens, epitopes, or other structures to which antibodies or other compounds bind or that are selected and/or used in the design, development, or isolation of antigen-specific antibodies or other compounds. Targets may include molecular structures that include, but are not limited to, nucleic acids, peptides, proteins, haptens, receptors, carbohydrates, glycans, enzymes, lipids, cells, and fragments or complexes of any of the foregoing.

When used to refer to activity of an agent directed to objects or entities, the term “target” may be used to describe binding activity of agents (e.g., antibodies or related structures) with such objects or entities (e.g., antigens or epitopes). For example, an antibody that binds to a specific antigen may be said to “target” or be “directed to” the particular antigen. Similarly, a compound (e.g., a targeting construct) that exhibits activity (e.g., therapeutic or cytotoxic activity) toward a specific cell or tissue may be said to “target” the cell or tissue.

Targets may include cells (referred to herein as “target cells”). Target cells may be in vivo or in vitro. Target cells may include, for example, blood cells, lymph cells, cells lining the alimentary canal, such as the oral and pharyngeal mucosa, cells forming the villi of the small intestine, cells lining the large intestine, cells lining the respiratory system (nasal passages/lungs) of an animal, dermal/epidermal cells, cells of the vagina and rectum, cells of internal organs, cells of the placenta, and cells of the blood-brain barrier. In some embodiments, target cells may be cancer cells, including, but not limited to those found in leukemias or tumors (e.g., tumors of the brain, lung (small cell and non-small cell), ovary, prostate, breast, and colon, as well as other carcinomas and sarcomas). In still other embodiments, target cells may be part of a tissue. Tissues with target cells or other target structures are referred to herein as target tissues. Target tissues may include, but are not limited to, neuronal tissues, intestinal tissues, pancreatic tissues, liver tissues, kidney tissues, prostate tissues, ovary tissues, lung tissues, bone marrow tissues, and breast tissue tissues.

As used herein, the term “therapeutically effective amount” means an amount of an agent to be delivered that is sufficient, when administered to a subject suffering from or susceptible to a disease, disorder, and/or condition, to treat, improve symptoms of, diagnose, prevent, and/or delay the onset of the disease, disorder, and/or condition.

As used herein the terms “treat,” “treatment,” and the like, refer to any actions taken to offer relief from or alleviation of pathological processes. As it relates to any of the therapeutic indications recited herein, the terms “treat,” “treatment,” and the like mean to relieve or alleviate at least one symptom associated with such indications, or to slow or reverse the progression or anticipated progression of such indications.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “contacting” refers to the bringing together of indicated moieties in an in vitro system or an in vivo system. For example, “contacting” a cell with a compound includes the administration of a compound of the present invention to an individual, subject, or patient, such as a human, as well as, for example, introducing a compound into a sample containing a purified preparation containing the cell.

As used herein, the term “cell” is meant to refer to a cell that is in vitro, ex vivo, or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “tumor” refers to a group of cells forming in solid tissue as a result of abnormal cell growth and division. Benign or “noncancerous” tumors remain isolated while malignant or “cancerous” tumors include cells capable of proliferating to surrounding tissues.

As used herein, the term “tumor cell” refers to a cell associated with or derived from a tumor. Benign or “noncancerous” tumor cells remain associated with tumors while malignant or “cancerous” tumor cells are capable of proliferating to surrounding tissues.

As used herein, the following abbreviations are defined by the structures in Table D.

TABLE D

| Abbreviation | Structure |
|---|---|
| (Cyx)2 | 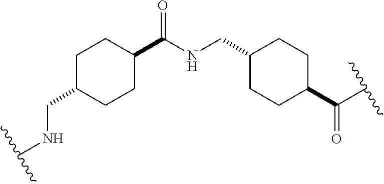 [SMILES: *NC[C@H]1CC[C@H](C(=O)NC[C@H]2CC[C@H](C(*)=O)CC2)CC1] |
| (D-gE) | 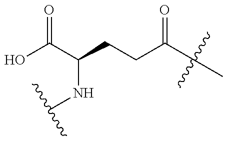 [SMILES: *N[C@H](CCC(*)=O)C(=O)O] |
| (D-gE)3 | 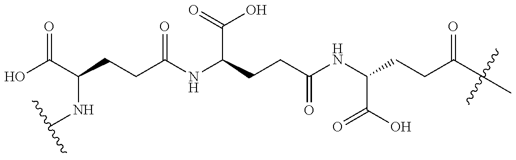 [SMILES: *N[C@H](CCC(=O)N[C@H](CCC(=O)N[C@H](CCC(*)=O)C(=O)O)C(=O)O)C(=O)O] |
| (gE)3 | 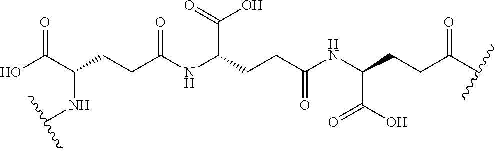 [SMILES: *N[C@@H](CCC(=O)N[C@@H](CCC(=O)N[C@@H](CCC(*)=O)C(=O)O)C(=O)O)C(=O)O] |
| (Sar)5 | 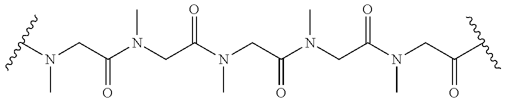 [SMILES: *C(=O)CN(C)C(=O)CN(C)C(=O)CN(C)C(=O)CN(C)C(=O)CN(*)C] |
| 1Me-Trp (1Me-W) | 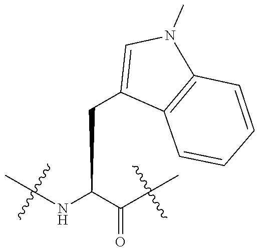 [SMILES: *N[C@@H](Cc1cn(C)c2ccccc12)C(*)=O] |
| 1Nal | 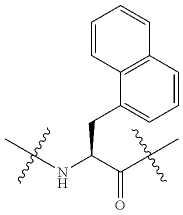 [SMILES: *N[C@@H](Cc1cccc2ccccc12)C(*)=O] |
| 2CF3-Phe (2CF3-F) | 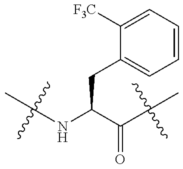 [SMILES: *N[C@@H](Cc1ccccc1C(F)(F)F)C(*)=O] |
| 2Nal | 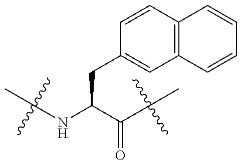 [SMILES: *N[C@@H](Cc1ccc2ccccc2c1)C(*)=O] |
| 2PhEt-Ala (2PhEt-A) | 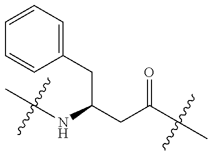 [SMILES: *N[C@H](CC(*)=O)Cc1ccccc1] |
| 3-(1- morpholinyl- Ala | 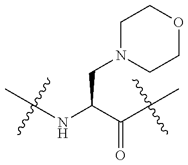 [SMILES: *N[C@@H](CN1CCOCC1)C(*)=O] |
| 3-(4- piperidinyl)- Ala | 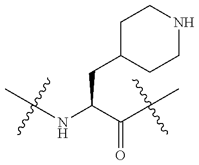 [SMILES: *N[C@@H](CC1CCNCC1)C(*)=O] |
| 3Me2-Aze | 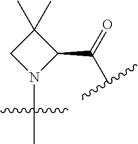 [SMILES: *C(=O)[C@H]1N(*)CC1(C)C] |
| 3Pya | 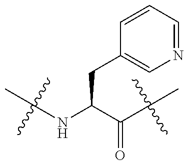 [SMILES: *N[C@@H](Cc1cccnc1)C(*)=O] |
| 4CF3-Phe (4CF3-F) | 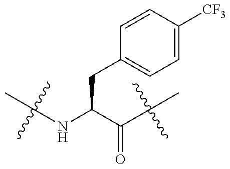 [SMILES: *N[C@@H](Cc1ccc(C(F)(F)F)cc1)C(*)=O] |
| 4F-Phe (4Fluoro-F) | 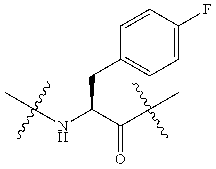 [SMILES: *N[C@@H](Cc1ccc(F)cc1)C(*)=O] |
| 4Pya | 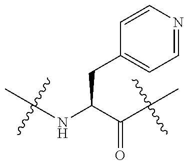 [SMILES: *N[C@@H](Cc1ccncc1)C(*)=O] |
| 5,5-diMe- Pro (5,5- diMe-P) | 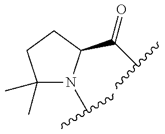 [SMILES: *C(=O)[C@@H]1CCC(C)(C)N1*] |
| 5Fluoro-Trp (5Fluoro-W) | 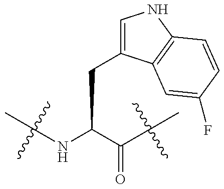 [SMILES: *N[C@@H](Cc1c[nH]c2ccc(F)cc12)C(*)=O] |
| 5OH-Trp (5OH-W) | 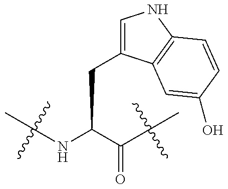 [SMILES: *N[C@@H](Cc1c[nH]c2ccc(O)cc12)C(*)=O] |
| 5OMe-Trp (5OMe-W) | 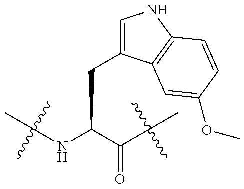 [SMILES: *N[C@@H](Cc1c[nH]c2ccc(OC)cc12)C(*)=O] |
| 5Qui | 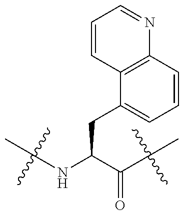 [SMILES: *N[C@@H](Cc1cccc2ncccc12)C(*)=O] |
| 7Aza-Trp (7Aza-W) | 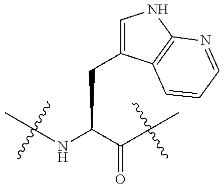 [SMILES: *N[C@@H](Cc1c[nH]c2ncccc12)C(*)=O] |
| 7Cl-Trp (7Cl-W) | 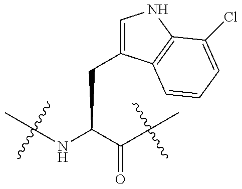 [SMILES: *N[C@@H](Cc1c[nH]c2c(Cl)cccc12)C(*)=O] |
| 7MeO-Trp (7MeO-W) | 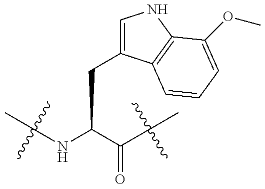 [SMILES: *N[C@@H](Cc1c[nH]c2c(OC)cccc12)C(*)=O] |
| 7Me-Trp (7Me-W) | 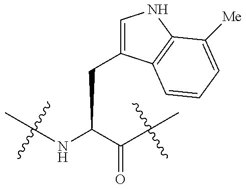 [SMILES: *N[C@@H](Cc1c[nH]c2c(C)cccc12)C(*)=O] |
| ACl | 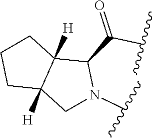 [SMILES: *C(=O)[C@@H]1[C@H]2CCC[C@H]2CN1*] |
| Ahx | 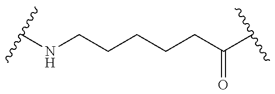 [SMILES: *NCCCCCC(*)=O] |
| Allo-Ile | 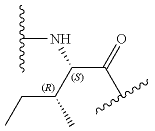 |
| AMBX | 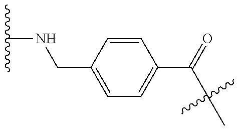 [SMILES: *NCc1ccc(C(*)=O)cc1] |
| aMe-Aze | 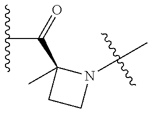 [SMILES: *C(=O)[C@]1(C)CCN1*] |
| alpha- Me-Asp (aMe-D) | 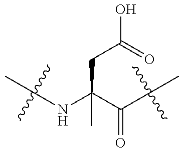 [SMILES: *N[C@](C)(CC(=O)O)C(*)=O] |
| alpha- Me-Ile (aMe-I) | 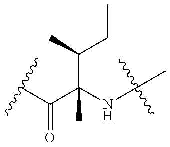 [SMILES: *N[C@](C)(C(*)=O)[C@@H](C)CC] |
| alpha- Me-Pro (aMe-P) | 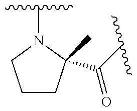 [SMILES: *C(=O)[C@]1(C)CCCN1*] |
| alpha- Me-Thr (aMe-T) | 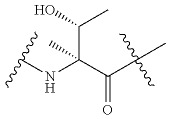 [SMILES: *N[C@](C)(C(*)=O)[C@@H](C)O] |
| alpha- Me-Trp (aMe-W) | 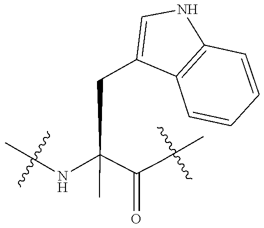 [SMILES: *CC(=O)[C@@](C)(Cc1c[nH]c2ccccc12)N*] |
| Aze | 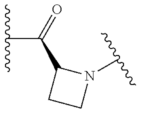 |
| BIP | 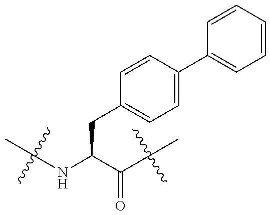 [SMILES: *N[C@@H](Cc1ccc(-c2ccccc2)cc1)C(*)=O] |
| C12OH | 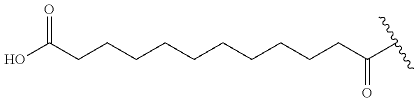 [SMILES: *C(=O)CCCCCCCCCCC(=O)O] |
| C14 | 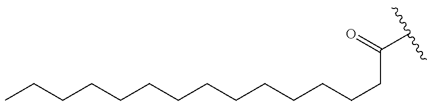 [SMILES: *C(=O)CCCCCCCCCCCCCC] |
| C15 | 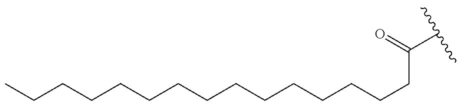 [SMILES: *C(=O)CCCCCCCCCCCCCCC] |
| C15-(L-gE)- PEG12 | 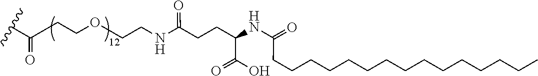 |
| COC16 | 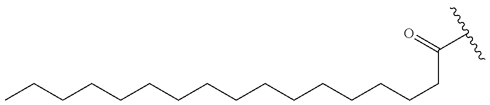 [SMILES: *C(=O)CCCCCCCCCCCCCCCC] |
| CBA | 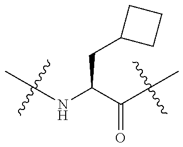 [SMILES: *N[C@@H](CC1CCC1)C(*)=O] |
| CHA | 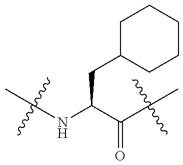 [SMILES: *N[C@@H](CC1CCCCC1)C(*)=O] |
| Chg | 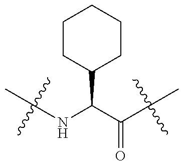 [SMILES: *N[C@H](C(*)=O)C1CCCCC1] |
| cis4Fluoro- Pro (cis4Fluoro- P) | 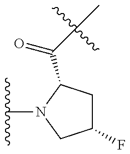 [SMILES: *C(=O)[C@@H]1C[C@H](F)CN1*] |
| cis4NH2-Pro (cis4NH2-P) | 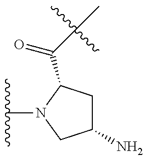 [SMILES: *C(=O)[C@@H]1C[C@H](N)CN1*] |
| cis4OH-Pro (cis4OH-P) | 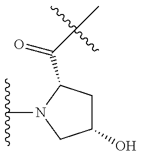 [SMILES: *C(=O)[C@@H]1C[C@H](O)CN1*] |
| CyHex | 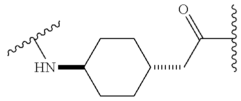 [SMILES: *N[C@H]1CC[C@H](CC(*)=O)CC1] |
| DAB-4- NHCOC5H11 | 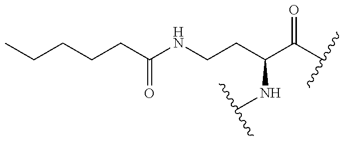 [SMILES: *N[C@@H](CCNC(=O)CCCCC)C(*)=O] |
| DAB-4- NHCOC7H15 | 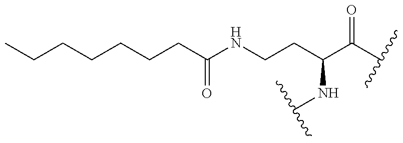 [SMILES: *N[C@@H](CCNC(=O)CCCCCCC)C(*)=O] |
| D-Ala | 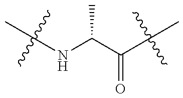 [SMILES: *N[C@H](C)C(*)=O] |
| D-Lys((D-Pro)3- PEG8-) | 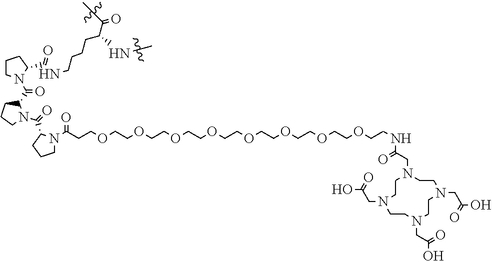 |
| D-Lys((D-Pro)- PEG8-DOTA) | 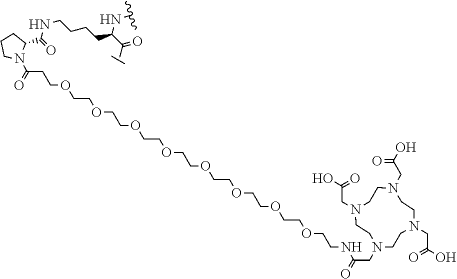 |
| D-Lys(Ahx- DOTA) | 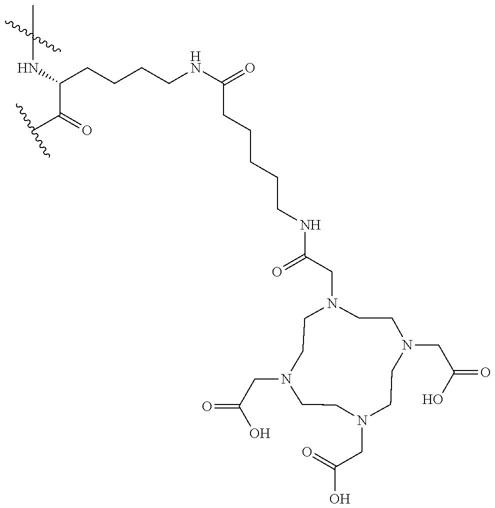 [SMILES: *N[C@H](CCCCNC(=O)CCCCCNC(=O)CN1CCN(CC(=O)O)CCN(CC(=O)O)CCN(CC(=O)O)CC1)C(*)=O] |
| D-Lys(Chx- DOTA) | 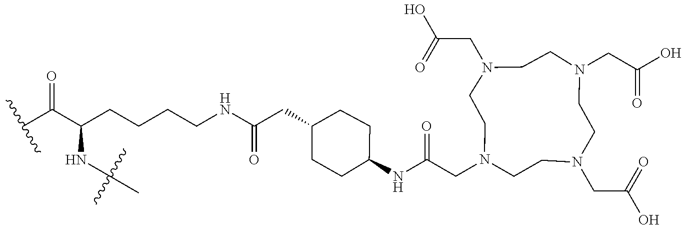 [SMILES: *N[C@H](CCCCNC(=O)C[C@H]1CC[C@H](NC(=O)CN2CCN(CC(=O)O)CCN(CC(=O)O)CCN(CC(=O)O)CC2)CC1)C(*)=O] |
| D-Lys(DOTA) | 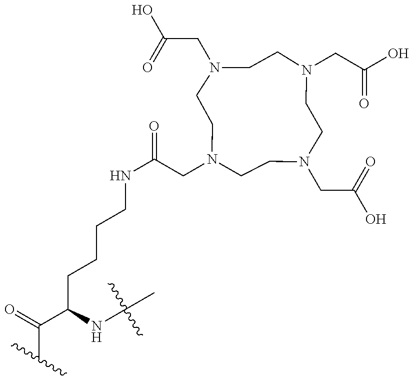 [SMILES: *N[C@H](CCCCNC(=O)CN1CCN(CC(=O)O)CCN(CC(=O)O)CCN(CC(=O)O)CC1)C(*)=O] |
| D-Lys(gE-DOTA) | 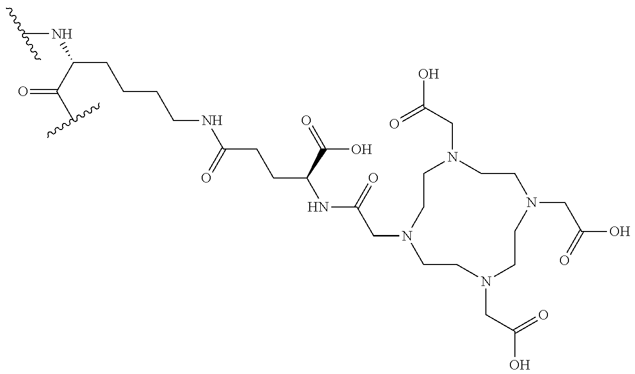 [SMILES: *N[C@H](CCCCNC(=O)CC[C@H](NC(=O)CN1CCN(CC(=O)O)CCN(CC(=O)O)CCN(CC(=O)O)CC1)C(=O)O)C(*)=O] |
| D-Lys(gE-gE-gE- DOTA) | 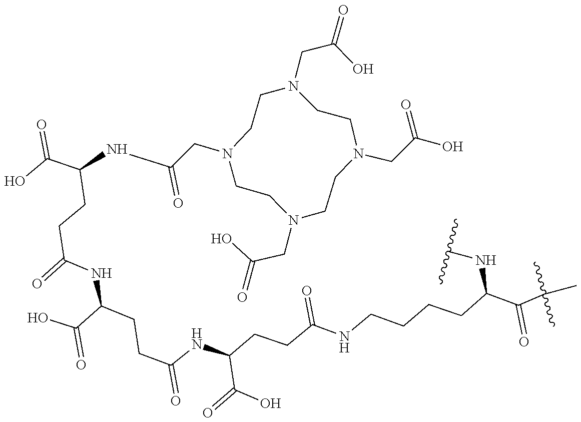 [SMILES: *N[C@H](CCCCNC(=O)CC[C@H](NC(=O)CC[C@H](NC(=O)CC[C@H](NC(=O)CN1CCN(CC(=O)O)CCN(CC(=O)O)CCN(CC(=O)O)CC1)C(=O)O)C(=O)O)C(=O)O)C(*)=O] |
| D-Lys(Gly-Gly- Ser-Gly-Gly-Ser- DOTA) | 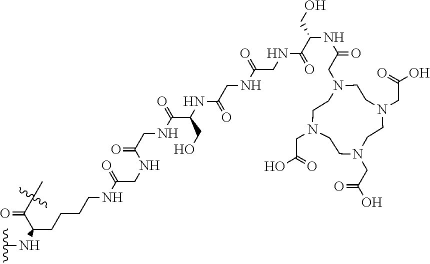 [SMILES: *N[C@H](CCCCNC(=O)CNC(=O)CNC(=O)[C@H](CO)NC(=O)CNC(=O)CNC(=O)[C@H](CO)NC(=O)CN1CCN(CC(=O)O)CCN(CC(=O)O)CCN(CC(=O)O)CC1)C(*)=O] |
| D-Lys(Gly-Gly- TTDS-DOTA) | 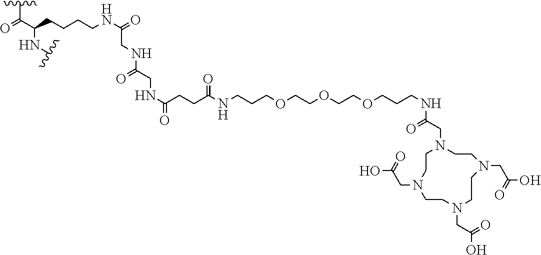 [SMILES: *N[C@H](CCCCNC(=O)CNC(=O)CNC(=O)CCC(=O)NCCCOCCOCCOCCCNC(=O)CN1CCN(CC(=O)O)CCN(CC(=O)O)CCN(CC(=O)O)CC1)C(*)=O] |
| D-Lys(Gly-Ser- Gly-Ser-Gly-Ser- DOTA) | 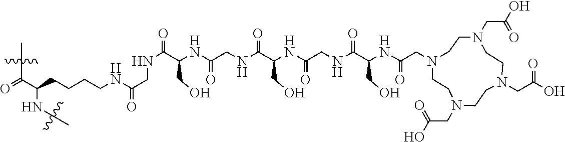 [SMILES: *N[C@H](CCCCNC(=O)CNC(=O)[C@H](CO)NC(=O)CNC(=O)[C@H](CO)NC(=O)CNC(=O)[C@H](CO)NC(=O)CN1CCN(CC(=O)O)CCN(CC(=O)O)CCN(CC(=O)O)CC1)C(*)=O] |
| D-Lys(PEG4- DOTA) | 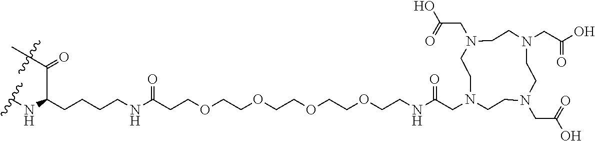 [SMILES: *N[C@H](CCCCNC(=O)CCOCCOCCOCCOCCNC(=O)CN1CCN(CC(=O)O)CCN(CC(=O)O)CCN(CC(=O)O)CC1)C(*)=O] |
| D-Lys(PEG8- COCH2Ph-4Br) | 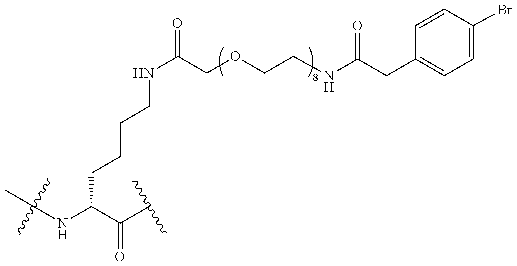 |
| D-Lys(PEG8- COCH2Ph-4I) | 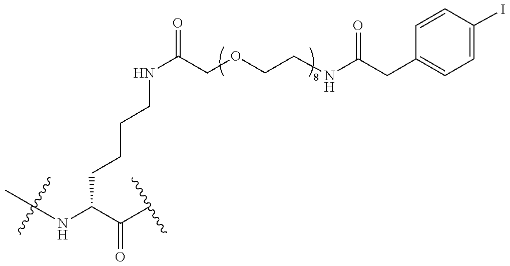 |
| D-Lys(PEG8- DOTA) | 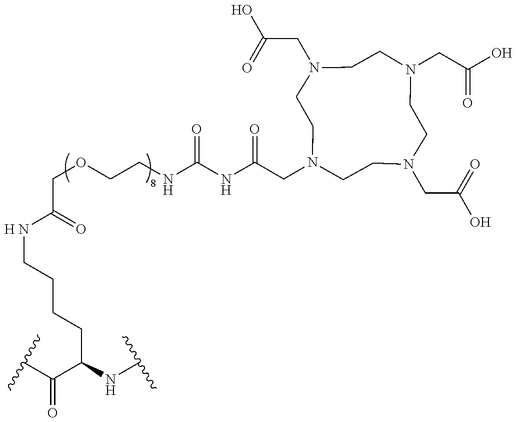 |
| D-Lys(SP6- PEG8-DOTA) | 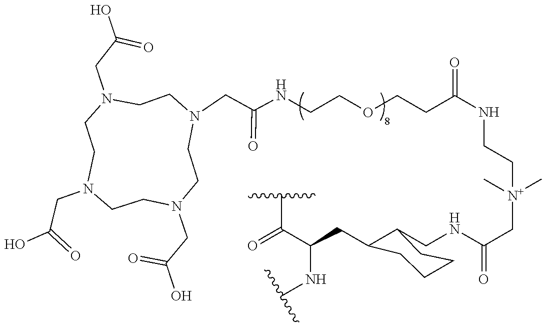 |
| D-Lys[PEG8-D- Lys(DOTA)- PEG12- COCH2Ph-4Br] | 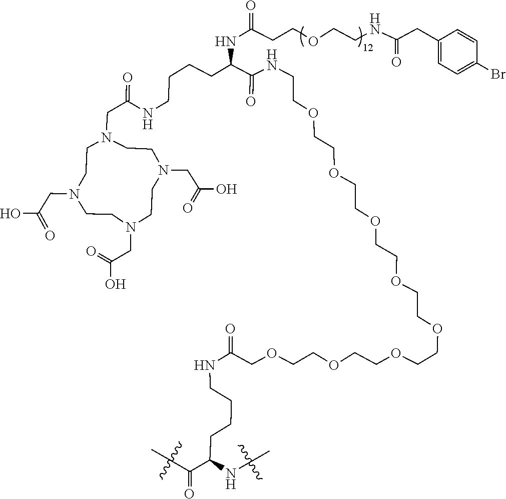 |
| D-Lys[PEG8-D- Lys(DOTA)- PEG24- COCH2Ph-4Br] | 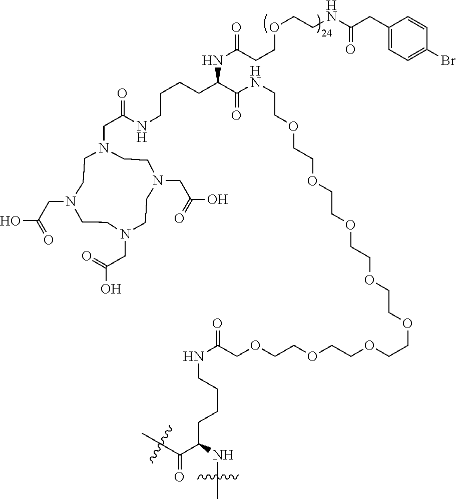 |
| D-Lys[PEG8-D- Lys(DOTA)- PEG8-COCH2Ph- 4Br] | 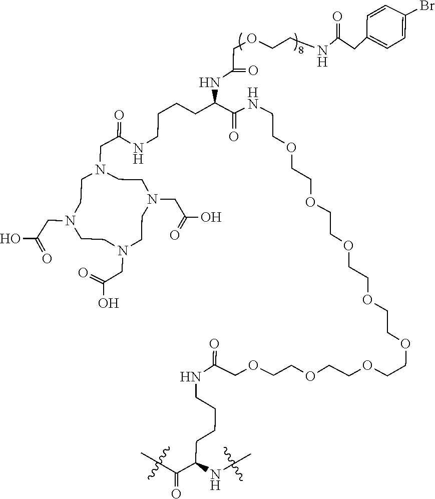 |
| D-Nle | 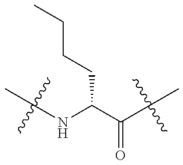 [SMILES: *N[C@H](CCCC)C(*)=O] |
| D-Pro (dPro) | 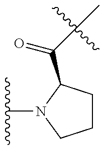 [SMILES: *C(=O)[C@H]1CCCN1*] |
| Env | 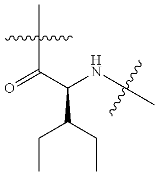 [SMILES: *N[C@H](C(*)=O)C(CC)CC] |
| gE | 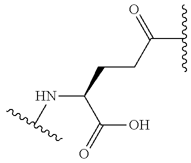 [SMILES: *N[C@@H](CCC(*)=O)C(=O)O] |
| Glu[gLys(OH)- Val-Met-AmBz- DOTA] | 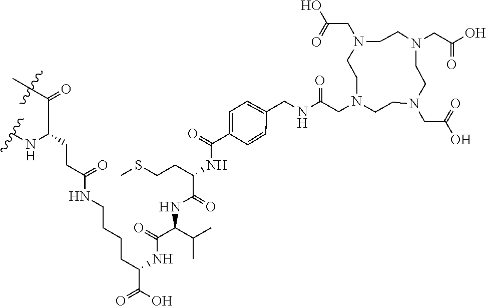 [SMILES: *N[C@@H](CCC(=O)NCCCC[C@H](NC(=O)[C@@H](NC(=O)[C@H](CCSC)NC(=O)c1ccc(CNC(=O)CN2CCN(CC(=O)O)CCN(CC(=O)O)CCN(CC(=O)O)CC2)cc1)C(C)C)C(=O)O)C(*)=O] |
| Lys(C12) | 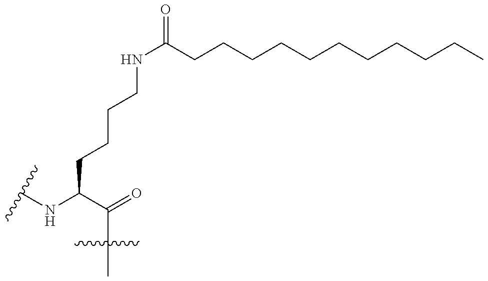 [SMILES: *N[C@@H](CCCCNC(=O)CCCCCCCCCCC)C(*)=O] |
| Lys(C14) | 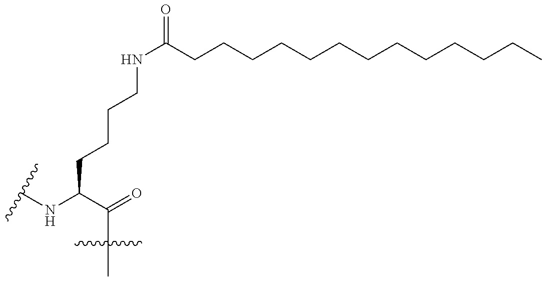 [SMILES: *N[C@@H](CCCCNC(=O)CCCCCCCCCCCCC)C(*)=O] |
| Lys(C16) | 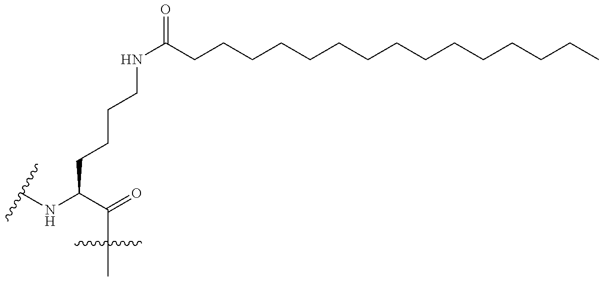 [SMILES: *N[C@@H](CCCCNC(=O)CCCCCCCCCCCCCCC)C(*)=O] |
| Lys(Me)3 | 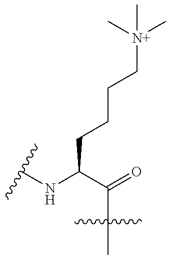 [SMILES: *N[C@@H](CCCC[N+](C)(C)C)C(*)=O] |
| Lys(DOTA) | 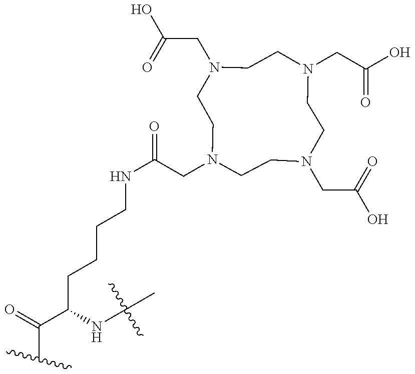 [SMILES: *N[C@@H](CCCCNC(=O)CN1CCN(CC(=O)O)CCN(CC(=O)O)CCN(CC(=O)O)CC1)C(*)=O] |
| Lys(Chx-DOTA) | 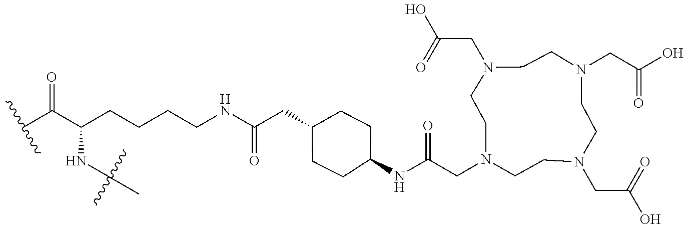 [SMILES: *N[C@@H](CCCCNC(=O)C[C@H]1CC[C@H](NC(=O)CN2CCN(CC(=O)O)CCN(CC(=O)O)CCN(CC(=O)O)CC2)CC1)C(*)=O] |
| Lys(Gly-Gly- TTDS-DOTA) | 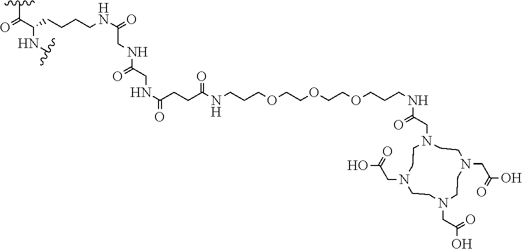 [SMILES: *N[C@@H](CCCCNC(=O)CNC(=O)CNC(=O)CCC(=O)NCCCOCCOCCOCCCNC(=O)CN1CCN(CC(=O)O)CCN(CC(=O)O)CCN(CC(=O)O)CC1)C(*)=O] |
| Lys(Gly-Ser-Gly- Ser-Gly-Ser- DOTA) | 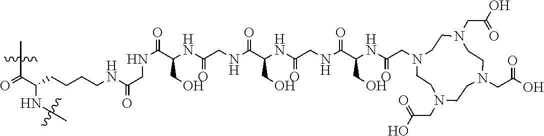 [SMILES: *N[C@@H](CCCCNC(=O)CNC(=O)[C@H](CO)NC(=O)CNC(=O)[C@H](CO)NC(=O)CNC(=O)[C@H](CO)NC(=O)CN1CCN(CC(=O)O)CCN(CC(=O)O)CCN(CC(=O)O)CC1)C(*)=O] |
| MAF | 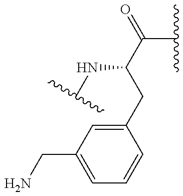 [SMILES: *N[C@@H](Cc1cccc(CN)c1)C(*)=O] |
| Nle | 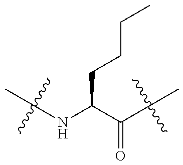 [SMILES: *N[C@@H](CCCC)C(*)=O] |
| NMe-Ala (NMeA) | 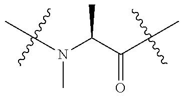 [SMILES: *C(=O)[C@H](C)N(*)C] |
| NMe-Asp (NMeD) | 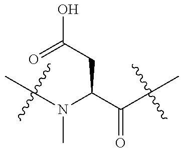 [SMILES: *C(=O)[C@H](CC(=O)O)N(*)C] |
| NMe-His (NMeH) | 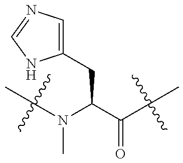 [SMILES: *C(=O)[C@H](Cc1cnc[nH]1)N(*)C] |
| NMeK(DOTA) | 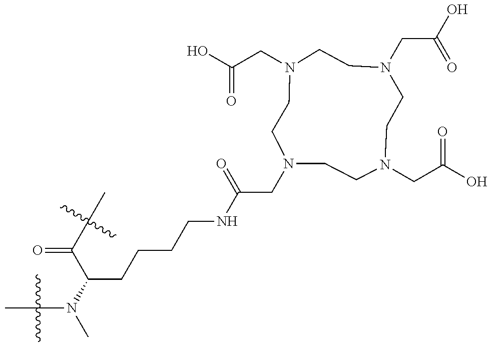 [SMILES: *C(=O)[C@H](CCCCNC(=O)CN1CCN(CC(=O)O)CCN(CC(=O)O)CCN(CC(=O)O)CC1)N(*)C] |
| NMe-Leu (NMeL) | 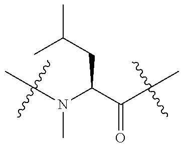 [SMILES: *C(=O)[C@H](CC(C)C)N(*)C] |
| NMe-Asn (NMeN) | 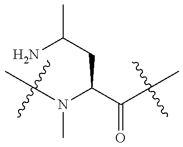 [SMILES: *C(=O)[C@H](CC(C)N)N(*)C] |
| NMe-Nle | 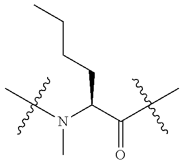 [SMILES: *C(=O)[C@H](CCCC)N(*)C] |
| NMe-Ser (NMeS) | 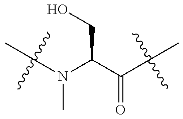 [SMILES: *C(=O)[C@H](CO)N(*)C] |
| NMe-Thr (NMeT) | 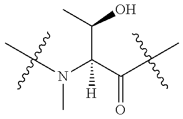 [SMILES: *C(=O)[C@H]([C@@H](C)O)N(*)C] |
| NMe-tBuAla | 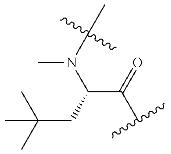 [SMILES: *C(=O)[C@H](CC(C)(C)C)N(*)C] |
| NMe-Trp (NMeW) | 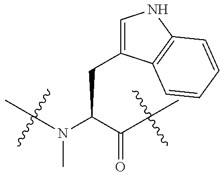 [SMILES: *C(=O)[C@H](Cc1c[nH]c2ccccc12)N(*)C] |
| OAF | 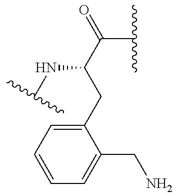 [SMILES: *N[C@@H](Cc1ccccc1CN)C(*)=O] |
| Orn | 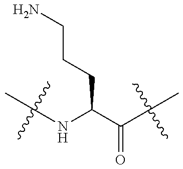 [SMILES: *N[C@@H](CCCN)C(*)=O] |
| PAF | 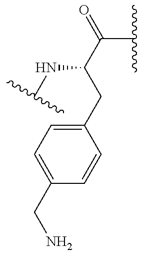 [SMILES: *N[C@@H](Cc1ccc(CN)cc1)C(*)=O] |
| PEG4 | 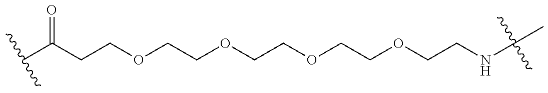 [SMILES: *NCCOCCOCCOCCOCCC(*)=O] |
| PEG8 | 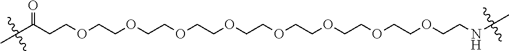 [SMILES: *NCCOCCOCCOCCOCCOCCOCCOCCOCCC(*)=O] |
| Pip | 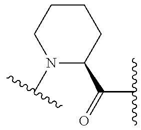 [SMILES: *C(=O)[C@@H]1CCCCN1*] |
| R-3Me-Aze | 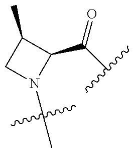 [SMILES: *C(=O)[C@@H]1[C@H](C)CN1*] |
| Sar | 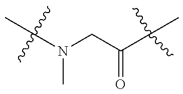 [SMILES: *C(=O)CN(*)C] |
| SP6 | 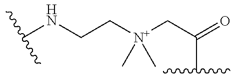 [SMILES: *NCC[N+](C)(C)CC(*)=O] |
| Tbg | 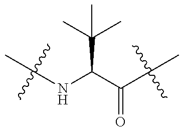 [SMILES: *N[C@H](C(*)=O)C(C)(C)C] |
| t-Bu-Ala | 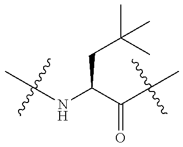 [SMILES: *N[C@@H](CC(C)(C)C)C(*)=O] |
| THPG | 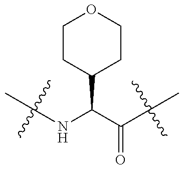 [SMILES: *N[C@H](C(*)=O)C1CCOCC1] |
| trans4Fluoro-Pro (trans4Fluoro-P) | 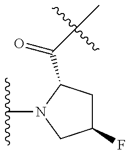 [SMILES: *C(=O)[C@@H]1C[C@@H](F)CN1*] |
| trans4NH2-Pro (trans4NH2-P) | 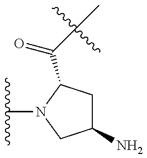 [SMILES: *C(=O)[C@@H]1C[C@@H](N)CN1*] |
| trans4OH-Pro (trans4OH-P) | 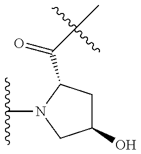 [SMILES: *C(=O)[C@@H]1C[C@@H](O)CN1*] |
| TTDS-Gly-Gly | 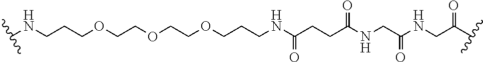 [SMILES: *NCCCOCCOCCOCCCNC(=O)CCC(=O)NCC(=O)NCC(*)=O] |
| α-tert-amylGly | 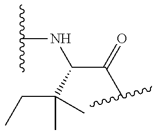 [SMILES: *N[C@H](C(*)=O)C(C)(C)CC] |
| DapN3 | 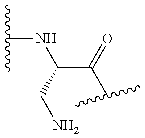 [SMILES: *N[C@@H](CN)C(*)=O] |
| Pra | 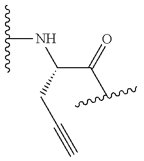 [SMILES: *N[C@@H](CC#C)C(*)=O] |
| Dap | 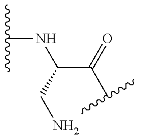 [SMILES: *N[C@@H](CN)C(*)=O] |
| L-2,4-Dab | 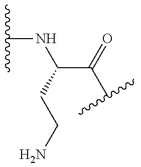 [SMILES: *N[C@@H](CCN)C(*)=O] |
| AllylG | 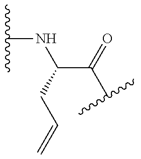 [SMILES: *N[C@@H](CC=C)C(*)=O] |
| Lys(PEG24- [gE(C16)]-OH) | 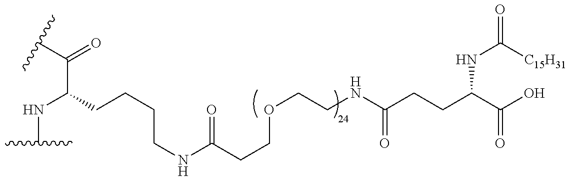 |
| Macrodipa | 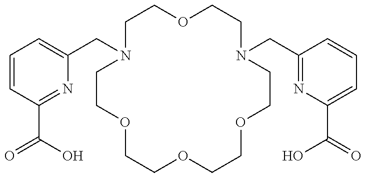 [SMILES: O=C(O)c1cccc(CN2CCOCCOCCOCCN(Cc3cccc(C(=O)O)n3)CCOCC2)n1] |
| D-Lys(Ac) | 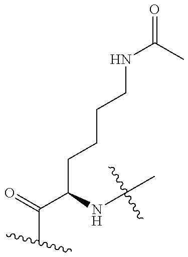 [SMILES: *N[C@H](CCCCNC(C)=O)C(*)=O] |
| C14OH | 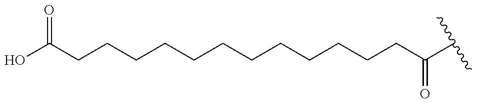 [SMILES: *C(=O)CCCCCCCCCCCCC(=O)O] |
| C16OH | 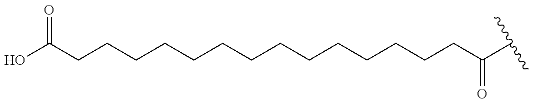 [SMILES: *C(=O)CCCCCCCCCCCCCCC(=O)O] |
| alpha-Me-Cys | 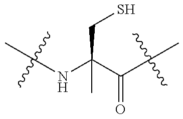 [SMILES: *N[C@](C)(CS)C(*)=O] |
| NMe-Cys | 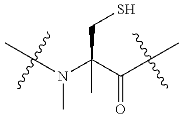 [SMILES: *C(=O)[C@@](C)(CS)N(*)C] |
| D-Cys | 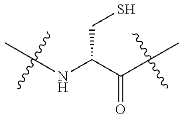 [SMILES: *N[C@H](CS)C(*)=O] |
| Pip(CH₂CO₂H)Ala | 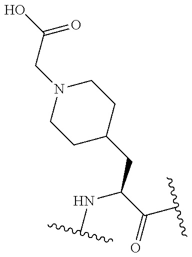 [SMILES: *N[C@@H](CC1CCN(CC(=O)O)CC1)C(*)=O] |
| Pip(PEGNMe₃)Ala | 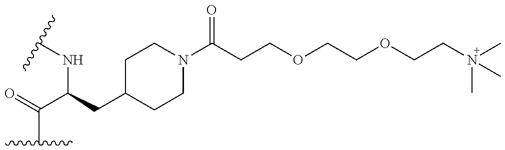 |
| Pip(GAE- DOTA)Ala | 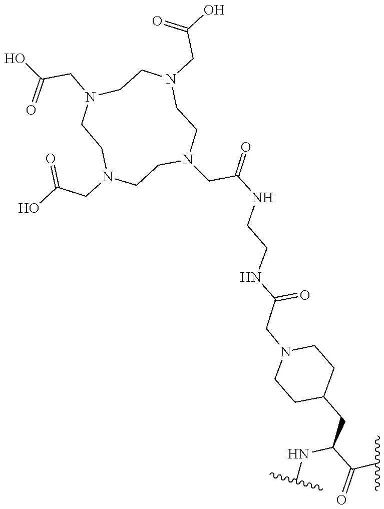 [SMILES: *N[C@@H](CC1CCN(CC(=O)NCCNC(=O)CN2CCN(CC(=O)O)CCN(CC(=O)O)CCN(CC(=O)O)CC2)CC1)C(*)=O] |

### V. Equivalents and Scope

While various disclosure embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The disclosure includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Reagents Abbreviations

ACN (or MeCN): Acetonitrile
Ac₂O: Acetic anhydride
AcOH: Acetic acid
Boc: Tert-butoxy-carbonyl
DCM: Dichloromethane
DIC: N,N′-Diisopropylcarbodiimide
DIPEA: N,N-Diisopropylethylamine
DMF: N,N-Dimethylformamide
DMSO: Dimethyl sulfoxide
Et₂O: Diethyl ether
Fmoc: Fluorenylmethyloxycarbonyl
HATU: 2-(3H-[1,2,3]triazolo[4,5-b]pyridin-3-yl)-1,1,3,3-tetramethylisouronium
HOBt: 1-Hydroxybenzotriazole
MeOH: Methanol
Mpe: 3-Methylpentan-3-yl
MTBE: Methyl-tert-butyl-ether
NMP: N-Methyl-2-Pyrrolidone
Oxyma (or OxymaPure): Ethyl cyanohydroxyiminoacetate
Dde: 1-(4,4-dimethyl-2,6-dioxocyclohexylidene)ethyl
TFA: Trifluoroacetic acid
TIPS: Triisopropylsilane
Cp*RuCl(PPh₃)₂: Chloro(pentamethylcyclopentadienyl)bis(triphenylphosphine)ruthenium(II) Grubbs2: (1,3-Dimesitylimidazolidin-2-ylidene)(tricyclohexylphosphine)benzylidene ruthenium dichloride

### Amino Acids and Building Blocks

Na-(((9H-fluoren-9-yl)methoxy)carbonyl)-1-(tert-butoxycarbonyl)-L-tryptophan (W)
(((9H-fluoren-9-yl)methoxy)carbonyl)-L-threonine (T)
(((9H-fluoren-9-yl)methoxy)carbonyl)-L-isoleucine (I)
N-(((9H-fluoren-9-yl)methoxy)carbonyl)-S-trityl-L-cysteine (C)
(S)-2-((((9H-fluoren-9-yl)methoxy)carbonyl)amino)-3-(tritylamino)propanoic acid (N)
Na-(((9H-fluoren-9-yl)methoxy)carbonyl)-Np-(tert-butoxycarbonyl)-L-histidine (H)
(S)-2-((((9H-fluoren-9-yl)methoxy)carbonyl)amino)-4-((3-methylpentan-3-yl)oxy)-4-oxobutanoic acid (D)
(((9H-fluoren-9-yl)methoxy)carbonyl)-L-proline (P)
N2-(((9H-fluoren-9-yl)methoxy)carbonyl)-N6-(1-(4,4-dimethyl-2,6-dioxocyclohexylidene)ethyl)-L-lysine (K(Dde))
N2-(((9H-fluoren-9-yl)methoxy)carbonyl)-N6-(1-(4,4-dimethyl-2,6-dioxocyclohexylidene)ethyl)-D-lysine (D-Lys(Dde))
N2-(((9H-fluoren-9-yl)methoxy)carbonyl)-N6-(tert-butoxycarbonyl)-D-lysine (D-Lys(Boc))
(S)-2-((((9H-fluoren-9-yl)methoxy)carbonyl)amino)-3-(naphthalen-2-yl)propanoic acid (2Nal)
2-(4,7,10-tris(2-(tert-butoxy)-2-oxoethyl)-1,4,7,10-tetraazacyclododecan-1-yl)acetic acid (DOTA(tBu)₃)
2,2′,2″-(10-(2-((2,5-dioxopyrrolidin-1-yl)oxy)-2-oxoethyl)-1,4,7,10-tetraazacyclododecane-1,4,7-triyl)triacetic acid (DOTA-NHS)
(S)-4-((((9H-fluoren-9-yl)methoxy)carbonyl)amino)-6,6-dimethyl-5-oxoheptanoic acid (gE)
3-((tert-butoxycarbonyl)amino)propanoic acid (Boc-betaAla)
(S)-2-((((9H-fluoren-9-yl)methoxy)carbonyl)amino)pent-4-enoic acid (AIlyIG)
(S)-2-((((9H-fluoren-9-yl)methoxy)carbonyl)amino)-3-azidopropanoic acid (DapN₃)
(S)-2-((((9H-fluoren-9-yl)methoxy)carbonyl)amino)pent-4-ynoic acid (Pra)

### Example 1. Targeting Construct Preparation

A targeting construct is prepared by combining a targeting moiety with a cargo. The targeting moiety incorporates peptide sequences specific for a cancer cell antigen selected from DLL3. The cargo includes a radioactive agent that includes a radioisotope. The targeting moiety and cargo are combined using a linker.

In some embodiments, DOTA chelators can be attached to the cyclic peptide targeting moieties according to the following general method.

General Peptide Synthesis for SEQ ID NO: 3-89 (0.1 mmol Scale):

Peptides were synthesized using the CEM Liberty Blue microwave peptide synthesizer. Standard Fmoc chemistry and couplings were used with ProTide Rink Amide resin (0.19 g/mmol).

Step 1: Deprotection: 20% piperidine in DMF (3 mL, 75 equiv.) was added to the resin and the mixture was heated to 90° C. for 1 minute. The resin was then washed with 4 mL of DMF 4 times.

Step 2: Double Coupling: To the microwave reaction vessel was added Fmoc-protected amino acid (5 equiv.) in DMF (0.2M), DIC (10 equiv.) in DMF (1M), and OxymaPure (5 equiv.) in DMF (1M). The microwave reaction vessel is then heated to 90° C. for 4 minutes and then drained. To the reaction vessel was added Fmoc-protected amino acid (5 equiv.) in DMF (0.2M), DIC (10 equiv.) in DMF (1M), and OxymaPure (5 equiv.) in DMF (1M). The microwave reaction vessel is then heated to 90° C. for 4 minutes and then drained.

Step 3: Steps 1 and 2 were repeated for the remaining amino acids according to Table 4 (Coupling Nos. 2-12).

Step 4: DOTA Single Coupling (Coupling No. 13): Normal deprotection protocol used (Step 1) to remove the Fmoc group on AA No. 12. To the microwave reaction vessel was added 2-(4,7,10-tris(2-(tert-butoxy)-2-oxoethyl)-1,4,7,10-tetraazacyclododecan-1-yl)acetic acid (5 equiv.) in DMF (0.2M), DIC (10 equiv.) in DMF (1M), and OxymaPure (5 equiv.) in DMF (1M). The microwave reaction vessel is then heated to 50° C. for 10 minutes and then drained.

Step 5: Capping (Coupling No. 14; if no DOTA chelator attached): Normal deprotection protocol used (Step 1) to remove the Fmoc group on AA No. 12. After the 4 DMF washes, 2.5 mL of 10% Ac2O in DMF was added to the microwave reaction vessel and then heated to 65° C. for 2 minutes. The resin was then washed with 4 mL of DMF 4 times.

TABLE 4

| Coupling | | |
|---|---|---|
| Nos. | Materials | Coupling reagents |
| 1 | Fmoc-AA No. 1 (5 equiv.) | DIC (10 equiv.) and OxymaPure (5 |
| | | equiv.) |
| 2 | Fmoc-AA No. 2 (5 equiv.) | DIC (10 equiv.) and OxymaPure (5 |
| | | equiv.) |
| 3 | Fmoc-AA No. 3 (5 equiv.) | DIC (10 equiv.) and OxymaPure (5 |
| | | equiv.) |
| 4 | Fmoc-AA No. 4 (5 equiv.) | DIC (10 equiv.) and OxymaPure (5 |
| | | equiv.) |
| 5 | Fmoc-AA No. 5 (5 equiv.) | DIC (10 equiv.) and OxymaPure (5 |
| | | equiv.) |
| 6 | Fmoc-AA No. 6 (5 equiv.) | DIC (10 equiv.) and OxymaPure (5 |
| | | equiv.) |
| 7 | Fmoc-AA No. 7 (5 equiv.) | DIC (10 equiv.) and OxymaPure (5 |
| | | equiv.) |
| 8 | Fmoc-AA No. 8 (5 equiv.) | DIC (10 equiv.) and OxymaPure (5 |
| | | equiv.) |
| 9 | Fmoc-AA No. 9 (5 equiv.) | DIC (10 equiv.) and OxymaPure (5 |
| | | equiv.) |
| 10 | Fmoc-AA No. 10 (5 equiv.) | DIC (10 equiv.) and OxymaPure (5 |
| | | equiv.) |
| 11 | Fmoc-AA No. 11 (5 equiv.) | DIC (10 equiv.) and OxymaPure (5 |
| | | equiv.) |
| 12 | Fmoc-AA No. 12 (5 equiv.) | DIC (10 equiv.) and OxymaPure (5 |
| | | equiv.) |
| 13 | 2-(4,7, 10-tris(2-(tert-butoxy)-2- | DIC (10 equiv.) and OxymaPure (5 |
| | oxoethyl)-1,4,7,10- | equiv.) |
| | tetraazacyclododecan-1-yl)acetic | |
| | acid (5.00 equiv.) | |
| 14 (capping) | 10% Ac2O in DMF | N/A |

### Peptide Cleavage, Cyclization and Purification:

Using the OEM Razor peptide cleavage system, the peptide resin was transferred to a fritted cleavage tube, and 8 mL of cleavage solution (TFA/EDT/H₂O/TIS, 94/2.5/2.5/1, v/v/v/v) was added to each tube. The peptide resin in cleavage solution was heated to 40° C. for 40 minutes.

Each tube is filtered, and the filtrate is collected in centrifuge tubes.

The peptide is then precipitated with cold Ether (45 mL). The precipitated peptides are then placed on to a centrifuge and allowed to spin for at least 10 minutes. The ether is then decanted from each tube, leaving the peptide at the bottom. This Ether precipitation process is then repeated.

The peptide is then dissolved in 15 mL of MeCN/H₂O (1/1, v/v), and 0.1 M I₂ in MeOH was added dropwise until yellow color persisted. The mixture was stirred at 20° C. for 10 minutes. The reaction was quenched by adding 0.1 M Na₂S₂O₃ dropwise until the yellow color disappeared. The reaction solution is then concentrated under reduced pressure. The crude cyclized peptide is then dissolved in 2 mL of DMSO for purification.

The crude peptide in DMSO was purified by prep-HPLC (acidic condition, TFA) directly, followed by lyophilization to obtain the product.

Purification conditions:

Purification condition

| | |
|---|---|
| Dissolution | Dissolve in DMSO |
| condition | |
| Instrument | Waters |
| Mobile Phase | A: H₂O (0.1% TFA) |
| | B: CH₃CN (0.1% TFA) |
| Gradient | Variable Gradient - Determined by crude QC |
| Column | XBridge BEH C18 OBD Prep Column, 130 Å, 5 μm, |
| | 19 mm × 100 mm |
| Flow Rate | 24 mL/minute |
| Wavelength | 220/254 nm |
| Oven Tem. | Room temperature |

### Example 2. Synthetic Protocols

### Procedure D

### Example: Cpd No. 75

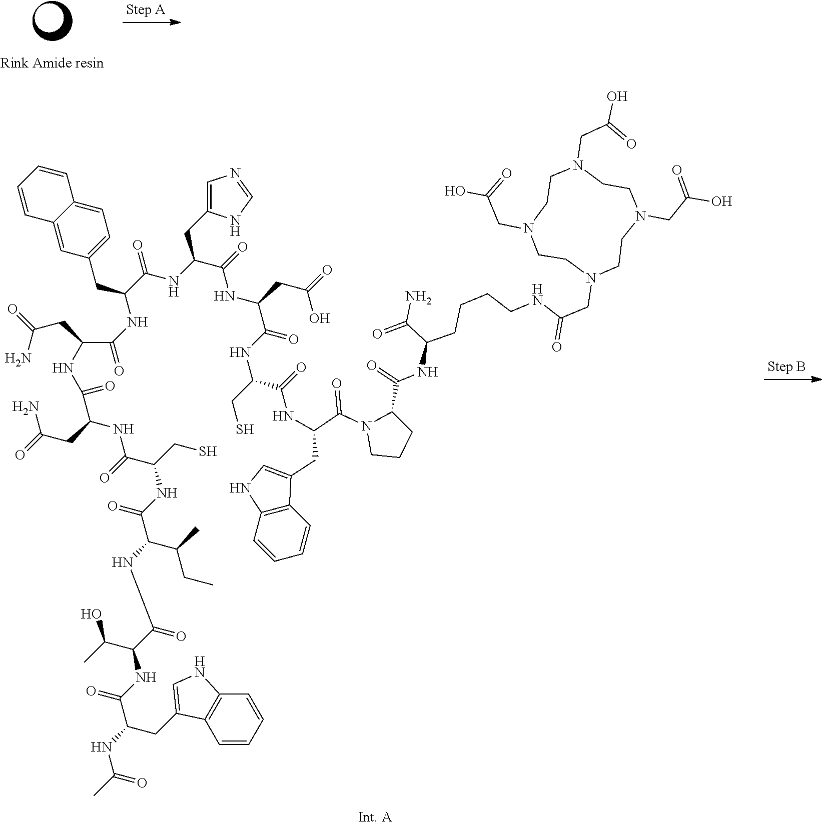 [SMILES: CC[C@H](C)[C@H](NC(=O)[C@@H](NC(=O)[C@H](Cc1c[nH]c2ccccc12)NC(C)=O)[C@@H](C)O)C(=O)N[C@@H](CS)C(=O)N[C@@H](CC(N)=O)C(=O)N[C@@H](CC(N)=O)C(=O)N[C@@H](Cc1ccc2ccccc2c1)C(=O)N[C@@H](Cc1cnc[nH]1)C(=O)N[C@@H](CC(=O)O)C(=O)N[C@@H](CS)C(=O)N[C@@H](Cc1c[nH]c2ccccc12)C(=O)N1CCC[C@H]1C(=O)N[C@H](CCCCNC(=O)CN1CCN(CC(=O)O)CCN(CC(=O)O)CCN(CC(=O)O)CC1)C(N)=O]

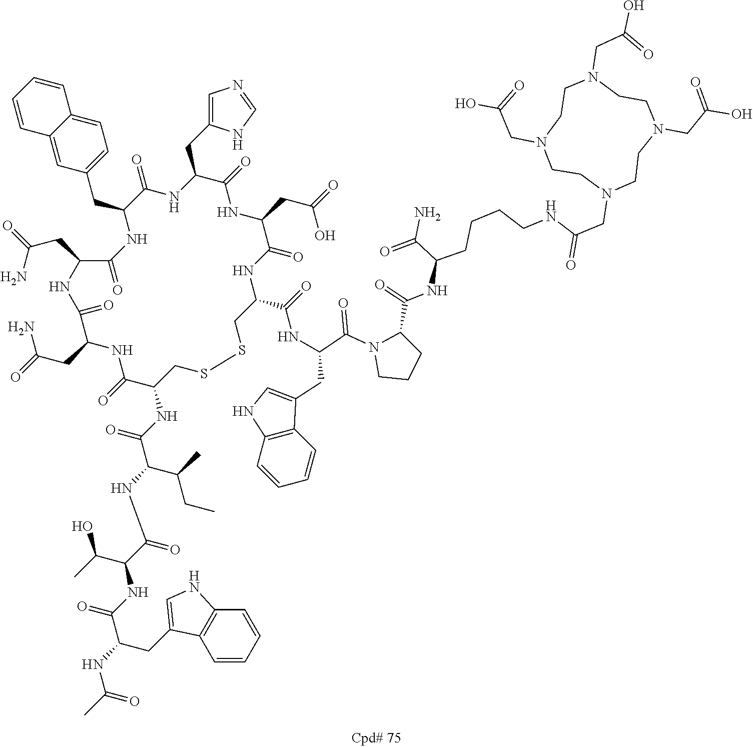 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

### Step A. Synthesis of Compound Int. A

The peptide was synthesized by standard Solid-phase Peptide Synthesis (SPPS) using Fmoc/t-Bu chemistry. The assembly was performed on a Rink-amide MBHA resin (250 umol, 100-200 Mesh; loading 0.42 mmol/g) on the OEM Liberty Blue microwave peptide synthesizer (CEM Inc.). During peptide assembly on solid phase, the side chain protecting groups were: tert-butyl for T; trityl for C and N; Boc for W and H; Mpe for D, Dde for D-Lys. All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1M solution of DIC in DMF and Oxyma solution 1M in DMF.

Double acylation reactions were performed for T², H⁸, N⁶ and W¹¹.

N-terminal acetylation was performed for 2 minutes at 65° C. under MW irradiation with a 10% v/v solution of Ac₂O in DMF.

At the end of the assembly, a 3% hydrazine monohydrate solution in DMF (50 mL) was percolated on the resin over 15 minutes to selectively remove the Dde protecting group from the D-Lys.

DOTA was incorporated manually using an equimolar solution of DOTA(tBu)₃, DIC and HOBt (3 Eq, 1:1:1) in NMP at room temperature and complete acylation was monitored by ninhydrin test.

At the end of the assembly the resin was washed with DMF, DCM, Et₂O. The peptide was cleaved from solid support using 50 mL of TFA solution (v/v: 87.5% TFA, 5% H₂O, 2.5% TIPS, 5% Phenol) for approximately 4 hours, at room temperature. The resin was then filtered and concentrated to about 10 mL and precipitated in cold MTBE (80 mL). After centrifugation, the peptide pellets were washed with fresh cold Et₂O to remove the organic scavengers. The process was repeated twice. Final pellets were dried, re-suInded in H₂O and ACN 1:1 and stirred overnight. Then lyophilized to afford crude Compound Int. A (376 mg, Yield: 70.2%). LCMS anal. calc. For C₉₈H₁₃₃N₂₅O₂₆S₂: 2141.4; found: 715.6 (M+3)³⁺

### Step B. Synthesis of Compound Cpd No. 75

Intermediate Compound A (Int. A) is dissolved in H₂O/ACN (1 mg/mL). Iodine (saturated solution in AcOH) was added until yellow color persisted. Stirred at room temperature for 5 min, then quenched with ascorbic acid and lyophilized. Crude peptide was purified by reverse-phase HPLC in two runs using preparative Waters XBridge C18 column (150×50 mm, 130 Å, 5 μm). Mobile phase A: H₂O+0.1% TFA, mobile phase B: ACN+0.1% TFA. The following gradient of eluent B was used: 20% B to 20% B over 5 min, to 35% B over 25 min, flow rate 80 mL/minute, wavelength 214 nm. Collected fractions were lyophilized to afford Cpd No. 75 (58 mg, Yield: 15.4%).

Cpd No. 262 was prepared using the methodology herein and the general procedure described in Cpd No. 75 with 2-Chlorotrityl Chloride resin instead of Rink Amide resin.

### Procedure F

### Example: Cpd No. 158

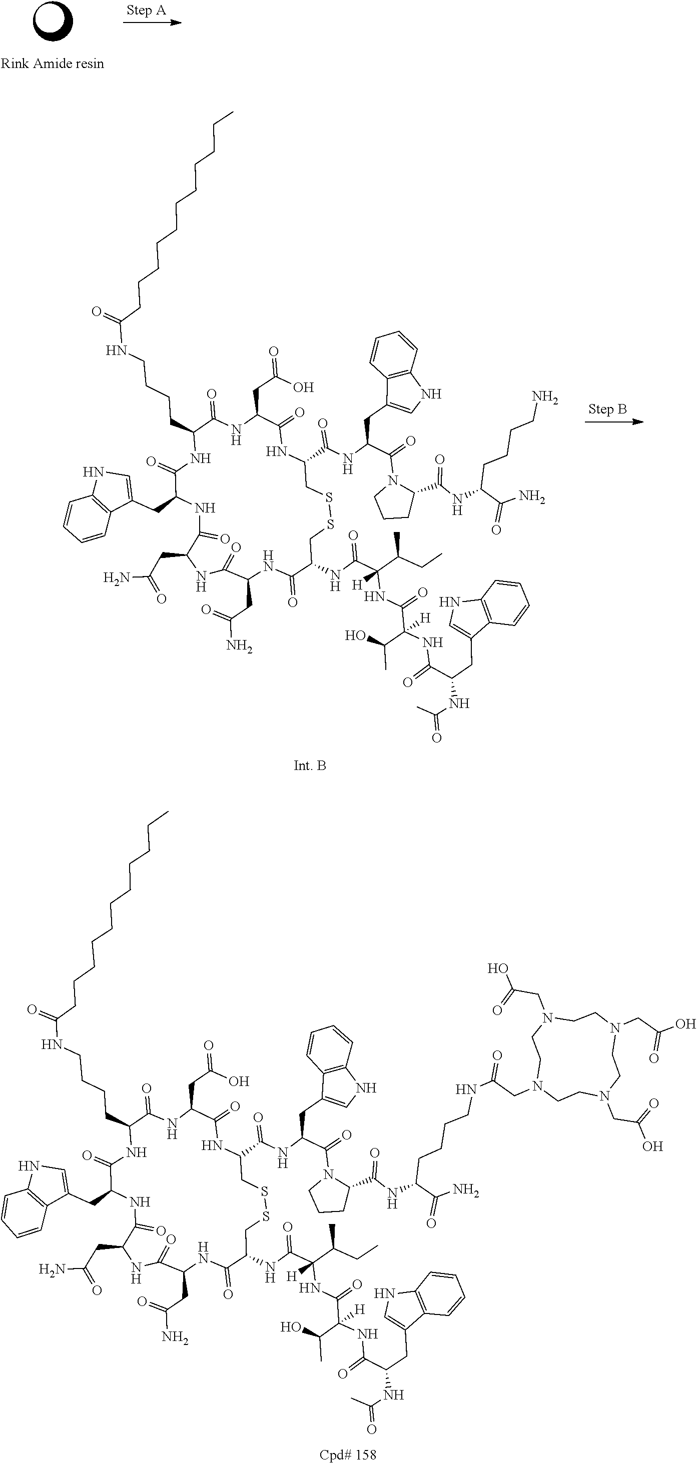

### Step A. Synthesis of Compound Int. B

The peptide was synthesized by standard Solid-phase Peptide Synthesis (SPPS) using Fmoc/t-Bu chemistry. The assembly was performed on a Rink-amide MBHA resin (100 umol, 100-200 mesh; loading 0.42 mmol/g) on the CEM Liberty Blue microwave peptide synthesizer (CEM Inc.). During peptide assembly on solid phase, the side chain protecting groups were: tert-butyl for T; trityl for C and N; Boc for W and D-Lys; Mpe for D, Dde for K.

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1M solution of DIC in DMF and Oxyma solution 1M in DMF.

Double acylation reactions were performed for T², K⁸, N⁶ and W¹¹.

N-terminal acetylation was performed for 2 minutes at 65° C. under MW irradiation with a 10% v/v solution of Ac₂O in DMF.

At the end of the assembly, a 3% hydrazine monohydrate solution in DMF (50 mL) was percolated on the resin over 15 minutes to selectively remove the Dde protecting group from the Lys.

C12 (dodecanoic acid) was incorporated manually using an equimolar solution of C12, DIC and HOBt (3 Eq, 1:1:1) in NMP at room temperature and complete acylation was monitored by ninhydrin test.

At the end of the assembly the resin was washed with DMF, DCM, Et₂O. The peptide was cleaved from solid support using 15 mL of TFA solution (v/v: 87.5% TFA, 5% H₂O, 2.5% TIPS, 5% Phenol) for approximately 1.5 hours, at room temperature. The resin was then filtered and concentrated to about 4 mL and precipitated in cold MTBE (40 mL). After centrifugation, the peptide pellets were washed with fresh cold Et₂O to remove the organic scavengers. The process was repeated twice. Final pellets were dried, and crude material was directly used for the next step.

Crude peptide was dissolved in H₂O/ACN (1 mg/mL). Iodine (saturated solution in AcOH) was added until yellow color persisted. Stirred at room temperature for 5 min, then quenched with ascorbic acid and lyophilized. Half of the material was purified by reverse-phase HPLC using preparative Waters Deltapak C4 column (200×25 mm, 300Å, 15 μm). Mobile phase A: H₂O+0.1% TFA, mobile phase B: ACN+0.1% TFA. The following gradient of eluent B was used: 30% B to 30% B over 5 min, to 45% B over 25 min, flow rate 50 mL/minute, wavelength 214 nm. Collected fractions were lyophilized to afford Compound Int. B (8.8 mg, Yield: 9.7%). LCMS anal. calc. For C92H131N21O20S2: 1915.28; found: 959.0 (M+2)²⁺

### Step B. Synthesis of Compound Cpd No. 158

Intermediate Compound B (Int. B) was dissolved in DMSO (20 mg/mL). DIPEA (10 equiv.) was added, followed by DOTA-NHS (3 equiv.). Reaction was complete after 4 h (monitored by UPLC-MS) and the mixture was quenched with TFA. Crude peptide was purified by reverse-phase HPLc using preparative Waters XBridge C4 column (150×19 mm, 300Å, 5 μm). Mobile phase A: H₂O+0.1% TFA, mobile phase B: ACN+0.1% TFA. The following gradient of eluent B was used: 35% B to 35% B over 5 min, to 55% B over 25 min, flow rate 25 mL/minute, wavelength 214 nm. Collected fractions were lyophilized to afford Cpd No. 158 (7.8 mg, Yield: 74%).

### Procedure G

### Example: Cpd No. 252

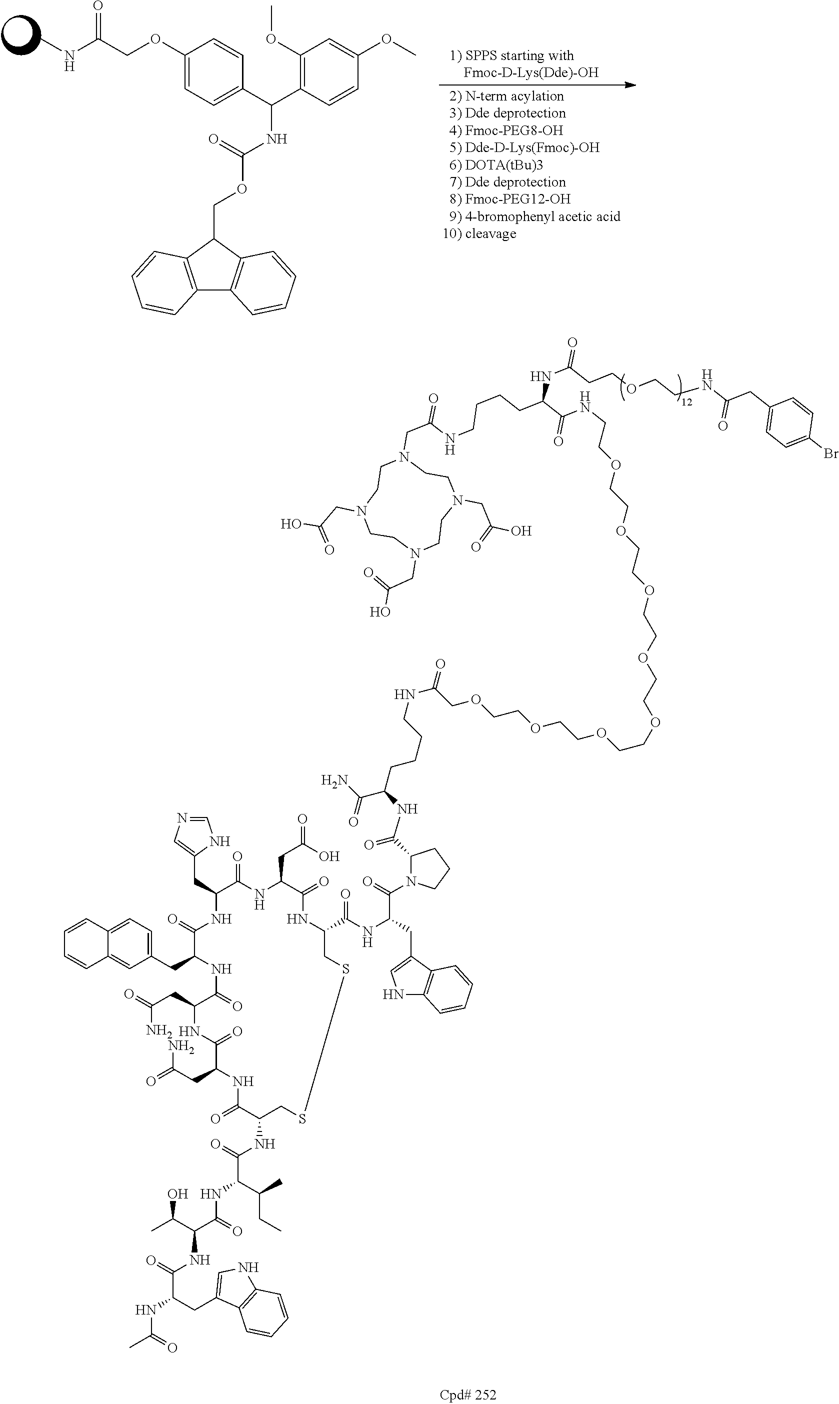

### Solid Phase Peptide Synthesis:

The peptide was synthesized by standard Solid-phase Peptide Synthesis (SPPS) using Fmoc/t-Bu chemistry. The assembly was performed on a ProTide Rink Amide resin (0.1 mmol scale, 0.19 g/mmol) on the CEM Liberty Blue microwave peptide synthesizer.

Fmoc deprotection was performed by adding 20% (v/v) piperidine in DMF (3 mL) to the resin and the mixture was heated to 90° C. for 1 minute. The resin was then washed with DMF 4 times.

Amide coupling: To the microwave reaction vessel was added Fmoc-protected amino acid (5 equiv.), DIC (10 equiv.) in DMF (1M), and OxymaPure (5 equiv.) in DMF (1M). The microwave reaction vessel was then heated to 90° C. for 4 minutes and then drained. For double couplings, this process was repeated twice.

Double acylation reactions were performed for all positions except for Fmoc-D-Lys(Dde)-OH at position 13 (single coupling).

After final Fmoc deprotection, N-terminal acetylation was performed for 2 minutes at 65° C. under MW irradiation with a 2.5 mL of 10% v/v solution of Ac₂O in DMF. The resin was then washed with 4 mL of DMF 4 times.

Orthogonal deprotection to remove Dde from D-Lys: to the microwave reaction vessel was added 4 mL of 2% hydrazine monohydrate solution in DMF, which was mixed at room temperature for 30 minutes. The resin was then washed with 4 mL of DMF 6 times.

The remaining residues were installed as following: Fmoc-PEG8-OH (double coupling), Dde-D-Lys(Fmoc)-OH (double coupling), DOTA(tBu)₃ (triple coupling), orthogonal protection to remove Dde group (2% hydrazine in DMF, 30 min), Fmoc-PEG12-OH (double coupling), and finally 4-bromophenyl acetic acid (double coupling).

At the end of the assembly the resin was washed with DMF and DCM.

### Peptide Cleavage, Cyclization and Purification:

The peptide was cleaved from the resin using 8 mL of cleavage solution (TFA/DODT/H₂O/TIS, 92.5/2.5/2.5/2.5, v/v/v/v) for 30 minutes at 40° C. using the CEM Razor peptide cleavage system. The resin was then filtered, and the peptide was precipitated with cold ether (45 mL). The precipitated peptides are then placed on to a centrifuge and allowed to spin for at least 5 minutes. The ether was decanted, leaving the peptide at the bottom of centrifuge tube. This ether precipitation process was then repeated.

The peptide was then dissolved in 15 mL of MeCN/H₂O (1/1, v/v), and 0.1 M I₂ in MeOH was added dropwise until yellow color persisted. The mixture was shaken at 20° C. for 10 minutes. The reaction was quenched by adding 0.1 M Na₂S₂O₃ dropwise until the yellow color disappeared. The reaction solution was then concentrated under reduced pressure. The crude cyclized peptide was then dissolved in 2 mL of DMSO for purification.

The crude peptide was purified by prep-HPLc using preparative Waters XBridge C18 column (100×19 mm, 5 μm OBD). Mobile phase A: H₂O+0.1% TFA, mobile phase B: ACN+0.1% TFA. The following gradient of eluent B was used: 20% B from 0-0.5 minute; 20-40% B from 0.5-14 minutes; 40-95% B from 14-15 minutes; hold at 95% B from 15-17 minutes; 20% B from 17-18 minutes. Column temperature: ambient. Flow rate: 24 mL/minute. Collected fractions were lyophilized to afford the desired product.

### General Procedure H

### Example: Cpd No. 354

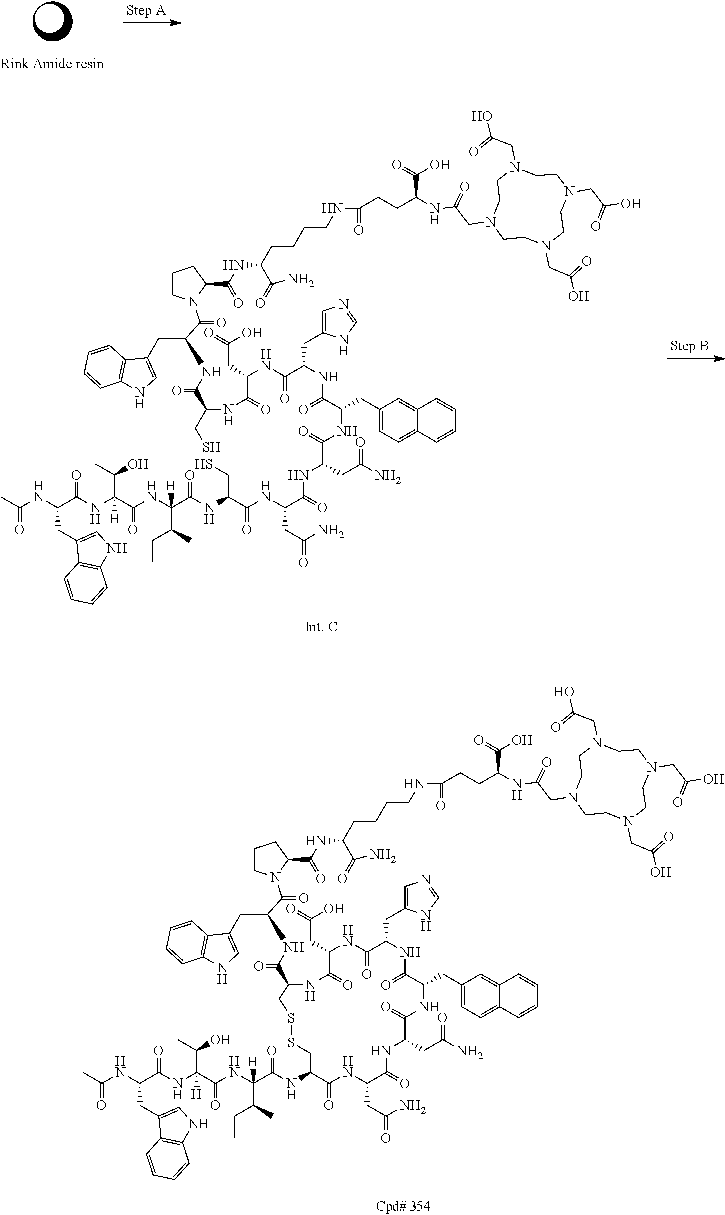

### Step A. Synthesis of Compound Int. C

The peptide was synthesized by standard Solid-phase Peptide Synthesis (SPPS) using Fmoc/t-Bu chemistry. The assembly was performed on a Rink-amide MBHA resin (100 umol, 100-200 Mesh; loading 0.42 mmol/g) on the CEM Liberty Blue microwave peptide synthesizer (CEM Inc.). During peptide assembly on solid phase, the side chain protecting groups were: tert-butyl for T and gE; trityl for C and N; Boc for W and H; Mpe for D, Dde for D-Lys.

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1M solution of DIC in DMF and Oxyma solution 1M in DMF. Double acylation reactions were performed for T², H⁸, N⁶ and W¹¹.

N-terminal acetylation was performed for 2 minutes at 65° C. under MW irradiation with a 10% v/v solution of Ac₂O in DMF.

At the end of the assembly, a 3% hydrazine monohydrate solution in DMF (50 mL) was percolated on the resin over 15 minutes to selectively remove the Dde protecting group from the Lys.

D-Lys side chain derivatization was performed manually using Fmoc-Glu-OtBu (gE) and of DOTA(tBu)₃. Acylation was performed using an equimolar solution of acid, DIC and HOBt (3 Eq, 1:1:1) in NMP at room temperature and complete acylation was monitored by ninhydrin test. Fmoc deprotection was performed using a 20% solution of piperidine in DMF, at room temperature (2×5 min).

At the end of the assembly the resin was washed with DMF, DCM, Et₂O. The peptide was cleaved from solid support using 20 mL of TFA solution (v/v: 87.5% TFA, 5% H₂O, 2.5% TIPS, 5% Phenol) for approximately 4 hours, at room temperature. The resin was then filtered and concentrated to about 4 mL and precipitated in cold MTBE (40 mL). After centrifugation, the peptide pellets were washed with fresh cold Et₂O to remove the organic scavengers. The process was repeated twice. Final pellets were dril re-suspended in H₂O and ACN 1:1 and stirred overnight. Then lyophilized to afford crude Compound Int. C (186 mg, Yield: 82%). LCMS anal. calc. For C₁₀₃H₁₄₀N₂₆O₂₉S₂: 2270.50; found: 758.5 (M+3)³′

### Step B. Synthesis of Compound Cpd No. 354

Intermediate Clound A was dissolved in H₂O/ACN (1 mg/mL). Iodine (saturated solution in AcOH) was added until yellow color persisted. Stirred at room temperature for 5 min, then quenched with ascorbic acid and lyophilized. Crude peptide was purified by reverse-phase HPLC using preparative Waters XBridge C18 column (250×50 mm, 130Å, 5 μm). Mobile phase Al₂O+0.1% TFA, mobile phase B: ACN+0.1% TFA. The following gradient of eluent B was used: 20% B to 20% B over 5 min, to 35% B over 25 min, flow rate 80 mL/minute, wavelength 214 nm. Collected fractions were lyophilized to afford 23 mg of Compound Cpd No. 354 with a purity below 90%.

Peptide was then re-purified by reverse-phase HPLC using preparative Phenomenex Luna C18 column (250×30 mm, 100Å, 5 μm). Mobile phasl: H₂O+0.1% TFA, mobile phase B: ACN+0.1% TFA. The following gradient of eluent B was used: 20% B to 20% B over 5 min, to 35% B over 25 min, flow rate 30 mL/minute, wavelength 214 nm. Collected fractions were lyophilized to afford Compound Cpd No. 354 (7 mg, Yield: 30.4%).

### Procedure L

### Example: Cpd No. 329

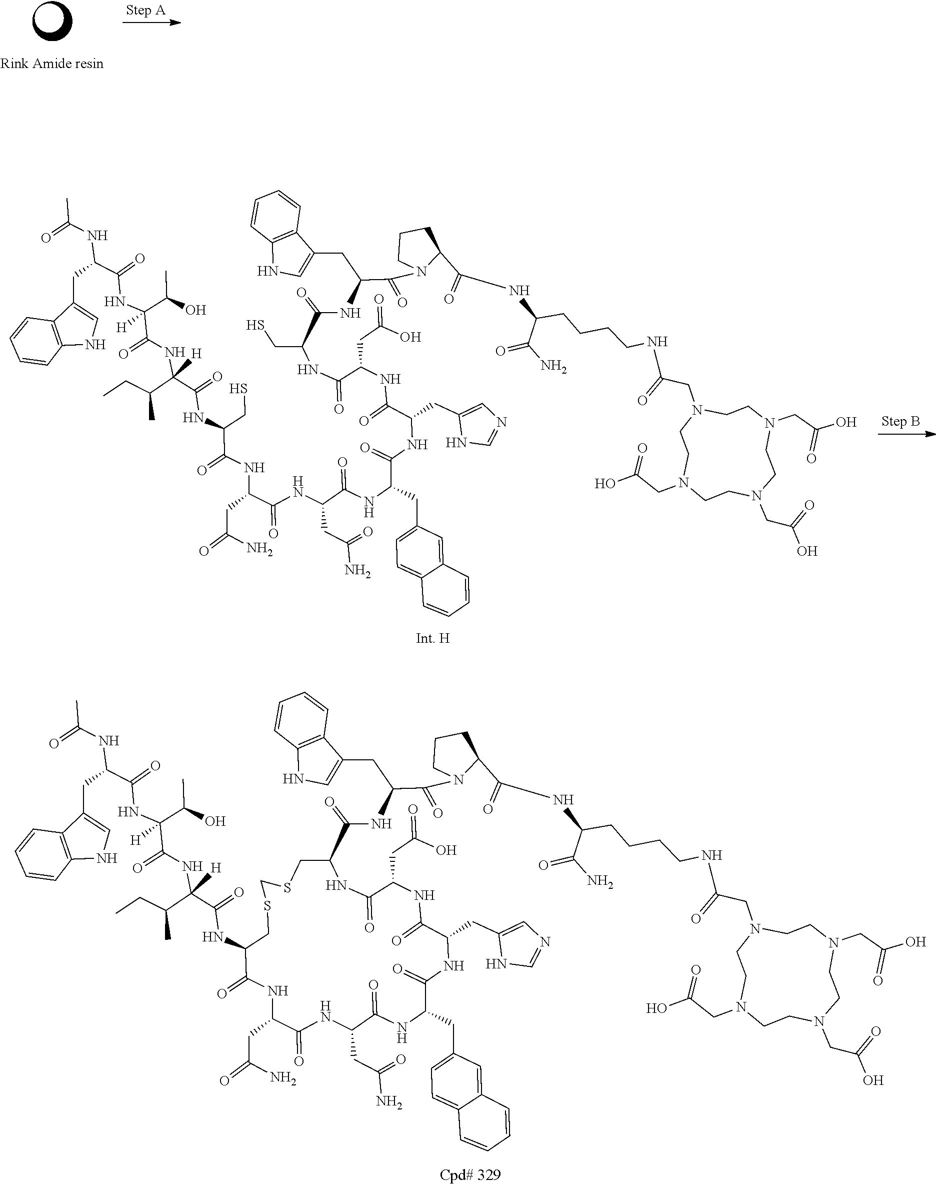

### Step A. Synthesis of Compound Int. H

The peptide was synthesized by standard Solid-phase Peptide Synthesis (SPPS) using Fmoc/t-Bu chemistry. The assembly was performed on a Rink-amide MBHA resin (250 umol, 100-200 Mesh; loading 0.42 mmol/g) on the Cem Liberty Blue microwave peptide synthesizer (CEM Inc.). During peptide assembly on solid phase, the side chain protecting groups were: tert-butyl for T; trityl for C and N; Boc for W and H; Mpe for D, Dde for L-Lys.

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1M solution of DIC in DMF and Oxyma solution 1M in DMF.

Double acylation reactions were performed for T², H⁸, and W¹¹.

N-terminal acetylation was performed for 2 minutes at 65° C. under MW irradiation with a 10% v/v solution of Ac₂O in DMF.

At the end of the assembly, a 4% hydrazine monohydrate solution in DMF (20 mL) was percolated on the resin over 15 minutes to selectively remove the Dde protecting group from the Lys.

DOTA was incorporated manually using an equimolar solution of DOTA(tBu)₃, DIC and HOBt (3 Eq, 1:1:1) in NMP at room temperature and complete acylation was monitored by ninhydrin test.

At the end of the assembly the resin was washed with DMF, DCM, Et₂O. The peptide was cleaved from solid support using 20 mL of TFA solution (v/v: 87.5% TFA, 5% H₂O, 2.5% TIPS, 5% Phenol) for approximately 4 hours, at room temperature. The resin was then filtered and concentrated to about 5 mL and precipitated in cold MTBE (45 mL). After centrifugation, the peptide pellets were washed with fresh cold Et₂O to remove the organic scavengers. The process was repeated twice. Final pellI were dried, re-suspended in H₂O and ACN 1:1 and stirred overnight. Then lyophilized to afford crude Compound Int. H. LCMS anal. calc. For C₉₈H₁₃₃N₂₅O₂₆S₂: 2141.39; found: 715.1 (M+3)^{3+.}

### Step B. Synthesis of Compound Cpd No. 329

Intermediate Compound A (210 mg) was dissolved in H₂O/ACN (1 mg/mL). TCEP HCl (3 equiv.) was added, followed by diiodomethane (50 equiv.) and DIPEA (0.5% v/v). Stirred at room temperature. Reaction was complete after 3 h (monitored by UPLC-MS). Quenched with TFA and lyophilized.

Crude peptide was purified by reverse-phase HPLC in two runs using preparative Waters XBridge C18 column (150×50 mm, 130 Å, 5 μm). Mole phase A: H₂O+0.1% TFA, mobile phase B: ACN+0.1% TFA. The following gradient of eluent B was used: 20% B to 20% B over 5 min, to 35% B over 25 min, flow rate 80 mL/minute, wavelength 214 nm. Collected fractions were lyophilized to afford the desired peptide Ia TFA salt.

Pure peptide was dissolved in H₂O/ACN (1 mg/mL) and HCl 50 mM solution (10 equiv. respect each amino group) was added. The resulting solution was lyophilized, then re-dissolved in H₂O/ACN (1 mg/mL) and lyophilized to afford Cpd No. 329.

### General Procedure N

Peptides were synthesized using the CEM Liberty Blue microwave peptide synthesizer. Standard Fmoc chemistry and couplings were used with Wang resin (0.75 mmol/g).

The Wang resin was manually loaded with the following protocol. The Wang resin was transferred to a fritted syringe and the resin was swelled by DMF. To the fritted syringe was added 4eq of Fmoc-protected amino acid, 4eq of HOBT, 4eq of DIC, and 1eq. of DMAP in a total of 5 mL of DMF. The reaction mixture was allowed to shake for 4 h. The reaction mixture was filtered and washed with DMF (4×5 mL, 30 seconds each) and DCM (4×5 mL, 30 seconds each). The resin was then placed under vacuum to dry.

Step 1. Deprotection: 20% piperidine in DMF (3 mL, 75 equiv.) was added to the resin and the mixture was heated to 90° C. for 1 minute. The resin was then washed with DMF (4×4 mL).

Step 2. Double Coupling: To the microwave reaction vessel was added Fmoc-protected amino acid (5 equiv.), DIC (10 equiv.) in DMF (1M), and OxymaPure (5 equiv.) in DMF (1M). The microwave reaction vessel is then heated to 90° C. for 4 minutes and then drained. To the reaction vessel was added Fmoc-protected amino acid (5 equiv.), DIC (10 equiv.) in DMF (1M), and OxymaPure (5 equiv.) in DMF (1M). The microwave reaction vessel is then heated to 90° C. for 4 minutes and then drained.

Step 3. Steps 1 and 2 were repeated for the remaining amino acids.

Step 4. Capping: Normal deprotection protocol used (Step 1) to remove the Fmoc group on the final amino acid. After the DMF washes (4×4 mL), 2.5 mL of 10% Ac₂O in DMF was added to the microwave reaction vessel and then heated to 65° C. for 2 minutes. The resin was then washed with DMF (4×4 mL).

Step 5. Orthogonal Deprotection: The resin is washed with DMF (2×4 mL). To the microwave reaction vessel was added 4 mL 2% Hydrazine in DMF, which was mixed at room temperature for 30 minutes. The resin is then washed with DMF (5×4 mL).

Step 6. DOTA Coupling: The final DOTA coupling is added with triple coupling conditions. To the microwave reaction vessel was added DOTA(tBu)₃ (5 equiv.), DIC (10 equiv.) in DMF (1M), and OxymaPure (5 equiv.) in DMF (1M). The microwave reaction vessel is then heated to 90° C. for 4 minutes and then drained. This step is repeated an additional 2 times.

At the end of the assembly the resin was washed with DMF and DCM.

The peptide was cleaved from the resin using 8 mL of cleavage solution (TFA/DODT/H₂O/TIS, 92.5/2.5/2.5/2.5, v/v/v/v) for 30 minutes at 40° C. using the CEM Razor peptide cleavage system. The resin was then filtered, and the peptide was precipitated with cold ether (45 mL). The precipitated peptides are then placed on to a centrifuge and allowed to spin for at least 5 minutes. The ether was decanted, leaving the peptide at the bottom of centrifuge tube. This ether precipitation process was then repeated. The peptide was then dissolved in 10 mL of MeCN/H₂O (1/1, v/v) and lyophilized to dryness.

The crude linear peptide was dissolved in 25 mL of ACN/H₂O (1:1). DODT (2 equiv., 32 uL) was added to the reaction mixture, followed by the addition of DIPEA (250 uL-400 uL) to have the reaction solution pH 8-10. Lastly, diiodomethane (20eq., 150 uL) was added to the reaction solution and the mixture was shaken at room temperature for 2 hours. The reaction was monitored by HPLC, and once the reaction was completed, the reaction was quenched with 100 μL of TFA. The reaction solvent was removed by lyophilization, and the sample was purified by HPLC.

The crude peptide was purified by prep-HPLC using preparative Waters XSelect Peptide CSH C18 column (1.9 cm i.d. x 25 cm (5 μm/130 Å)). MIle phase A: 20 mM NH₄HCO₃ in water, mobile phase B: ACN. Gradient: 20% B from 0-1 minutes; 20-50% B from 1-35 minutes; 50-90% B from 35-36 minutes; 90-95% B from 36-36.5 minutes; 95% B from 36.5-40 minutes. 95-20% B from 40-41 minutes. 20% B from 41-45 minutes. Column temp: ambient. Flow rate: 24 mL/minute. Collected fractions were lyophilized to afford the desired product.

### Procedure Q

### Example: Cpd No. 256

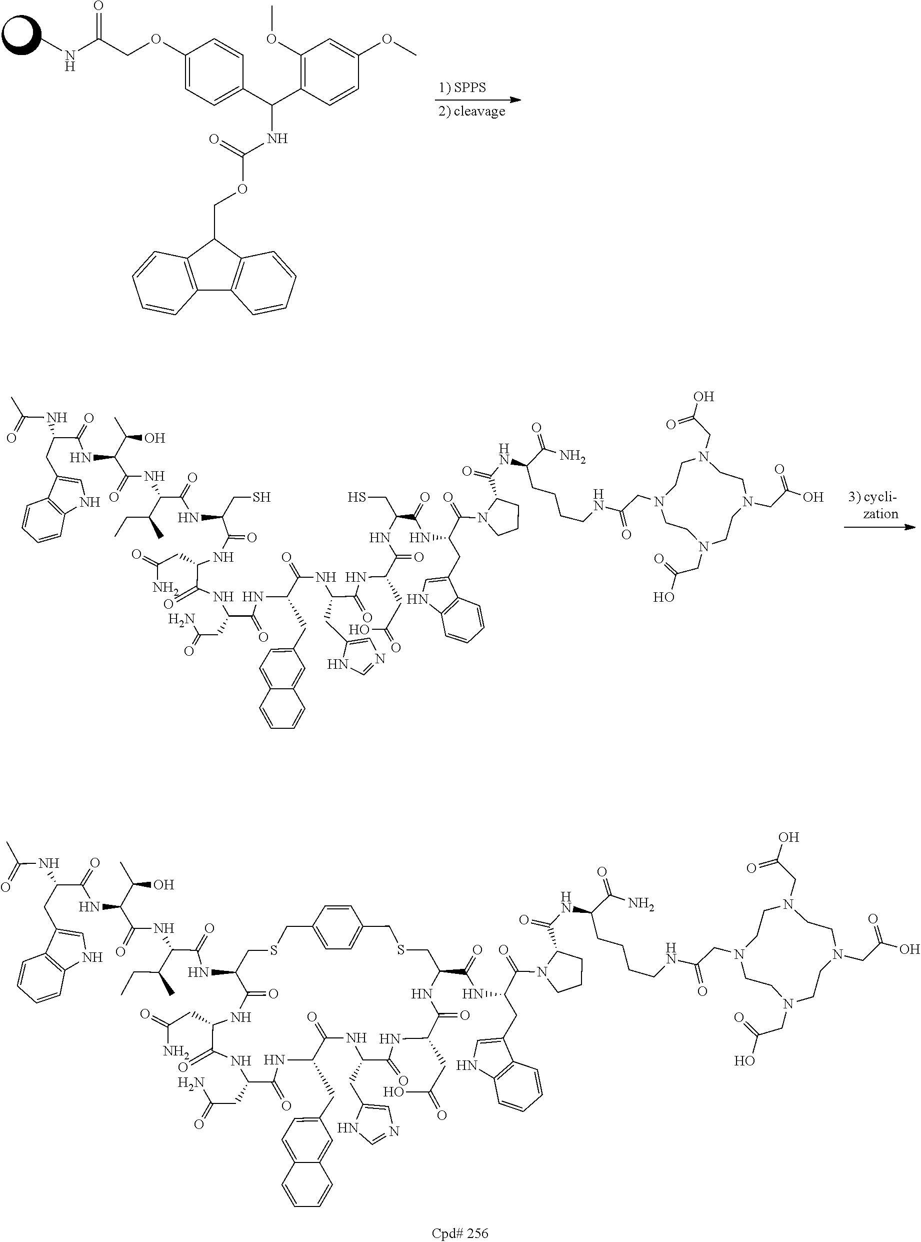

### Solid Phase Peptide Synthesis:

The peptide was synthesized by standard Solid-phase Peptide Synthesis (SPPS) using Fmoc/t-Bu chemistry. The assembly was performed on a ProTide Rink Amide resin (0.1 mmol scale, 0.19 g/mmol) on the CEM Liberty Blue microwave peptide synthesizer.

Fmoc deprotection was performed by adding 20% (v/v) piperidine in DMF (3 mL) to the resin and the mixture was heated to 90° C. for 1 minute. The resin was then washed with DMF 4 times.

Amide coupling: To the microwave reaction vessel was added Fmoc-protected amino acid (5 equiv.), DIC (10 equiv.) in DMF (1M), and OxymaPure (5 equiv.) in DMF (1M). The microwave reaction vessel was then heated to 90° C. for 4 minutes and then drained. For double couplings, this process was repeated twice.

Cycles of Fmoc deprotection and Double couplings were performed for all positions except DOTA(tBu)₃.

N-terminal acetylation was performed for 2 minutes at 65° C. under MW irradiation with a 2.5 mL of 10% v/v solution of Ac₂O in DMF. The resin was then washed with 4 mL of DMF 4 times.

Orthogonal deprotection to remove Dde from D-Lys: At the end of the assembly, to the microwave reaction vessel was added 4 mL of 2% hydrazine monohydrate solution in DMF, which was mixed at room temperature for 30 minutes. The resin was then washed with 4 mL of DMF 6 times.

DOTA was incorporated using the triple coupling conditions: To the microwave reaction vessel was added DOTA(tBu)₃ (5 equiv.), DIC (10 equiv.) in DMF (1M), and OxymaPure (5 equiv.) in DMF (1M). This process was repeated three times.

At the end of the assembly the resin was washed with DMF and DCM.

### Peptide Cleavage, Cyclization and Purification:

The peptide was cleaved from the resin using 8 mL of cleavage solution (TFA/DODT/H₂O/TIS, 92.5/2.5/2.5/2.5, v/v/v/v) for 30 minutes at 40° C. using the CEM Razor peptide cleavage system. The resin was then filtered, and the peptide was precipitated with cold ether (45 mL). The precipitated peptides are then placed on to a centrifuge and allowed to spin for at least 5 minutes. The ether was decanted, leaving the peptide at the bottom of centrifuge tube. This ether precipitation process was then repeated.

The peptide was then dissolved in 10 mL of MeCN/H₂O (1/1, v/v) and lyophilized to dryness. The resulting peptide was then dissolved in 60 mL MeCN/H₂O (1/1, v/v) and 100 μL of DIPEA was added to the mixture, 30 mg of para-DBX was added in 0.5 mL of ACN. The mixture was stirred at RT for 2 h. The reaction was quenched by adding 50 μL of TFA. The reaction solution was then concentrated under reduced pressure. The crude cyclized peptide was then dissolved in 2 mL of DMSO for purification.

The crude peptide was purified by prep-HPLC using preparative Waters XBridge C18 column (100×19 ml5 μm OBD). Mobile phase A: H₂O+0.1% TFA, mobile phase B: ACN+0.1% TFA. The following gradient of eluent B was used: 20% B from 0-0.5 minutes; 20-40% B from 0.5-14 minutes; 40-95% B from 14-15 minutes; hold at 95% B from 15-17 minutes; 20% B from 17-18 minutes. Column temperature: ambient. Flow rate: 24 mL/minute. Collected fractions were lyophilized to afford the desired product Cpd No. 256.

Table 4 below shows the synthetic procedure that was used to synthesize the compounds described herein.

TABLE 4

| Cpd | Synthetic |
|---|---|
| Nos. | procedure |
| 23 | D |
| 47 | H |
| 48 | H |
| 74 | D |
| 75 | D |
| 76 | D |
| 77 | D |
| 85 | D |
| 86 | D |
| 87 | D |
| 88 | D |
| 89 | H |
| 90 | D |
| 91 | D |
| 92 | D |
| 93 | D |
| 100 | D |
| 101 | D |
| 102 | D |
| 103 | D |
| 104 | D |
| 105 | D |
| 106 | D |
| 107 | D |
| 108 | D |
| 109 | D |
| 110 | D |
| 111 | D |
| 112 | D |
| 113 | D |
| 114 | D |
| 115 | D |
| 116 | D |
| 117 | D |
| 118 | D |
| 119 | F |
| 121 | D |
| 122 | D |
| 123 | D |
| 124 | D |
| 125 | D |
| 126 | D |
| 127 | D |
| 128 | D |
| 129 | D |
| 130 | D |
| 131 | D |
| 132 | D |
| 133 | D |
| 134 | D |
| 135 | D |
| 136 | D |
| 137 | D |
| 138 | D |
| 139 | D |
| 140 | D |
| 141 | D |
| 142 | D |
| 143 | D |
| 144 | D |
| 145 | D |
| 146 | D |
| 147 | D |
| 148 | D |
| 149 | D |
| 150 | D |
| 151 | D |
| 152 | D |
| 156 | D |
| 157 | D |
| 158 | F |
| 158 | D |
| 159 | F |
| 159 | D |
| 160 | F |
| 160 | D |
| 161 | F |
| 162 | F |
| 164 | L |
| 165 | L |
| 166 | D |
| 167 | H |
| 168 | D |
| 169 | D |
| 170 | D |
| 171 | D |
| 172 | D |
| 173 | D |
| 174 | D |
| 175 | D |
| 176 | D |
| 177 | D |
| 178 | D |
| 179 | D |
| 180 | D |
| 181 | D |
| 182 | D |
| 183 | D |
| 184 | D |
| 185 | D |
| 186 | D |
| 187 | D |
| 188 | D |
| 189 | D |
| 190 | D |
| 193 | D |
| 194 | D |
| 195 | D |
| 196 | D |
| 197 | D |
| 198 | D |
| 199 | D |
| 201 | D |
| 202 | H |
| 203 | H |
| 204 | H |
| 205 | D |
| 206 | D |
| 207 | H |
| 208 | D |
| 209 | D |
| 210 | D |
| 211 | D |
| 212 | D |
| 213 | D |
| 226 | D |
| 227 | D |
| 228 | D |
| 229 | D |
| 230 | H |
| 231 | H |
| 232 | H |
| 233 | H |
| 234 | H |
| 235 | H |
| 237 | D |
| 238 | D |
| 241 | D |
| 242 | D |
| 243 | D |
| 244 | D |
| 245 | D |
| 246 | D |
| 251 | G |
| 252 | G |
| 255 | G |
| 256 | Q |
| 257 | D |
| 258 | D |
| 259 | D |
| 260 | D |
| 261 | D |
| 263 | D |
| 264 | D |
| 265 | D |
| 266 | D |
| 267 | D |
| 268 | D |
| 269 | D |
| 270 | H |
| 271 | H |
| 272 | D |
| 273 | D |
| 274 | D |
| 275 | D |
| 276 | D |
| 277 | D |
| 278 | D |
| 279 | D |
| 280 | D |
| 281 | D |
| 282 | D |
| 283 | D |
| 284 | D |
| 285 | D |
| 286 | H |
| 287 | D |
| 288 | D |
| 289 | D |
| 290 | D |
| 291 | D |
| 292 | D |
| 293 | D |
| 294 | D |
| 295 | D |
| 296 | D |
| 297 | D |
| 298 | D |
| 299 | D |
| 326 | D |
| 329 | L |
| 330 | L |
| 344 | D |
| 345 | D |
| 346 | D |
| 347 | D |
| 348 | D |
| 349 | F |
| 352 | D |
| 353 | H |
| 354 | H |
| 355 | H |
| 356 | H |
| 357 | H |
| 358 | H |
| 359 | D |
| 360 | D |
| 361 | D |
| 362 | D |
| 363 | H |
| 364 | H |
| 365 | H |
| 366 | D |
| 367 | D |
| 368 | H |
| 369 | D |
| 371 | H |
| 372 | H |
| 373 | H |
| 374 | H |
| 375 | D |
| 376 | D |
| 409 | D |
| 410 | D |
| 411 | D |
| 412 | D |
| 413 | D |
| 414 | D |
| 415 | D |
| 426 | F |
| 427 | F |
| 451 | D |

Table 5 below shows LCMS analysis for the compounds described herein.

TABLE 5

| Cpd | Exact | Observed | |
|---|---|---|---|
| Nos. | Mass | mass | Comment |
| 23 | 2126.9 | 711.1 | [M + 3H]/3 |
| 47 | 2240 | 748 | [M + 3H]/3 |
| 48 | 2266 | 756.8 | [M + 3H]/3 |
| 74 | 2137.9 | 714.3 | [M + 3H]/3 |
| 75 | 2137.9 | 714.3 | [M + 3H]/3 |
| 76 | 2137.9 | 714.3 | [M + 3H]/3 |
| 77 | 2137.9 | 714.3 | [M + 3H]/3 |
| 85 | 2127.9 | 533.6 | [M + 4H]/4 |
| 86 | 2156.9 | 720.8 | [M + 3H]/3 |
| 87 | 2127.9 | 533.6 | [M + 4H]/4 |
| 88 | 2156.9 | 720.7 | [M + 3H]/3 |
| 89 | 2536.1 | 847.5 | [M + 3H]/3 |
| 90 | 2211 | 738.7 | [M + 3H]/3 |
| 91 | 2239 | 748.1 | [M + 3H]/3 |
| 92 | 2188 | 731.2 | [M + 3H]/3 |
| 93 | 2216 | 740.4 | [M + 3H]/3 |
| 100 | 2144.9 | 1073.8 | [M + 2H]/2 |
| 101 | 2144.9 | 1074.3 | [M + 2H]/2 |
| 102 | 2144.9 | 1073.8 | [M + 2H]/2 |
| 103 | 2140.9 | 1072.2 | [M + 2H]/2 |
| 104 | 2140.9 | 1072.2 | [M + 2H]/2 |
| 105 | 2140.9 | 1072.3 | [M + 2H]/2 |
| 106 | 2152.9 | 1077.8 | [M + 2H]/2 |
| 107 | 2210.9 | 1107.3 | [M + 2H]/2 |
| 108 | 2140.9 | 1072.1 | [M + 2H]/2 |
| 109 | 2154.9 | 1078.8 | [M + 2H]/2 |
| 110 | 2142.9 | 1072.7 | [M + 2H]/2 |
| 111 | 2142.9 | 1072.7 | [M + 2H]/2 |
| 112 | 2144.9 | 1073.7 | [M + 2H]/2 |
| 113 | 2144.9 | 1073.8 | [M + 2H]/2 |
| 114 | 2228.9 | 1116.2 | [M + 2H]/2 |
| 115 | 2174.9 | 1089.2 | [M + 2H]/2 |
| 116 | 2210.9 | 1107.1 | [M + 2H]/2 |
| 117 | 2140.9 | 1072.2 | [M + 2H]/2 |
| 118 | 2140.9 | 1072.2 | [M + 2H]/2 |
| 119 | 3051.5 | 764.8 | [M + 4H]/4 |
| 121 | 2110.9 | 705.2 | [M + 3H]/3 |
| 122 | 2124.9 | 709.6 | [M + 3H]/3 |
| 123 | 2138.9 | 714.4 | [M + 3H]/3 |
| 124 | 2110.9 | 704.9 | [M + 3H]/3 |
| 125 | 2150.9 | 718.3 | [M + 3H]/3 |
| 126 | 2122.9 | 709 | [M + 3H]/3 |
| 127 | 2136.9 | 713.9 | [M + 3H]/3 |
| 128 | 2212.8 | 739 | [M + 3H]/3 |
| 129 | 2142.9 | 715.7 | [M + 3H]/3 |
| 130 | 2156.9 | 720.3 | [M + 3H]/3 |
| 131 | 2170.9 | 724.9 | [M + 3H]/3 |
| 132 | 2142.9 | 715.6 | [M + 3H]/3 |
| 133 | 2182.9 | 728.9 | [M + 3H]/3 |
| 134 | 2154.9 | 719.7 | [M + 3H]/3 |
| 135 | 2168.9 | 724.3 | [M + 3H]/3 |
| 136 | 2244.8 | 749.6 | [M + 3H]/3 |
| 137 | 2160.9 | 1081.7 | [M + 2H]/2 |
| 138 | 2174.9 | 1088.8 | [M + 2H]/2 |
| 139 | 2188.9 | 731.1 | [M + 3H]/3 |
| 140 | 2160.9 | 721.6 | [M + 3H]/3 |
| 141 | 2200.9 | 1101.7 | [M + 2H]/2 |
| 142 | 2172.9 | 725.7 | [M + 3H]/3 |
| 143 | 2186.9 | 730.3 | [M + 3H]/3 |
| 144 | 2262.8 | 755.7 | [M + 3H]/3 |
| 145 | 2194.9 | 732.3 | [M + 3H]/3 |
| 146 | 2206.9 | 737 | [M + 3H]/3 |
| 147 | 2220.9 | 741.7 | [M + 3H]/3 |
| 148 | 2192.9 | 732.4 | [M + 3H]/3 |
| 149 | 2232.9 | 745.7 | [M + 3H]/3 |
| 150 | 2204.9 | 736.4 | [M + 3H]/3 |
| 151 | 2218.9 | 741 | [M + 3H]/3 |
| 152 | 2294.8 | 766.3 | [M + 3H]/3 |
| 156 | 2140.9 | 1072.7 | [M + 2H]/2 |
| 157 | 2130.9 | 1067.7 | [M + 2H]/2 |
| 158 | 2300.1 | 1151.8 | [M + 2H]/2 |
| 159 | 2328.1 | 1166 | [M + 2H]/2 |
| 160 | 2356.2 | 1180 | [M + 2H]/2 |
| 161 | 2295.1 | 1149.5 | [M + 2H]/2 |
| 162 | 2436.2 | 1163.5 | [M + 2H]/2 |
| 164 | 2140.9 | 715.5 | [M + 3H]/3 |
| 165 | 2151.9 | 719.4 | [M + 3H]/3 |
| 166 | 2137.9 | 1071.3 | [M + 2H]/2 |
| 167 | 2554.1 | 1279.5 | [M + 2H]/2 |
| 168 | 2142.9 | 715.6 | [M + 3H]/3 |
| 169 | 2170.9 | 725 | [M + 3H]/3 |
| 170 | 2142.9 | 715.6 | [M + 3H]/3 |
| 171 | 2182.9 | 729 | [M + 3H]/3 |
| 172 | 2154.9 | 719.7 | [M + 3H]/3 |
| 173 | 2200.9 | 735 | [M + 3H]/3 |
| 174 | 2276.9 | 760.2 | [M + 3H]/3 |
| 175 | 2168.9 | 724.3 | [M + 3H]/3 |
| 176 | 2244.9 | 749.4 | [M + 3H]/3 |
| 177 | 2174.9 | 726.5 | [M + 3H]/3 |
| 178 | 2188.9 | 730.9 | [M + 3H]/3 |
| 179 | 2202.9 | 735.5 | [M + 3H]/3 |
| 180 | 2174.9 | 726.4 | [M + 3H]/3 |
| 181 | 2214.9 | 739.5 | [M + 3H]/3 |
| 182 | 2186.9 | 730.2 | [M + 3H]/3 |
| 183 | 2142.9 | 715.6 | [M + 3H]/3 |
| 184 | 2142.9 | 715.7 | [M + 3H]/3 |
| 185 | 2154.9 | 719.6 | [M + 3H]/3 |
| 186 | 2156.9 | 720.3 | [M + 3H]/3 |
| 187 | 2168.9 | 724.6 | [M + 3H]/3 |
| 188 | 2170.9 | 725.1 | [M + 3H]/3 |
| 189 | 2182.9 | 729.1 | [M + 3H]/3 |
| 190 | 2244.9 | 1123.8 | [M + 2H]/2 |
| 193 | 2152.9 | 1078.1 | [M + 2H]/2 |
| 194 | 2121.9 | 1062.9 | [M + 2H]/2 |
| 195 | 2135.9 | 1069.9 | [M + 2H]/2 |
| 196 | 2109.9 | 1057 | [M + 2H]/2 |
| 197 | 2121.9 | 1062.9 | [M + 2H]/2 |
| 198 | 2135.9 | 1069.9 | [M + 2H]/2 |
| 199 | 2109.9 | 1056.9 | [M + 2H]/2 |
| 201 | 2208.9 | 1106.8 | [M + 2H]/2 |
| 202 | 2292 | 765.5 | [M + 3H]/3 |
| 203 | 2304 | 769.3 | [M + 3H]/3 |
| 204 | 2455.1 | 819.1 | [M + 3H]/3 |
| 205 | 2165 | 723.2 | [M + 3H]/3 |
| 206 | 2151.9 | 1077.5 | [M + 2H]/2 |
| 207 | 2539.1 | 1271.4 | [M + 2H]/2 |
| 208 | 2110.9 | 1057.3 | [M + 2H]/2 |
| 209 | 2138.9 | 714.9 | [M + 3H]/3 |
| 210 | 2128.9 | 1066 | [M + 2H]/2 |
| 211 | 2136.9 | 1070.9 | [M + 2H]/2 |
| 212 | 2101.9 | 1052.8 | [M + 2H]/2 |
| 226 | 2160.9 | 721.9 | [M + 3H]/3 |
| 227 | 2100.9 | 702.1 | [M + 3H]/3 |
| 228 | 2112.9 | 706.1 | [M + 3H]/3 |
| 229 | 2114.9 | 1059.3 | [M + 2H]/2 |
| 230 | 2554.1 | 853.3 | [M + 3H]/3 |
| 231 | 2540.1 | 1272.5 | [M + 2H]/2 |
| 232 | 2570.1 | 1287.4 | [M + 2H]/2 |
| 233 | 2267 | 1135.8 | [M + 2H]/2 |
| 234 | 2525 | 1264.8 | [M + 2H]/2 |
| 235 | 2658.2 | 1331.6 | [M + 2H]/2 |
| 237 | 2128.9 | 1066.9 | [M + 2H]/2 |
| 238 | 2172 | 1087.7 | [M + 2H]/2 |
| 241 | 2156.9 | 720.2 | [M + 3H]/3 |
| 242 | 2188.9 | 730.8 | [M + 3H]/3 |
| 243 | 2202.9 | 735.4 | [M + 3H]/3 |
| 244 | 2174.9 | 1085.5 | [M + 2H]/2 |
| 245 | 2214.9 | 739.7 | [M + 3H]/3 |
| 246 | 2276.9 | 760.1 | [M + 3H]/3 |
| 251 | 3280.4 | 822.3 | [M + 4H]/4 |
| 252 | 3470.5 | 869.7 | [M + 4H]/4 |
| 255 | 3998.9 | 1002 | [M + 4H]/4 |
| 256 | 2242 | 1122.9 | [M + 2H]/2 |
| 257 | 2174.9 | 726.2 | [M + 3H]/3 |
| 258 | 2200.9 | 734.8 | [M + 3H]/3 |
| 259 | 2152 | 718.4 | [M + 3H]/3 |
| 260 | 2178 | 727.3 | [M + 3H]/3 |
| 261 | 2180 | 727.9 | [M + 3H]/3 |
| 262 | 2138.9 | 714.2 | [M + 3H]/3 |
| 263 | 2171.9 | 725.3 | [M + 3H]/3 |
| 264 | 2171.9 | 725.4 | [M + 3H]/3 |
| 265 | 2171.9 | 725.3 | [M + 3H]/3 |
| 266 | 2171.9 | 725.3 | [M + 3H]/3 |
| 267 | 2178 | 727.3 | [M + 3H]/3 |
| 268 | 2180 | 1091.2 | [M + 2H]/2 |
| 269 | 2186.9 | 730.2 | [M + 3H]/3 |
| 270 | 2385.1 | 796.3 | [M + 3H]/3 |
| 271 | 2385.1 | 796.3 | [M + 3H]/3 |
| 272 | 2160.9 | 1082.2 | [M + 2H]/2 |
| 273 | 2133.9 | 1068.5 | [M + 2H]/2 |
| 274 | 2118.9 | 1061 | [M + 2H]/2 |
| 275 | 2148.9 | 1075.9 | [M + 2H]/2 |
| 276 | 2133.9 | 1068.8 | [M + 2H]/2 |
| 277 | 2141.9 | 1072.7 | [M + 2H]/2 |
| 278 | 2156.9 | 1080.1 | [M + 2H]/2 |
| 279 | 2141.9 | 1072.7 | [M + 2H]/2 |
| 280 | 2204 | 1103.7 | [M + 2H]/2 |
| 281 | 2178 | 1090.7 | [M + 2H]/2 |
| 282 | 2192 | 1097.8 | [M + 2H]/2 |
| 283 | 2203 | 1103.2 | [M + 2H]/2 |
| 284 | 2160 | 721.3 | [M + 3H]/3 |
| 285 | 2190 | 1096.7 | [M + 2H]/2 |
| 286 | 2343.1 | 1173.3 | [M + 2H]/2 |
| 287 | 2198 | 1100.6 | [M + 2H]/2 |
| 288 | 2171.9 | 1087.6 | [M + 2H]/2 |
| 289 | 2186 | 1094.8 | [M + 2H]/2 |
| 290 | 2198 | 1100.8 | [M + 2H]/2 |
| 291 | 2171.9 | 1087.8 | [M + 2H]/2 |
| 292 | 2142.9 | 715.7 | [M + 3H]/3 |
| 293 | 2142.9 | 715.8 | [M + 3H]/3 |
| 294 | 2140.9 | 715 | [M + 3H]/3 |
| 295 | 2140.9 | 715 | [M + 3H]/3 |
| 296 | 2156.9 | 720.3 | [M + 3H]/3 |
| 297 | 2160.9 | 721.7 | [M + 3H]/3 |
| 298 | 2140.9 | 714.8 | [M + 3H]/3 |
| 299 | 2156.9 | 720.3 | [M + 3H]/3 |
| 326 | 2351.2 | 785.2 | [M + 3H]/3 |
| 329 | 2151.9 | 1077.5 | [M + 2H]/2 |
| 330 | 2165.9 | 723.7 | [M + 3H]/3 |
| 344 | 2167.9 | 724.5 | [M + 3H]/3 |
| 345 | 2157 | 721.1 | [M + 3H]/3 |
| 346 | 2324.1 | 776.5 | [M + 3H]/3 |
| 347 | 2352.1 | 785.6 | [M + 3H]/3 |
| 348 | 2380.1 | 795.2 | [M + 3H]/3 |
| 349 | 2297 | 1150.7 | [M + 2H]/2 |
| 352 | 2054.9 | 687.1 | [M + 3H]/3 |
| 353 | 2570.1 | 859.1 | [M + 3H]/3 |
| 354 | 2267 | 757.7 | [M + 3H]/3 |
| 355 | 2525 | 843.6 | [M + 3H]/3 |
| 356 | 2658.2 | 1331.9 | [M + 2H]/2 |
| 357 | 2853.3 | 1428.8 | [M + 2H]/2 |
| 358 | 2852.3 | 1428.3 | [M + 2H]/2 |
| 359 | 2137.9 | 714.4 | [M + 3H]/3 |
| 360 | 2137.9 | 714.7 | [M + 3H]/3 |
| 361 | 2177.9 | 728.1 | [M + 3H]/3 |
| 362 | 2151.9 | 719.2 | [M + 3H]/3 |
| 363 | 2690.3 | 1346.8 | [M + 2H]/2 |
| 364 | 2690.3 | 897.8 | [M + 3H]/3 |
| 365 | 2540.1 | 848.8 | [M + 3H]/3 |
| 366 | 2148.9 | 718.3 | [M + 3H]/3 |
| 367 | 2148.9 | 718.2 | [M + 3H]/3 |
| 368 | 2698.2 | 901.2 | [M + 3H]/3 |
| 369 | 2327.1 | 1164.2 | [M + 2H]/2 |
| 371 | 2493.1 | 832.6 | [M + 3H]/3 |
| 372 | 2366.1 | 1184.1 | [M + 2H]/2 |
| 373 | 2416.1 | 806.9 | [M + 3H]/3 |
| 374 | 2271 | 1137.1 | [M + 2H]/2 |
| 375 | 2157 | 1080.6 | [M + 2H]/2 |
| 376 | 2115.9 | 1059.3 | [M + 2H]/2 |
| 409 | 2140.9 | 1071.9 | [M + 2H]/2 |
| 410 | 2140.9 | 714.8 | [M + 3H]/3 |
| 411 | 2049.9 | 1026.3 | [M + 2H]/2 |
| 412 | 2022.9 | 1012.3 | [M + 2H]/2 |
| 413 | 2049.9 | 1026.3 | [M + 2H]/2 |
| 414 | 2099.9 | 701.3 | [M + 3H]/3 |
| 415 | 2072.9 | 1037.4 | [M + 2H]/2 |
| 426 | 3690 | 1846.9 | [M + 2H]/2 |
| 427 | 3678 | 1840.8 | [M + 2H]/2 |
| 451 | 2152.0 | 718.4 | [M + 3H]/3 |

### Example 3. Binding Affinity of Targeting Moieties Peptides to DLL3

### Affinity Determination by Surface Plasmon Resonance (SPR)

SPR studies were performed on the compounds disclosed herein using the following protocol.

Procedure: The binding affinity and binding kinetic parameters at 25° C. were determined using Biacore S200 or biacore 8K instrument. Biotinylated target ligand human DLL3 was immobilized on a streptavidin sensor chip. A reference surface was set up for nonspecific binding and refractive index changes. For analysis of the kinetics of interactions, varying concentrations of samples were injected at a flow rate of 100 μL/minute using running buffer containing 20 mM phosphate buffer with 2.7 mM KCl, 137 mM NaCl, 0.05% Surfactant P20 and 2% DMSO. Sensorgram curves were fitted with 1:1 binding model, and affinity and kinetic parameters were obtained using Biacore Insight evaluation 4.0. software.

Table 6 below shows the K_{D} values obtained by the SPR assay for a group of selected compounds. In this Table, “A” represents K_{D}≤1.0 nM; “B” represents 1.0 nM<K_{D}≤10 nM; “C” represents 10 nM<K_{D}≤100 nM; “D” represents 100 nM<K_{D}≤300 nM.

TABLE 6

| Cpd Nos. | SPR K_{D} | Cpd Nos. | SPR K_{D} | Cpd Nos. | SPR K_{D} |
|---|---|---|---|---|---|
| 23 | B | 138 | C | 233 | A |
| 47 | B | 139 | B | 234 | A |
| 48 | B | 140 | C | 235 | A |
| 74 | B | 141 | B | 237 | A |
| 75 | B | 142 | B | 238 | B |
| 76 | B | 143 | B | 241 | B |
| 77 | C | 144 | B | 242 | A |
| 85 | B | 145 | B | 243 | A |
| 86 | C | 146 | A | 244 | A |
| 87 | C | 147 | B | 245 | A |
| 88 | D | 148 | B | 246 | A |
| 89 | B | 149 | B | 251 | A |
| 90 | C | 150 | B | 252 | A |
| 91 | C | 151 | B | 255 | B |
| 92 | C | 152 | B | 256 | B |
| 93 | C | 164 | B | 257 | A |
| 100 | A | 165 | A | 258 | A |
| 105 | B | 166 | A | 259 | A |
| 106 | A | 167 | A | 260 | A |
| 107 | C | 193 | A | 261 | A |
| 113 | A | 194 | A | 262 | A |
| 114 | A | 195 | A | 263 | A |
| 115 | B | 196 | A | 264 | A |
| 116 | C | 197 | A | 265 | A |
| 118 | C | 198 | A | 266 | A |
| 121 | C | 199 | A | 267 | A |
| 122 | B | 202 | B | 268 | B |
| 123 | C | 203 | A | 269 | A |
| 124 | C | 204 | A | 270 | A |
| 125 | B | 206 | A | 271 | A |
| 126 | C | 207 | A | 273 | B |
| 127 | C | 208 | A | 274 | B |
| 128 | B | 209 | A | 275 | B |
| 129 | C | 211 | A | 276 | C |
| 130 | B | 213 | A | 277 | A |
| 131 | B | 226 | B | 278 | B |
| 132 | B | 227 | B | 279 | B |
| 133 | B | 228 | B | 280 | A |
| 134 | B | 229 | C | 281 | A |
| 135 | B | 230 | A | 282 | A |
| 136 | B | 231 | A | 283 | A |
| 137 | B | 232 | A | 284 | A |

| Cpd Nos. | SPR K_{D} | Cpd Nos. | SPR K_{D} |
|---|---|---|---|
| 285 | A | 230* | A |
| 286 | A | 231* | A |
| 287 | A | 234* | A |
| 288 | A | 367* | A |
| 289 | A | 409 | C |
| 290 | A | 410 | B |
| 295 | A | 426 | B |
| 296 | A | 427 | C |
| 298 | A | 451 | A |
| 299 | A | | |
| 326 | B | | |
| 329 | A | | |
| 330 | A | | |
| 344 | A | | |
| 345 | B | | |
| 346 | C | | |
| 348 | D | | |
| 349 | B | | |
| 352 | A | | |
| 353 | A | | |
| 354 | A | | |
| 355 | A | | |
| 356 | A | | |
| 357 | A | | |
| 358 | A | | |
| 359 | A | | |
| 360 | A | | |
| 362 | A | | |
| 363 | A | | |
| 364 | A | | |
| 365 | A | | |
| 366 | A | | |
| 367 | A | | |
| 368 | B | | |
| 369 | B | | |
| 165* | A | | |
| 371 | A | | |
| 372 | A | | |
| 373 | A | | |
| 374 | A | | |
| 376 | A | | |
| 166* | A | | |

*[^{nat}Lu]Lu -labelled peptides.

### Example 4. Cell Binding, Internalization, and Radiolabeling of Targeting Moieties Peptides General Procedure for Radiolabeling of Chelator-Peptides RPTs with [¹⁷⁷Lu]LuCl₃

[¹⁷⁷Lu]LuCl₃ was received from venders in HCl solution. For every mCi of [¹⁷⁷Lu]LuCl₃ added to the reaction vial (1.5 mL Eppendorf vial), ammonium acetate buffer (0.2 M, pH 4.9, 100 μL, containing 1% w/v ascorbic acid and 6% v/v ethanol) and peptide conjugate (1 nmol). A peptide conjugate of the present disclosure is also referred to herein as a “chelator-peptide”. The pH of the solution was determined to be approximately 5 by using pH strips. The reaction vial was incubated at 80° C., 700 RPM for 17-20 minutes. A sample from the reaction was analyzed by RP-HPLC using an Agilent Infinity II 1260 HPLC to determine reaction completion and radiochemical purity of [¹⁷⁷Lu]Lu-chelator-peptide. HPLC conditions: Waters XBridge BEH C18 Column, 130 Å, 3.5 um, 4.6 mm×250 mm; mobile phase: Solvent A=water (with 0.1% formic acid), solvent B=acetonitrile (with 0.1% formic acid). Gradients: 25-45% B in 10 minutes, 45-65% B in 12 minutes, 65-90% in 6 minutes at a flow rate of 0.5 mL/minute. Required amounts of the product was formulated in 1% PBSA for cell studies.

General Procedure for Radiolabeling of Chelator-Peptides with [²²⁵Ac]Ac

A peptide conjugate (also referred to herein as “chelator-peptide”) of the present disclosure is radiolabeled with [²²⁵Ac]Ac isotope in a reaction comprising an acetate or equivalent buffer with excipients and ethanol. The reaction mixture is heated to achieve radiolabeling, for example, the reaction mixture is heated at 90° C. for 15 minutes. Radiolabeled [²²⁵Ac]Ac-chelator-peptide is further diluted to the desired radioactive concentration with a formulation buffer comprising additional excipients. A sample of the radiolabeled [²²⁵Ac]Ac-chelator-peptide product is spiked with DTPA, analyzed by RP-HPLC, and fractions are collected every 12 seconds. After secular equilibrium is achieved between [²²⁵Ac]Ac and its daughter isotopes (>6 hr post-collection), the fractions are analyzed using gamma spectroscopy, and the resulting CPM are plotted as a function of time. The radiochemical purity (% RCP) data are obtained from this reconstructed chromatography.

### Reagents and Materials:

| Reagent/Material | Manufacturer | Category Number |
|---|---|---|
| RPMI 1640 Medium | Gibco | 11875093 |
| DMEM (1X) | Gibco | 11965-084 |
| Fetal Bovine Serum | R&D Systems | S11195 |
| TrypLE Select Enzyme (1x) | Gibco | 12563029 |
| Versene (1X) | Thermo Fisher | 15040-066 |
| PBS pH 7.2 | Gibco | 10010001 |
| Bovine Serum Albumin | Sigma Aldrich | A9576 |
| Distilled Water | Gibco | 15230162 |
| PBS + 1% Bovine Serum Albumin | In House | NA |
| Sodium Hydroxide (NaOH) | Thermo Fisher | A4782902 |
| Glycine | Thermo Fisher | J61855-AP |
| Acid Wash (Glycine + Water) | In House | NA |
| 10% NaOH (NaOH + Water) | In House | NA |
| Trypan Blue | Gibco | 15250061 |

### Instruments:

| Instrument | Manufacturer | Category Number |
|---|---|---|
| Biosafety Cabinet | Thermo Scientific | 1389-M |
| Incubator | Thermo Scientific | 381 |
| Gamma Counter | Perkin Elmer | 2470 |
| Dry Block Heating Shaker | Eppendorf | |
| Countess Cell Counter | Thermo Fisher | A49893 |
| Countess Cell Counting Chamber | Thermo Fisher | C10283 |
| Slides | | |
| Tissue Culture Treated Flask 175 cm2 | Falcon | 353112 |
| Bio Lite 75 cm2 Flask | Thermo Fisher | 130190 |

### Method:

### Cell Preparation

The study was conducted using SHP-77, CT26.WT, and CT26.DLL3 cells (Table 7) Adherent cell studies: The cells were cultured in appropriate culture media (20 mL) in tissue culture treated T150 flasks at 37° C. and 5% CO2. Adherent cells were detached (60-70% confluent) using 5 mL Versene at 37° C. for 3 minutes. After confirming the viability using countess and count of detached cells using Trypan blue, the cells were centrifuged at 4° C. for 5 minutes (1,000 rpm). The cell pellet obtained was washed once with PBS and resuspended in 1% PBSA to obtain the desired cell concentration (5-25 million cells/mL).

Suspension cell studies: The cells were cultured in appropriate culture media (20 mL) in tissue culture treated T150 flasks at 37° C. and 5% CO2. After confirming the viability and count of detached cells using Trypan blue, the cells were centrifuged at 4° C. for 5 minutes (1,000 rpm). The cell pellet obtained was washed once with PBS and resuspended in 1% PBSA to obtain the desired cell concentration (5-25 million cells/mL).

Cell numbers used for the assay were in the range of 5 million cells/condition −25 million cells/condition.

TABLE 7
Cell line information

| | | | | Growth | |
|---|---|---|---|---|---|
| Name | Source | Catalogue # | Growth mode | Conditions | Growth Media |
| SHP-77 | ATCC | CRL-2195 | Suspension/ | 37° C.; 5% | RPMI 1640 + 10% |
| | | | Adherent | CO₂ | FBS |
| CT-26. WT | ATCC | CRL-2638 | Adherent | 37° C.; 5% | DMEM (High |
| | | | | CO₂ | Glucose) + 10% |
| | | | | | FBS |
| CT-26.DLL3 | Creative | CSC- | Adherent | 37° C.; 5% | DMEM (High |
| | Biogene | R00602 | | CO₂ | Glucose) + 10% |
| | | | | | FBS |

### Study Execution

Total uptake: Cells were incubated with 300 μL (0.8 μCi) of the incubation buffer (containing radiolabeled peptide [¹⁷⁷Lu] Lu-test peptide) for 1 hour at 37° C. (Table 8). Post incubation, the cells were pelleted and washed 2 times using cold PBS+1% BSA. The supernatant and washes were combined and counted together as “unbound fraction” in a gamma counter. The cells were then resuspended in 300 μL of 1% PBS+1% BSA and counted in the gamma counter as “bound fraction”.

Internalization: The resuspended cells in 300 μl of 1% PBSA and counted in the gamma counter as “bound fraction” were collected in 1.5 mL Eppendorf tube and pelleted. The pelleted cells were incubated with 300 μl of 50 mM Glycine pH=2.5 for 3 minutes and then pelleted discarding the supernatant. This process was repeated one more time. The pelleted cells were washed with 300 μl of cold 1% PBSA and then resuspended in 300 μl 0.3M NaOH. This was counted in the gamma counter as “internalized fraction”.

TABLE 8
Summary of the experimental condition

| | Adherent/ | | | | | | |
|---|---|---|---|---|---|---|---|
| Cell | Suspension/ | Dissociation | Cell no. | Binding | Incubation | Incubation | Thermomixer |
| Name | Mixed | Method | (Million, M) | Media | Time | Temp | Speed |
| SHP-77 | Mixed | Versene | 5-25M | 1% PBSA | 1 hour | 37° C. | 700 rpm |
| CT26.WT | Adherent | Versene | 5-25M | 1% PBSA | 1 hour | 37° C. | 700 rpm |
| CT26.DLL3 | Adherent | Versene | 5-25M | 1% PBSA | 1 hour | 37° C. | 700 rpm |

SHP-77 cells are human-derived, non-engineered SCLC cells.
CT26.WT cells do not express DLL3 and used as a negative control.
CT26.DLL3 are engineered cells to express DLL3.

Tables 9 and 10 below shows the cell assay data for a group of selected compounds. In these Tables, “A” represents %≤25; “B” represents 25<%≤50; “C” represents %>50.

TABLE 9
Cell Data (CT26 DLL3)

| Cpd | | | % Internalization |
|---|---|---|---|
| Nos. | Cell line | % Binding | (out of bound fraction) |
| 75 | CT26 DLL3 | B | B |
| 263 | CT26 DLL3 | A | B |
| 264 | CT26 DLL3 | A | B |
| 23 | CT26 DLL3 | A | N/A |
| 326 | CT26 DLL3 | A | C |
| 211 | CT26 DLL3 | A | B |
| 245 | CT26 DLL3 | A | B |
| 259 | CT26 DLL3 | B | N/A |
| 260 | CT26 DLL3 | B | N/A |
| 237 | CT26 DLL3 | A | N/A |
| 261 | CT26 DLL3 | A | B |
| 266 | CT26 DLL3 | A | A |
| 267 | CT26 DLL3 | A | B |

TABLE 10
Cell Data (SHP77)

| | | | % Internalization |
|---|---|---|---|
| Cpd Nos. | Cell line | % Binding | (out of bound fraction) |
| 146 | SHP77 | A | N/A |
| 75 | SHP77 | A | N/A |
| 165 | SHP77 | A | N/A |
| 23 | SHP77 | A | N/A |
| 326 | SHP77 | A | B |
| 166 | SHP77 | A | N/A |

### Example 5. Biodistribution

### General Protocols for Ex Vivo Biodistribution Studies

Mice were intravenously injected (via the lateral tail vein) with a bolus dose of the radiolabeled peptides for ex vivo biodistribution studies when tumor volumes were in range of 200-400 mm³ for xenograft mice and when age of the mice was 5-7 weeks for tumor naïve mice studies. To assess the amount of radionuclide injected into individual mice the syringe was assayed in a dose calibrator before and after injection for [¹⁷⁷Lu]Lu-peptide.

Mice were euthanized at pre-determined time points and selected tissues were resected and collected into pre-weighed tubes. The tubes were then reweighed post resection, the difference giving the weight of each tissue. Radioactivity in each tissue was measured using a gamma counter. Counts were decay corrected to the time of injection and percentage of injected activity per gram (% IA/g) was calculated for each tissue based on the injected activity into each individual mouse. Injected activity was converted to counts based on the sensitivity of the gamma counter. The counts in tissue were then converted to the percentage of injected activity and this was divided by the mass of tissue to give % IA/g.

TABLE 11
Radioactivity concentration (% IA/g) of tested compounds in different
organs in nude mice bearing SHP-77 tumors (n = 3) at 24 hours
post-injection. “A” represents 0 < % IA/g ≤ 5 in tumor; “B”
represents % IA/g > 5 in tumor; “C” represents 0 < % IA/g ≤ 10 in
kidney; “D” represents % IA/g > 10 in kidney.

| | | tumor uptake | kidney uptake |
|---|---|---|---|
| Cpd No. | Labelling | (% IA/g) | (% IA/g) |
| 23 | [¹⁷⁷Lu]Lu | A | D |
| 47 | [¹⁷⁷Lu]Lu | A | D |
| 75 | [¹⁷⁷Lu]Lu | A | D |
| 146 | [¹⁷⁷Lu]Lu | A | C |
| 211 | [¹⁷⁷Lu]Lu | A | D |
| 245 | [¹⁷⁷Lu]Lu | A | D |
| 252 | [¹⁷⁷Lu]Lu | A | D |
| 260 | [¹⁷⁷Lu]Lu | A | C |
| 261 | [¹⁷⁷Lu]Lu | A | C |
| 263 | [¹⁷⁷Lu]Lu | A | C |
| 264 | [¹⁷⁷Lu]Lu | A | C |
| 265 | [¹⁷⁷Lu]Lu | A | D |
| 266 | [¹⁷⁷Lu]Lu | A | C |
| 267 | [¹⁷⁷Lu]Lu | A | C |
| 280 | [¹⁷⁷Lu]Lu | A | C |
| 326 | [¹⁷⁷Lu]Lu | A | C |
| 451 | [¹⁷⁷Lu]Lu | A | D |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A cyclic peptide of Formula B: 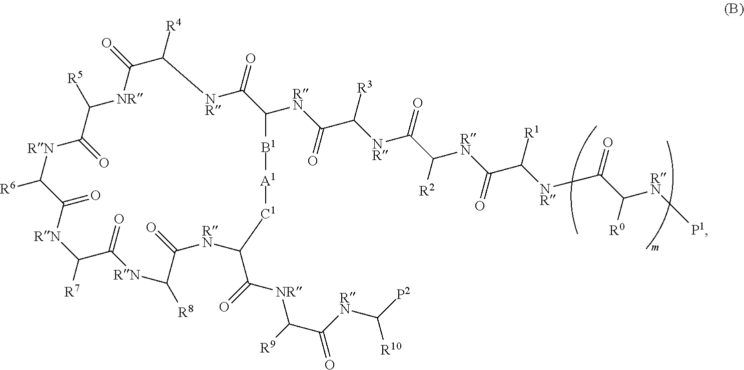
or a pharmaceutically acceptable salt thereof,
wherein:
P¹ is selected from: H, 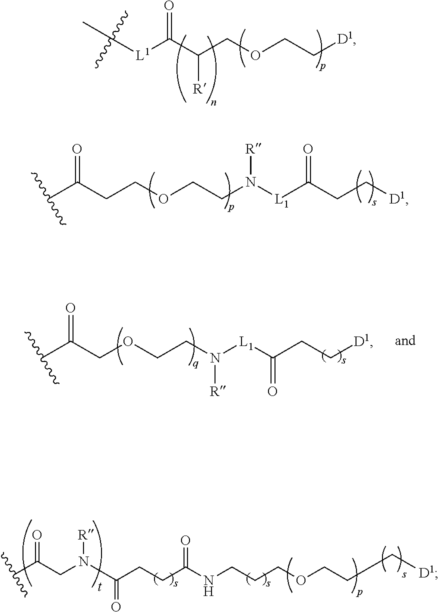
D¹ is selected from H, CH₃, and C(O)OH;
L¹ is absent or selected from 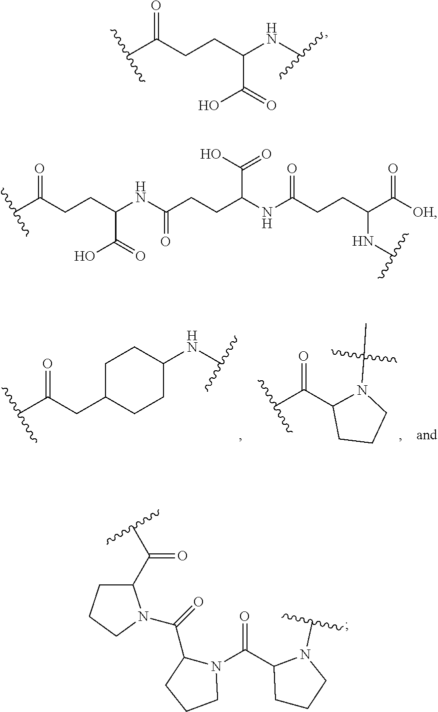 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]
wherein the amino group of L¹ connects to the carbonyl group of P1 to form an amide bond;
P² is -L²-Chelator or 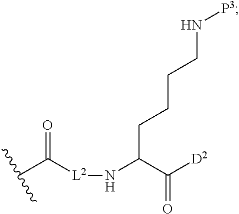
D² is OH or NH₂;
L² is absent or selected from: 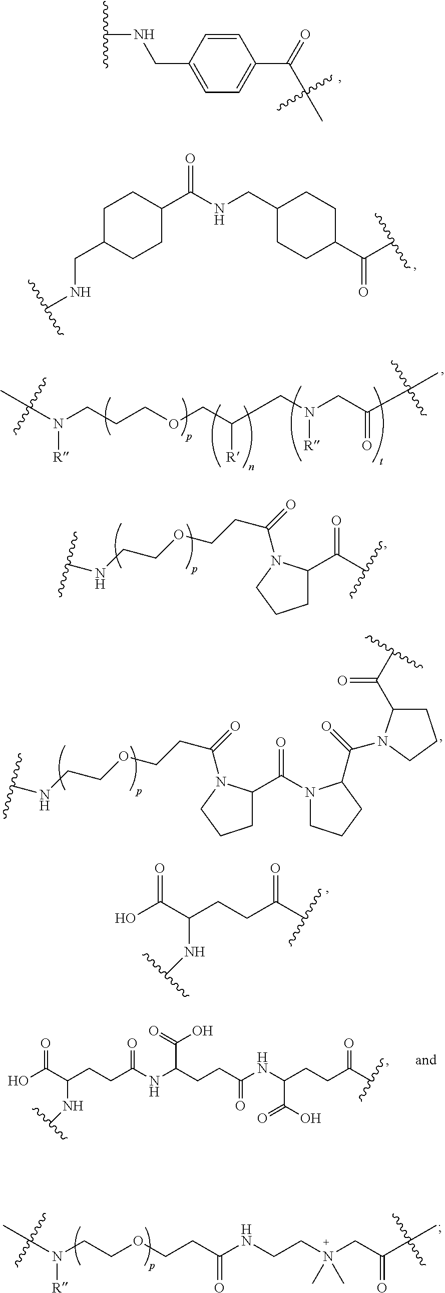
wherein the amino group of L² connects to the carbonyl group of P² or Chelator to form an amide bond;
P³ is selected from -L³-Chelator, 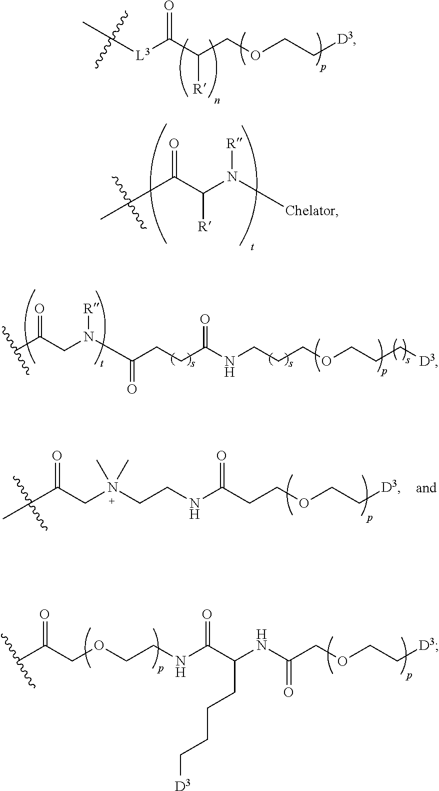
L³ is absent or independently selected from 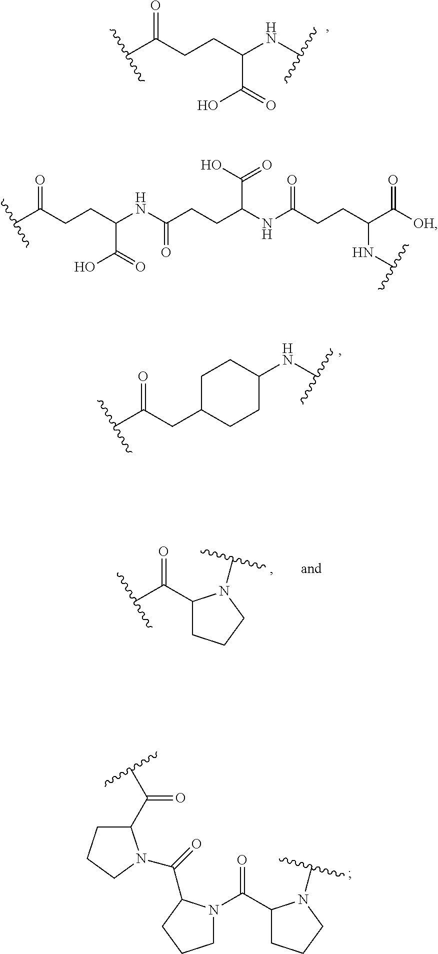 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]
wherein the carbonyl group of L³ connects to an amine group of P² to form an amide bond;
D³ is —NR″-Chelator;
R⁰ is an amino acid side chain of a natural amino acid or an amino acid side chain of an unnatural amino acid;
R¹ is an amino acid side chain of a natural amino acid or an amino acid side chain of an unnatural amino acid;
R² is an amino acid side chain of a natural amino acid or an amino acid side chain of an unnatural amino acid;
R³ is an amino acid side chain of a natural amino acid or an amino acid side chain of an unnatural amino acid;
B¹ is C₁₋₆ alkylene;
C¹ is C₁₋₆ alkylene;
A¹ is selected from: 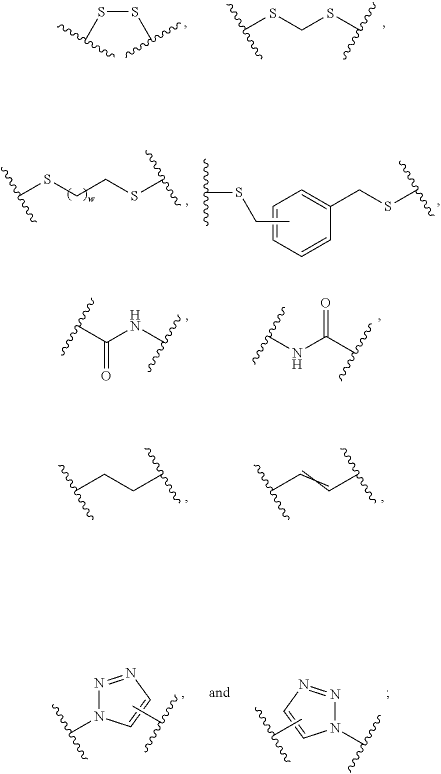
wherein w is selected from 1, 2, or 3;
R⁴ is an amino acid side chain of a natural amino acid or an amino acid side chain of an unnatural amino acid, both of which are optionally substituted with CH₂C(O)OH or C(O)(CH₂CH₂O)ₚ(CH₂)₂N(CH₃)₃⁺;
R⁵ is an amino acid side chain of a natural amino acid or an amino acid side chain of an unnatural amino acid;
R⁶ is an amino acid side chain of a natural amino acid or an amino acid side chain of an unnatural amino acid;
R⁷ is selected from:
(i) an amino acid side chain of a natural amino acid,
(ii) an amino acid side chain of an unnatural amino acid, or
(iii) selected from the group consisting of: 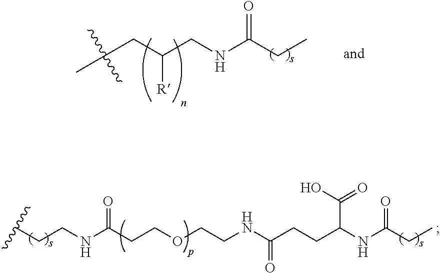
R⁸ is an amino acid side chain of a natural amino acid or an amino acid side chain of an unnatural amino acid;
R⁹ is an amino acid side chain of a natural amino acid or an amino acid side chain of an unnatural amino acid;
R¹⁰ is an amino acid side chain of a natural amino acid or an amino acid side chain of an unnatural amino acid;
m is 0 or 1;
each n, q, and u are independently an integer from 0 to 16;
each p is independently an integer from 0 to 24;
each s is independently an integer from 0 to 16;
each t is independently 1, 2, 3, 4, 5, or 6;
each R′ is independently selected from H, C(O)OH, (CH₂)OH and NHAc; and
each R″ is independently selected from H and CH₃; and
each alpha-carbon atom on the peptide backbone is optionally substituted with methyl;
wherein when a variable group R⁰, R¹, R², R³, R⁴, R⁵, R⁶, R⁷, R⁸, R⁹, or R¹⁰ is defined as the amino acid side chain of a cyclic amino acid, the corresponding amino acid nitrogen of the peptide backbone of Formula B forms part of the cyclic group;
wherein the cyclic peptide of Formula B is substituted by at least one chelator; and
wherein the cyclic peptide optionally comprises a radionuclide.

2. The cyclic peptide of claim 1, wherein the cyclic peptide of Formula B is a cyclic peptide of Formula Ia or Formula Ib: 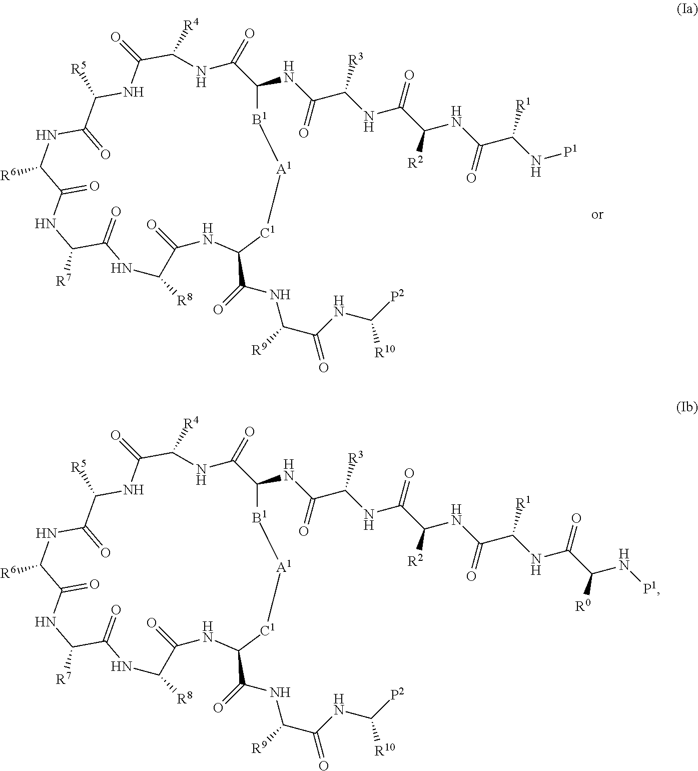
or a pharmaceutically acceptable salt thereof.

3. (canceled)

4. The cyclic peptide of claim 1, or a pharmaceutically acceptable salt thereof, wherein
P¹ is selected from Ac, 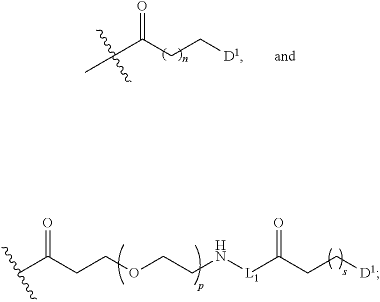
wherein:
D′ is CH₃ or C(O)OH;
L¹ is absent or 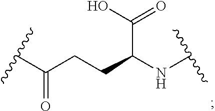 [SMILES: *N[C@@H](CCC(*)=O)C(=O)O]
n and s are independently an integer from 2 to 15; and
p is 8, 9, 10, 11, or 12.

5. (canceled)

6. The cyclic peptide of claim 1, or a pharmaceutically acceptable salt thereof, wherein
P¹ is selected from H, Ac, 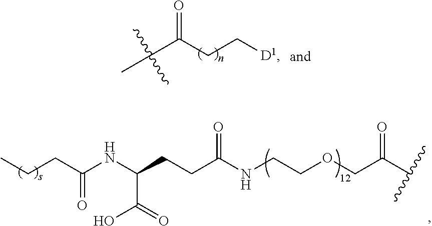
wherein:
n and s are independently 9, 10, 11, 12, or 13; and
D¹ is CH₃ or C(O)OH.

7. The cyclic peptide of claim 1, or a pharmaceutically acceptable salt thereof, wherein P² is -L²-Chelator.

8. The cyclic peptide of claim 1, or a pharmaceutically acceptable salt thereof, wherein P² is

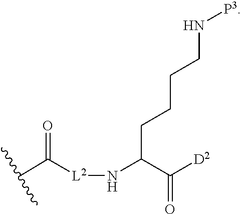

9. The cyclic peptide of claim 8, or a pharmaceutically acceptable salt thereof, wherein P³ is selected from DOTA, 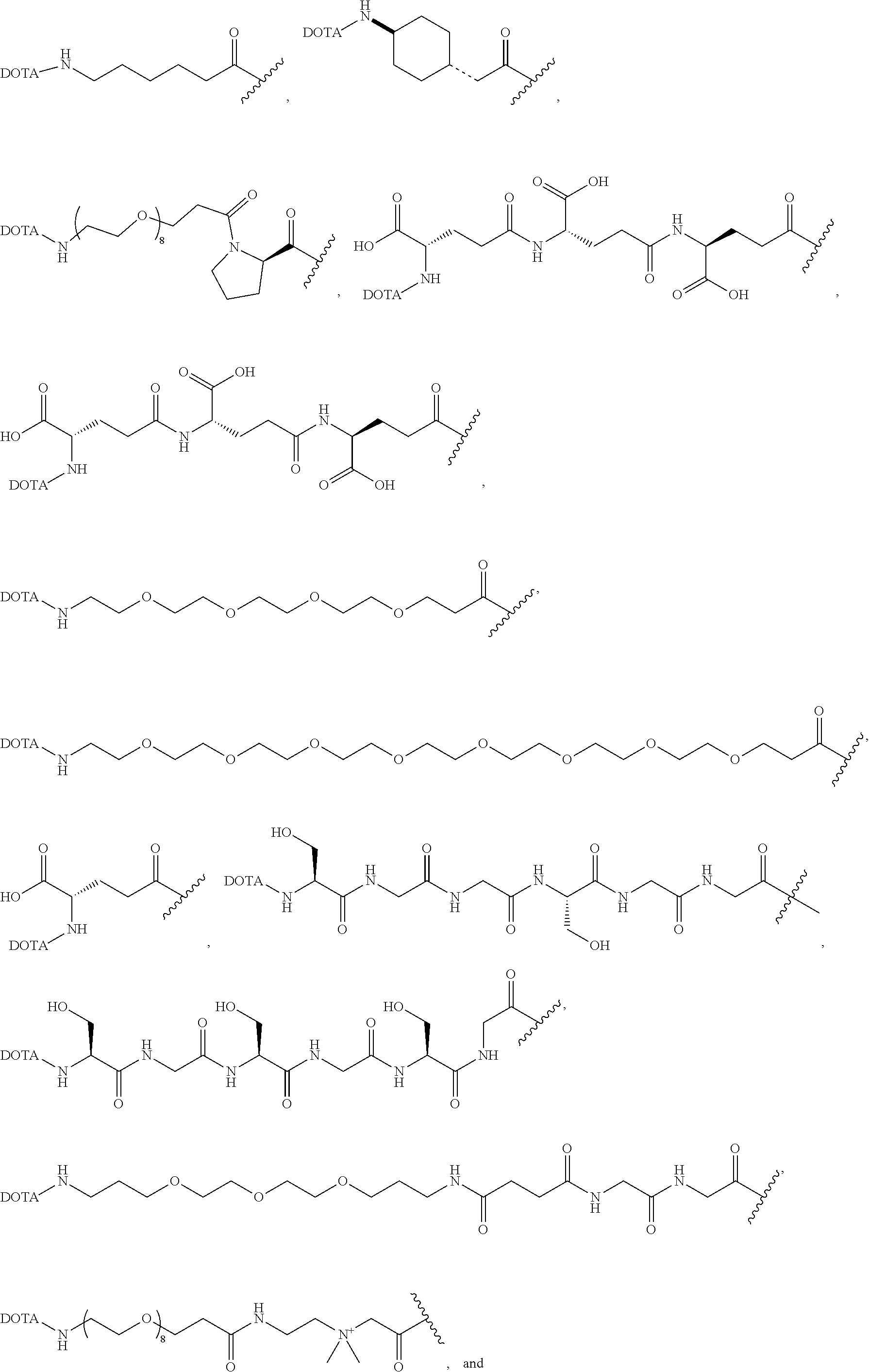 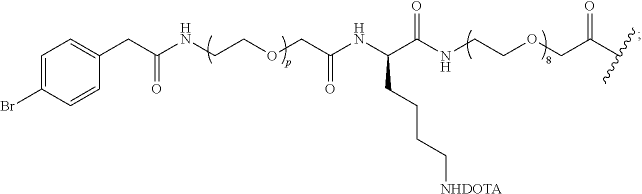
and
p is 8, 12, or 24.

10. The cyclic peptide of claim 1, wherein the cyclic peptide of Formula B is a cyclic peptide of Formula II: 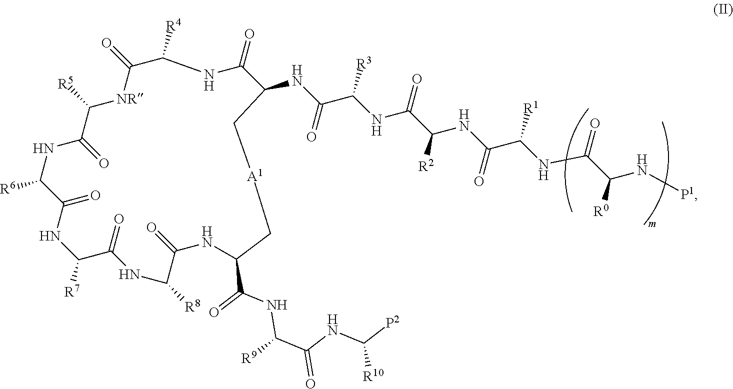
or a pharmaceutically acceptable salt thereof.

11. (canceled)

12. The cyclic peptide of claim 1, wherein
m is 0;
P¹ is selected from H, Ac, 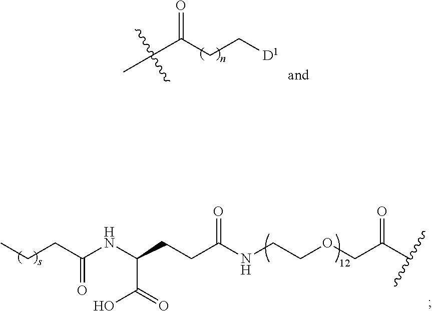
n and s are each independently 9, 10, 11, 12, or 13;
P² is
P³ is selected from DOTA, 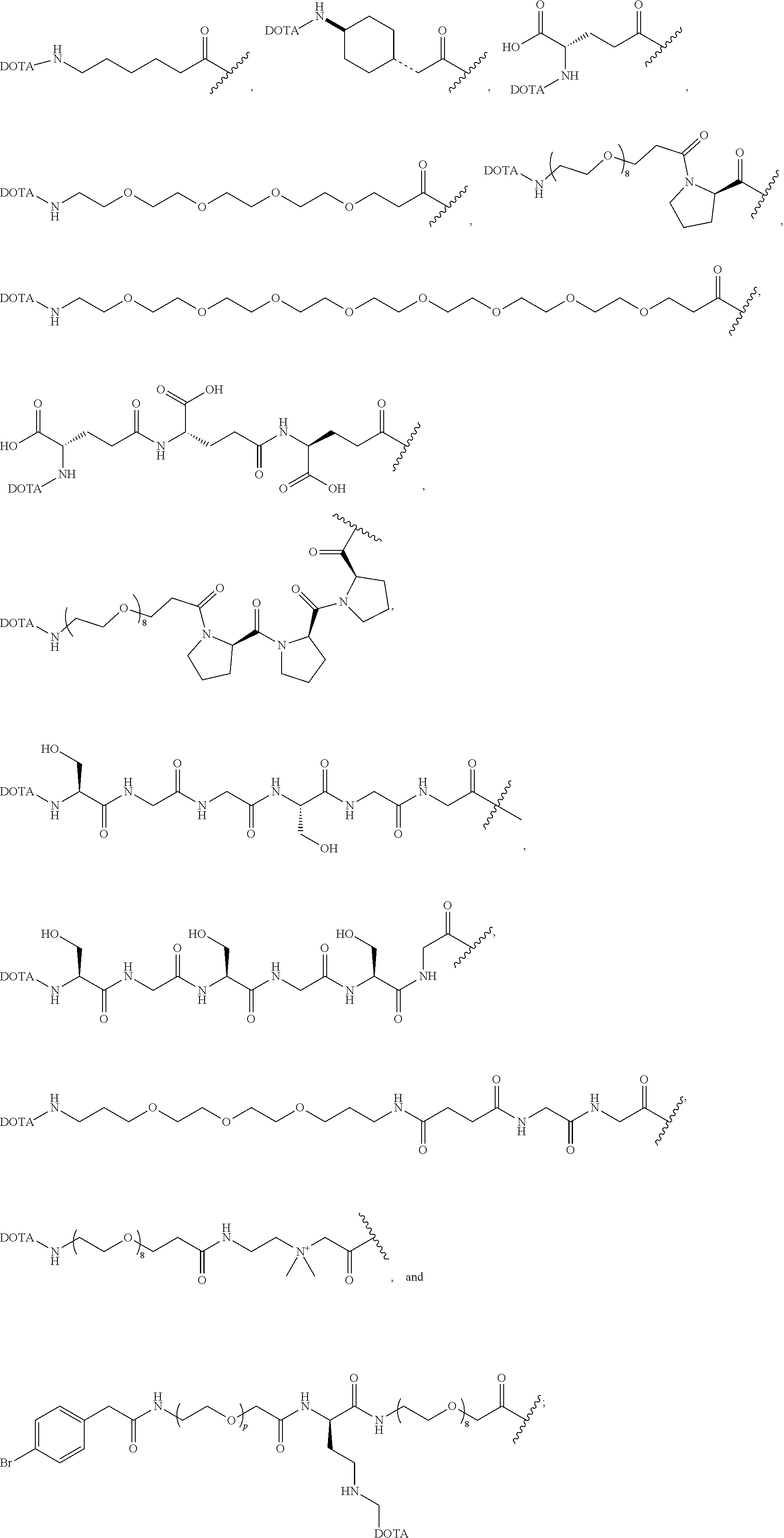
p is 8, 12, or 24;
D¹ is CH₃ or C(O)OH;
R¹ is selected from the group consisting of an amino acid side chain of Trp, 2Nal, 1Nal, 4CF₃-Phe, 7Aza-Trp, 1Me-Trp, 5OH-Trp, BIP, 5OMe-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is selected from the group consisting of an amino acid side chain of Thr, D-Ala, Ala, alpha-Me-Thr, Lys, and NMe-Thr;
R³ an amino acid side chain selected from the group consisting of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, a-tert-amylGly, AIlo-Ile, Lys(C12), Lys(C14), Lys(C16), alpha-Me-Ile, and NMe-tBuAla;
A¹ is selected from the group consisting of: 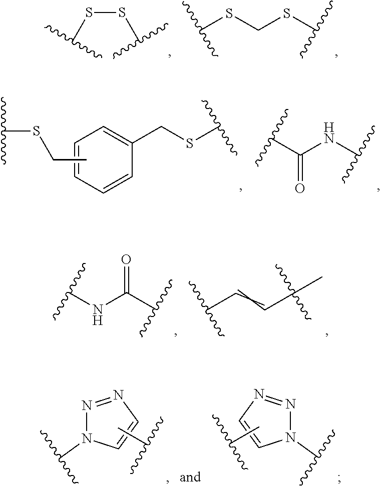
R⁴ is selected from the group consisting of an amino acid side chain of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, NMe-Asn, Pip(CH₂CO₂H)Ala, and Pip(PegNMe₃)Ala;
R⁵ is selected from the group consisting of an amino acid side chain of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is selected from the group consisting of an amino acid side chain of Trp, 4CF₃-Phe, 1Me-Trp, 7Aza-Trp, BIP, 2Nal, 1Nal, alpha-Me-Trp, D-Ala, Ala, 4F-Phe, 5F-Trp, 5Ome-Trp, Asn, 5OH-Trp, 7Me-Trp, 7MeO-Trp, 7Cl-Trp, and NMe-Trp;
R⁷ is:
(i) an amino acid side chain selected from the group consisting of 3Pya, 4Pya, Lys(Me)3, His, Ala, D-Ala, Gln, Lys, Glu, Arg, Orn, NMe-His, and Ser; or
(ii) selected from the group consisting of: 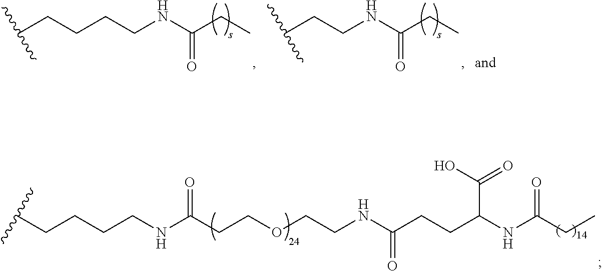
each s is independently 3, 5,10, 12, or 14;
R⁸ is selected from the group consisting of an amino acid side chain of Asp, D-Ala, Ala, Asn, Thr, NMe-Asp, and alpha-Me-Asp;
R⁹ is selected from the group consisting of an amino acid side chain of Trp, 7Aza-Trp, 1Me-Trp, D-Ala, Ala, 4F-Phe, 1Nal, 2Nal, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, D-Ala, Ala, alpha-Me-Pro, trans4Fluoro-Pro, cis4Fluoro-Pro, trans40H-Pro, cis4OH-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACI, and 3Me2-Aze.

13. The cyclic peptide of claim 1, or a pharmaceutically acceptable salt thereof, wherein
m is 1;
P¹ is selected from Ac, 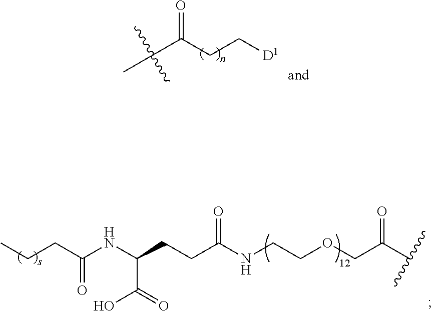
n and s are each independently 9, 10, 11, 12, or 13;
P² is 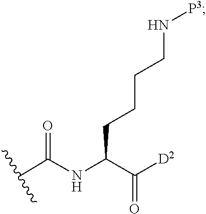
P³ is selected from DOTA, 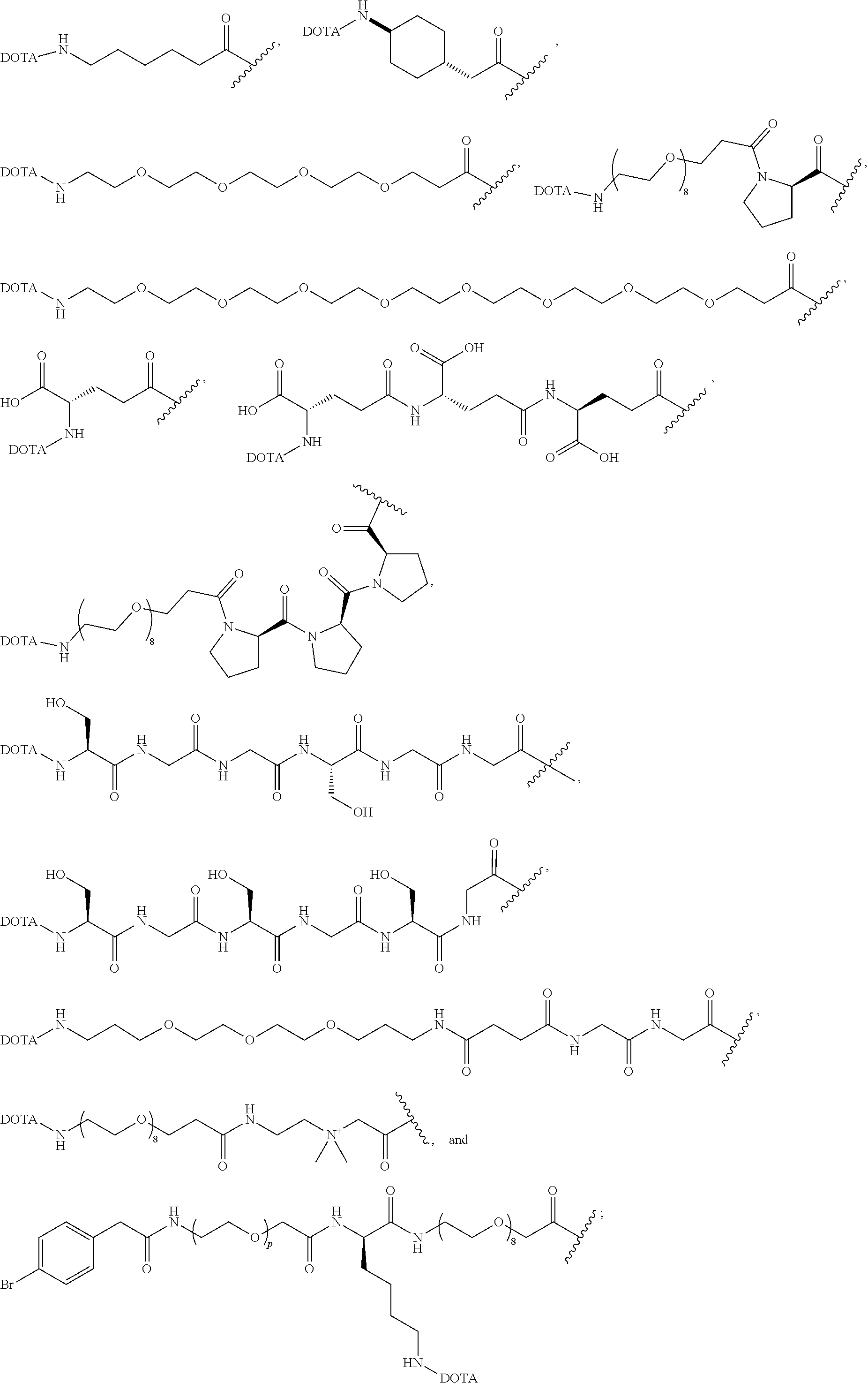
p is 8, 12, or 24;
D¹ is CH₃ or C(O)OH;
R⁰ is selected from the group consisting of an amino acid side chain of Gly, Met, D-Ala, Ala, Nle, and Nva;
R¹ is selected from the group consisting of an amino acid side chain of Trp, 2Nal, 1Nal, 4CF₃-Phe, 7Aza-Trp, 1Me-Trp, 5OH-Trp, BIP, 5Ome-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is selected from the group consisting of an amino acid side chain of Thr, D-Ala, Ala, alpha-Me-Thr, Lys, and NMe-Thr;
R³ is selected from the group consisting of an amino acid side chain of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of 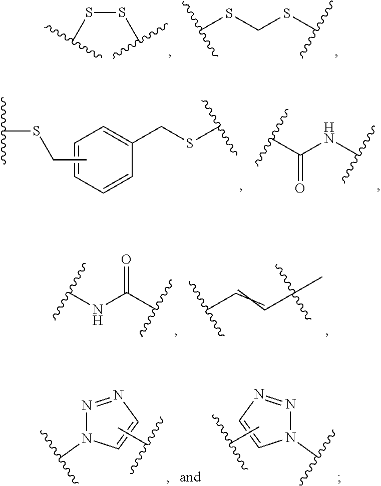
R⁴ is selected from the group consisting of an amino acid side chain of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, and NMe-Asn;
R⁵ is selected from the group consisting of an amino acid side chain of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is selected from the group consisting of an amino acid side chain of Trp, 4CF₃-Phe, 1Me-Trp, 7Aza-Trp, BIP, 2Nal, 1Nal, alpha-Me-Trp, D-Ala, Ala, 4F-Phe, 5F-Trp, 5OMe-Trp, Asn, 5OH-Trp, 7Me-Trp, 7MeO-Trp, 7Cl-Trp, and NMe-Trp;
R⁷ is selected from the group consisting of an amino acid side chain of 3Pya, 4Pya, Lys(Me)₃, His, Ala, D-Ala, Gln, Lys, Glu, Arg, Orn, NMe-His, and Ser; or
R⁷ is selected from the group consisting of 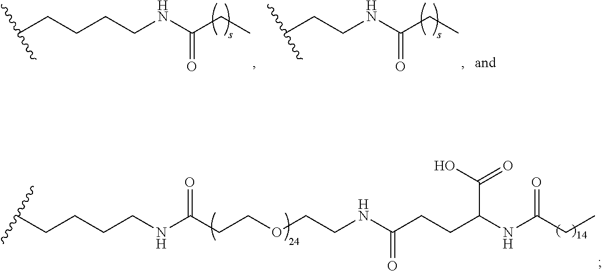
each s is independently 3, 5,10, 12, or 14;
R⁸ is selected from the group consisting of an amino acid side chain of Asp, D-Ala, Ala, Asn, Thr, NMe-Asp, and alpha-Me-Asp;
R⁹ is selected from the group consisting of an amino acid side chain of Trp, 7Aza-Trp, 1Me-Trp, D-Ala, Ala, 4F-Phe, 1Nal, 2Nal, 5F-Trp, 5Ome-Trp, alpha-Me-Trp, 7Cl-Trp, 5OH-Trp, 7Me-Trp, 7MeO-Trp, and NMe-Trp; and
R¹⁰ is selected from the group consisting of an amino acid side chain of Pro, D-Ala, Ala, alpha-Me-Pro, trans4Fluoro-Pro, cis4Fluoro-Pro, trans40H-Pro, cis4OH-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACI, and 3Me2-Aze.

14. The cyclic peptide of claim 1, or a pharmaceutically acceptable salt thereof, wherein
B¹ is CH₂ or C(CH₃)₂; and
C¹ is CH₂ or C(CH₃)₂.

15. (canceled)

16. The cyclic peptide of claim 1, or a pharmaceutically acceptable salt thereof, wherein Chelator is independently selected from a group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N, N′,N″,N′″,N″″,N′″″-hexaacetic acid (HEHA).

17. (canceled)

18. The cyclic peptide of claim 1, or a pharmaceutically acceptable salt thereof, wherein the cyclic peptide of Formula B, or a pharmaceutically acceptable salt thereof, is selected from

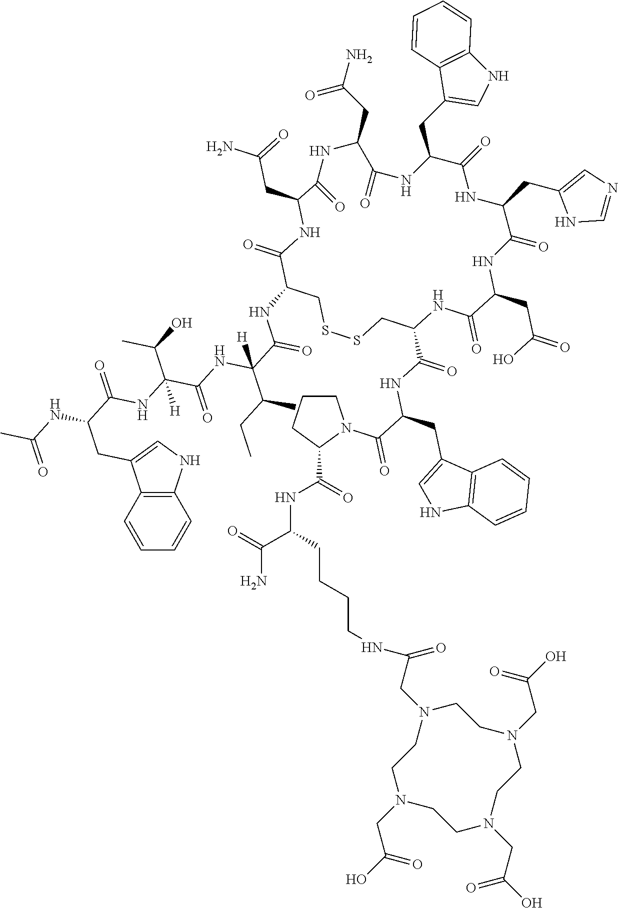 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

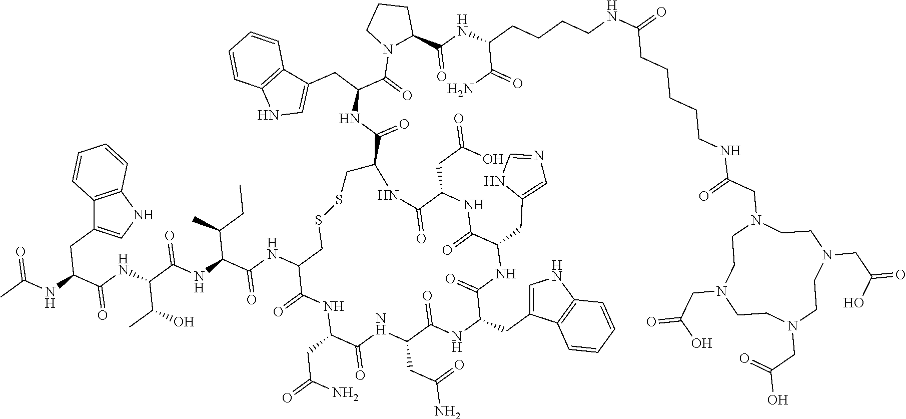 [SMILES: CC[C@H](C)[C@H](NC(=O)[C@@H](NC(=O)[C@H](Cc1c[nH]c2ccccc12)NC(C)=O)[C@@H](C)O)C(=O)NC1CSSC[C@@H](C(=O)N[C@@H](Cc2c[nH]c3ccccc23)C(=O)N2CCC[C@H]2C(=O)N[C@H](CCCCNC(=O)CCCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

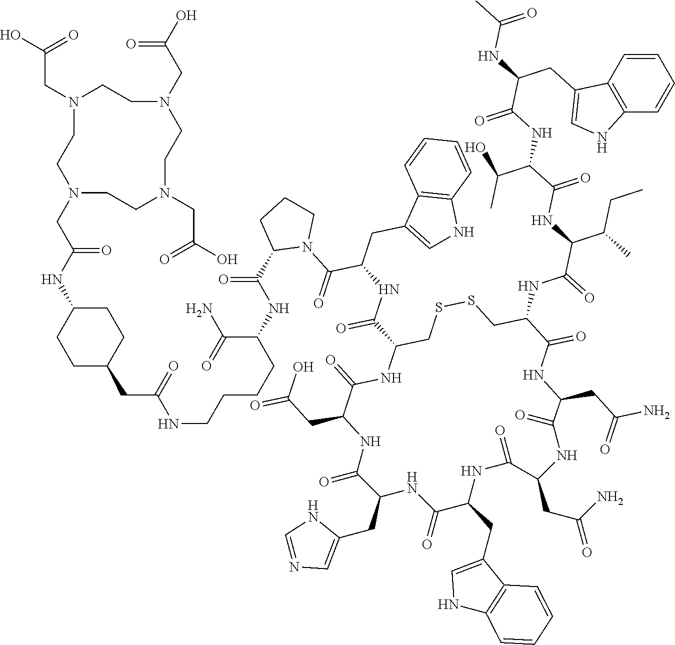 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)C[C@H]2CC[C@H](NC(=O)CN3CCN(CC(=O)O)CCN(CC(=O)O)CCN(CC(=O)O)CC3)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

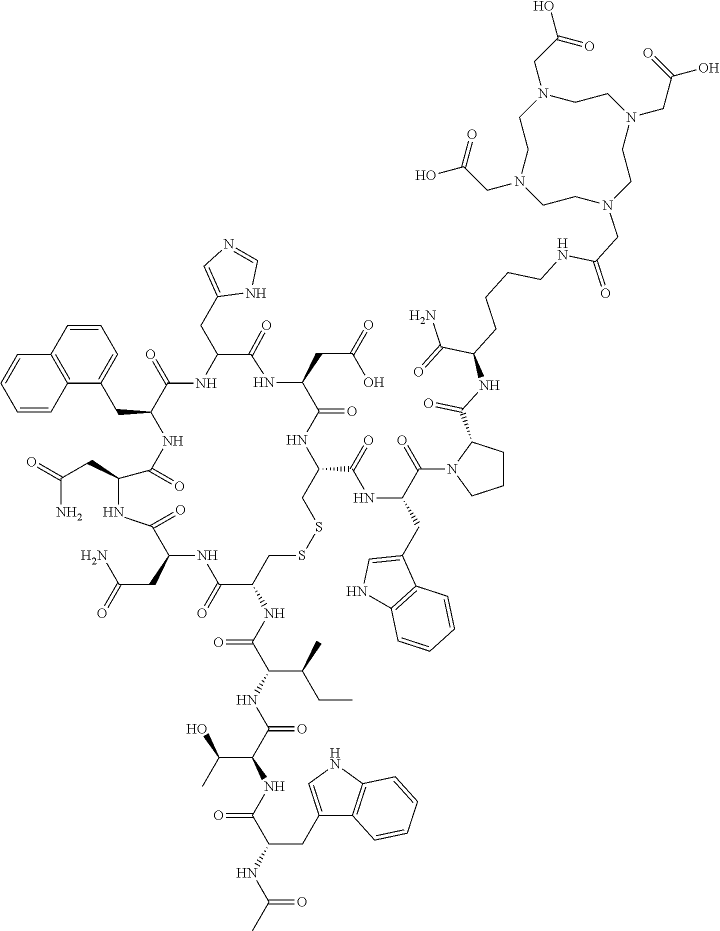 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)C(Cc2cnc[nH]2)NC(=O)[C@H](Cc2cccc3ccccc23)NC(=O)[C@H](CC(N)=O)NC(=O)[C@H](CC(N)=O)NC1=O]

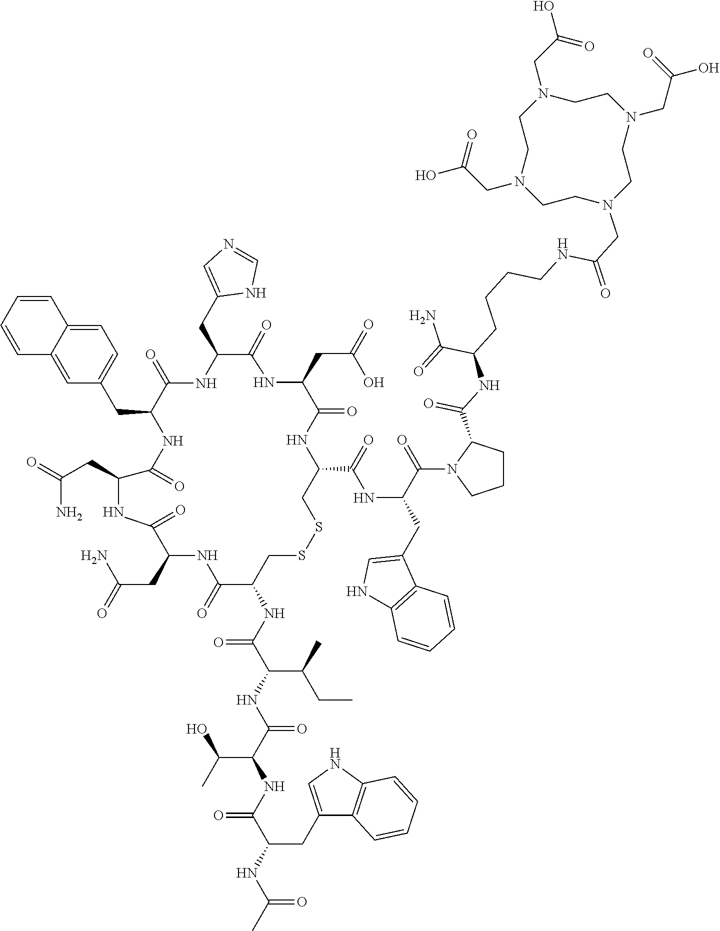 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

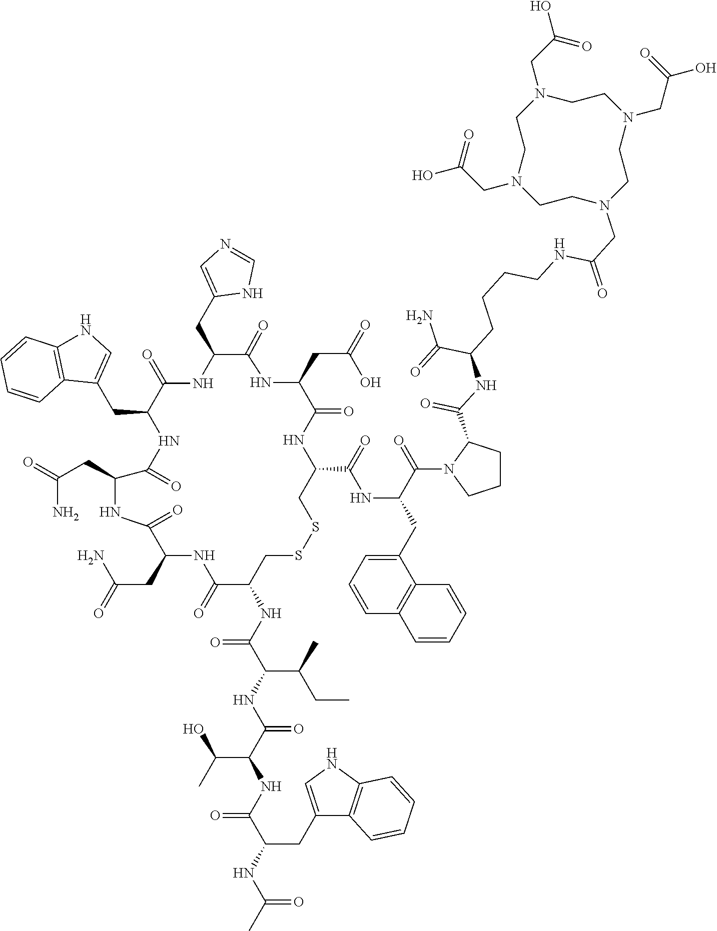 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2cccc3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

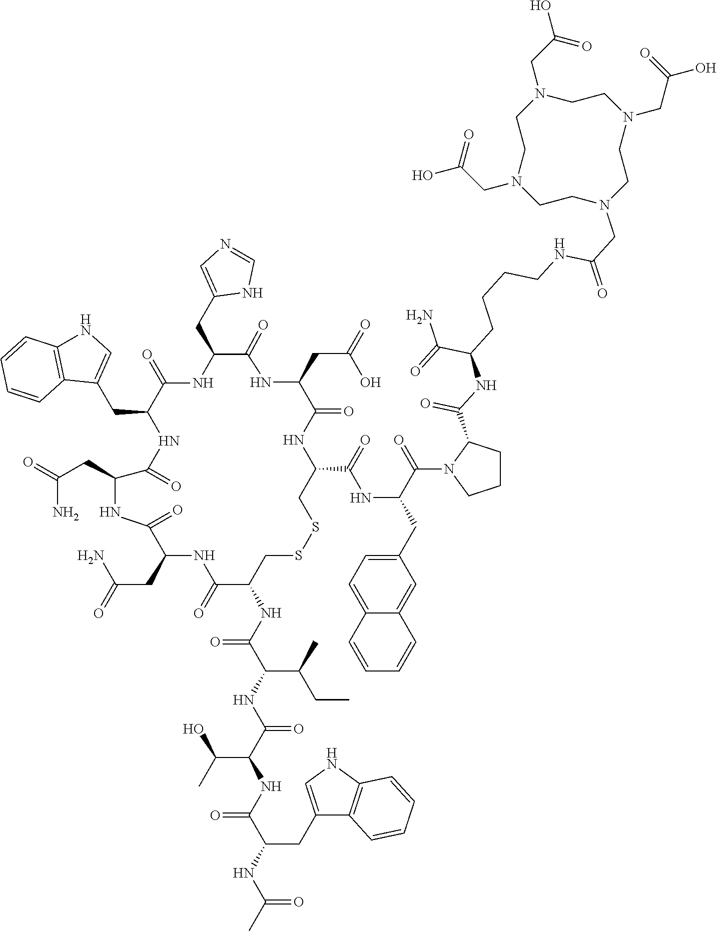 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2ccc3ccccc3c2)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

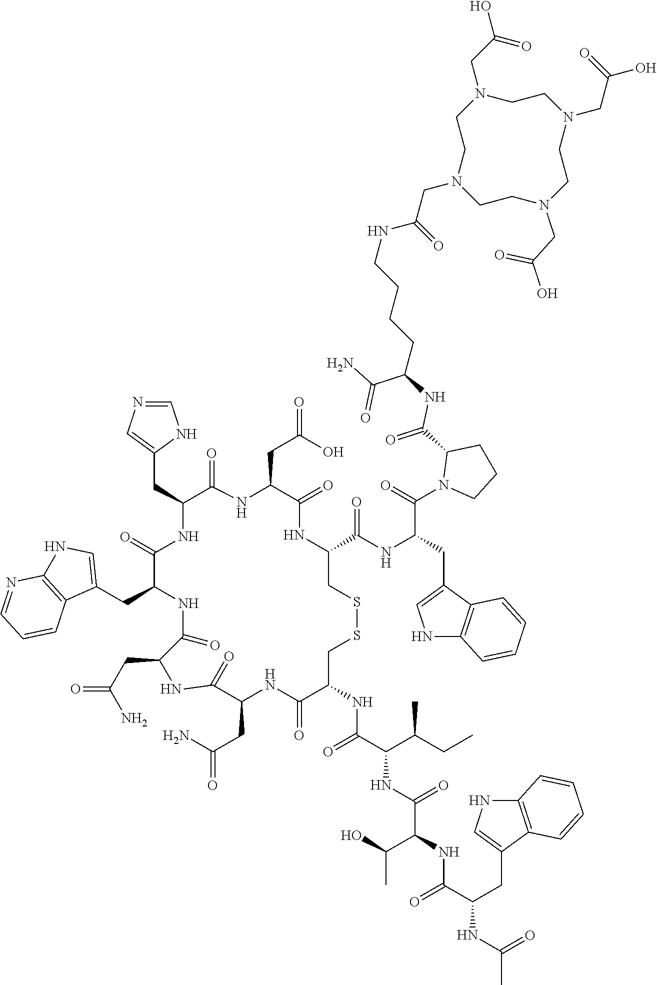 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ncccc23)NC(=O)[C@H](CC(N)=O)NC(=O)[C@H](CC(N)=O)NC1=O]

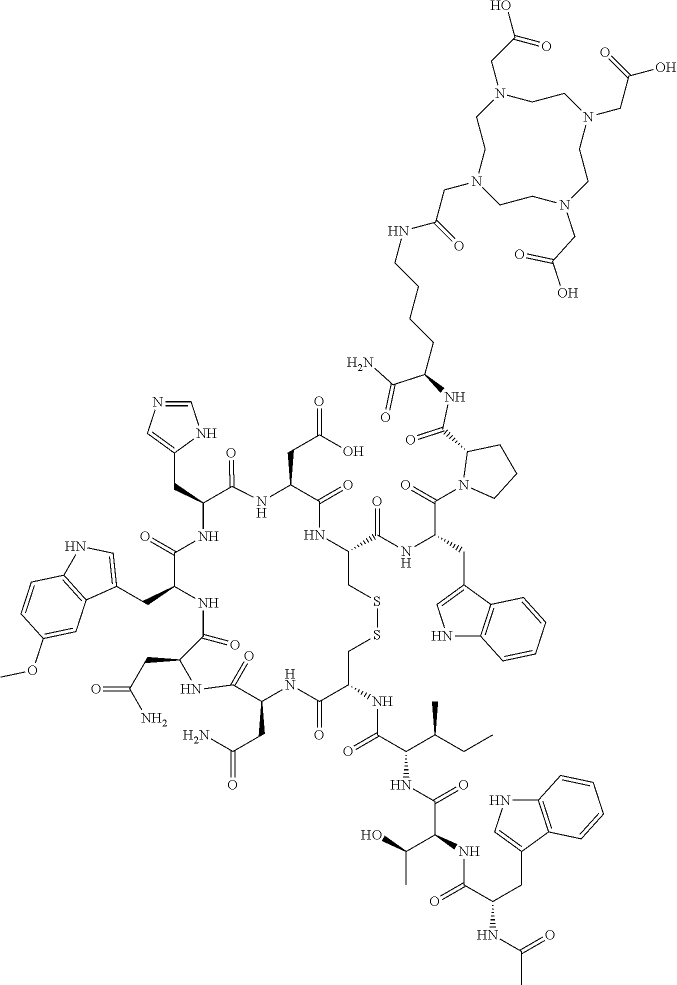 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccc(OC)cc23)NC(=O)[C@H](CC(N)=O)NC(=O)[C@H](CC(N)=O)NC1=O]

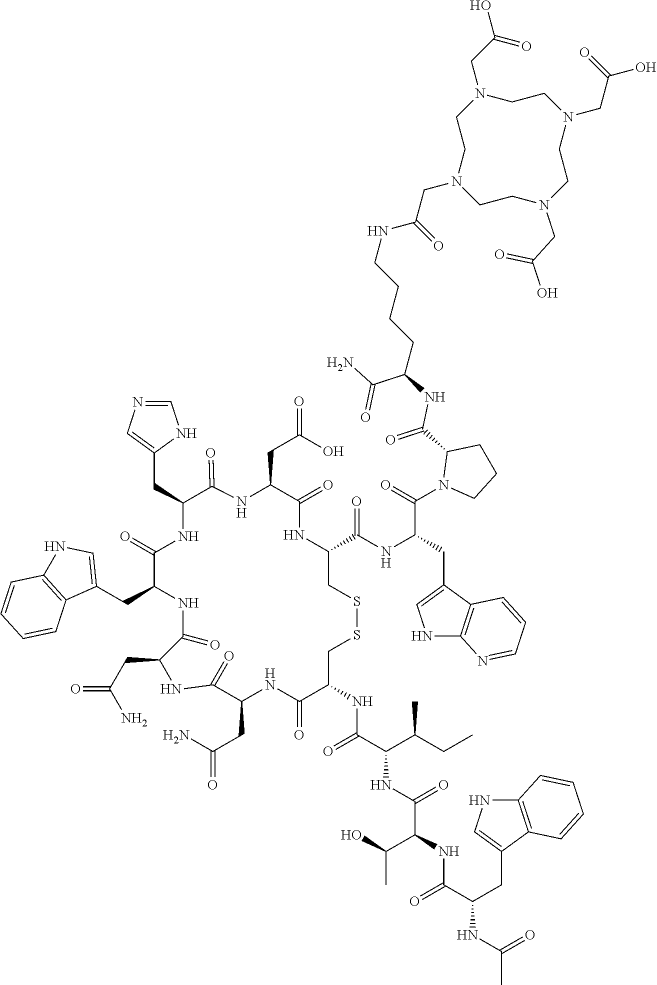 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

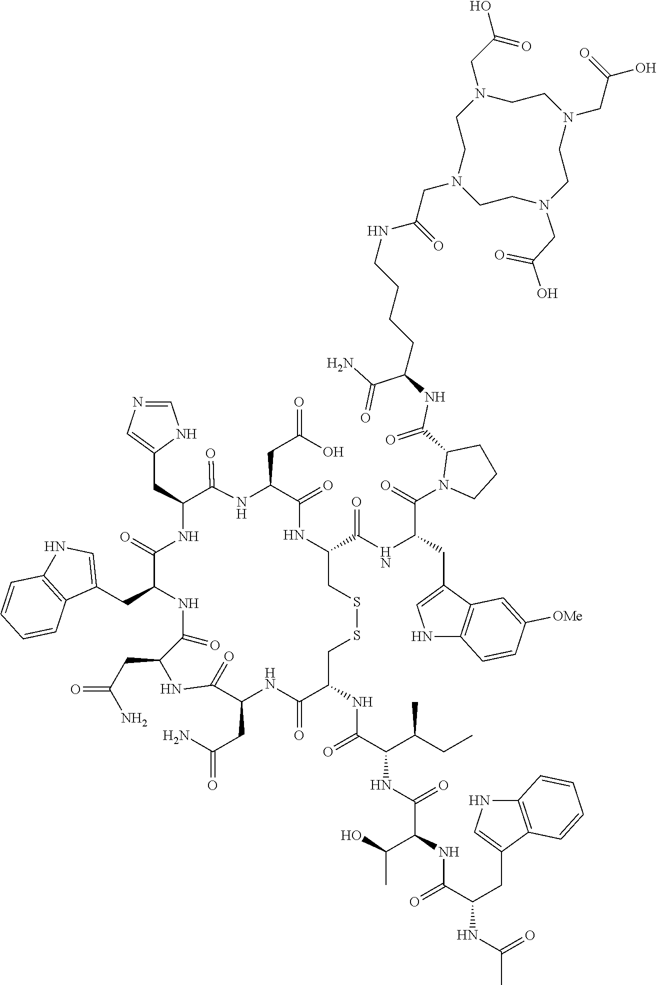 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccc(OC)cc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

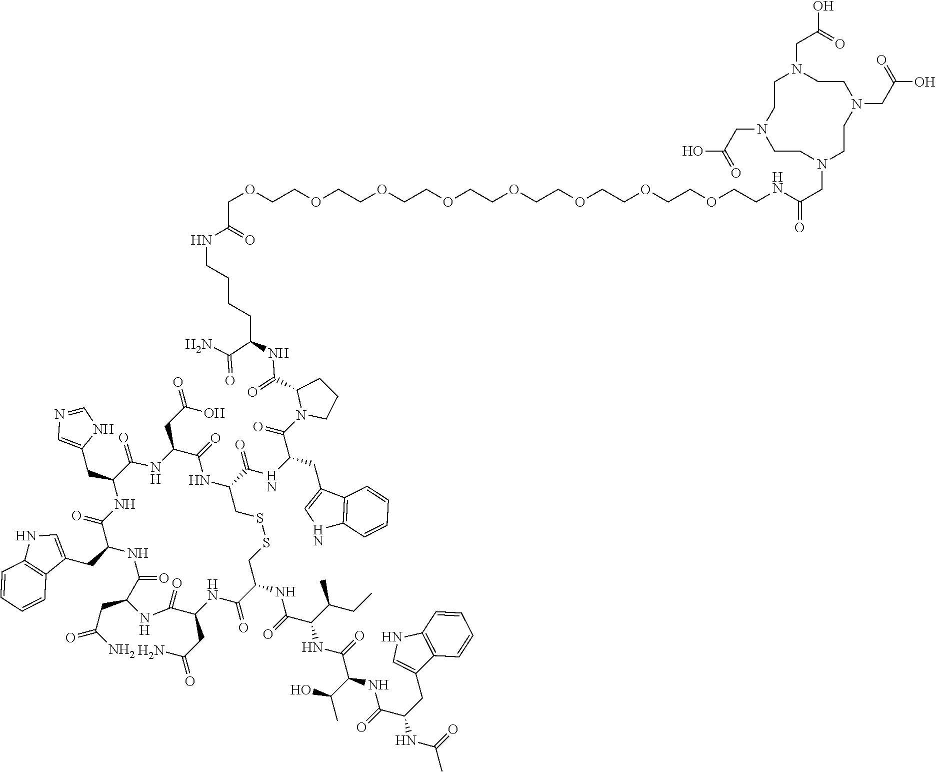 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)COCCOCCOCCOCCOCCOCCOCCOCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

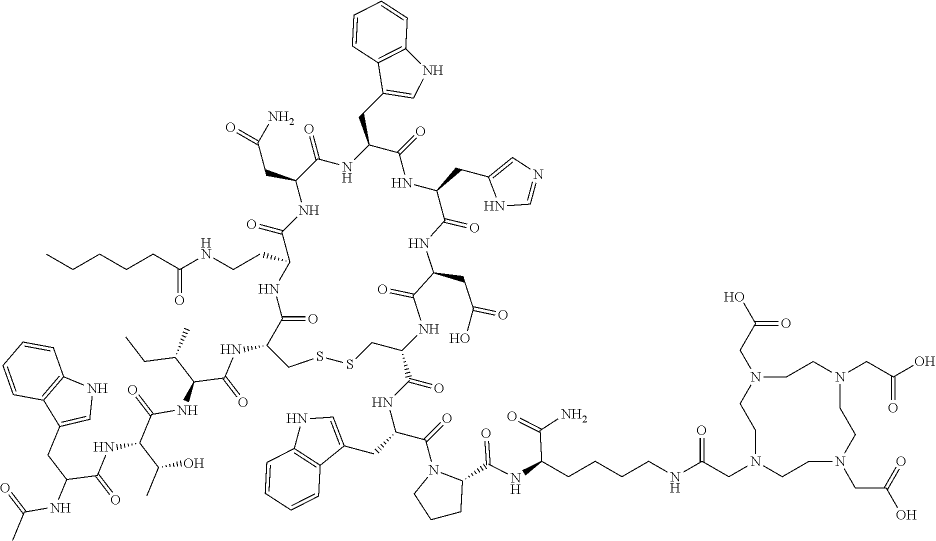 [SMILES: CCCCCC(=O)NCC[C@@H]1NC(=O)[C@@H](NC(=O)[C@@H](NC(=O)[C@@H](NC(=O)C(Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC1=O]

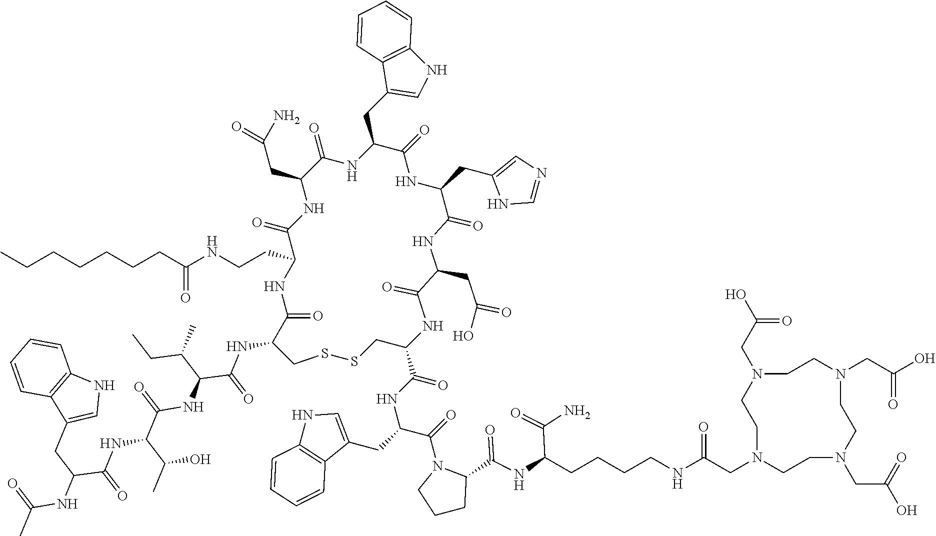 [SMILES: CCCCCCCC(=O)NCC[C@@H]1NC(=O)[C@@H](NC(=O)[C@@H](NC(=O)[C@@H](NC(=O)C(Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC1=O]

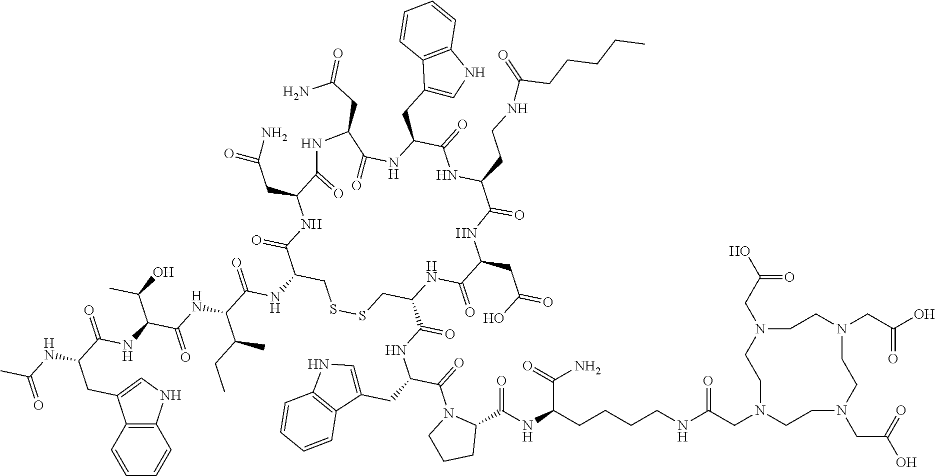 [SMILES: CCCCCC(=O)NCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O]

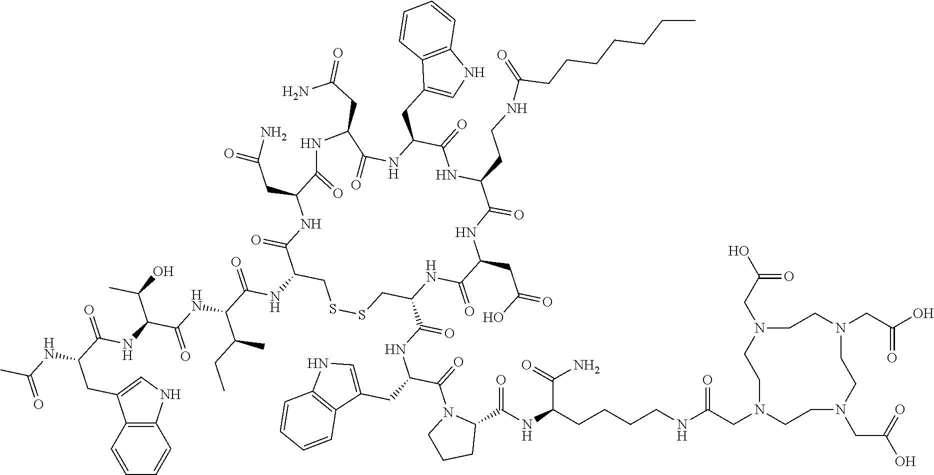 [SMILES: CCCCCCCC(=O)NCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O]

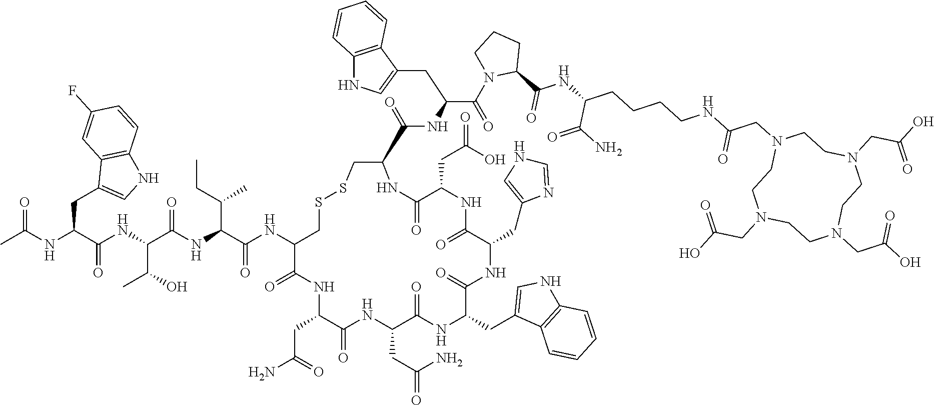 [SMILES: CC[C@H](C)[C@H](NC(=O)[C@@H](NC(=O)[C@H](Cc1c[nH]c2ccc(F)cc12)NC(C)=O)[C@@H](C)O)C(=O)NC1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

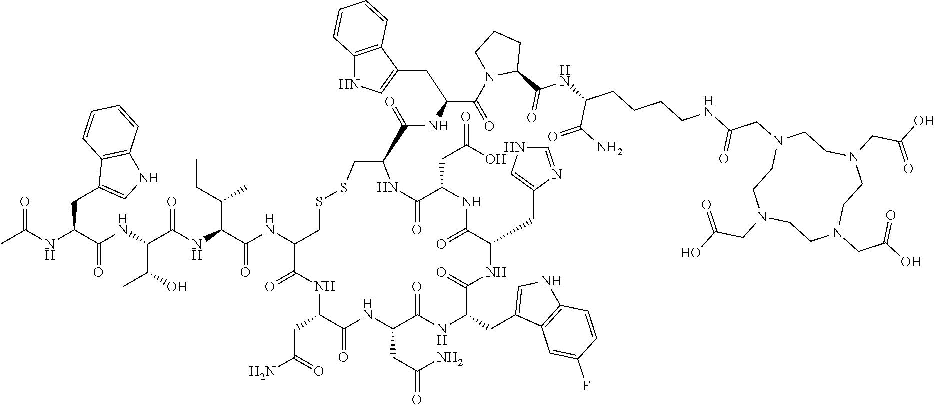 [SMILES: CC[C@H](C)[C@H](NC(=O)[C@@H](NC(=O)[C@H](Cc1c[nH]c2ccccc12)NC(C)=O)[C@@H](C)O)C(=O)NC1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccc(F)cc23)NC(=O)[C@H](CC(N)=O)NC(=O)[C@H](CC(N)=O)NC1=O]

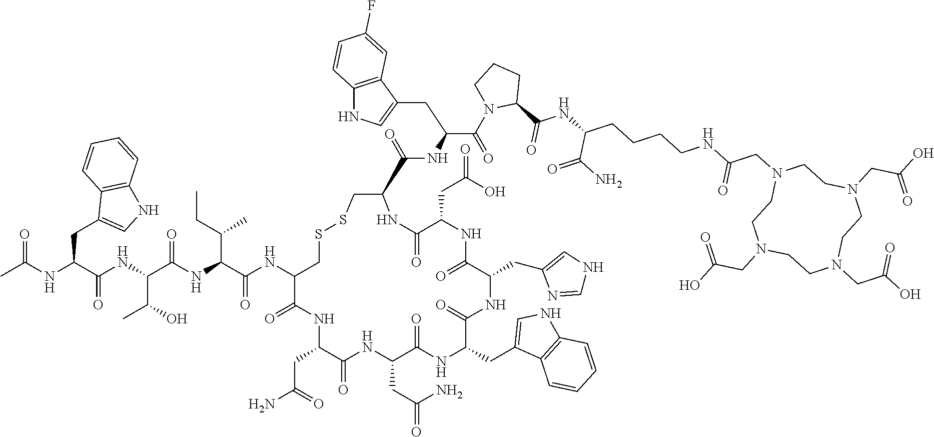 [SMILES: CC[C@H](C)[C@H](NC(=O)[C@@H](NC(=O)[C@H](Cc1c[nH]c2ccccc12)NC(C)=O)[C@@H](C)O)C(=O)NC1CSSC[C@@H](C(=O)N[C@@H](Cc2c[nH]c3ccc(F)cc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

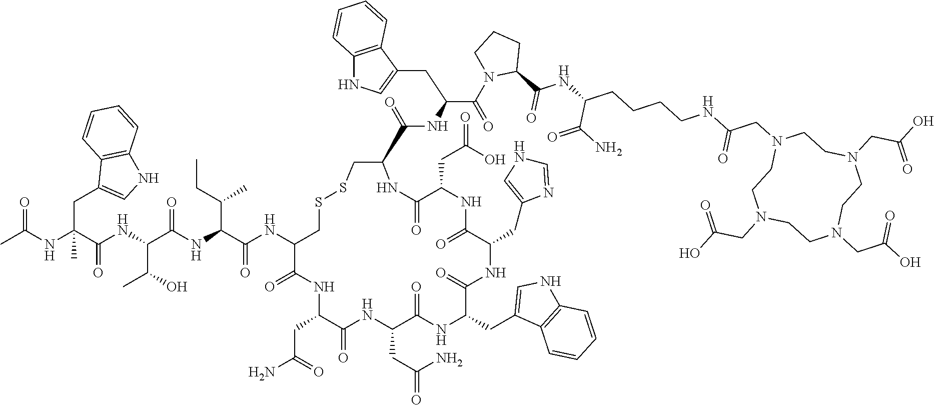 [SMILES: CC[C@H](C)[C@H](NC(=O)[C@@H](NC(=O)[C@](C)(Cc1c[nH]c2ccccc12)NC(C)=O)[C@@H](C)O)C(=O)NC1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

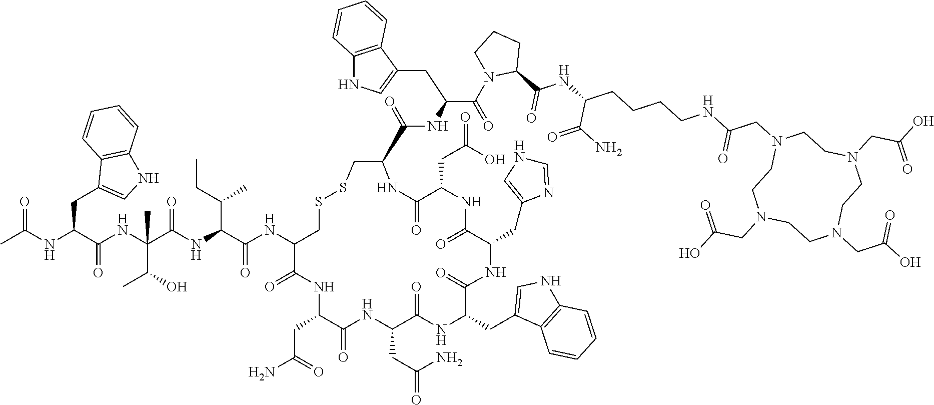 [SMILES: CC[C@H](C)[C@H](NC(=O)[C@@](C)(NC(=O)[C@H](Cc1c[nH]c2ccccc12)NC(C)=O)[C@@H](C)O)C(=O)NC1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

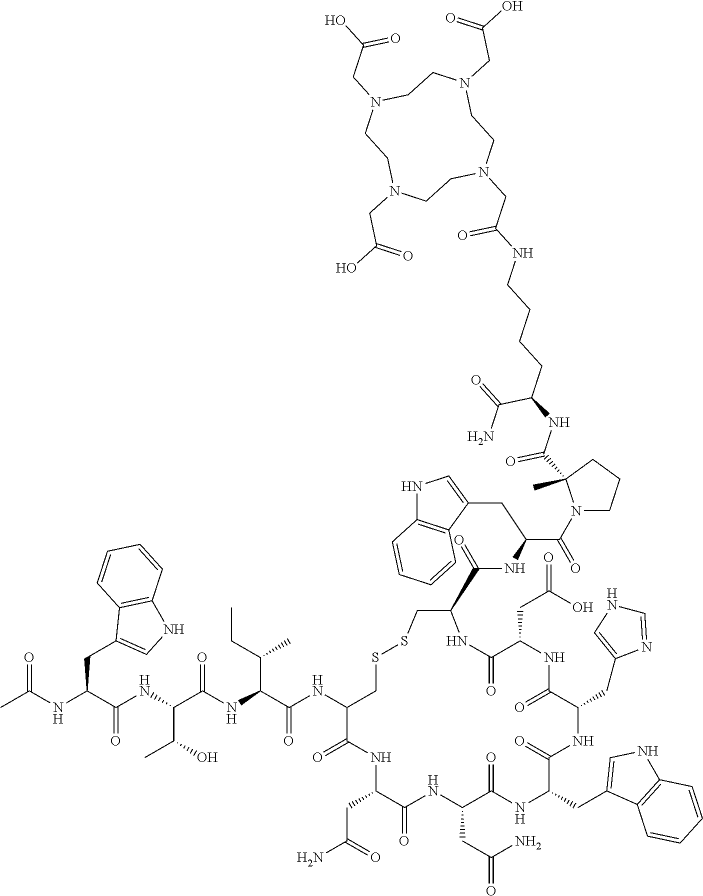 [SMILES: CC[C@H](C)[C@H](NC(=O)[C@@H](NC(=O)[C@H](Cc1c[nH]c2ccccc12)NC(C)=O)[C@@H](C)O)C(=O)NC1CSSC[C@@H](C(=O)N[C@@H](Cc2c[nH]c3ccccc23)C(=O)N2CCC[C@@]2(C)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

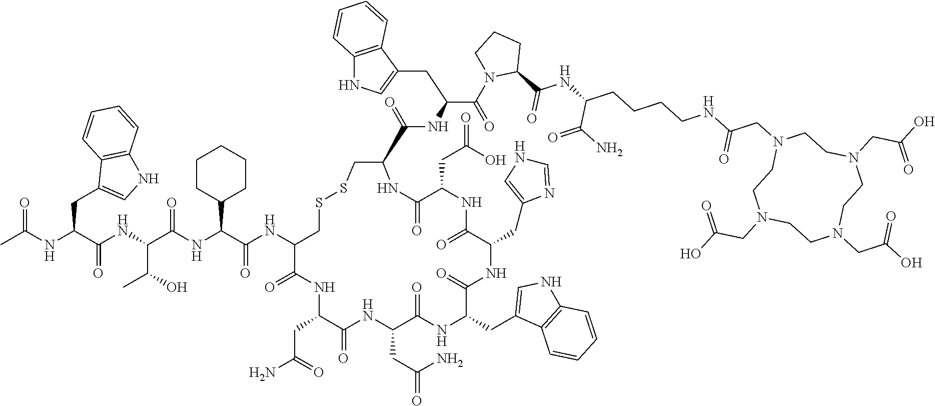 [SMILES: CC(=O)N[C@@H](Cc1c[nH]c2ccccc12)C(=O)N[C@H](C(=O)N[C@H](C(=O)NC1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O)C1CCCCC1)[C@@H](C)O]

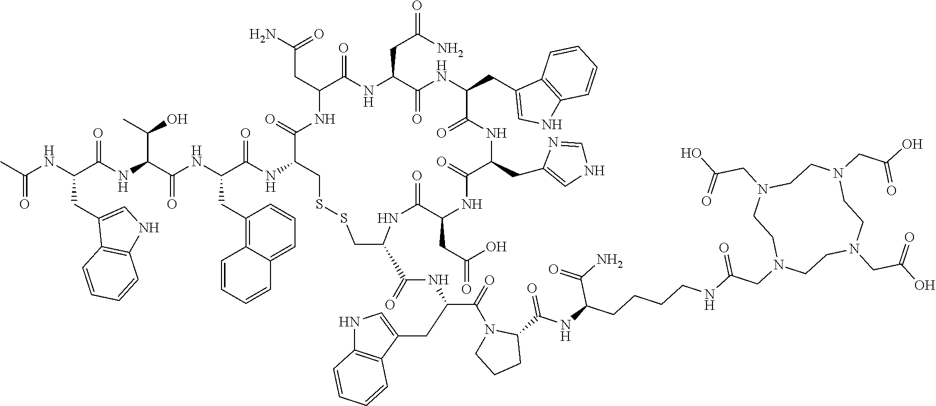 [SMILES: CC(=O)N[C@@H](Cc1c[nH]c2ccccc12)C(=O)N[C@H](C(=O)N[C@@H](Cc1cccc2ccccc12)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)C(CC(N)=O)NC1=O)[C@@H](C)O]

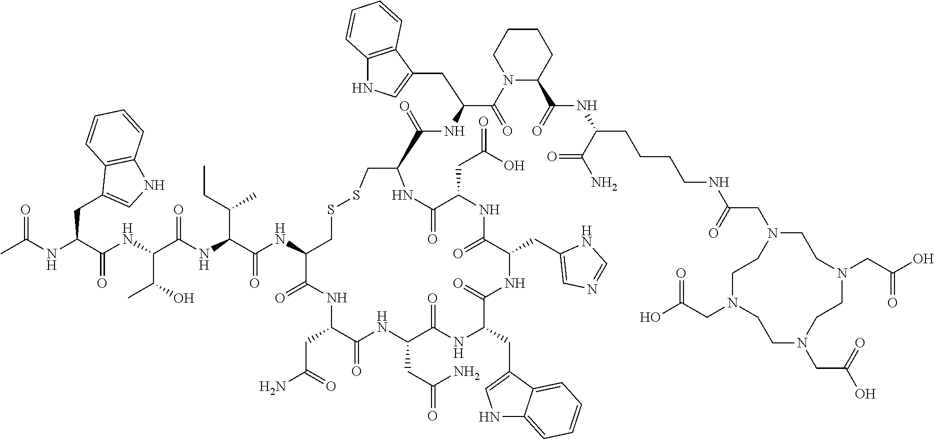 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

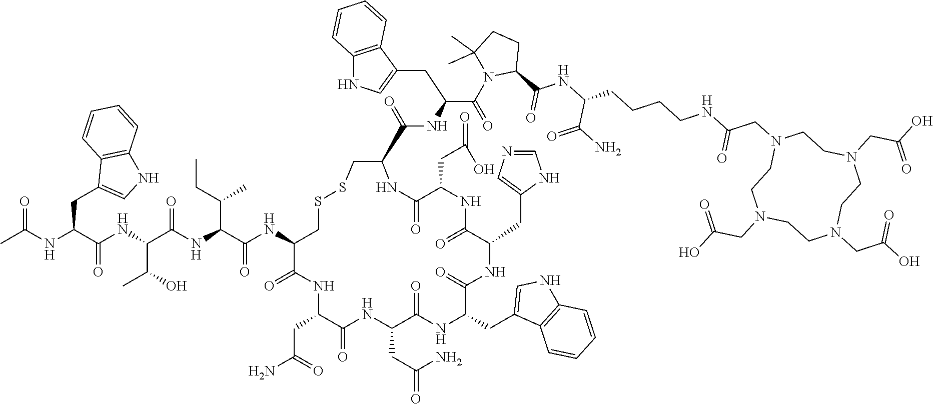 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2[C@H](C(=O)N[C@H](CCCCNC(=O)CN3CCN(CC(=O)O)CCN(CC(=O)O)CCN(CC(=O)O)CC3)C(N)=O)CCC2(C)C)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

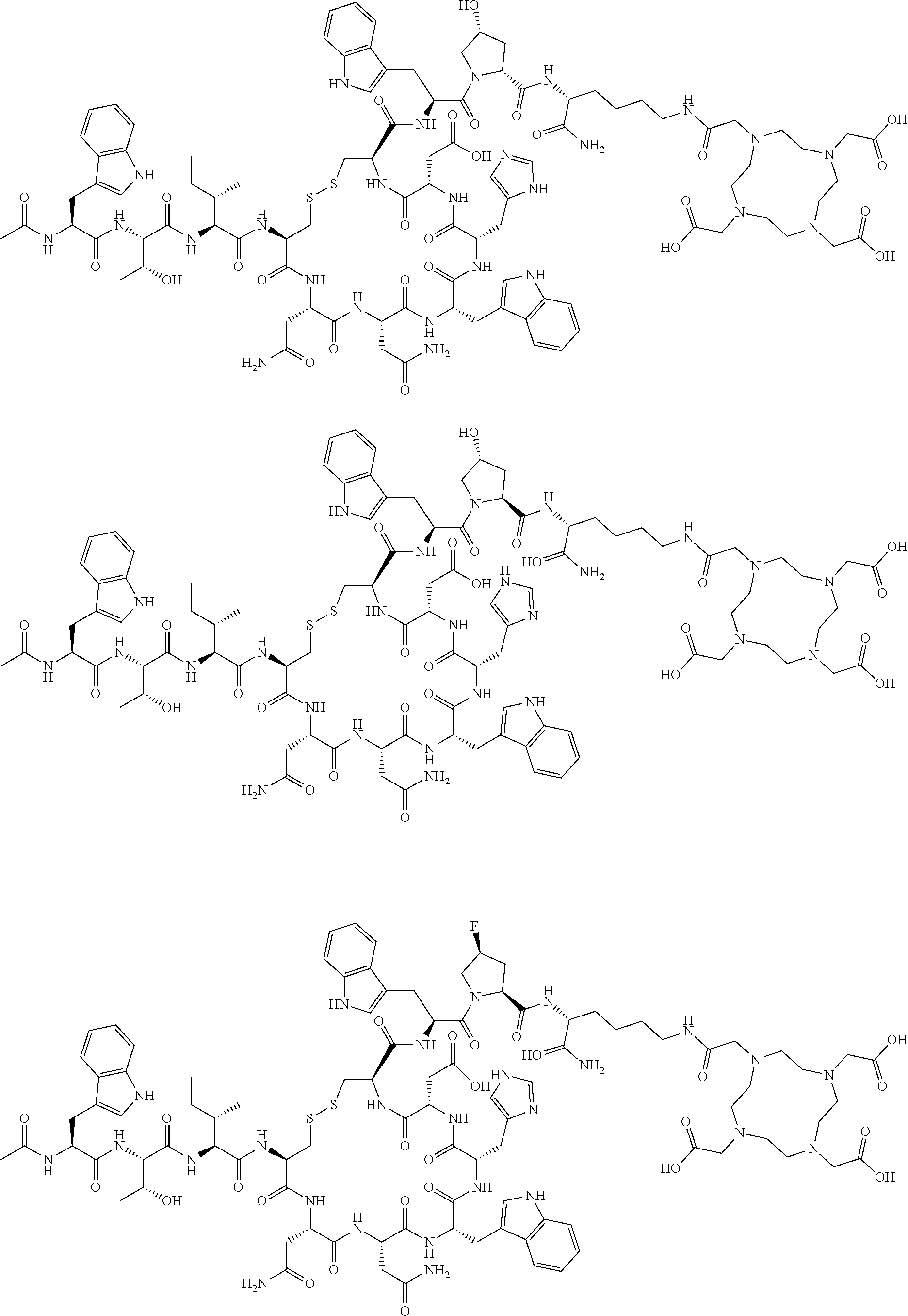

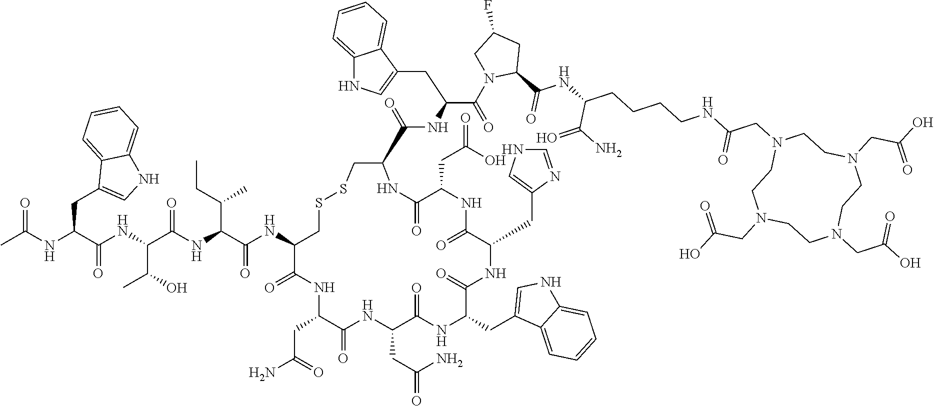 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

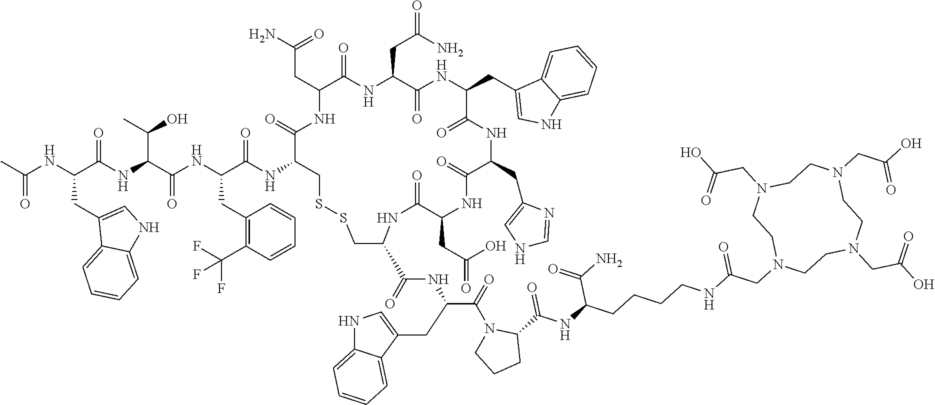 [SMILES: CC(=O)N[C@@H](Cc1c[nH]c2ccccc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)C(CC(N)=O)NC1=O)[C@@H](C)O]

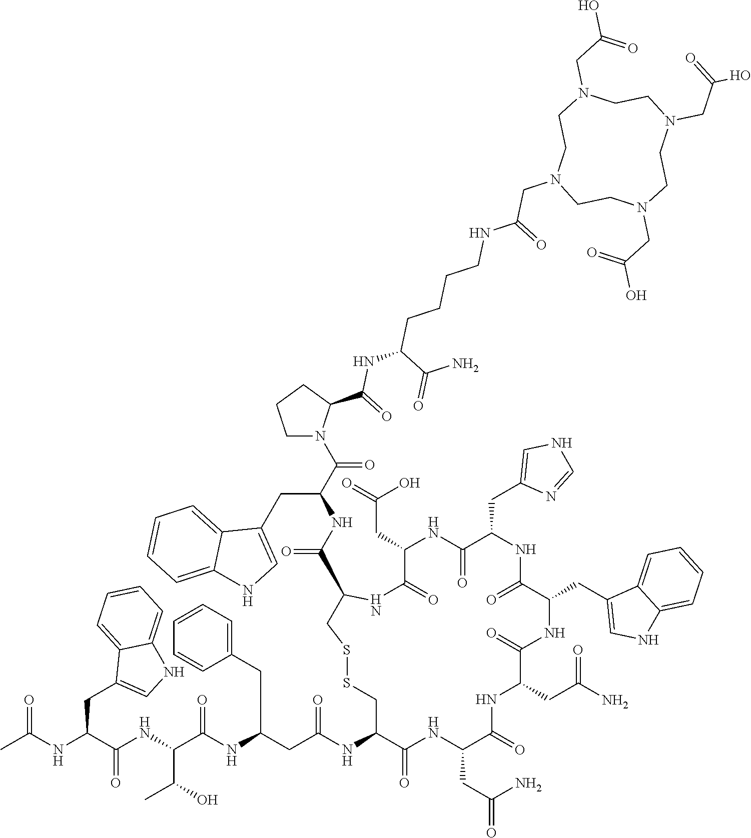 [SMILES: CC(=O)N[C@@H](Cc1c[nH]c2ccccc12)C(=O)N[C@H](C(=O)N[C@H](CC(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O)Cc1ccccc1)[C@@H](C)O]

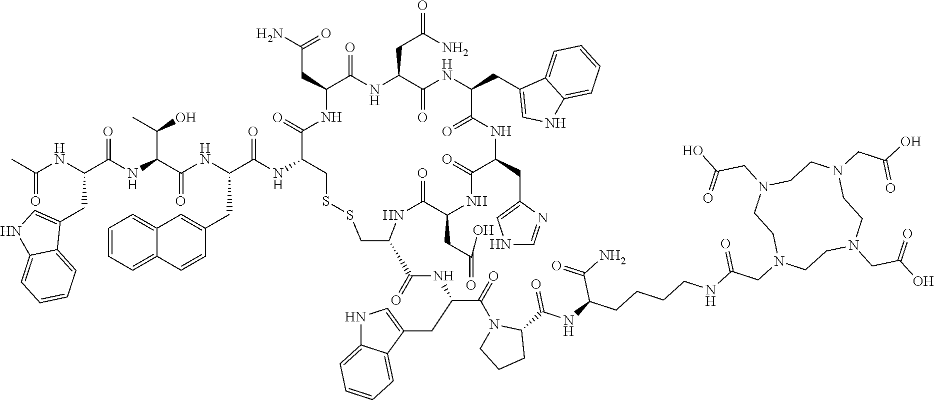 [SMILES: CC(=O)N[C@@H](Cc1c[nH]c2ccccc12)C(=O)N[C@H](C(=O)N[C@@H](Cc1ccc2ccccc2c1)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O)[C@@H](C)O]

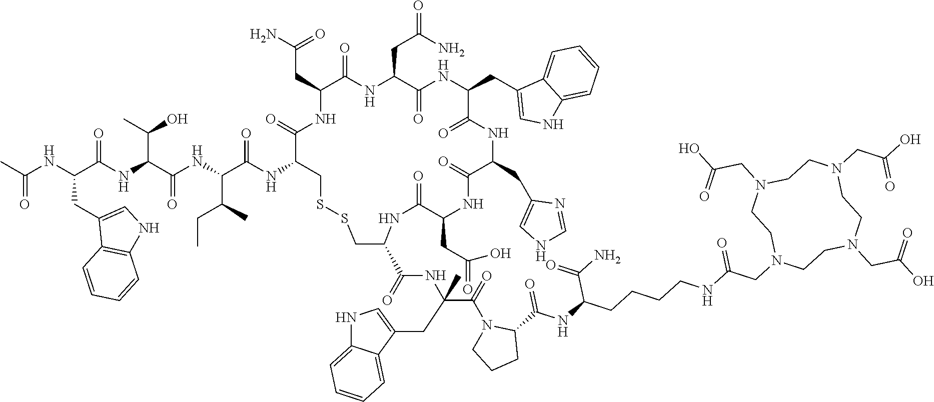 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@](C)(Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@H](Cc2c[nH]c3ccccc23)NC(=O)[C@H](CC(N)=O)NC(=O)[C@H](CC(N)=O)NC1=O]

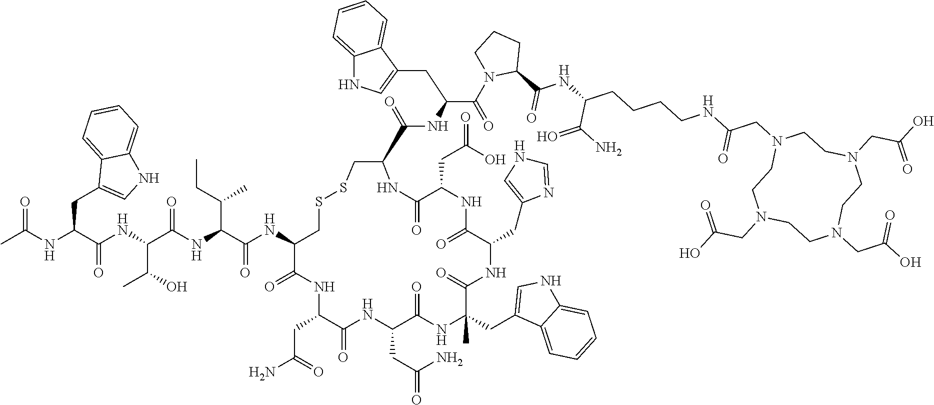 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2c[nH]cn2)NC(=O)[C@](C)(Cc2c[nH]c3ccccc23)NC(=O)[C@H](CC(N)=O)NC(=O)[C@H](CC(N)=O)NC1=O]

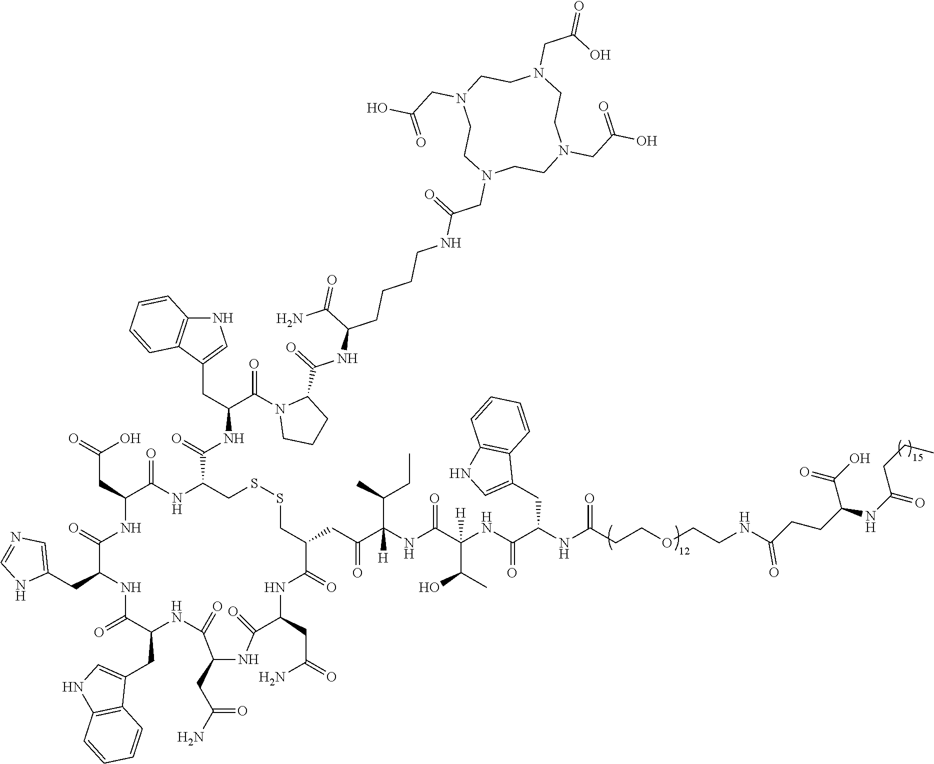

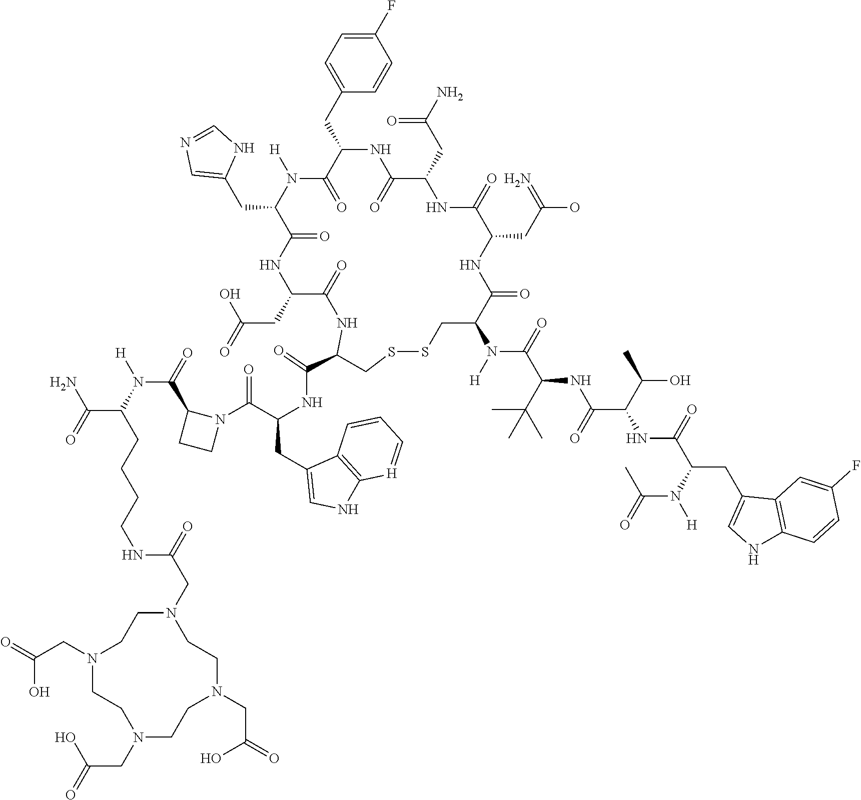

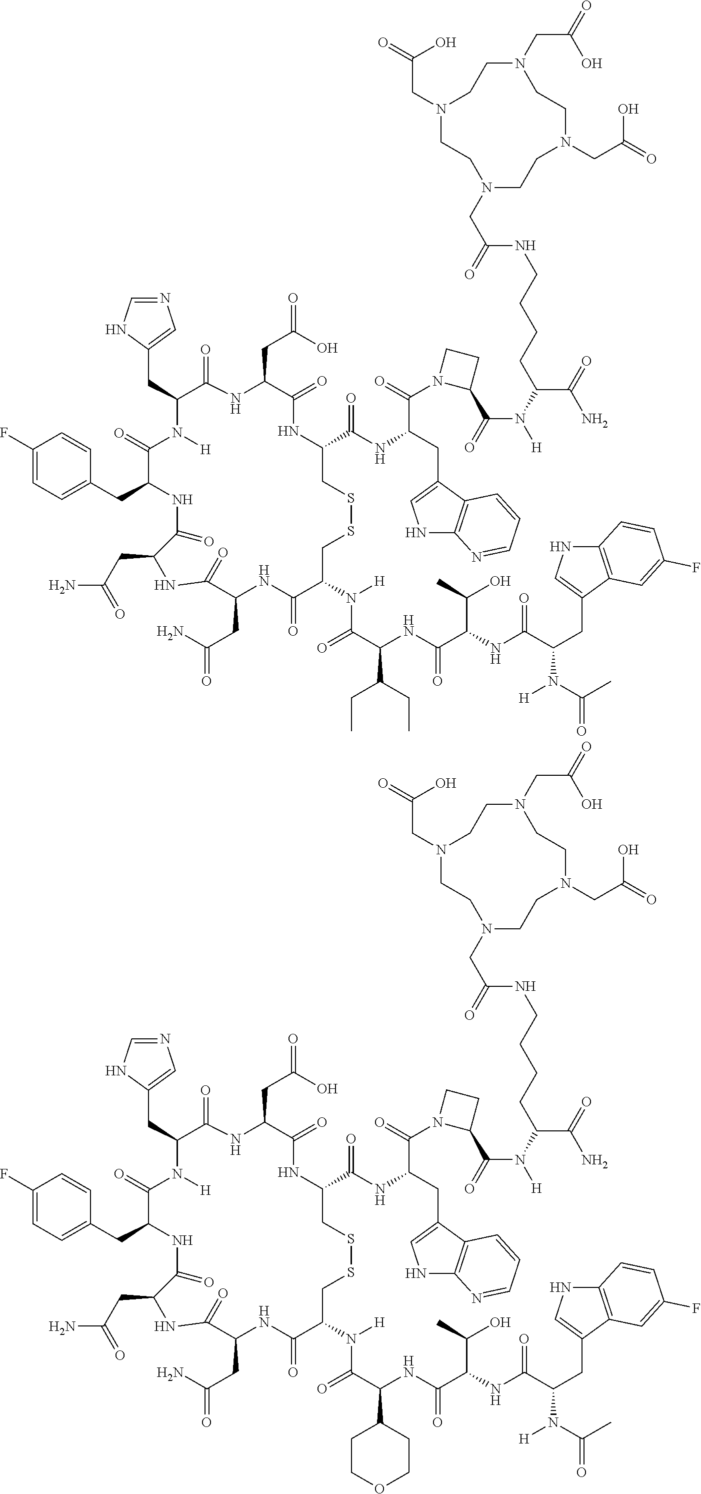

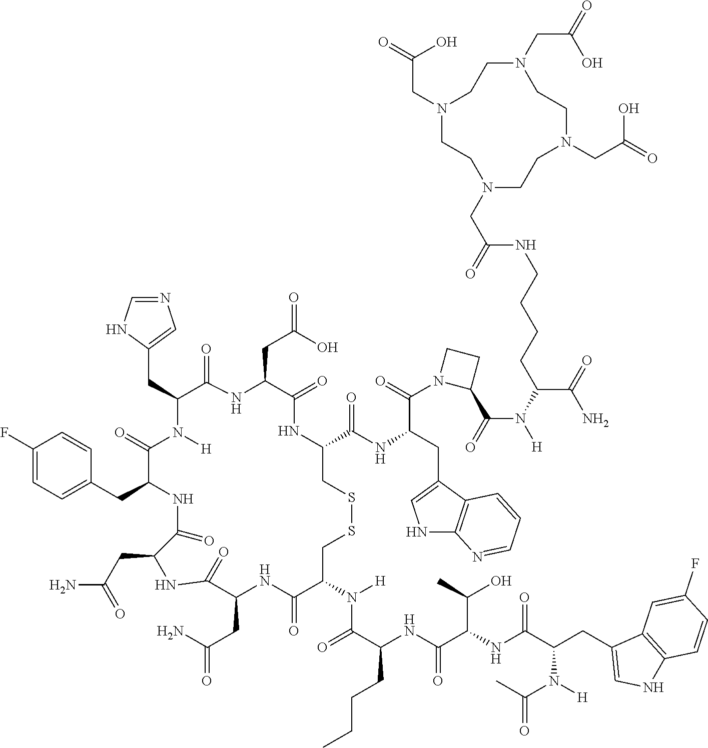

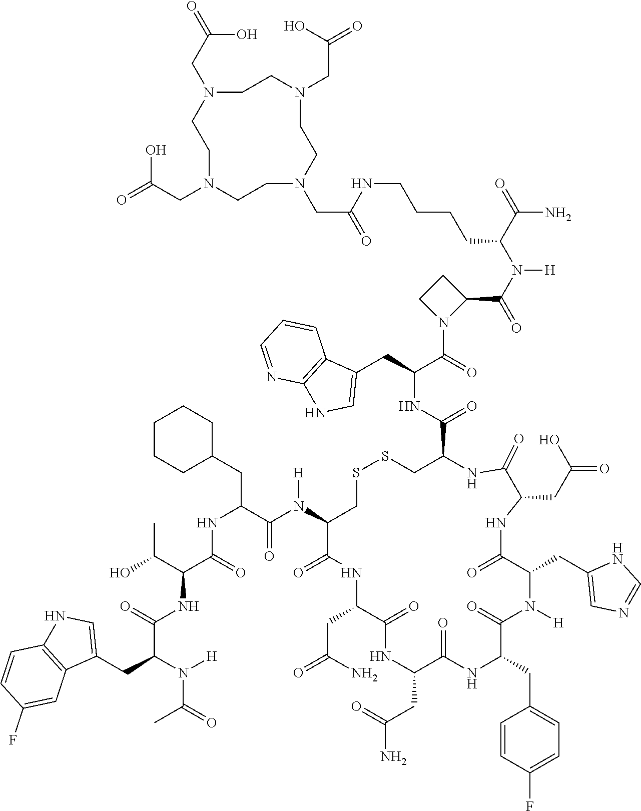 [SMILES: CC(=O)N[C@@H](Cc1c[nH]c2ccc(F)cc12)C(=O)N[C@H](C(=O)NC(CC1CCCCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O]

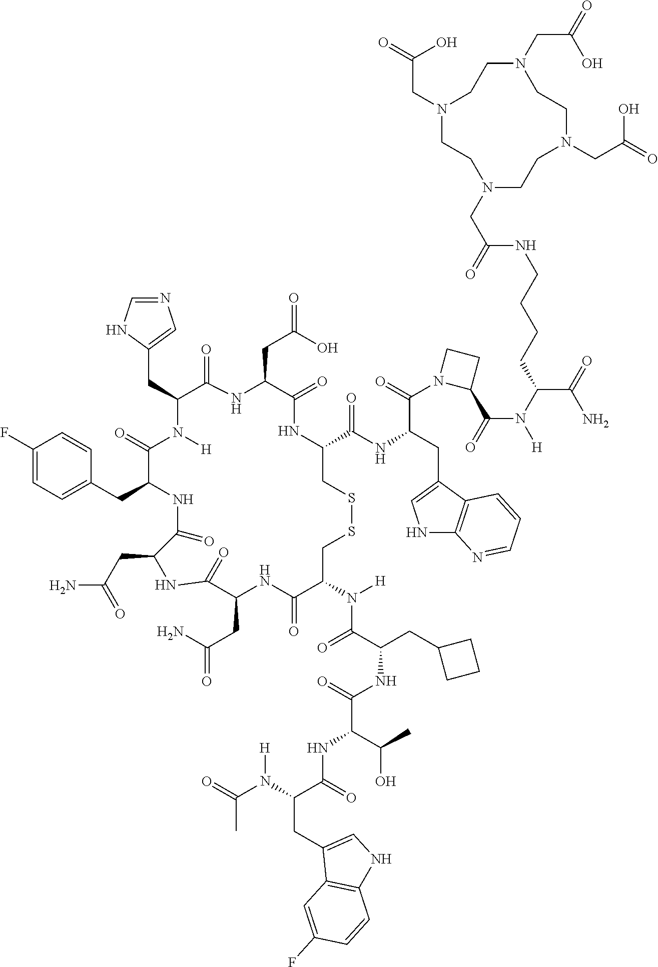 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O]

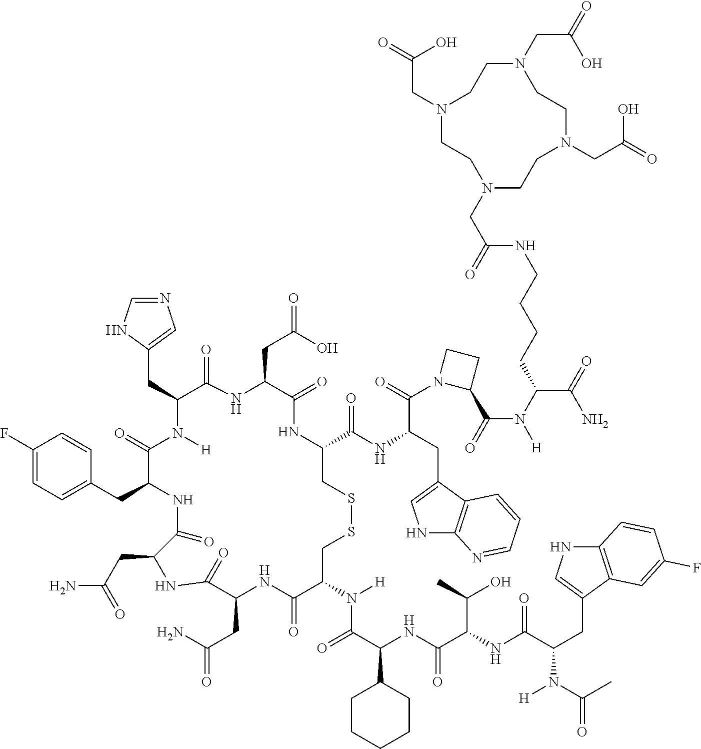 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)C1CCCCC1)[C@@H](C)O]

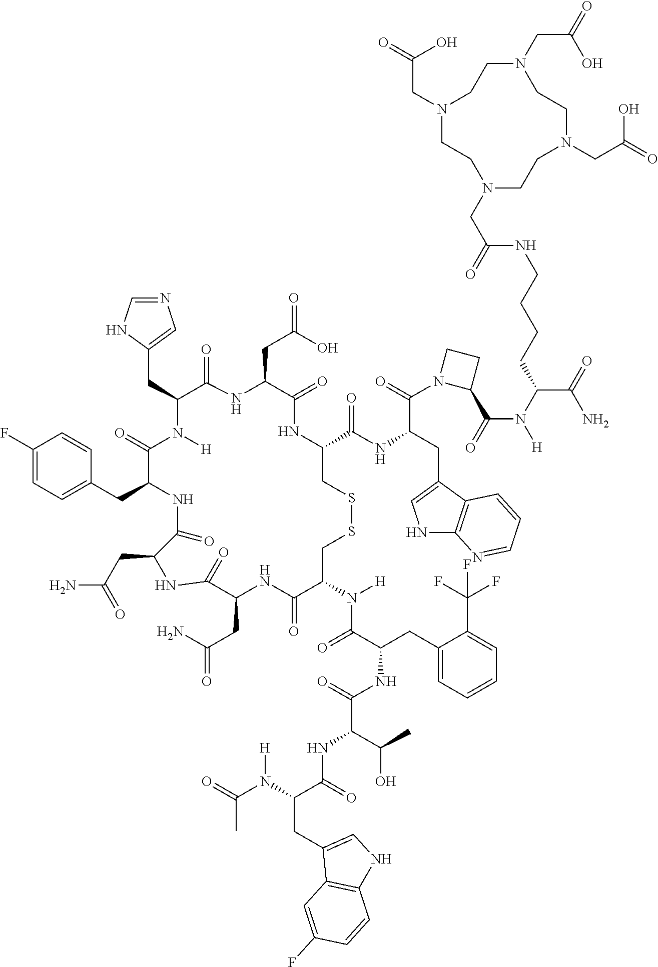 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O]

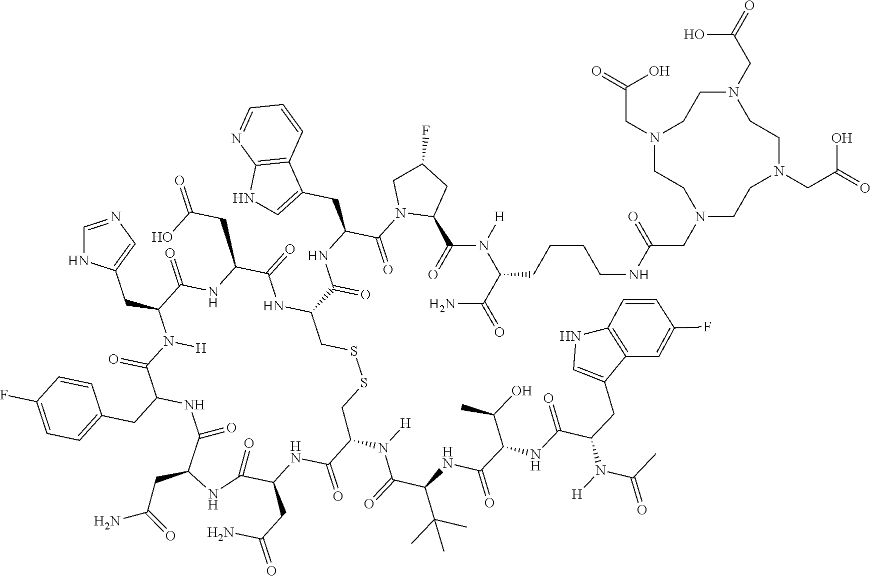 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)C(Cc2ccc(F)cc2)NC(=O)[C@H](CC(N)=O)NC(=O)[C@H](CC(N)=O)NC1=O)C(C)(C)C)[C@@H](C)O]

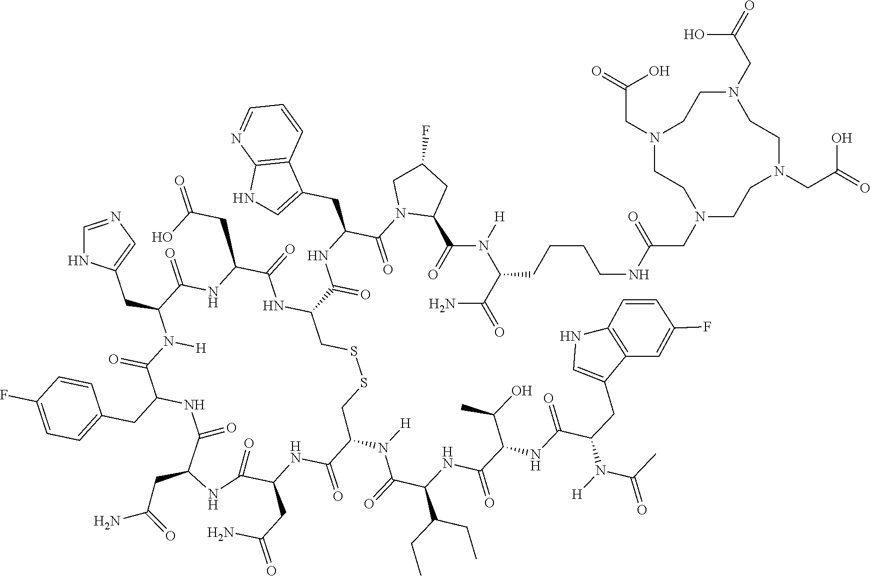 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)C(Cc2ccc(F)cc2)NC(=O)[C@H](CC(N)=O)NC(=O)[C@H](CC(N)=O)NC1=O]

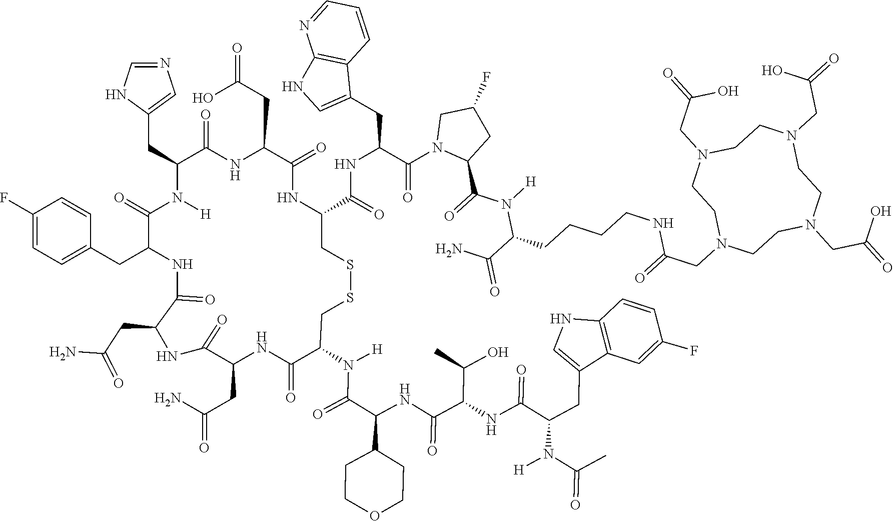 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)C(Cc2ccc(F)cc2)NC(=O)[C@H](CC(N)=O)NC(=O)[C@H](CC(N)=O)NC1=O)C1CCOCC1)[C@@H](C)O]

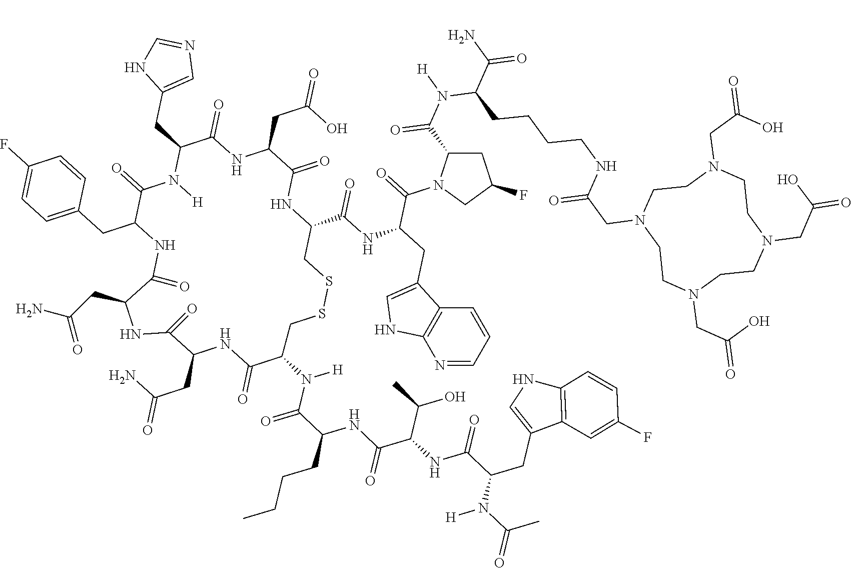

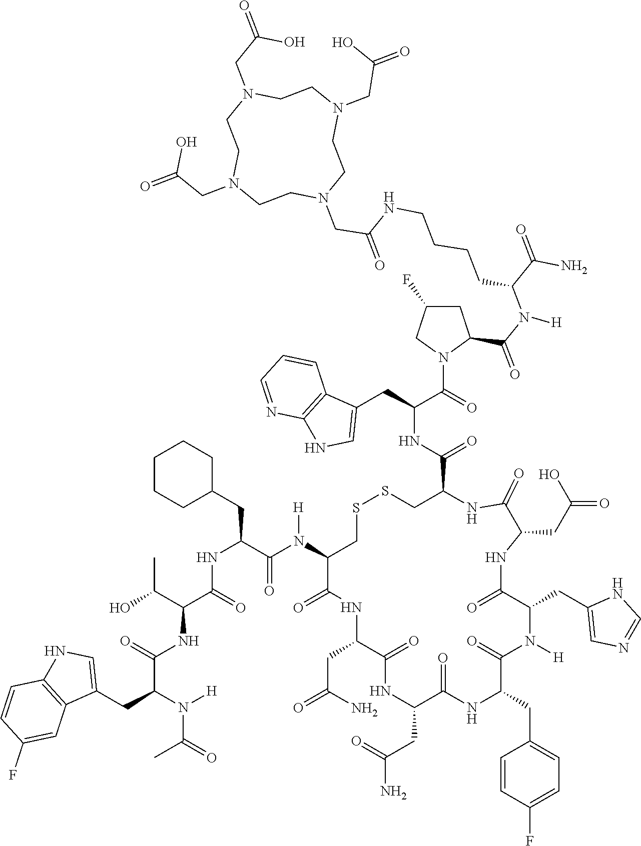 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O]

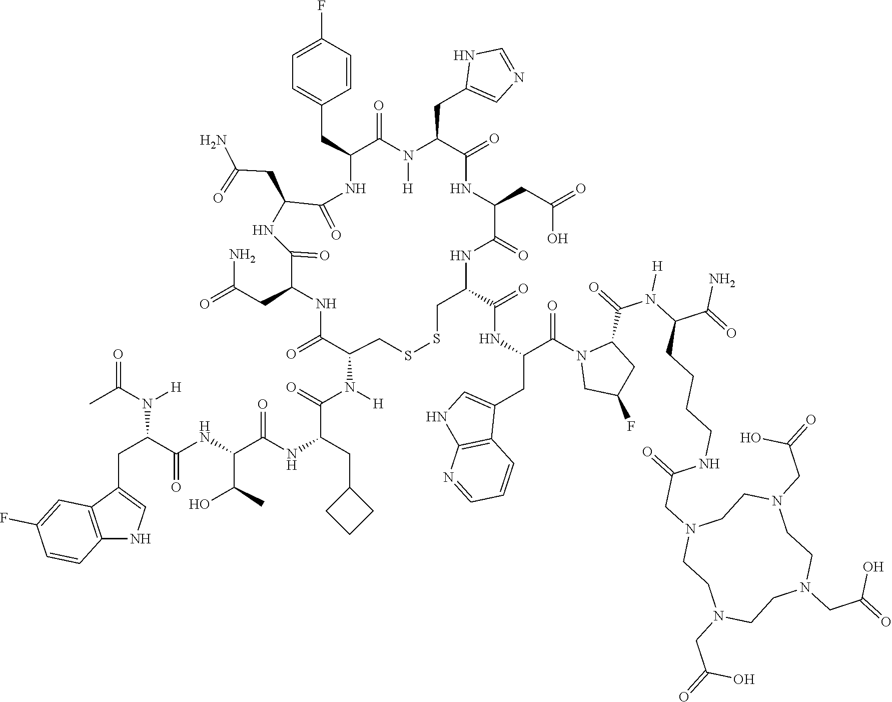

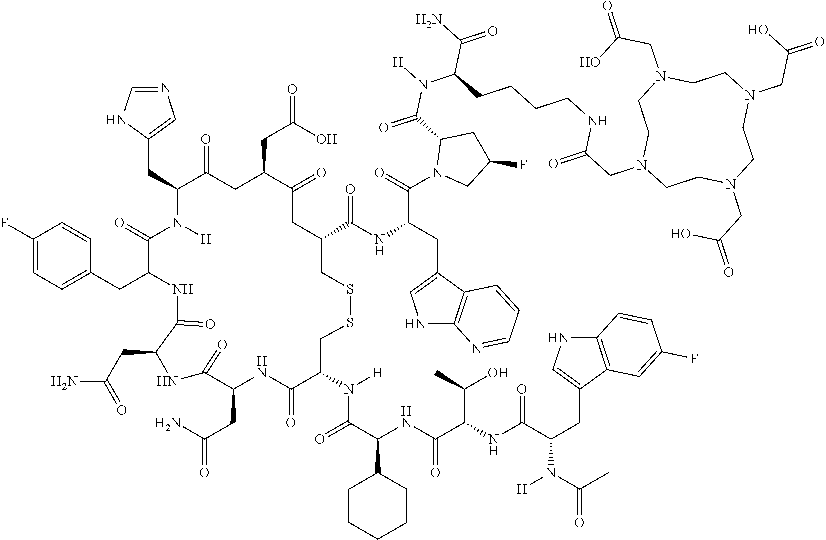 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)CC(=O)[C@H](CC(=O)O)CC(=O)[C@H](Cc2cnc[nH]2)NC(=O)C(Cc2ccc(F)cc2)NC(=O)[C@H](CC(N)=O)NC(=O)[C@H](CC(N)=O)NC1=O)C1CCCCC1)[C@@H](C)O]

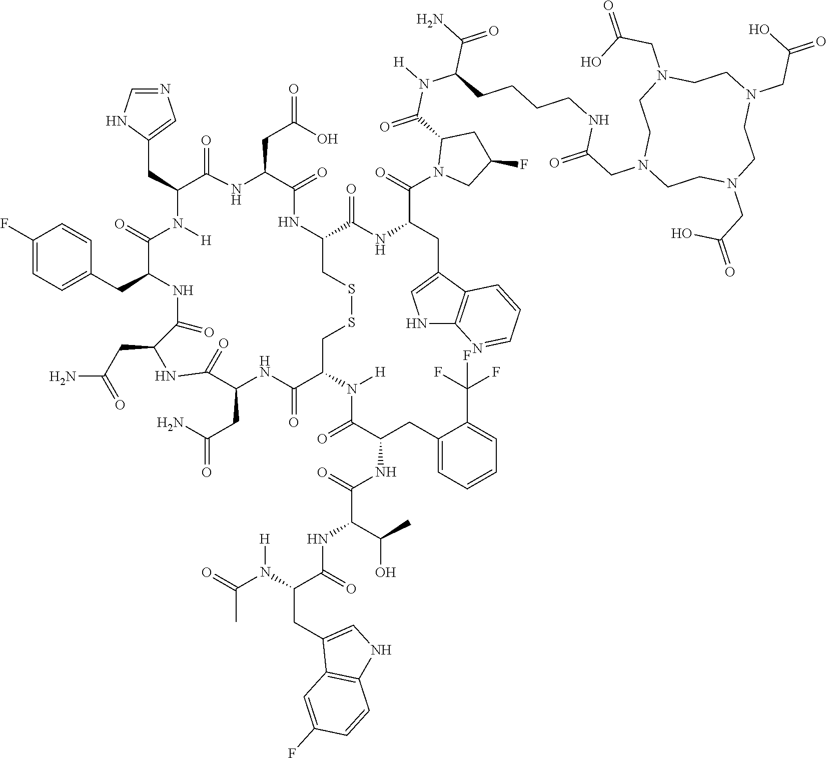 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(F)cc2)NC(=O)[C@H](CC(N)=O)NC(=O)[C@H](CC(N)=O)NC1=O)[C@@H](C)O]

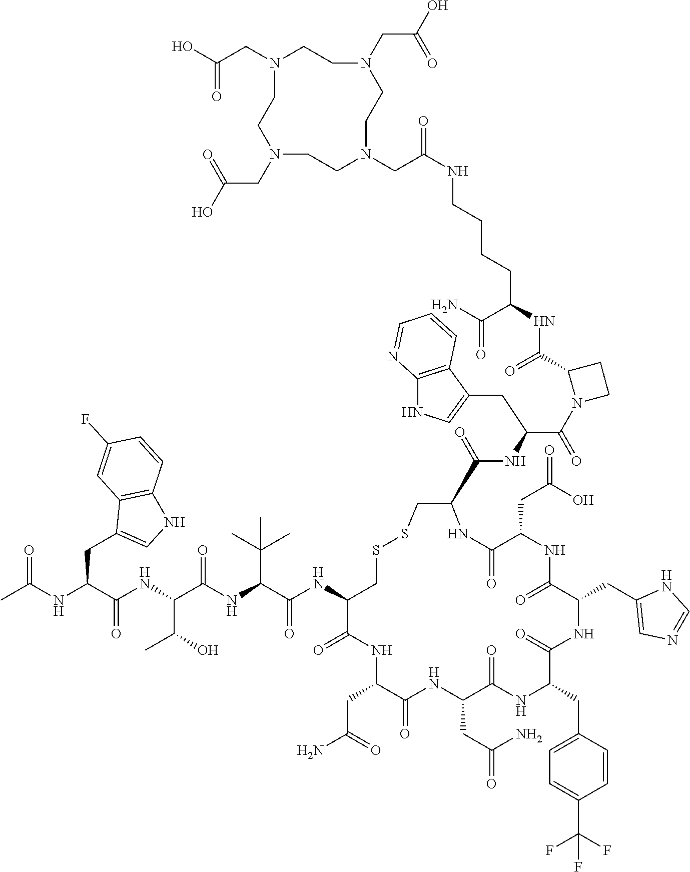 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C(C)(C)C)[C@@H](C)O]

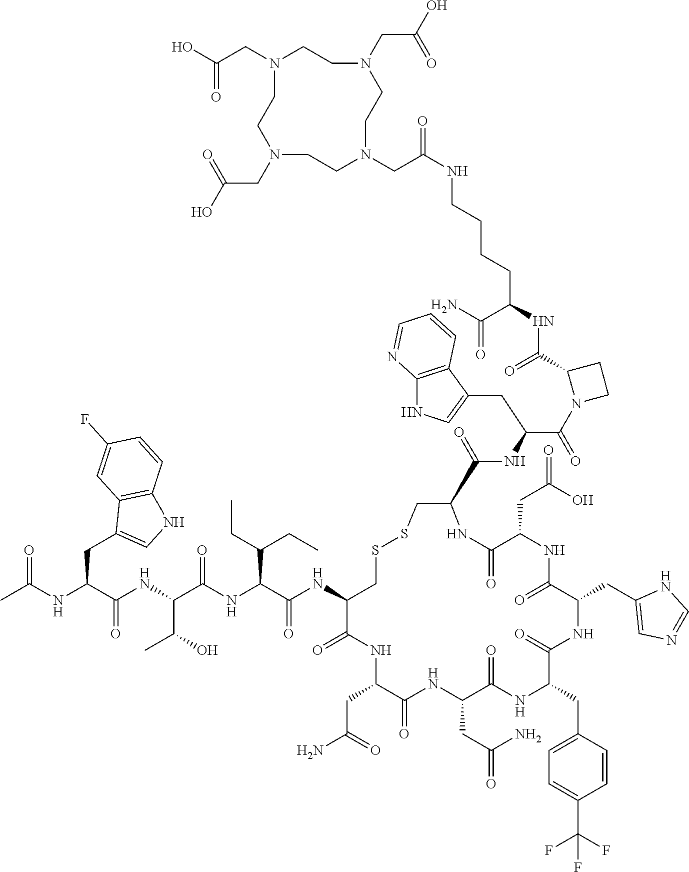 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O]

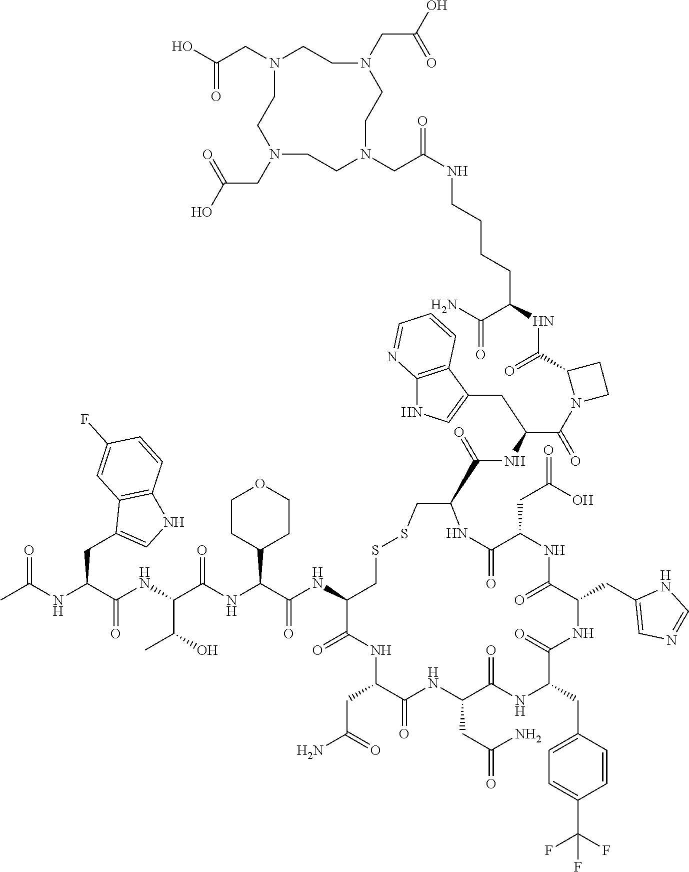 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C1CCOCC1)[C@@H](C)O]

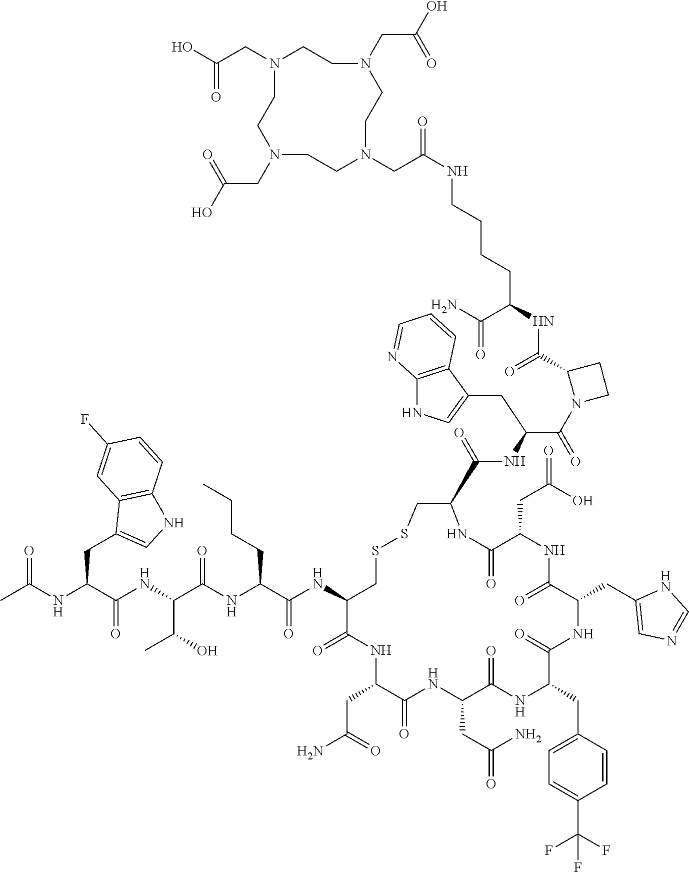 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O]

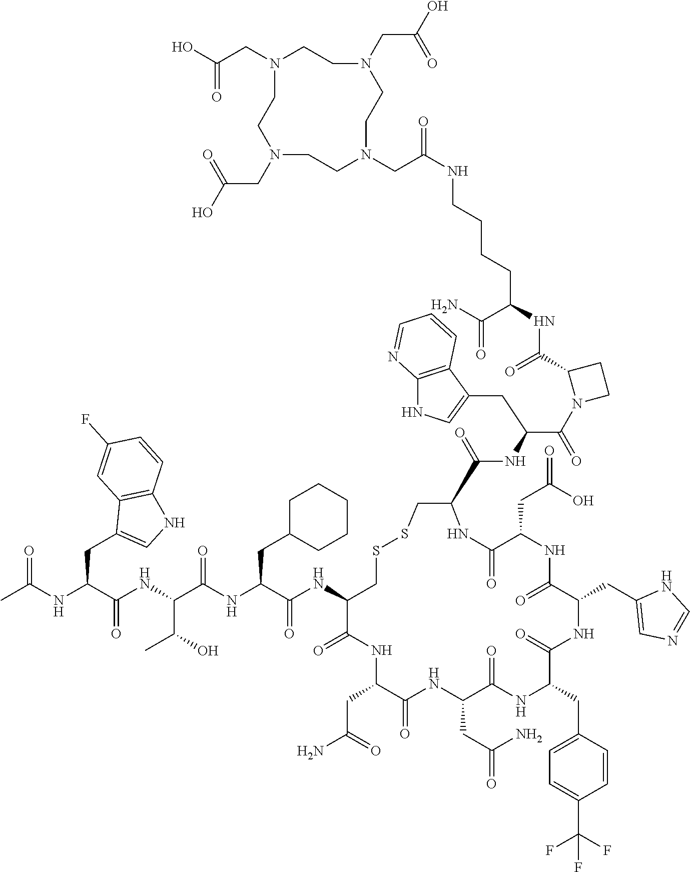 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O]

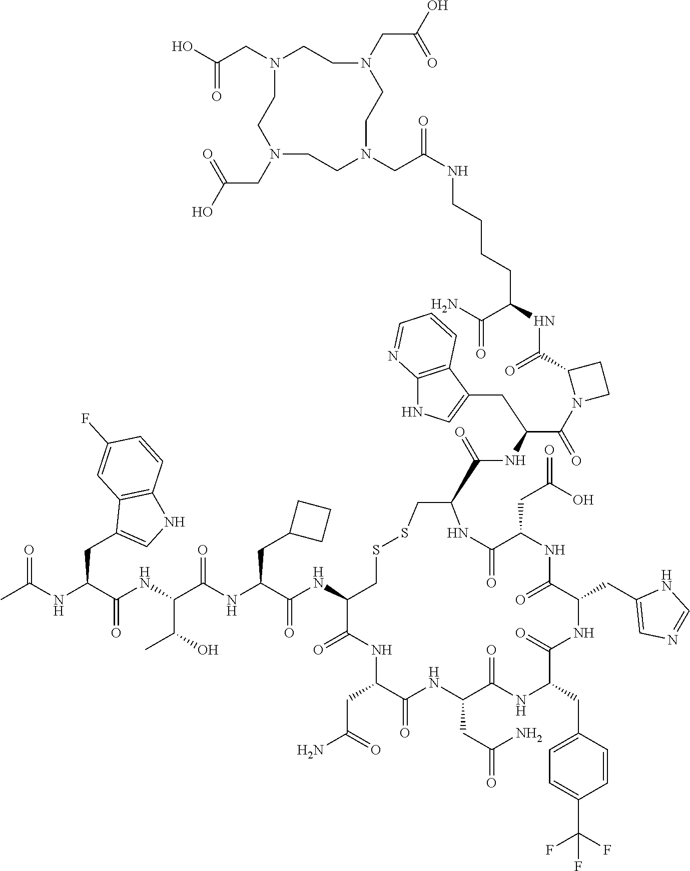 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O]

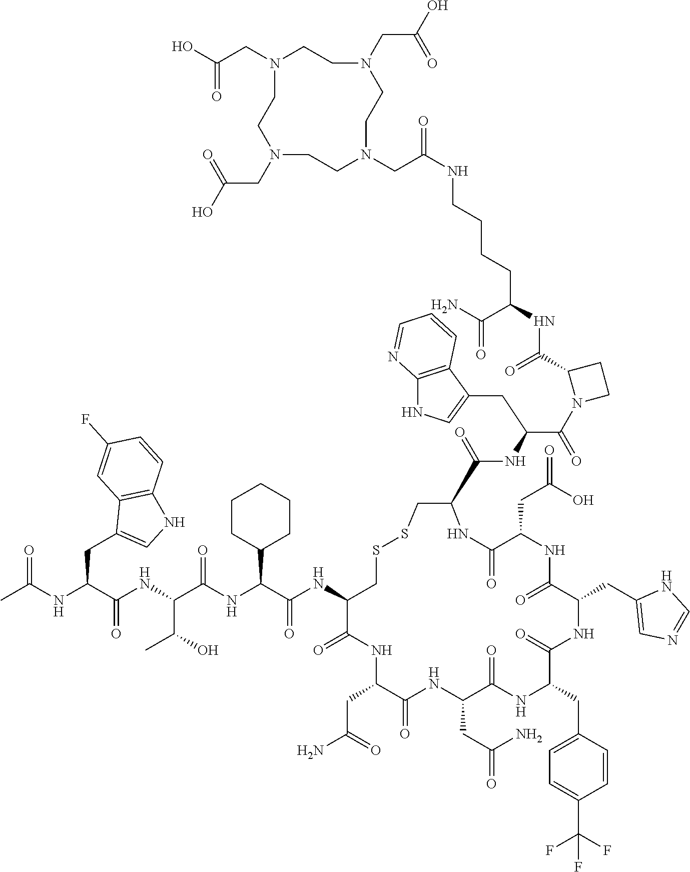 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C1CCCCC1)[C@@H](C)O]

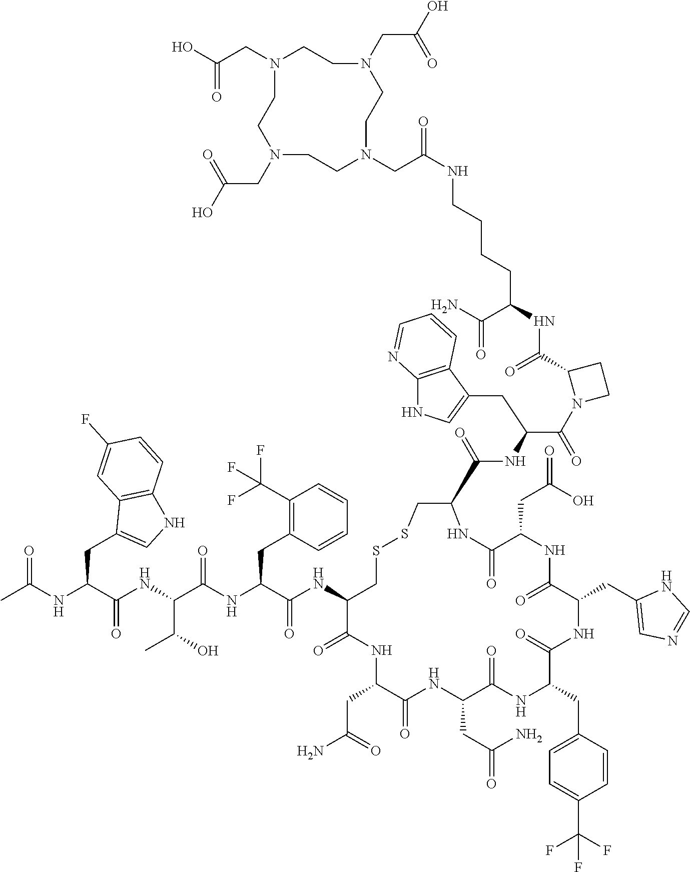 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2CC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc(C(F)(F)F)cc2)NC(=O)[C@H](CC(N)=O)NC(=O)[C@H](CC(N)=O)NC1=O)[C@@H](C)O]

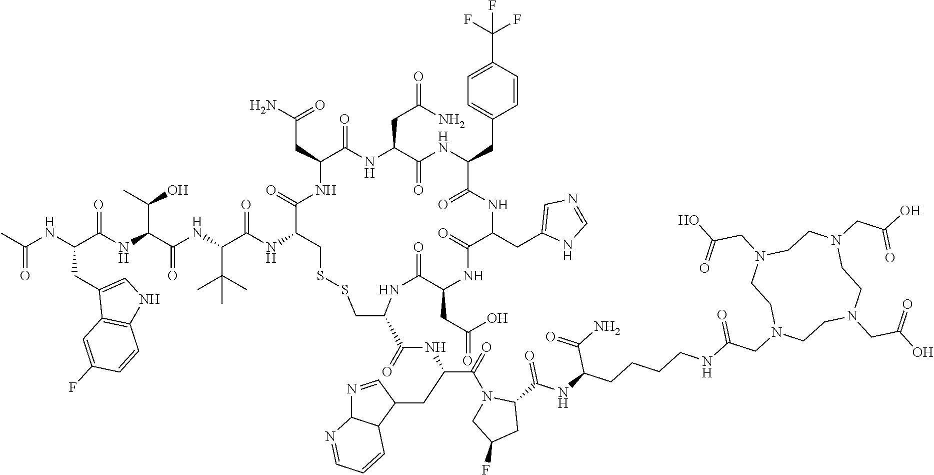 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](CC2C=NC3N=CC=CC23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)C(Cc2cnc[nH]2)NC(=O)[C@H](Cc2ccc(C(F)(F)F)cc2)NC(=O)[C@H](CC(N)=O)NC(=O)[C@H](CC(N)=O)NC1=O)C(C)(C)C)[C@@H](C)O]

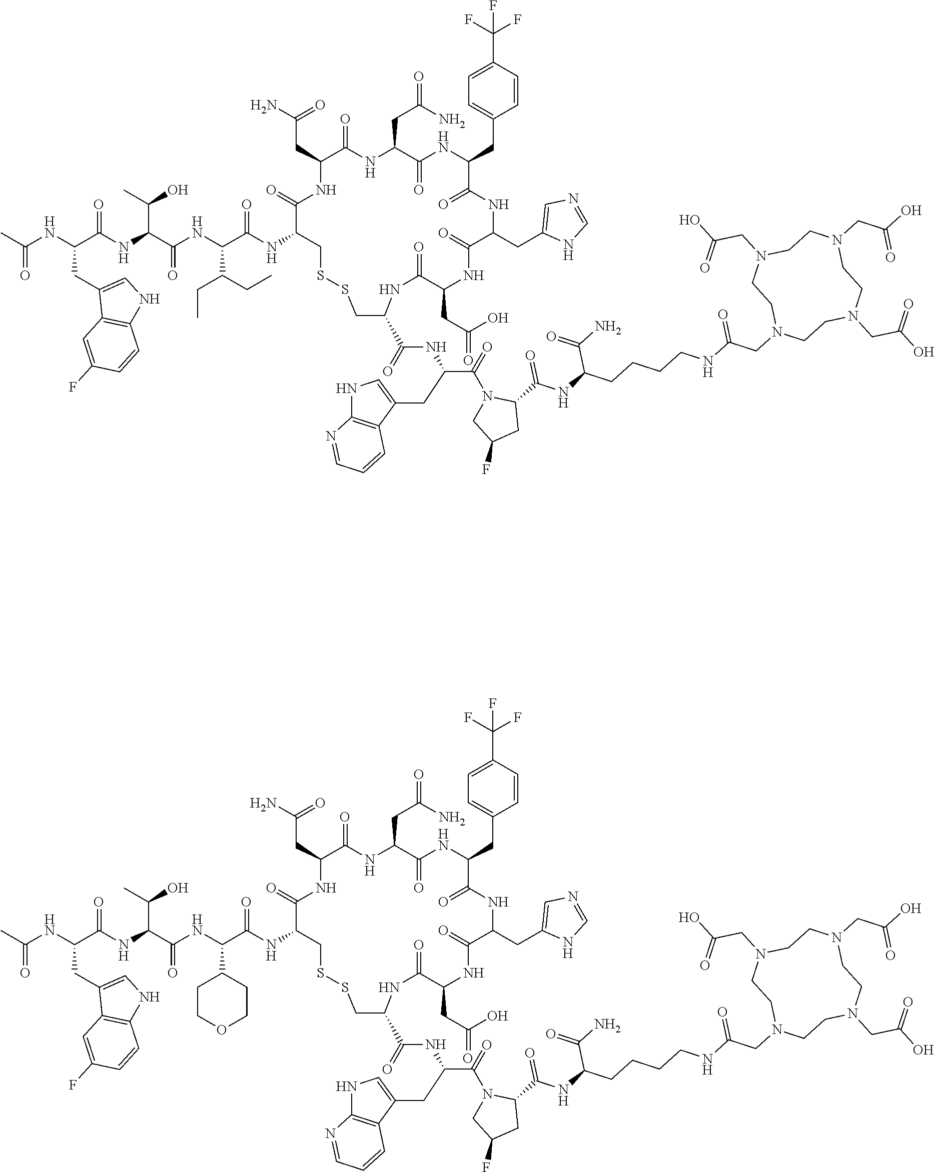

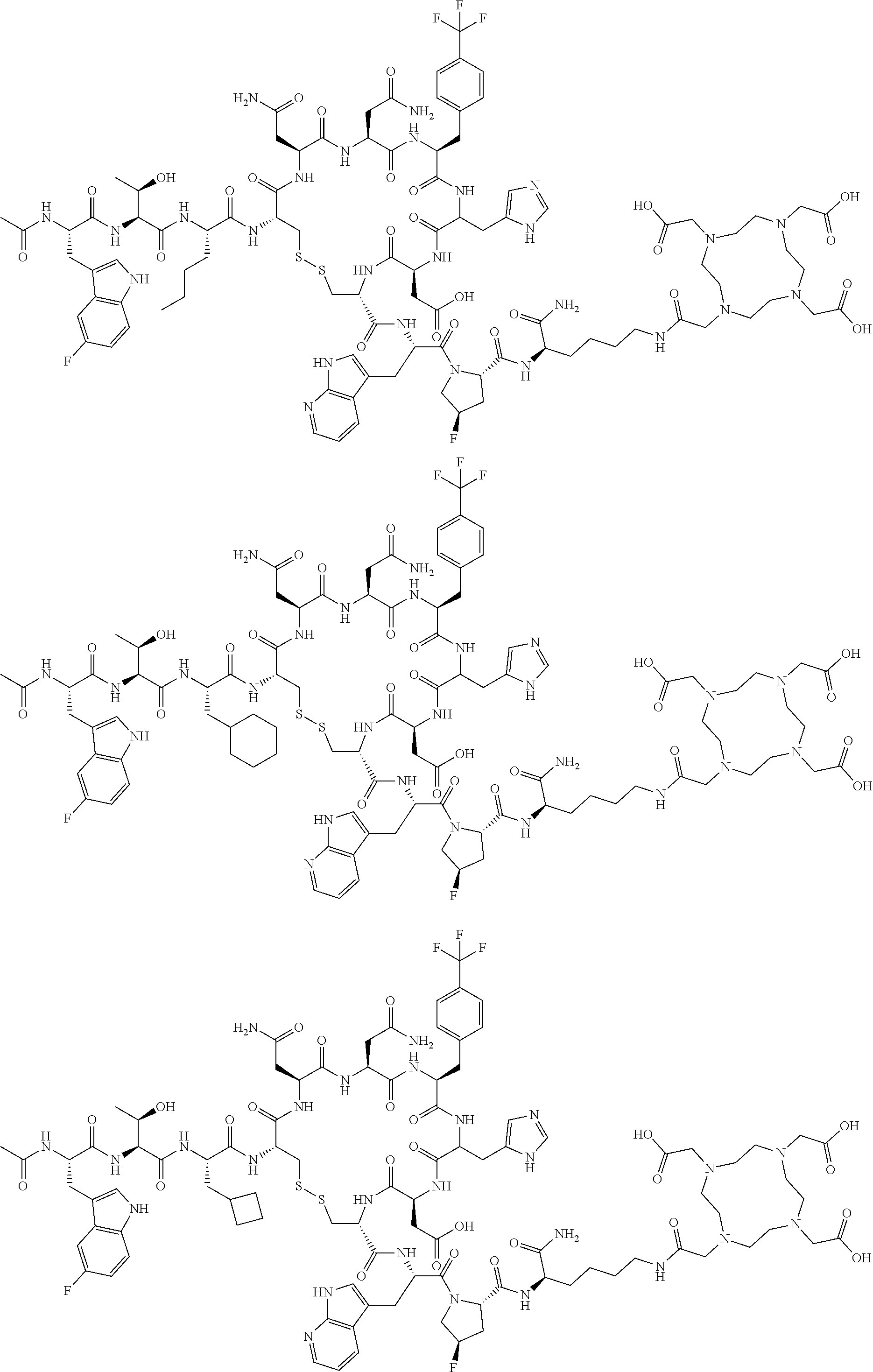

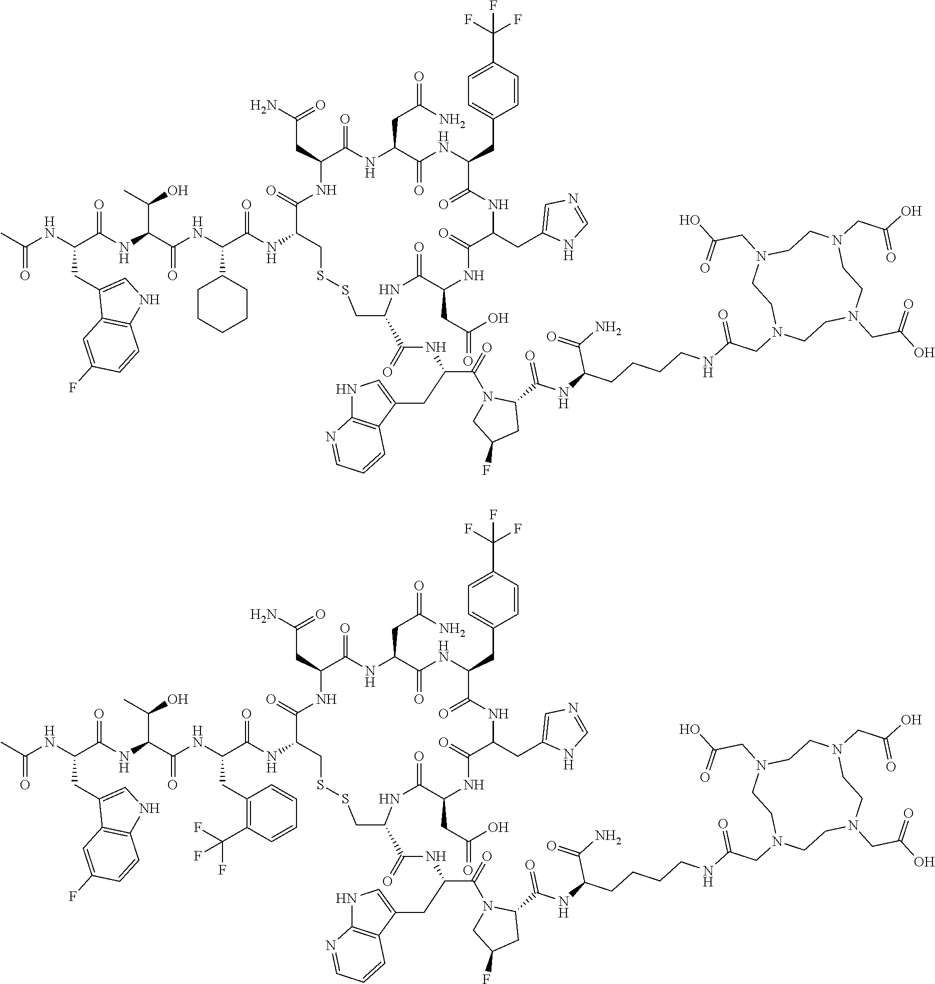

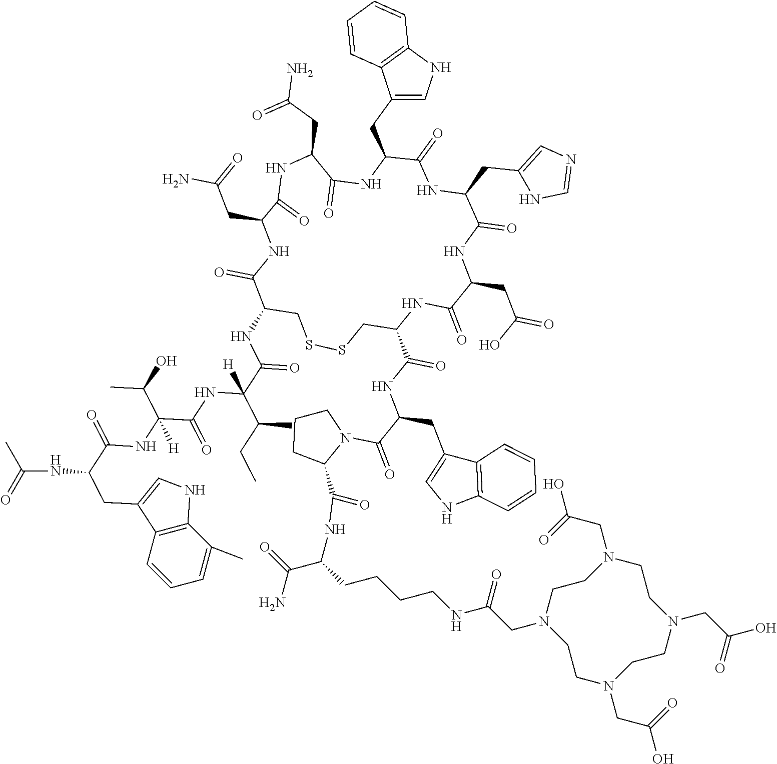 [SMILES: CC[C@H](C)[C@H](NC(=O)[C@@H](NC(=O)[C@H](Cc1c[nH]c2c(C)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

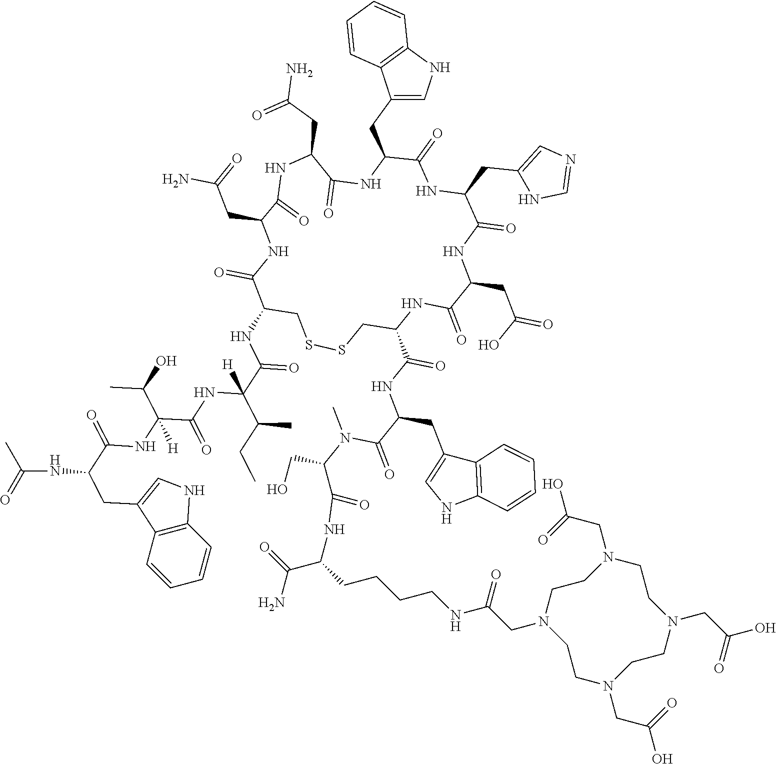 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)[C@@H](CO)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

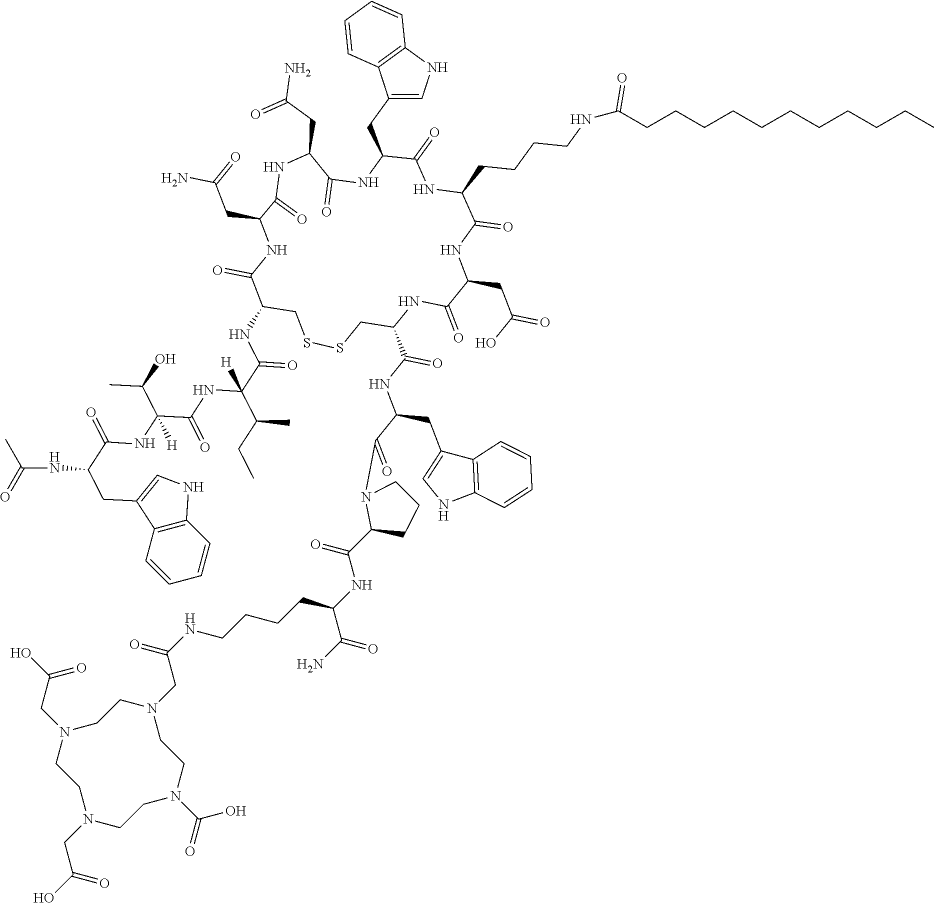 [SMILES: CCCCCCCCCCCC(=O)NCCCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(C(=O)O)CC2)C(N)=O)NC(=O)[C@H](CC(=O)O)NC1=O]

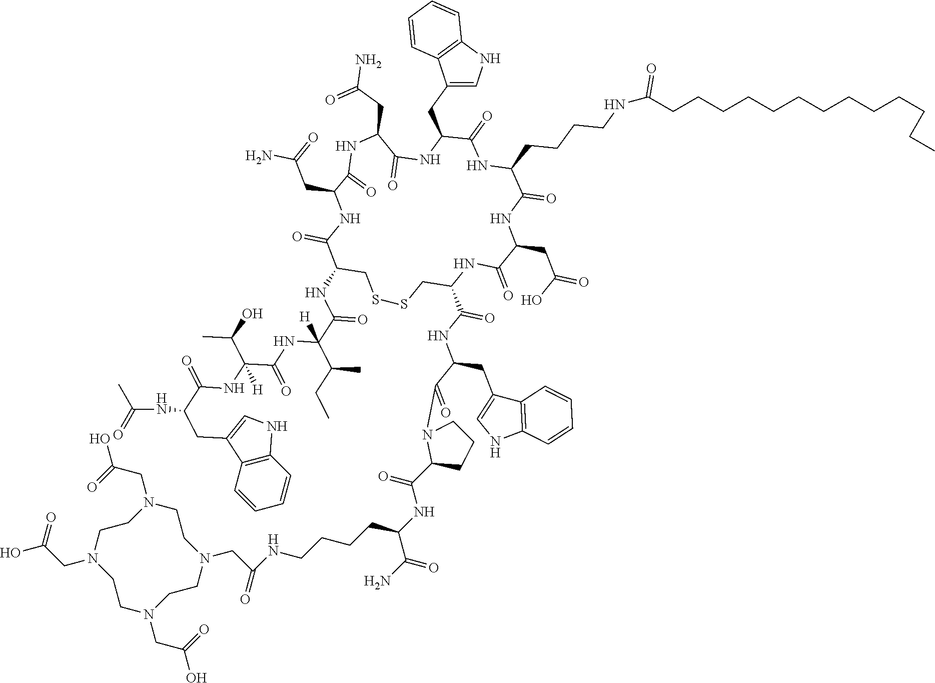 [SMILES: CCCCCCCCCCCCCC(=O)NCCCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O]

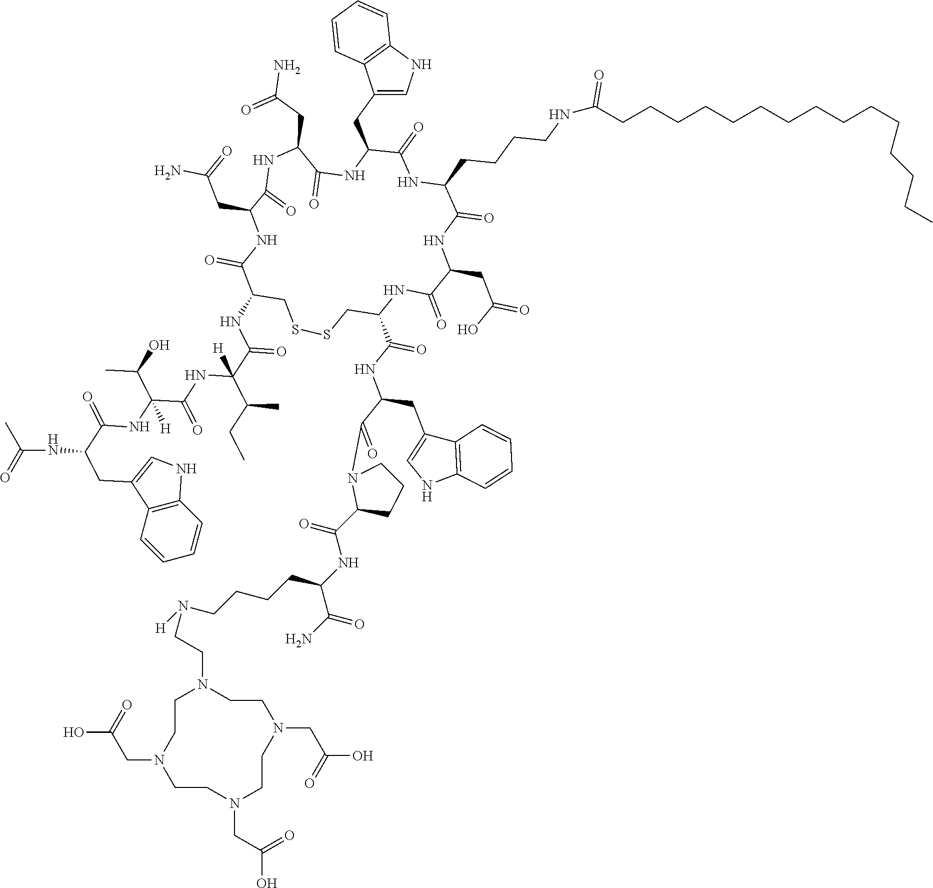 [SMILES: CCCCCCCCCCCCCCCC(=O)NCCCC[C@@H]1NC(=O)[C@H](Cc2c[nH]c3ccccc23)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNCCN2CCN(CC(=O)O)CCN(CC(=O)O)CCN(CC(=O)O)CC2)C(N)=O)NC(=O)[C@H](CC(=O)O)NC1=O]

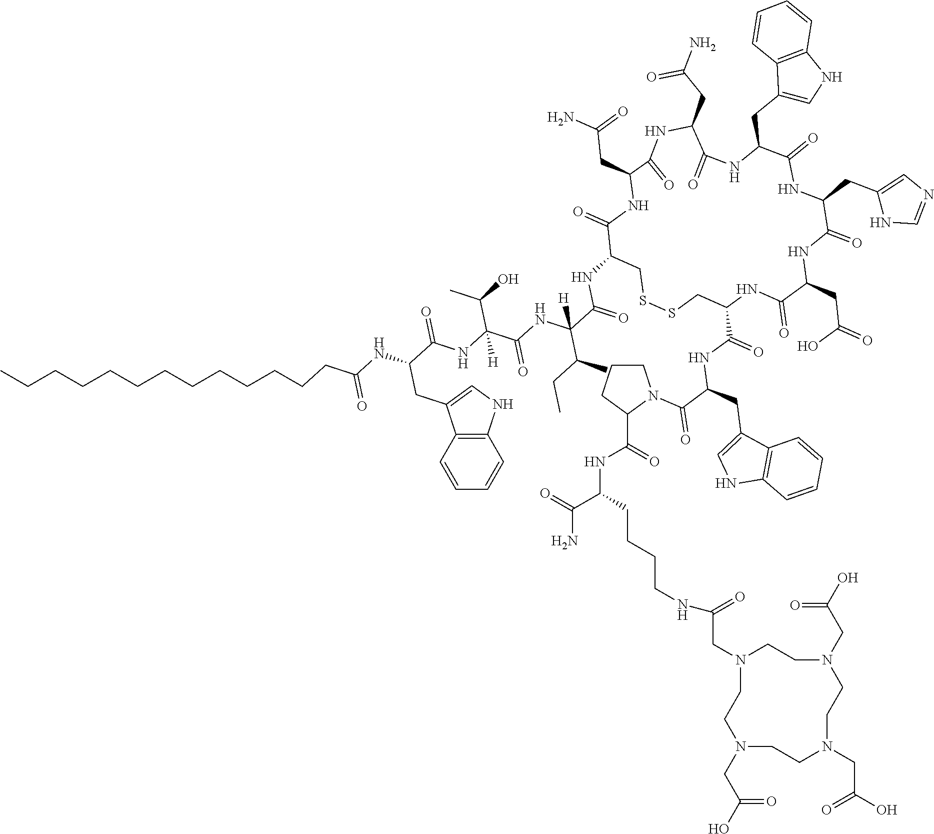 [SMILES: CCCCCCCCCCCCCC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCCC2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O)[C@@H](C)CC)[C@@H](C)O]

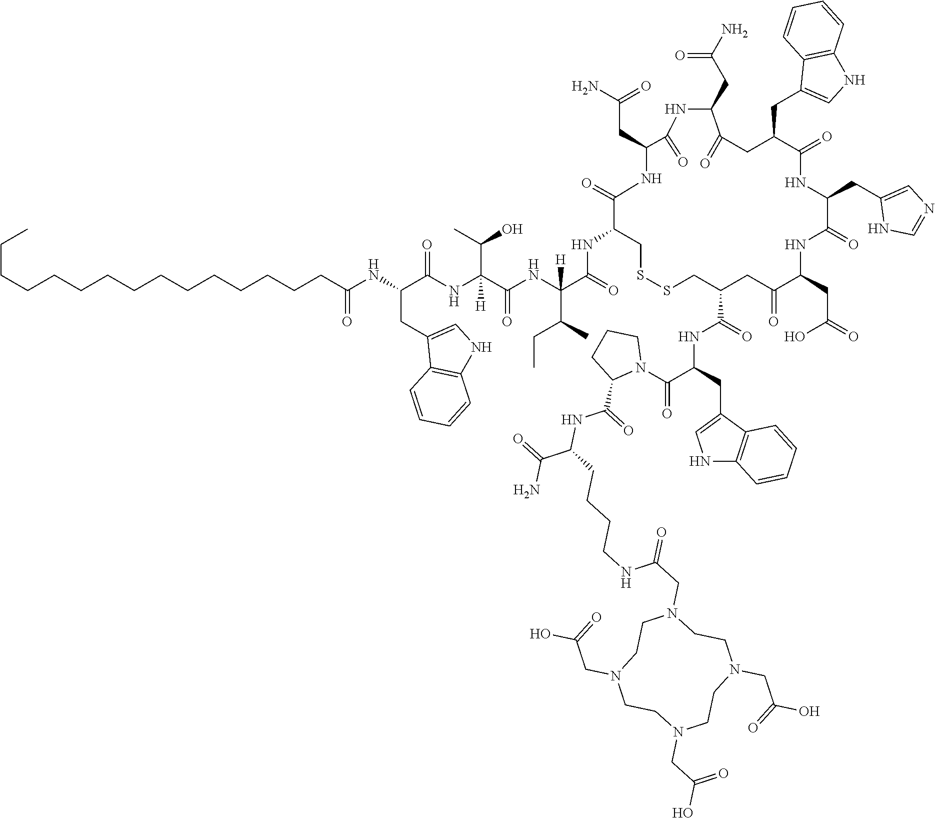 [SMILES: CCCCCCCCCCCCCCCC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)CC(=O)[C@H](CC(=O)O)NC(=O)[C@H](Cc2cnc[nH]2)NC(=O)[C@H](Cc2c[nH]c3ccccc23)CC(=O)[C@H](CC(N)=O)NC(=O)[C@H](CC(N)=O)NC1=O)[C@@H](C)CC)[C@@H](C)O]

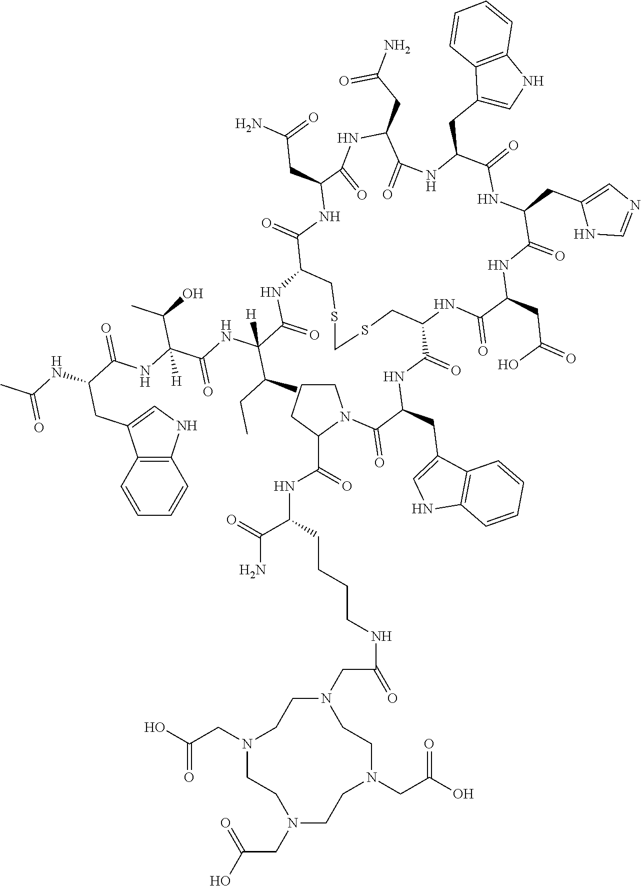 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCSC[C@@H](C(=O)N[C@@H](Cc2c[nH]c3ccccc23)C(=O)N2CCCC2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

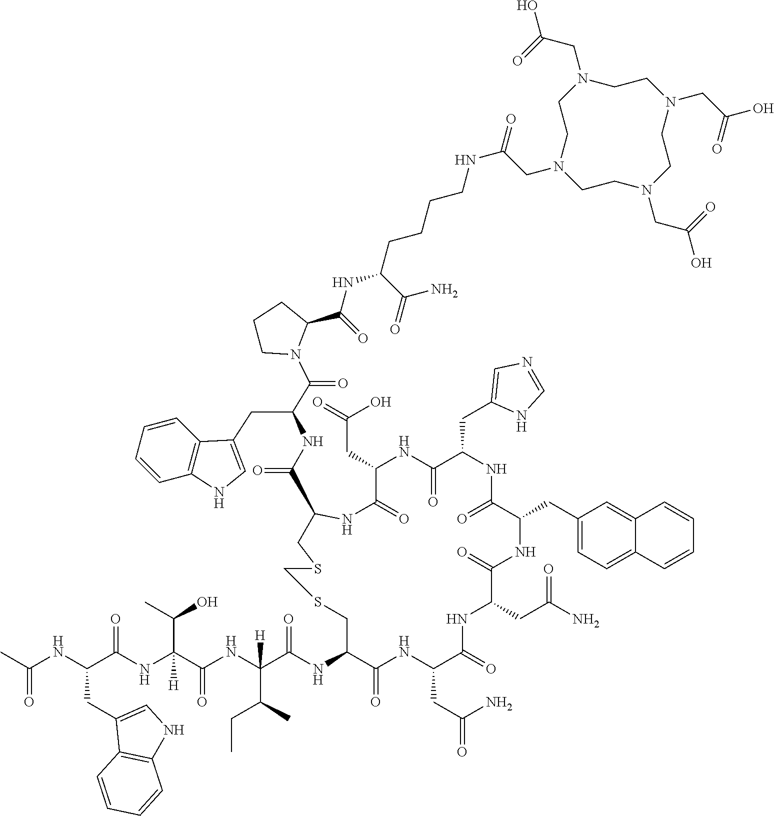 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

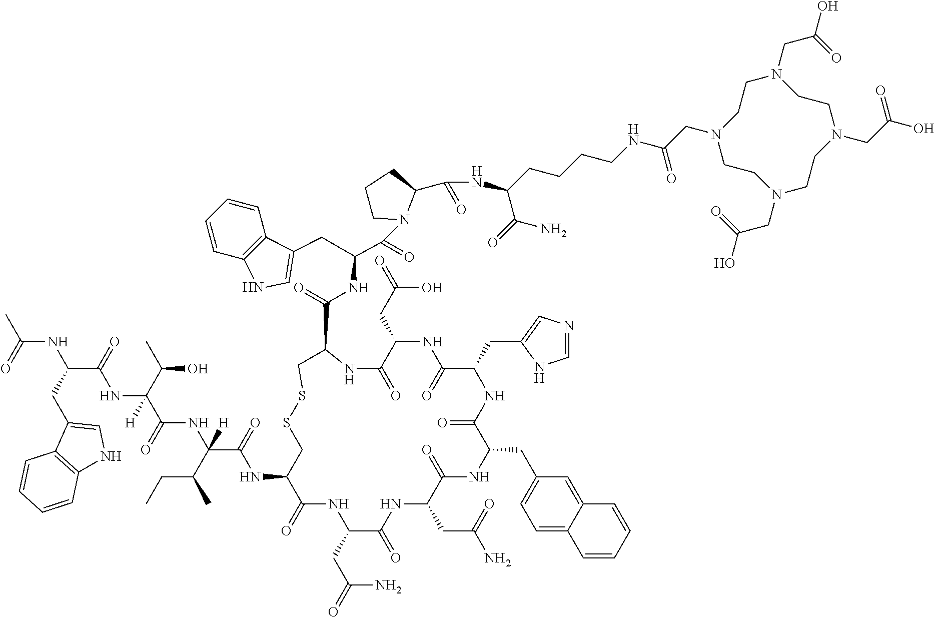 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

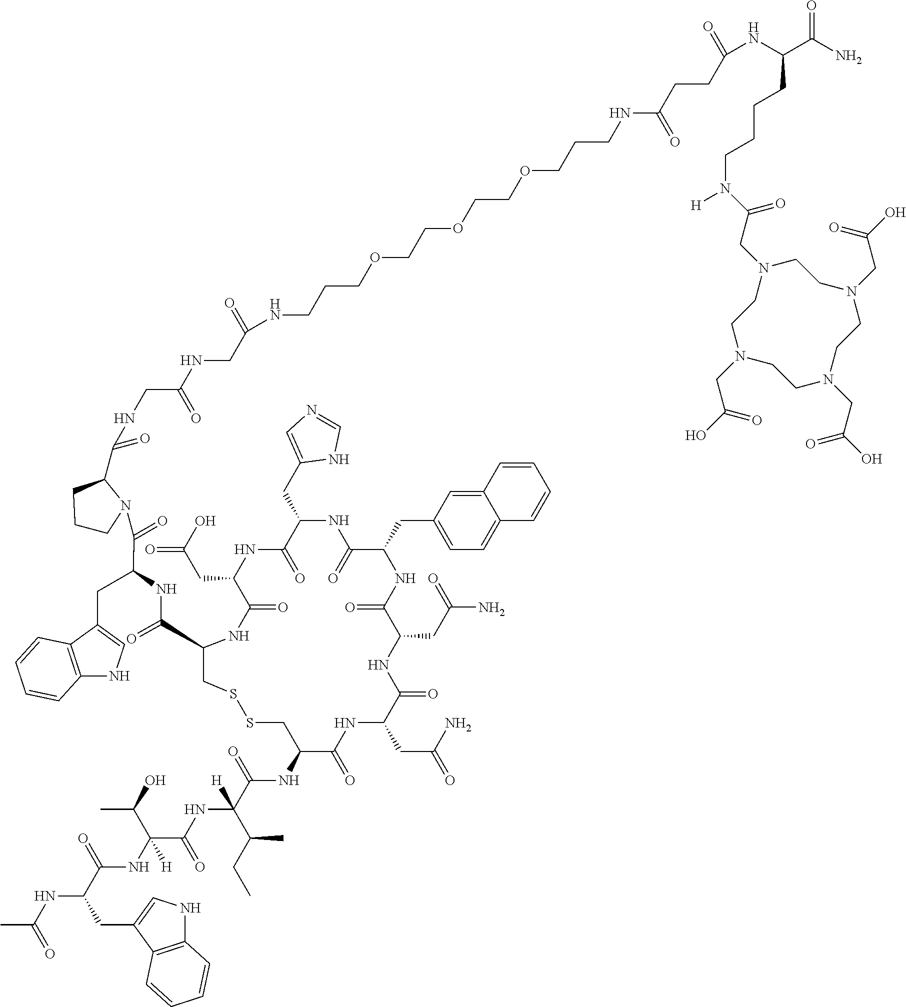 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCC(=O)NCC(=O)NCCCOCCOCCOCCCNC(=O)CCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

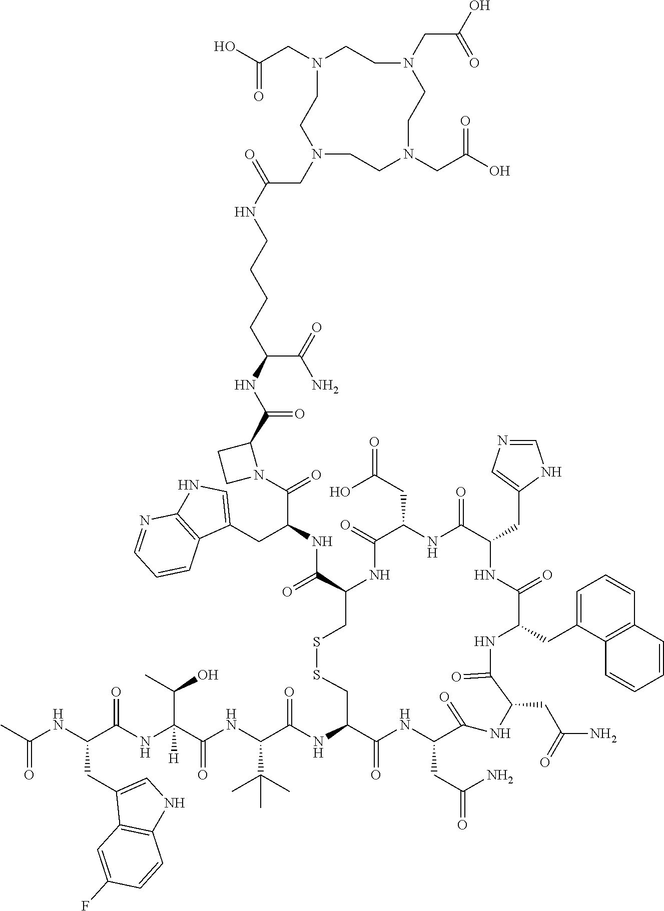 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C(C)(C)C)[C@@H](C)O]

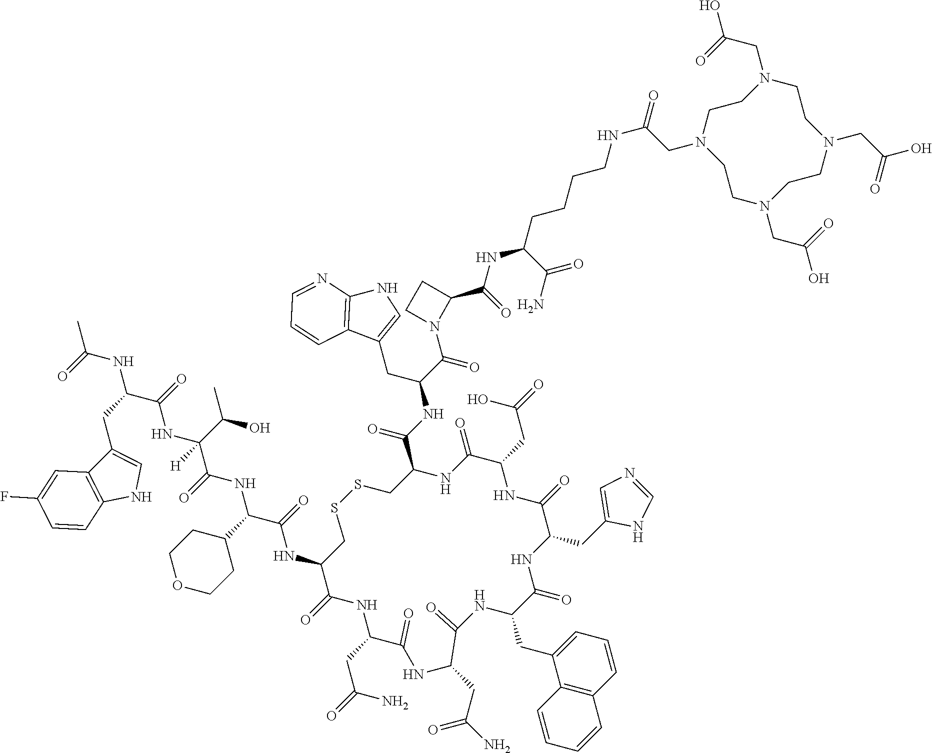 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C1CCOCC1)[C@@H](C)O]

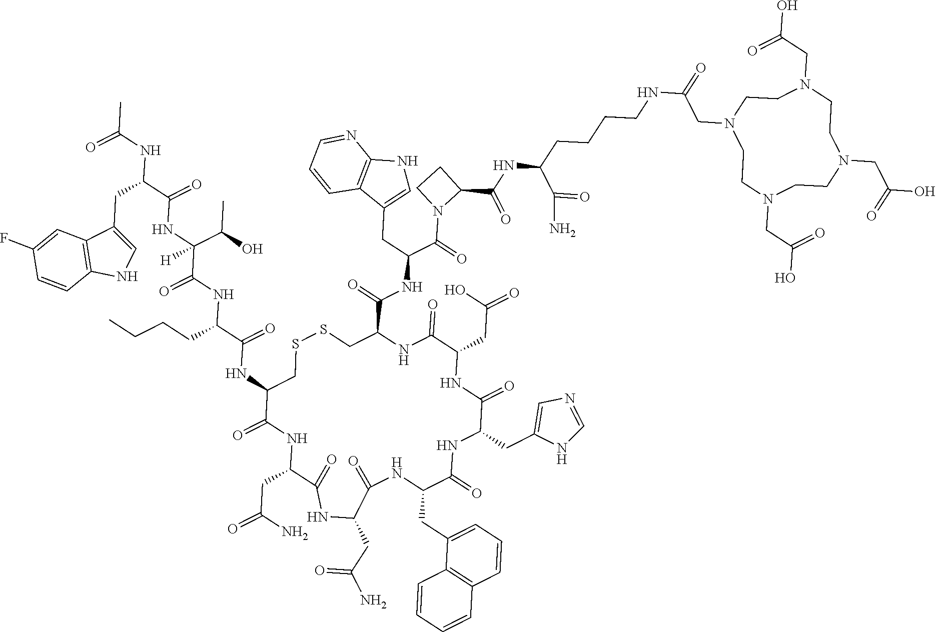 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O]

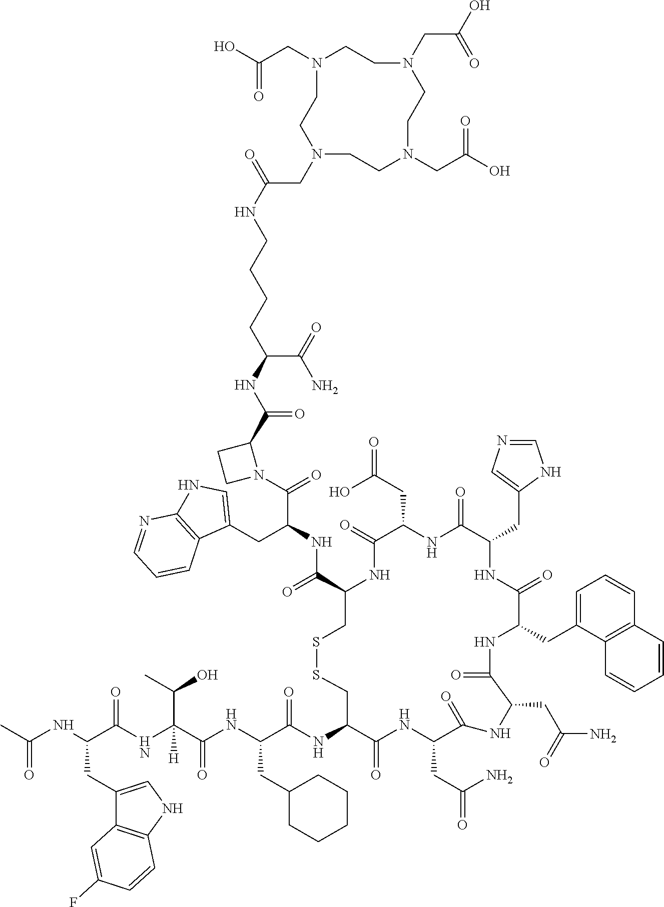 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O]

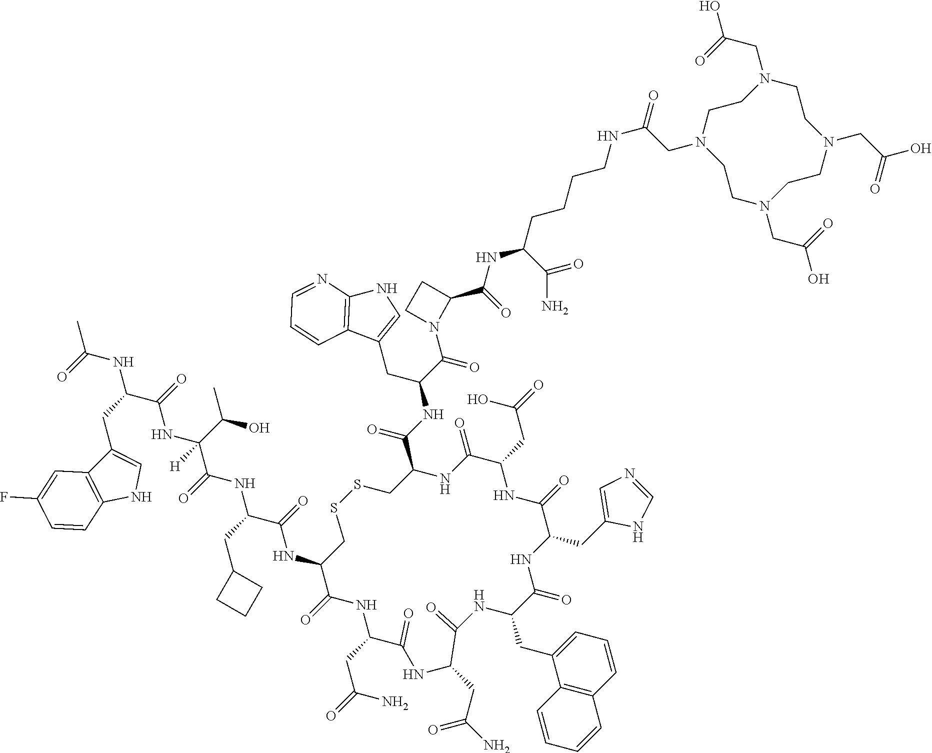 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O]

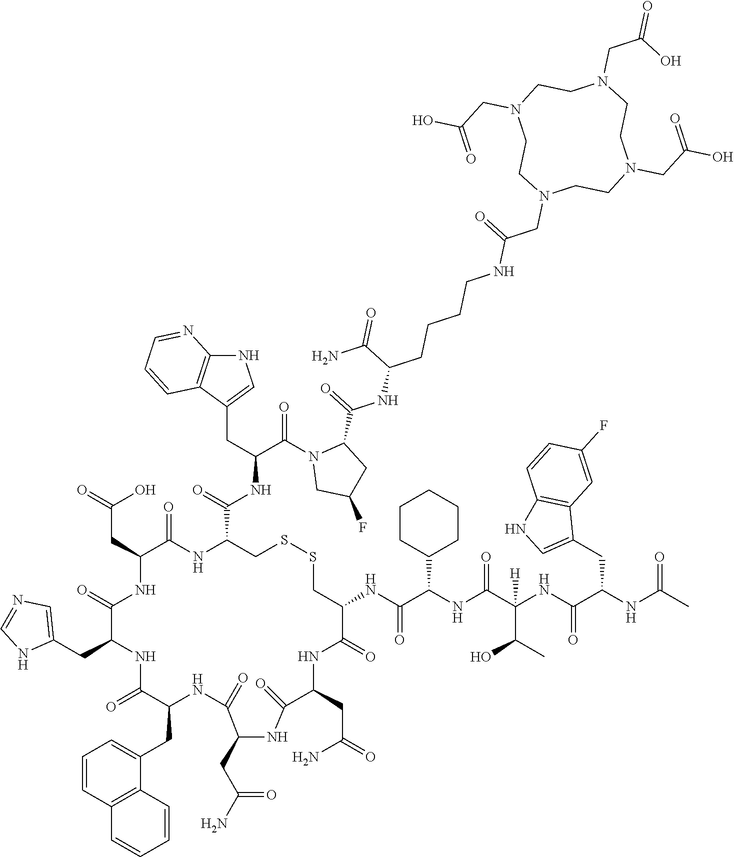 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C1CCCCC1)[C@@H](C)O]

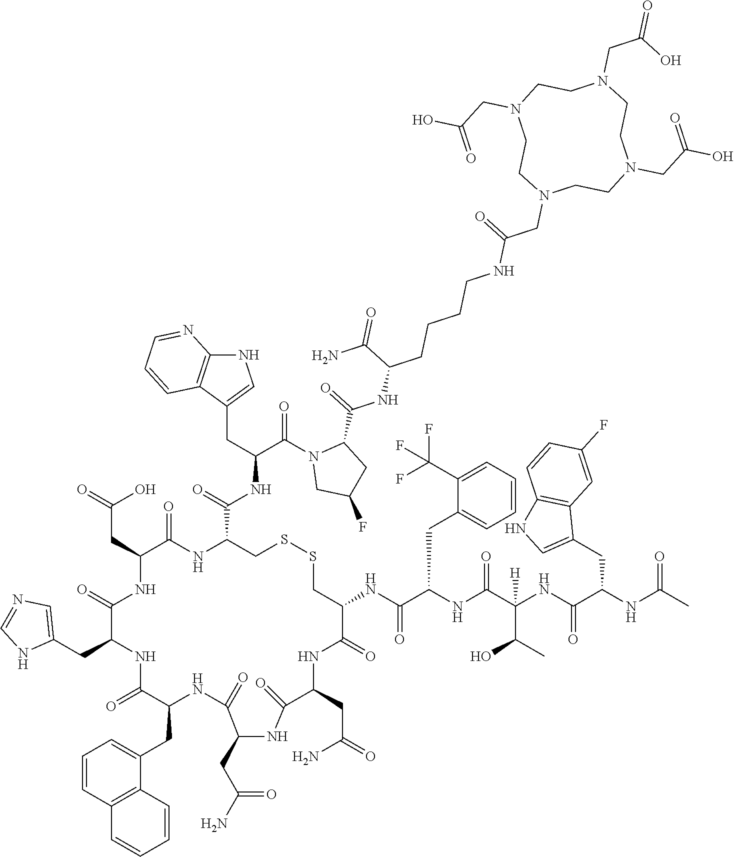 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O]

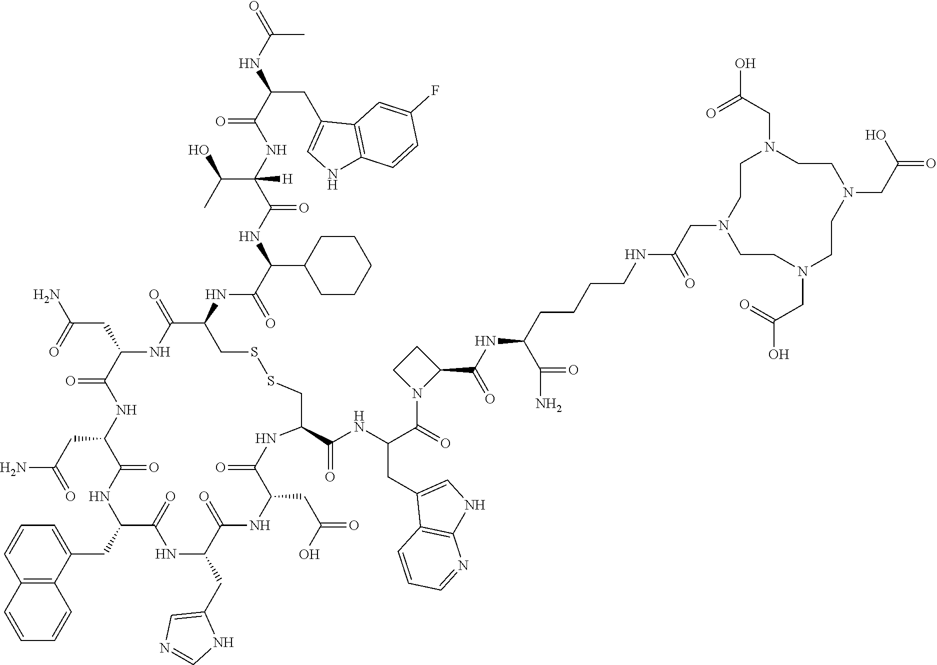 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)NC(Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C1CCCCC1)[C@@H](C)O]

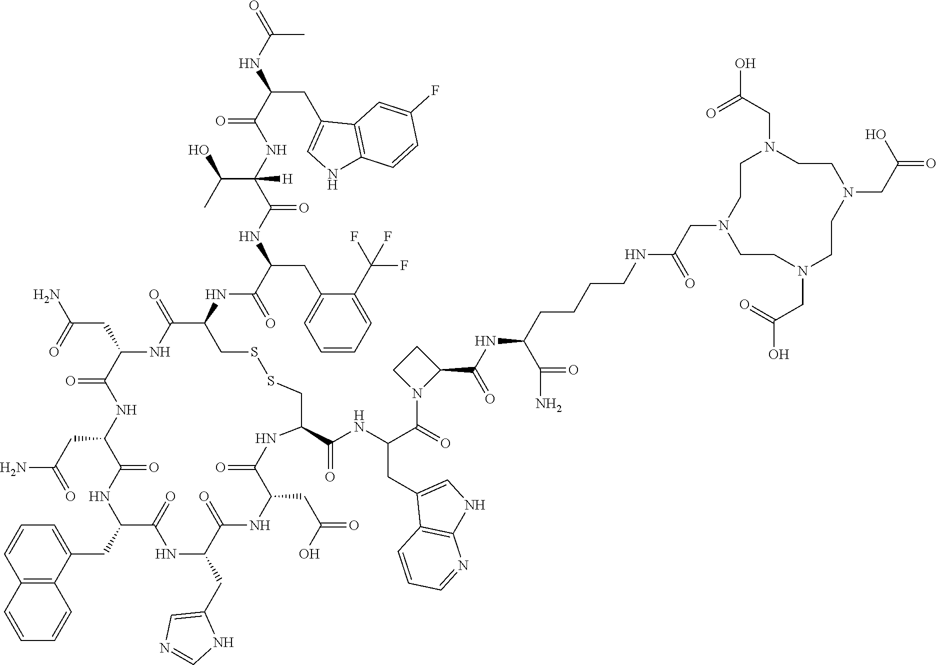 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)NC(Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O]

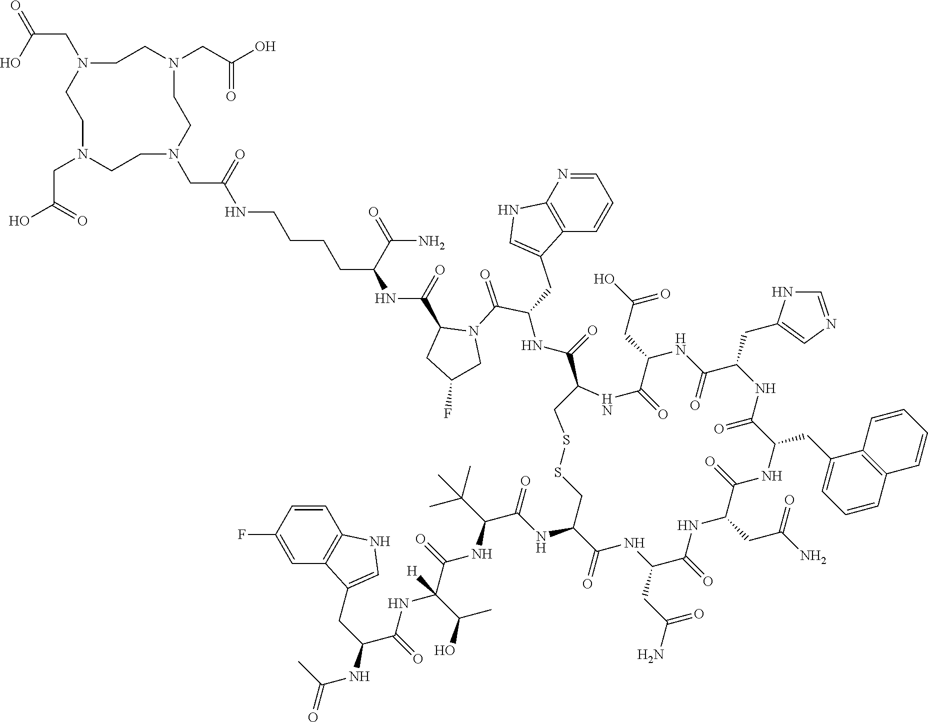 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)C(C)(C)C)[C@@H](C)O]

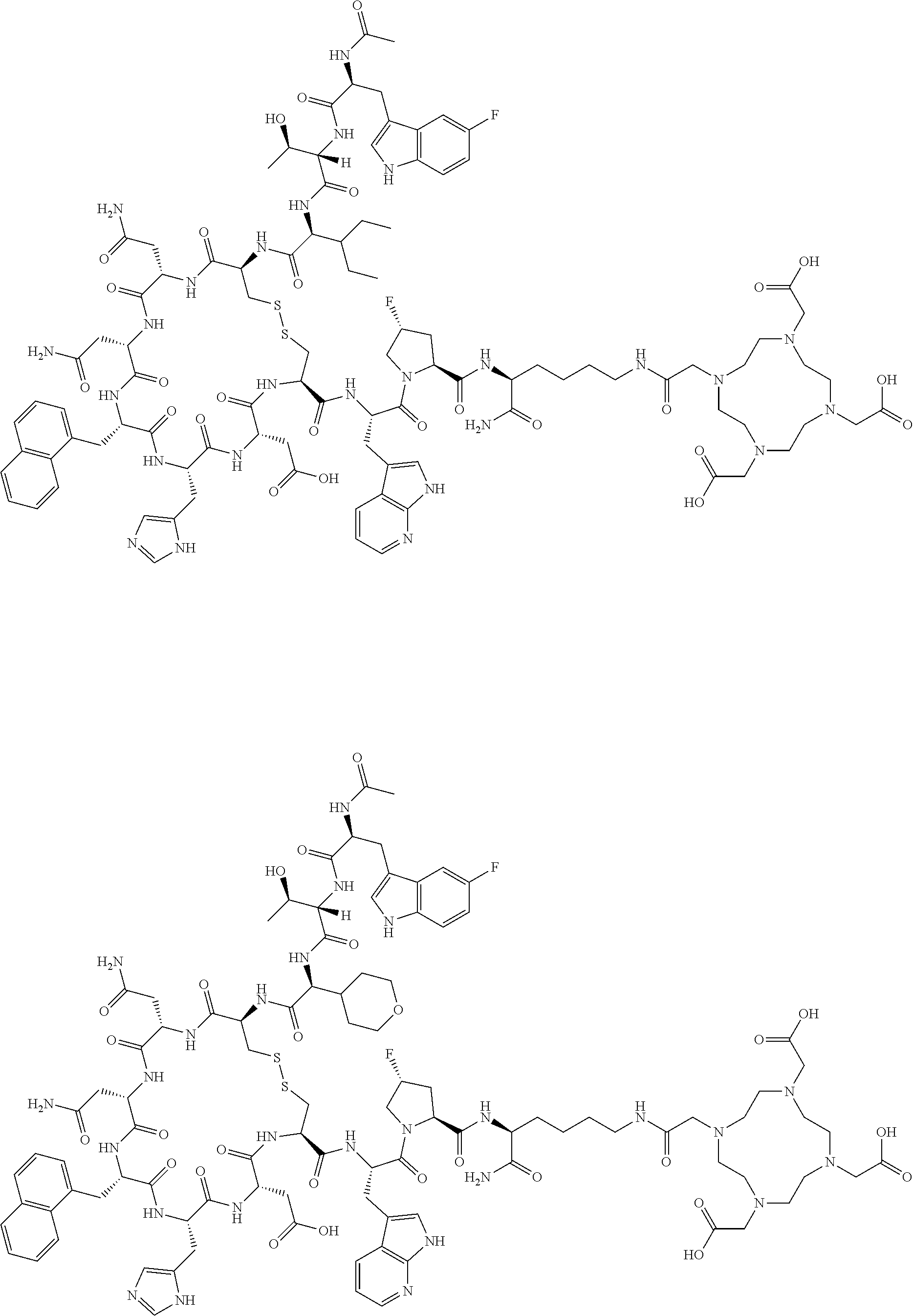

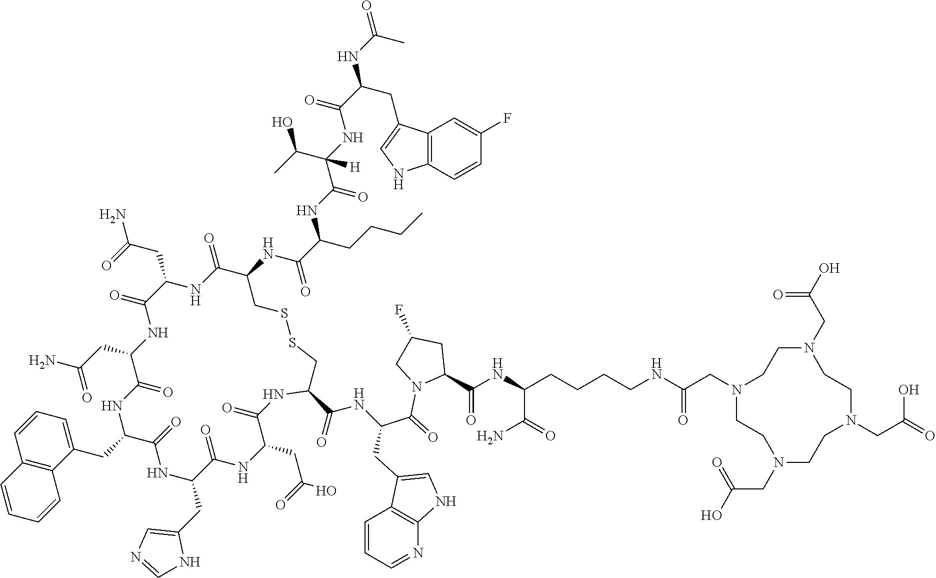 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O]

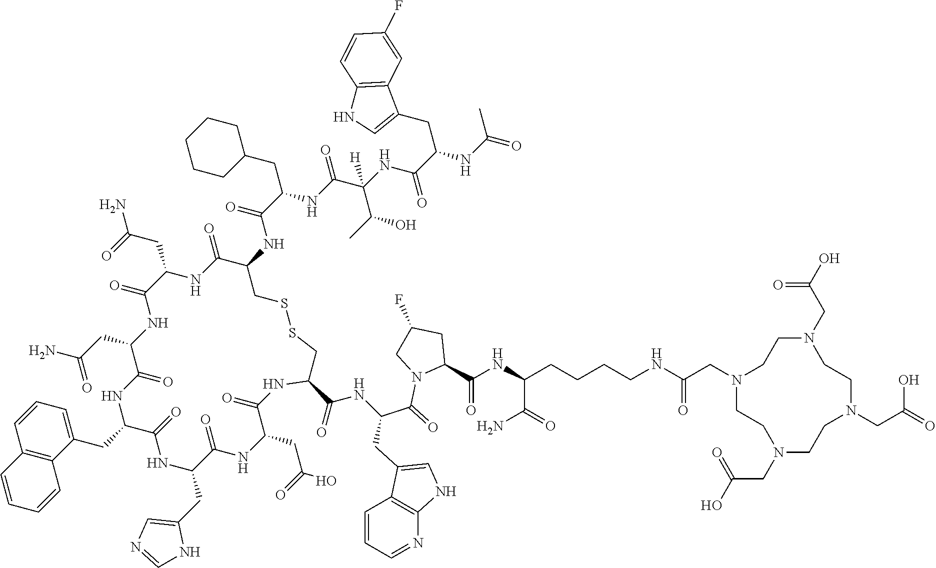 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O]

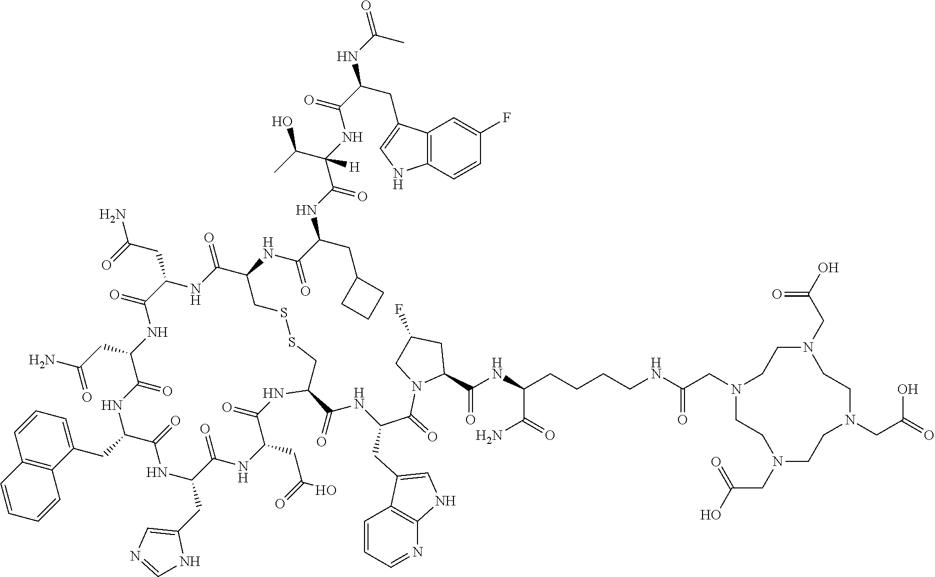 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O)[C@@H](C)O]

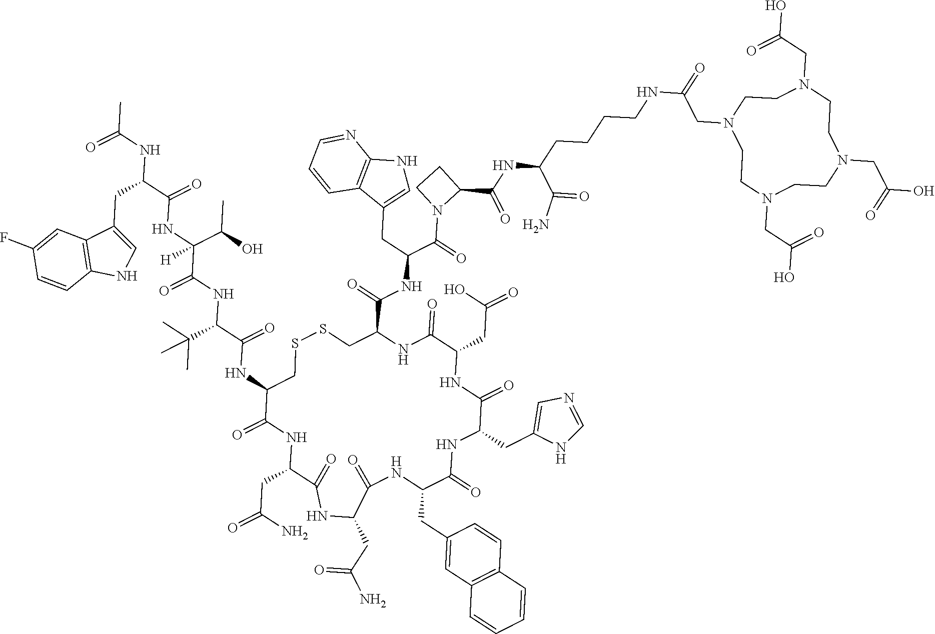 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C(C)(C)C)[C@@H](C)O]

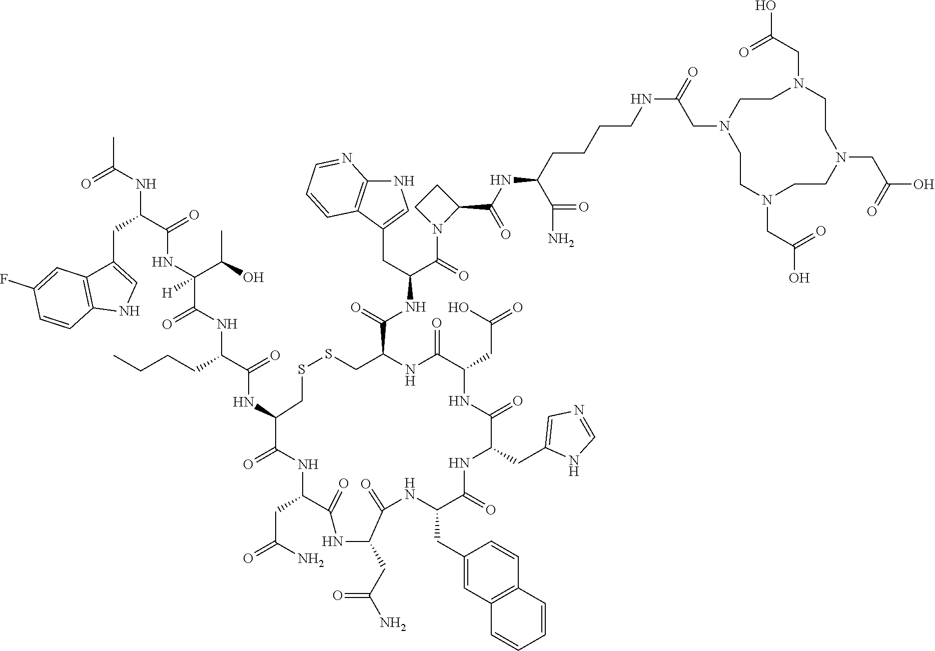 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

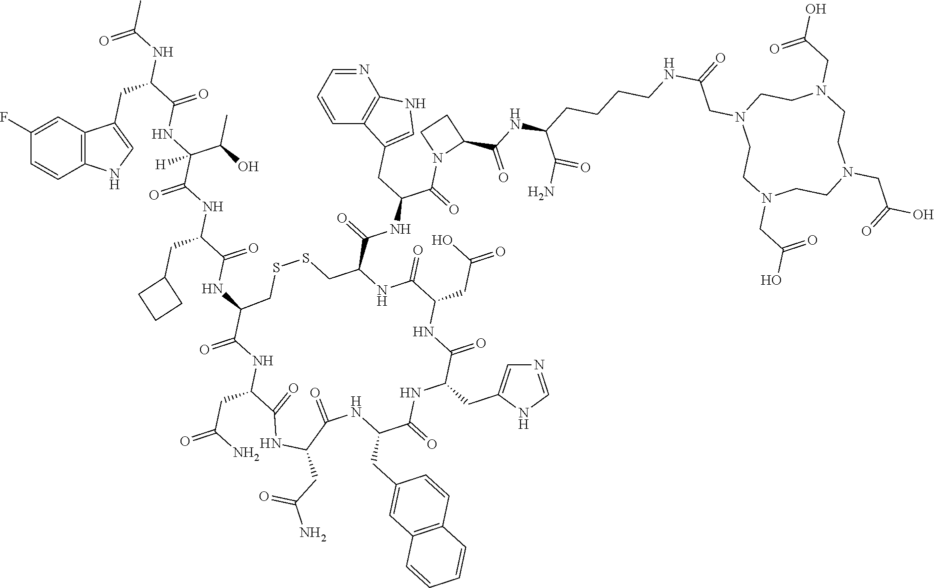 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O]

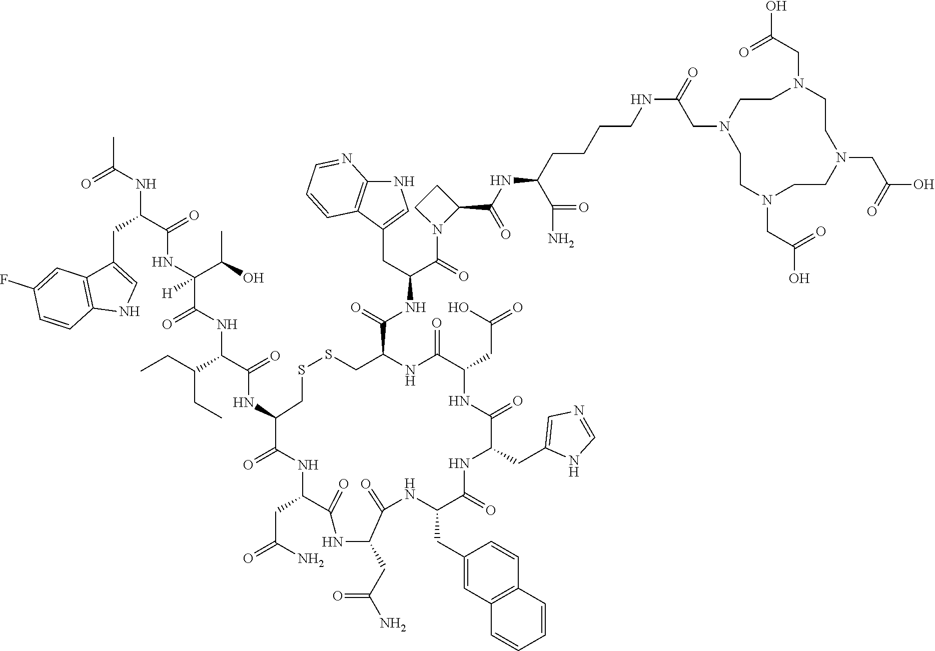 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

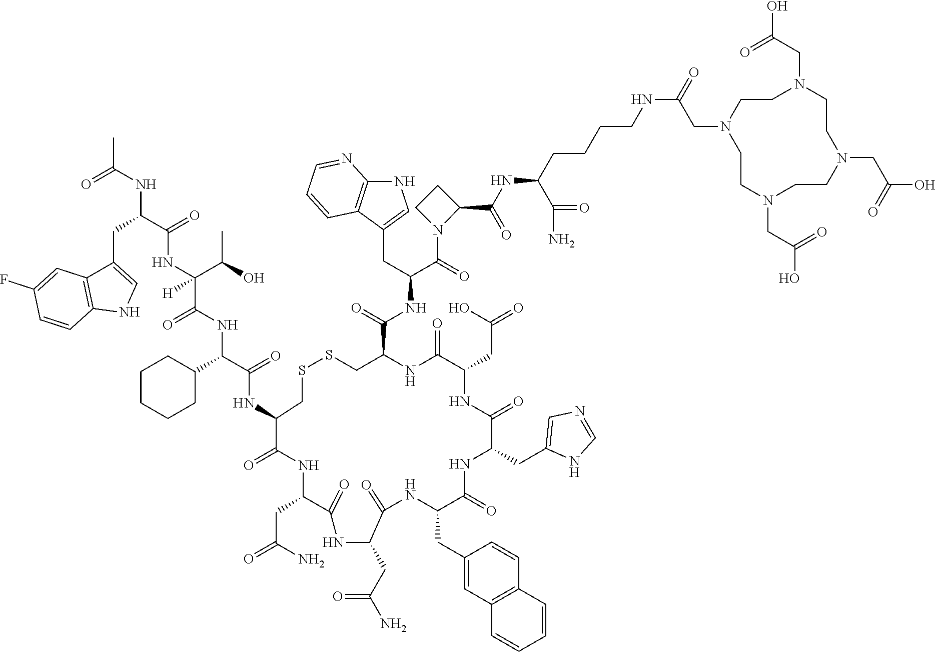 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCCCC1)[C@@H](C)O]

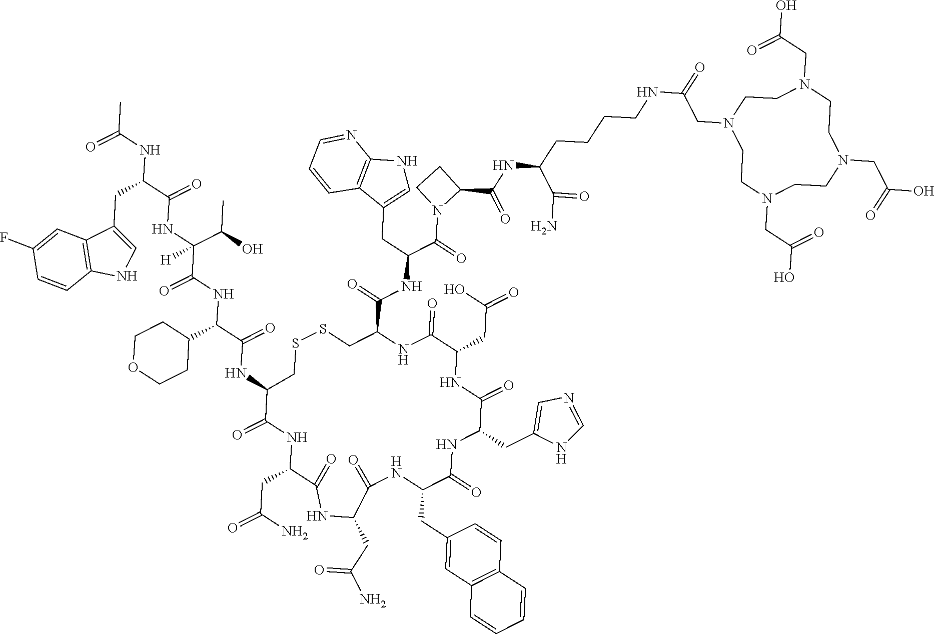 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCOCC1)[C@@H](C)O]

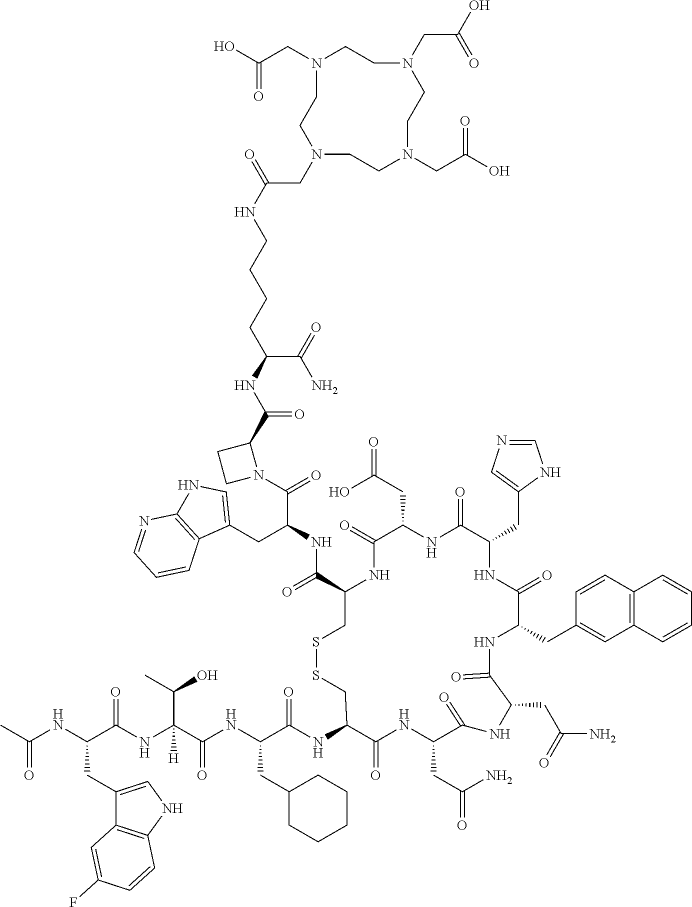 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O]

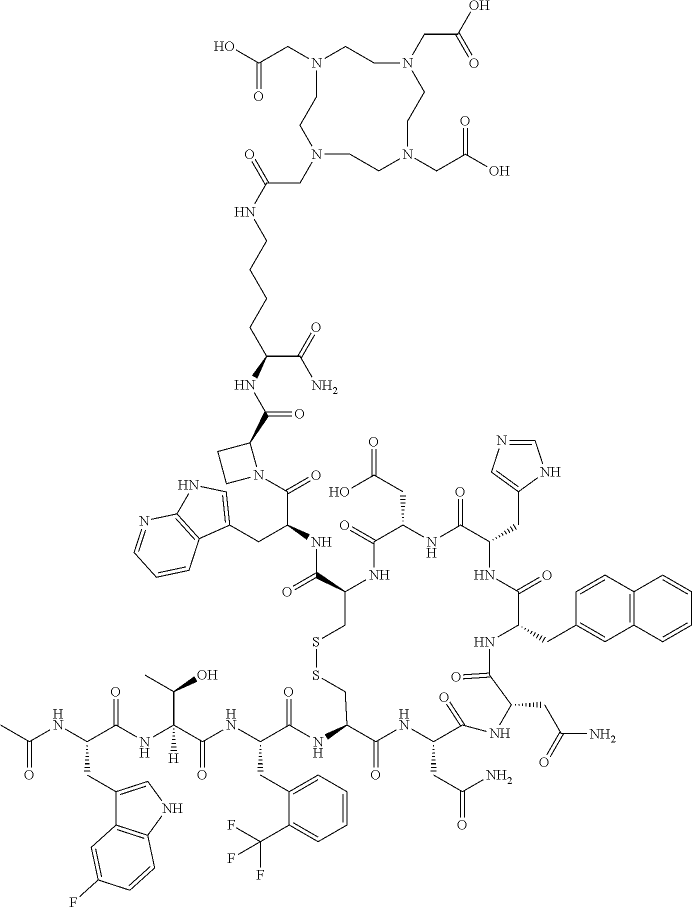 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O]

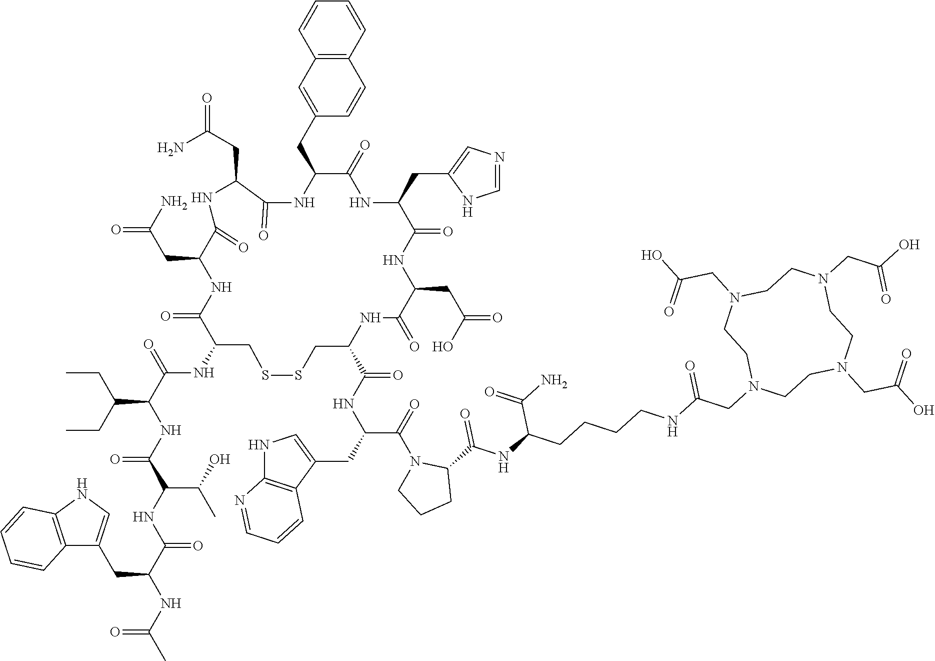

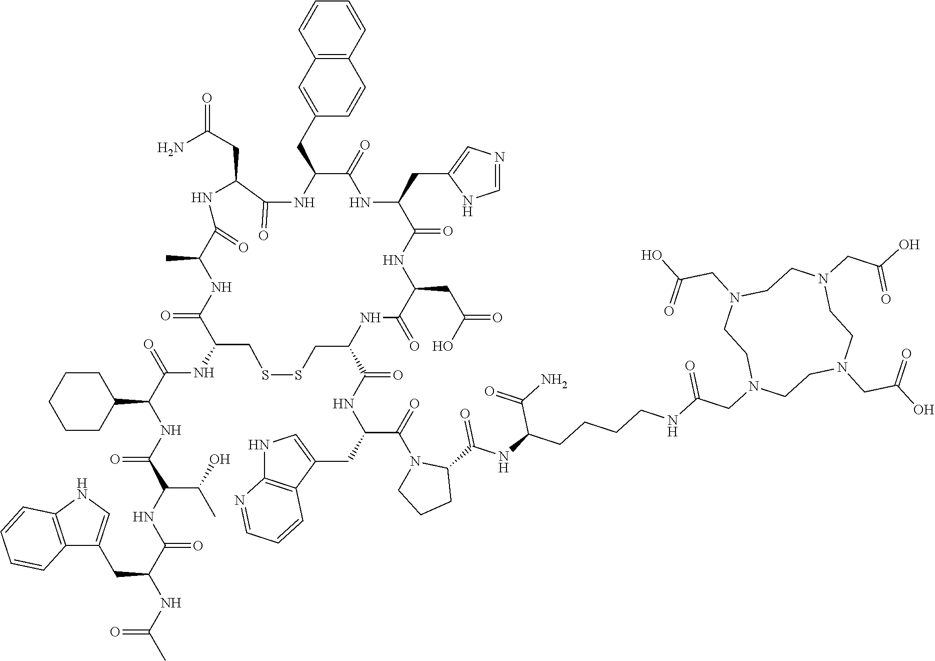

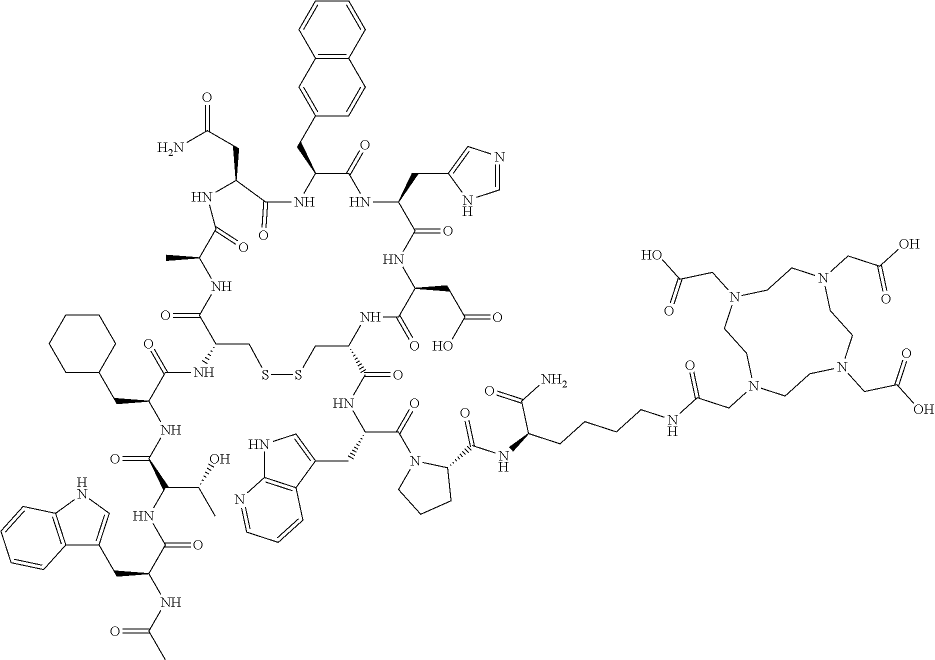

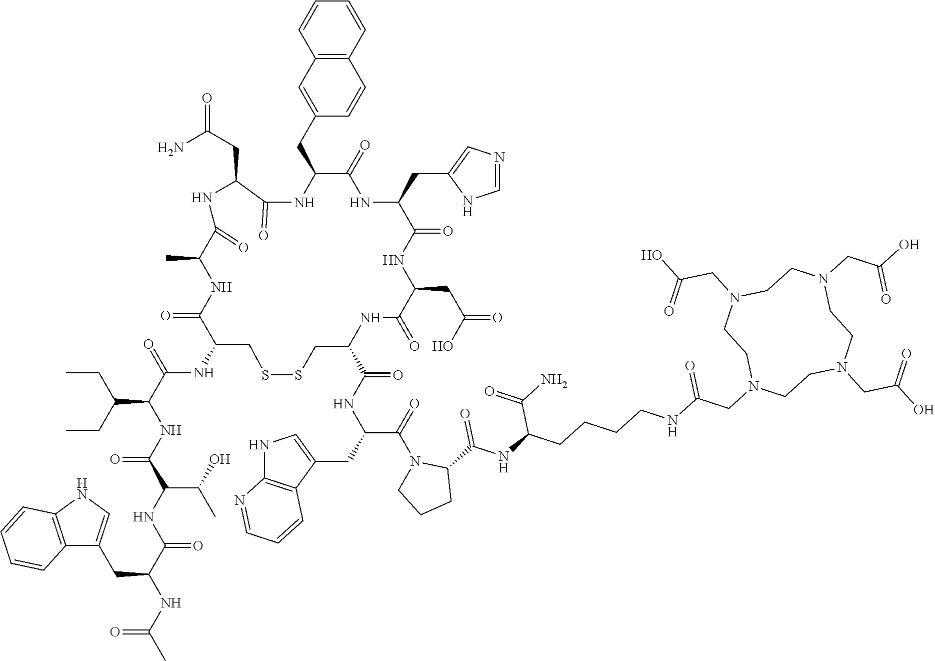

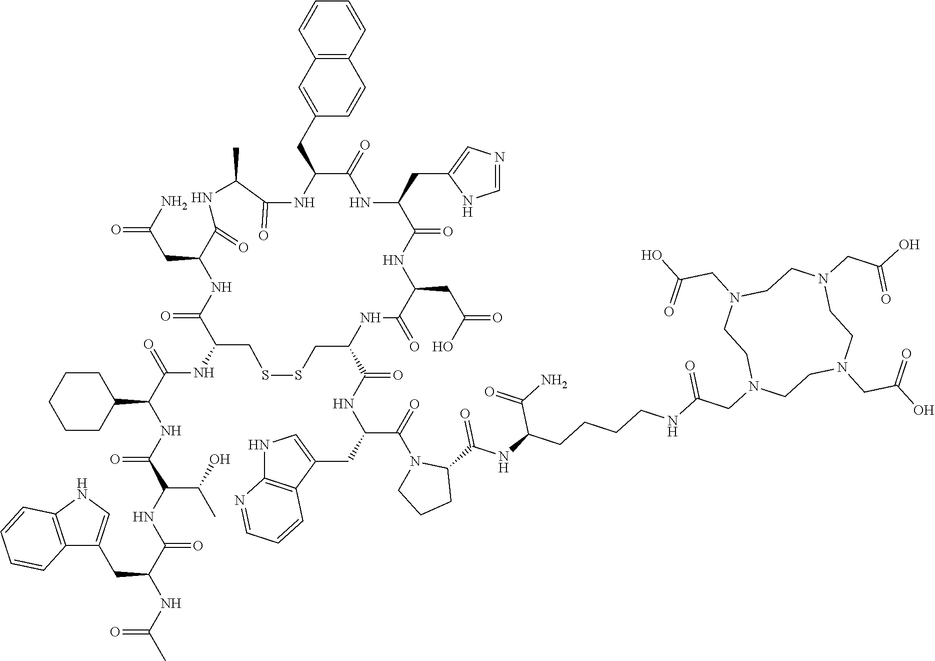

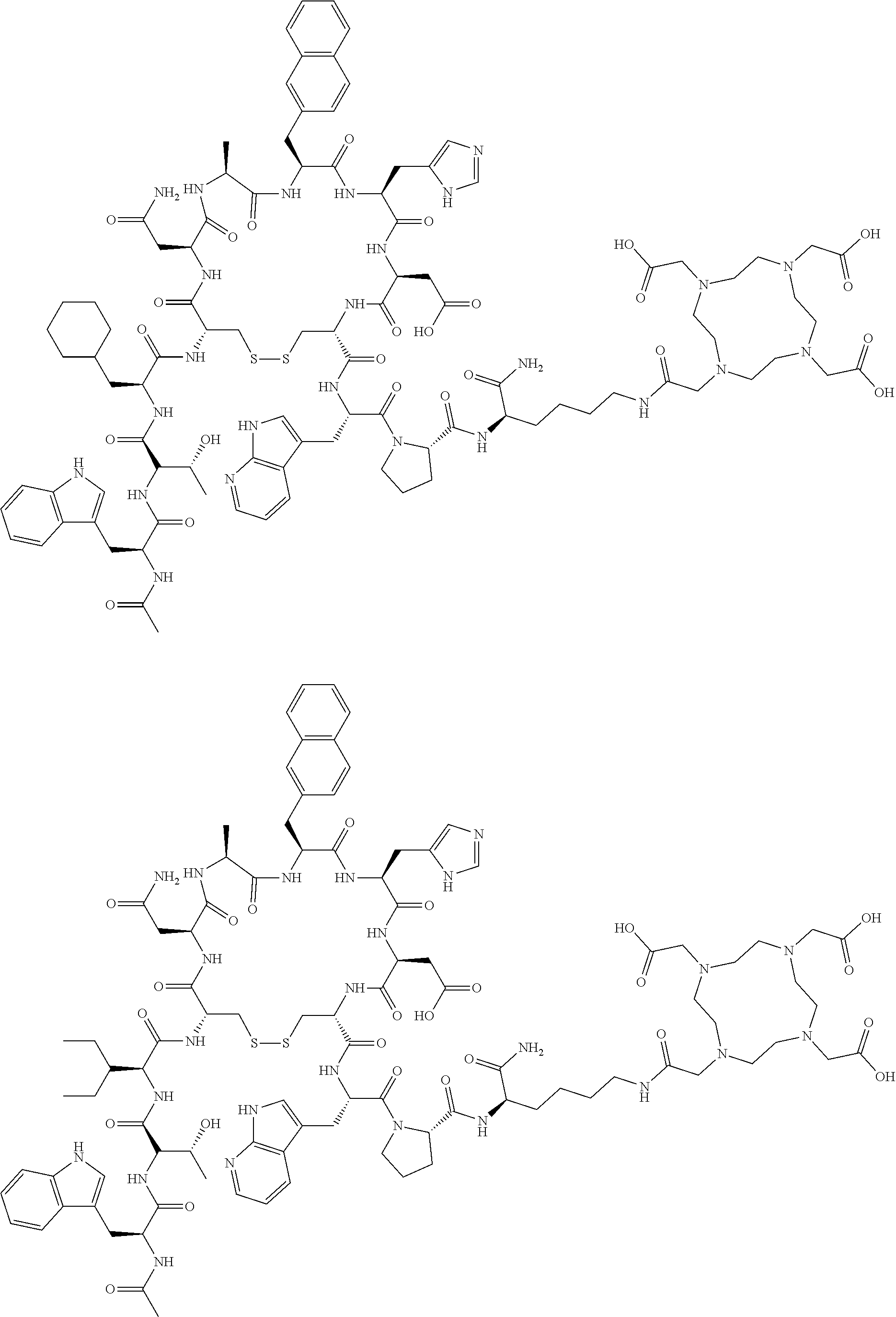

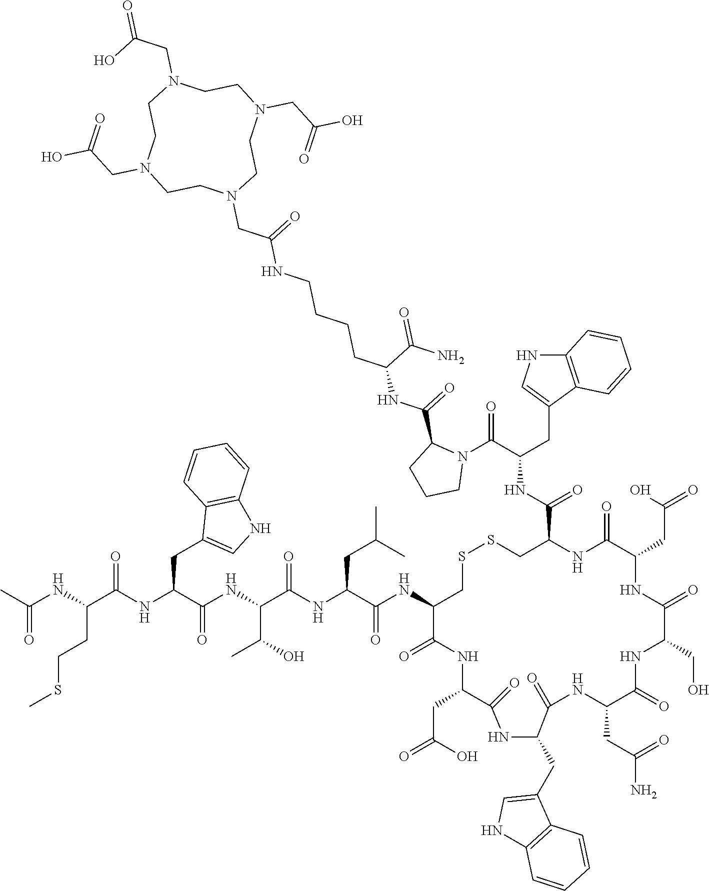 [SMILES: CSCC[C@H](NC(C)=O)C(=O)N[C@@H](Cc1c[nH]c2ccccc12)C(=O)N[C@H](C(=O)N[C@@H](CC(C)C)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](CC(N)=O)NC(=O)[C@H](Cc2c[nH]c3ccccc23)NC(=O)[C@H](CC(=O)O)NC1=O)[C@@H](C)O]

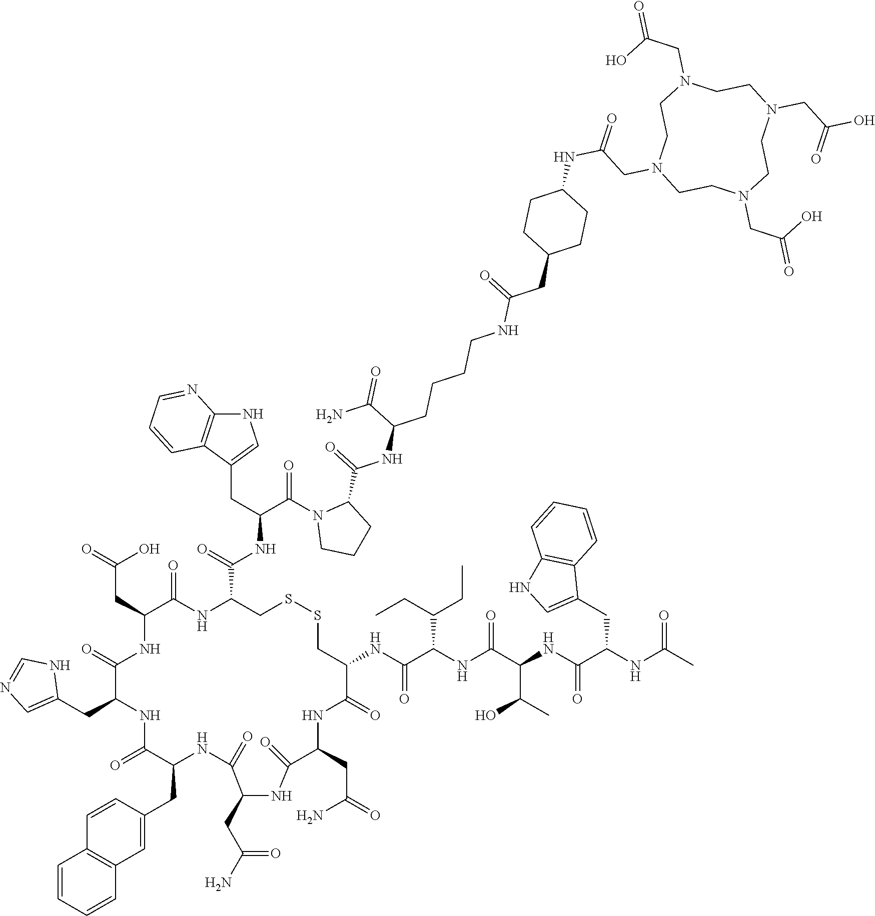 [SMILES: CCC(CC)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)C[C@H]2CC[C@H](NC(=O)CN3CCN(CC(=O)O)CCN(CC(=O)O)CCN(CC(=O)O)CC3)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

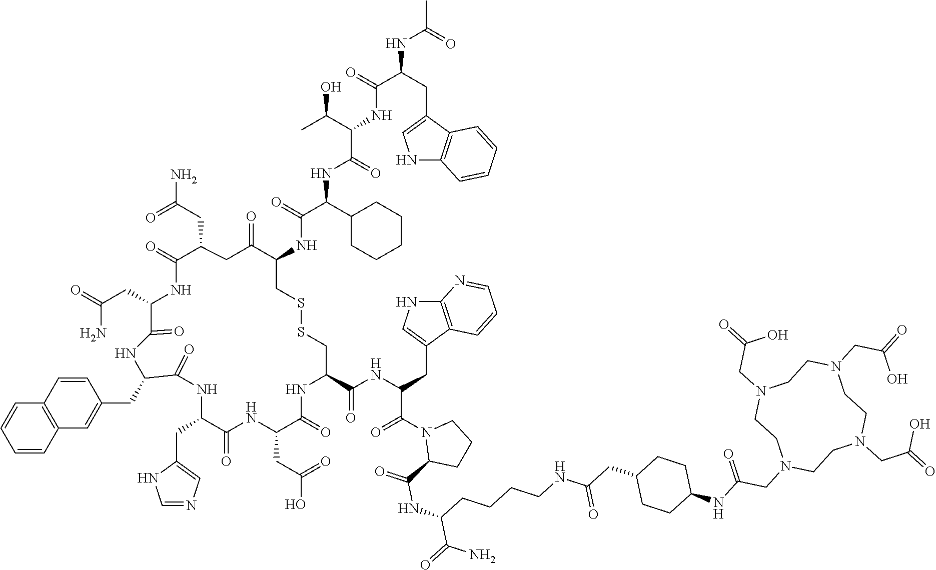

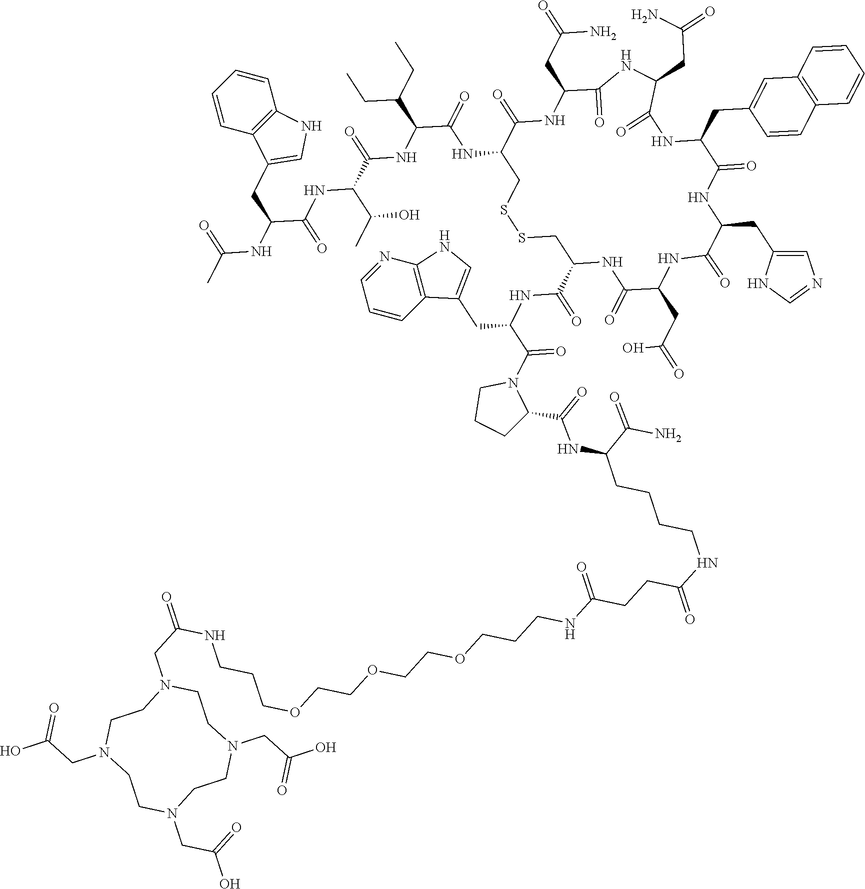 [SMILES: CCC(CC)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ncccc23)C(=O)N2CCC[C@H]2C(=O)N[C@H](CCCCNC(=O)CCC(=O)NCCCOCCOCCOCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

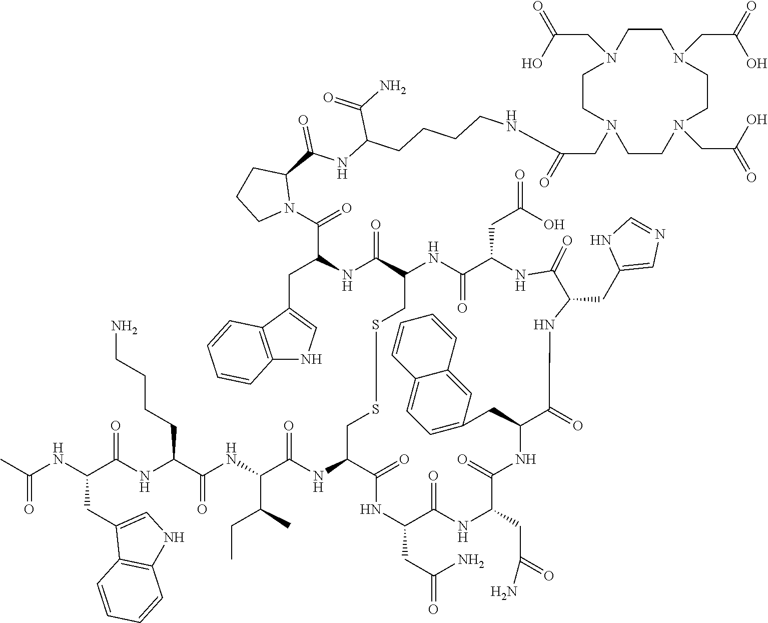

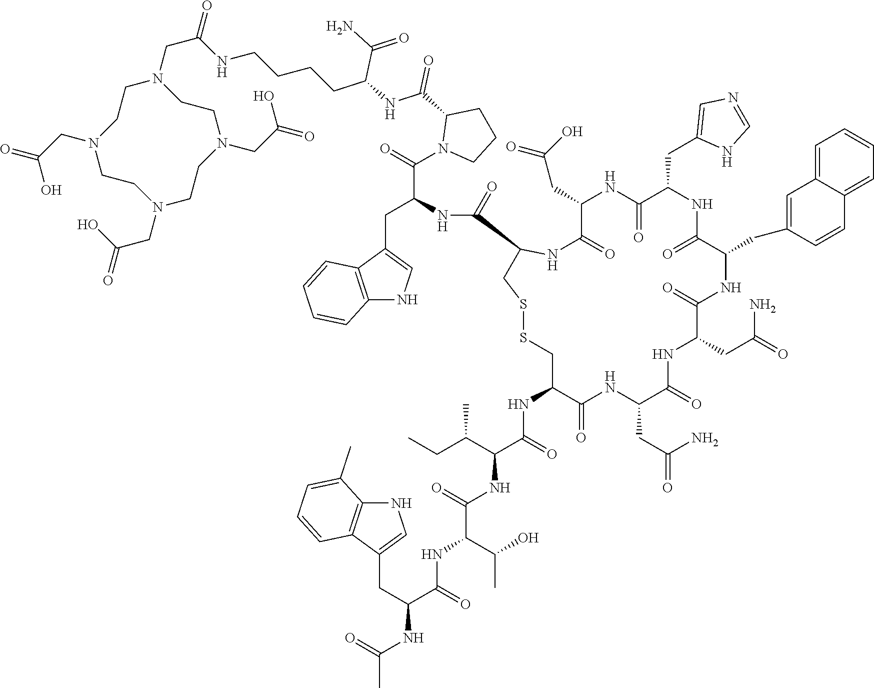 [SMILES: CC[C@H](C)[C@H](NC(=O)[C@@H](NC(=O)[C@H](Cc1c[nH]c2c(C)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

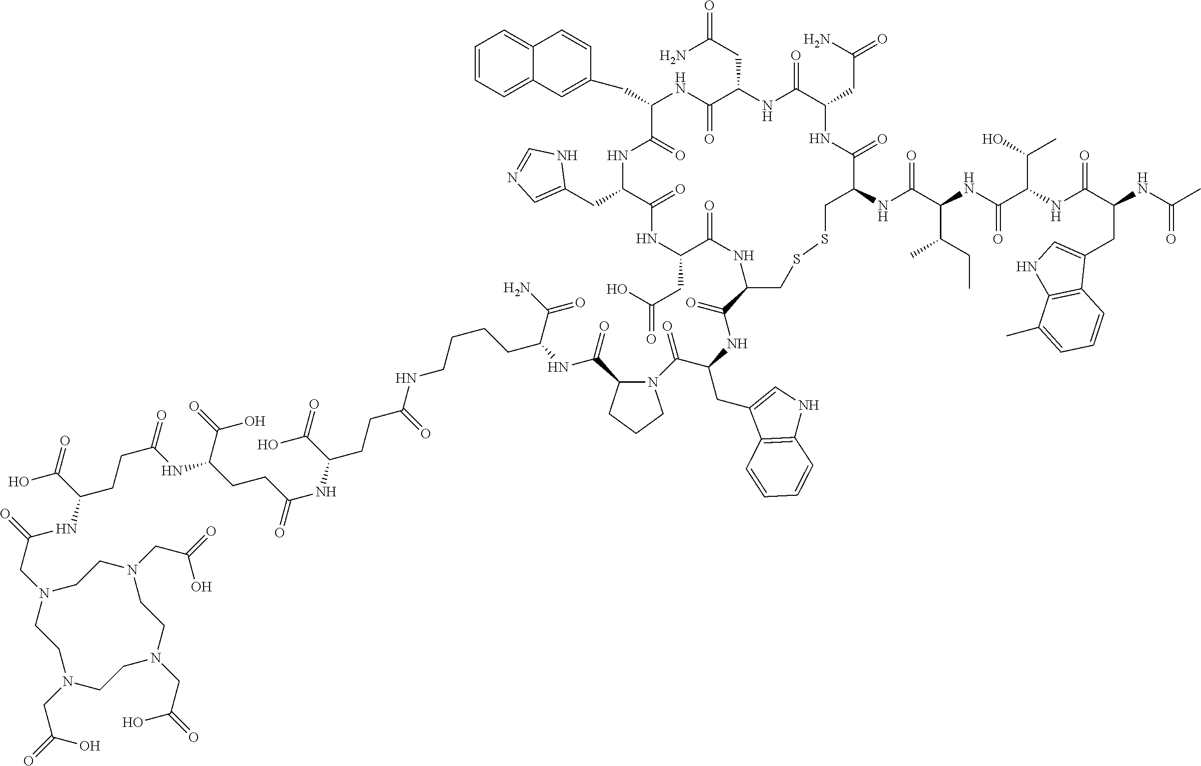 [SMILES: CC[C@H](C)[C@H](NC(=O)[C@@H](NC(=O)[C@H](Cc1c[nH]c2c(C)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

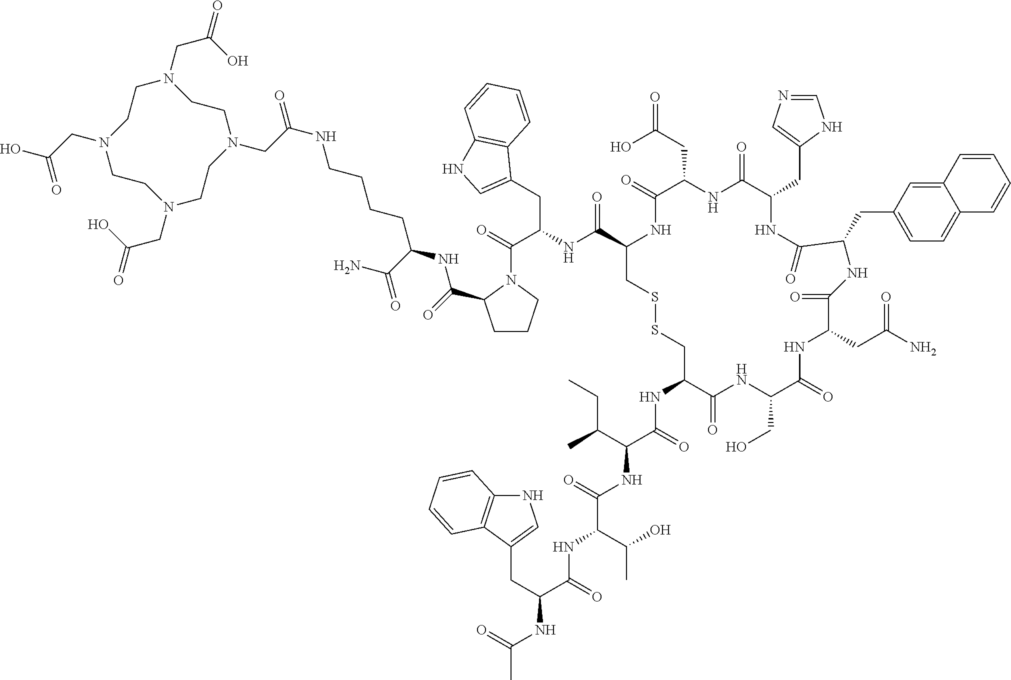 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CO)NC1=O]

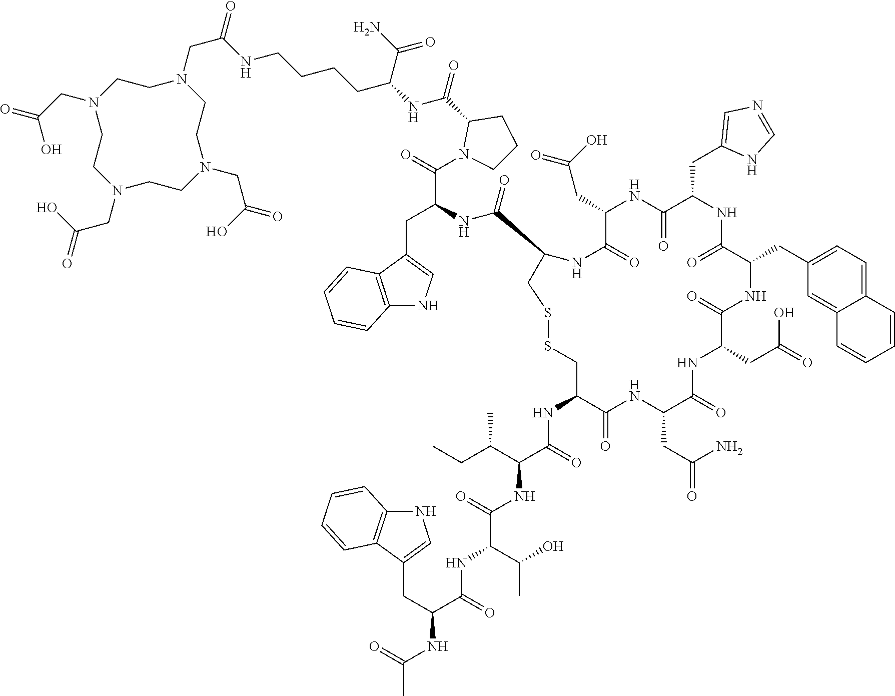 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(=O)O)NC(=O)[C@H](CC(N)=O)NC1=O]

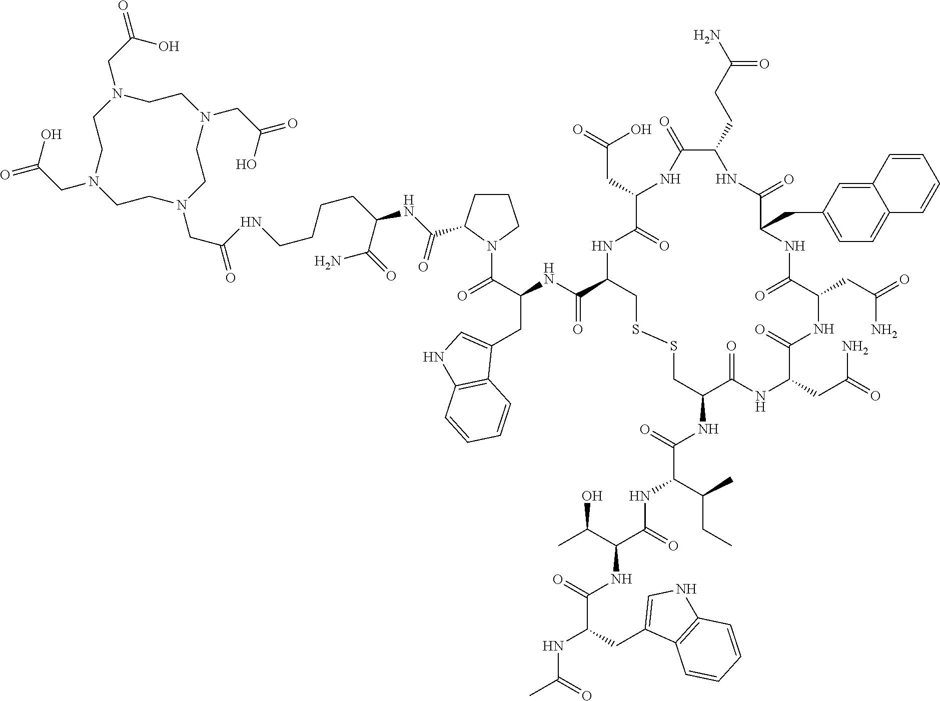 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(N)=O)NC(=O)[C@H](Cc2ccc3ccccc3c2)NC(=O)[C@H](CC(N)=O)NC(=O)[C@H](CC(N)=O)NC1=O]

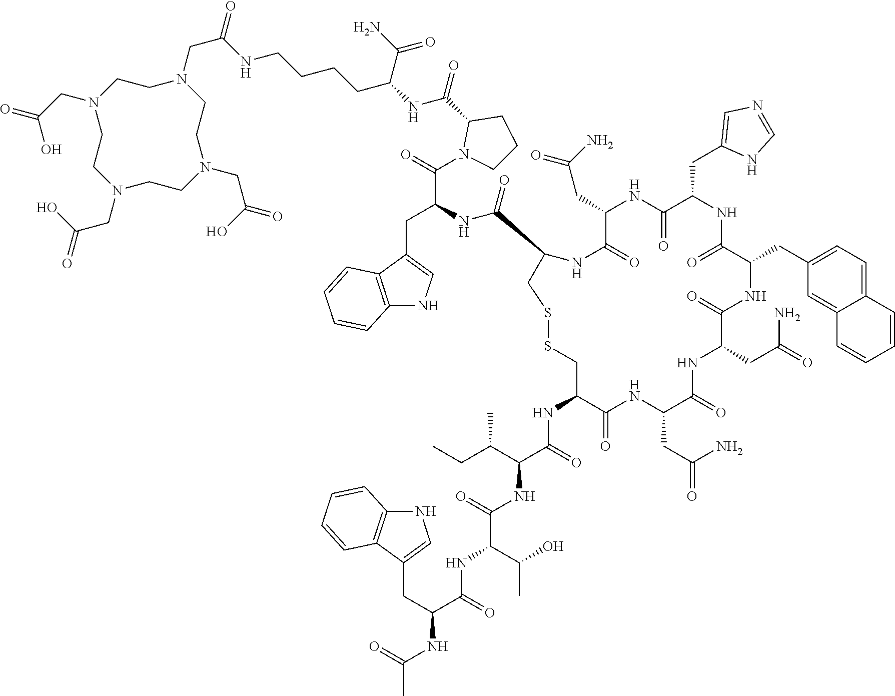 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(N)=O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

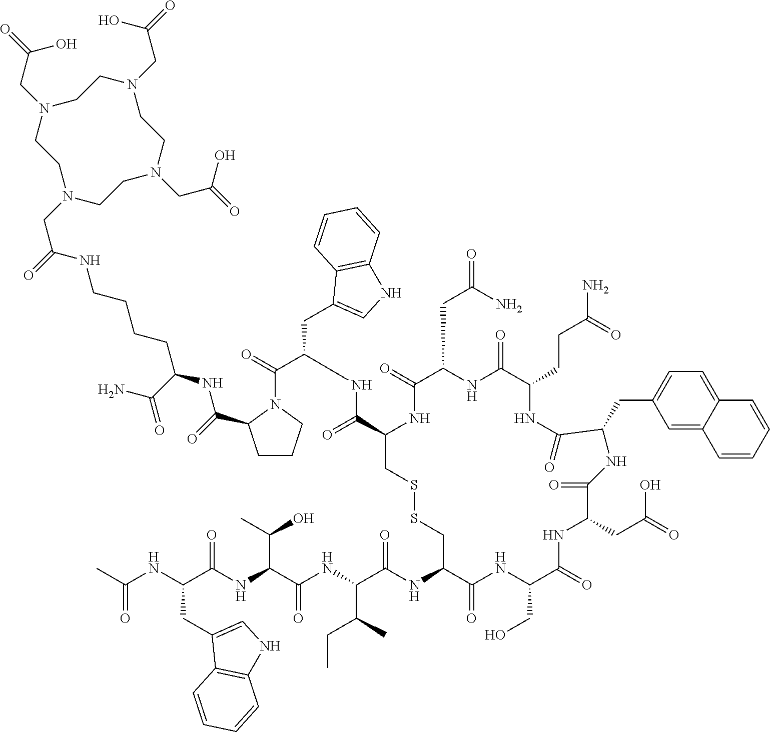 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(N)=O)NC(=O)[C@H](CCC(N)=O)NC(=O)[C@H](Cc2ccc3ccccc3c2)NC(=O)[C@H](CC(=O)O)NC(=O)[C@H](CO)NC1=O]

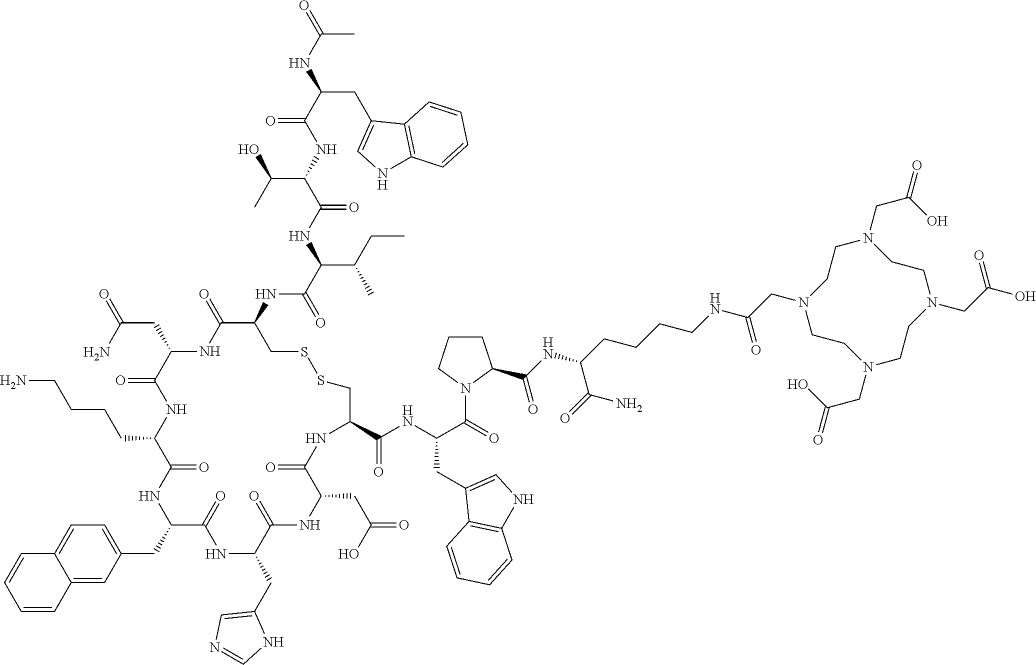 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CCCCN)NC(=O)[C@H](CC(N)=O)NC1=O]

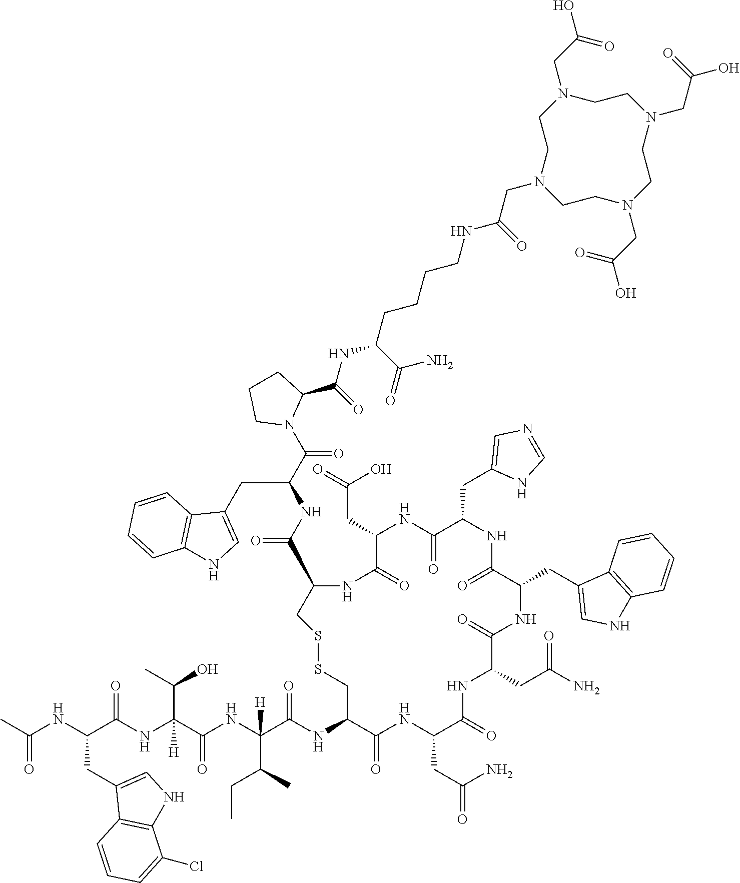 [SMILES: CC[C@H](C)[C@H](NC(=O)[C@@H](NC(=O)[C@H](Cc1c[nH]c2c(Cl)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

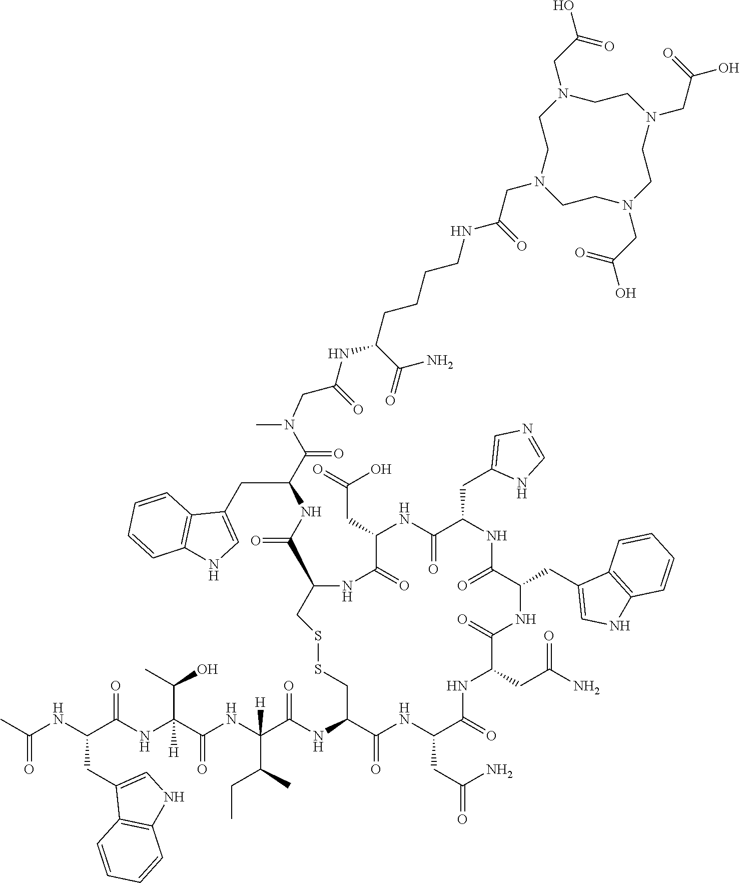 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)CC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

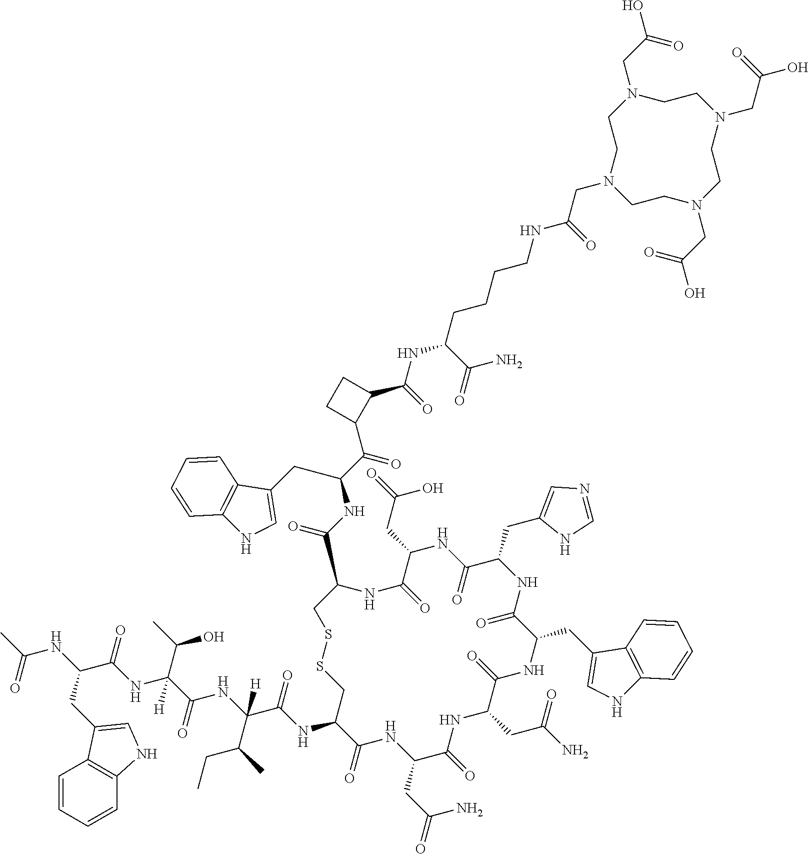

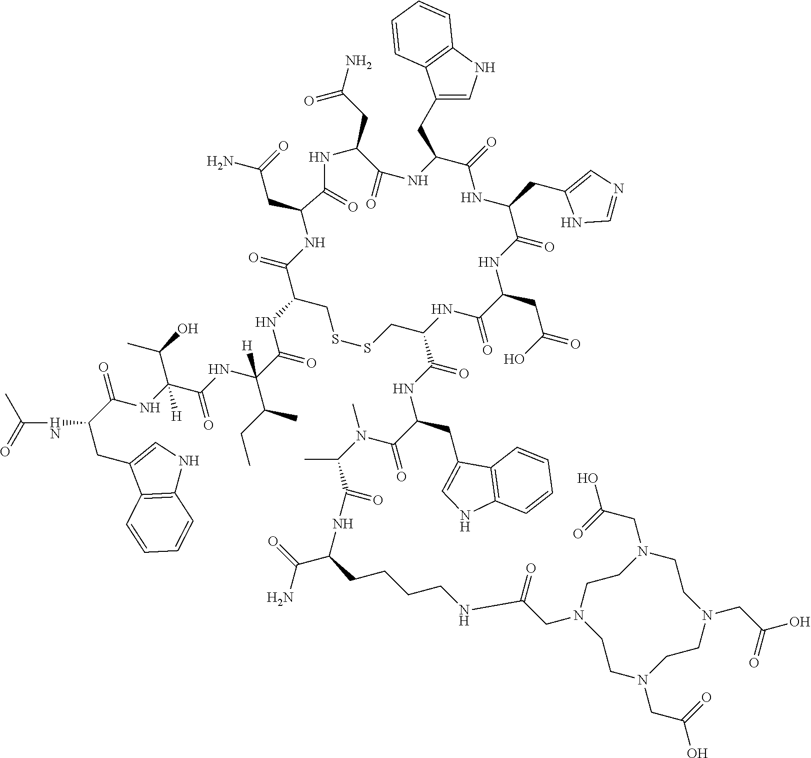 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)[C@@H](C)C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

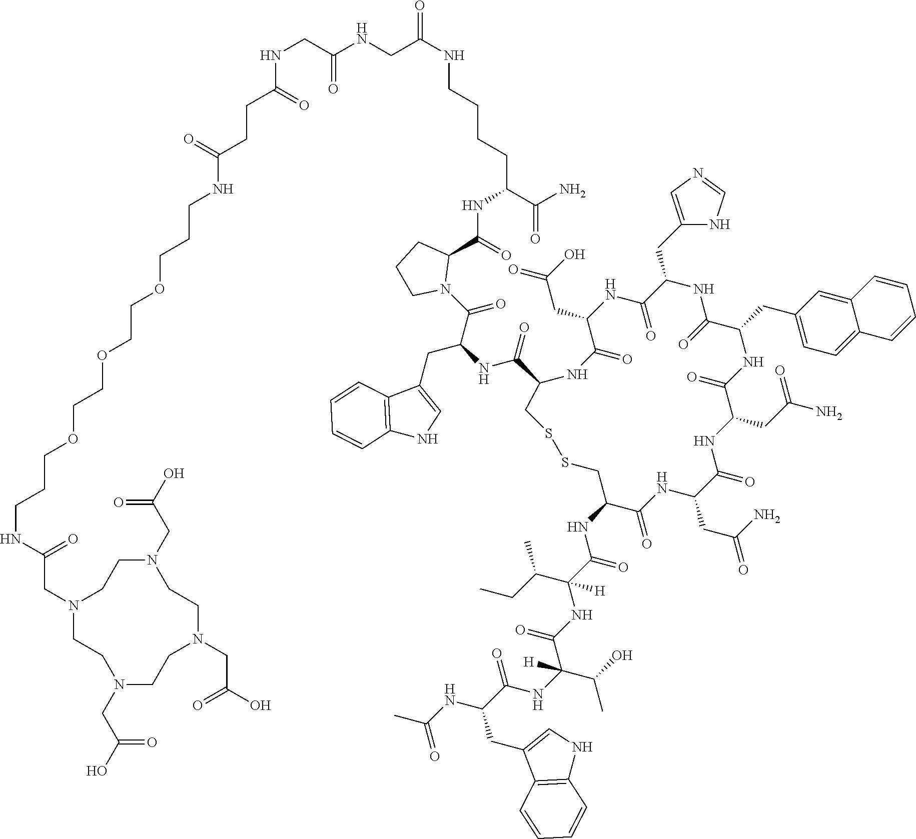 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CNC(=O)CNC(=O)CCC(=O)NCCCOCCOCCOCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

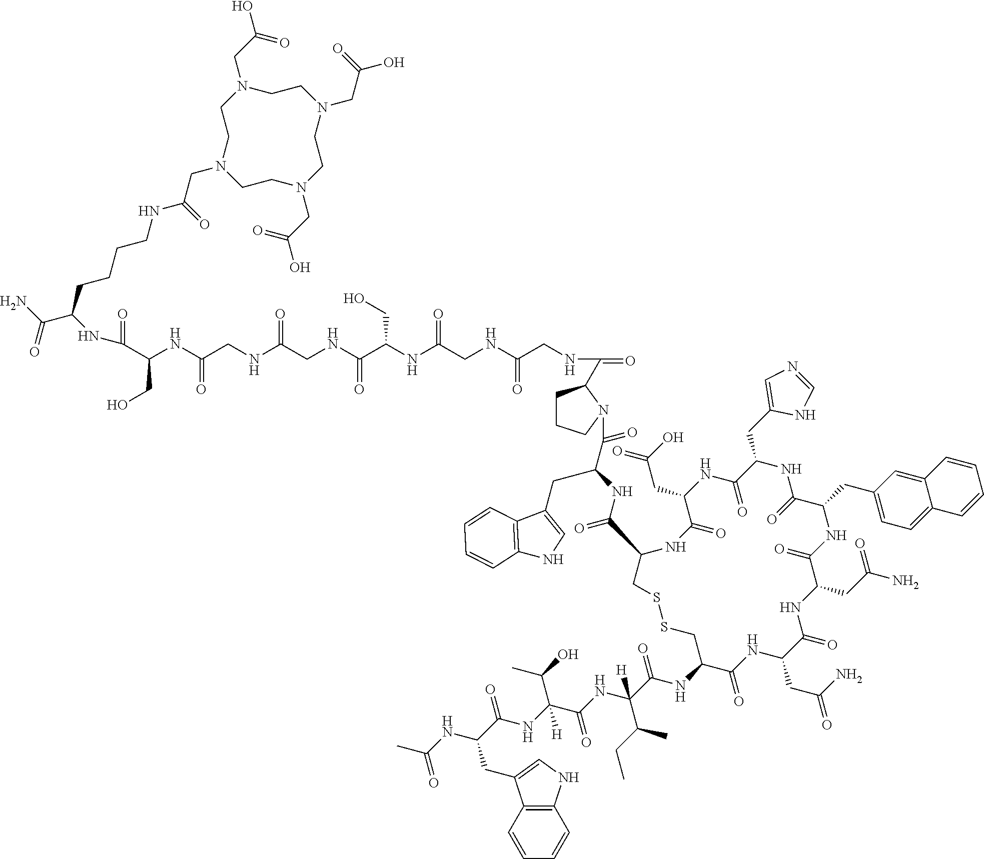 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCC(=O)NCC(=O)N[C@@H](CO)C(=O)NCC(=O)NCC(=O)N[C@@H](CO)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

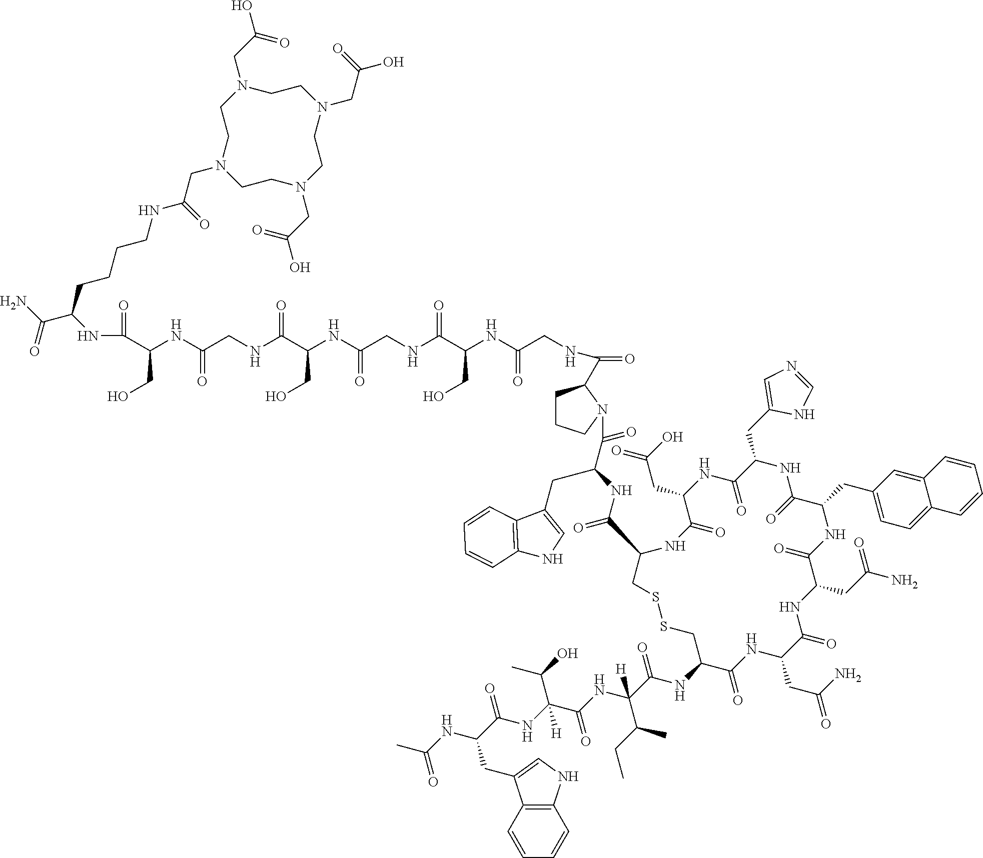 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCC(=O)N[C@@H](CO)C(=O)NCC(=O)N[C@@H](CO)C(=O)NCC(=O)N[C@@H](CO)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

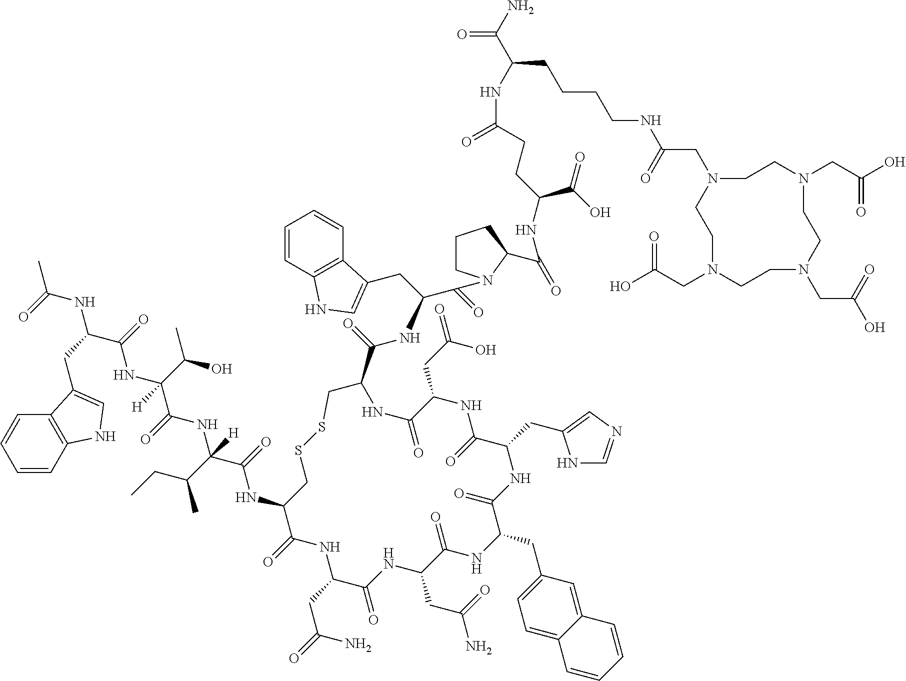 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@@H](CCC(=O)N[C@H](CCCCNC(=O)CN2CCN(CC(=O)O)CCN(CC(=O)O)CCN(CC(=O)O)CC2)C(N)=O)C(=O)O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

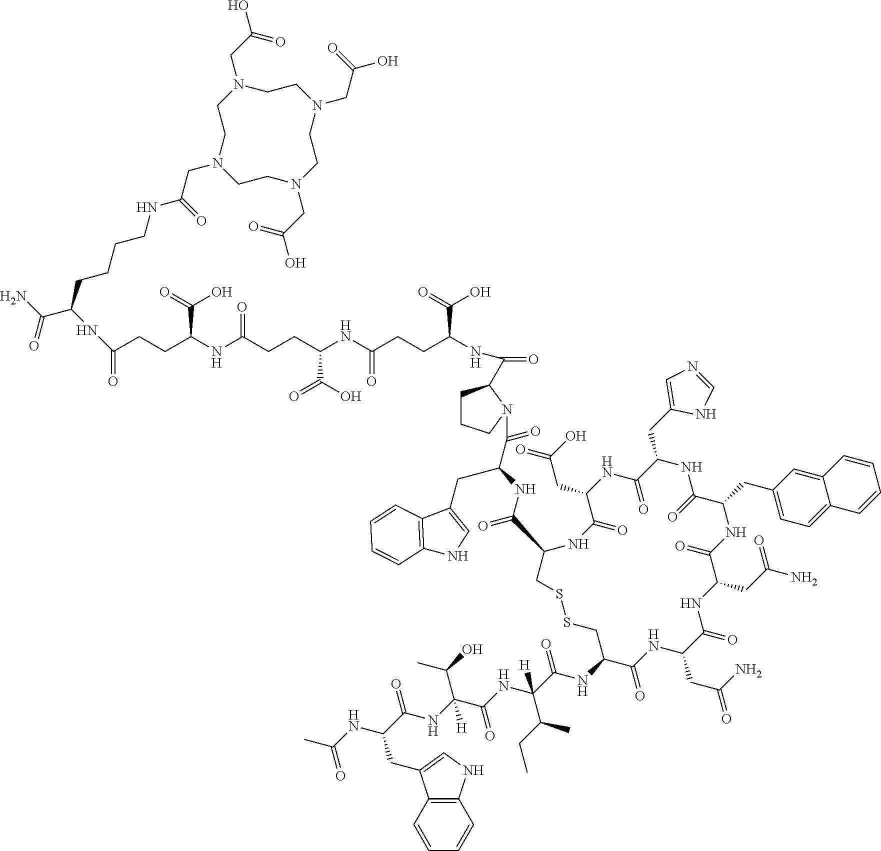 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@@H](CCC(=O)N[C@@H](CCC(=O)N[C@@H](CCC(=O)N[C@H](CCCCNC(=O)CN2CCN(CC(=O)O)CCN(CC(=O)O)CCN(CC(=O)O)CC2)C(N)=O)C(=O)O)C(=O)O)C(=O)O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

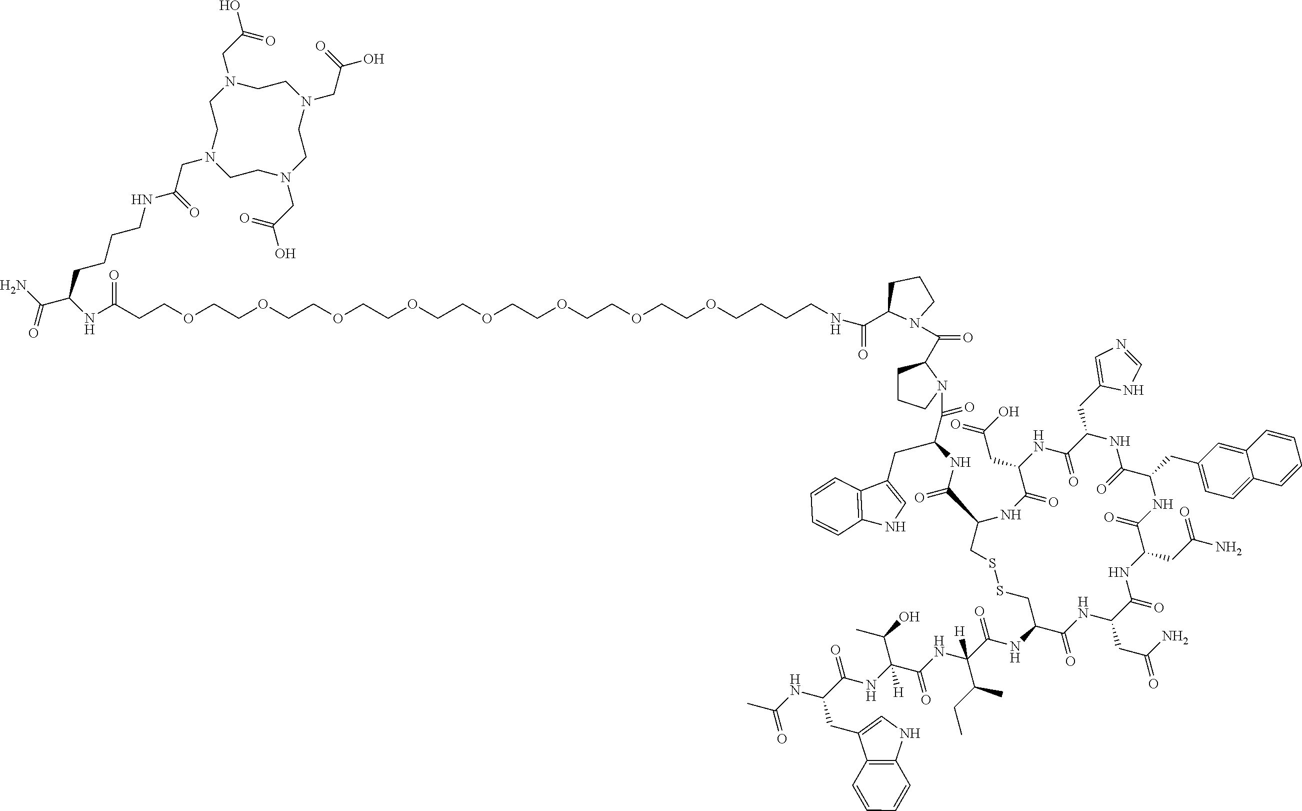 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N2CCC[C@@H]2C(=O)NCCCCOCCOCCOCCOCCOCCOCCOCCOCCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

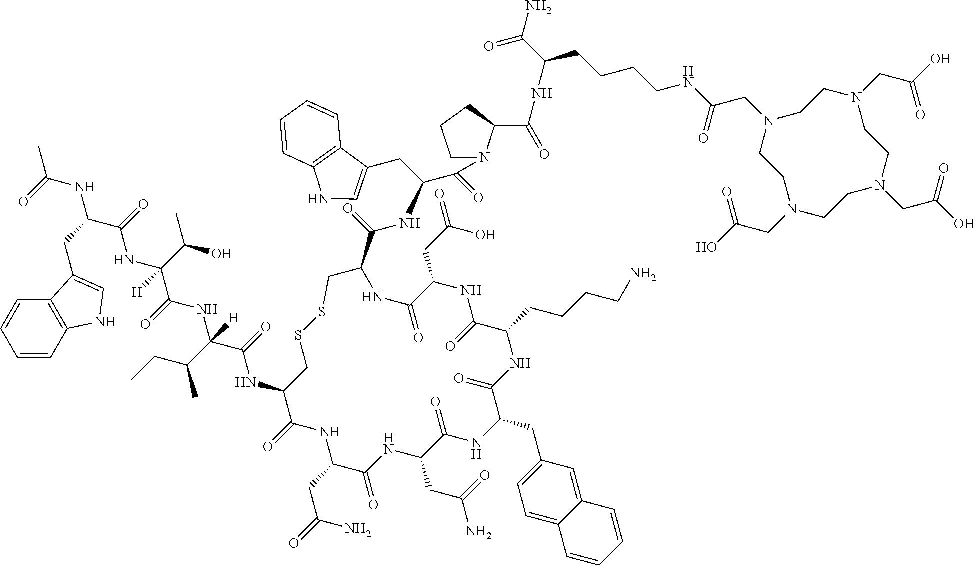 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCCCN)NC(=O)[C@H](Cc2ccc3ccccc3c2)NC(=O)[C@H](CC(N)=O)NC(=O)[C@H](CC(N)=O)NC1=O]

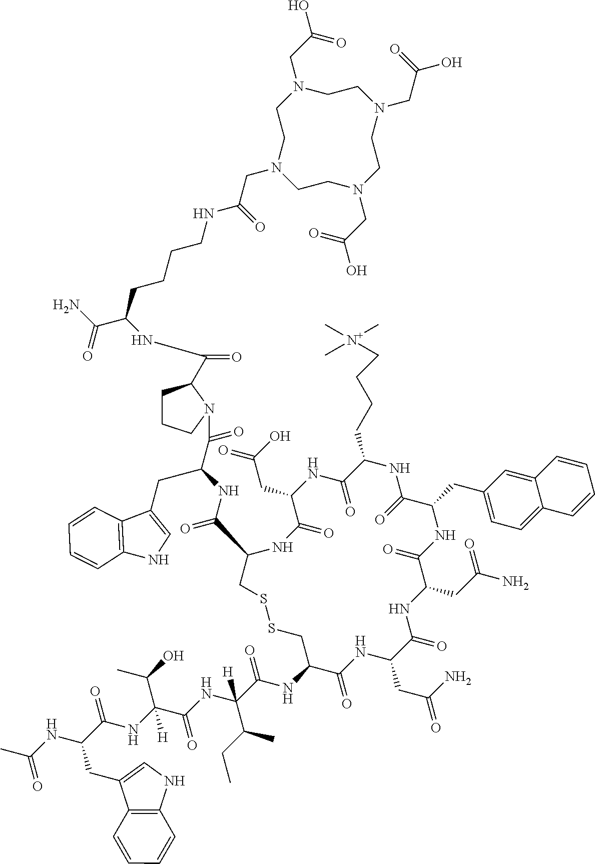 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCCC[N+](C)(C)C)NC(=O)[C@H](Cc2ccc3ccccc3c2)NC(=O)[C@H](CC(N)=O)NC(=O)[C@H](CC(N)=O)NC1=O]

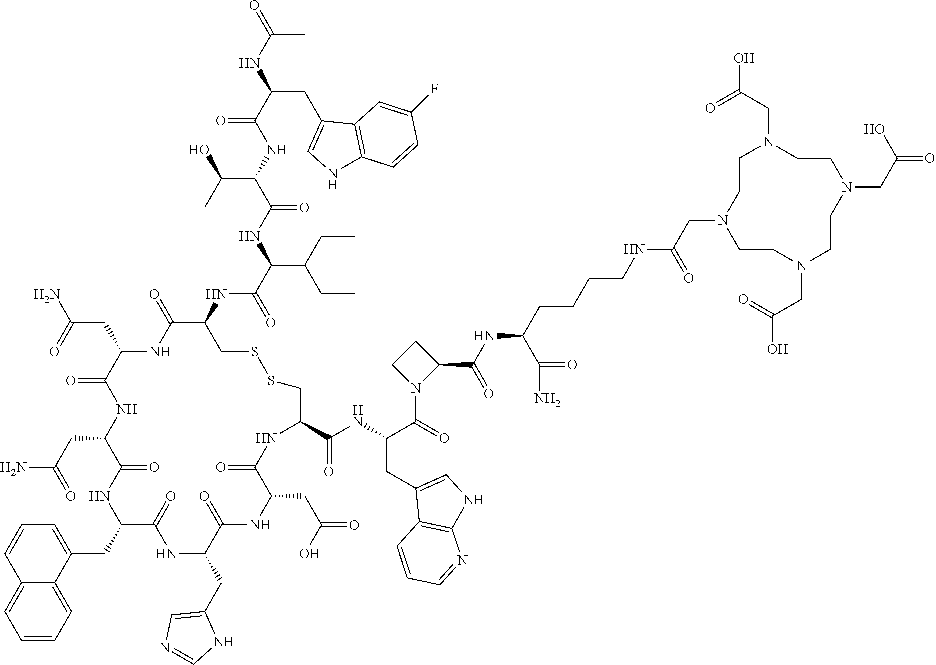 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2CC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2cccc3ccccc23)NC(=O)[C@H](CC(N)=O)NC(=O)[C@H](CC(N)=O)NC1=O]

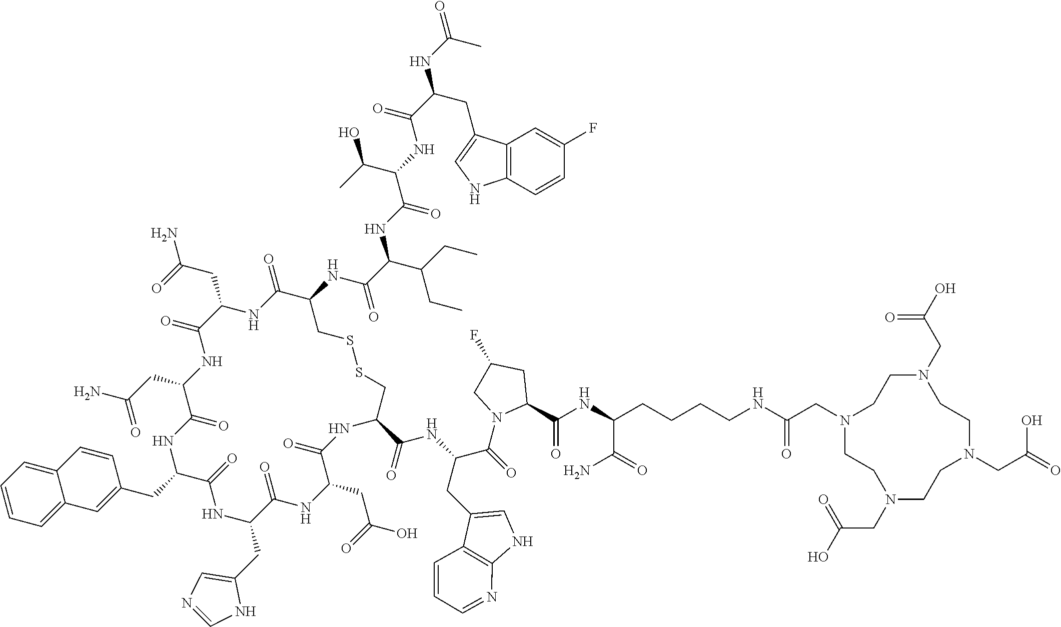 [SMILES: CCC(CC)[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

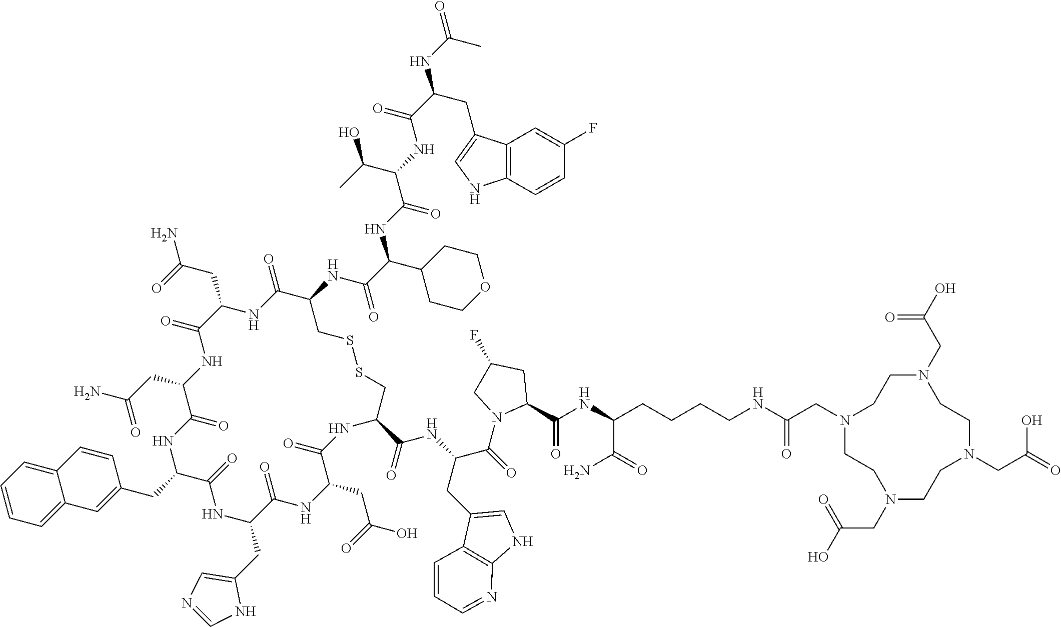 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCOCC1)[C@@H](C)O]

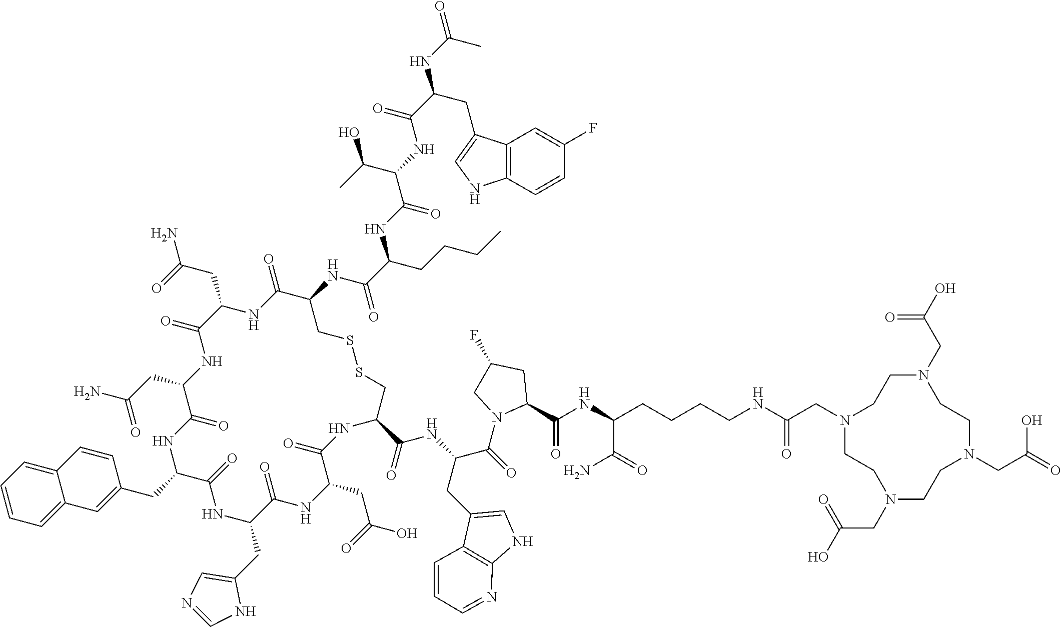 [SMILES: CCCC[C@H](NC(=O)[C@@H](NC(=O)[C@H](Cc1c[nH]c2ccc(F)cc12)NC(C)=O)[C@@H](C)O)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

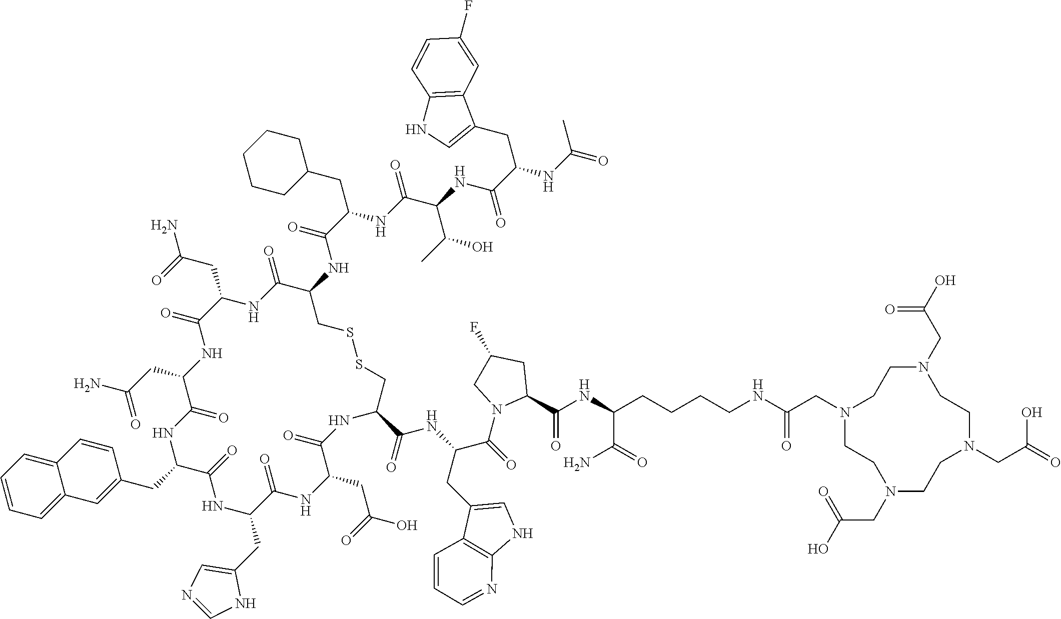 [SMILES: CC(=O)N[C@@H](Cc1c[nH]c2ccc(F)cc12)C(=O)N[C@H](C(=O)N[C@@H](CC1CCCCC1)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O]

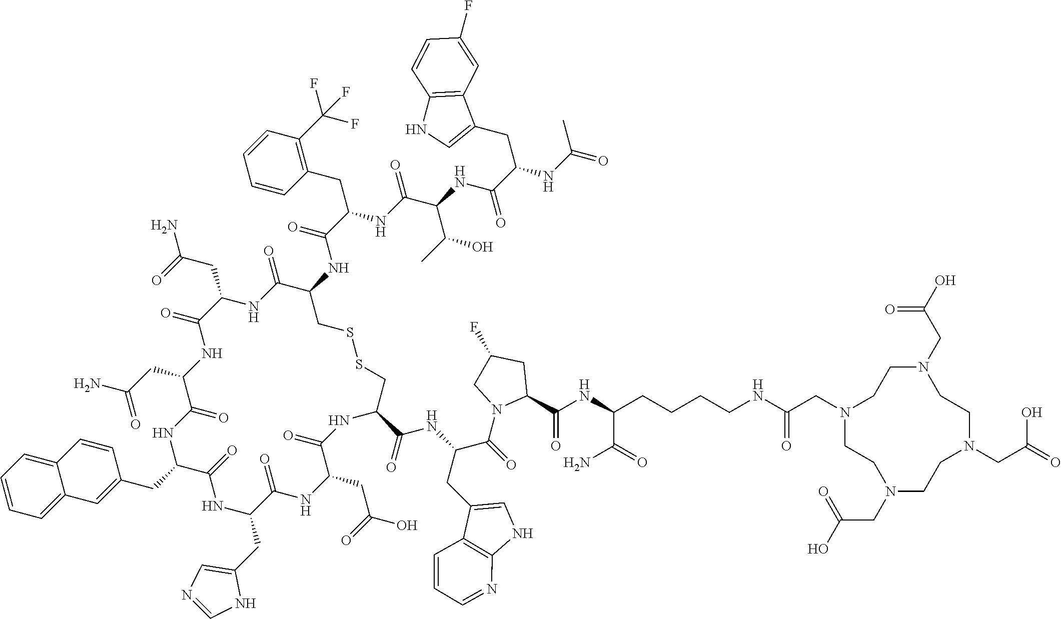 [SMILES: CC(=O)N[C@@H](Cc1c[nH]c2ccc(F)cc12)C(=O)N[C@H](C(=O)N[C@@H](Cc1ccccc1C(F)(F)F)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O]

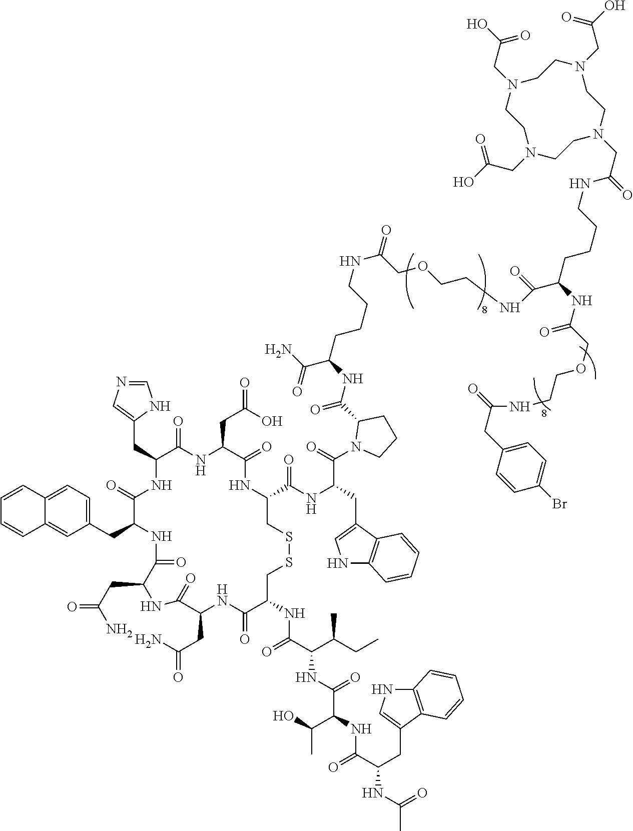

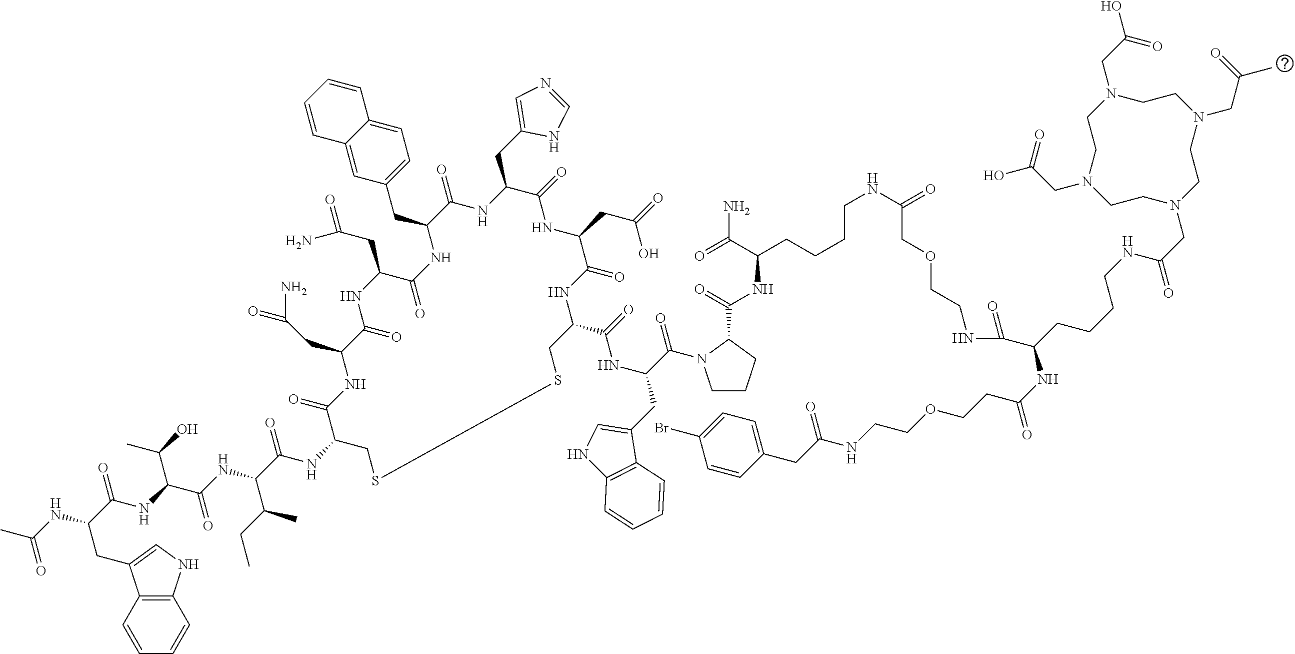 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)COCCNC(=O)[C@@H](CCCCNC(=O)CN2CCN(CC(C)=O)CCN(CC(=O)O)CCN(CC(=O)O)CC2)NC(=O)CCOCCNC(=O)Cc2ccc(Br)cc2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

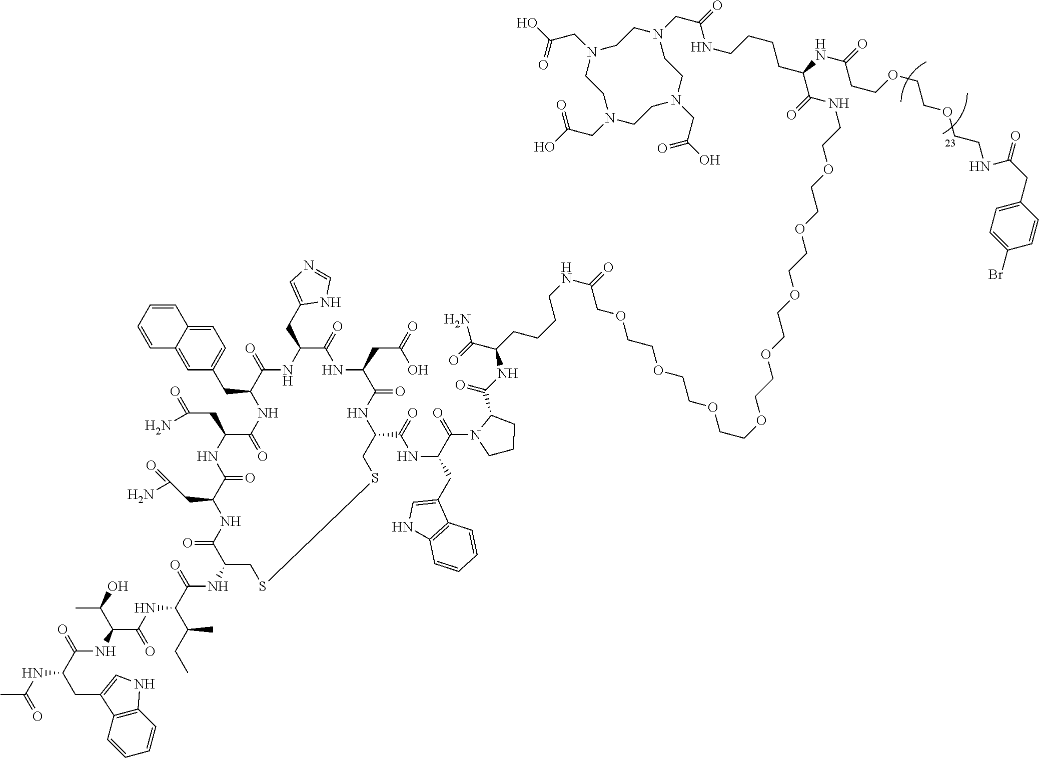

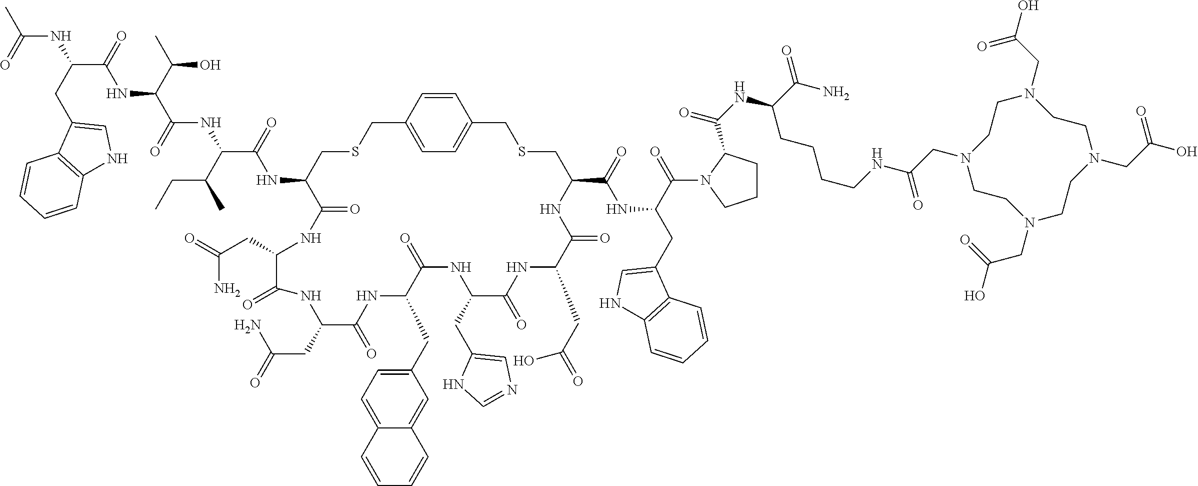 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCc2ccc(cc2)CSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

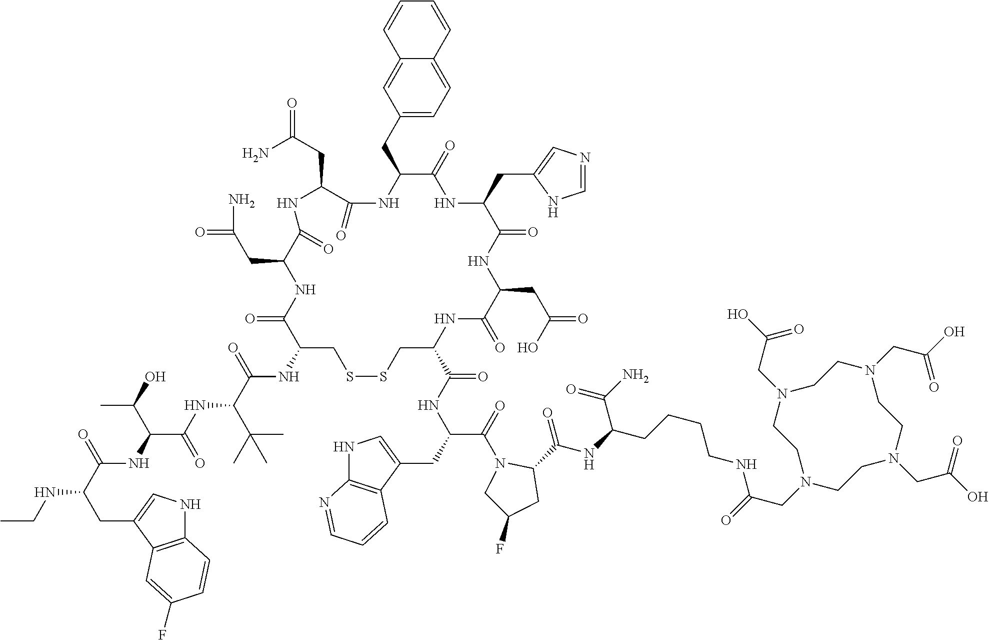 [SMILES: CCN[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C(C)(C)C)[C@@H](C)O]

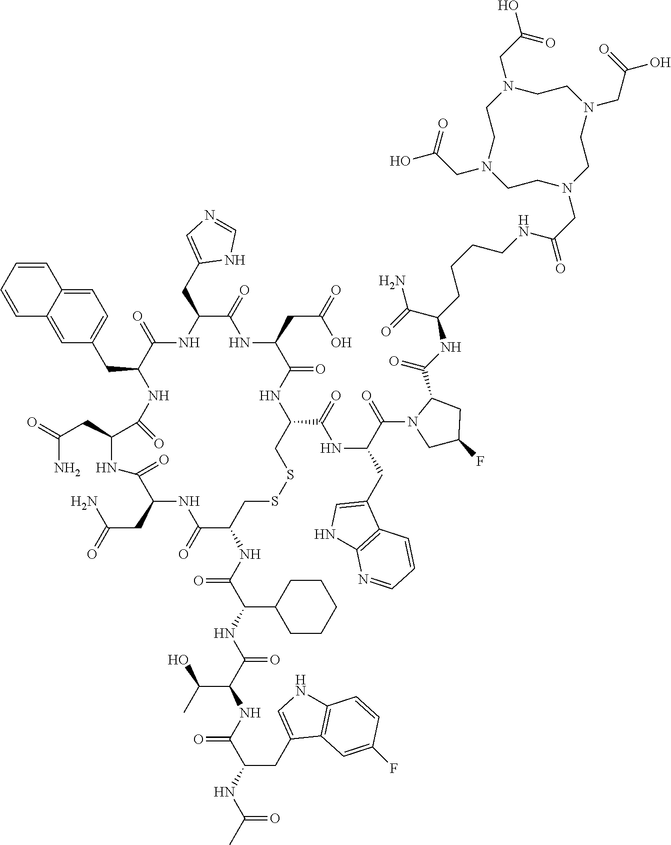 [SMILES: CC(=O)N[C@@H](Cc1c[nH]c2ccc(F)cc12)C(=O)N[C@H](C(=O)N[C@H](C(=O)N[C@H]1CSSC[C@@H](C(=O)N[C@@H](Cc2c[nH]c3ncccc23)C(=O)N2C[C@H](F)C[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)C1CCCCC1)[C@@H](C)O]

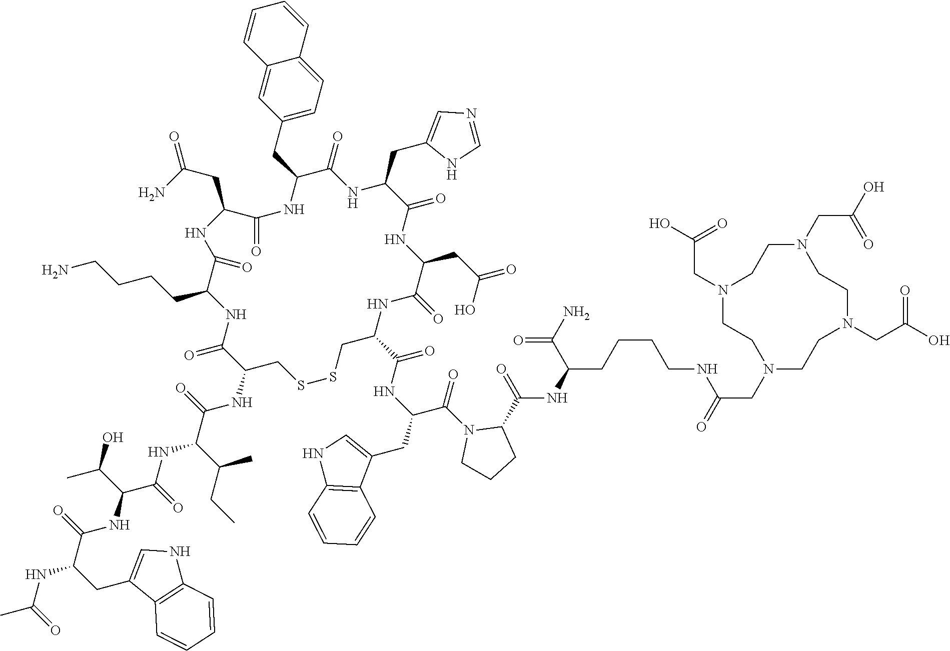 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CCCCN)NC1=O]

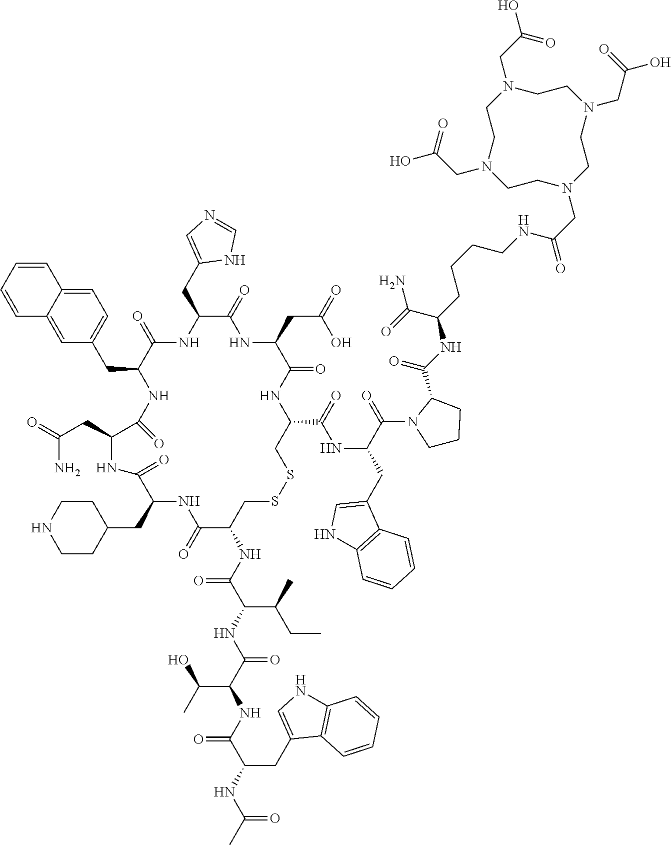 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O]

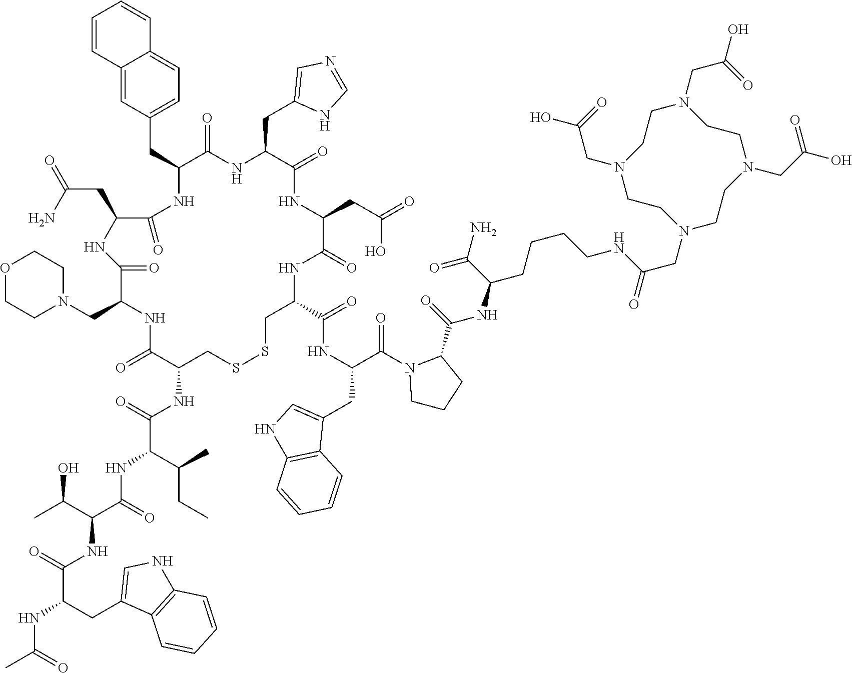 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CN2CCOCC2)NC1=O]

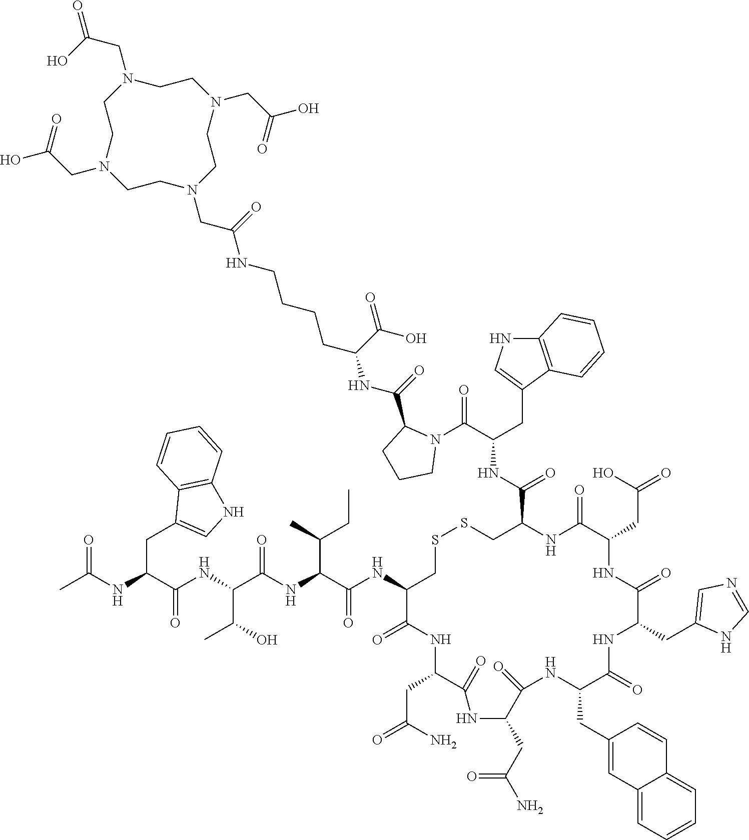 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(=O)O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

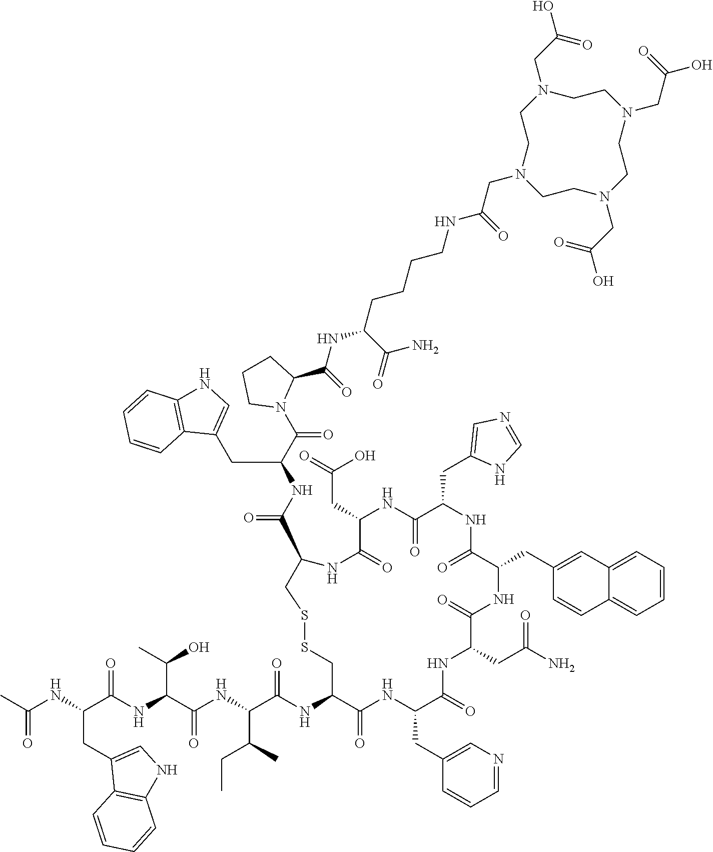 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O]

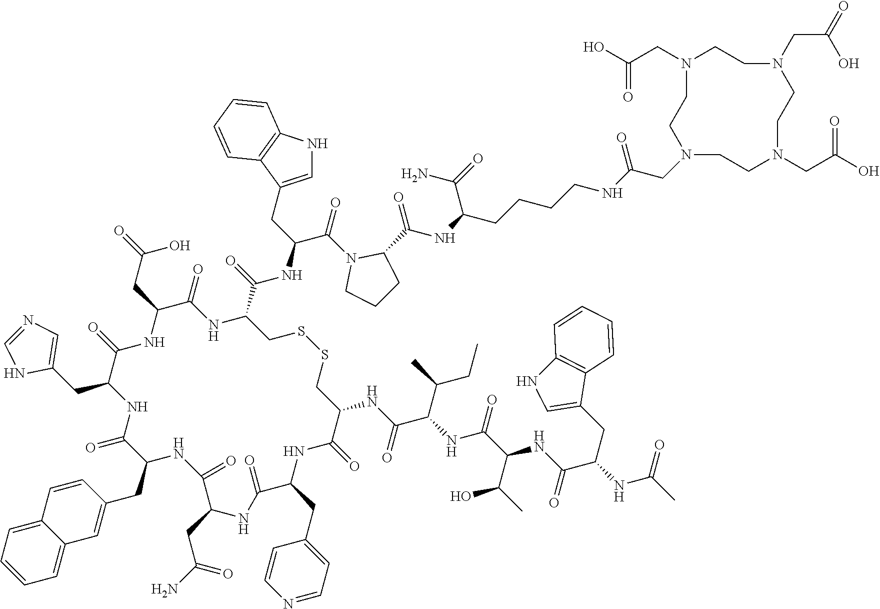 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2ccncc2)NC1=O]

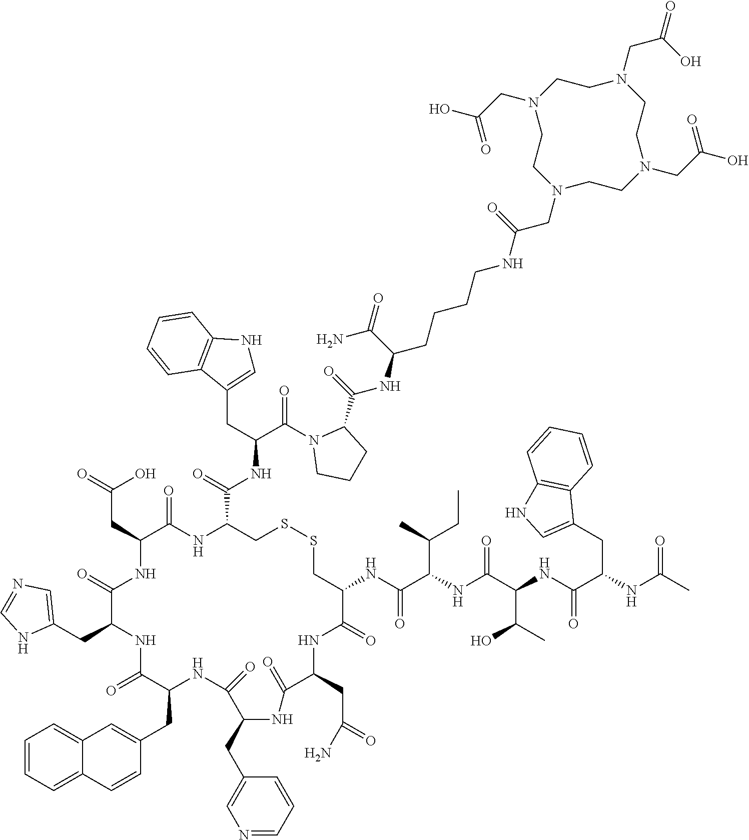 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](Cc2cccnc2)NC(=O)[C@H](CC(N)=O)NC1=O]

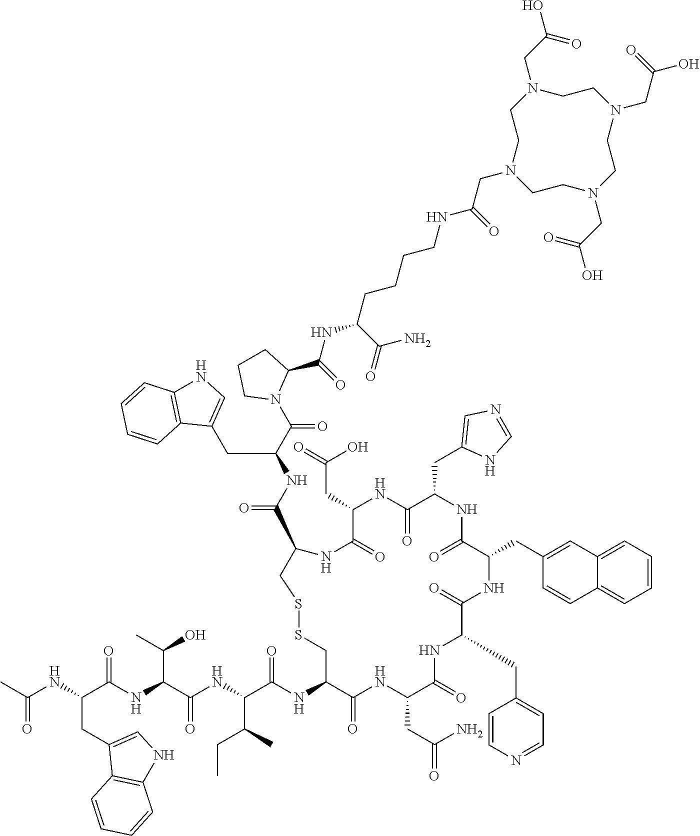 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](Cc2ccncc2)NC(=O)[C@H](CC(N)=O)NC1=O]

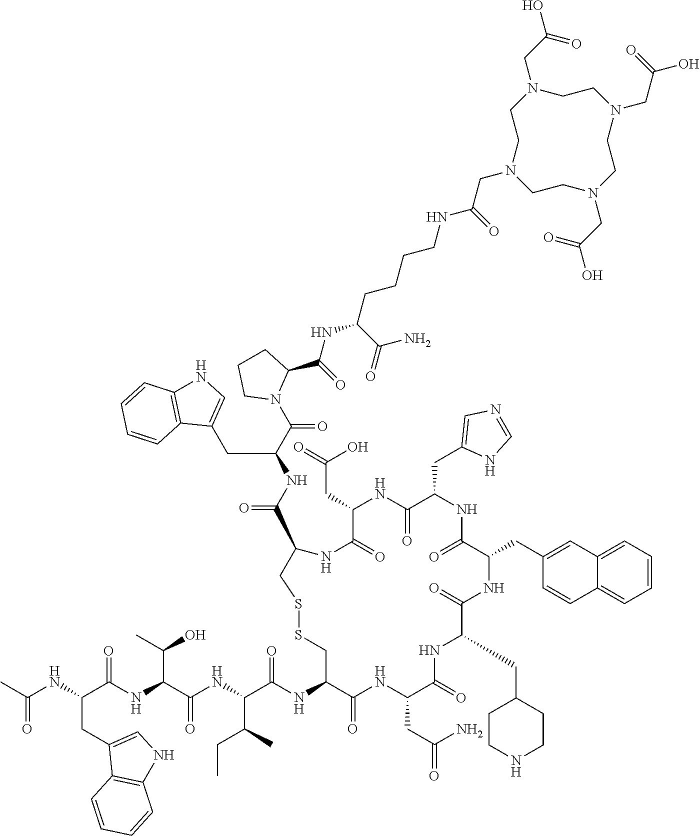 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC2CCNCC2)NC(=O)[C@H](CC(N)=O)NC1=O]

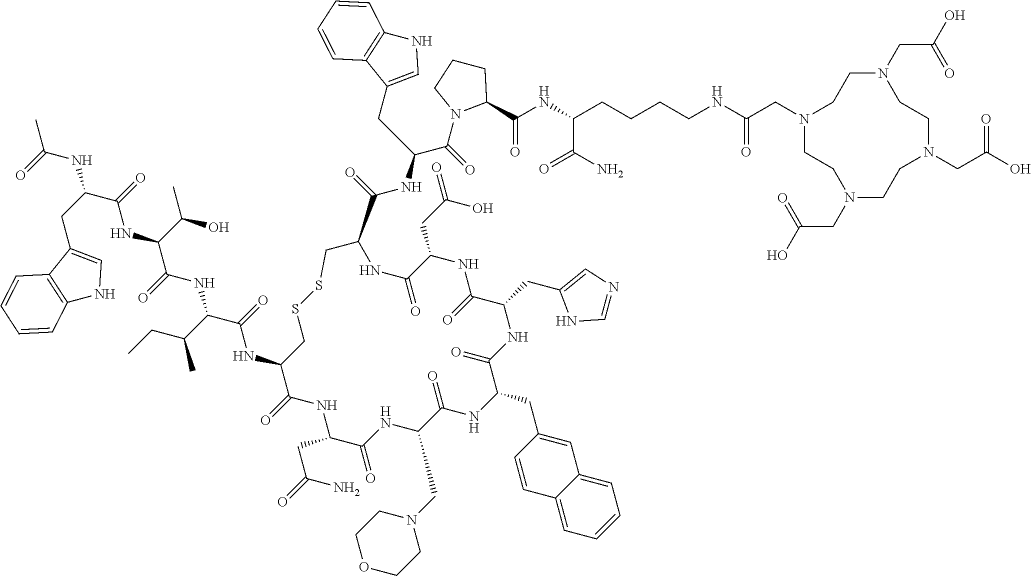 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CN2CCOCC2)NC(=O)[C@H](CC(N)=O)NC1=O]

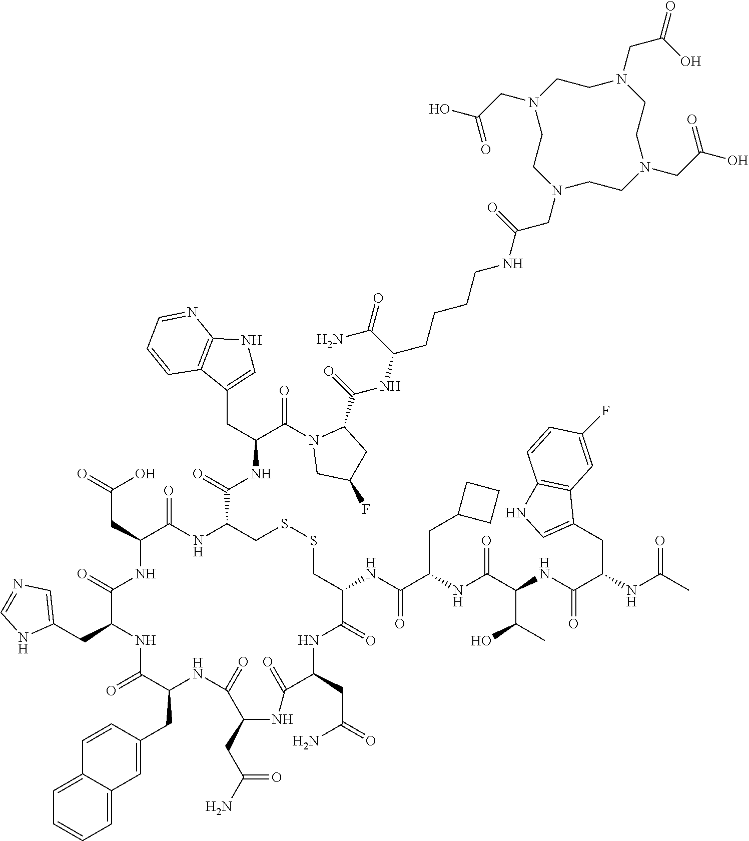 [SMILES: CC(=O)N[C@@H](Cc1c[nH]c2ccc(F)cc12)C(=O)N[C@H](C(=O)N[C@@H](CC1CCC1)C(=O)N[C@H]1CSSC[C@@H](C(=O)N[C@@H](Cc2c[nH]c3ncccc23)C(=O)N2C[C@H](F)C[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O)[C@@H](C)O]

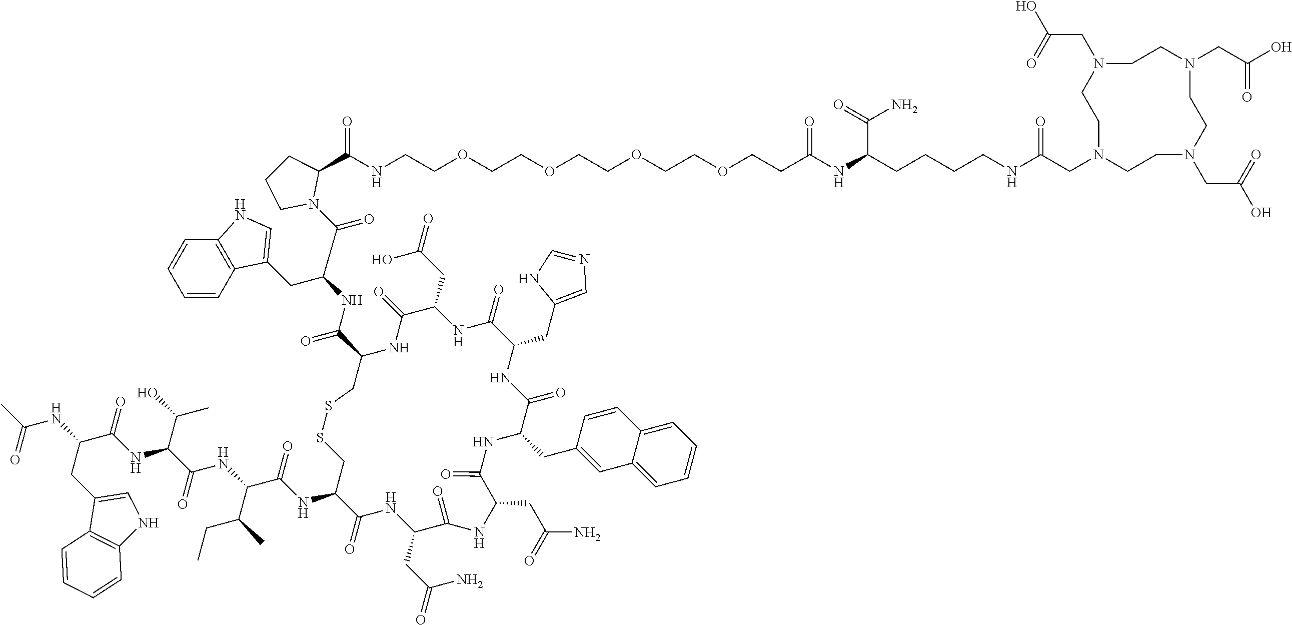 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCCOCCOCCOCCOCCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

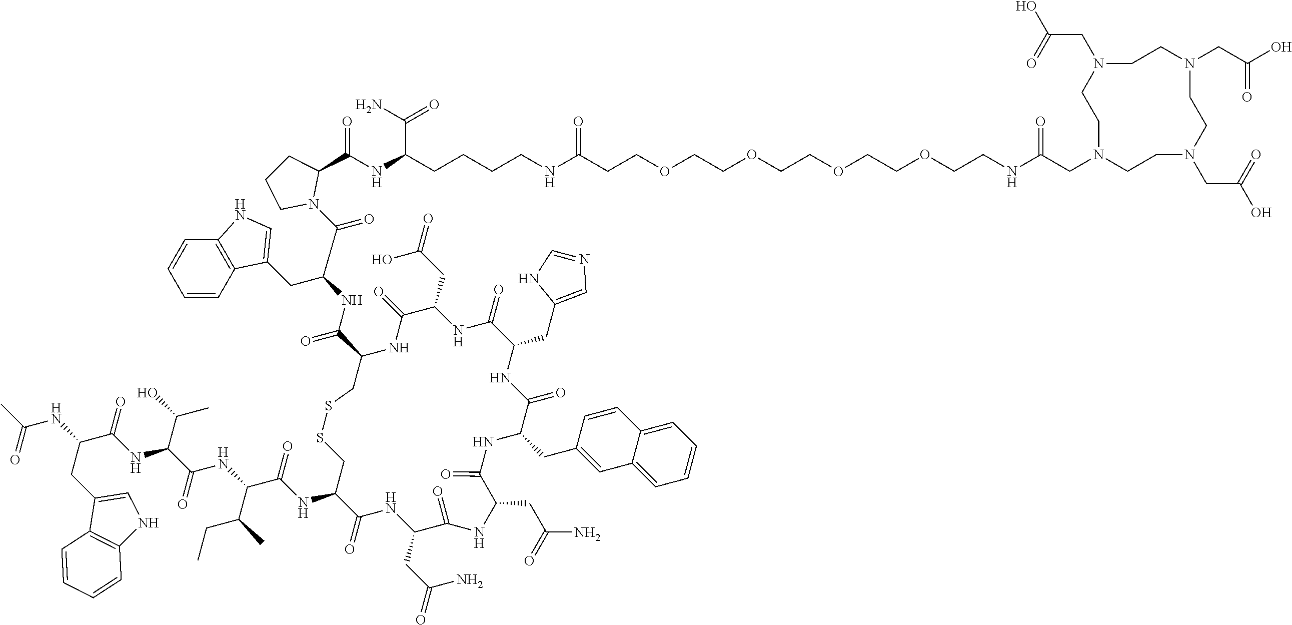 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CCOCCOCCOCCOCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

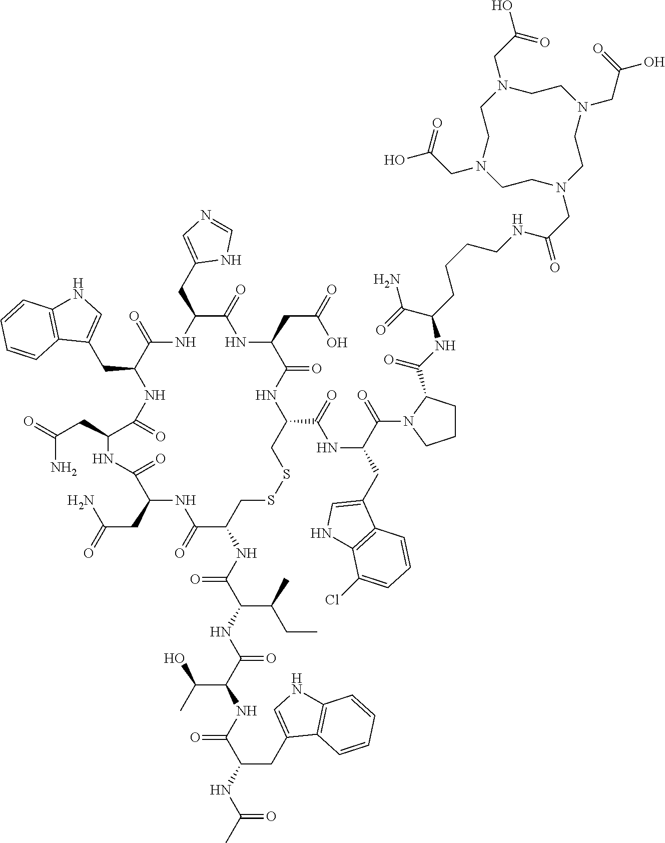 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3c(Cl)cccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

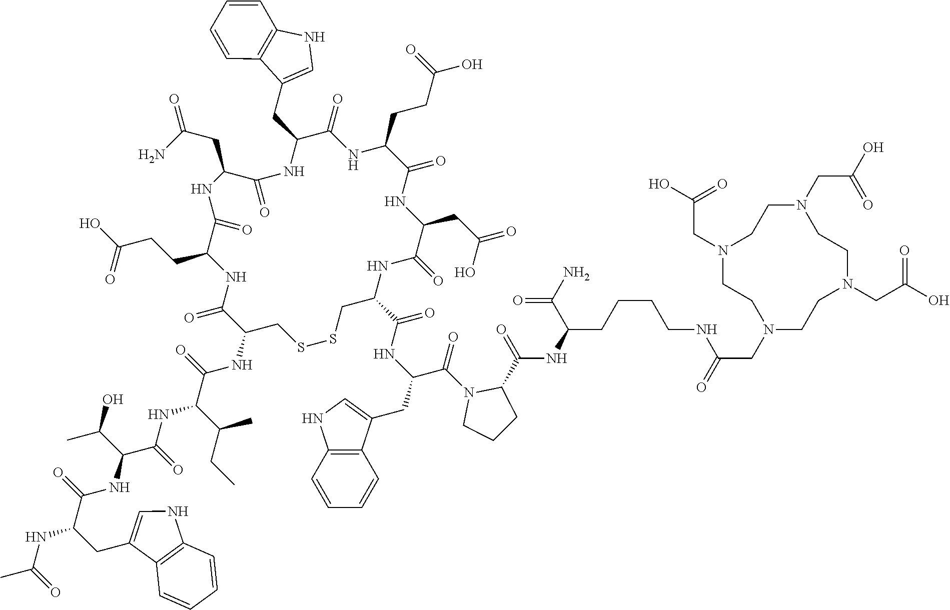 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CC(N)=O)NC(=O)[C@H](CCC(=O)O)NC1=O]

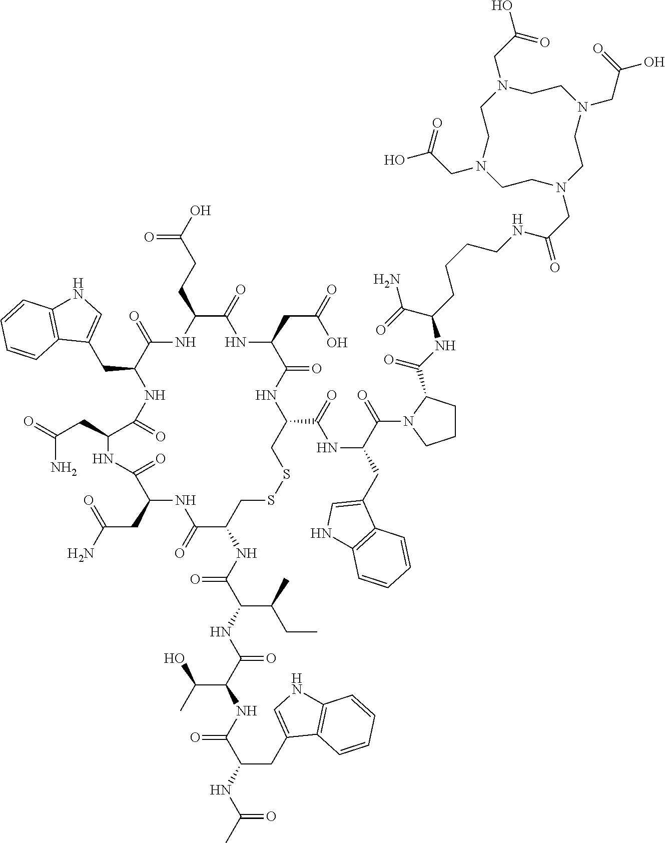 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CC(N)=O)NC(=O)[C@H](CC(N)=O)NC1=O]

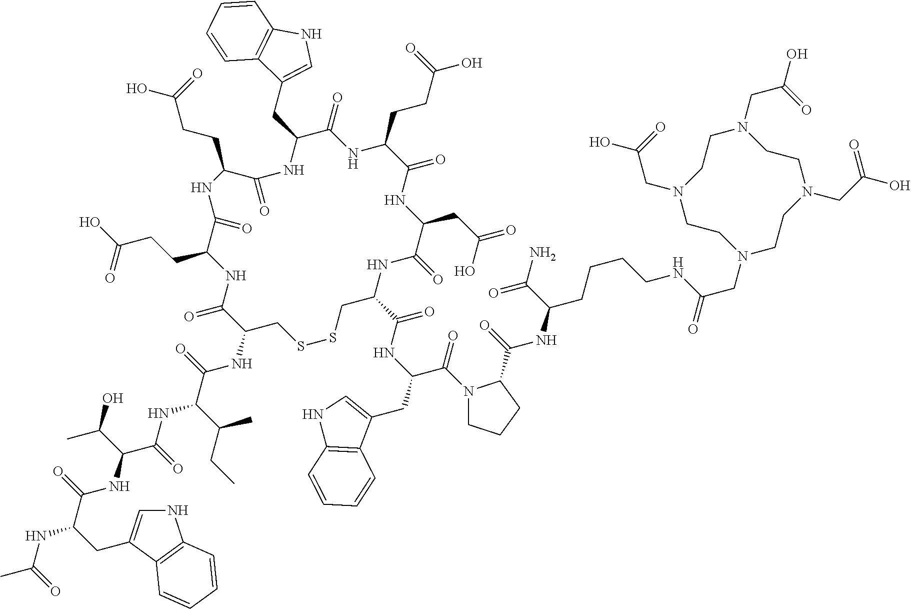 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CCC(=O)O)NC(=O)[C@H](CCC(=O)O)NC1=O]

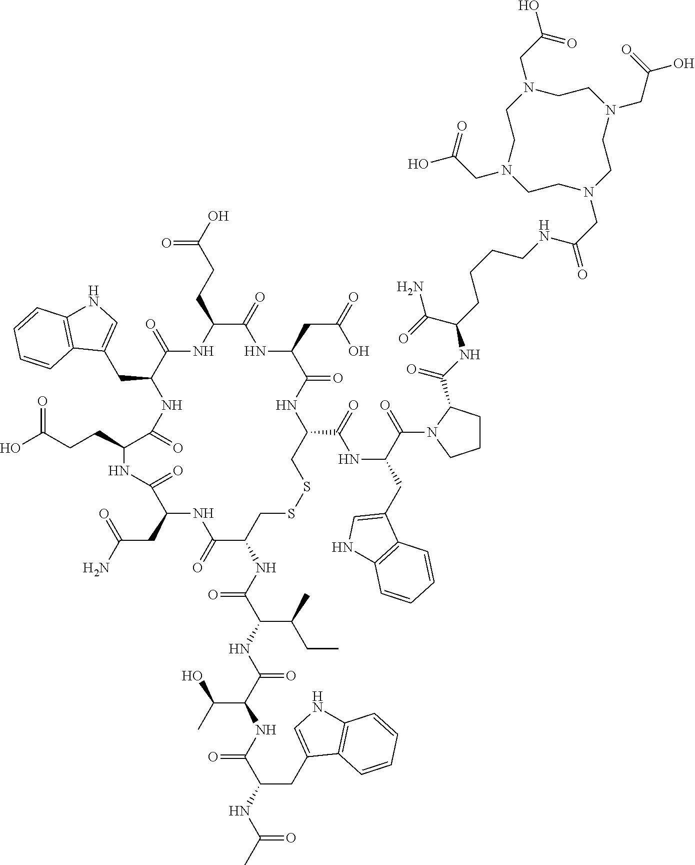 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCC(=O)O)NC(=O)[C@H](Cc2c[nH]c3ccccc23)NC(=O)[C@H](CCC(=O)O)NC(=O)[C@H](CC(N)=O)NC1=O]

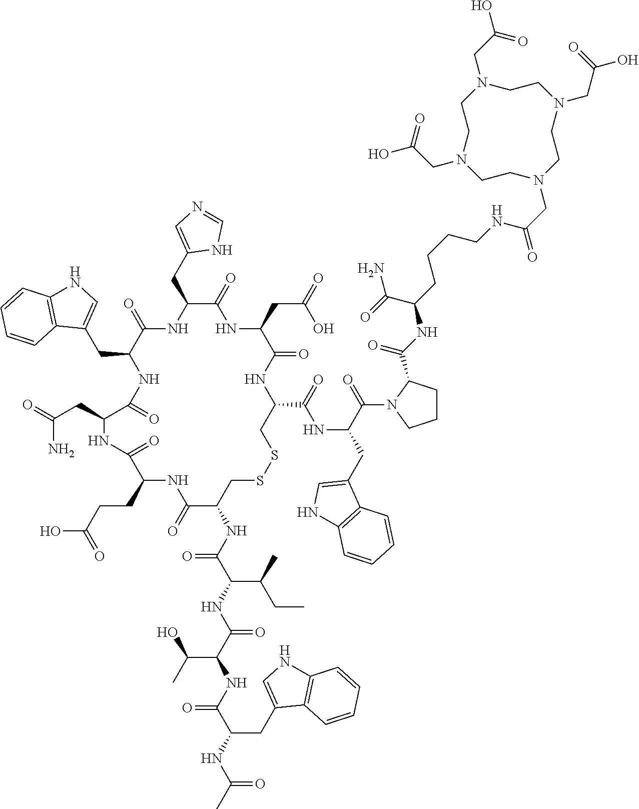 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CCC(=O)O)NC1=O]

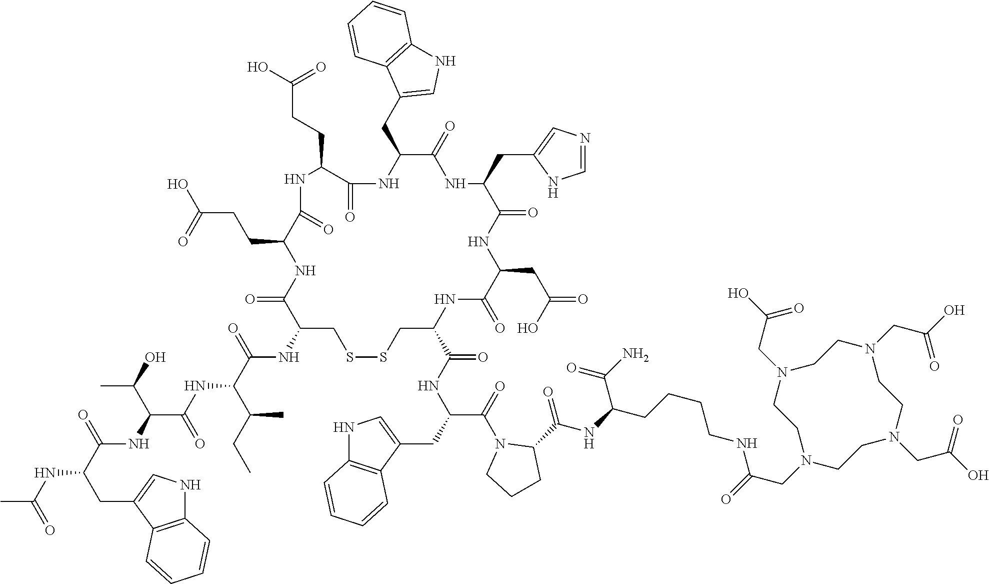 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CCC(=O)O)NC(=O)[C@H](CCC(=O)O)NC1=O]

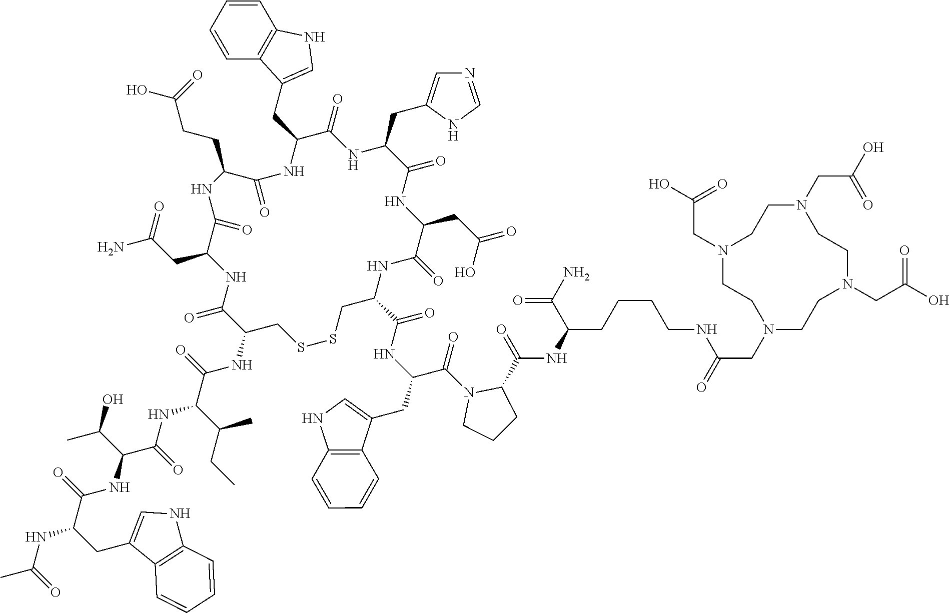 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CCC(=O)O)NC(=O)[C@H](CC(N)=O)NC1=O]

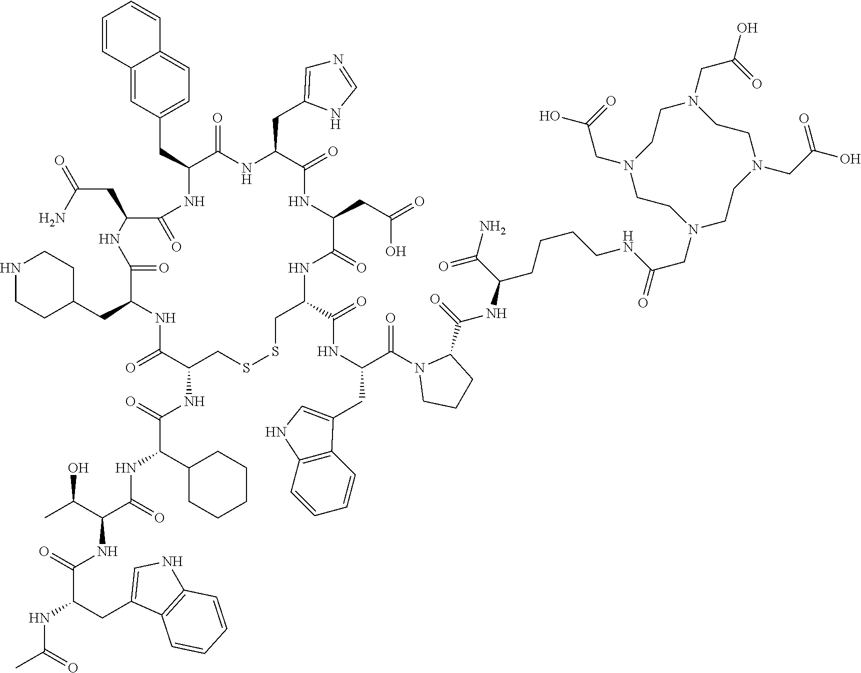 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O]

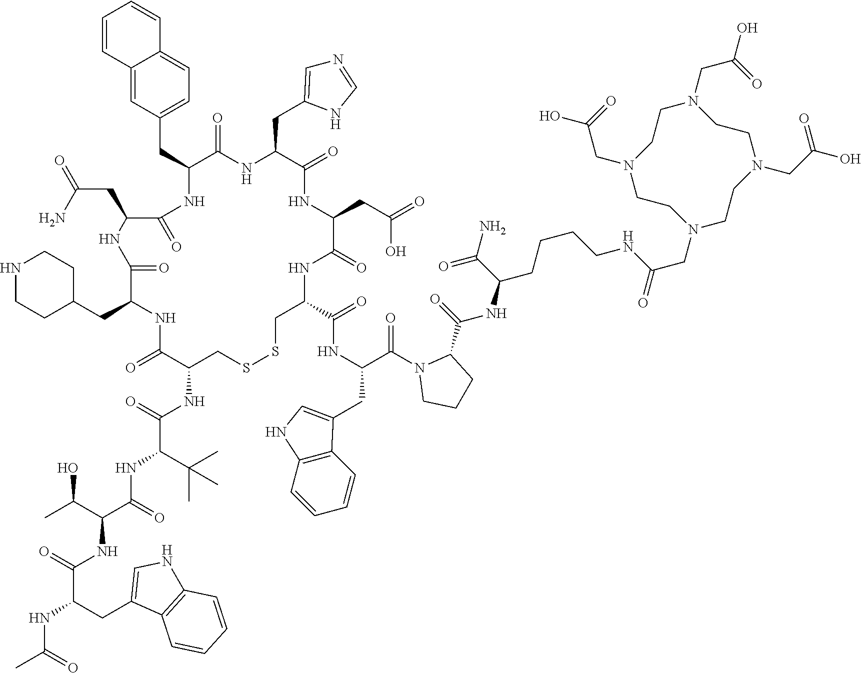 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C(C)(C)C)[C@@H](C)O]

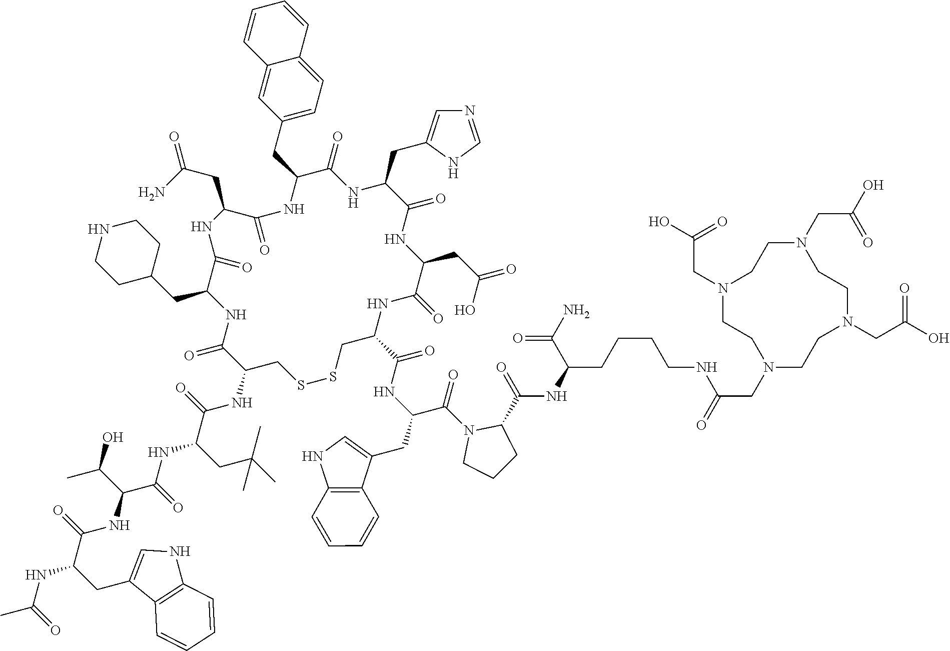 [SMILES: CC(=O)N[C@@H](Cc1c[nH]c2ccccc12)C(=O)N[C@H](C(=O)N[C@@H](CC(C)(C)C)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)[C@@H](C)O]

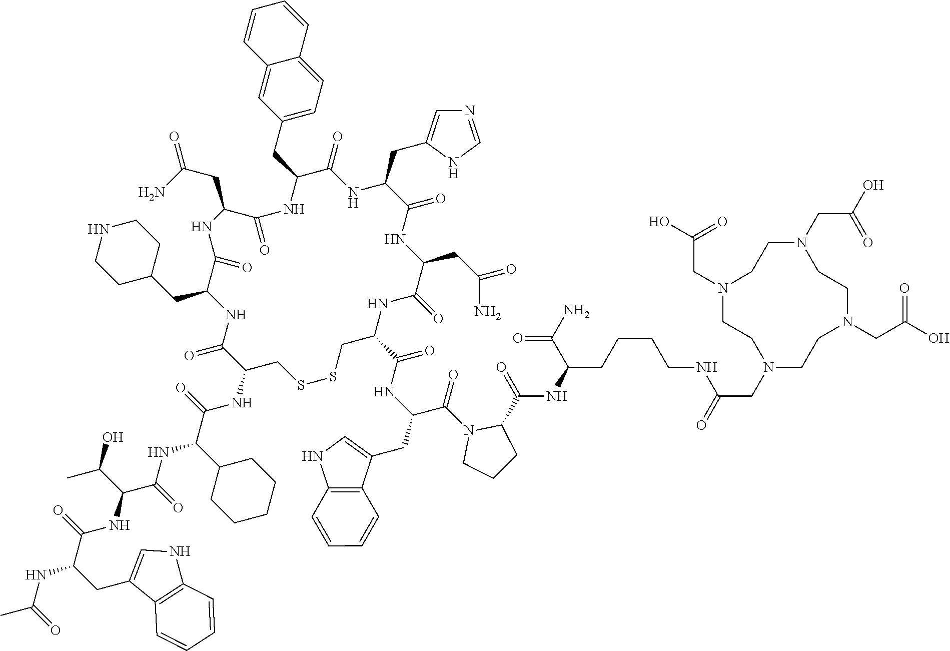 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(N)=O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O]

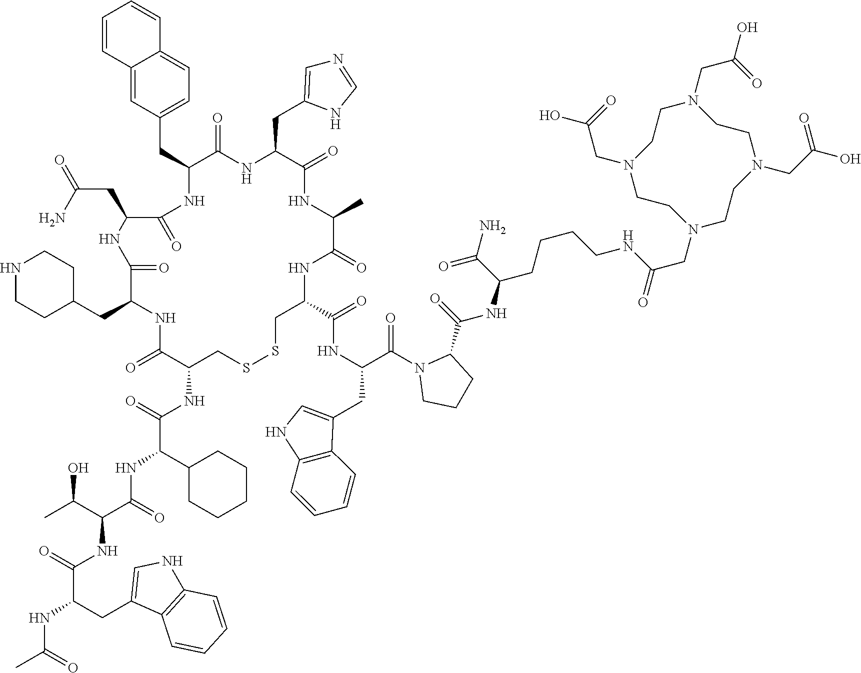 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](C)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O]

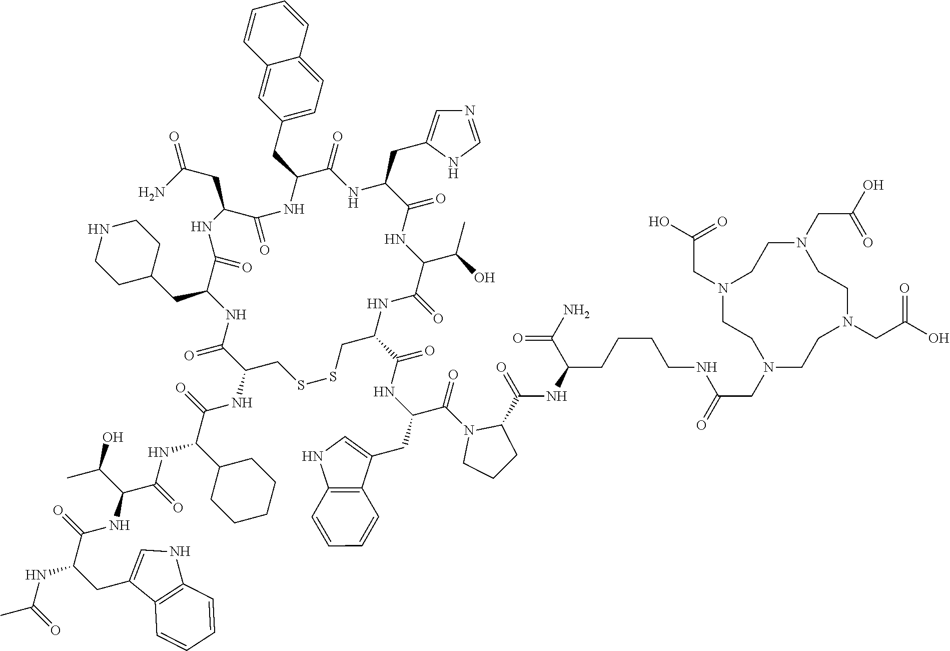 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)C([C@@H](C)O)NC(=O)[C@H](Cc2cnc[nH]2)NC(=O)[C@H](Cc2ccc3ccccc3c2)NC(=O)[C@H](CC(N)=O)NC(=O)[C@H](CC2CCNCC2)NC1=O)C1CCCCC1)[C@@H](C)O]

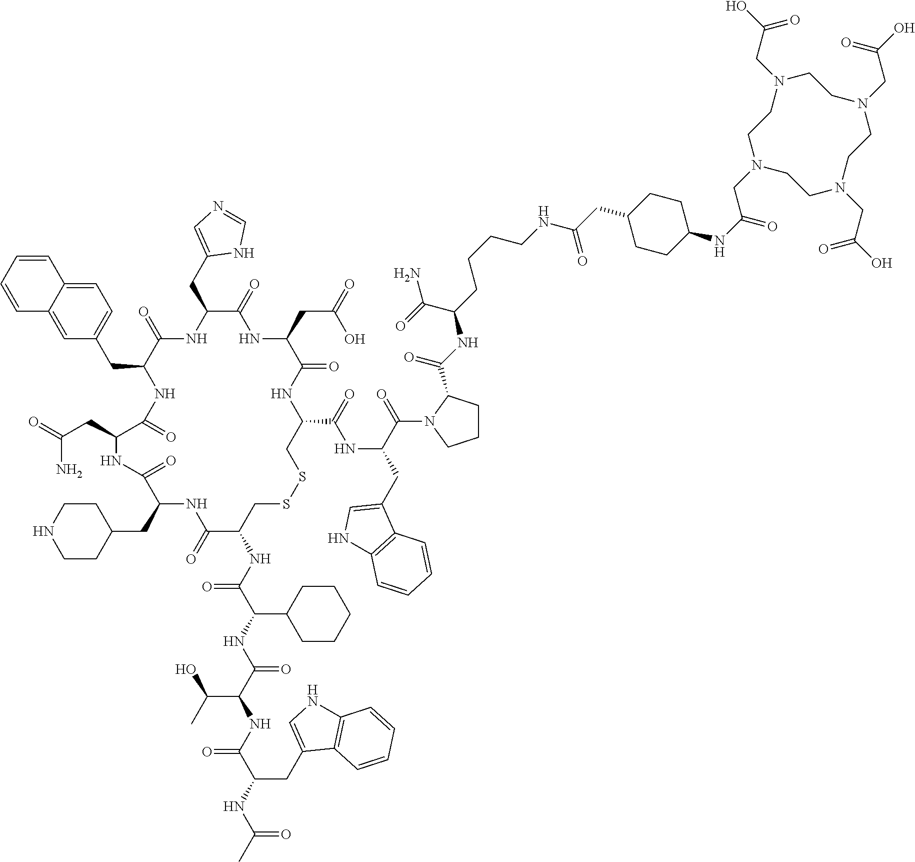

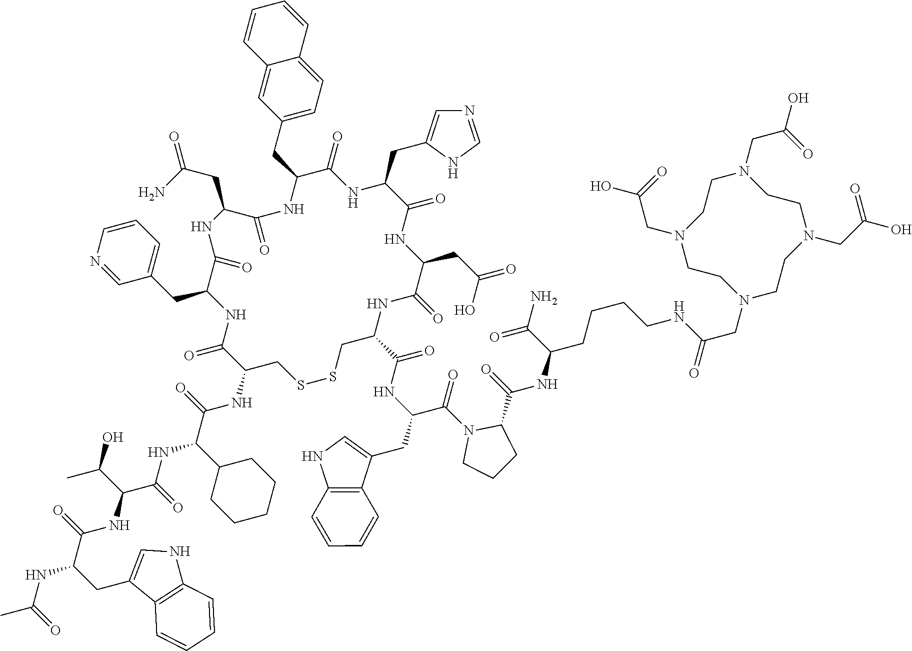 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O)C1CCCCC1)[C@@H](C)O]

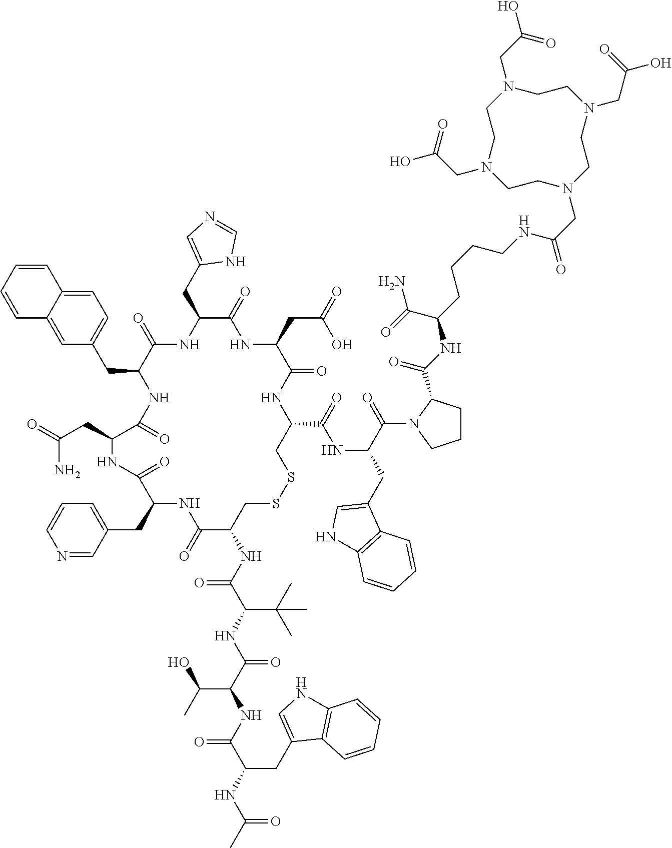 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O)C(C)(C)C)[C@@H](C)O]

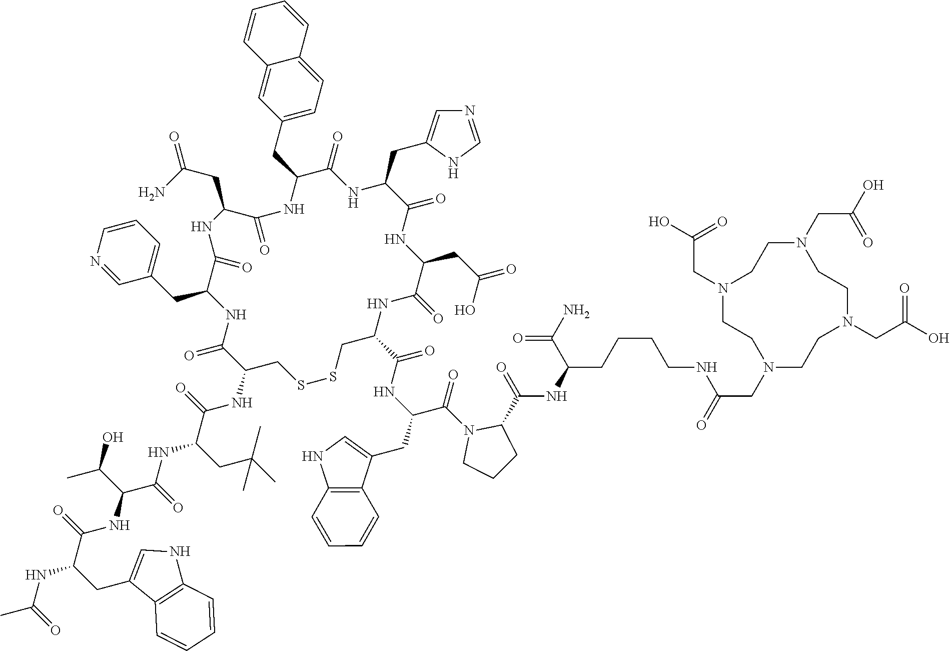 [SMILES: CC(=O)N[C@@H](Cc1c[nH]c2ccccc12)C(=O)N[C@H](C(=O)N[C@@H](CC(C)(C)C)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2cccnc2)NC1=O)[C@@H](C)O]

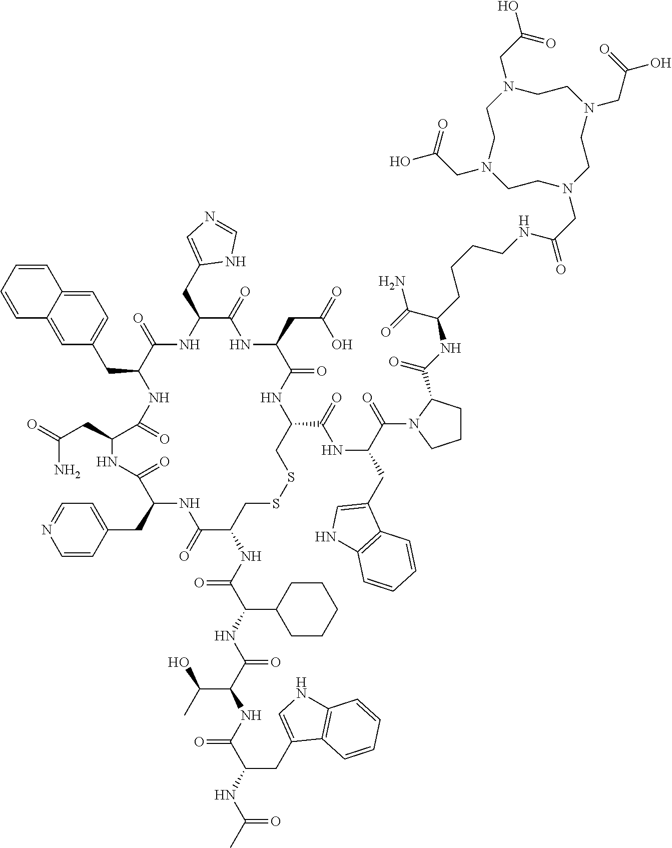 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2ccncc2)NC1=O)C1CCCCC1)[C@@H](C)O]

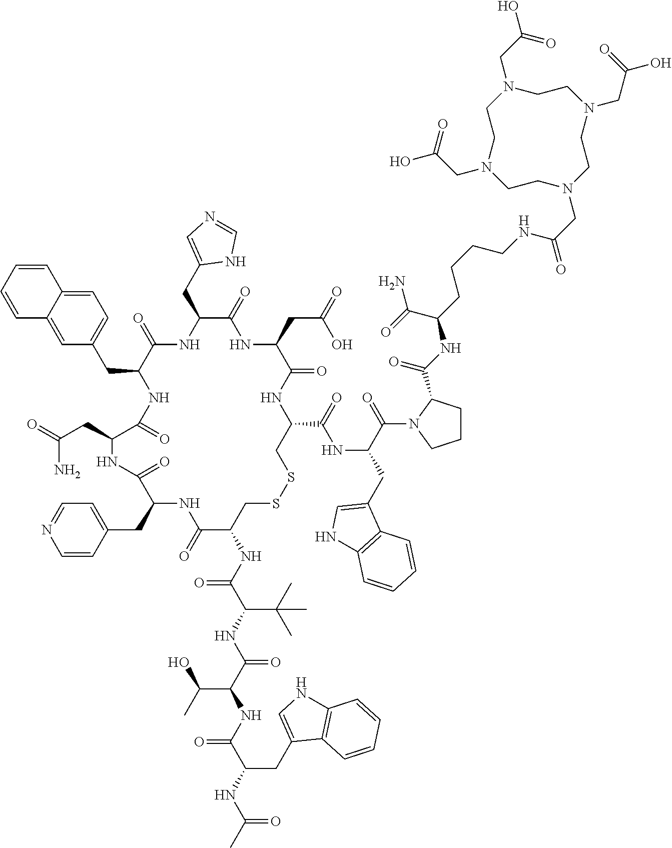 [SMILES: CC(=O)N[C@@H](Cc1c[nH]c2ccccc12)C(=O)N[C@H](C(=O)N[C@H](C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](Cc2ccncc2)NC1=O)C(C)(C)C)[C@@H](C)O]

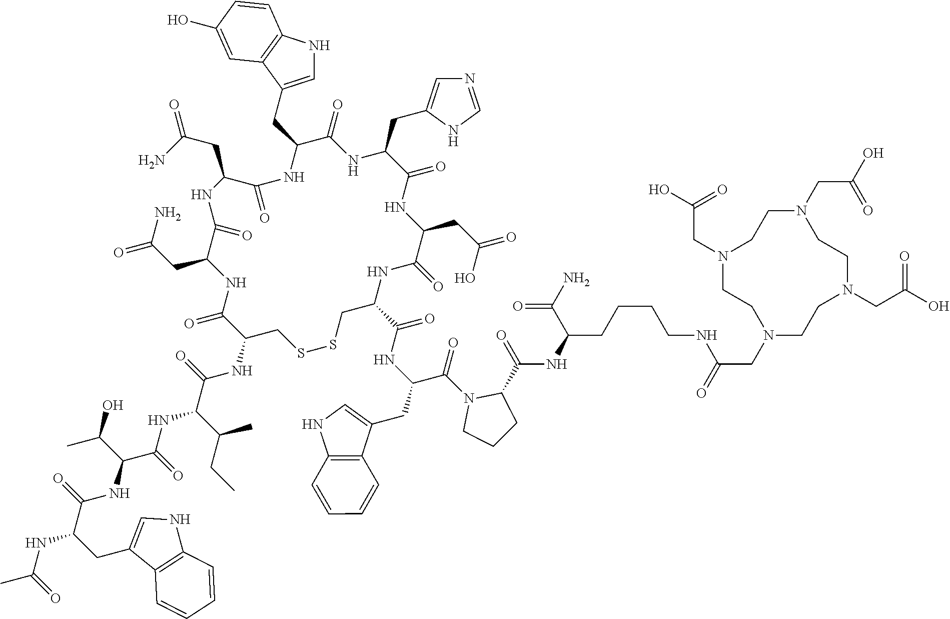 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccc(O)cc23)NC(=O)[C@H](CC(N)=O)NC(=O)[C@H](CC(N)=O)NC1=O]

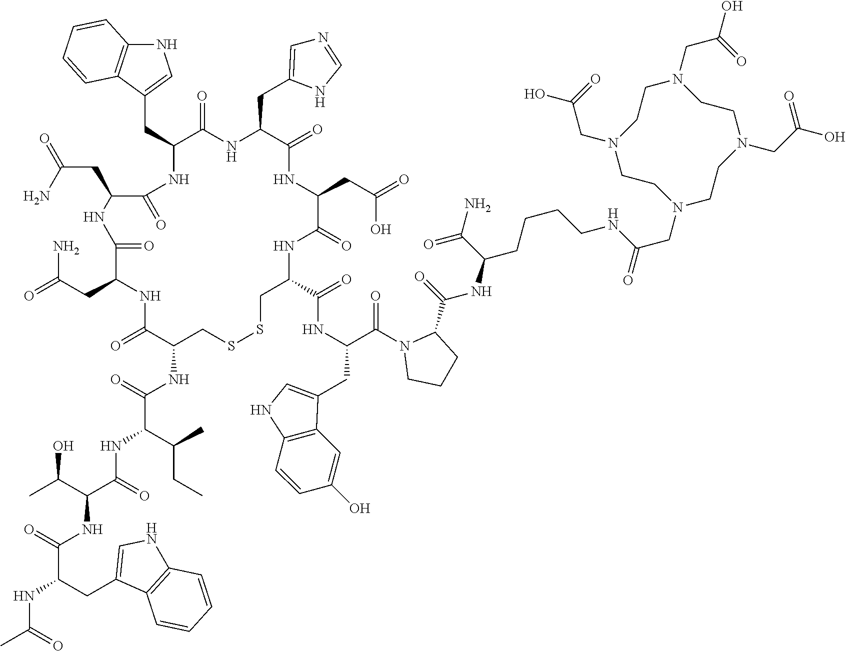 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccc(O)cc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

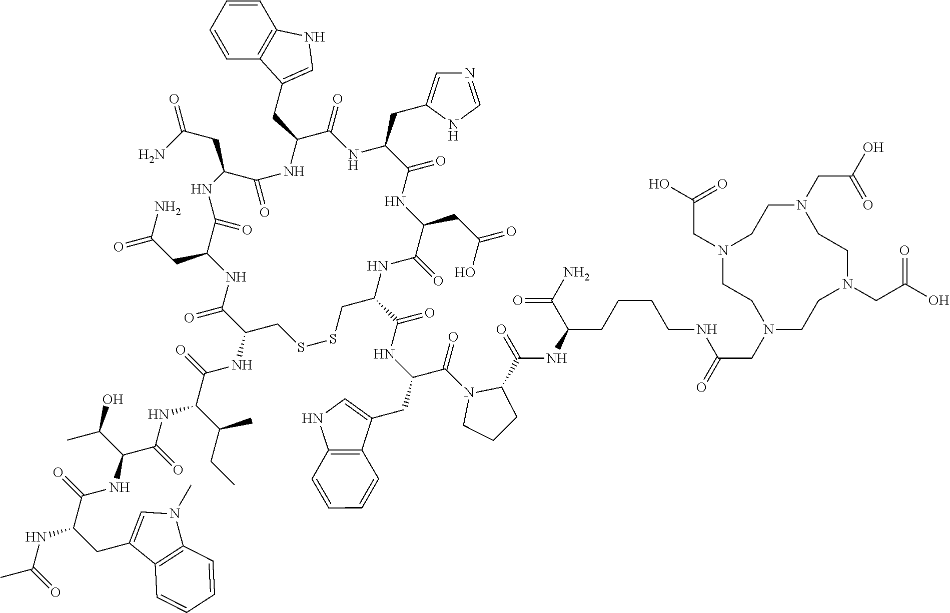 [SMILES: CC[C@H](C)[C@H](NC(=O)[C@@H](NC(=O)[C@H](Cc1cn(C)c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

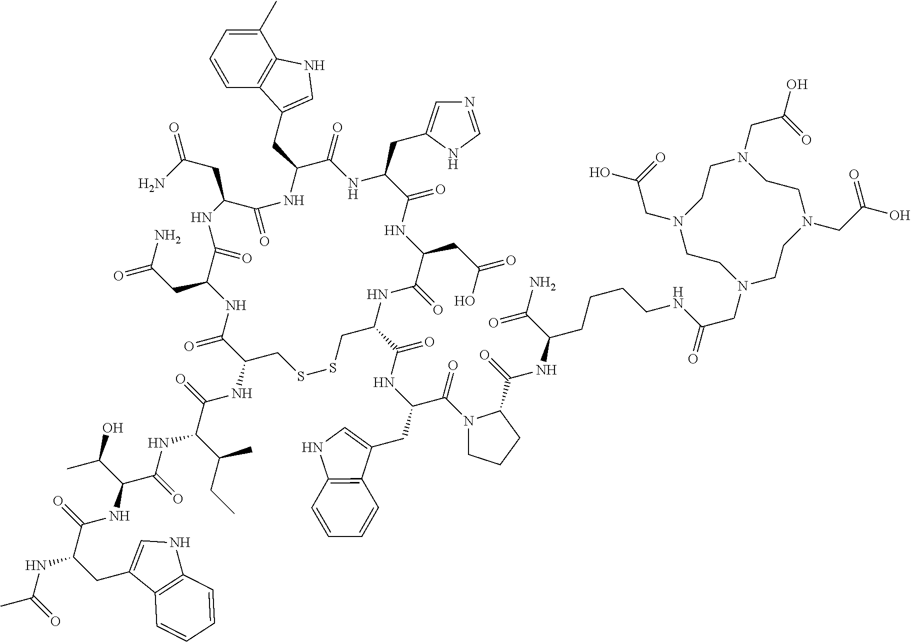 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3c(C)cccc23)NC(=O)[C@H](CC(N)=O)NC(=O)[C@H](CC(N)=O)NC1=O]

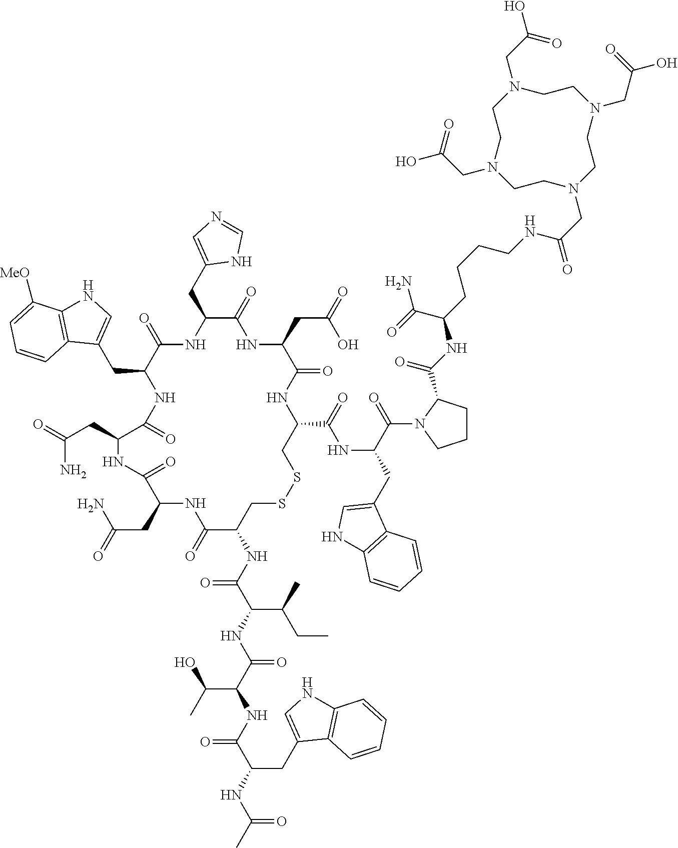 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3c(OC)cccc23)NC(=O)[C@H](CC(N)=O)NC(=O)[C@H](CC(N)=O)NC1=O]

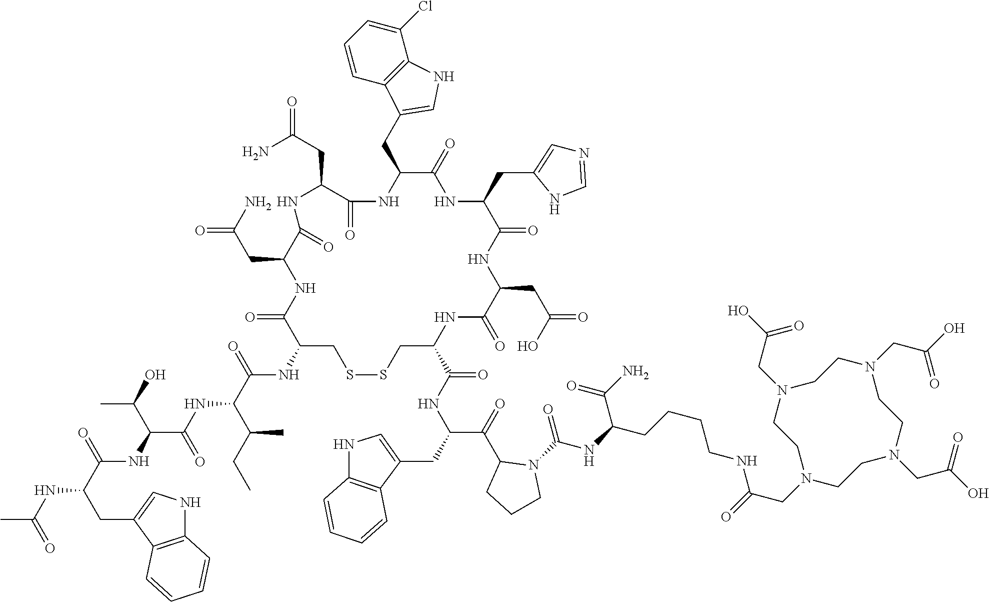

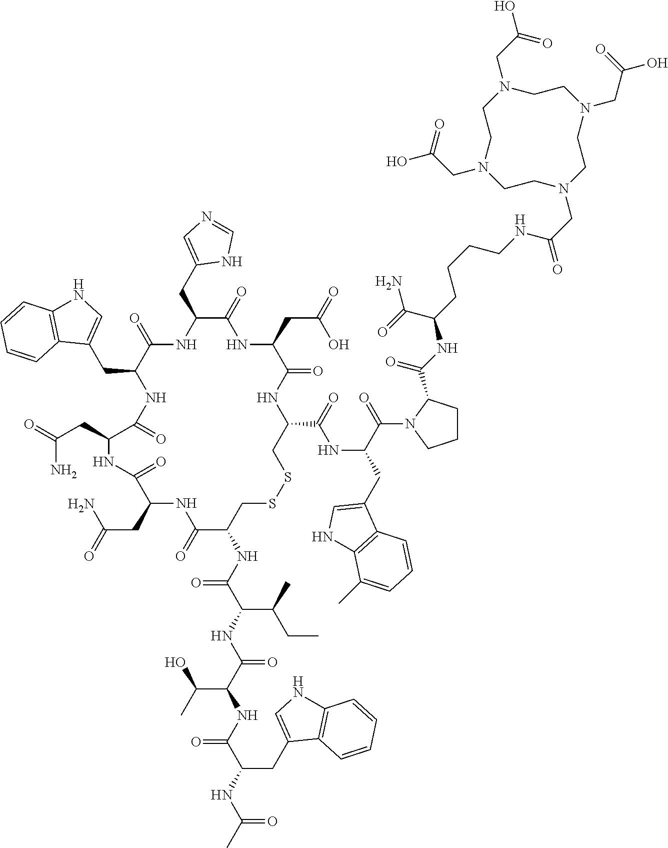 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3c(C)cccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

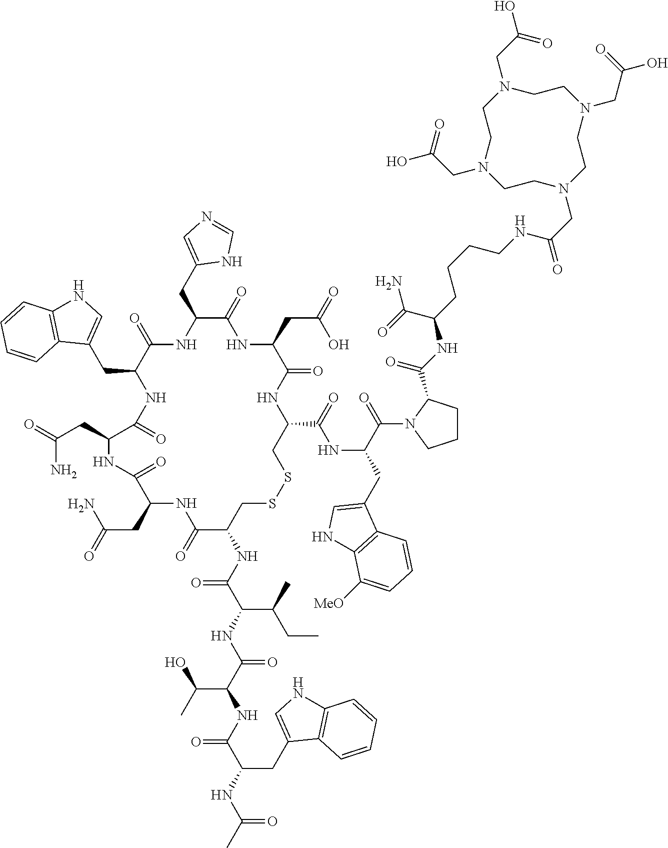 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3c(OC)cccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

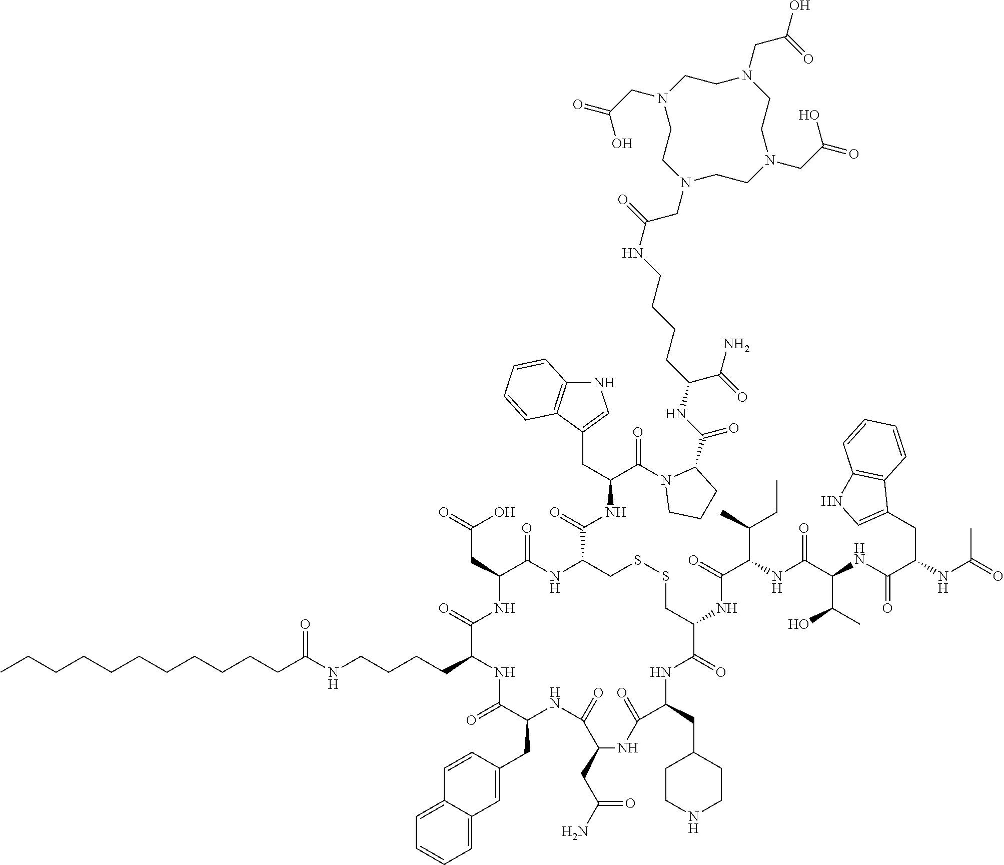 [SMILES: CCCCCCCCCCCC(=O)NCCCC[C@@H]1NC(=O)[C@H](Cc2ccc3ccccc3c2)NC(=O)[C@H](CC(N)=O)NC(=O)[C@H](CC2CCNCC2)NC(=O)[C@@H](NC(=O)[C@@H](NC(=O)[C@@H](NC(=O)[C@H](Cc2c[nH]c3ccccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O]

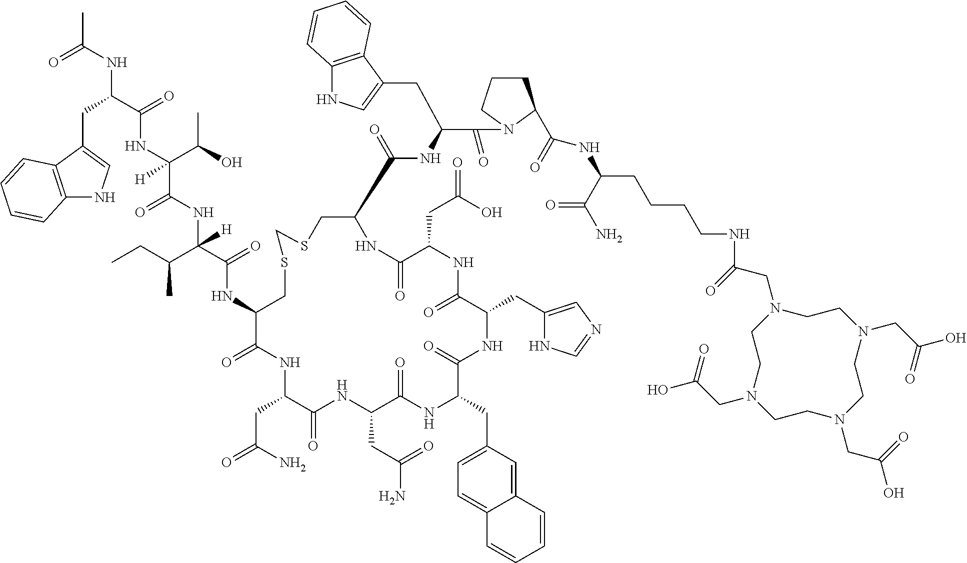 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSCSC[C@@H](C(=O)N[C@@H](Cc2c[nH]c3ccccc23)C(=O)N2CCC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

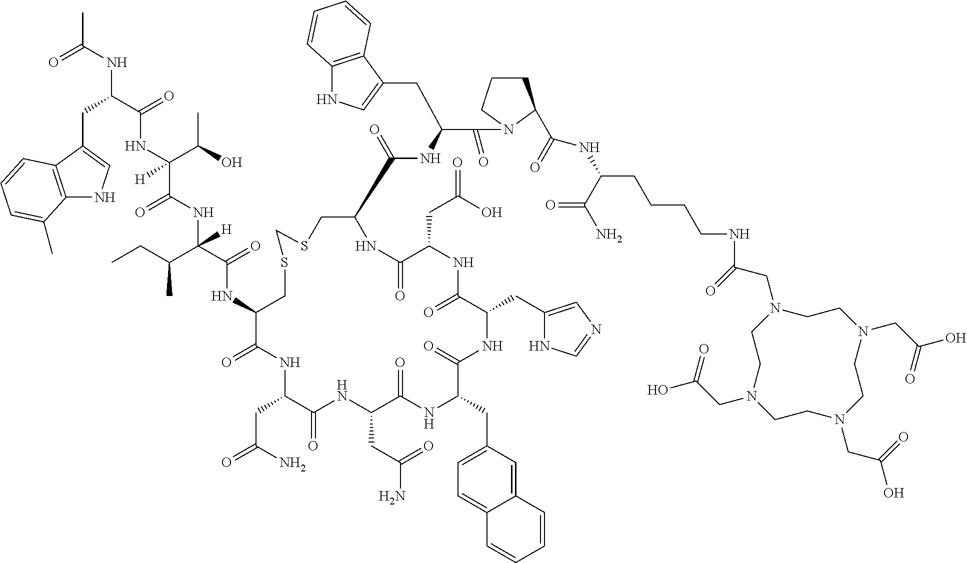 [SMILES: CC[C@H](C)[C@H](NC(=O)[C@@H](NC(=O)[C@H](Cc1c[nH]c2c(C)cccc12)NC(C)=O)[C@@H](C)O)C(=O)N[C@H]1CSCSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

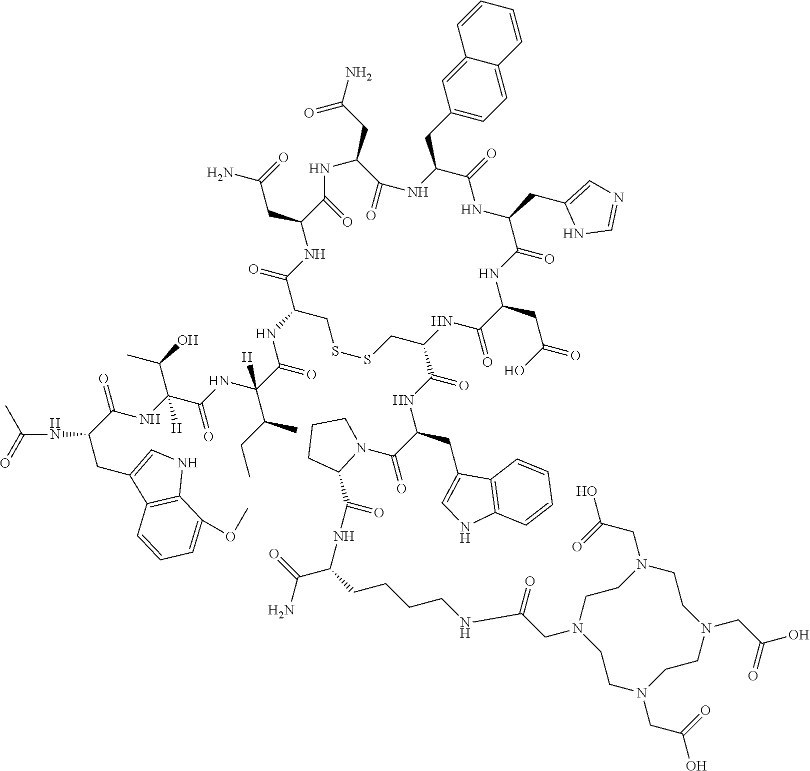 [SMILES: CC[C@H](C)[C@H](NC(=O)[C@@H](NC(=O)[C@H](Cc1c[nH]c2c(OC)cccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

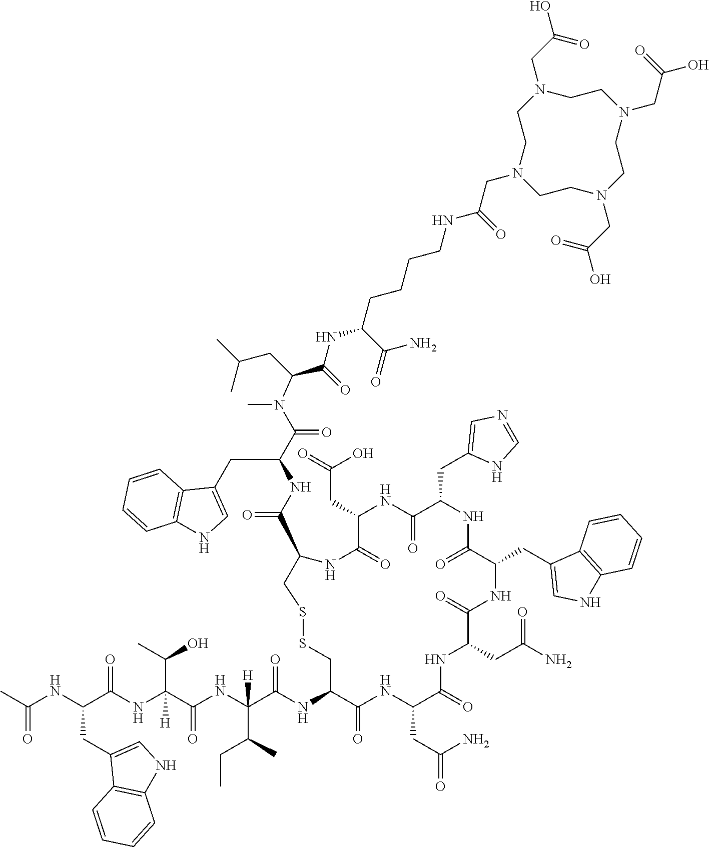 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)[C@@H](CC(C)C)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

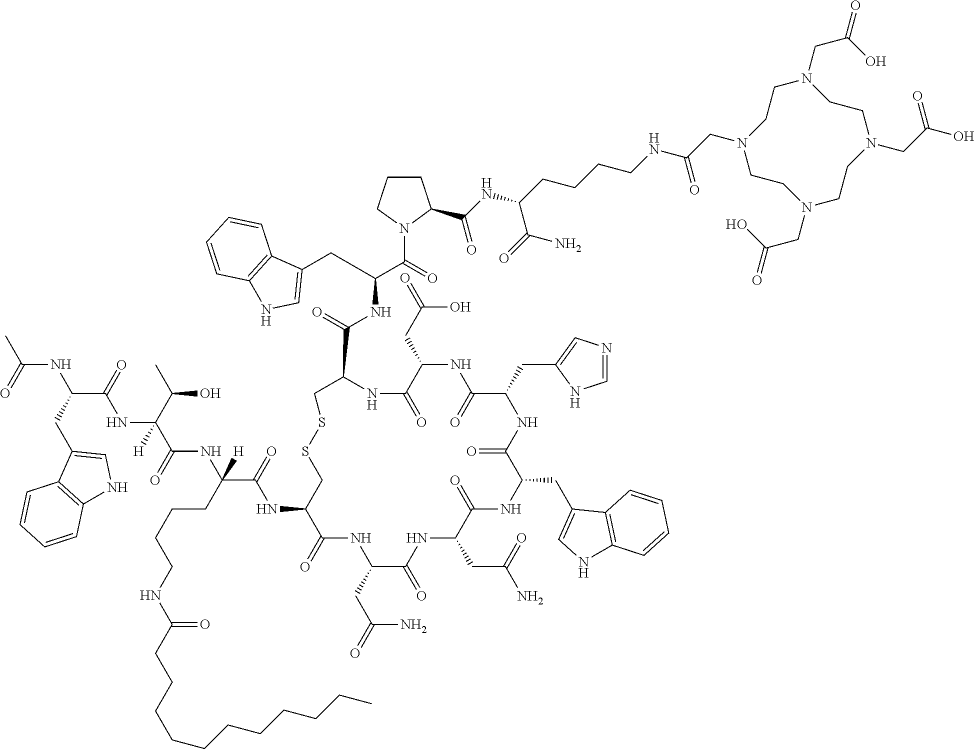 [SMILES: CCCCCCCCCCCC(=O)NCCCC[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

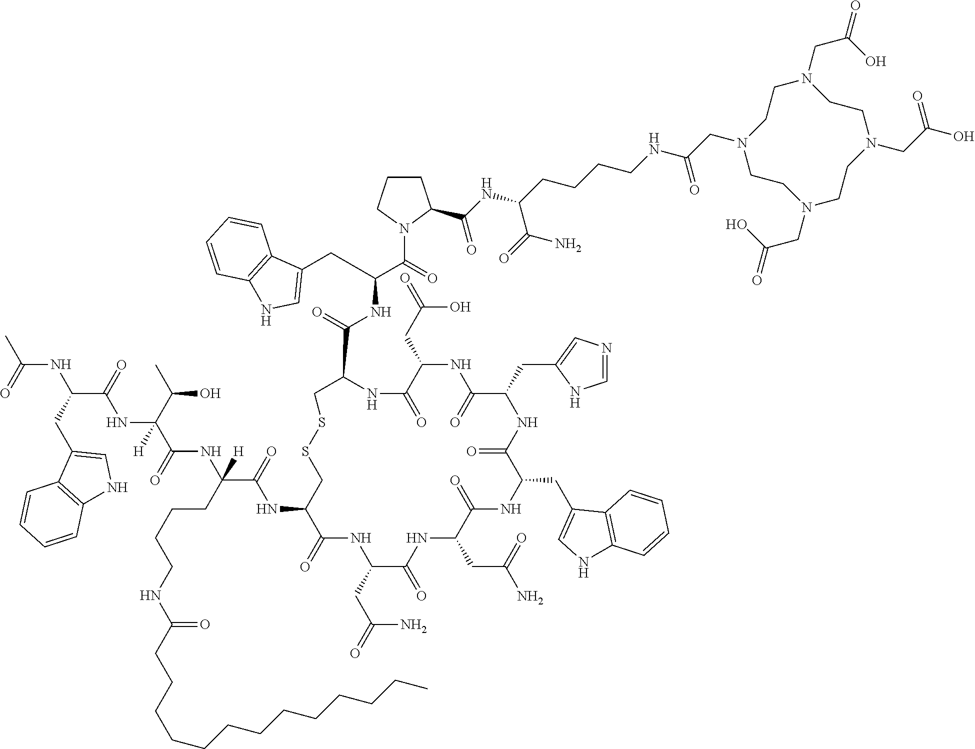 [SMILES: CCCCCCCCCCCCCC(=O)NCCCC[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

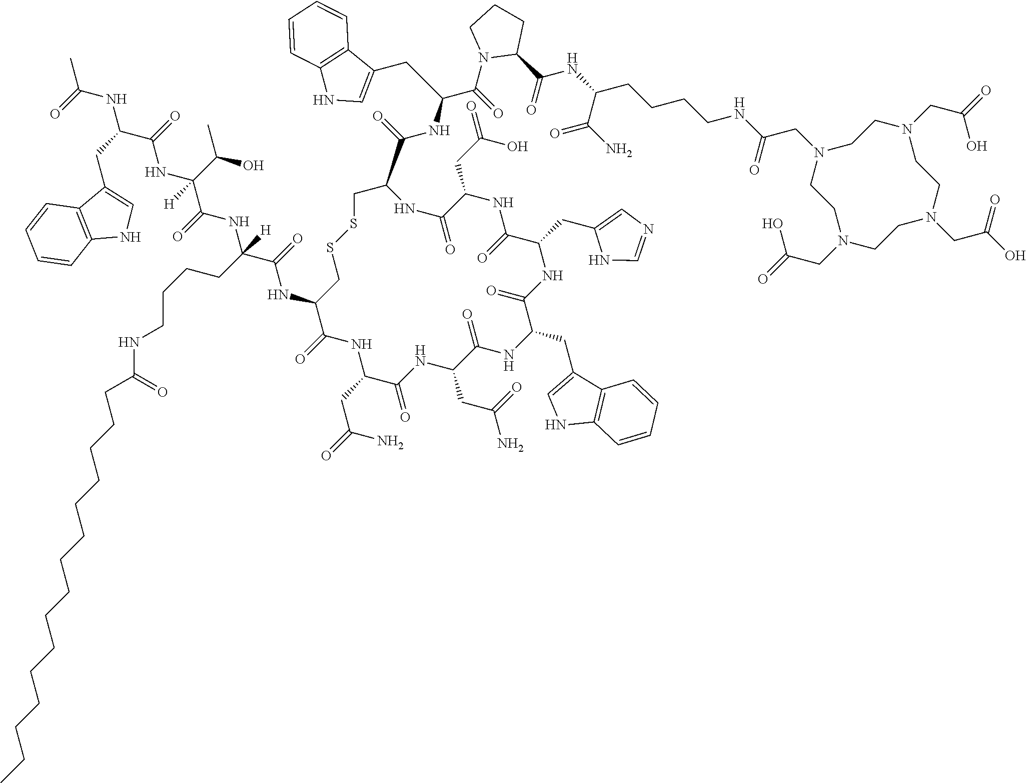 [SMILES: CCCCCCCCCCCCCCCC(=O)NCCCC[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

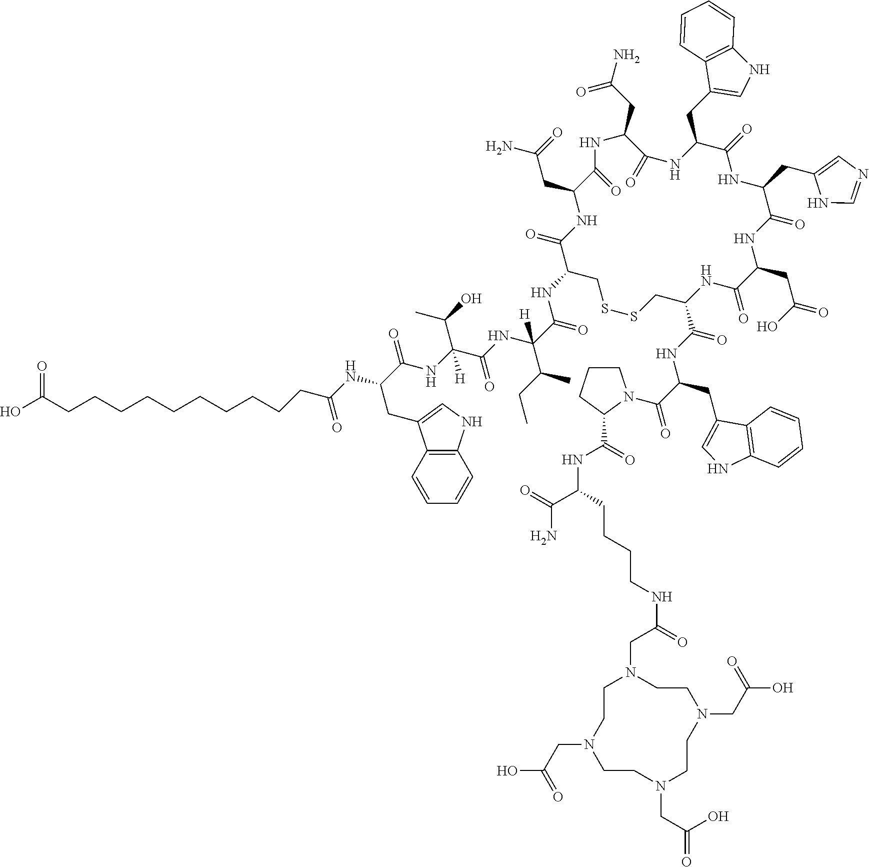 [SMILES: CC[C@H](C)[C@H](NC(=O)[C@@H](NC(=O)[C@H](Cc1c[nH]c2ccccc12)NC(=O)CCCCCCCCCCC(=O)O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

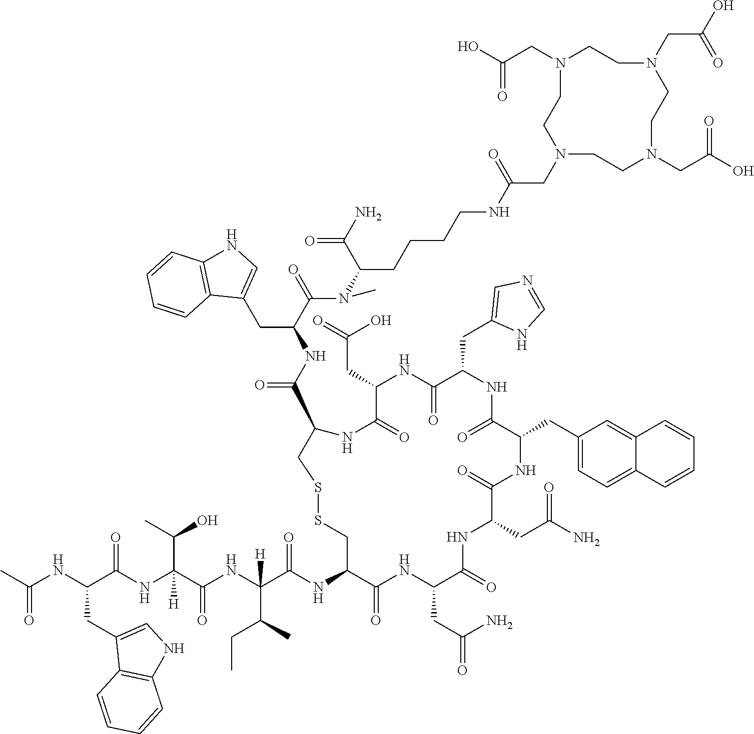 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N(C)[C@@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

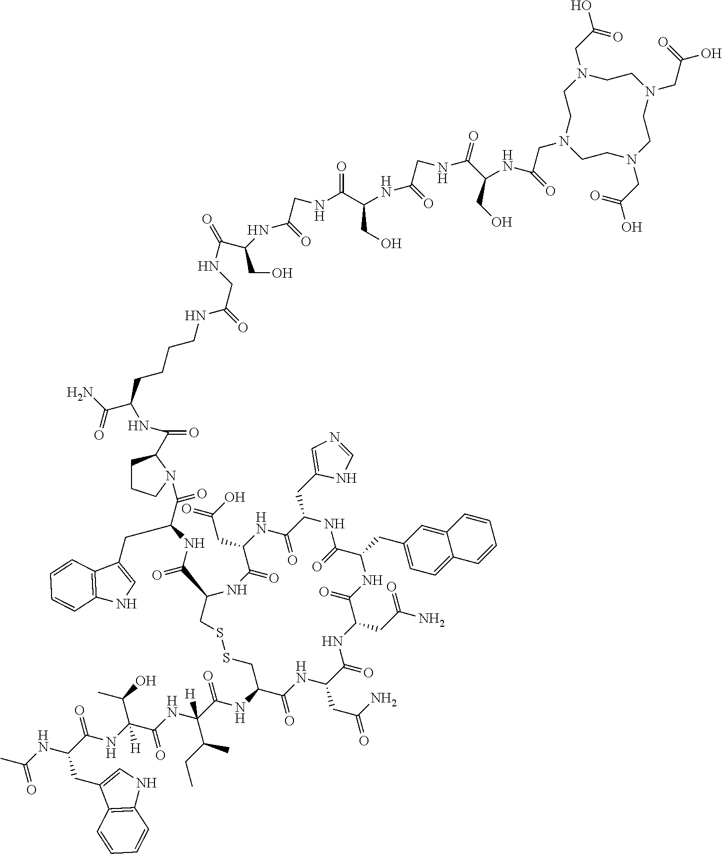 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CNC(=O)[C@H](CO)NC(=O)CNC(=O)[C@H](CO)NC(=O)CNC(=O)[C@H](CO)NC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

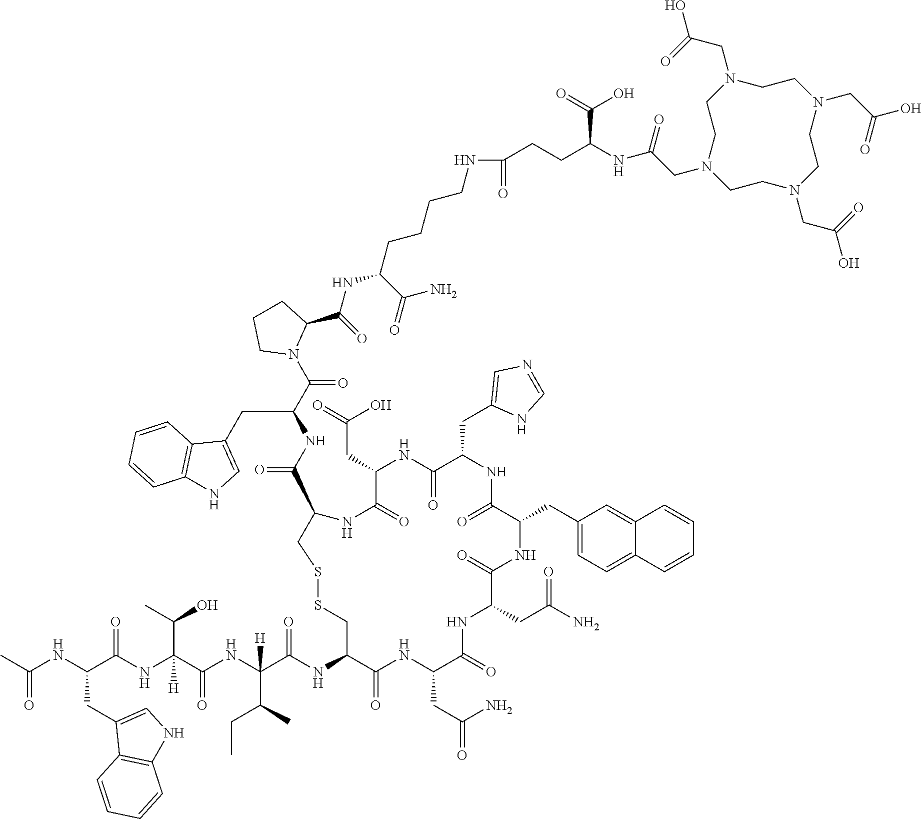 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CC[C@H](NC(=O)CN2CCN(CC(=O)O)CCN(CC(=O)O)CCN(CC(=O)O)CC2)C(=O)O)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

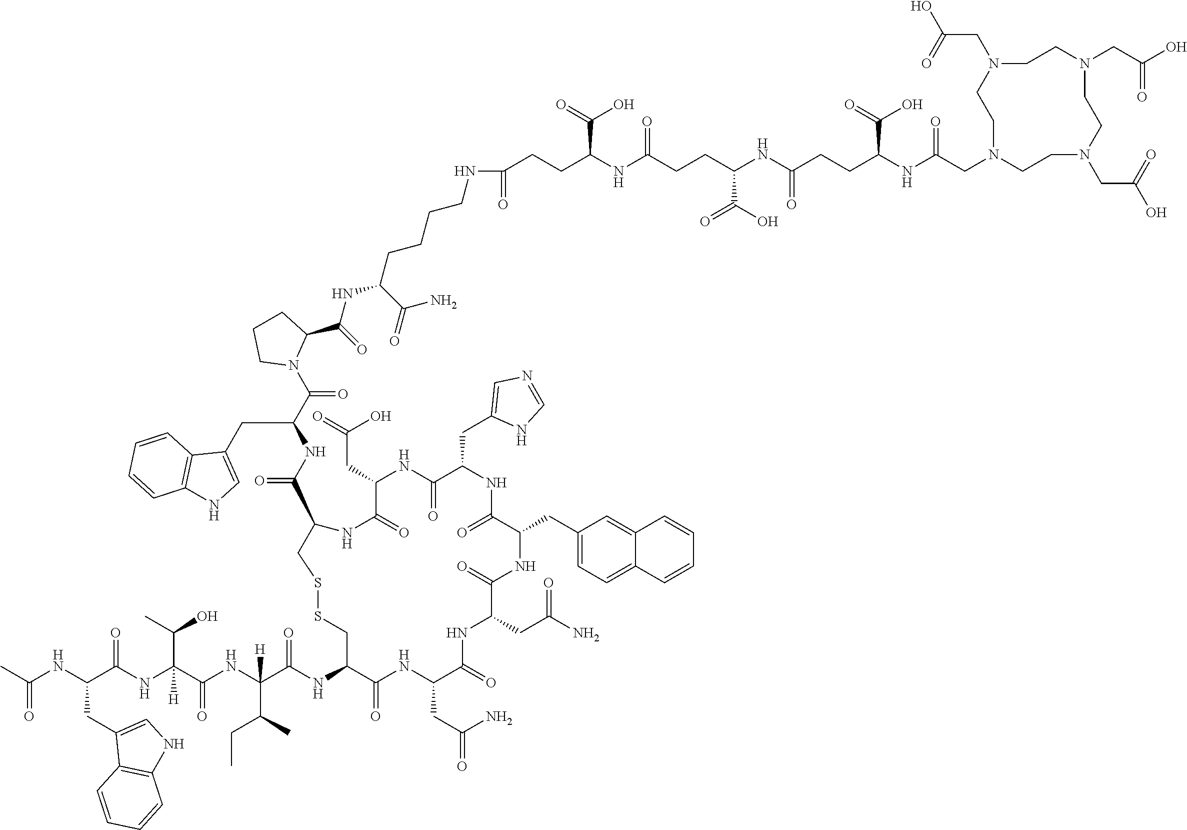 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

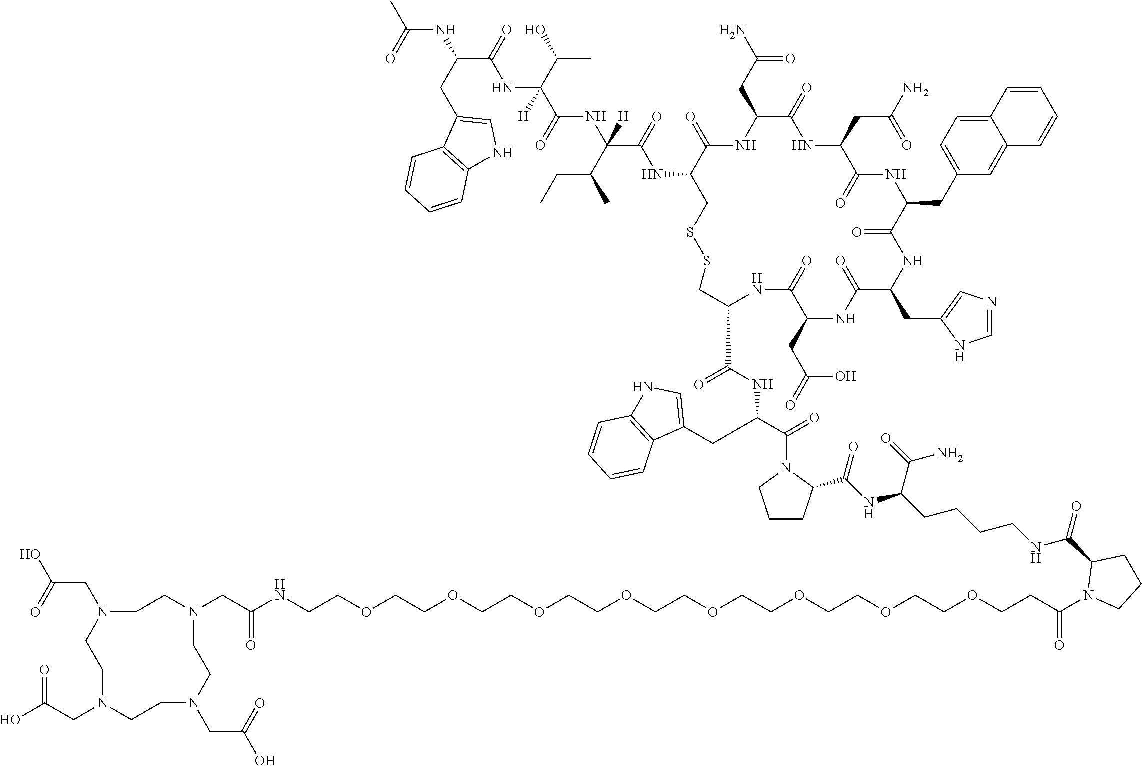 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)[C@H]2CCCN2C(=O)CCOCCOCCOCCOCCOCCOCCOCCOCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

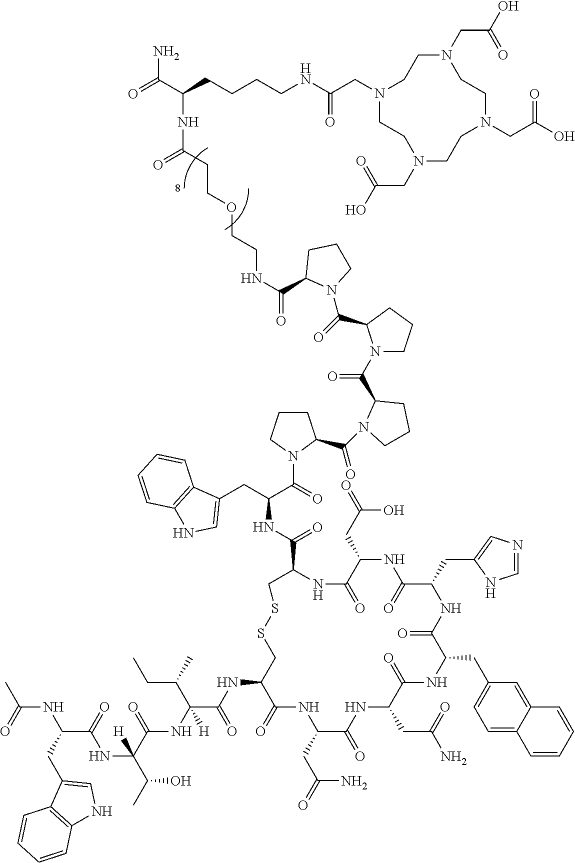

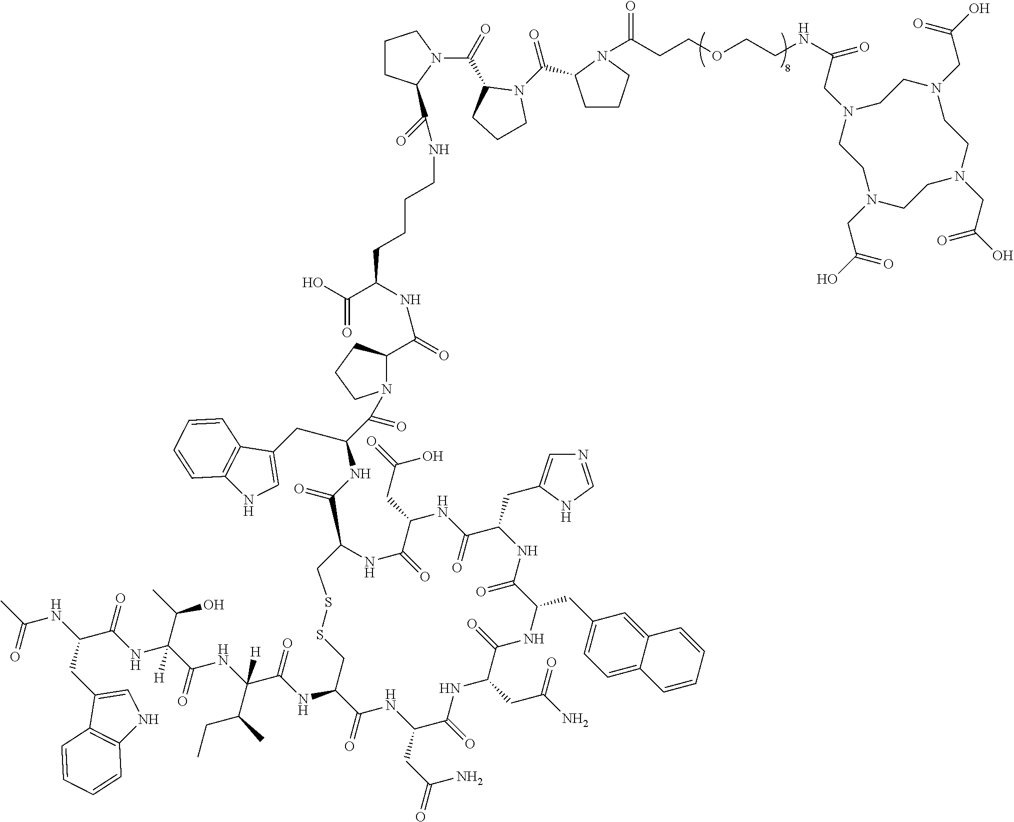

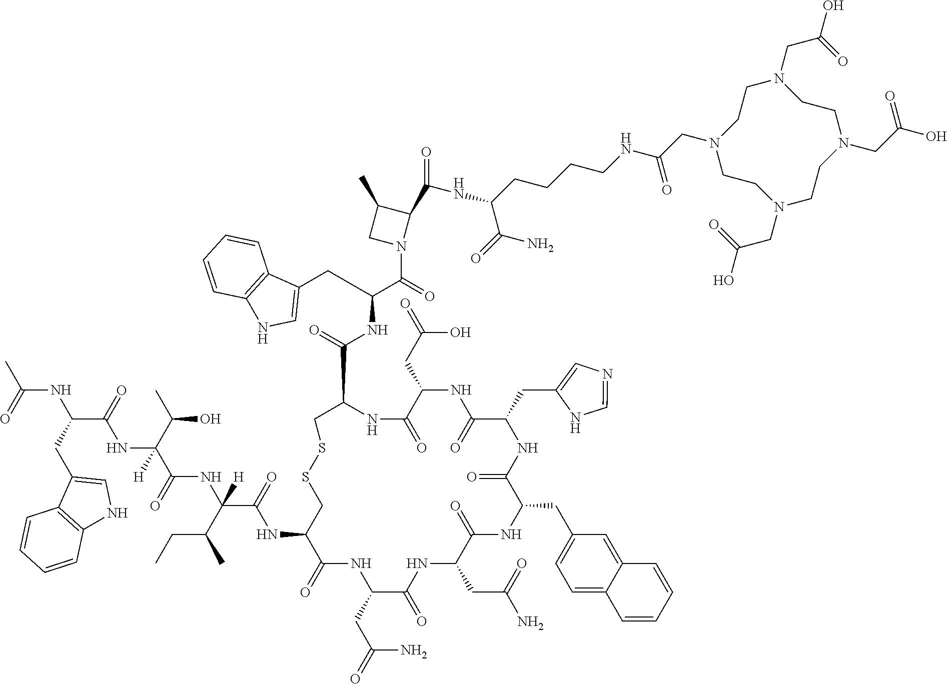 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@@H](C)[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

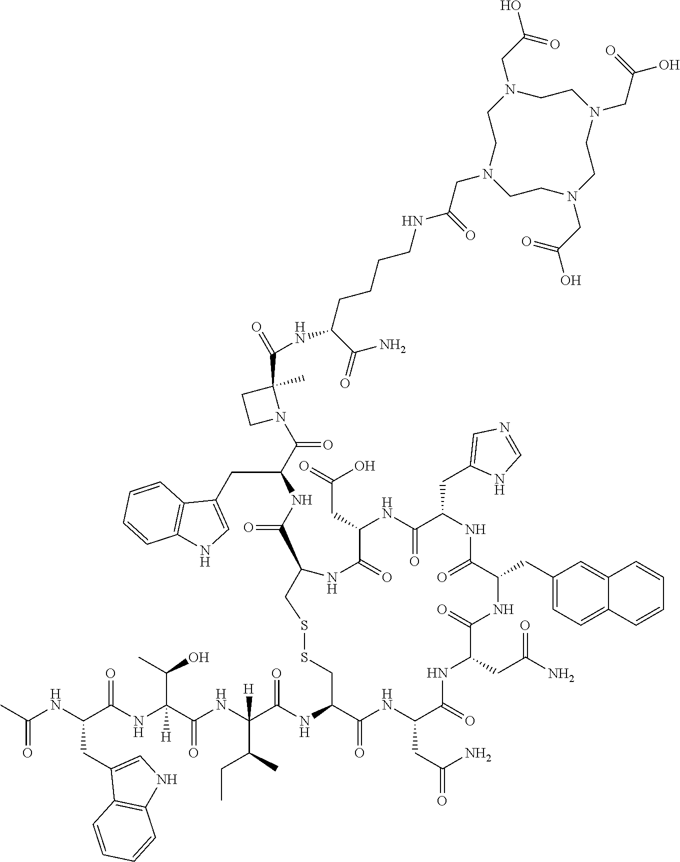 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CC[C@@]2(C)C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

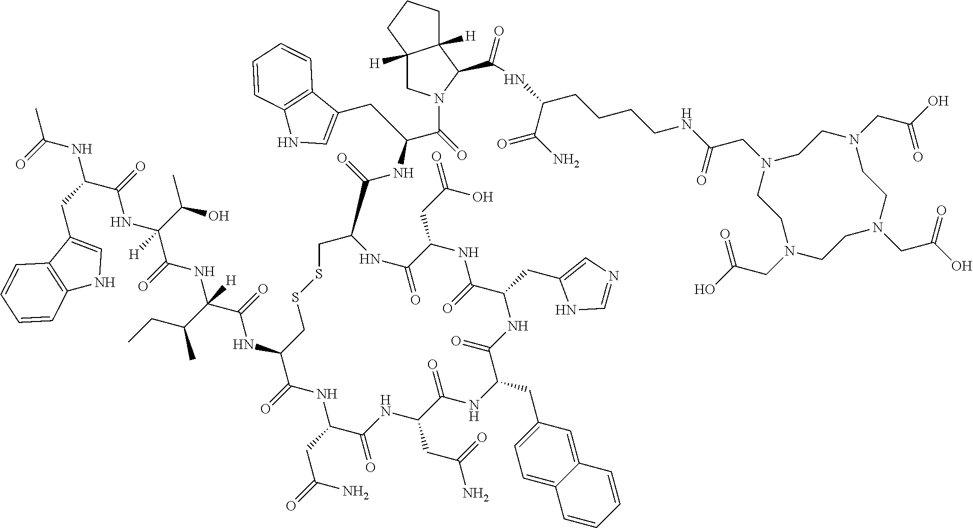 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2C[C@@H]3CCC[C@@H]3[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

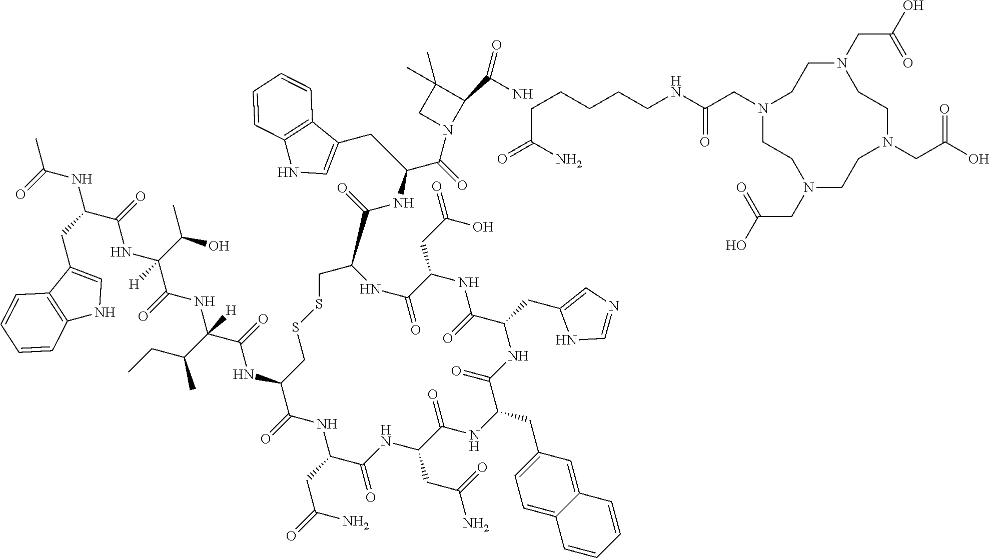

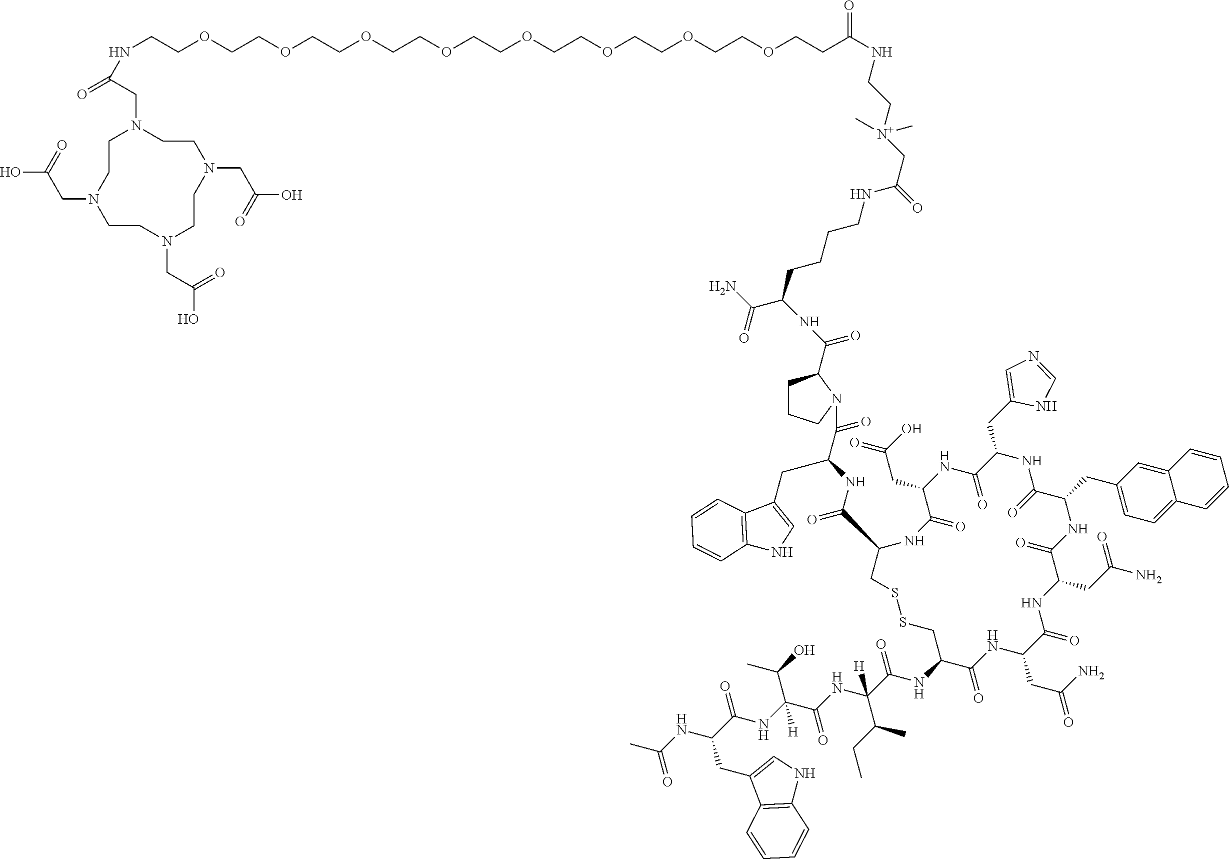 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)C[N+](C)(C)CCNC(=O)CCOCCOCCOCCOCCOCCOCCOCCOCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

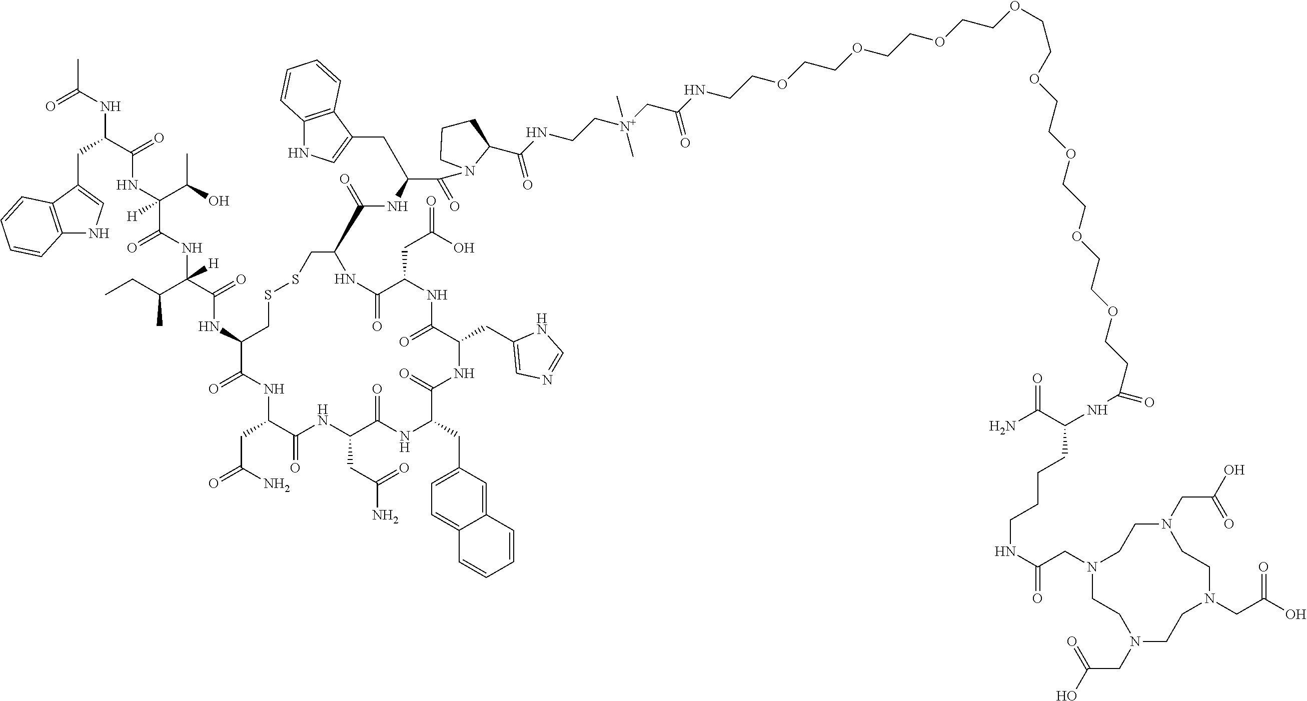 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NCC[N+](C)(C)CC(=O)NCCOCCOCCOCCOCCOCCOCCOCCOCCC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

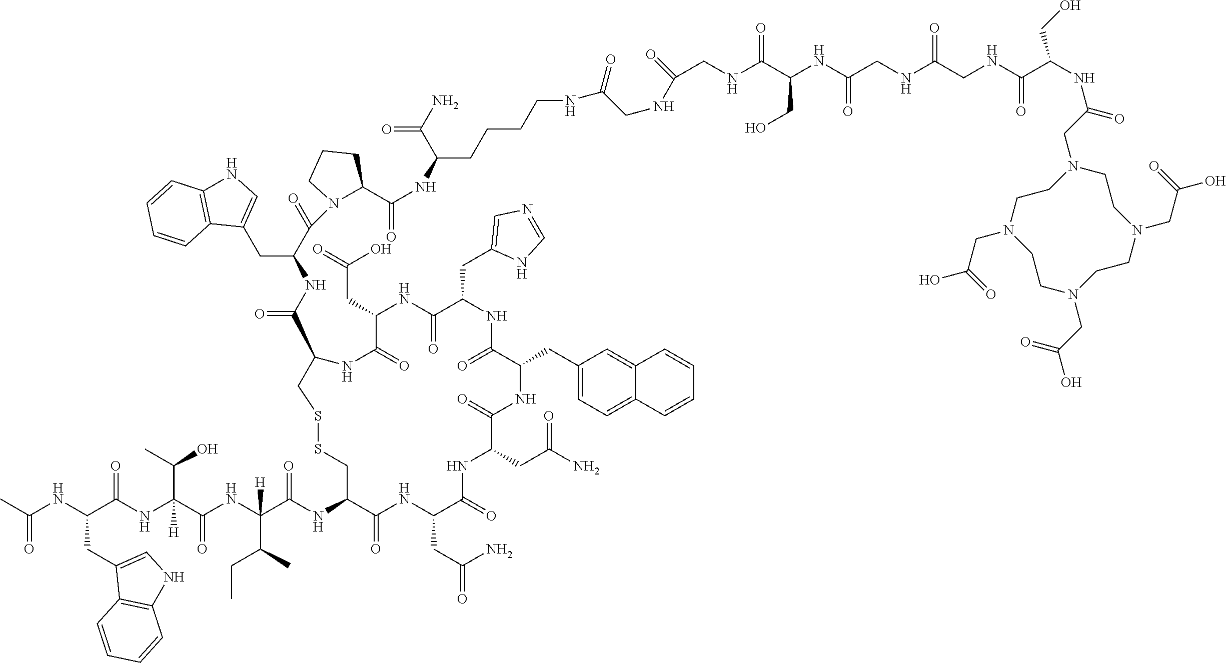 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CNC(=O)CNC(=O)[C@H](CO)NC(=O)CNC(=O)CNC(=O)[C@H](CO)NC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

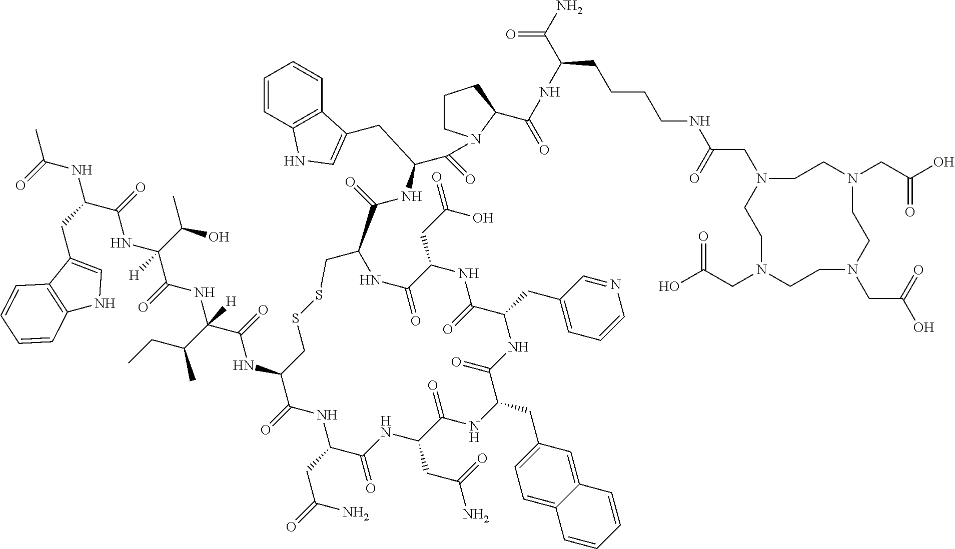 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cccnc2)NC(=O)[C@H](Cc2ccc3ccccc3c2)NC(=O)[C@H](CC(N)=O)NC(=O)[C@H](CC(N)=O)NC1=O]

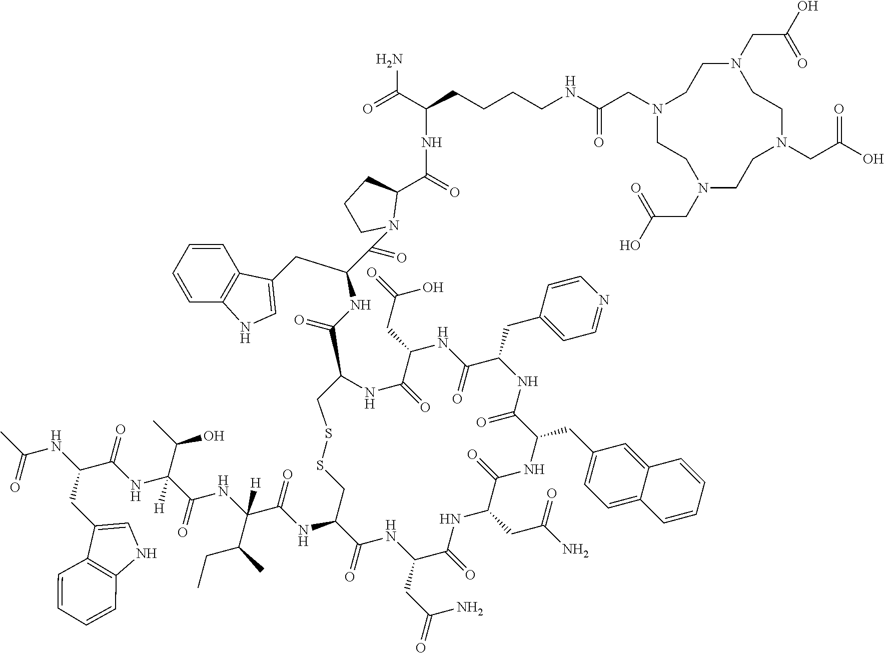 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2ccncc2)NC(=O)[C@H](Cc2ccc3ccccc3c2)NC(=O)[C@H](CC(N)=O)NC(=O)[C@H](CC(N)=O)NC1=O]

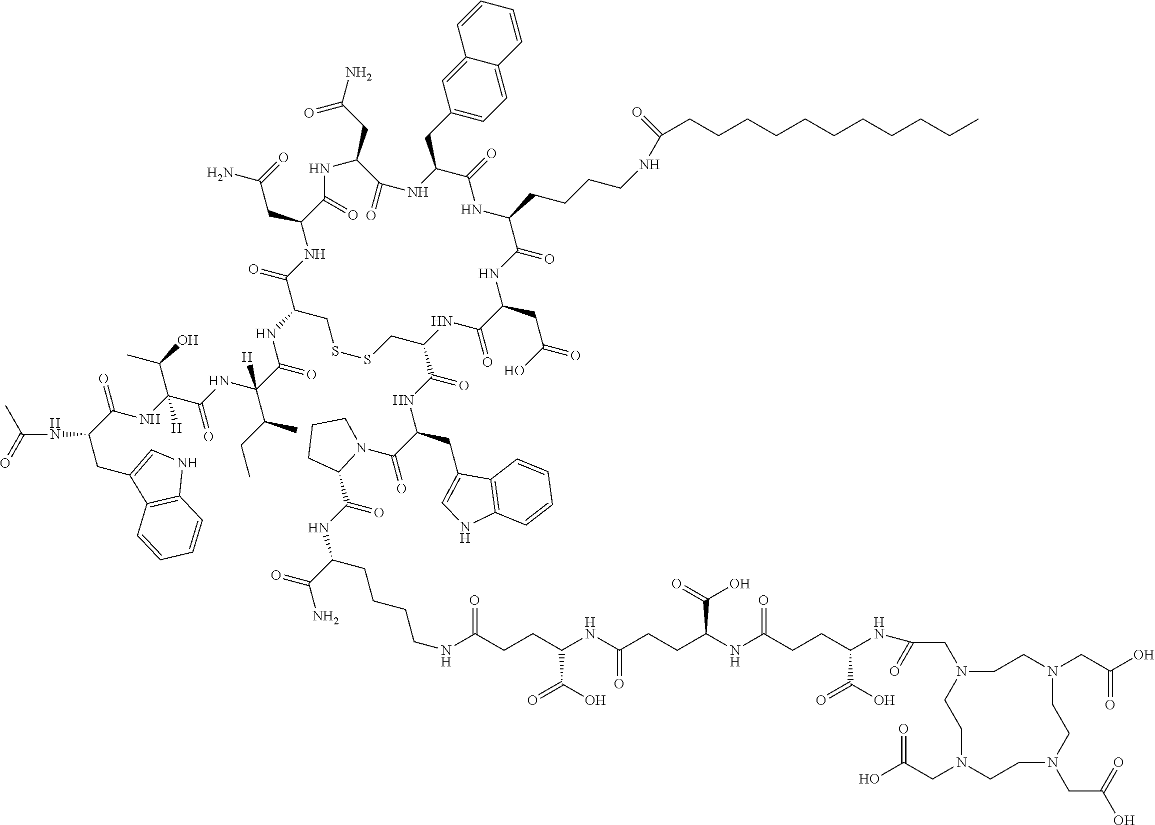

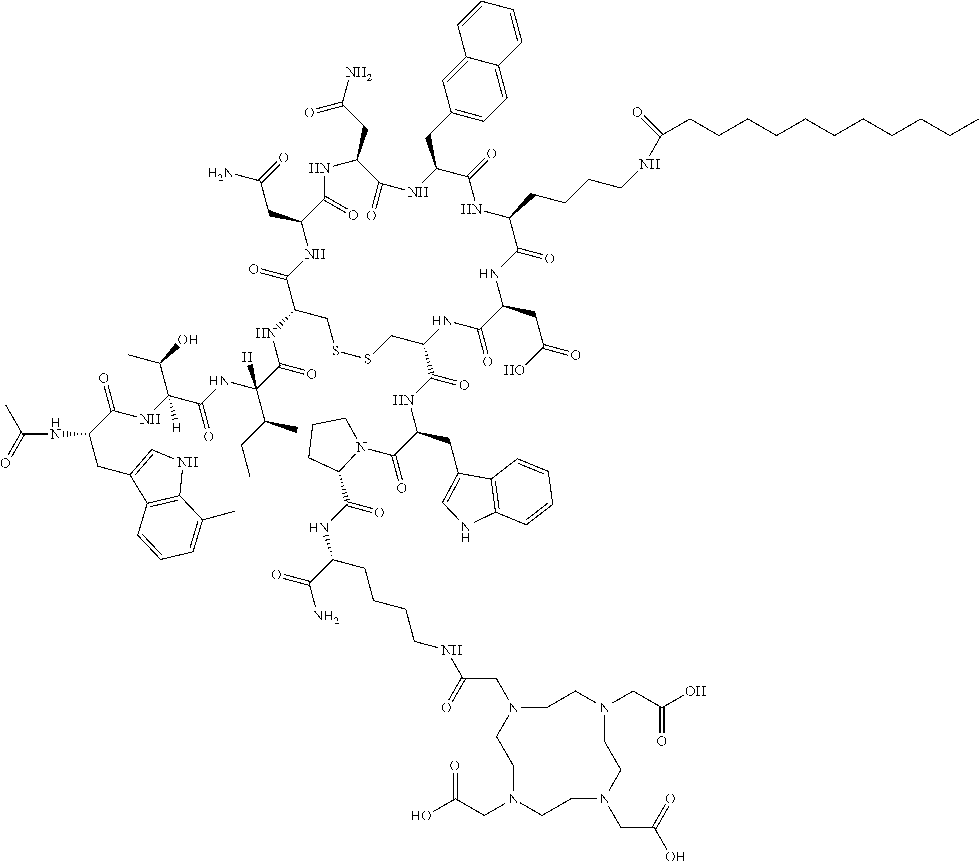 [SMILES: CCCCCCCCCCCC(=O)NCCCC[C@@H]1NC(=O)[C@H](Cc2ccc3ccccc3c2)NC(=O)[C@H](CC(N)=O)NC(=O)[C@H](CC(N)=O)NC(=O)[C@@H](NC(=O)[C@@H](NC(=O)[C@@H](NC(=O)[C@H](Cc2c[nH]c3c(C)cccc23)NC(C)=O)[C@@H](C)O)[C@@H](C)CC)CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC1=O]

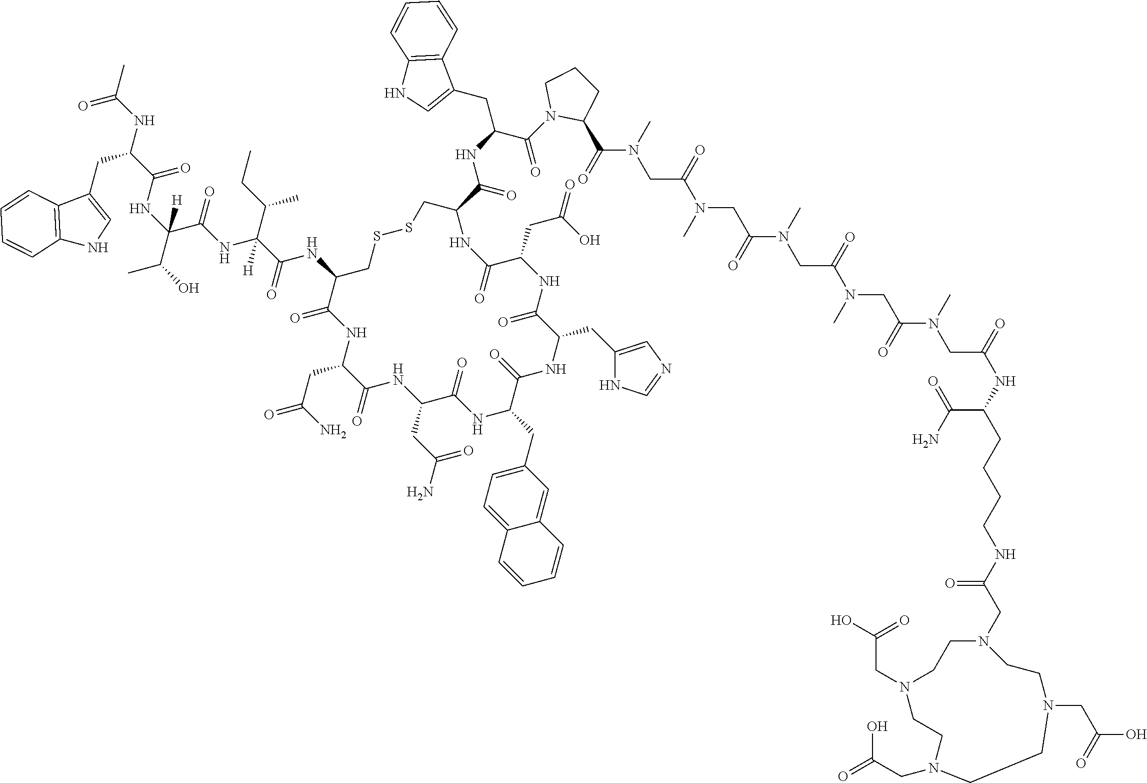 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N(C)CC(=O)N(C)CC(=O)N(C)CC(=O)N(C)CC(=O)N(C)CC(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

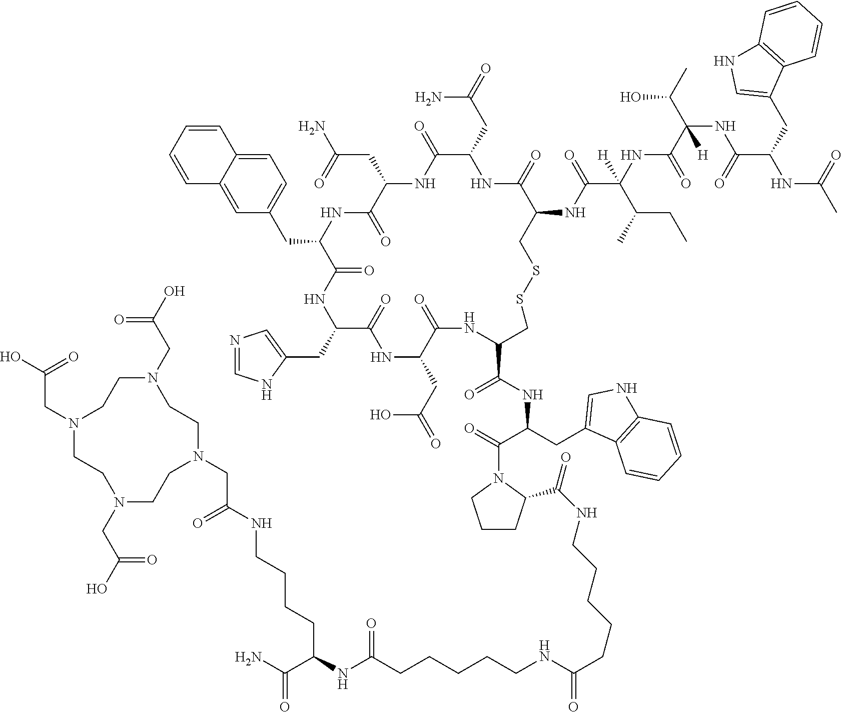

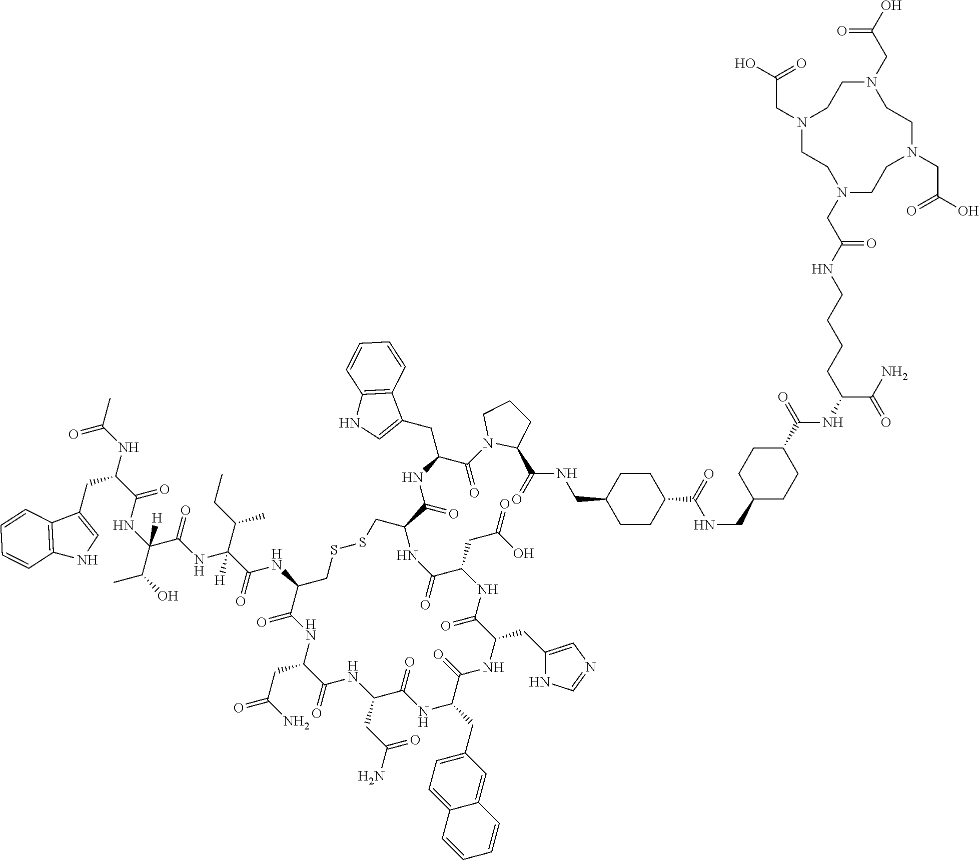 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)NC[C@H]2CC[C@H](C(=O)NC[C@H]3CC[C@H](C(=O)N[C@H](CCCCNC(=O)CN4CCN(CC(=O)O)CCN(CC(=O)O)CCN(CC(=O)O)CC4)C(N)=O)CC3)CC2)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CC(N)=O)NC1=O]

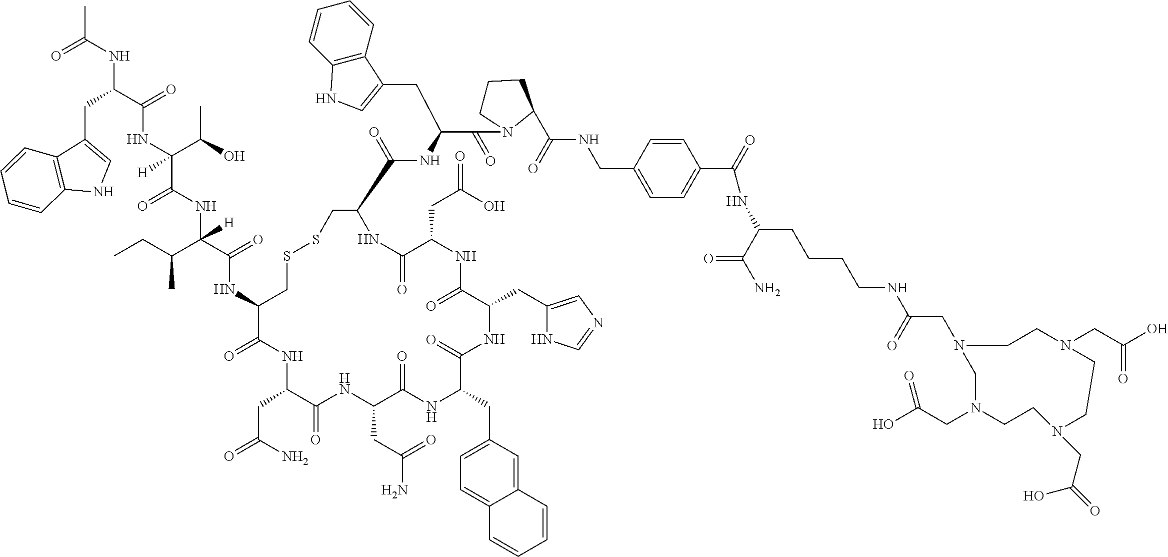

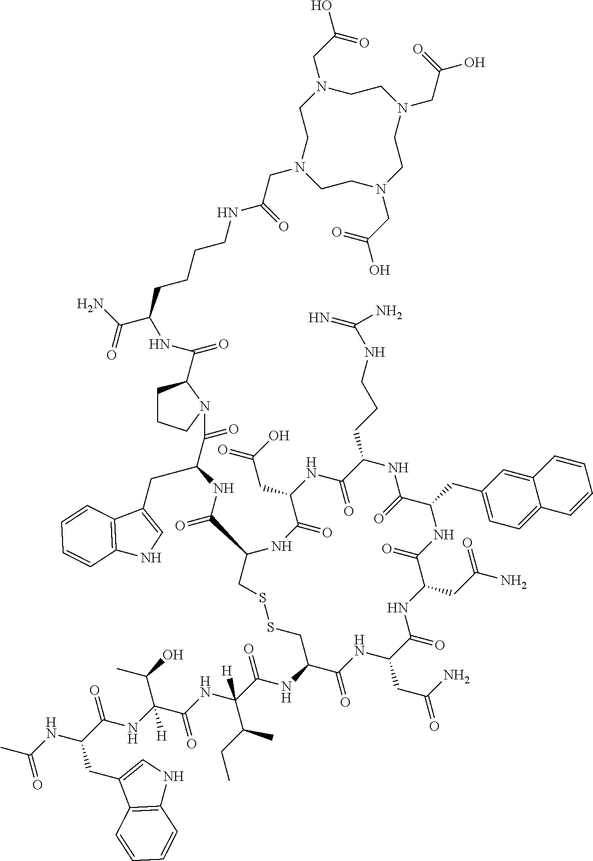 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CCCNC(=N)N)NC(=O)[C@H](Cc2ccc3ccccc3c2)NC(=O)[C@H](CC(N)=O)NC(=O)[C@H](CC(N)=O)NC1=O]

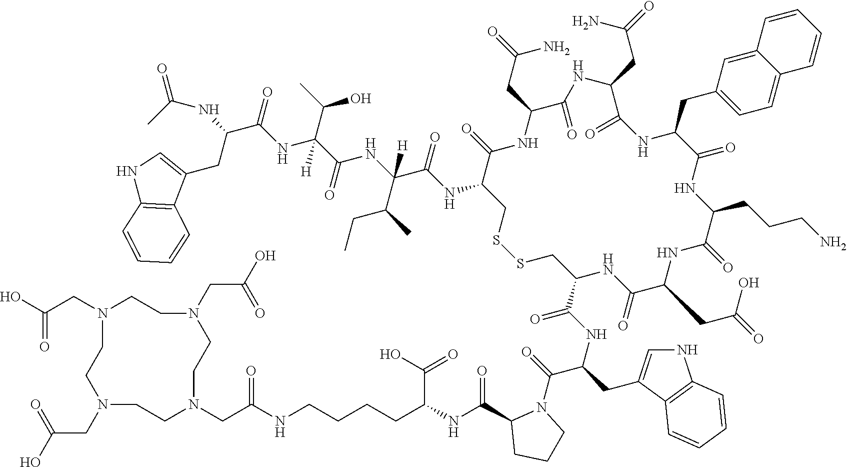 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(=O)O)NC(=O)[C@H](CC(=O)O)NC(=O)[C@H](CCCN)NC(=O)[C@H](Cc2ccc3ccccc3c2)NC(=O)[C@H](CC(N)=O)NC(=O)[C@H](CC(N)=O)NC1=O]

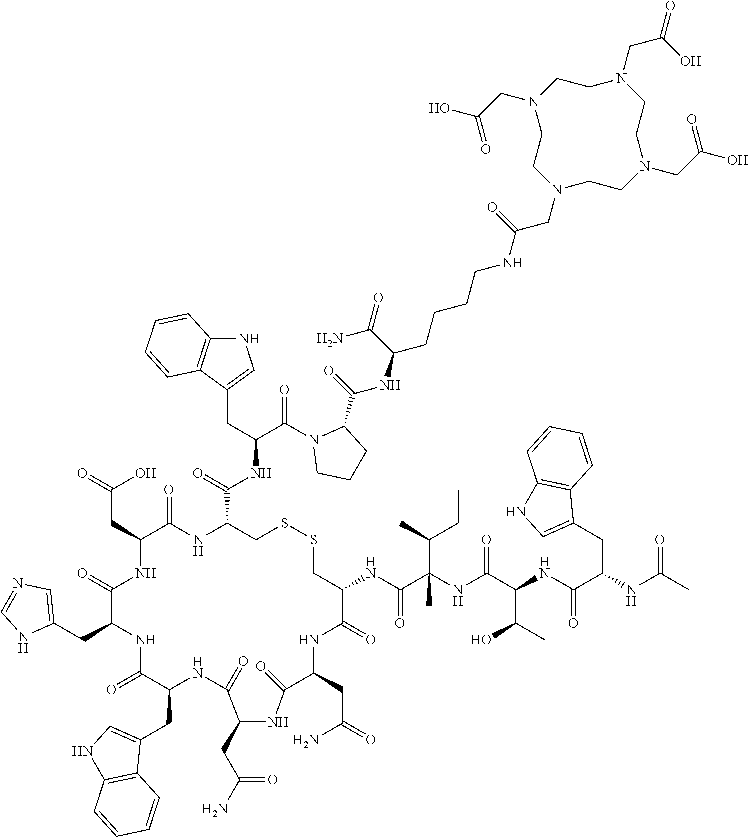 [SMILES: CC[C@H](C)[C@](C)(NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

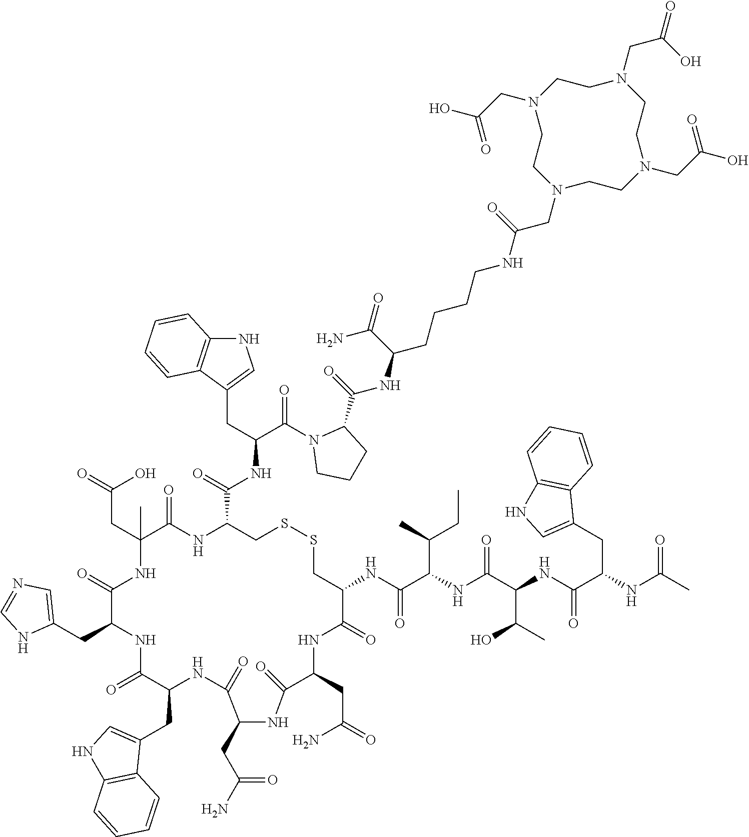 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)C(C)(CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CC(N)=O)NC1=O]

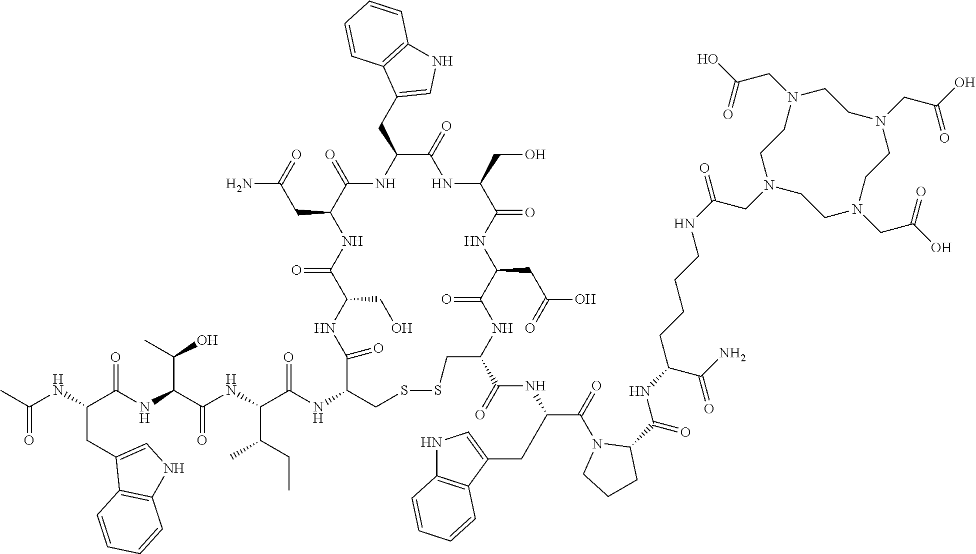 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](Cc2c[nH]c3ccccc23)NC(=O)[C@H](CC(N)=O)NC(=O)[C@H](CO)NC1=O]

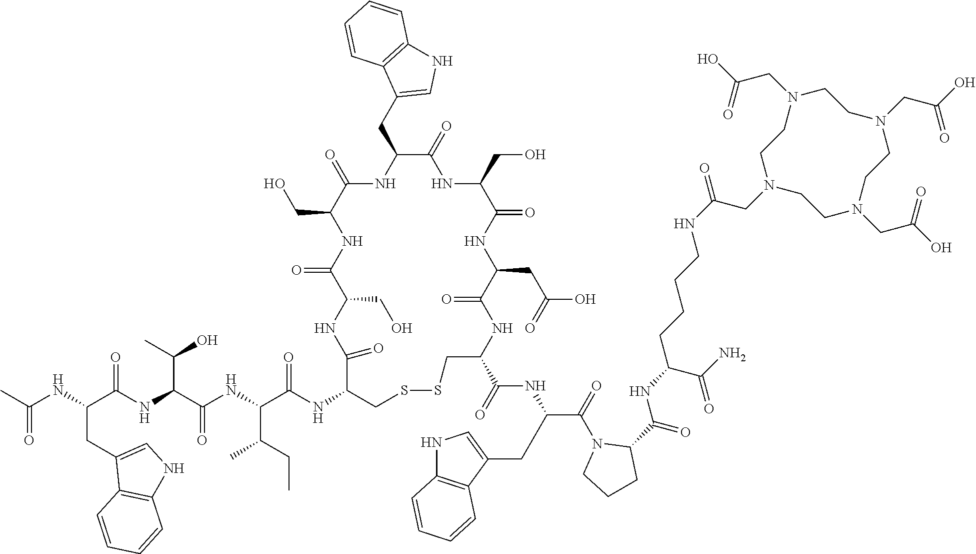 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](Cc2c[nH]c3ccccc23)NC(=O)[C@H](CO)NC(=O)[C@H](CO)NC1=O]

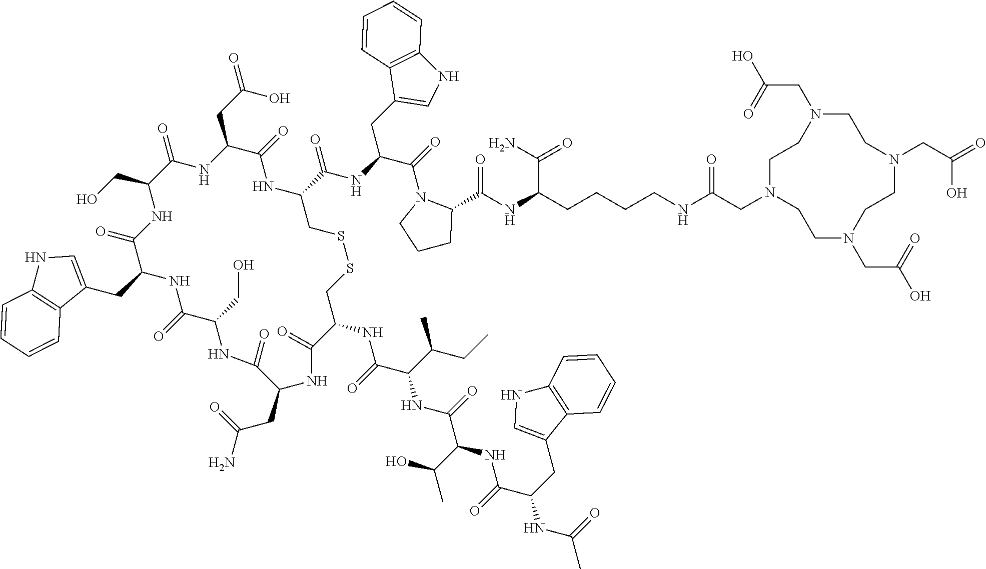 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](CO)NC(=O)[C@H](Cc2c[nH]c3ccccc23)NC(=O)[C@H](CO)NC(=O)[C@H](CC(N)=O)NC1=O]

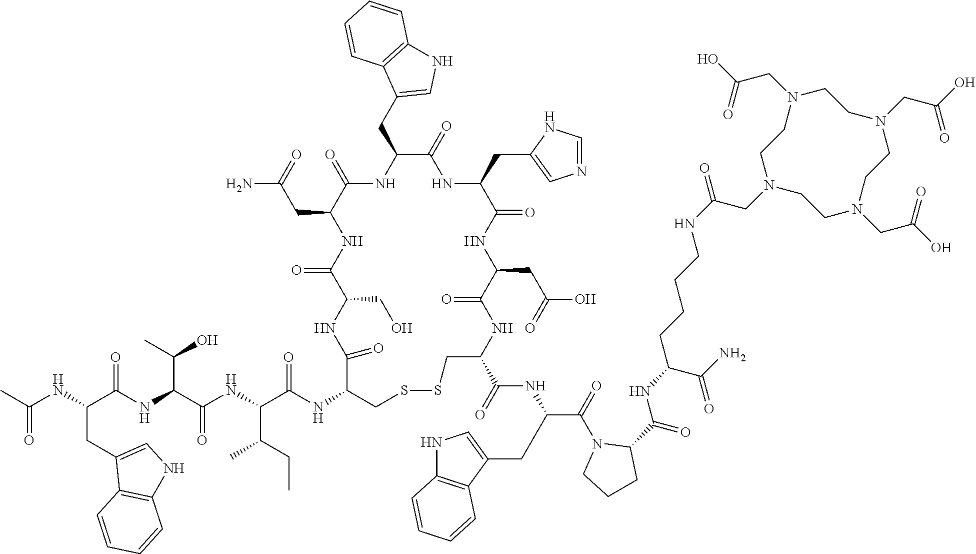 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CC(N)=O)NC(=O)[C@H](CO)NC1=O]

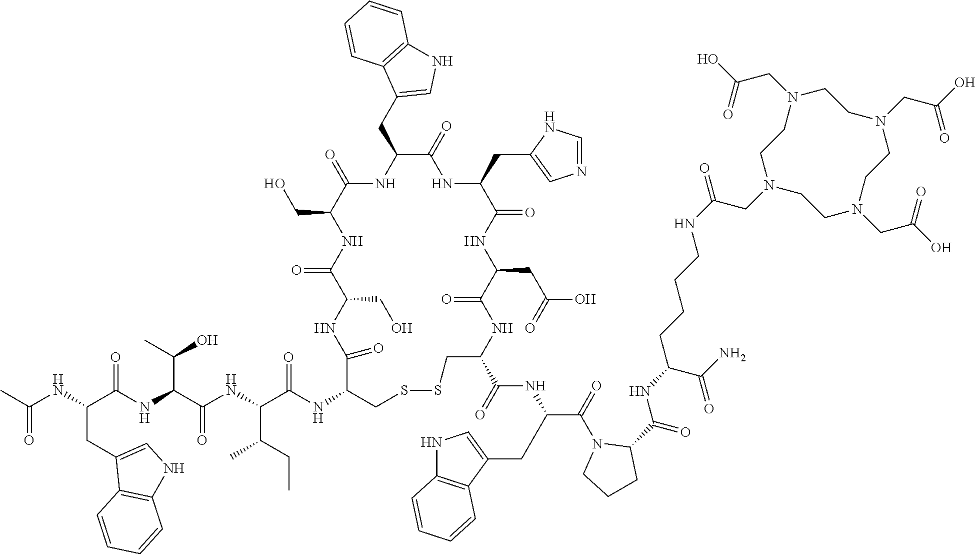 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2c[nH]c3ccccc23)NC(=O)[C@H](CO)NC(=O)[C@H](CO)NC1=O]

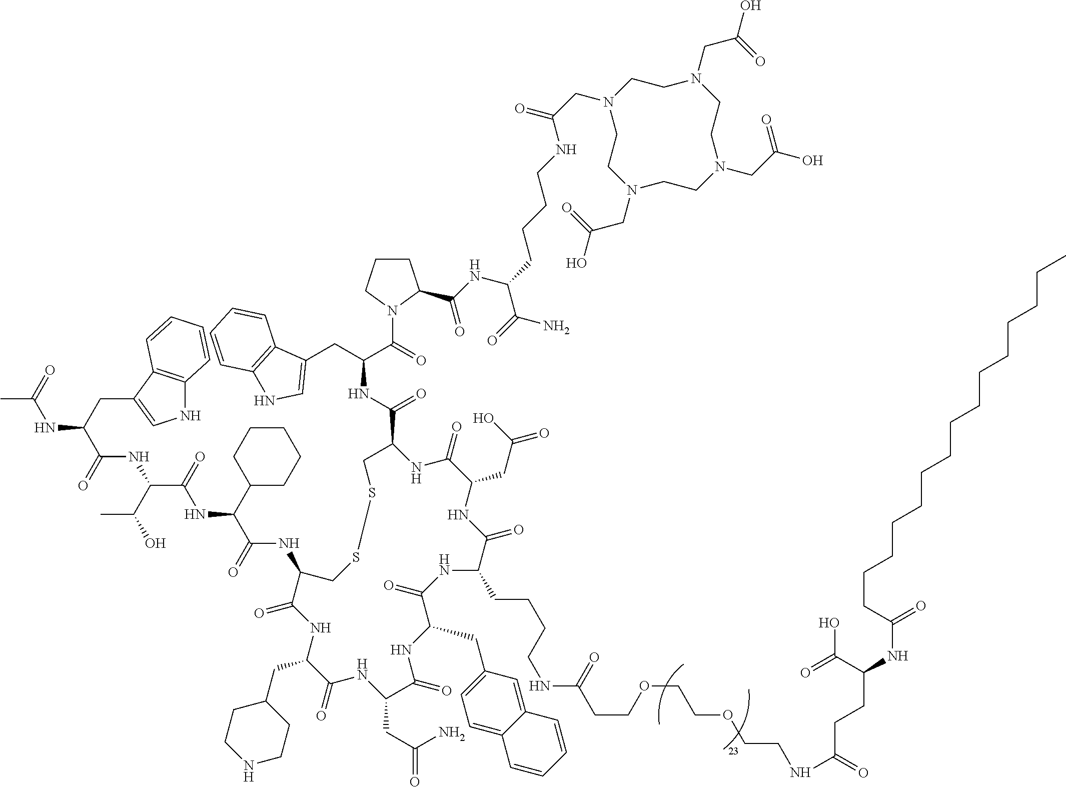

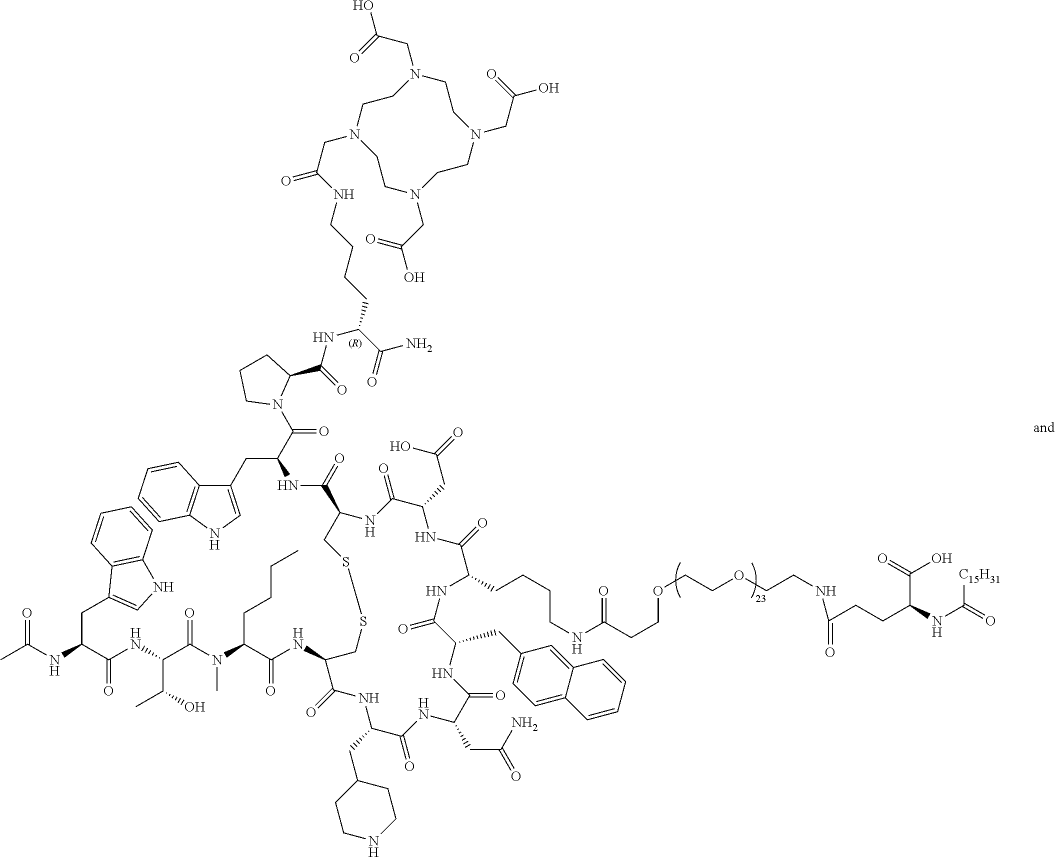

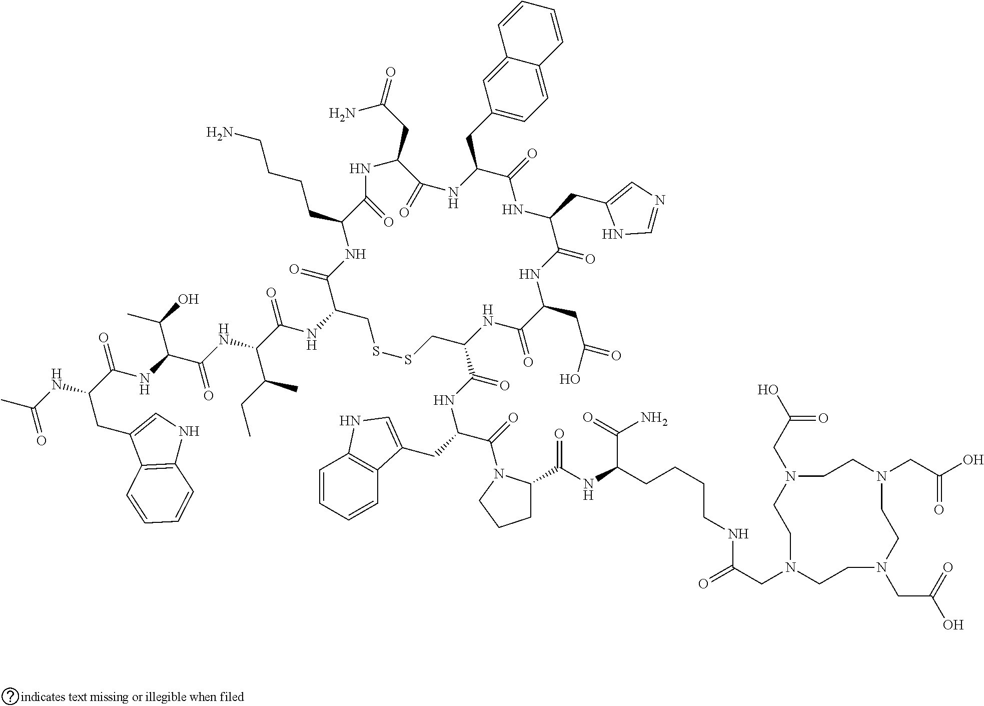 [SMILES: CC[C@H](C)[C@H](NC(=O)[C@@H](NC(=O)[C@H](Cc1c[nH]c2ccccc12)NC(C)=O)[C@@H](C)O)C(=O)N[C@H]1CSSC[C@@H](C(=O)N[C@@H](Cc2c[nH]c3ccccc23)C(=O)N2CCC[C@H]2C(=O)N[C@H](CCCCNC(=O)CN2CCN(CC(=O)O)CCN(CC(=O)O)CCN(CC(=O)O)CC2)C(N)=O)NC(=O)[C@H](CC(=O)O)NC(=O)[C@H](Cc2cnc[nH]2)NC(=O)[C@H](Cc2ccc3ccccc3c2)NC(=O)[C@H](CC(N)=O)NC(=O)[C@H](CCCCN)NC1=O]

19. (canceled)

20. The cyclic peptide of claim 1, or a pharmaceutically acceptable salt thereof, wherein the cyclic peptide further comprises a radionuclide.

21. The cyclic peptide of claim 20, or a pharmaceutically acceptable salt thereof, wherein the radionuclide is selected from C-11, N-13, 0-15, F-18, P-32, Sc-47, Co-57, Cu-60, Cu-67, Cu-64, Ga-66, Ga-67, Ga-68, Br-76, Br-77, Kr-81m, Rb-82, Y-86, Zr-89, Sr-89, Y-86, Y-90, Sr-92, Tc-99m, Pd-103, Ac-227, Rh-105, Ag-111, In-111, 1-124, 1-131, Pr-142, Pm-149, Sm-153, Gd-159, Ho-166, Lu-177, Re-186, Re-188, Ir-194, Pt-199, Tl-201, Pb-203, At-211, Pb-212, Bi-212, Bi-213, Ra-223, Ac-225, Th-227, Lu-175, and In-115.

22. (canceled)

23. A pharmaceutical composition comprising the cyclic peptide of claim 1, or a pharmaceutically acceptable salt thereof, and a pharmaceutically acceptable carrier.

24. A method of treating cancer in a subject in need thereof comprising administering to the subject the cyclic peptide of claim 1, or a pharmaceutically acceptable salt thereof.

25. The method of claim 24, wherein the cancer is a DLL3-mediated cancer.

26. The method of claim 24, wherein the cancer is a neuroendocrine neoplasm, melanoma, or primary brain cancer.

27. The method of claim 26, wherein the neuroendocrine neoplasm is selected from small cell lung cancer (SCLC), medullary thyroid carcinoma (MTC), large cell neuroendocrine cancer (LCNEC), gastroenteropancreatic neuroendocrine carcinoma (GEP NEC), neuroendocrine prostate cancer (NEPC), small cell prostate cancer (SCPC), Merkel cell carcinoma (MCC), neuroendocrine cervical carcinoma, and Grade 3 neuroendocrine tumors (NETs).

28. A peptide having binding specificity for DLL3, wherein the peptide binds to one or more amino acids of A81, L83, G106, A85, and R61 of a DLL3 amino acid sequence of SEQ ID NO: 1, wherein the peptide comprises Formula A of the amino acid sequence: 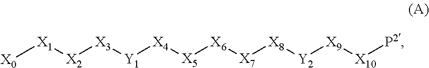
or a pharmaceutically acceptable salt thereof,
wherein:
X₀ is any natural or unnatural amino acid or X₀ is absent;
X₁ is selected from Trp, 7Aza-Trp, 1Me-Trp, 5OH-Trp, 5OMe-Trp, 7OMe-Trp, 7Me-Trp, 5F-Trp, 7Cl-Trp, alpha-Me-Trp, and NMe-Trp;
X₂ and X₃ are each independently any natural or unnatural amino acids;
Y₁ is Cys;
X₄, X₅, X₆, X₇ and X₈ are each independently any natural or unnatural amino acids;
Y₂ is Cys;
X₉ is selected from Trp, 7Aza-Trp, 1Me-Trp, 5OH-Trp, 5OMe-Trp, 7OMe-Trp, 7Me-Trp, 5F-Trp, 7Cl-Trp, alpha-Me-Trp, and NMe-Trp;
X₁₀ is selected from Pro, alpha-Me-Pro, trans4Fluoro-Pro, cis4Fluoro-Pro, trans40H-Pro, cis4OH-Pro, 5,5-diMe-Pro, trans4NH2-Pro, and cis4NH2-Pro;
P² is selected from -L²-Chelator or 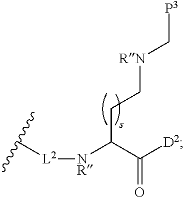
D² is OH or NH₂;
L² is absent or selected from: 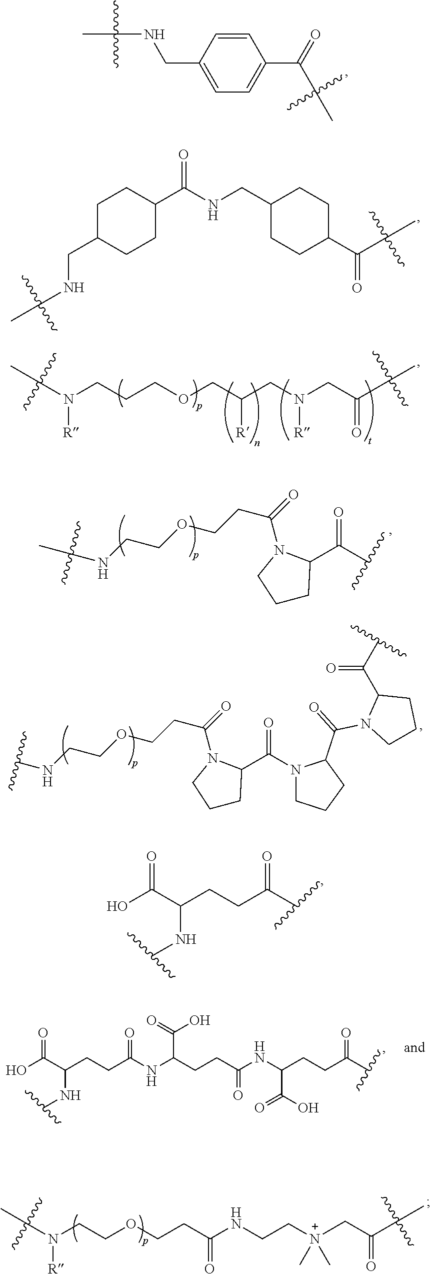
wherein the amino group of L² connects to the carbonyl group of P² or Chelator to form an amide bond;
P³ is selected from -L³-Chelator, 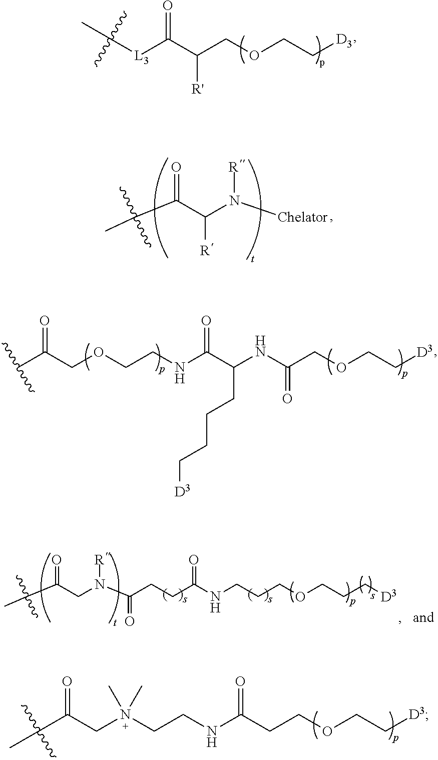
L³ is absent or independently selected from 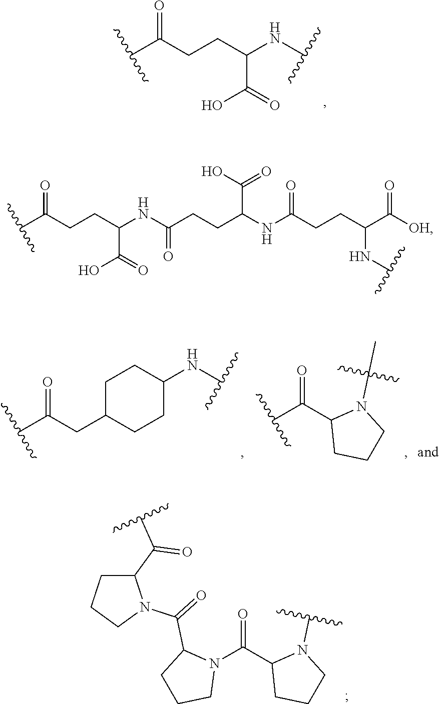 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]
wherein the carbonyl group of L³ connects to an amine group of P² to form an amide bond;
D³ is independently —NR″-Chelator or 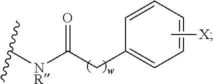
each R′ is independently selected from H, C(O)OH, (CH₂)OH and NHAc;
each R″ is independently selected from H and CH₃;
X is H or halogen;
each n is independently an integer from 0 to 16;
each p is independently an integer from 0 to 24;
each s is independently an integer from 0 to 16;
each t is independently 1, 2, 3, 4, 5, or 6; and
w is selected from 1, 2, or 3;
wherein the cyclic peptide is cyclized via a linker between Y₁ and Y₂; and
wherein the cyclic peptide binds to DLL3.

29-42. (canceled)


SEQUENCE LISTING (316 sequences)

SEQ ID NO: 1 - protein, 618 aa, synthetic construct
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 2 - protein, 466 aa, synthetic construct
AGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYL

SEQ ID NO: 3 - protein, 13 aa, synthetic construct
MWTICADWAQCWP

SEQ ID NO: 4 - protein, 12 aa, synthetic construct
WTICADWAQCWP

SEQ ID NO: 5 - protein, 13 aa, synthetic construct
MWTICNNWHDCWP

SEQ ID NO: 6 - protein, 13 aa, synthetic construct
MWKICSDWQNCWP

SEQ ID NO: 7 - protein, 13 aa, synthetic construct
MWHICQEWATCWP

SEQ ID NO: 8 - protein, 13 aa, synthetic construct
MWTLCDTWDTCFP

SEQ ID NO: 9 - protein, 13 aa, synthetic construct
MWTLCDNWQTCWP

SEQ ID NO: 10 - protein, 13 aa, synthetic construct
MWQLCEWNNCWPV

SEQ ID NO: 11 - protein, 13 aa, synthetic construct
MWVLCEWHECWPG

SEQ ID NO: 12 - protein, 13 aa, synthetic construct
MDCNFFDCDAWTW

SEQ ID NO: 13 - protein, 13 aa, synthetic construct
MWMLCDWDKDCWP

SEQ ID NO: 14 - protein, 13 aa, synthetic construct
MVWVPCEWDDCFY

SEQ ID NO: 15 - protein, 13 aa, synthetic construct
MSDCWLQWNCPFD

SEQ ID NO: 16 - protein, 12 aa, synthetic construct
WTICNNWHDCWP

SEQ ID NO: 17 - protein, 13 aa, synthetic construct
AWTICNNWHDCWP

SEQ ID NO: 18 - protein, 13 aa, synthetic construct
MWTICNNWHACWP

SEQ ID NO: 19 - protein, 13 aa, synthetic construct
MWTICNNWHDCAP

SEQ ID NO: 20 - protein, 13 aa, synthetic construct
MWTICNNWHDCWA

SEQ ID NO: 21 - protein, 13 aa, synthetic construct
MWAICNNWHDCWP

SEQ ID NO: 22 - protein, 13 aa, synthetic construct
MWTACNNWHDCWP

SEQ ID NO: 23 - protein, 13 aa, synthetic construct
MWTICANWHDCWP

SEQ ID NO: 24 - protein, 13 aa, synthetic construct
MWTICADWAQCWP

SEQ ID NO: 25 - protein, 12 aa, synthetic construct
WTICADWAQCWP

SEQ ID NO: 26 - protein, 13 aa, synthetic construct
MWTICNNWHDCWP

SEQ ID NO: 27 - protein, 13 aa, synthetic construct
MWKICSDWQNCWP

SEQ ID NO: 28 - protein, 13 aa, synthetic construct
MWHICQEWATCWP

SEQ ID NO: 29 - protein, 13 aa, synthetic construct
MWTLCDTWDTCFP

SEQ ID NO: 30 - protein, 13 aa, synthetic construct
MWTLCDNWQTCWP

SEQ ID NO: 31 - protein, 13 aa, synthetic construct
MWQLCEWNNCWPV

SEQ ID NO: 32 - protein, 13 aa, synthetic construct
MWVLCEWHECWPG

SEQ ID NO: 33 - protein, 13 aa, synthetic construct
MDCNFFDCDAWTW

SEQ ID NO: 34 - protein, 13 aa, synthetic construct
MWMLCDWDKDCWP

SEQ ID NO: 35 - protein, 13 aa, synthetic construct
MVWVPCEWDDCFY

SEQ ID NO: 36 - protein, 13 aa, synthetic construct
MSDCWLQWNCPFD

SEQ ID NO: 37 - protein, 12 aa, synthetic construct
WTICNNWHDCWP

SEQ ID NO: 38 - protein, 13 aa, synthetic construct
AWTICNNWHDCWP

SEQ ID NO: 39 - protein, 13 aa, synthetic construct
MWTICANWHDCWP

SEQ ID NO: 40 - protein, 13 aa, synthetic construct
MWTICNNWHACWP

SEQ ID NO: 41 - protein, 13 aa, synthetic construct
MWTICNNWHDCAP

SEQ ID NO: 42 - protein, 13 aa, synthetic construct
MWTICNNWHDCWA

SEQ ID NO: 43 - protein, 13 aa, synthetic construct
MWAICNNWHDCWP

SEQ ID NO: 44 - protein, 13 aa, synthetic construct
MWTACNNWHDCWP

SEQ ID NO: 45 - protein, 13 aa, synthetic construct
  REGION 5..11: note = Peptide is circular
  DISULFID 5..11: note = Disulfide bond
MWTICADWAQCWP

SEQ ID NO: 46 - protein, 12 aa, synthetic construct
  REGION 4..10: note = Peptide is circular
  DISULFID 4..10: note = Disulfide bond
WTICADWAQCWP

SEQ ID NO: 47 - protein, 13 aa, synthetic construct
  REGION 5..11: note = Peptide is circular
  DISULFID 5..11: note = Disulfide bond
MWTICNNWHDCWP

SEQ ID NO: 48 - protein, 13 aa, synthetic construct
  DISULFID 5..11: note = Disulfide bond
  REGION 5..11: note = Peptide is circular
MWKICSDWQNCWP

SEQ ID NO: 49 - protein, 13 aa, synthetic construct
  REGION 5..11: note = Peptide is circular
  DISULFID 5..11: note = Disulfide bond
MWHICQEWATCWP

SEQ ID NO: 50 - protein, 13 aa, synthetic construct
  DISULFID 5..11: note = Disulfide bond
  REGION 5..11: note = Peptide is circular
MWTLCDTWDTCFP

SEQ ID NO: 51 - protein, 13 aa, synthetic construct
  REGION 5..11: note = Peptide is circular
  DISULFID 5..11: note = Disulfide bond
MWTLCDNWQTCWP

SEQ ID NO: 52 - protein, 13 aa, synthetic construct
  DISULFID 5..10: note = Disulfide bond
  REGION 5..10: note = Peptide is circular
MWQLCEWNNCWPV

SEQ ID NO: 53 - protein, 13 aa, synthetic construct
  REGION 5..10: note = Peptide is circular
  DISULFID 5..10: note = Disulfide bond
MWVLCEWHECWPG

SEQ ID NO: 54 - protein, 13 aa, synthetic construct
  DISULFID 3..8: note = Disulfide bond
  REGION 3..8: note = Peptide is circular
MDCNFFDCDAWTW

SEQ ID NO: 55 - protein, 13 aa, synthetic construct
  REGION 5..11: note = Peptide is circular
  DISULFID 5..11: note = Disulfide bond
MWMLCDWDKDCWP

SEQ ID NO: 56 - protein, 13 aa, synthetic construct
  DISULFID 6..11: note = Disulfide bond
  REGION 6..11: note = Peptide is circular
MVWVPCEWDDCFY

SEQ ID NO: 57 - protein, 13 aa, synthetic construct
  REGION 4..10: note = Peptide is circular
  DISULFID 4..10: note = Disulfide bond
MSDCWLQWNCPFD

SEQ ID NO: 58 - protein, 12 aa, synthetic construct
  DISULFID 4..10: note = Disulfide bond
  REGION 4..10: note = Peptide is circular
WTICNNWHDCWP

SEQ ID NO: 59 - protein, 13 aa, synthetic construct
  REGION 5..11: note = Peptide is circular
  DISULFID 5..11: note = Disulfide bond
AWTICNNWHDCWP

SEQ ID NO: 60 - protein, 13 aa, synthetic construct
  REGION 5..11: note = Peptide is circular
  DISULFID 5..11: note = Disulfide bond
MWTICANWHDCWP

SEQ ID NO: 61 - protein, 13 aa, synthetic construct
  DISULFID 5..11: note = Disulfide bond
  REGION 5..11: note = Peptide is circular
MWTICNNWHACWP

SEQ ID NO: 62 - protein, 13 aa, synthetic construct
  REGION 5..11: note = Peptide is circular
  DISULFID 5..11: note = Disulfide bond
MWTICNNWHDCAP

SEQ ID NO: 63 - protein, 13 aa, synthetic construct
  REGION 5..11: note = Peptide is circular
  DISULFID 5..11: note = Disulfide bond
MWTICNNWHDCWA

SEQ ID NO: 64 - skipped

SEQ ID NO: 65 - protein, 13 aa, synthetic construct
  DISULFID 5..11: note = Disulfide bond
  REGION 5..11: note = Peptide is circular
MWAICNNWHDCWP

SEQ ID NO: 66 - protein, 13 aa, synthetic construct
  REGION 5..11: note = Peptide is circular
  DISULFID 5..11: note = Disulfide bond
MWTACNNWHDCWP

SEQ ID NO: 67 - protein, 13 aa, synthetic construct
  DISULFID 5..11: note = Disulfide bond
  REGION 5..11: note = Peptide is circular
MWTICADWAQCWP

SEQ ID NO: 68 - protein, 12 aa, synthetic construct
  REGION 4..10: note = Peptide is circular
  DISULFID 4..10: note = Disulfide bond
WTICADWAQCWP

SEQ ID NO: 69 - protein, 13 aa, synthetic construct
  DISULFID 5..11: note = Disulfide bond
  REGION 5..11: note = Peptide is circular
MWTICNNWHDCWP

SEQ ID NO: 70 - protein, 13 aa, synthetic construct
  REGION 5..11: note = Peptide is circular
  DISULFID 5..11: note = Disulfide bond
MWKICSDWQNCWP

SEQ ID NO: 71 - protein, 13 aa, synthetic construct
  DISULFID 5..11: note = Disulfide bond
  REGION 5..11: note = Peptide is circular
MWHICQEWATCWP

SEQ ID NO: 72 - protein, 13 aa, synthetic construct
  REGION 5..11: note = Peptide is circular
  DISULFID 5..11: note = Disulfide bond
MWTLCDTWDTCFP

SEQ ID NO: 73 - protein, 13 aa, synthetic construct
  DISULFID 5..11: note = Disulfide bond
  REGION 5..11: note = Peptide is circular
MWTLCDNWQTCWP

SEQ ID NO: 74 - protein, 13 aa, synthetic construct
  REGION 5..10: note = Peptide is circular
  DISULFID 5..10: note = Disulfide bond
MWQLCEWNNCWPV

SEQ ID NO: 75 - protein, 13 aa, synthetic construct
  DISULFID 5..10: note = Disulfide bond
  REGION 5..10: note = Peptide is circular
MWVLCEWHECWPG

SEQ ID NO: 76 - protein, 13 aa, synthetic construct
  REGION 3..8: note = Peptide is circular
  DISULFID 3..8: note = Disulfide bond
MDCNFFDCDAWTW

SEQ ID NO: 77 - protein, 13 aa, synthetic construct
  DISULFID 5..11: note = Disulfide bond
  REGION 5..11: note = Peptide is circular
MWMLCDWDKDCWP

SEQ ID NO: 78 - protein, 13 aa, synthetic construct
  REGION 6..11: note = Peptide is circular
  DISULFID 6..11: note = Disulfide bond
MVWVPCEWDDCFY

SEQ ID NO: 79 - protein, 13 aa, synthetic construct
  DISULFID 4..10: note = Disulfide bond
  REGION 4..10: note = Peptide is circular
MSDCWLQWNCPFD

SEQ ID NO: 80 - protein, 12 aa, synthetic construct
  REGION 4..10: note = Peptide is circular
  DISULFID 4..10: note = Disulfide bond
WTICNNWHDCWP

SEQ ID NO: 81 - protein, 13 aa, synthetic construct
  DISULFID 5..11: note = Disulfide bond
  REGION 5..11: note = Peptide is circular
  SITE 1: note = D-alanine
AWTICNNWHDCWP

SEQ ID NO: 82 - protein, 13 aa, synthetic construct
  DISULFID 5..11: note = Disulfide bond
  REGION 5..11: note = Peptide is circular
  SITE 6: note = D-alanine
MWTICANWHDCWP

SEQ ID NO: 83 - protein, 13 aa, synthetic construct
  SITE 10: note = D-alanine
  REGION 5..11: note = Peptide is circular
  DISULFID 5..11: note = Disulfide bond
MWTICNNWHACWP

SEQ ID NO: 84 - protein, 13 aa, synthetic construct
  DISULFID 5..11: note = Disulfide bond
  REGION 5..11: note = Peptide is circular
  SITE 12: note = D-alanine
MWTICNNWHDCAP

SEQ ID NO: 85 - protein, 13 aa, synthetic construct
  SITE 13: note = D-alanine
  DISULFID 5..11: note = Disulfide bond
  REGION 5..11: note = Peptide is circular
MWTICNNWHDCWA

SEQ ID NO: 86 - protein, 13 aa, synthetic construct
  REGION 5..11: note = Peptide is circular
  DISULFID 5..11: note = Disulfide bond
AWTICNNWHDCWP

SEQ ID NO: 87 - protein, 13 aa, synthetic construct
  DISULFID 5..11: note = Disulfide bond
  REGION 5..11: note = Peptide is circular
MWAICNNWHDCWP

SEQ ID NO: 88 - protein, 13 aa, synthetic construct
  REGION 5..11: note = Peptide is circular
  DISULFID 5..11: note = Disulfide bond
MWTACNNWHDCWP

SEQ ID NO: 89 - protein, 13 aa, synthetic construct
  DISULFID 5..11: note = Disulfide bond
  REGION 5..11: note = Peptide is circular
MWTICANWHDCWP

SEQ ID NO: 90 - skipped

SEQ ID NO: 91 - protein, 13 aa, synthetic construct
  REGION 4..10: note = Peptide is circular
  DISULFID 4..10: note = Disulfide Bond
  MOD_RES 13: note = D-lysine-(1,4,7,10-tetra-azacylcododecane-1,4,7,10- tetraacetic acid
  SITE 13: note = D-Lysine
WTICNNWHDCWPX

SEQ ID NO: 92 - protein, 14 aa, synthetic construct
  REGION 4..10: note = Peptide is circular
  DISULFID 4..10: note = Disulfide bond
  SITE 13: note = D-Lysine
  MOD_RES 14: note = 6-aminohexanoic acid
WTICNNWHDCWPKX

SEQ ID NO: 93 - protein, 14 aa, synthetic construct
  REGION 4..10: note = Peptide is circular
  REGION 4..10: note = Disulfide bond
  SITE 13: note = D-Lysine
  MOD_RES 14: note = (4-aminocyclohexyl)acetic acid
WTICNNWHDCWPKX

SEQ ID NO: 94 - protein, 13 aa, synthetic construct
  REGION 4..10: note = Peptide is circular
  REGION 4..10: note = Disulfide bond
  MOD_RES 7: note = 1-napthyl-alanine
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 95 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 96 - protein, 13 aa, synthetic construct
  REGION 4..10: note = Peptide is circular
  DISULFID 4..10: note = Disulfide bond
  MOD_RES 11: note = 1-napthyl-alanine
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 97 - protein, 13 aa, synthetic construct
  REGION 4..10: note = Peptide is circular
  DISULFID 4..10: note = Disulfide bond
  MOD_RES 11: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 98 - protein, 13 aa, synthetic construct
  REGION 4..10: note = Peptide is circular
  DISULFID 4..10: note = Disulfide bond
  MOD_RES 7: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 99 - protein, 13 aa, synthetic construct
  REGION 4..10: note = Peptide is circular
  DISULFID 4..10: note = Disulfide bond
  MOD_RES 7: note = 5-methoxy-tryptophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 100 - protein, 13 aa, synthetic construct
  REGION 4..10: note = Peptide is circular
  DISULFID 4..10: note = Disulfide bond
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 101 - protein, 13 aa, synthetic construct
  REGION 4..10: note = Peptide is circular
  DISULFID 4..10: note = Disulfide bond
  MOD_RES 11: note = 5-methoxy-tryptophan
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 102 - protein, 14 aa, synthetic construct
  REGION 4..10: note = Peptide is circular
  DISULFID 4..10: note = Disulfide bond
  SITE 13: note = D-Lysine
  MOD_RES 14: note = 27-amino-4,7,10,13,16,19,22,25-octaoxaheptacosanoic acid
WTICNNWHDCWPKX

SEQ ID NO: 103 - protein, 13 aa, synthetic construct
  REGION 4..10: note = Peptide is circular
  DISULFID 4..10: note = Disulfide bond
  MOD_RES 5: note = (S)-2-amino-4-hexanamidobutanoic acid
  MOD_RES 13: note = D-lysine-(1,4,7,10-tetra-azacylcododecane-1,4,7,10- tetraacetic acid)
  SITE 13: note = D-Lysine
WTICXNWHDCWPX

SEQ ID NO: 104 - protein, 13 aa, synthetic construct
  REGION 4..10: note = Peptide is circular
  DISULFID 4..10: note = Disulfide bond
  MOD_RES 5: note = 2-amino-4-octanamidobutanoic acid
  MOD_RES 13: note = D-lysine-(1,4,7,10-tetra-azacylcododecane-1,4,7,10- tetraacetic acid)
  SITE 13: note = D-Lysine
WTICXNWHDCWPX

SEQ ID NO: 105 - protein, 13 aa, synthetic construct
  REGION 4..10: note = Peptide is circular
  DISULFID 4..10: note = Disulfide bond
  MOD_RES 8: note = (S)-2-amino-4-hexanamidobutanoic acid
  MOD_RES 13: note = D-lysine-(1,4,7,10-tetra-azacylcododecane-1,4,7,10- tetraacetic acid)
  SITE 13: note = D-Lysine
WTICNNWXDCWPX

SEQ ID NO: 106 - protein, 13 aa, synthetic construct
  REGION 4..10: note = Peptide is circular
  DISULFID 4..10: note = Disulfide bond
  MOD_RES 8: note = 2-amino-4-octanamidobutanoic acid
  MOD_RES 13: note = D-lysine-(1,4,7,10-tetra-azacylcododecane-1,4,7,10- tetraacetic acid)
  SITE 13: note = D-Lysine
WTICNNWXDCWPX

SEQ ID NO: 107 - protein, 13 aa, synthetic construct
  MOD_RES 1: note = 5-fluoro-tryptophan
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
XTICNNWHDCWPX

SEQ ID NO: 108 - protein, 13 aa, synthetic construct
  REGION 4..10: note = Peptide is circular
  DISULFID 4..10: note = Disulfide bond
  MOD_RES 7: note = 5-fluoro-tryptophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 109 - protein, 13 aa, synthetic construct
  REGION 4..10: note = Peptide is circular
  DISULFID 4..10: note = Disulfide bond
  MOD_RES 12: note = Alpha-Methyl-proline
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 110 - protein, 13 aa, synthetic construct
  MOD_RES 3: note = Cyclohexylglycine
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTXCNNWHDCWPX

SEQ ID NO: 111 - protein, 13 aa, synthetic construct
  MOD_RES 3: note = 1-napthyl-alanine
  MOD_RES 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTXCNNWHDCWPX

SEQ ID NO: 112 - protein, 13 aa, synthetic construct
  REGION 4..10: note = Peptide is circular
  DISULFID 4..10: note = Disulfide bond
  MOD_RES 12: note = Pipecolic acid
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 113 - protein, 13 aa, synthetic construct
  REGION 4..10: note = Peptide is circular
  DISULFID 4..10: note = Disulfide bond
  MOD_RES 12: note = 5,5-dimethyl-proline
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 114 - protein, 13 aa, synthetic construct
  REGION 4..10: note = Peptide is circular
  DISULFID 4..10: note = Disulfide bond
  MOD_RES 12: note = Cis-4-hydroxy-proline
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 115 - protein, 13 aa, synthetic construct
  REGION 4..10: note = Peptide is circular
  DISULFID 4..10: note = Disulfide bond
  MOD_RES 12: note = Trans-4-hydroxy-proline
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 116 - protein, 13 aa, synthetic construct
  REGION 4..10: note = Peptide is circular
  DISULFID 4..10: note = Disulfide bond
  MOD_RES 12: note = Cis-4-fluoro-proline
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 117 - protein, 13 aa, synthetic construct
  REGION 4..10: note = Peptide is circular
  DISULFID 4..10: note = Disulfide bond
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 118 - protein, 13 aa, synthetic construct
  REGION 4..10: note = Peptide is circular
  DISULFID 4..10: note = Disulfide bond
  MOD_RES 3: note = 2-(trifluromethyl)-phenylalanine
  MOD_RES 13: note = D-lysine-(1,4,7,10-tetra-azacylcododecane-1,4,7,10- tetraacetic acid)
  SITE 13: note = D-Lysine
WTXCNNWHDCWPX

SEQ ID NO: 119 - protein, 13 aa, synthetic construct
  MOD_RES 3: note = 2-phenylethyl-alanine
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTXCNNWHDCWPX

SEQ ID NO: 120 - protein, 13 aa, synthetic construct
  MOD_RES 3: note = 2-naphthyl-alanine
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTXCNNWHDCWPX

SEQ ID NO: 121 - protein, 13 aa, synthetic construct
  REGION 4..10: note = Peptide is circular
  DISULFID 4..10: note = Disulfide bond
  MOD_RES 11: note = Alpha-Methyl-trypotophan
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 122 - protein, 13 aa, synthetic construct
  REGION 4..10: note = Peptide is circular
  DISULFID 4..10: note = Disulfide bond
  MOD_RES 7: note = Alpha-Methyl-trypotophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 123 - protein, 14 aa, synthetic construct
  MOD_RES 1: note = (palmitic acid)-(glutamic acid)-(3-[2-[2-[2-[2-[2-[ 2-[2-[2-[2-[2-[2-(2-aminoethoxy)-ethoxy]ethoxy]ethoxy]etho xy]ethoxy]ethoxy]ethoxy]ethoxy]ethoxy]ethoxy]ethoxy]propan oic acid)
  REGION 5..11: note = Peptide is circular
  DISULFID 5..11: note = Disulfide bond
  MOD_RES 14: note = D-lysine-(1,4,7,10-tetra-azacylcododecane-1,4,7,10- tetraacetic acid)
  SITE 14: note = D-Lysine
XWTICNNWHDCWPX

SEQ ID NO: 124 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 125 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 126 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 127 - protein, 13 aa, synthetic construct
  MOD_RES 1: note = 5-fluoro-tryptophan
  SITE 3: note = Nle
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 128 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 129 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 130 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 131 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 132 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 133 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 134 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 135 - protein, 13 aa, synthetic construct
  MOD_RES 1: note = 5-fluoro-tryptophan
  SITE 3: note = Nle
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 136 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 137 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 138 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 139 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 4-Fluorophenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 140 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 141 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 142 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 143 - protein, 13 aa, synthetic construct
  MOD_RES 1: note = 5-fluoro-tryptophan
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
  SITE 3: note = Nle
XTXCNNXHDCXXX

SEQ ID NO: 144 - protein, 13 aa, synthetic construct
  MOD_RES 1: note = 5-fluoro-tryptophan
  MOD_RES 3: note = 2-amino-3-cyclohexylpropanoic acidd
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 145 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 146 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 147 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 148 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 149 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 150 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 151 - protein, 13 aa, synthetic construct
  MOD_RES 1: note = 5-fluoro-tryptophan
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
  SITE 3: note = Nle
XTXCNNXHDCXXX

SEQ ID NO: 152 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 153 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 154 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 155 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 4-(Trifluoromethyl)--phenylalanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 156 - protein, 13 aa, synthetic construct
  MOD_RES 1: note = 7-Methyl-tryptophan
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
XTICNNWHDCWPX

SEQ ID NO: 157 - protein, 13 aa, synthetic construct
  REGION 4..10: note = Peptide is circular
  DISULFID 4..10: note = Disulfide bond
  MOD_RES 12: note = N-methyl-Serine
  MOD_RES 13: note = D-lysine-(1,4,7,10-tetra-azacylcododecane-1,4,7,10- tetraacetic acid)
  SITE 13: note = D-Lysine
WTICNNWHDCWXX

SEQ ID NO: 158 - protein, 13 aa, synthetic construct
  REGION 4..10: note = Peptide is circular
  DISULFID 4..10: note = Disulfide bond
  MOD_RES 8: note = Lysine(tridecanoic acid)
  MOD_RES 13: note = D-lysine-(1,4,7,10-tetra-azacylcododecane-1,4,7,10- tetraacetic acid)
  SITE 13: note = D-Lysine
WTICNNWXDCWPX

SEQ ID NO: 159 - protein, 13 aa, synthetic construct
  REGION 4..10: note = Peptide is circular
  DISULFID 4..10: note = Disulfide bond
  MOD_RES 8: note = Lysine(pentadecanoic acid)
  MOD_RES 13: note = D-lysine-(1,4,7,10-tetra-azacylcododecane-1,4,7,10- tetraacetic acid)
  SITE 13: note = D-Lysine
WTICNNWXDCWPX

SEQ ID NO: 160 - protein, 13 aa, synthetic construct
  REGION 4..10: note = Peptide is circular
  DISULFID 4..10: note = Disulfide bond
  MOD_RES 8: note = Lysine(C16)
  MOD_RES 13: note = D-lysine-(1,4,7,10-tetra-azacylcododecane-1,4,7,10- tetraacetic acid)
  SITE 13: note = D-Lysine
WTICNNWXDCWPX

SEQ ID NO: 161 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNNWHDCWPX

SEQ ID NO: 162 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNNWHDCWPX

SEQ ID NO: 163 - protein, 13 aa, synthetic construct
  REGION 4..10: note = Peptide is circular
  CROSSLNK 4..10: note = Thioacetal bridge or methylene cross-linker
  MOD_RES 13: note = D-lysine-(1,4,7,10-tetra-azacylcododecane-1,4,7,10- tetraacetic acid)
  SITE 13: note = D-Lysine
WTICNNWHDCWPX

SEQ ID NO: 164 - protein, 13 aa, synthetic construct
  REGION 4..10: note = Peptide is circular
  CROSSLNK 4..10: note = Thioacetal bridge or methylene cross-linker
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 165 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = Lysine-(1,4,7,10-tetra-azacylcododecane-N,N,N,N-tet raacetic acid)
WTICNNXHDCWPX

SEQ ID NO: 166 - protein, 19 aa, synthetic construct
  REGION 4..10: note = Peptide is circular
  DISULFID 4..10: note = Disulfide bond
  MOD_RES 7: note = 2-naphthyl-alanine
  MOD_RES 19: note = D-lysine-(1,4,7,10-tetra-azacylcododecane-1,4,7,10- tetraacetic acid)
  SITE 19: note = D-Lysine
WTICNNXHDCWPGGTTDSX

SEQ ID NO: 167 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 168 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 169 - protein, 13 aa, synthetic construct
  MOD_RES 1: note = 5-fluoro-tryptophan
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
  SITE 3: note = Nle
XTXCNNXHDCXXX

SEQ ID NO: 170 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 171 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 172 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 173 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 174 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 175 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 176 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 177 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 178 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 179 - protein, 13 aa, synthetic construct
  MOD_RES 1: note = 5-fluoro-tryptophan
  SITE 3: note = Nle
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 180 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 181 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 1-napthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 182 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 183 - protein, 13 aa, synthetic construct
  MOD_RES 1: note = 5-fluoro-tryptophan
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
  SITE 3: note = Nle
XTXCNNXHDCXXX

SEQ ID NO: 184 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 185 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 186 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 187 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 188 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 189 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Azetidine-2-carboxylic acid
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 190 - protein, 13 aa, synthetic construct
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCNNXHDCXPX

SEQ ID NO: 191 - protein, 13 aa, synthetic construct
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCANXHDCXPX

SEQ ID NO: 192 - protein, 13 aa, synthetic construct
  MOD_RES 3: note = 2-amino-3-cyclohexylpropanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCANXHDCXPX

SEQ ID NO: 193 - protein, 13 aa, synthetic construct
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCANXHDCXPX

SEQ ID NO: 194 - protein, 13 aa, synthetic construct
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCNAXHDCXPX

SEQ ID NO: 195 - protein, 13 aa, synthetic construct
  MOD_RES 3: note = 2-amino-3-cyclohexylpropanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCNAXHDCXPX

SEQ ID NO: 196 - protein, 13 aa, synthetic construct
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-lysine-(1,4,7,10-tetra-azacylcododecane-1,4,7,10- tetraacetic acid)
  SITE 13: note = D-Lysine
WTXCNAXHDCXPX

SEQ ID NO: 197 - protein, 14 aa, synthetic construct
  REGION 5..11: note = Peptide is circular
  DISULFID 5..11: note = Disulfide bond
  MOD_RES 14: note = D-lysine-(1,4,7,10-tetra-azacylcododecane-1,4,7,10- tetraacetic acid)
  SITE 14: note = D-Lysine
MWTLCDWNSDCWPX

SEQ ID NO: 198 - protein, 13 aa, synthetic construct
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = 2,2',2''-(10-(2-(((1R,4r)-4-(2-(((R)-5-amino-5-carb oxypentyl)amino)-2-oxoethyl)cyclohexyl)amino)-2-oxoethyl)- 1,4,7,10-tetraazacyclododecane-1,4,7-triyl)triacetic acid
  SITE 13: note = D-Lysine
WTXCNNXHDCXPX

SEQ ID NO: 199 - protein, 13 aa, synthetic construct
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = 2,2',2''-(10-(2-(((1R,4r)-4-(2-(((R)-5-amino-5-carb oxypentyl)amino)-2-oxoethyl)cyclohexyl)amino)-2-oxoethyl)- 1,4,7,10-tetraazacyclododecane-1,4,7-triyl)triacetic acid
  SITE 13: note = D-Lysine
WTXCNNXHDCXPX

SEQ ID NO: 200 - protein, 13 aa, synthetic construct
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 13: note = D-Lysine-(Glycine-Glycine-(1-amino-15-oxo-4,7,10-tr ioxa-14-azaoctadecan-18-oyl))
  SITE 13: note = D-Lysine
WTXCNNXHDCXPX

SEQ ID NO: 201 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WKICNNXHDCWPX

SEQ ID NO: 202 - protein, 13 aa, synthetic construct
  MOD_RES 1: note = 7-Methyl-tryptophan
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
XTICNNXHDCWPX

SEQ ID NO: 203 - protein, 13 aa, synthetic construct
  MOD_RES 1: note = 7-Methyl-tryptophan
  REGION 4..10: note = Peptide is circular
  DISULFID 4..10: note = Disulfide bond
  MOD_RES 7: note = 2-naphthyl-alanine
  MOD_RES 13: note = (D-Lysine)-(glutamic acid)-(glutamic acid)-(glutamic acid)-(1,4,7,10-tetra-azacylcododecane-N,N,N,N-tetraacetic acid)
  SITE 13: note = D-Lysine
XTICNNXHDCWPX

SEQ ID NO: 204 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICSNXHDCWPX

SEQ ID NO: 205 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICNDXHDCWPX

SEQ ID NO: 206 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICNNXQDCWPX

SEQ ID NO: 207 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICNNXHNCWPX

SEQ ID NO: 208 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICSDXQNCWPX

SEQ ID NO: 209 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICNKXHDCWPX

SEQ ID NO: 210 - protein, 13 aa, synthetic construct
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
  MOD_RES 1: note = 7-Cl-W
XTICNNWHDCWPX

SEQ ID NO: 211 - protein, 13 aa, synthetic construct
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
  MOD_RES 12: note = MeGly
WTICNNWHDCWXX

SEQ ID NO: 212 - protein, 13 aa, synthetic construct
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
  MOD_RES 12: note = Azetidine-2-carboxylic acid
WTICNNWHDCWXX

SEQ ID NO: 213 - protein, 13 aa, synthetic construct
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
  MOD_RES 12: note = N-methyl-Alanine
WTICNNWHDCWXX

SEQ ID NO: 214 - protein, 13 aa, synthetic construct
  MOD_RES 7: note = 2-naphthyl-alanine
  MOD_RES 13: note = D-Lysine-(Glycine-Glycine-(1-amino-15-oxo-4,7,10-tr ioxa-14-azaoctadecan-18-oyl))
  SITE 13: note = D-Lysine
  REGION 4..10: note = Peptide is circular
  DISULFID 4..10: note = Disulfide bond
WTICNNXHDCWPX

SEQ ID NO: 215 - protein, 19 aa, synthetic construct
  MOD_RES 7: note = 2-naphthyl-alanine
  MOD_RES 19: note = D-lysine-(1,4,7,10-tetra-azacylcododecane-1,4,7,10- tetraacetic acid)
  SITE 19: note = D-Lysine
  REGION 4..10: note = Peptide is circular
  DISULFID 4..10: note = Disulfide bond
WTICNNXHDCWPGGSGGSX

SEQ ID NO: 216 - protein, 19 aa, synthetic construct
  MOD_RES 7: note = 2-naphthyl-alanine
  MOD_RES 19: note = D-lysine-(1,4,7,10-tetra-azacylcododecane-1,4,7,10- tetraacetic acid)
  SITE 19: note = D-Lysine
  REGION 4..10: note = Peptide is circular
  DISULFID 4..10: note = Disulfide bond
WTICNNXHDCWPGSGSGSX

SEQ ID NO: 217 - protein, 14 aa, synthetic construct
  MOD_RES 7: note = 2-naphthyl-alanine
  MOD_RES 14: note = D-lysine-(1,4,7,10-tetra-azacylcododecane-1,4,7,10- tetraacetic acid)
  SITE 14: note = D-Lysine
  REGION 4..10: note = Peptide is circular
  DISULFID 4..10: note = Disulfide bond
WTICNNXHDCWPEX

SEQ ID NO: 218 - protein, 16 aa, synthetic construct
  MOD_RES 7: note = 2-naphthyl-alanine
  MOD_RES 16: note = D-lysine-(1,4,7,10-tetra-azacylcododecane-1,4,7,10- tetraacetic acid)
  SITE 16: note = D-Lysine
  REGION 4..10: note = Peptide is circular
  DISULFID 4..10: note = Disulfide bond
WTICNNXHDCWPEECX

SEQ ID NO: 219 - protein, 15 aa, synthetic construct
  MOD_RES 7: note = 2-naphthyl-alanine
  MOD_RES 15: note = D-lysine-(1,4,7,10-tetra-azacylcododecane-1,4,7,10- tetraacetic acid)
  SITE 15: note = D-Lysine
  REGION 4..10: note = Peptide is circular
  DISULFID 4..10: note = Disulfide bond
  SITE 13: note = D-Proline
  MOD_RES 14: note = 27-amino-4,7,10,13,16,19,22,25-octaoxaheptacosanoic acid
WTICNNXHDCWPPXX

SEQ ID NO: 220 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
WTICNNXKDCWPX

SEQ ID NO: 221 - protein, 13 aa, synthetic construct
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
  MOD_RES 8: note = K(Me)3
WTICNNXXDCWPX

SEQ ID NO: 222 - protein, 13 aa, synthetic construct
  MOD_RES 7: note = 1-napthyl-alanine
  MOD_RES 13: note = D-lysine-(1,4,7,10-tetra-azacylcododecane-1,4,7,10- tetraacetic acid)
  SITE 13: note = D-Lysine
  REGION 4..10: note = Peptide is circular
  DISULFID 4..10: note = Disulfide bond
  MOD_RES 1: note = 5-fluoro-tryptophan
  MOD_RES 3: note = (S)-2-amino-3-ethylpentanoic acid
  MOD_RES 11: note = 7-aza-tryptophan
  MOD_RES 12: note = Azetidine-2-carboxylic acid
XTXCNNXHDCXXX

SEQ ID NO: 223 - protein, 13 aa, synthetic construct
  MOD_RES 1: note = 5-fluoro-tryptophan
  MOD_RES 3: note = (S)-2-amino-3-ethylpentanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 224 - protein, 13 aa, synthetic construct
  MOD_RES 1: note = 5-fluoro-tryptophan
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 225 - protein, 13 aa, synthetic construct
  MOD_RES 1: note = 5-fluoro-tryptophan
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
  SITE 3: note = Nle
XTXCNNXHDCXXX

SEQ ID NO: 226 - protein, 13 aa, synthetic construct
  MOD_RES 1: note = 5-fluoro-tryptophan
  MOD_RES 3: note = 2-amino-3-cyclohexylpropanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 227 - protein, 13 aa, synthetic construct
  MOD_RES 1: note = 5-fluoro-tryptophan
  MOD_RES 3: note = 2-(trifluromethyl)-phenylalanine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 228 - protein, 13 aa, synthetic construct
  MOD_RES 3: note = 2-amino-2-(tetrahydro-2H-pyran-4-yl)acetic acid
  REGION 4..10: note = Peptide is circular
  DISULFID 4..10: note = Disulfide bond
  MOD_RES 7: note = 2-naphthyl-alanine
  MOD_RES 13: note = (D-Lys)-(1-amino-3,6,9,12,15,18,21,24-octaoxaheptac osan-27-oic acid)-(D-Lysine)-(1,4,7,10-tetra-azacylcododec ane-N,N,N,N-tetraacetic acid)-(1-amino-3,6,9,12,15,18,21,2 4-octaoxaheptacosan-27-oic acid)-(2-(4-bromophenyl)acetamide)
  SITE 13: note = D-Lysine
WTICNNXHDCWPX

SEQ ID NO: 229 - protein, 13 aa, synthetic construct
  SITE 3: note = Nle
  REGION 4..10: note = Peptide is circular
  DISULFID 4..10: note = Disulfide bond
  MOD_RES 7: note = 2-naphthyl-alanine
  MOD_RES 13: note = (D-Lys)-(1-amino-3,6,9,12,15,18,21,24-octaoxaheptac osan-27-oic acid)-(D-Lysine)-(1,4,7,10-tetra-azacylcododec ane-N,N,N,N-tetraacetic acid)-(1-amino-3,6,9,12,15,18,21,2 4-octaoxaheptacosan-27-oic acid)-(2-(4-bromophenyl)acetamide)
  SITE 13: note = D-Lysine
WTICNNXHDCWPX

SEQ ID NO: 230 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = (D-Lys)-(1-amino-3,6,9,12,15,18,21,24-octaoxaheptac osan-27-oic acid)-(D-Lysine)-(1,4,7,10-tetra-azacylcododec ane-N,N,N,N-tetraacetic acid)-(1-amino-3,6,9,12,15,18,21,2 4-octaoxaheptacosan-27-oic acid)-(2-(4-bromophenyl)acetamide)
  SITE 13: note = D-Lysine
WTICNNXHDCWPX

SEQ ID NO: 231 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = pDBX
WTIXNNXHDCWPX

SEQ ID NO: 232 - protein, 13 aa, synthetic construct
  MOD_RES 1: note = 5-fluoro-tryptophan
  MOD_RES 3: note = Tert-butyl-glycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 233 - protein, 13 aa, synthetic construct
  MOD_RES 1: note = 5-fluoro-tryptophan
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 234 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 3-(4-piperidinyl)-alanine
WTICXNXHDCWPX

SEQ ID NO: 235 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = [3-(1-morpholinyl)-Alanine
WTICXNXHDCWPX

SEQ ID NO: 236 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 237 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 2-amino-3-(pyridin-3-yl)propanoic acid
WTICXNXHDCWPX

SEQ ID NO: 238 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 2-amino-3-(pyridin-4-yl)propanoic acid
WTICXNXHDCWPX

SEQ ID NO: 239 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 6: note = 2-amino-3-(pyridin-3-yl)propanoic acid
WTICNXXHDCWPX

SEQ ID NO: 240 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 6: note = 2-amino-3-(pyridin-4-yl)propanoic acid
WTICNXXHDCWPX

SEQ ID NO: 241 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 6: note = 3-(4-piperidinyl)-alanine
WTICNXXHDCWPX

SEQ ID NO: 242 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 6: note = [3-(1-morpholinyl)-Alanine
WTICNXXHDCWPX

SEQ ID NO: 243 - protein, 13 aa, synthetic construct
  MOD_RES 1: note = 5-fluoro-tryptophan
  MOD_RES 3: note = (S)-2-amino-3-cyclobutylpropanoic acid
  REGION 4..10: note = Peptide is circular
  DISULFID 4..10: note = Disulfide bond
  MOD_RES 7: note = 2-naphthyl-alanine
  MOD_RES 11: note = 7-aza-tryptophan
  MOD_RES 12: note = Trans-4-fluoro-proline
  MOD_RES 13: note = D-lysine-(1,4,7,10-tetra-azacylcododecane-1,4,7,10- tetraacetic acid)
  SITE 13: note = D-Lysine
XTXCNNXHDCXXX

SEQ ID NO: 244 - protein, 14 aa, synthetic construct
  REGION 4..10: note = Peptide is circular
  DISULFID 4..10: note = Disulfide bond
  MOD_RES 7: note = 2-naphthyl-alanine
  MOD_RES 14: note = D-lysine-(1,4,7,10-tetra-azacylcododecane-1,4,7,10- tetraacetic acid)
  SITE 14: note = D-Lysine
  MOD_RES 13: note = 1-amino-3,6,9,12-tetraoxapentadecan-15-oic acid
WTICNNXHDCWPXX

SEQ ID NO: 245 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = (D-Lys)-(1-amino-3,6,9,12-tetraoxapentadecan-15-oic acid)-(1,4,7,10-tetra-azacylcododecane-N,N,N,N-tetraacetic acid)
  SITE 13: note = D-Lysine
WTICNNXHDCWPX

SEQ ID NO: 246 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 11: note = 7-Cl-W
WTICNNWHDCXPX

SEQ ID NO: 247 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICENWEDCWPX

SEQ ID NO: 248 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNNWEDCWPX

SEQ ID NO: 249 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICEEWEDCWPX

SEQ ID NO: 250 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNEWEDCWPX

SEQ ID NO: 251 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICENWHDCWPX

SEQ ID NO: 252 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICEEWHDCWPX

SEQ ID NO: 253 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNEWHDCWPX

SEQ ID NO: 254 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 3: note = Cyclohexylglycine
  MOD_RES 5: note = 3-(4-piperidinyl)-alanine
  MOD_RES 7: note = 2-naphthyl-alanine
WTXCXNXHDCWPX

SEQ ID NO: 255 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 3: note = Tert-butyl-glycine
  MOD_RES 5: note = 3-(4-piperidinyl)-alanine
WTXCXNXHDCWPX

SEQ ID NO: 256 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 3: note = Tert-butyl-alanine
  MOD_RES 5: note = 3-(4-piperidinyl)-alanine
WTXCXNXHDCWPX

SEQ ID NO: 257 - protein, 13 aa, synthetic construct
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 3-(4-piperidinyl)-alanine
WTXCXNXHNCWPX

SEQ ID NO: 258 - protein, 13 aa, synthetic construct
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 3-(4-piperidinyl)-alanine
WTXCXNXHACWPX

SEQ ID NO: 259 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 3: note = Cyclohexylglycine
  MOD_RES 5: note = 3-(4-piperidinyl)-alanine
  MOD_RES 7: note = 2-naphthyl-alanine
WTXCXNXHTCWPX

SEQ ID NO: 260 - protein, 13 aa, synthetic construct
  REGION 4..10: note = Peptide is circular
  DISULFID 4..10: note = Disulfide bond
  MOD_RES 13: note = 2,2',2''-(10-(2-(((1R,4r)-4-(2-(((R)-5-amino-5-carb oxypentyl)amino)-2-oxoethyl)cyclohexyl)amino)-2-oxoethyl)- 1,4,7,10-tetraazacyclododecane-1,4,7-triyl)triacetic acid
  SITE 13: note = D-Lysine
  MOD_RES 3: note = Cyclohexylglycine
  MOD_RES 5: note = 3-(4-piperidinyl)-alanine
  MOD_RES 7: note = 2-naphthyl-alanine
WTXCXNXHDCWPX

SEQ ID NO: 261 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 3: note = Cyclohexylglycine
  MOD_RES 5: note = 2-amino-3-(pyridin-3-yl)propanoic acid
WTXCXNXHDCWPX

SEQ ID NO: 262 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 2-amino-3-(pyridin-3-yl)propanoic acid
  MOD_RES 3: note = Tert-butyl-glycine
WTXCXNXHDCWPX

SEQ ID NO: 263 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 3: note = Tert-butyl-alanine
  MOD_RES 5: note = 2-amino-3-(pyridin-3-yl)propanoic acid
WTXCXNXHDCWPX

SEQ ID NO: 264 - protein, 13 aa, synthetic construct
  MOD_RES 3: note = Cyclohexylglycine
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 5: note = 2-amino-3-(pyridin-4-yl)propanoic acid
WTXCXNXHDCWPX

SEQ ID NO: 265 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 3: note = Tert-butyl-glycine
  MOD_RES 5: note = 2-amino-3-(pyridin-4-yl)propanoic acid
WTXCXNXHDCWPX

SEQ ID NO: 266 - protein, 13 aa, synthetic construct
  REGION 4..10: note = Peptide is circular
  DISULFID 4..10: note = Disulfide bond
  MOD_RES 7: note = 5-Hydroxytryptophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 267 - protein, 13 aa, synthetic construct
  REGION 4..10: note = Peptide is circular
  DISULFID 4..10: note = Disulfide bond
  MOD_RES 11: note = 5-Hydroxytryptophan
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 268 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 1: note = 1-methyl-tryptophan
XTICNNWHDCWPX

SEQ ID NO: 269 - protein, 13 aa, synthetic construct
  REGION 4..10: note = Peptide is circular
  DISULFID 4..10: note = Disulfide bond
  MOD_RES 7: note = 7-Methyl-tryptophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 270 - protein, 13 aa, synthetic construct
  REGION 4..10: note = Peptide is circular
  DISULFID 4..10: note = Disulfide bond
  MOD_RES 7: note = 7-Methyloxy-tryptophan
  MOD_RES 13: note = D-lysine-(1,4,7,10-tetra-azacylcododecane-1,4,7,10- tetraacetic acid)
  SITE 13: note = D-Lysine
WTICNNXHDCWPX

SEQ ID NO: 271 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 7: note = 7-Cl-W
WTICNNXHDCWPX

SEQ ID NO: 272 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 11: note = 7-Methyl-tryptophan
WTICNNWHDCXPX

SEQ ID NO: 273 - protein, 13 aa, synthetic construct
  REGION 4..10: note = Peptide is circular
  DISULFID 4..10: note = Disulfide bond
  MOD_RES 11: note = 7-Methyloxy-tryptophan
  MOD_RES 13: note = D-lysine-(1,4,7,10-tetra-azacylcododecane-1,4,7,10- tetraacetic acid)
  SITE 13: note = D-Lysine
WTICNNWHDCXPX

SEQ ID NO: 274 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 5: note = 3-(4-piperidinyl)-alanine
  MOD_RES 7: note = 2-naphthyl-alanine
  MOD_RES 8: note = Lysine(tridecanoic acid)
WTICXNXXDCWPX

SEQ ID NO: 275 - protein, 13 aa, synthetic construct
  REGION 4..10: note = Peptide is circular
  MOD_RES 7: note = 2-naphthyl-alanine
  MOD_RES 13: note = Lysine-(1,4,7,10-tetra-azacylcododecane-N,N,N,N-tet raacetic acid)
  CROSSLNK 4..10: note = Thioacetal bridge or methylene cross-linker
WTICNNXHDCWPX

SEQ ID NO: 276 - protein, 13 aa, synthetic construct
  REGION 4..10: note = Peptide is circular
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 1: note = 7-Methyl-tryptophan
  CROSSLNK 4..10: note = Thioacetal bridge or methylene cross-linker
XTICNNXHDCWPX

SEQ ID NO: 277 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 1: note = 7-Methyloxy-tryptophan
WTXCXNXHDCWPX

SEQ ID NO: 278 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 12: note = N-methyl-Leucine
WTICNNWHDCWXX

SEQ ID NO: 279 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 3: note = Lysine(tridecanoic acid)
WTXCNNWHDCWPX

SEQ ID NO: 280 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 3: note = Lysine(pentadecanoic acid)
WTXCNNWHDCWPX

SEQ ID NO: 281 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 3: note = K(C16)
WTXCNNWHDCWPX

SEQ ID NO: 282 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNNWHDCWPX

SEQ ID NO: 283 - protein, 12 aa, synthetic construct
  REGION 4..10: note = Peptide is circular
  DISULFID 4..10: note = Disulfide bond
  MOD_RES 7: note = 2-naphthyl-alanine
  MOD_RES 12: note = (N-methyl-lysine)-(1,4,7,10-tetra-azacylcododecane- N,N,N,N-tetraacetic acid)
WTICNNXHDCWX

SEQ ID NO: 284 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-Lysine-(Glycine-Serine-Glycine-Serine-Glycine-Ser ine-(1,4,7,10-tetra-azacylcododecane-N,N,N,N-tetraacetic acid))
  SITE 13: note = D-Lysine
WTICNNXHDCWPX

SEQ ID NO: 285 - protein, 13 aa, synthetic construct
  REGION 4..10: note = Peptide is circular
  DISULFID 4..10: note = Disulfide bond
  MOD_RES 13: note = (D-Lysine)-(glutamic acid)-(1,4,7,10-tetra-azacylcododecane-N,N,N,N-tetraacetic acid)
  SITE 13: note = D-Lysine
  MOD_RES 7: note = 2-naphthyl-alanine
WTICNNXHDCWPX

SEQ ID NO: 286 - protein, 13 aa, synthetic construct
  REGION 4..10: note = Peptide is circular
  DISULFID 4..10: note = Disulfide bond
  MOD_RES 13: note = (D-Lysine)-(glutamic acid)-(glutamic acid)-(glutamic acid)-(1,4,7,10-tetra-azacylcododecane-N,N,N,N-tetraacetic acid)
  SITE 13: note = D-Lysine
  MOD_RES 7: note = 2-naphthyl-alanine
WTICNNXHDCWPX

SEQ ID NO: 287 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = (D-Lysine)-(D-Proline)-(1-amino-3,6,9,12,15,18,21,2 4-octaoxaheptacosan-27-oic acid)-(1,4,7,10-tetra-azacylcododecane-N,N,N,N-tetraacetic acid)
  SITE 13: note = D-Lysine
WTICNNXHDCWPX

SEQ ID NO: 288 - protein, 17 aa, synthetic construct
  REGION 4..10: note = Peptide is circular
  DISULFID 4..10: note = Disulfide bond
  MOD_RES 7: note = 2-naphthyl-alanine
  MOD_RES 17: note = D-lysine-(1,4,7,10-tetra-azacylcododecane-1,4,7,10- tetraacetic acid)
  SITE 17: note = D-Lysine
  SITE 13..15: note = D-Proline
  MOD_RES 16: note = 27-amino-4,7,10,13,16,19,22,25-octaoxaheptacosanoic acid
WTICNNXHDCWPPPPXX

SEQ ID NO: 289 - protein, 13 aa, synthetic construct
  REGION 4..10: note = Peptide is circular
  DISULFID 4..10: note = Disulfide bond
  MOD_RES 7: note = 2-naphthyl-alanine
  SITE 13: note = (D-Lysine)-(D-Proline)-(1-amino-3,6,9,12,15,18,21,2 4-octaoxaheptacosan-27-oic acid)-(1,4,7,10-tetra-azacylcododecane-N,N,N,N-tetraacetic acid)
WTICNNXHDCWPX

SEQ ID NO: 290 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 12: note = (2S,3R)-3-methylazetidine-2-carboxylic acid
WTICNNXHDCWXX

SEQ ID NO: 291 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 12: note = 2-methylazetidine-2-carboxylic acid
  MOD_RES 7: note = 2-naphthyl-alanine
WTICNNXHDCWXX

SEQ ID NO: 292 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 7: note = 2-naphthyl-alanine
  MOD_RES 12: note = (1S,3aR,6aS)-octahydrocyclopenta[c]pyrrole-1-carbox ylic acid
WTICNNXHDCWXX

SEQ ID NO: 293 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 12: note = 3,3-dimethylazetidine-2-carboxylic acid
WTICNNXHDCWXX

SEQ ID NO: 294 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = (D-Lys)-(1-amino-3,6,9,12,15,18,21,24-octaoxaheptac osan-27-oic acid)-(D-Lysine)-(1,4,7,10-tetra-azacylcododec ane-N,N,N,N-tetraacetic acid)
  SITE 13: note = D-Lysine
WTICNNXHDCWPX

SEQ ID NO: 295 - protein, 15 aa, synthetic construct
  REGION 4..10: note = Peptide is circular
  DISULFID 4..10: note = Disulfide bond
  MOD_RES 7: note = 2-naphthyl-alanine
  MOD_RES 15: note = D-lysine-(1,4,7,10-tetra-azacylcododecane-1,4,7,10- tetraacetic acid)
  SITE 15: note = D-Lysine
  MOD_RES 13: note = 2-amino-N-(carboxymethyl)-N,N-dimethylethan-1-amini um
  MOD_RES 14: note = 27-amino-4,7,10,13,16,19,22,25-octaoxaheptacosanoic acid
WTICNNXHDCWPXXX

SEQ ID NO: 296 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-Lysine-(Glycine-Glycine-Serine-Glycine-Glycine-Se rine-(1,4,7,10-tetra-azacylcododecane-N,N,N,N-tetraacetic acid))
  SITE 13: note = D-Lysine
WTICNNXHDCWPX

SEQ ID NO: 297 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 7: note = 2-naphthyl-alanine
  MOD_RES 8: note = 2-amino-3-(pyridin-3-yl)propanoic acid
WTICNNXXDCWPX

SEQ ID NO: 298 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 7: note = 2-naphthyl-alanine
  MOD_RES 8: note = 2-amino-3-(pyridin-4-yl)propanoic acid
WTICNNXXDCWPX

SEQ ID NO: 299 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = (D-Lysine)-(glutamic acid)-(glutamic acid)-(glutamic acid)-(1,4,7,10-tetra-azacylcododecane-N,N,N,N-tetraacetic acid)
  SITE 13: note = D-Lysine
  MOD_RES 8: note = Lysine(tridecanoic acid)
WTICNNXXDCWPX

SEQ ID NO: 300 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 1: note = 7-Methyl-tryptophan
XTICNNXHDCWPX

SEQ ID NO: 301 - protein, 18 aa, synthetic construct
  REGION 4..10: note = Peptide is circular
  DISULFID 4..10: note = Disulfide bond
  MOD_RES 18: note = D-lysine-(1,4,7,10-tetra-azacylcododecane-1,4,7,10- tetraacetic acid)
  SITE 18: note = D-Lysine
  MOD_RES 7: note = 2-naphthyl-alanine
  MOD_RES 13..17: note = MeGly
WTICNNXHDCWPXXXXXX

SEQ ID NO: 302 - protein, 15 aa, synthetic construct
  REGION 4..10: note = Peptide is circular
  DISULFID 4..10: note = Disulfide bond
  MOD_RES 7: note = 2-naphthyl-alanine
  MOD_RES 15: note = D-lysine-(1,4,7,10-tetra-azacylcododecane-1,4,7,10- tetraacetic acid)
  SITE 15: note = D-Lysine
  MOD_RES 13..14: note = 6-aminohexanoic acid
WTICNNXHDCWPXXX

SEQ ID NO: 303 - protein, 14 aa, synthetic construct
  REGION 4..10: note = Peptide is circular
  DISULFID 4..10: note = Disulfide bond
  MOD_RES 7: note = 2-naphthyl-alanine
  MOD_RES 14: note = D-lysine-(1,4,7,10-tetra-azacylcododecane-1,4,7,10- tetraacetic acid)
  SITE 14: note = D-Lysine
  MOD_RES 13: note = (1R,4r)-4-(((1r,4R)-4-(aminomethyl)cyclohexane-1-ca rboxamido)methyl)cyclohexane-1-carboxylic acid
WTICNNXHDCWPXX

SEQ ID NO: 304 - protein, 14 aa, synthetic construct
  REGION 4..10: note = Peptide is circular
  DISULFID 4..10: note = Disulfide bond
  MOD_RES 14: note = D-lysine-(1,4,7,10-tetra-azacylcododecane-1,4,7,10- tetraacetic acid)
  SITE 14: note = D-Lysine
  MOD_RES 7: note = 2-naphthyl-alanine
  MOD_RES 13: note = 4-(aminomethyl)benzoic acid
WTICNNXHDCWPXX

SEQ ID NO: 305 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WTICNNXRDCWPX

SEQ ID NO: 306 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  SITE 8: note = Orn
WTICNNXXDCWPX

SEQ ID NO: 307 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 3: note = Alpha-Methyl-Isoleucine
WTXCNNWHDCWPX

SEQ ID NO: 308 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
  MOD_RES 9: note = Alpha-Methyl-Aspartic Acid
WTICNNWHXCWPX

SEQ ID NO: 309 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICSNWSDCWPX

SEQ ID NO: 310 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICSSWSDCWPX

SEQ ID NO: 311 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICNSWSDCWPX

SEQ ID NO: 312 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICSNWHDCWPX

SEQ ID NO: 313 - protein, 13 aa, synthetic construct
  REGION 4..10: note = Peptide is circular
  DISULFID 4..10: note = Disulfide bond
  MOD_RES 13: note = D-lysine-(1,4,7,10-tetra-azacylcododecane-1,4,7,10- tetraacetic acid)
  SITE 13: note = D-Lysine
WTICSSWHDCWPX

SEQ ID NO: 314 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  MOD_RES 3: note = Cyclohexylglycine
  MOD_RES 5: note = 3-(4-piperidinyl)-alanine
  MOD_RES 8: note = Lysine-(1-AMINO-3,6,9,12,15,18,21,24,27,30,33,36,39 ,42,45,48,51,54,57,60,63,66,69,72-TETRACOSAOXAPENTAHEPTACO NTAN-75-OIC ACID)-(glutamic acid)-(hexadecanedioic acid)
WTXCXNXXDCWPX

SEQ ID NO: 315 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
  SITE 3: note = N-methyl-norleucine
  MOD_RES 5: note = 3-(4-piperidinyl)-alanine
  MOD_RES 8: note = Lysine-(1-AMINO-3,6,9,12,15,18,21,24,27,30,33,36,39 ,42,45,48,51,54,57,60,63,66,69,72-TETRACOSAOXAPENTAHEPTACO NTAN-75-OIC ACID)-(glutamic acid)-(hexadecanedioic acid)
WTXCXNXXDCWPX

SEQ ID NO: 316 - protein, 13 aa, synthetic construct
  REGION 4..10: note = Peptide is circular
  DISULFID 4..10: note = Disulfide bond
  MOD_RES 7: note = 2-naphthyl-alanine
  MOD_RES 13: note = D-lysine-(1,4,7,10-tetra-azacylcododecane-1,4,7,10- tetraacetic acid)
  SITE 13: note = D-Lysine
WTICKNXHDCWPX